SEQUENCE LISTING (8 sequences)

SEQ ID NO: 1 - DNA, 20 bp, synthetic construct
  source 1..20: mol_type = other DNA
agagtttgatcatggctcag

SEQ ID NO: 2 - DNA, 17 bp, synthetic construct
  source 1..17: mol_type = other DNA
ccagcagccgcggtaat

SEQ ID NO: 3 - DNA, 17 bp, synthetic construct
  source 1..17: mol_type = other DNA
cctacgggaggcagcag

SEQ ID NO: 4 - DNA, 18 bp, synthetic construct
  source 1..18: mol_type = other DNA
actcaaatgaattgacgg

SEQ ID NO: 5 - DNA, 16 bp, synthetic construct
  source 1..16: mol_type = other DNA
ccagcagccgcggtaa

SEQ ID NO: 6 - DNA, 20 bp, synthetic construct
  source 1..20: mol_type = other DNA
attagataccctggtagtcc

SEQ ID NO: 7 - DNA, 17 bp, synthetic construct
  source 1..17: mol_type = other DNA
aacgcgaagaaccttac

SEQ ID NO: 8 - DNA, 22 bp, synthetic construct
  source 1..22: mol_type = other DNA
aagtcgtaacaaggtaaccgta

(19) United States
(12) Patent Application Publication
(10) Pub. No.: US 2025/0207207 A1
(43) Pub. Date: Jun. 26, 2025

(54) TAXONOMIC SIGNATURES AND METHODS OF DETERMINING THE SAME

(71) Applicant: Baylor College of Medicine, Houston, TX (US)

(72) Inventors: Tor Savidge, League City, TX (US); Qinglong Wu, Houston, TX (US)

(73) Assignee: Baylor College of Medicine, Houston, TX (US)

(21) Appl. No.: 18/851,355

(22) PCT Filed: Mar. 27, 2023
(86) PCT No.: PCT/US2023/064978

§ 371 Date: Sep. 26, 2024

Related U.S. Application Data
(60) Provisional application No. 63/324,345, filed on Mar. 28, 2022.

(51) Int. Cl. C12Q 1/689

(52) U.S. Cl. CPC C12Q 1/689; C12Q 2600/112; C12Q 2600/158

(57) ABSTRACT

Methods of data analysis, methods of detection, methods of treatment, compositions, and kits associated with treatment of diarrheal disorders are disclosed herein. Inventive concepts disclosed herein can assist stake holders in relieving patient suffering by identifying appropriate therapeutic regimens in an individualized and/or accurate manner.

### CROSS-REFERENCE TO RELATED APPLICATIONS

This application claims priority to U.S. Provisional Patent Application Ser. No. 63/324,345, filed Mar. 28, 2022, which is incorporated by reference herein in its entirety.

### STATEMENT REGARDING FEDERALLY SPONSORED RESEARCH OR DEVELOPMENT

This invention was made with government support under NIAID U01-AI24290 and P01-A1152999 awarded by National Institutes of Health. The government has certain rights in the invention.

### SEQUENCE LISTING

The instant application contains a Sequence Listing which has been submitted in ST26 format and is hereby incorporated by reference in its entirety. Said ST26 copy, created on Mar. 27, 2023, is named SeqLst_BAYM0359WO.xml and is 7.914 bytes in size.

### TECHNICAL FIELD AND BACKGROUND

This disclosure relates to the fields of bacteriology, cell biology, physiology, molecular biology, bioinformatics, diagnostics, and medicine.

Targeted metagenomic sequencing is routinely used to identify disease-causing bacteria, archaea and fungi. 16S rRNA gene surveys are also emerging as components of strategies to define disease-specific microbiome markers and are being adopted as diagnostic tools to profile microbiome communities that contribute to clinical pathogenesis¹. However, in order to understand what constitutes a disease-associated or disease-causing microbiome, there is a need to define healthy human microbiome community characteristics and functions across diverse genetic and environmental confounders². Current approaches often yields inconsistent or conflicting results due to inadequate study power and experimental bias. Disclosed herein are methods directed to solving the aforementioned problem, wherein adequate study power is achieved and experimental bias is minimized to improve the consistency of results.

To this end, consortium-driven studies including MetaHIT. Human Microbiome Project. LifeLines and American Gut have made significant inroads into compositional profiling of the human gut microbiome by establishing standard operating procedures that include DNA extraction protocols, 16S primer design and bioinformatics pipelines³. The next phase in identifying robust host-microbiome interactions that modulate human disease requires integrated and sufficiently powered multi-center trials to account for human genetic and environmental variation, but optimal study designs are often logistically impossible and cost prohibitive. Reanalyzing large deposits of publicly available 16S sequencing data represents an alternative approach to mine clinical microbiome associations in order to facilitate precision diagnosis and microbiome-based therapy. Unfortunately, re-analysis of individual microbiome surveys remains a significant bioinformatics challenge due to the lack of appropriate analytical pipelines that provide accurate taxonomic profiling of sequences generated from distinct 16S variable regions across multiple technology platforms. Disclosed herein are methods directed to solving the aforementioned problem, wherein accurate taxonomic profiling of sequences generated form distinct 16S variable regions across multiple technology platforms is achieved.

Gastrointestinal disease is a notable example where clinical microbiome surveys have provided promising insights into microbiome-associations and mechanisms, but systematic review of these largely single-site cohort studies have demonstrated inconsistent findings, in large part due to variations in methods for data generation and analysis as they introduce significant bias for cross-comparisons^{4,5}. Chronic diarrhea is a major cause of morbidity in the developed world and overlapping disease symptoms are often difficult to diagnose and manage. Thus, there is a need for non-invasive approaches to help differentiate the clinical spectra of common diarrheal symptoms, especially in irritable bowel syndrome (IBS), inflammatory bowel diseases (IBD) including Crohn's disease (CD) and ulcerative colitis (UC), and Clostridiodes difficile infection (CDI) which afflict up to 20% of the population and for which misdiagnosis is frequent. With few reliable disease-specific fecal biomarkers reported for IBD or IBS^{6,7}, endoscopy remains the gold standard diagnosis combined with laboratory testing and questionnaire. Clinical diagnosis and treatment is complicated further by antibiotic use and susceptibility to CDI which is often a post-infectious trigger of IBS, while IBD patients are often asymptomatically colonized by toxigenic C. difficile^{8,9}. Disclosed herein are methods directed to solving the aforementioned problem, wherein accurate diagnosis of IBS, IBD (e.g., CD and/or UC), and/or CDI is achieved, thus providing the field with suitable methods for treating the same.

Herein the inventors describe at least methods and compositions that utilize 16S sequencing data to solve the aforementioned problems to provide stake holders with accurate diagnostic and/or method of treatment options for patients experiencing microbiome dysbiosis and/or diarrhea. In some embodiments, the information garnered from 16S sequencing data analysis can be applied to other sequencing and/or detection methods to provide stake holders with accurate diagnostic and/or method of treatment options for patients experiencing microbiome dysbiosis and/or diarrhea.

### SUMMARY

Given the risk for antimicrobial resistant (AMR)-pathogens causing life-threating infections and/or quality of life diminishment associated with improper diagnosis, successful infectious disease management is critically dependent on identifying the most susceptible patient and determining the appropriate therapeutic regimen to facilitate rapid clinical intervention. Although the value of precision diagnosis and treatment is well-recognized, neither the current analytical technology nor our understanding of microbiome feature associations are sufficiently well developed to initiate effective implementation.

The present disclosure is directed to methods and compositions that provide for accurate diagnosis and treatment of underlying microbiome dysbiosis in an individual. The methods can determine if an individual has CDI, IBS, IBD UC, or IBD CD. The methods can determine if an individual is at risk for CDI, IBS, IBD UC, or IBD CD. Embodiments of the disclosure provide methods of identifying individuals that have CDI, IBS, IBD UC, or IBD CD (compared to age-matched or sex-matched individuals in the general population who are considered to have a non-dysbiosed microbiome) and identifying individuals that do not have CDI, IBS, IBD UC, or IBD CD (compared to the general population who are considered to have a non-dysbiosed microbiome).

Methods described herein can include treating an individual having diarrhea comprising: measuring for one or more taxonomical features from a biological sample from the individual; and reducing the administration of antibiotics and/or antimicrobial treatment to the individual when the individual has presence or absence or a certain level of one or more feature(s) indicative of non-CDI causative diarrhea, or administering antibiotics and/or antimicrobial treatment to the individual when the individual has presence or absence or a certain level of one or more feature(s) indicative of pathogenic infection.

Methods can comprise antibiotics and/or antimicrobial treatment comprising at least one of the antibiotics selected from a small molecule antibiotic, an antibiotic derived from a natural product, a microbial composition, an antibody suitable for neutralizing pathogenic infections, a therapeutic, contact isolation, and any combination thereof.

Methods can comprise the proviso that if the non-CDI causative diarrhea is irritable bowel syndrome (IBS), administration of the antibiotic and/or antimicrobial rifaximin is not reduced. Methods can comprise antibiotics and/or antimicrobial treatments comprising at least one of vancomycin, fidaxomicin, and bezlotoxumab. Methods can comprise treatment with fidaxomicin, and optionally the treatment dosage is at least 200 mg twice daily for 10 days, the treatment is vancomycin, and optionally the treatment dosage is at least 125 mg four times per day for 10 days, and/or the treatment is bezlotoxumab.

Methods can comprise pathogenic diarrhea classification or non-CDI causative diarrhea classification characterized by measuring the presence, absence, and/or relative quantity of at least, exactly, or at most 10, 20, 40, 60, 80, 100, or greater than 100, or any range derivable therein, taxonomical features described in any one or more of Tables 9-17. In certain methods, characterization using taxonomical features described in Tables 9-17 is sequential. Methods can comprise more than one characterization using Tables 9-17, comprising first characterizing using Tables 10 and/or 12, followed by characterization using one or more of the remaining Tables.

Methods can comprise measuring of one or more taxonomical features comprising at least one of analyzing one or more nucleic acids in the sample, analyzing one or more metabolites in the sample, and analyzing one or more proteins in the sample. Methods can comprise nucleic acid analysis, wherein the nucleic acid is analyzed by sequencing, polymerase chain reaction, isothermal amplification, bioinformatics, or any combination thereof. Methods can comprise 16S ribosomal RNA analysis. Methods can comprise metabolite analysis by mass spectrometry, ELISA, chromatography, or any combination thereof. Methods can comprise protein analysis by mass spectrometry, ELISA, chromatography, Western blotting, immunoprecipitation, immunoelectrophoresis, or any combination thereof.

Methods can comprise reducing the administration of antibiotics and/or antimicrobial treatments to the individual when the individual has presence or absence or a certain level of one or more feature(s) indicative of non-CDI causative diarrhea, the subject microbiome is further characterized to determine whether the non-CDI causative diarrhea is associated with irritable bowel syndrome (IBS) or inflammatory bowel disease (IBD), and treatment is modified accordingly. Methods can comprise identifying non-CDI causative diarrhea associated with IBD, wherein the IBD is further characterized to determine whether the IBD is Ulcerative Colitis (UC) or Crohn's Disease (CD), and treatment is modified accordingly.

Methods can comprise measuring of one or more taxonomical features from a biological sample from an individual comprising determining changes in relative abundance, compared to a reference healthy gut microbiome, of at least, at most, or exactly 1, 2, 3, 4, 5, 6, 7, 8, 9, 10, 11, 12, 13, 14, 15, 16, 17, 18, 19, 20, 21, 22, or all of: Sphingobacteriaceae Pedobacter; Saccharibacteria Incertae Sedis; Peptostreptococcaceae Intestinibacter; Bacillales Incertae Sedis Gemella; Veillonella infantium; Chloroplast Streptophyta; Caulobacter segnis; Methylophilaceae Methylophilus; Lactobacilluses Streptococcaceae; Burkholderiales Comamonadaceae; Burkholderia ambifaria; Caulobacteraceae Caulobacter; Betaproteobacteria; Phyllobacteriaceae Phyllobacterium; Burkholderiales Burkholderiaceae; Sphingomonadaceae; Sphingomonas; Prevotellamassilia timonensis; Collinsella aerofaciens; Adlercreutzia equolifaciens; Blautia hominis; and Dorea formicigenerans; wherein the change in taxonomical feature relative abundance is indicative of non-CDI causative diarrhea associated with IBS.

Methods can comprise measuring of one or more taxonomical features from a biological sample from an individual comprising determining changes in relative abundance, compared to a reference healthy gut microbiome, of at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, or all of: Acidaminococcaceae Acidaminococcus; Actinomycetales Actinomycetaceae; Bacillales Gemella; Bacilli Lactobacillus; Betaproteobacteria; Betaproteobacteria Burkholderiales; Bifidobacterium boum; Blautia hominis; Clostridiales Incertae Sedis XI Parvimonas; Enterobacteriaceae Cedecea; Erysipelotrichaceae Faecalicoccus; Faecalimonas umbilicata; Fusobacteriaceae Fusobacterium; Fusobacterium nucleatum; Gammaproteobacteria Enterobacterales; Lachnoclostridium [Clostridium] bolteae; Lachnospiraceae Lachnoclostridium; Megasphaera micronuciformis; Peptoniphilaceae Peptoniphilus; Peptostreptococcaceae Peptostreptococcus; Peptostreptococcaceae Romboutsia; Peptostreptococcaceae Terrisporobacter; Proteobacteria Gammaproteobacteria; Rosenbergiella collisarenosi; Rosenbergiella nectarea; Ruminococcaceae Intestinimonas; Ruminococcaceae Subdoligranulum; Solobacterium moorei; Veillonellaceae Veillonella; Alistipes shahii; Bacteroides cellulosilyticus; Bacteroides koreensis; Bacteroides thetaiotaomicron; Bacteroides xylanisolvens; Colidextribacter massiliensis; Coprococcus catus; Coprococcus eutactus; Enterobacterales Enterobacteriaceae; Faecalibacterium prausnitzii; Methanobrevibacter smithii; Phascolarctobacterium faecium; Romboutsia timonensis; and Turicibacter sanguinis; wherein the change in taxonomical feature relative abundance is indicative of non-CDI causative diarrhea associated with IBD.

Methods can comprise measuring of one or more taxonomical features from a biological sample from a pediatric individual comprising determining changes in relative abundance, compared to a reference healthy pediatric gut microbiome, of at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 56, 47, 48, 49, 50, 51, 52, 53, 54, 55, 56, 57, 58, 59, 60, 61, 62, 63, 64, 65, 66, or all of: Bacilli Lactobacillus; Peptostreptococcaceae Clostridioides; Enterococcaceae Enterococcus; Eggerthellaceae Eggerthella; Erysipelotrichaceae Erysipelatoclostridium; Lachnospiraceae Lachnoclostridium; Firmicutes Bacilli; Enterobacteriaceae Escherichia; Enterobacteriales Enterobacteriaceae; Streptococcaceae Streptococcus; Actinomycetaceae Schaalia; Clostridiaceae Hungatella; Clostridiaceae Clostridium; Corynebacteriaceae Corynebacterium; Veillonellaceae Veillonella; Actinomycetaceae Actinomyces; Fusobacteriaceae Fusobacterium; Erysipelotrichaceae Longicatena; Lachnospiraceae Clostridium XlVa; Lachnospiraceae Eisenbergiella; Peptostreptococcaceae Intestinibacter; Enterobacteriaceae Citrobacter; Peptoniphilaceae Finegoldia; Carnobacteriaceae Granulicatella; Coriobacteriaceae Eggerthella; Peptostreptococcaceae Peptostreptococcus; Drancourtella massiliensis; Peptoniphilaceae Anaerococcus; Bacillales Gemella; Lachnospiraceae Sellimonas; Peptoniphilaceae Peptoniphilus; Pasteurellaceae Rodentibacter; Clostridium sensu stricto; Lachnospiraceae Faecalimonas; Proteobacteria Gammaproteobacteria; Clostridiaceae Lactonifactor; Micrococcaceae Rothia; Leuconostocaceae Weissella; Peptostreptococcaceae Terrisporobacter; Peptostreptococcaceae Romboutsia; Peptostreptococcaceae Clostridium XI; Coriobacteriaceae Atopobium; Atopobiaceae Atopobium; Ruminococcaceae Anaeromassilibacillus; Porphyromonadaceae Parabacteroides; Rikenellaceae Alistipes; Lachnospiraceae Eubacterium rectale group; Lachnospiraceae Coprococcus; Lachnospiraceae Roseburia; Porphyromonadacede Odoribacter; Veillonellaceae Dialister; Ruminococcaceae Clostridium IV; Bacteroidia Bacteroidales; Coriobacteriaceae Collinsella; Coriobacteriales Coriobacteriaceae; Ruminococcaceae Clostridium leptum group; Enterobacteriaceae Shimwellia; Acidaminococcaceae Phascolarctobacterium; Staphylococcaceae Staphylococcus; Lachnospiraceae Anaerobutyricum; Erysipelotrichaceae Holdemania; Clostridiales Monoglobus; Porphyromonadaceae Barnesiella; Pasteurellales Pasteurellaceae; Clostridiales Clostridiaceae 1; and Bacteroidales Rikenellaceae; wherein the change in taxonomical feature relative abundance is indicative of CDI associated diarrhea.

Methods can comprise measuring of one or more taxonomical features from a biological sample from a individual comprising determining changes in relative abundance, compared to a reference healthy gut microbiome, of at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, or all of: Blautia stercoris; Saccharibacteria Incertae Sedis; Bacteroides plebeius; Bacteroides nordii; Eubacterium siraeum; Bacteroides cellulosilyticus; Burkholderiales Burkholderiaceae; Caulobacteraceae Caulobacter; Pelomonas aquatic; Burkholderiaceae Burkholderia; Caulobacter segnis; Burkholderia ambifaria; Eubacterium ventriosum; Firmicutes Clostridia; Oxalobacter formigenes; Burkholderiales Comamonadaceae; Veillonella infantium; Bacteroides thetaiotaomicron; Sphingobacteriaceae Pedobacter; Burkholderia thailandensis; Erysipelotrichaceae Turicibacter; Collinsella aerofaciens; Veillonellaceae Veillonella; Lachnospiraceae Lachnoclostridium; Blautia hominis; Gammaproteobacteria Enterobacterales; Bifidobacteriales Bifidobacteriaceae; Ruminococcaceae Intestinimonas; Faecalimonas umbilicata; Actinomycetales Actinomycetaceae; Actinobacteria Actinobacteria; Dorea formicigenerans; and Bacteria Proteobacteria; wherein the change in taxonomical feature relative abundance is indicative of non-CDI causative diarrhea associated with IBS.

Methods can comprise measuring of one or more taxonomical features from a biological sample from a individual comprising determining changes in relative abundance, compared to a reference healthy gut microbiome, of at least, at most, or exactly 1, 2, 3, 4, 5, 6, 7, 8, 9, 10, 11, 12, 13, 14, 15, 16, 17, 18, 19, 20, or all of: Lachnospiraceae Lachnoclostridium; Ruminococcaceae Intestinimonas; Acidaminococcaceae Acidaminococcus; Bacilli Lactobacillus; Actinomycetales Actinomycetaceae; Ruminococcaceae Subdoligranulum; Peptostreptococcaceae Romboutsia; Bifidobacterium boum; Bacillales Gemella; Peptostreptococcaceae Peptostreptococcus; Peptoniphilaceae Peptoniphilus; Clostridiales Incertae Sedis XI Parvimonas; Peptostreptococcaceae Terrisporobacter; Erysipelotrichaceae Faecalicoccus; Solobacterium moorei; Bacteroides xylanisolvens; Enterobacterales Enterobacteriaceae; Bacteroides koreensis; Bacteroides cellulosilyticus; Phascolarctobacterium faecium; and Bacteroides thetaiotaomicron, wherein the change in taxonomical feature relative abundance is indicative of non-CDI causative diarrhea associated with IBD UC.

Methods can comprise measuring of one or more taxonomical features from a biological sample from a individual comprising determining changes in relative abundance, compared to a reference healthy gut microbiome, of at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, or all of: Faecalimonas umbilicata; Lachnospiraceae Lachnoclostridium; Lachnoclostridium [Clostridium] bolteae; Proteobacteria Gammaproteobacteria; Bacilli Lactobacilluses; Actinomycetales Actinomycetaceae; Gammaproteobacteria Enterobacterales; Rosenbergiella collisarenosi; Rosenbergiella nectarea; Veillonellaceae Veillonella; Acidaminococcaceae Acidaminococcus; Blautia hominis; Bifidobacterium boum; Enterobacteriaceae Cedecea; Ruminococcaceae Intestinimonas; Peptostreptococcaceae Romboutsia; Fusobacteriaceae Fusobacterium; Fusobacterium nucleatum; Megasphaera micronuciformis; Romboutsia timonensis; Faecalibacterium prausnitzii; Coprococcus catus; Alistipes shahii; Bacteroides cellulosilyticus; Bacteroides thetaiotaomicron; Coprococcus eutactus; Turicibacter sanguinis; Colidextribacter massiliensis; and Methanobrevibacter smithii; wherein the change in taxonomical feature relative abundance is indicative of non-CDI causative diarrhea associated with IBD CD.

Methods can comprise measuring of one or more taxonomical features from a biological sample from a individual comprising determining changes in relative abundance, compared to a reference healthy gut microbiome, of at least, at most, or exactly 1, 2, 3, 4, 5, 6, 7, 8, 9, 10, 11, 12, 13, 14, 15, 16, or all of: Faecalimonas umbilicata; Blautia [Ruminococcus] gnavus; Lachnoclostridium [Clostridium] boltede; Erysipelatoclostridium ramosum; Veillonellaceae Veillonella; Enterobacterales Enterobacteriaceae; Blautia caecimuris; Fusobacteriaceae Fusobacterium; Fusobacterium nucleatum; Faecalibacterium prausnitzii; Ruminococcaceae Oscillibacter; Ruminococcaceae Clostridium IV; Romboutsia timonensis; Ruminococcaceae Pseudoflavonifractor; Erysipelotrichales Erysipelotrichaceae; and Methanobacteriaceae Methanobrevibacter; wherein the change in taxonomical feature relative abundance is indicative of non-CDI causative diarrhea associated with IBD CD.

Methods can comprise measuring of one or more taxonomical features from a biological sample from a individual comprising determining changes in relative abundance, compared to a reference healthy gut microbiome, of at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 56, 47, 48, 49, 50, 51, 52, 53, 54, 55, 56, 57, 58, 59, 60, 61, 62, or all of: Anaerostipes hadrus; Faecalibacterium prausnitzii; Lachnospiraceae Coprococcus; Lachnospiraceae [Eubacterium] rectale; Lachnospiraceae Roseburia; Lachnospiraceae Fusicatenibacter; Lachnospiraceae Dorea; Ruminococcaceae Ruminococcus; Roseburia inulinivorans; Dorea longicatena; Roseburia intestinalis; Lachnospiraceae Anaerostipes; Coprococcus comes; Lactobacillus rogosae; Romboutsia timonensis; Saccharibacteria Incertae Sedis; Eubacterium [Eubacterium] eligens; Bacteroides plebeius; Lachnospiraceae Blautia; Roseburia faecis; Blautia obeum; Coprococcus catus; Betaproteobacteria Burkholderiales; Prevotella copri; Burkholderiales Burkholderiaceae; Caulobacter segnis; Caulobacterales Caulobacteraceae; Burkholderiaceae Burkholderia; Burkholderia ambifaria; Prevotellaceae Prevotella; Burkholderiales Comamonadaceae; Burkholderia thailandensis; Alistipes obesi; Blautia stercoris; Ruminococcus bromii; Pelomonas aquatica; Peptostreptococcaceae Romboutsia; Bacteroides coprocola; Streptococcus thermophilus; Ruminococcaceae Oscillibacter; Alistipes ihumii; Ruminococcus callidus; Phyllobacteriaceae Phyllobacterium; Coprococcus eutactus; Peptostreptococcaceae Intestinibacter; Clostridioides difficile; Enterobacterales Enterobacteriaceae; Enterococcaceae Enterococcus; Peptostreptococcaceae Clostridium XI; Gammaproteobacteria Enterobacterales; Bacilli Lactobacillus; Eggerthella lenta; Erysipelatoclostridium [Clostridium] innocuum; Enterobacteriaceae Citrobacter; Lachnospiraceae Clostridium XIVa; Lachnoclostridium [Clostridium] bolteae; Erysipelatoclostridium ramosum; Hungatella effluvia; Veillonella parvula; Lactobacilluseae Lactobacillus; Blautia [Ruminococcus] gnavus; Enterococcus saccharolyticus; and Coriobacteriaceae Eggerthella, wherein the change in taxonomical feature relative abundance is indicative of non-CDI causative diarrhea associated with IBS.

Methods can comprise measuring of one or more taxonomical features from a biological sample from a individual comprising determining changes in relative abundance, compared to a reference healthy gut microbiome, of at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 56, 47, 48, 49, 50, or all of: Anaerostipes hadrus; Lachnospiraceae Dorea; Dorea longicatena; Lachnospiraceae Coprococcus; Lachnospiraceae Roseburia; Blautia obeum; Coprococcus comes; Roseburia intestinalis; Lactobacillus rogosae; Eubacterium [Eubacterium] eligens; Bacteroidia Bacteroidales; Peptostreptococcaceae Romboutsia; Coriobacteriaceae Collinsella; Acidaminococcaceae Acidaminococcus; Prevotellaceae Prevotella; Prevotella copri; Coriobacteriales Coriobacteriaceae; Ruminococcaceae Ruminococcus; Alistipes obesi; Betaproteobacteria Burkholderiales; Ruminococcus bromii; Lachnospiraceae Lachnoclostridium; Bifidobacterium adolescentis; Actinomycetales Actinomycetaceae; Ruminococcaceae Intestinimonas; Bacteroides eggerthii; Clostridioides difficile; Peptostreptococcaceae Clostridium XI; Enterobacteriaceae Citrobacter; Enterobacterales Enterobacteriaceae; Veillonella parvula; Enterococcaceae Enterococcus; Lactobacillies Enterococcaceae; Erysipelatoclostridium [Clostridium] innocuum; Pectobacterium carotovorum; Enterobacteriales Enterobacteriaceae; Bacteroides xylanisolvens; Enterococcus saccharolyticus; Clostridium paraputrificum; Salmonella enterica; Erysipelatoclostridium ramosum; Hungatella effluvia; Bacteroides koreensis; Bacilli Lactobacillus; Blautia product; Klebsiella quasipneumoniae; Ruthenibacterium lactatiformans; Veillonella dispar; Coriobacteriaceae Eggerthella; and Lachnospiraceae Hungatella; wherein the change in taxonomical feature relative abundance is indicative of non-CDI causative diarrhea associated with IBD.

Also described herein are complexes comprising a plurality of oligonucleotide primer sets hybridized to nucleic acid template sequences, wherein the nucleic acid template sequences are taxonomically specific sequences associated with taxonomical features identified in tables 9-17. Complexes can comprise at least 5 or at least 10 oligonucleotide primer sets are hybridized to nucleic acid template sequences.

Also described herein are kits for measuring for presence or absence or a certain level of one or more taxonomical feature(s) from a biological sample from an individual, comprising: (a) a plurality of sets of oligonucleotide primers, wherein each set of primers hybridize to a different nucleic acid template sequence for amplifying taxonomically specific sequences; and optionally (b) a polymerase enzyme; wherein the individual sets of oligonucleotide primers hybridize to a taxonomically specific sequence associated with the taxonomical features identified in tables 9-17.

A kit can comprise a master mix further comprises deoxynucleoside triphosphates; and at least one indicator for detecting an amplification product by a change in color or fluorescence. A kit can comprise deoxynucleoside triphosphates comprise dTTP, dGTP, dATP, dCTP and/or dUTP. A kit can comprise at least 5, at least 10, at least 20, at least 40, at least 60, at least 80, at least 100, at least 120, at least 140, at least 160, at least 180, or at least 200 individual sets of oligonucleotide primers. A kit can comprise individual sets of oligonucleotide primers bound to a support substrate. A kit can comprise oligonucleotide primers directed to at least 4, at least or exactly 10, at least or exactly 20, at least or exactly 30, at least or exactly 40, or more than 40, or any range derivable therein, taxonomically specific sequences associated with the following taxonomic features: Bacteroides; Eubacterium rectale; Ruminococcus; Faecalibacterium; Enterococcus; Enterobacteriaceae; Roseburia; Coprococcus; Dorea; Lachnoclostridium; Clostridium XIVa; Erysipelatoclostridium; Alistipes; Fusicatenibacter; Odoribacter; Lactobacillus; Anaerostipes; Collinsella; Clostridioides; Klebsiella; Agathobaculum butyriciproducens; Veillonella; Phascolarctobacterium; Adlercreutzia; Clostridium; Eggerthella; Sutterellaceae Parasutterella; barnesiella; Eubacterium; Clostridium IV; Gemmiger; Streptococcus; Dialister; Escherichia; Colidextribacter; Oxalobacter; Prevotella; Clostridium XVIII; Actinomyces; and Fusobacterium.

The individual may be of any kind, and the methods may be performed before, during, or after the individual has diarrhea. The methods may be performed when the individual is in need of antibiotics and/or antimicrobials of any kind or when the individual has already had antibiotics and/or antimicrobials of any kind. The methods may be performed as routine medical practice for an individual. The methods may be performed as preventative medical practice for an individual.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1D, describes how taxonomic and clustering accuracy are influenced by 16S amplicon sequence length, orientation and variable region. 1A) Depicts a schematic showing how taxonomic accuracy is improved by increasing amplicon length. 1B) Depicts Spearman correlations of VSEARCH-based de novo clustering with 99% similarity for 16S V1-V3 amplicons of variable length from the same parent 16S sequence. Orange boxes show the optimal sequence length range for clustering (FIGS. 7A-7D show results for other 16S variable regions; the associated Spearman correlation results for other clustering/denoising tools are available upon request). 1C) Depicts confidence score and 1D) depicts accuracy of taxonomic assignment for simulated amplicons; as shown, confidence and accuracy are significantly influenced by sequence length and orientation (FIGS. 9A-9D shows results for other 16S variable regions). “Org.” stands for original amplicon length without trimming; *=p<0.05 (Wilcoxon test; correct vs wrong genus/species annotations at each amplicon length).

FIGS. 2A-2D, describes the Taxa4Meta pipeline and associated taxonomic profiling of 16S amplicon data. 2A) Depicts a schematic of the Taxa4Meta analysis workflow. 2B) Depicts Spearman correlations for family abundances comparing simulated 16S data input (ground truth) with taxonomic output using different taxonomic profilers covering a range of 16S variable regions (FIGS. 11A-11C shows additional benchmarking results). 2C) Depicts hierarchical clustering of family abundance profiles (−log 2 transformation of average relative abundances) generated by different taxonomic profilers. Kruskal-Wallis (KW) test together with Benjamini and Hochberg (BH) correction was used to compare family abundances for each pipeline for each data type: *=p<0.05 false discovery rate (FDR). Shotgun metagenomic and 16S amplicon sequencing were performed on the same human stool DNA (N=27) using 454 pyrosequencing or Illumina platforms. 2D) Depicts how Taxa4Meta abundance profiles showed the highest similarity to shotgun WGS data (Kraken2). Abundance-weighted Jaccard distance was calculated between 16S profiler-specific outputs versus gold standard WGS (Kraken2). The most abundant 29 family features (totaling 0.95±0.07 (SD) of family abundance) across all analyses were used for visualization and benchmarking.

FIGS. 3A-3C, describes how pan-microbiome analysis identifies diarrheal disease-specific taxa. 3A) Depicts β-diversity analysis of Taxa4Meta collapsed species profiles (green ellipse, healthy-associated microbiome; red ellipse, Clostridium difficile Infection (CDI)-associated microbiome). ANOSIM testing was used to compare disease vs controls. Abundance-weighted Jaccard distance was used for β-diversity analysis. 3B) Depicts quantification of FIG. 3A, relative abundance of pathobiome (Enterococcus, Streptococcus, Clostridioides, Escherichia/Shigella, Klebsiella and Pseudomonas) was significantly elevated in Crohn's disease (CD) and CDI patients. ***=p<0.001 (pairwise Wilcoxon test with BH correction). ANOSIM test was performed with 999 permutations. Boxplots show interquartile range (IQR), median and whiskers extended to values <1.5×IQR from 1st and 3rd quartile, respectively. 3C) Depicts average family relative abundance of each disease group (FIG. 16 shows the top 21 abundant family abundances across datasets). *=p<0.05 (KW test with BH correction).

FIGS. 4A-4D, describes how Taxa4Meta mediated pan-microbiome profiling improves supervised classification. 4A) Depicts β-diversity analysis of Taxa4Meta collapsed species profiles for V1V3 and V3V5 amplicon data generated from the same DNA extracts²⁸ (control or functional gastrointestinal disorders (FGIDs)). n.s., not significant (pairwise Wilcoxon test with BH correction). 4B) depicts ROC analysis of supervised classification (random forest, (RF)) using 16S-region specific vs. pan-microbiome genera. The roc.test( ) function from pROC package was used to compare two ROC curves: **=p<0.01 (delong method). 4C) Depicts β-diversity analysis of multiple CDI cohorts (datasets #22-27) using collapsed species profiles generated by Taxa4Meta. ***=p<0.001 (pairwise Wilcoxon test with BH correction). 4D) Depicts improved cross-validation of CDI and Control subjects using pan-microbiome profiling of 454 and Illumina data. Ten iterations of random, stratified sub-sampling of training sets were performed. The random forest was used for supervised classification analysis. n.s., not significant (pairwise Wilcoxon test with BH correction). Area-under-the-curve (AUC); classification accuracy (CA). ANOSIM test was performed with 999 permutations. Stacking model of NB, RF and SVM learners was used for supervised classification analysis. Boxplots show interquartile range (IQR), median and whiskers extended to values <1.5×IQR from 1st and 3rd quartile, respectively.

FIGS. 5A-5C, describes supervised classification of chronic diarrheal diseases using pan-microbiome taxonomy. 5A) Depicts an outline of supervised classifications performed across clinical disease groups and matched controls. 5B) Depicts the top 10 species features ranked by random forest across five clinical groups. 5C) Depicts binary classification using Taxa4Meta collapsed species profiles. Individual or combined RF-ranked top Taxa4Meta 100 features were used for binary classification analysis. *=p<0.05; n.s., not significant (pairwise Wilcoxon test). Boxplots show interquartile range (IQR), median and whiskers extended to values <1.5×IQR from 1st and 3rd quartile, respectively.

FIGS. 6A-6B, describes a prototypical pan-microbiome diagnostic workflow for stratifying CDI, inflammatory bowel disease (IBD), and irritable bowel syndrome (IBS) patients. 6A) Depicts a scheme for diarrheal patient stratification using binary classification models. All collapsed species features were used for classification in the training process. 6B) Depicts independent cohort validation of CDI and IBD models. A binary threshold of 0.5 was applied for calculating disease classification accuracy. Cohort information is provided in Table 21. *=p<0.001 (pairwise Wilcoxon test with BH correction). Boxplots show interquartile range (IQR), median and whiskers extended to values <1.5×IQR from 1st and 3rd quartile, respectively.

FIGS. 7A-7C, describes the identification of region-specific optimal length ranges for clustering 16S amplicons. 7A) Depicts Spearman correlations of VSEARCH-based de novo clustering with 99% similarity for 16S V3V5 amplicons of variable length from the same parent 16S sequence. 7B) Depicts Spearman correlations of VSEARCH-based de novo clustering with 99% similarity for 16S V6V9 amplicons of variable length from the same parent 16S sequence. 7C) Depicts Spearman correlations of VSEARCH-based de novo clustering with 99% similarity for 16S V4 amplicons of variable length from the same parent 16S sequence. Orange box highlights variable length input reads used for sequence clustering by Taxa4Meta. *=p<0.05; **=p<0.01 (Wilcoxon test).

FIG. 8, describes how controlling the identity and coverage of sequence alignment minimizes taxonomic over-classification. Taxonomic annotation using the Bayesian LCA-based Taxonomic Classification (BLCA) tool was performed full-length amplicons simulated from unclassified (family level) 16S sequences of RDP database (release 11.5). Ten iterations of BLCA run were performed for 1% of unclassified sequences. Pairwise Wilcoxon test with Benjamini-Hochberg Procedure was used for any two group comparison. Over-classification rate is the classified proportion of BLCA annotation without considering the confidence score of taxonomic assignment. ***=p<0.001 (pairwise Wilcoxon test with BH correction).

FIGS. 9A-9D, describes how taxonomic accuracy is dependent on read length, sequence orientation, and variable 16S amplicon region. Confidence score of taxonomic assignment and the proportion of correct versus wrong annotated reads are plotted for 9A) V1V3, 9B) V3V5, 9C) V4, and 9D) V6V9 regions. Orange box highlights optimal length range for accurate sequence clustering using VSEARCH with 99% similarity. This sequence range was later applied in the Taxa4Meta workflow. Confidence scores are presented as mean±SEM. *=p<0.05 (Wilcoxon test).

FIGS. 10A-10B, describes how confidence thresholds for accurate taxonomic assignment were determined. Different confidence thresholds for taxonomic assignment using the BLCA tool at both genus 10A) and species 10B) rank were benchmarked by calculating the proportion of true positive (TP), false negative (FN), true negative (TN) and false positive (FP) annotations for combined variable length amplicons generated from NCBI 16S RefSeq database with known taxonomic lineage. TP and FP annotations are present while TN and FN annotations are absent in the final annotation output. Data are presented as mean±SEM.

FIGS. 11A-11C, describes the Benchmarking of Taxa4Meta and other pipelines using simulated amplicon data of variable lengths. 11A) Depicts hierarchical clustering of family abundance profiles (−log 2 transformation of relative abundance) generated by different analytic pipelines. NCBI 16S rRNA sequences were used for data simulation, but cutadapt failed to extract targeted regions of some sequences due to strict mapping of degenerate primers to full-length 16S sequence. Read count was randomly generated from 1 to 50 for each amplicon sequence (V1-V3, V3-V5, V4 and V6-V9) prior to length trimming; amplicons with variable lengths (suggested by Taxa4Meta core parameters) were concatenated for benchmarking different pipelines. Average total abundance at family level matched to ground truth is 0.95 with the standard deviation of 0.02 across all results from different pipelines. 11B) Depicts the percentage of undetected families by each pipeline for simulated amplicons of different 16S variable regions. 11C) Depicts relative abundance of C. difficile from simulated amplicon data analyzed by different pipelines. Only two strains of C. difficile were included in NCBI 16S rRNA sequence database, but V3-V5 amplicons of C. difficile were removed during data simulation because their amplicon lengths did not fall into the length range that was used (for more information, see Table 22).

FIGS. 12A-12D, describes how consistent Taxa4Meta-based genus and family abundance profiles are across different sequence strategies. 12A) Depicts total read counts from Taxa4Meta output (mean±SD; ***=p<0.001). 12B) Depicts relative family taxonomic abundance. 12C) Depicts relative abundance of shared and non-shared genera. 12D) Depicts Pearson correlations of the relative abundance of commonly shared genera across different 16S sequencing strategies. All correlations are significant (p<0.001).

FIG. 13, describes how Taxa4Meta analysis results in low error rates in species calls for 16S amplicon data. Species identified by MetaPhlAn2 were used as the reference since it has high precision in species identification. Bacterial tax_ids retrieved from NCBI TaxIdentifier tool for each species identified by different pipelines were used for mapping across taxonomic profiles since species names might be reclassified but tax_ids are preserved. ***=p<0.001 (pairwise Wilcoxon test with BH correction).

FIGS. 14A-14B, describes how meta-analysis controls of diarrheal microbiome datasets show three conventional gut enterotypes. Bray-Curtis dissimilarity metric with nonmetric multidimensional scaling (NMDS) was used in β-diversity analysis procedure for showing abundant taxonomic features at 14A) genus and 14B) family rank that drive sample clustering. ANOSIM analysis was performed with Bray-Curtis distance profile for pairwise comparison. Higher R values (>0.2) of ANOSIM test indicate more difference between groups. The 2D kernel density estimation of samples was measured by geom_density_2d from ggplot2 package and was showed as contours in NMDS ordination plot. The envfit function from vegan package was used for fitting taxonomic features (family relative abundance) to 2-dimensional NMDS ordination plot. ***=p<0.001 (Wilcoxon test).

FIGS. 15A-15B, depicts the Alpha-diversity indices of meta-analysis training datasets. 15A) Depicts Richness and Shannon index for each disease group. 15B) Depicts Richness and Shannon index for each dataset. Log 10 transformation was performed for richness measure from breakaway package. KW test was performed across groups or datasets of each sample group as indicated in each sub-plot. Data are presented as the median with first and third quartiles in the boxplot.

FIG. 16, depicts the taxonomic abundance of meta-analysis training datasets. Hierarchical clustering of family-level relative abundance profiles (−log 2 transformation of median relative abundance) of each sample group of each dataset. Kruskal-Wallis (KW) test together with Benjamini-Hochberg procedure was used to compare family-level relative abundance of sample groups of all dataset: *=p<0.05 (FDR). Only top 21 abundant family features totaling the average relative abundance of 0.93 (standard deviation of 0.095) across all samples were used for heatmap generation and clustering analysis.

FIG. 17, depicts the total relative abundance of classified and unclassified species across adult and pediatric datasets. Data are presented as mean±SD.

FIG. 18, depicts random forest-based feature ranking for pediatric FGID patients using individual or pan-microbiome data.

FIG. 19, describes how pan-microbiome analysis can identify metabolic pathways related to diarrheal disease-specific taxa and metagenomic functions. Depicted is a heatmap of PICRUSt2 pathway analysis showing disease-specific (UC. CDI, CD, Control, IBS) metabolic functions. Median abundance of selected differential pathways was plotted for each adult diarrheal disease (see FIGS. 23A-23E for top 20 differential pathways identified between each adult diarrheal disease and healthy controls.). *=p<0.05 (KW test with BH correction)

FIGS. 20A-20D, describes how gut enterotype clusters can impact the accuracy of supervised classification models. 20A) Depicts a bar graph depicting the relative abundance of five enterotype clusters in meta-analysis adult datasets. DMM was used for enterotyping analysis based on relative abundance profile at family level. 20B) Depicts the distribution of five enterotype clusters in five clinical groups (Control, IBS, UC, CD, and CDI). 20C) Depicts the workflow of random sub-sampling for supervised classification of enterotype-specific gut microbiome. Ninety samples from each clinical group were sampled to create training and validation datasets with the balanced sample count for further classification. 20D) Depicts independent validation of supervised classifiers (random forest) shown in panel C. Top 100 features ranked by random forest were used for classification (for additional details, see Tables 1-17). Each circle represents the overall classification accuracy of each non-trained subsampled dataset. Feature selection was not performed for this analysis. ***=p<0.001; n.s., not significant (pairwise Wilcoxon test with BH correction).

FIGS. 21A-21B, describes supervised classification of chronic diarrheal diseases using pan-microbiome taxonomic and functional-pathway features. 21A) Depicts a comprehensive classification analysis using individual or combined RF-ranked top 100 Taxa4Meta collapsed species and RF-ranked top 100 PICRUSt2 pathways. Statistical test was performed for data excluding results (as outlier) generated with NB learner: *=p<0.05; n.s., not significant (pairwise Wilcoxon test with BH correction). 21B) Depicts how binary classification using Taxa4Meta collapsed species profile demonstrates improved scores of AUC and CA when compared to PICRUSt2 pathway profile independent of different learners. Individual or combined RF-ranked top 100 features of Taxa4Meta and PICRUSt2 were used for binary classification analysis. *=p<0.05; n.s., not significant (Wilcoxon test).

FIG. 22, describes benchmarking of Taxa4Meta and other pipelines using simulated amplicon data of variable ranks. In particular, the figure depicts precision species calls on C. difficile amplicons is associated with 16S variable regions, sequence length and orientation. C. difficile 16S rRNA gene sequences collected from SILVA, RDP and GG databases were confirmed by BLASTN prior to amplicon simulation.

FIGS. 23A-23E, describes the identification of differential metabolic pathways in chronic diarrheal diseases. 23A through 23D) Depict LDA scores of the top 20 Random Forest (RF)-ranked pathways between two adult groups for 23A) CDI vs Control, 23B) CD vs Control, 23C) UC vs Control, 23D) IBS vs Control. 23E) Depicts a heatmap of differential pathways (from A-D) across adult diarrheal patients. Pink box highlights important pathways that can differentiate diarrheal diseases.

FIG. 24, describes AUC and CA values of binary classification models using Taxa4Meta collapsed species and PICRUSt2 pathway profiles. *=p<0.05; n.s., not significant (pairwise Wilcoxon test with BH correction).

### DETAILED DESCRIPTION

Described herein are methods and compositions suitable for the treatment of disorders associated with dysbiosis of the microbiome. Use of the one or more compositions may be employed based on methods described herein. Methods and/or compositions described herein may be included as components of one or more kits suitable for treatment of disorders associated with dysbiosis of the microbiome. Other embodiments are discussed throughout this application. Any embodiment discussed with respect to one aspect of the disclosure applies to other aspects of the disclosure as well and vice versa. The embodiments in the Example section are understood to be embodiments that are applicable to all aspects of the technology described herein.

The use of the word “a” or “an” when used in conjunction with the term “comprising” in the claims and/or the specification may mean “one.” but it is also consistent with the meaning of “one or more.” “at least one.” and “one or more than one.”

As used herein, the term “about” or “approximately” refers to a quantity, level, value, number, frequency, percentage, dimension, size, amount, weight or length that varies by as much as 30, 25, 20, 25, 10, 9, 8, 7, 6, 5, 4, 3, 2 or 1% to a reference quantity, level, value, number, frequency, percentage, dimension, size, amount, weight or length. In particular embodiments, the terms “about” or “approximately” when preceding a numerical value indicates the value plus or minus a range of 15%, 10%, 5%, or 1%. With respect to biological systems or processes, the term can mean within an order of magnitude, preferably within 5-fold, and more preferably within 2-fold, of a value. Unless otherwise stated, the term ‘about’ means within an acceptable error range for the particular value.

The use of the term “or” in the claims is used to mean “and/or” unless explicitly indicated to refer to alternatives only or the alternatives are mutually exclusive, although the disclosure supports a definition that refers to only alternatives and “and/or.” It is also contemplated that anything listed using the term “or” may also be specifically excluded.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “Antimicrobial” as used herein is a general term for drugs, chemicals, or other substances that either kill or slow the growth of microbes. Among the antimicrobial agents are antibacterial drugs, antiviral agents, antifungal agents, and antiparasitic drugs. In patients this includes drugs and/or treatment that impacts microbiome community composition.

As used herein, the terms “arrays”, “microarrays”, and “DNA chips” refer to an array of distinct oligonucleotides affixed to a substrate, such as glass, plastic, paper, nylon or other type of membrane, filter, chip, or any other suitable solid support. The polynucleotides can be synthesized directly on the substrate, or synthesized separate from the substrate and then affixed to the substrate. The oligonucleotides on the array may be designed to bind or hybridize to specific nucleic acids, such as a specific SNP or a specific CNV, for example.

The terms “Clostridioides difficile infection” “C. difficile infection” or “CDI” as used herein refers to an individual that has presence of Clostridioides difficile in their body to an extent and under conditions in which a sufficient level of toxins from the Clostridioides difficile results in diarrhea. This is in contrast to presence of Clostridioides difficile in an individual that is considered a carrier for the bacteria and that has no diarrhea.

The term “classifier” as used herein refers to an algorithm that implements a disease classification, notably CDI, IBS, IBD UC, and/or IBD CD diagnosis, or CDI, IBS, IBD UC, and/or IBD CD risk, or risk of C. difficile colonization. In other embodiments, the term refers to an algorithm that implements a disease classification for diagnosis or risk or risk of colonization for one or more pathogens other than C. difficile.

The term “feature” as used herein refers to a microbe, biological molecule, and/or metabolic pathway that is representative of a detectable difference between a control or reference standard and the corresponding microbe, biological molecule, and/or metabolic pathway in an individual with or at risk of developing CDI, IBS, IBD UC, and/or IBD CD. A feature may be the presence, absence, and/or levels of a microbe, nucleic acid sequence (such as 16S rRNA), protein, small molecule, metabolic pathway, and/or a combination thereof.

The term “pan-microbiome” as used herein refers to a composite of two or microbiomes, for example, a composite of two or more data sets reflective of two or more microbiomes. A pan-microbiome may be larger than any single microbial community of an individual or a group. In some embodiments, a pan-microbiome includes two or more populations, two or more demographics, and/or data collected through two or more acquisition methodologies.

As used herein, the term “oligonucleotide” refers to a short chain of nucleic acids, cither RNA, DNA, and/or PNA. The length of the oligonucleotide could be less than 10 base pairs, or at minimum or no more than 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49,50, 51, 52, 53, 54, 55, 56, 57, 58, 59, 60, 61, 62, 63, 64, 65, 66, 67, 68, 69, 70, 71, 72, 73, 74, or 75 base pairs. The oligonucleotide can be synthesized using by methods including phosphodiester synthesis, phosphotriester synthesis, phosphite triester synthesis, phosphoramidite synthesis, solid support synthesis, in vitro transcription, or any other method known in the art.

As used herein, the term “PCR primer” refers to an oligonucleotide that is used to amplify a strand of nucleic acid in a polymerase chain reaction (PCR). Primers may have 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 89%, 90%, 91%, 92%, 93%, 94%, 95%, 96%, 97%, 98%, 99%, or 100% homology to the template the primers hybridize to, wherein the 3′ nucleotide of the primer is complementary to the template. In some embodiments, lower annealing temperatures are used for initial cycles, for example cycles 1, 2, 3, 4, and/or 5, of the reaction.

“Treatment,” “treat,” or “treating” means a method of reducing the effects of a disease or condition. Treatment can also refer to a method of reducing the disease or condition itself rather than just the symptoms. The treatment can be any reduction from pre-treatment levels and can be but is not limited to the complete ablation of the disease, condition, or the symptoms of the disease or condition. Therefore, in the disclosed methods, treatment” can refer to a 10%, 20%, 30%, 40%, 50%, 60%, 70%, 80%, 90%, or 100% reduction in the severity of an established disease or the disease progression, including reduction in the severity of at least one symptom of the disease. For example, a disclosed method for reducing the immunogenicity of cells is considered to be a treatment if there is a detectable reduction in the immunogenicity of cells when compared to pre-treatment levels in the same subject or control subjects. Thus, the reduction can be a 10, 20, 30, 40, 50, 60, 70, 80, 90, 100%, or any amount of reduction in between as compared to native or control levels. It is understood and herein contemplated that “treatment” does not necessarily refer to a cure of the disease or condition, but an improvement in the outlook of a disease or condition. In specific embodiments, treatment refers to the lessening in severity or extent of at least one symptom and may alternatively or in addition refer to a delay in the onset of at least one symptom.

“Subject” may refer to an organism that comprises a microbiome. In certain embodiments, it refers to a human patient. In certain embodiments, it refers to an animal.

Microbiome science is a rapidly evolving field. In particular, advances in sequencing strategies and bioinformatics are facilitating improvements in stake holder's understanding of host-microbiota interactions¹⁻³. Nevertheless, these technological advances should not dissuade the use of retrospective microbiome data for development of methods of diagnosis, methods of treatment, and/or development of treatment compositions. The scientific community places great value on prior sequencing efforts that have explored microbiome community dynamics in human pathogenesis^{4,5,20,22}, because these projects could collectively provide critical insight into disease-specific associations. However, individual clinical microbiome surveys often utilize cohort-specific sequencing platforms, 16S primer regions and bioinformatics pipelines, which the inventors have systematically demonstrated herein, requires a consolidated bioinformatics approach to mitigate technological and demographic bias, as well as taxonomic misclassification. The findings presented herein show that previous microbiome meta-analyses have not adequately addressed these limitations^{20,22,23}. To address this, the inventors designed Taxa4Meta, a bioinformatics pipeline for accurate taxonomic profiling after systematically benchmarking sequence orientation and length so that data output can be reliably utilized from different 16S variable regions. Given the challenges of accurately merging OTU/ASV tables generated from different 16S variable regions, the inventors show that collapsed taxonomic annotations of Taxa4Meta OTU profiles offer a valuable new binning approach to facilitate meta-analysis of diverse 16S amplicon data.

Supervised classification represents an important downstream application of clinical microbiome surveys for development of diagnostic pipelines and the prescribing of subsequent treatment regimens, especially for gastrointestinal diseases where altered community dynamics are reported^{4,5,18,19,21,24-26}. Generally, diagnostic workflows require construction of large curated databases to facilitate cohort-specific classifier training and cross-validation of disease-specific biomarkers. Population-scale meta-analysis represents an attractive approach to adequately power microbiome surveys for disease classification because of the need to control for large variations in human genetics and demographics², as well as the technology bias¹² that contributes to false discovery rates. Notably, when applying the Taxa4Meta pipeline to identical DNA extracts sequenced using different strategies, as shown herein, the inventors have identified several prominent disease classification limitations due to this bias. To compensate for these technological hurdles, the inventors have developed a pan-microbiome profiling concept that achieves superior disease classification accuracy.

Taken together, the inventors have addressed a significant bioinformatics challenge using a new workflow (Taxa4Meta) developed for accurate sequence clustering and taxonomic annotation across multiple 16S regions. As described herein, Taxa4Meta was applied to comprehensively re-analyze diverse 16S datasets generated from multiple retrospective gastrointestinal disease cohorts investigated across four continents. Collapsed species abundance for each 16S dataset were successfully combined for downstream microbiome interpretation and supervised classification of diarrheal patients who are difficult to diagnose because of overlapping gastrointestinal symptoms. This improved classification of diarrheal patients can be leveraged for improved methods of patient treatment, and/or identification of compositions for treatment of the underlying causes of dysbiosis. The “best practices” approach disclosed herein facilitated construction of a prototypic diagnostic workflow based on disease-specific pan-microbiome biomarkers.

Other objects, features and advantages of the present disclosure will become apparent from the following detailed description. It should be understood, however, that the detailed description and the specific examples, while indicating specific embodiments, are given by way of illustration only, since various changes and modifications within the spirit and scope of the immediate disclosure will become apparent to those skilled in the art from this detailed description

I. Diseases Associated with Microbiome Dysbiosis

The human gut microbiome comprises bacteria, viruses, and fungi ideally living symbiotically with their human host. Individual species and collective bacterial functions within the gut microbiome confer many benefits throughout life including metabolizing dietary contributions, educating the immune system, defending against pathogens, and contributing to overall health and optimal growth. The gut microbiome is affected by and influences pathologies including but not limited to inflammatory bowel disease (IBD; both ulcerative colitis (UC) and Crohn's disease (CD)), Clostridium difficile infection (CDI), and irritable bowel syndrome (IBS).

A. Clostridium difficile infection (CDI)

Clostridium difficile is a bacterium that causes an infection (CDI) of the large intestine (colon). Symptoms can range from diarrhea to life-threatening damage to the colon. The bacterium is often referred to as C. difficile or C. diff. Illness from C. difficile typically occurs after use of antibiotic medications. It most commonly affects older adults in hospitals or in long-term care facilities. In the United States, about 200,000 people are infected annually with C. difficile in a hospital or care setting. Thankfully these numbers are trending lower than in previous years because of improved prevention measures. People not in care settings or hospitals also can develop C. difficile infection. Some strains of the bacterium in the general population may cause serious infections or are more likely to affect younger people. In the United States, about 170,000 infections occur annually outside of health care settings, and worryingly these numbers are increasing. Some people carry C. difficile bacteria in their intestines but never become sick. These individuals are carriers of the bacteria and may spread infections. Signs and symptoms usually develop within 5 to 10 days after starting a course of antibiotics. However, they may occur as soon as the first day or up to three months later. The most common signs and symptoms of mild to moderate C. difficile infection are: watery diarrhea three or more times a day for more than one day and/or mild abdominal cramping and tenderness. People who have a severe C. difficile infection tend to become dehydrated and may need to be hospitalized. C. difficile can cause the colon to become inflamed and sometimes form patches of raw tissue that can bleed or produce pus. Signs and symptoms of severe infection include: watery diarrhea as often as 10 to 15 times a day, abdominal cramping and pain (which may be severe), rapid heart rate, dehydration, fever, nausea, increased white blood cell count, kidney failure, loss of appetite, swollen abdomen, weight loss, and/or blood or pus in the stool. C. difficile infection that is severe and sudden, an uncommon condition, may also cause intestinal inflammation leading to enlargement of the colon (also called toxic megacolon) and sepsis. Sepsis is a life-threatening condition that occurs when the body's response to an infection damages its own tissues. People who have these conditions are generally admitted to an intensive care unit.

C. difficile bacteria enter the body through the mouth. They can begin reproducing in the small intestine. When they reach the large intestine (colon), they can release tissue-damaging toxins. These toxins destroy cells, produce patches of inflammatory cells and cellular debris, and cause watery diarrhea. When the bacteria are outside the colon, virtually anywhere in the environment, they are in a dormant state, or essentially quiescent. This enables them to survive for a long time in any number of places, including but not limited to human or animal feces, surfaces in a room, unwashed hands, soil, water, and/or food. When bacteria once again find their way into a person's digestive system, they begin to produce infection again. The ability of dormant C. difficile to survive outside the body enables the generally easy transmission of the bacterium, particularly in the absence of thorough hand-washing and cleaning.

Risk factors associated with developing a C. difficile infection include but are not limited to, taking antibiotics or other medications such as Clindamycin, Cephalosporins, Penicillin's, Fluoroquinolones, and/or potentially certain proton pump inhibitors. The majority of C. difficile infections occur in people who are or who have recently been in a health care setting, including hospitals, nursing homes and long-term care facilities, where germs spread easily, antibiotic use is common and people are especially vulnerable to infection. Additionally, certain medical conditions or procedures may increase an individual's susceptibility to a C. difficile infection, such as IBS, a weakened immune system from a medical condition or treatment (e.g., chemotherapy), chronic kidney disease, a gastrointestinal procedure, and/or other abdominal surgery. Additionally, age is a major risk factor for CDI infection.

Complications associated with C. difficile infection include but are not limited to: dehydration, kidney failure, toxic megacolon, bowel perforation, and/or death. The necessity for swift and correct diagnosis and initiation of appropriate therapeutic interventions are key steps in limiting the impact of this potentially devastating disease. Methods and compositions disclosed facilitate this process.

### B. Irritable Bowel Syndrome (IBS)

Irritable bowel syndrome (IBS) is a common disorder that affects the large intestine. Signs and symptoms include cramping, abdominal pain, bloating, gas, and diarrhea or constipation, or both. IBS is a chronic condition that will require long term management. Only a small number of people with IBS have severe signs and symptoms. Some people can control their symptoms by managing diet, lifestyle and stress. More-severe symptoms can be treated with medication and counseling.

The signs and symptoms of IBS vary but are usually present for a long time. The most common include: abdominal pain, cramping or bloating that is related to passing a bowel movement, changes in appearance of bowel movement, changes in how often you are having a bowel movement, and/or other symptoms that are often related include bloating, increased gas or mucus in the stool. Certain severe symptoms associated with IBS may include: weight loss, diarrhea at night, rectal bleeding, iron deficiency anemia, unexplained vomiting, difficulty swallowing, and/or persistent paint hat isn't relieved by passing gas or a bowel movement.

The precise cause of IBS is not yet known. But factors that appear to play a role in IBS disease progression include: muscle contractions of the intestine that are stronger and/or last longer than normal and/or weaker than normal, poor nervous system signaling, severe infection, early life stress, and/or changes in the gut microbiome. IBS symptom “flares” can be triggered by certain foods such as beverages, wheat, dairy, citrus fruits, beans, cabbage, milk and/or carbonated drinks, or stress.

Risk factors associated with IBS include being young, being female, having a family history of IBS, and/or having anxiety, depression and/or other mental health issues.

Major complications associated with IBS include chronic constipation or diarrhea that can cause hemorrhoids, a reduction in the quality of life, and exacerbation of mood disorders.

Correct diagnosis and management of IBS is an essential step in long term management of the disease. Methods and compositions disclosed facilitate this process.

### C. Inflammatory Bowel Disease (IBD)

Inflammatory bowel disease (IBD) is an umbrella term used to describe disorders that involve chronic inflammation of your digestive tract. The two major types of IBD include Ulcerative colitis (UC) which involves inflammation and development of ulcers along the superficial lining of the large intestine and rectum; and Crohn's disease (CD) which is characterized by inflammation of the lining of the digestive tract which can also involve the deeper layers of the digestive tract. Both ulcerative colitis and Crohn's disease usually are characterized by diarrhea, rectal bleeding, abdominal pain, fatigue and weight loss. IBD can be debilitating, and can sometimes lead to life-threatening complications.

Symptoms of IBD vary depending on the severity of the associated inflammation, and where in the digestive tract it occurs. Symptoms may range from mild to severe and may be interrupted by periods of remission. Symptoms common to both IBD UC and IBD CD include but are not limited to diarrhea, fatigue, abdominal pain and cramping, blood in the stool, reduced appetite, and/or unintended weight loss.

The exact causes of IBD remain elusive. However, it is thought that diet and stress may be involved, but perhaps these factors just aggravate the disease and are not the root cause. It is also thought that an immune system malfunction may also contribute to IBD development. When the immune system tries to fight off an invading virus or bacterium, an abnormal immune response causes the immune system to attack the cells in the digestive tract, too. Heredity also may play a role in that IBD is more common in people who have family members with the disease. However, most people with IBD don't have this family history.

Risk factors for development of IBD include but are not limited to age, race and/or ethnicity, family history, cigarette smoking and/or nonsteroidal anti-inflammatory medications (e.g., ibuprofen, naproxen sodium, etc.).

Complications associated with UC and/or CD include colon cancer, skin/eye/joint inflammation, medication side effects, primary sclerosing cholangitis, blood clots, bowel obstruction, malnutrition, fistulas, anal fissures, toxic megacolon, severe dehydration and/or perforation of the colon.

Correct diagnosis and management of IBD be it UC or CD is an essential step in long term management of the disease. Methods and compositions disclosed facilitate this process.

### II. Methods of Identifying Features, Evaluating Levels of Features, and Classifying Disease State

Disclosed herein are methods of identifying features, and evaluating the presence, absence, or levels of said features in a sample. In certain embodiments, a feature may also be described as a biomarker. In certain embodiments, one or more features are used to classify (e.g., diagnose) a disease state and/or identify one or more effective treatment options for a patient with an intestinal disorder characterized by microbiome dysbiosis (e.g., CDI, IBS, IBD UC, and/or IBD CD). In some embodiments, one or more features are used to diagnose a disease state and/or identify one or more effective treatment options for a patient with an intestinal disorder characterized by diarrhea.

In certain embodiments, a feature is a taxonomical classification. In certain embodiments, a feature is the presence, absence, or level of one or more microbial taxonomic units (e.g., genera, species, etc.). In certain embodiments, a feature is a metabolic pathway. In some embodiments, disclosed herein are methods of using a pan-microbiome profiling pipeline as a method suitable for identification of certain core features that can be used for accurate downstream diagnosis, accurate method of treatment prescription, and/or treatment composition determination.

It is contemplated that features may be evaluated based on one or more associated gene products. In some embodiments, a gene product is an amplicon complementary to at least a portion of a gene. In some embodiments, a gene product is an RNA transcript. In some embodiments, a gene product is a structural and/or functional RNA transcript. In some embodiments, a gene product is a protein expressed by an RNA transcript. In some embodiments, a gene product is a metabolic pathway associated with expression of a number of gene products. In some embodiments, a gene product is a metabolic pathway associated with expression of a number of gene products from a number of different species.

In some embodiments, features are identified using a pan-microbiome approach. In some embodiments, utilization of a pan-microbiome approach to identify features can reduce technical and/or demographic bias. In some embodiments, a pan-microbiome approach is a method that identifies and selects classifier features by analysis of microbiome data generated from two or more different sequencing strategies (e.g., 16S sequencing strategies) and/or two or more populations (e.g., two or more demographically distinct populations). Examples of pan-microbiome approaches are described herein, non-limiting examples of data sets suitable for use in a pan-microbiome approach are listed in Table 21.

In certain embodiments, a meta-analysis to determine the presence, absence, levels, expression, and/or activity of one or more features disclosed herein for correlation with a disease state can be performed. In statistics, a meta-analysis combines the results of several studies that address a set of related research hypotheses. This is normally done by identification of a common measure of effect size, which is modeled using a form of meta-regression. Generally, three types of models can be distinguished in the literature on meta-analysis: simple regression, fixed effects meta-regression and random effects meta-regression. Resulting overall averages when controlling for study characteristics can be considered meta-effect sizes, which are more powerful estimates of the true effect size than those derived in a single study under a given single set of assumptions and conditions. A meta-gene expression value, in this context, is to be understood as being the median of the normalized expression of a marker gene or activity. Normalization of the expression of a marker gene is preferably achieved by dividing the expression level of the individual marker gene to be normalized by the respective individual median expression of this marker genes, wherein said median expression is preferably calculated from multiple measurements of the respective gene in a sufficiently large cohort of test individuals. In some embodiments, a test cohort comprises at least 3, 10, 100, 200, 1000 individuals or more including all values and ranges thereof. In some embodiments, dataset-specific bias can be removed or minimized allowing multiple datasets to be combined for meta-analyses (Sec Sims et al. BMC Medical Genomics (1:42), 1-14, 2008, which is incorporated herein by reference in its entirety). In some embodiments, a meta-analysis cohort comprises the combination of 3, 6, 9, 12, 15, 18, 21, 24, 27, 30, 33, 36, 39, 42, 45 test cohorts or more including all values and ranges thereof.

### A. Identification of Features Suitable for Classification of Disease State

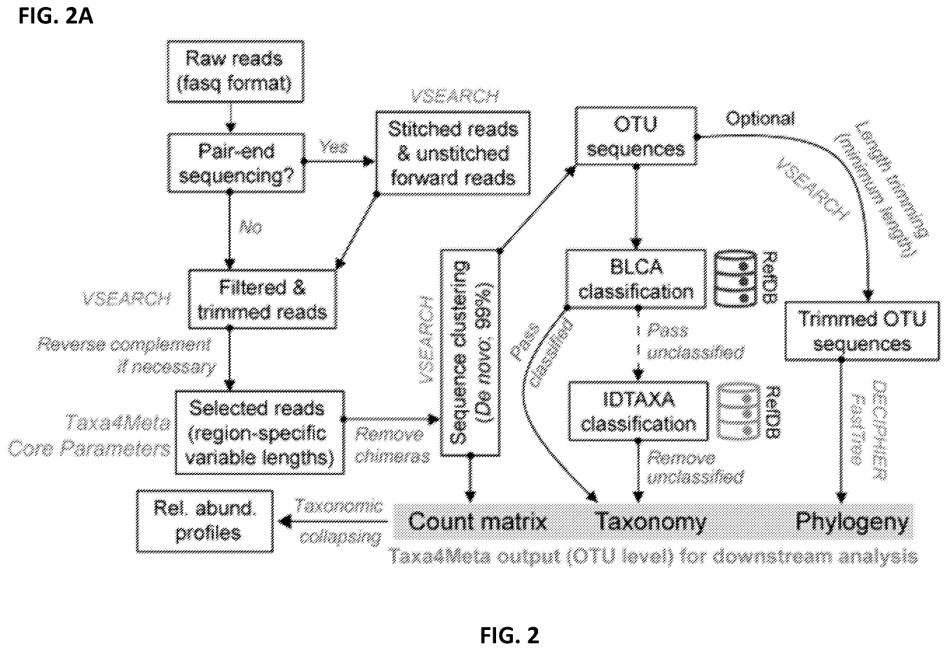

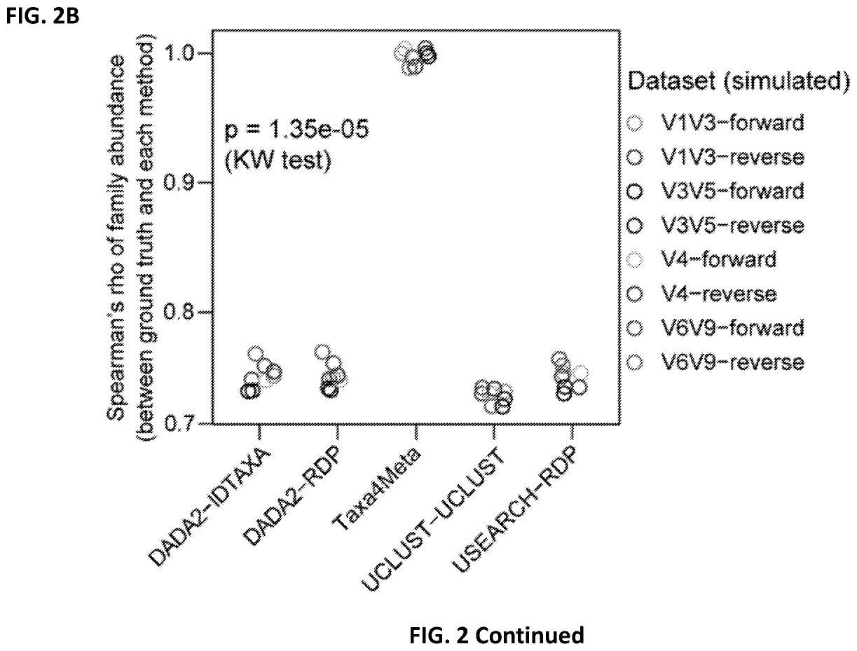

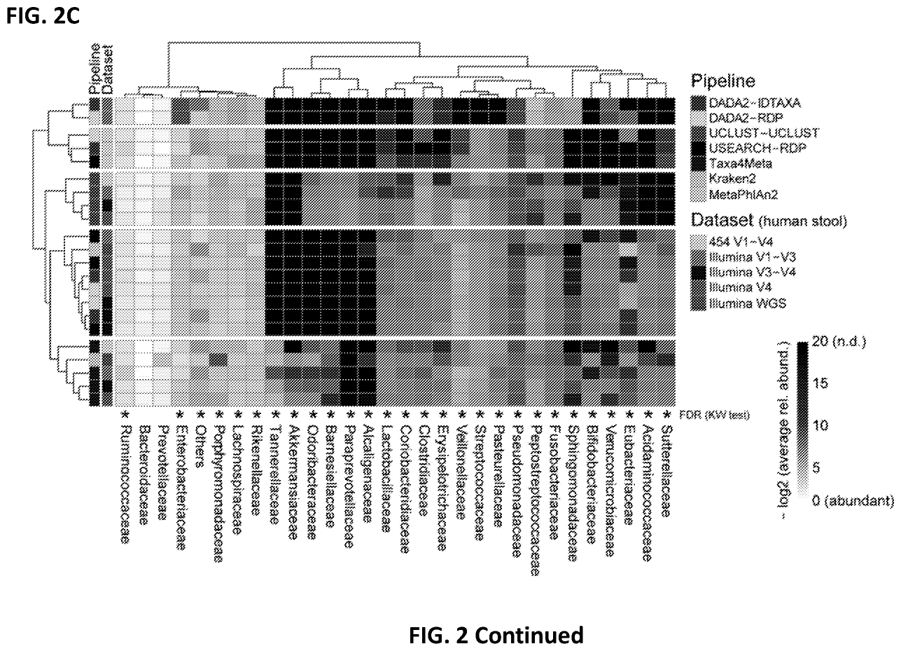

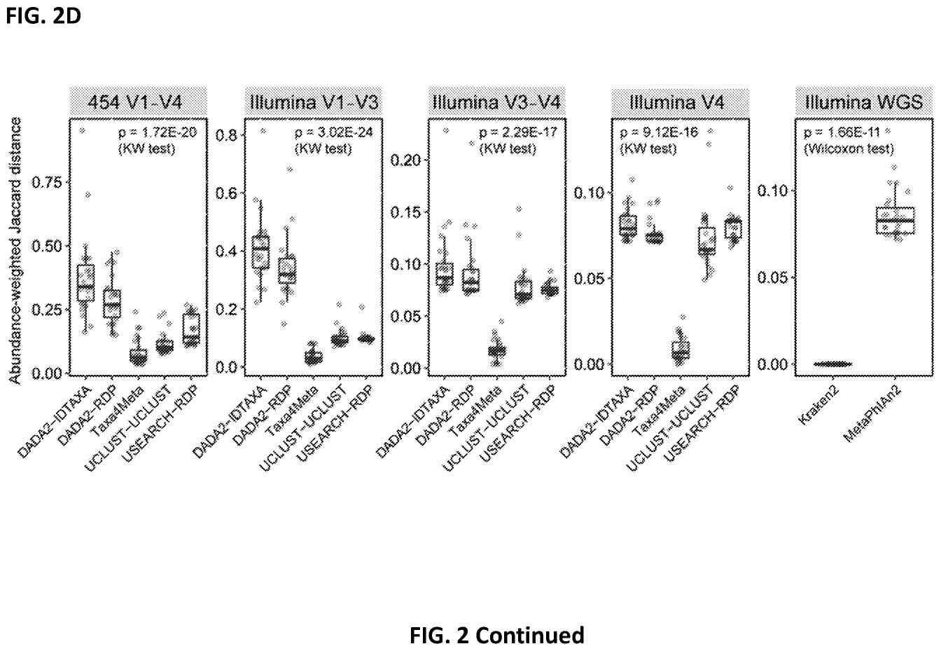

In certain embodiments, determination of features suitable for classification of microbiome dysbiosis disease state and subsequent methods stemming therefrom occurs as represented in FIG. 2.

In some embodiments, a step in determining features suitable for classification of microbiome dysbiosis associated disease state and/or determination of appropriate treatment methods comprises simulation of full-length and/or region-specific 16S amplicon data. In certain embodiments, simulation of full-length and/or region-specific 16S amplicon data can be based on reference data bases (e.g., NCBI 16S rRNA RefSeq database (downloaded in July 2019), Ribosomal Database Project (RDP) database (release 11.5)²⁸, etc.). In certain embodiments, bioinformatics tools such as cutadapt (version 2.4)²⁹ can be used to extract sequence fragments as full-length amplicons of targeted 16S variable regions (V1-V3, V3-V5, V4 and V6-V9) based on the forward and reverse primers (e.g., primers as listed in Table 22). In some embodiments, an error rate is permitted during sequence extraction, for example, an error rate of 0.05, 0.1, 0.15, 0.2, 0.25, etc. In certain embodiments, an error rate of 0.2 is permitted during sequence extraction. In certain embodiments, sequence length trimming and/or random simulation of sequence abundance and quality scores are performed for specific benchmarking purposes.

In some embodiments, a step in determining features suitable for classification of microbiome dysbiosis associated disease state and/or determination of appropriate treatment methods comprises benchmarking of sequence clustering and denoising using simulated amplicons, optionally with variable length. In certain embodiments, when benchmarking the accuracy of clustering or denoising for amplicon data using variable sequence lengths, random count ranging from 1 to 50 (e.g., 1, 2, 3, 4, 5 . . . 45, 46, 47, 48, 49, or 50) can be assigned for one or more or all parent full-length amplicons extracted from a reference database (e.g., NCBI 16S rRNA RefSeq sequences). In some embodiments, sequencing data may be generated in the reverse orientation and/or the forward orientation. In some embodiments, traditional 454 data is generated from reverse orientation, and length trimming from either forward or reverse orientation is applied to one or more, or each type of amplicon data. In some embodiments, length trimming results in 100, 150, 170, 200, 250, 300, 350, 400 and/or 450 bases for variable regions. e.g., V1-V3, V3-V5 and V6-V9 amplicon data. In some embodiments, length trimming results in 100, 150, 170, 200 and/or 250 bases for variable regions, e.g., V4 amplicon data. In some embodiments, random phred quality score (ASCII_BASE=33) ranging from 30 to 42 can be assigned to each base for sequencing denoising. In some embodiments, simulated amplicons of each sequence length represents one sample. In some embodiments, one or more or all samples with the same sequence orientation from the same 16S region can then be included for closed-reference or de novo clustering (e.g., using UCLUST (v1.2.22)³⁰ or VSEARCH (v2.9)³¹ or denoising using DADA2 (v1.8)³²). In some embodiments, sequence similarity thresholds including 0.97, 0.99 and 1.00 can be evaluated for each clustering strategy. In some embodiments, databases (e.g., the SILVA database (release 132)) can be used for closed-reference OTU picking. In some embodiments, simulated amplicons of variable length originating from the same parent full-length amplicon have the same sequence counts, in such situations, pairwise Spearman correlation analysis can be performed for sequence counts of any two sequence lengths (as two independent samples) in one or more OTU count tables

In some embodiments, a step in determining features suitable for classification of microbiome dysbiosis associated disease state and/or determination of appropriate treatment methods comprises benchmarking of taxonomic over-classification. In some embodiments, taxonomic over-classification for short amplicon data represents an important criteria for controlling false positives. In some embodiments, using default parameters in the Bayesian-based Lowest Common Ancestor (BLCA) tool¹¹ and its default database of NCBI 16S rRNA RefSeq can be used to annotate random and repeat sequences that were previously generated for benchmarking IDTAXA and other annotation tools¹⁰. In some embodiments, full-length 16S amplicons of unannotated sequences (e.g., at least down to family rank) are extracted from a suitable database (e.g., RDP database (release 11.5)) and are used for testing BLCA. In certain embodiments, BLASTN search of unannotated sequences against a suitable reference database (e.g., NCBI 16S rRNA RefSeq database) can be used to confirm that no best hits are identified at 97% threshold applied to either or both sequence identity and coverage. In some embodiments, simulated amplicons of unannotated RDP sequences are tested using different thresholds of sequence coverage and identity (e.g., ranging from 0.85 to 1.00 in BLCA). In some embodiments, 5, 6, 7, 8, 9, 10, 11, 12, 13, 14, 15, or more iterations of random sub-sampling (e.g., 1%, 2%, 3%, 4%, 5%) and BLCA annotation on those unannotated amplicons are performed for statistical determination of optimal sequence coverage and identity required for BLCA. In some embodiments, ten iterations of random sub-sampling (1%) and BLCA annotation on those unannotated amplicons are performed for statistical determination of optimal sequence coverage and identity required for BLCA. In some embodiments, taxonomic over-classification rate is defined as the classifiable proportion of unannotated amplicons at species level. In some embodiments, the confidence score of taxonomic assignment is not considered at this stage.

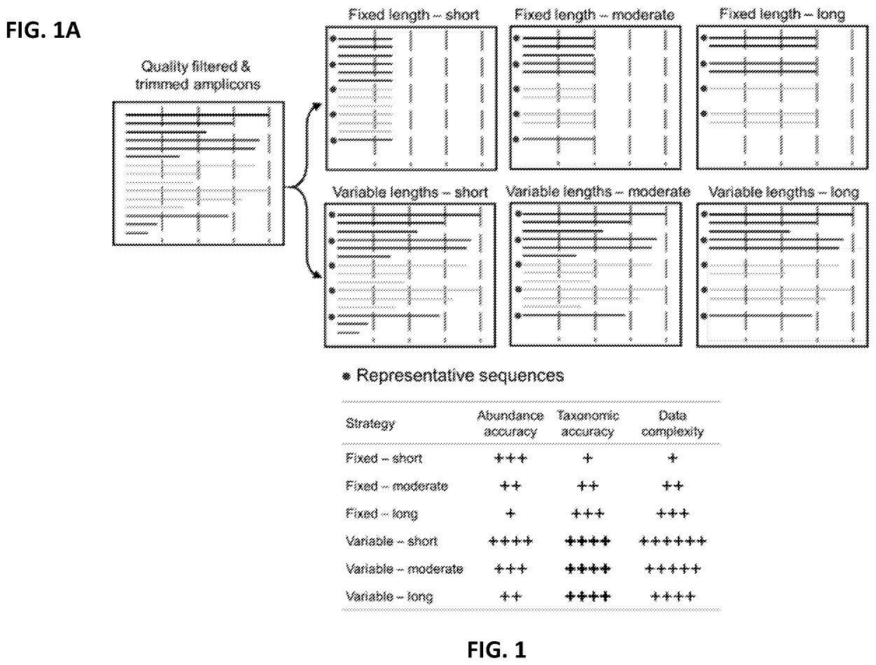

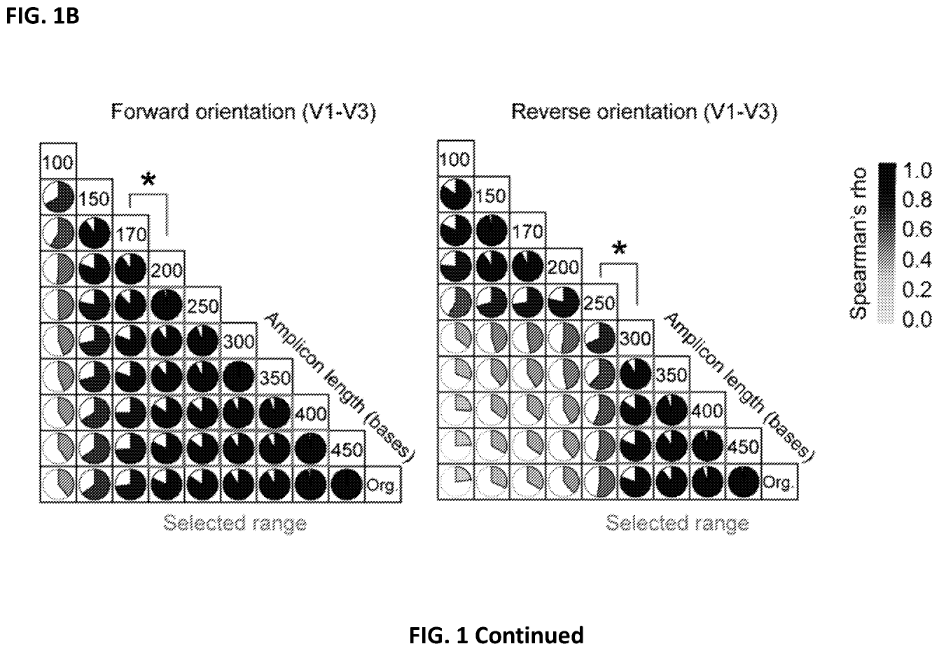

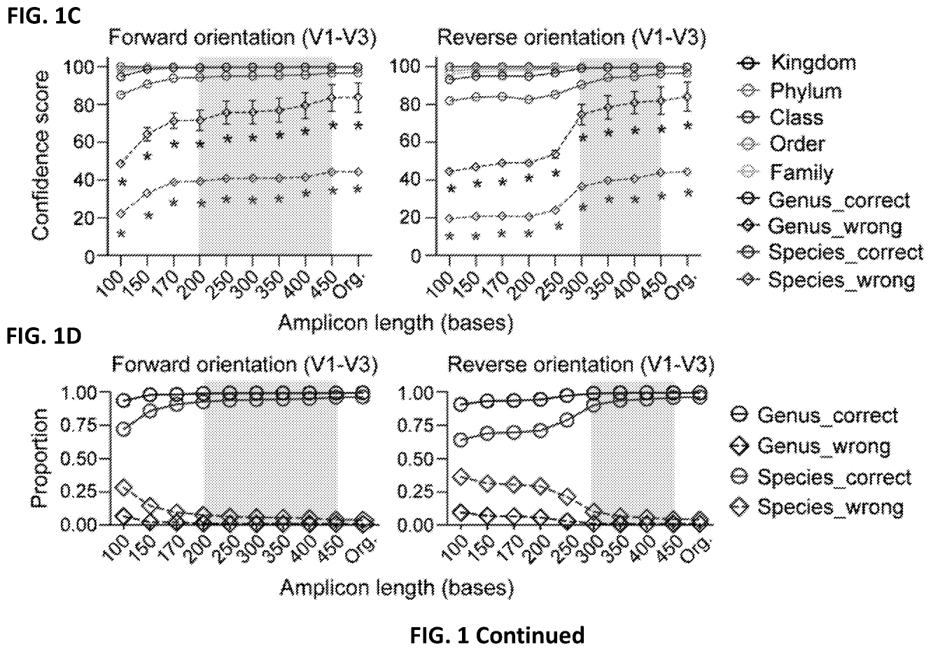

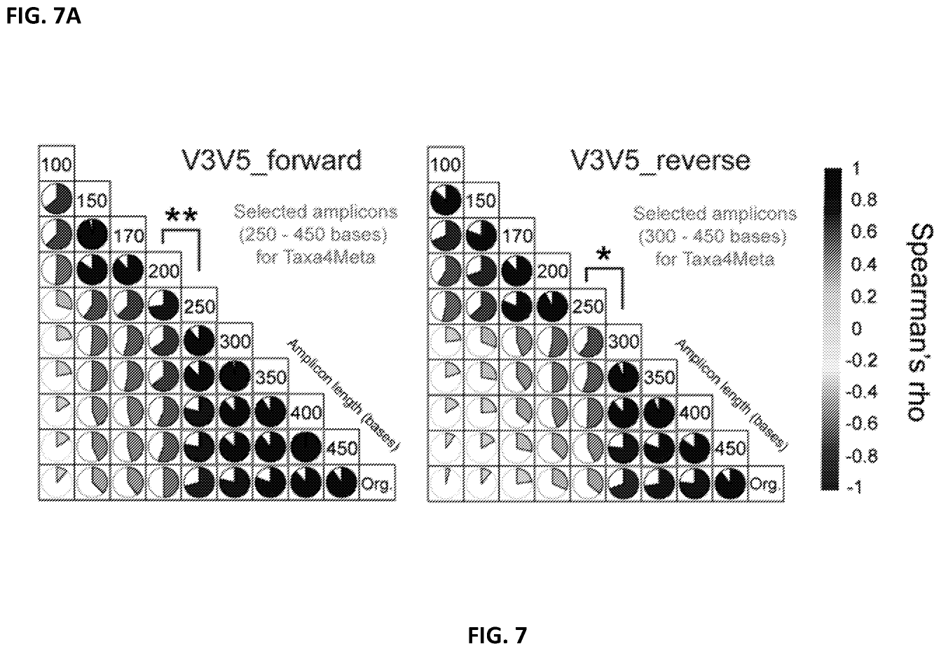

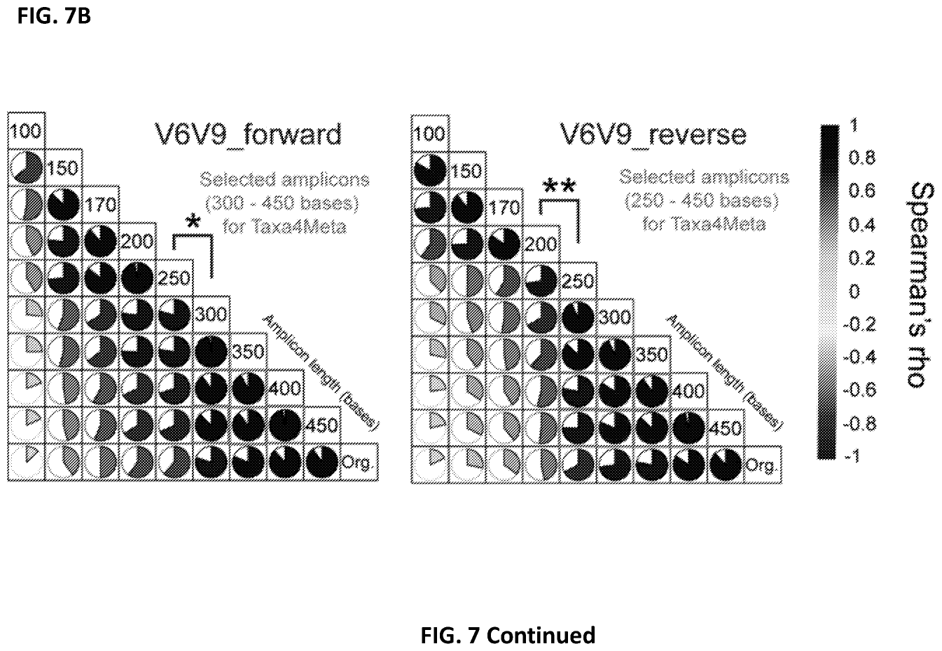

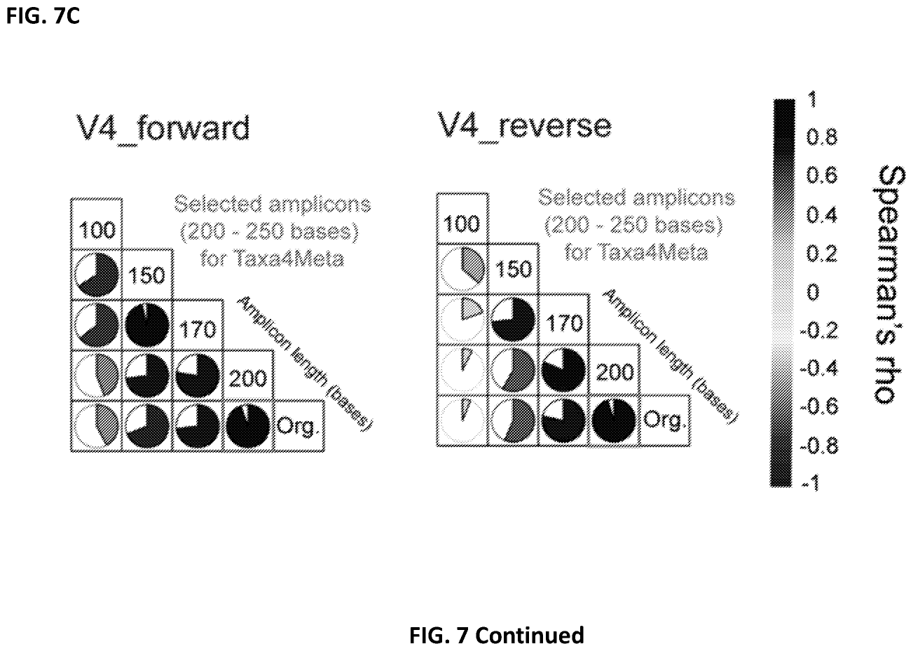

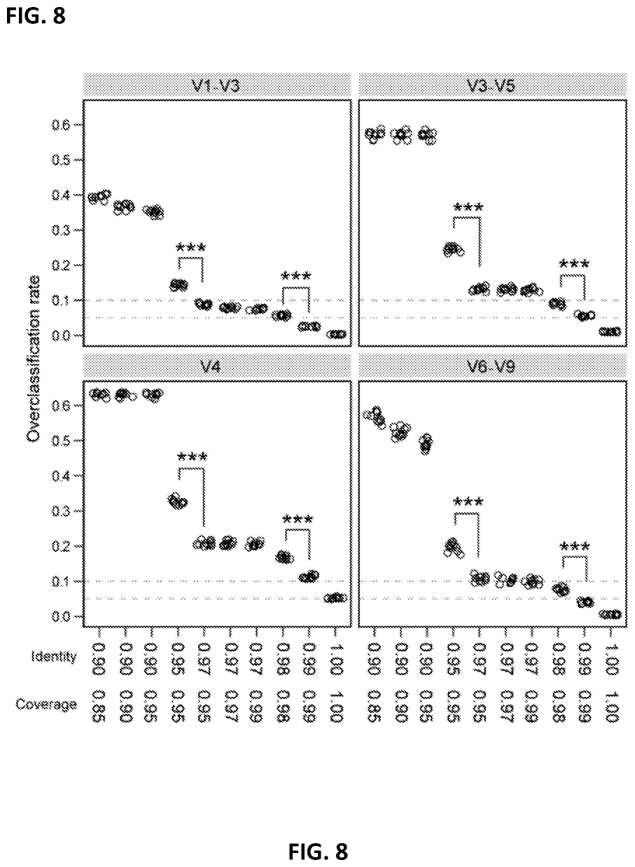

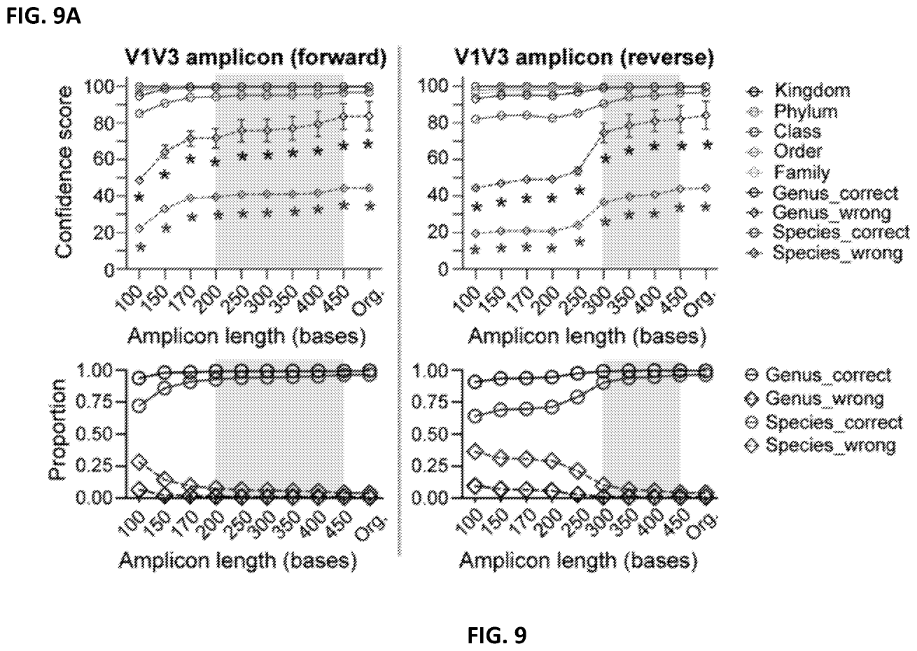

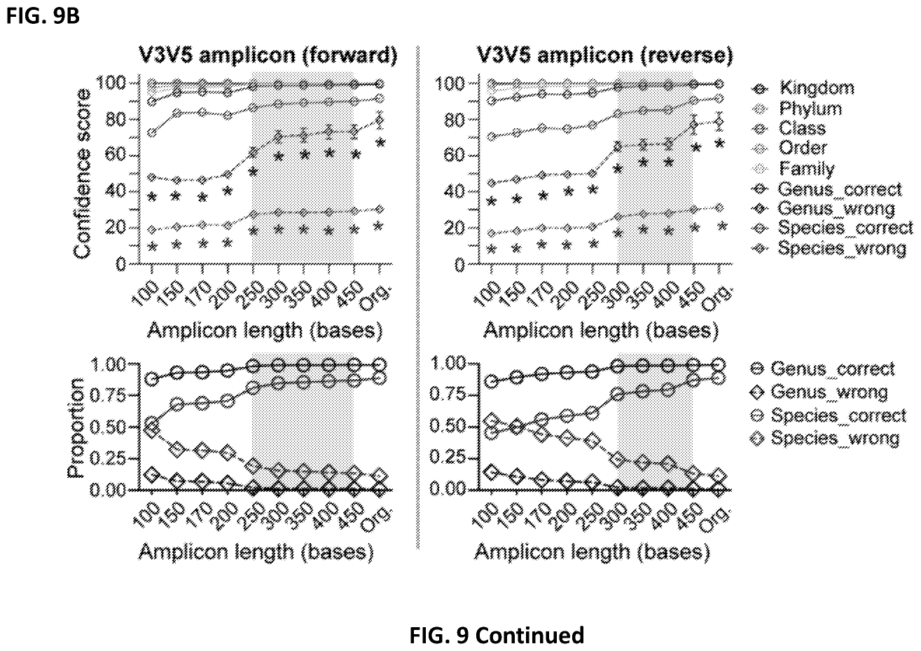

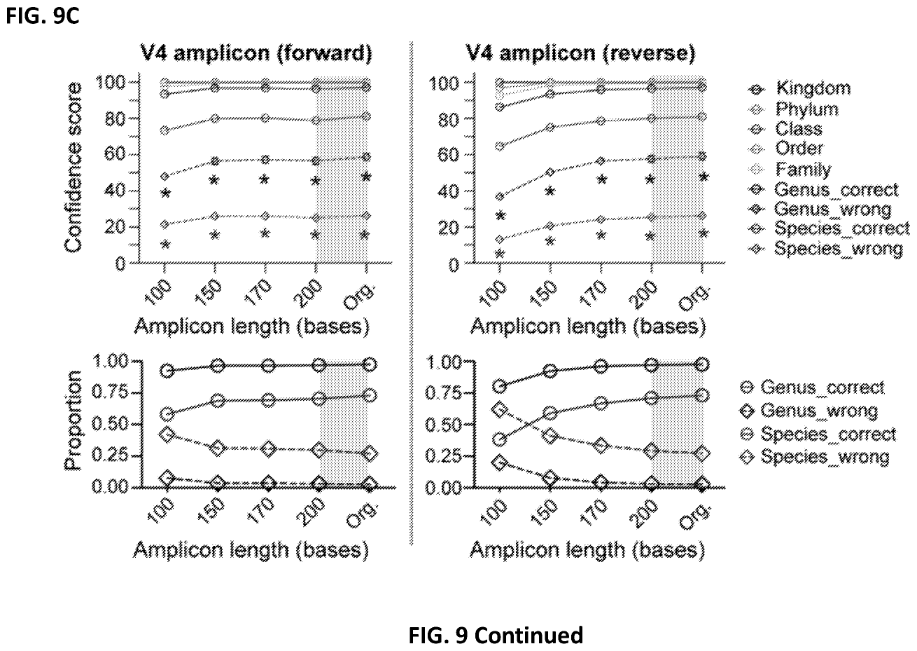

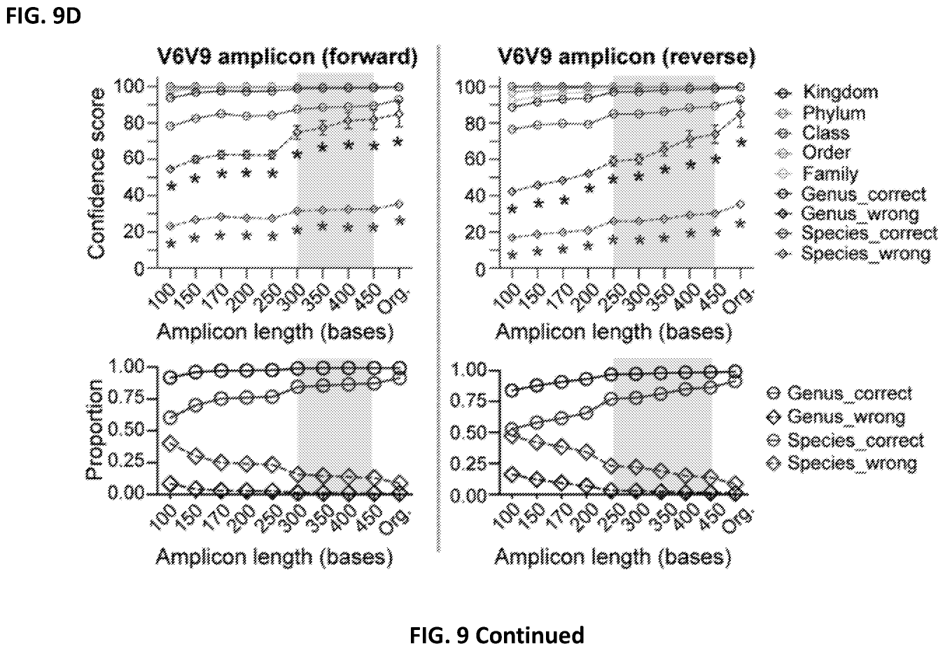

In some embodiments, a step in determining features suitable for classification of microbiome dysbiosis associated disease state and/or determination of appropriate treatment methods comprises benchmarking of taxonomic accuracy using simulated amplicons of variable length. In some embodiments, benchmarking taxonomic accuracy of BLCA, simulated amplicons of variable length are generated by trimming full-length amplicons derived from a suitable database (e.g., NCBI 16S RefSeq) from either forward or reverse orientation. In some embodiments, trimming of full-length amplicons results in 80, 100, 120, 140, 160, 180, 200, 220, 240, 260, 280, 300, 320, 340, 360, 380, 400, 420, 440, and/or 460 bases. In some embodiments, trimming of full-length amplicons results in 100, 150, 170, 200, 250, 300, 350, 400 and 450 bases for V1-V3, V3-V5 and V6-V9 amplicon data, and 100, 150, 170, 200 and 250 bases for V4 amplicon data. In some embodiments, suitable 16S amplicon lengths are determined according to FIG. 1B, FIGS. 7A-7C, FIG. 8, and/or FIGS. 9A-9D. In some embodiments, in addition to the known taxonomic lineage, the parent 16S sequences of simulated amplicons are also present in the BLCA default reference database using NCBI 16S RefSeq, thus taxonomic misclassification can be evaluated. In some embodiments, misclassification rate is defined as the proportion of incorrect annotations for simulated amplicons. In some embodiments, to further determine the optimal confidence threshold of BLCA for mitigating misclassification, amplicons with a selected sequence length range are combined to calculate the proportion of correct versus incorrect annotations using defined thresholds. In some embodiments, the already known taxonomic lineage, true positive (TP) and false negative (FN) hits are correct annotations, whereas true negative (TN) and false positive (FP) hits are incorrect annotations.

In most embodiments, a step in determining features suitable for classification of microbiome dysbiosis associated disease state and/or determination of appropriate treatment methods comprises design of a data analysis pipeline. In certain embodiments, the data analysis pipeline is the Taxa4Meta pipeline. In certain embodiments, data analysis pipelines are generated as a function of benchmarking results. In certain embodiments, a new computational pipeline “Taxa4Meta” can be used to analyze 16S amplicon data with an optimal range of variable sequence lengths. In some embodiments, such a pipeline implements several open-source programs, such as VSEARCH³¹ for stringent clustering with a known identity range (e.g., 90%, 91%, 92%, 93%, 94%, 95%, 96%, 97%, 98%, 99%, 100% identity; preferably 99% identity). In some embodiments, open-source programs such as VSEARCH can be optimized for 16S amplicon data with the selected variable lengths after quality trimming. In some embodiments, BLCA¹¹ can be used with optimal region-specific confidence thresholds for stringent species annotation of OTUs. In certain embodiments, IDTAXA¹⁰ can be utilized for annotating OTUs that cannot be annotated down to species resolution. In certain embodiments, collapsed taxonomic profiles from OTU tables are used for downstream analyses during 16S meta-analysis.

In some embodiments, a step in determining features suitable for classification of microbiome dysbiosis associated disease state and/or determination of appropriate treatment methods comprises benchmarking of taxonomic profiling accuracy comparing new data analysis pipelines (e.g., Taxa4Meta) with other standard 16S data analysis pipelines. In some embodiments, the feasibility and/or accuracy of different 16S pipelines are tested using the simulated and experimental datasets^{12,21}, and optionally the tests are designed to retain reads for accurate sequence clustering and for improved taxonomic accuracy. In some embodiments, simulated datasets are prepared from a suitable data base (e.g., NCBI 16S RefSeq as indicated above). In some embodiments, full-length amplicons of V1-V3, V3-V5, V4 and V6-V9 are simulated, and random sequence count ranging from 1 to 50 and random phred quality score (ASCII_BASE=33) ranging from 30 to 42 are generated for each full-length amplicon. In some embodiments, further length trimming is performed for one or more or each of the full-length amplicons, for example but not limited to, V1-V3 forward amplicons (200, 250, 300, 350, 400 and 450 bases), V1-V3 reverse amplicons (300, 350, 400 and 450 bases), V3-V5 forward amplicons (250, 300, 350, 400 and 450 bases), V3-V5 reverse amplicons (300, 350, 400 and 450 bases), both forward and reverse amplicons of V4 (200 and 250 bases), V6-V9 forward amplicons (300, 350, 400 and 450 bases), V6-V9 reverse amplicons (250, 300, 350, 400 and 450 bases). In some embodiments, trimmed amplicons from the same sequence orientation of the same 16S variable region are combined for benchmarking different 16S pipelines. In some embodiments, NCBI 16S taxonomic lineage of NCBI 16S RefSeq is used as the ground truth (reference annotations) for comparison. In some embodiments, a cohort (e.g., a Korean stool microbiome datasct¹²) with the same DNA extracts used for 454 V1-V4, Illumina V1-V3, Illumina V3-V4, Illumina V4, and Illumina shotgun metagenomic sequencing is used as the real human microbiome dataset for benchmarking different 16S pipelines. In some embodiments, primers retained in the sequence reads are removed by positional trimming. In some embodiments, Illumina paired-end reads are merged (e.g., using USEARCH (version 8.1.1831)) with certain parameters (e.g., default parameters) prior to benchmarking 16S pipelines. In certain embodiments, key 16S analysis pipelines can include DADA2-IDTAXA, DADA2-RDP, UCLUST-UCLUST, USEARCH-RDP, Taxa4Meta, Kraken2 and/or MetaPhlAn2. In some embodiments, key 16S analysis pipelines DADA2-IDTAXA, DADA2-RDP. UCLUST-UCLUST, USEARCH-RDP, Taxa4Meta, Kraken2 and/or MetaPhlAn2 are benchmarked with simulated amplicons and/or ground truth datasets (e.g., Korean human microbiome dataset).

In some embodiments, an analysis procedure for a DADA2-IDTAXA pipeline can be performed. In some embodiments, DADA2 (version 1.8) is used for denoising amplicon data after quality filtering with a maximum expected error (e.g., of 2) and a minimum base length (e.g., of 200 bases). In some embodiments, IDTAXA together with its pre-built RDP training set (version 16) is used for taxonomic annotation with the confidence threshold (e.g., of 70) using a number of bootstraps (e.g., 100 bootstraps). In some embodiments, IDTAXA based analysis can only go down to genus level. In some embodiments, an analysis procedure for DADA2-RDP pipeline can be performed. In some embodiments, DADA2 (version 1.8) is used for denoising amplicon data after quality filtering with a maximum expected error (e.g., of 2) and minimum base length (e.g., of 200 bases). In some embodiments, RDP Naive Bayesian Classifier algorithm implemented in DADA2's assignTaxonomy function together with its pre-formatted RDP training set (version 16) is used for taxonomic annotation using a minimum bootstrap confidence (e.g., a minimum bootstrap confidence of 50). In some embodiments, a DADA2-RDP analysis can go down to species level. In some embodiments, an analysis procedure for a UCLUST-UCLUST pipeline can be performed. In some embodiments. UCLUST (version 1.2.22q) is used for clustering amplicon data with known sequence similarity (e.g., of 97%) after quality filtering with the minimum quality threshold (e.g., of 20) and a minimum base length (e.g., of 140 bases). In some embodiments, representative sequence(s) of OTUs are selected (e.g., with pick_rep_set.py script) with default parameters. In some embodiments. UCLUST implemented in assign_taxonomy.py script together with SILVA database (release 123; choice of silva_132_97_16S.fna) is used for taxonomic annotation, which can be down to species level using a minimum bootstrap confidence (e.g., of 0.5). In some embodiments, one or more or all procedures are completed in the QIIME platform (version 1.9.1). In some embodiments, such a pipeline is similar to the meta-analysis method used by Mancabelli et al.²². In some embodiments, an analysis procedure for USEARCH-RDP pipeline can be performed. In some embodiments. USEARCH is used for clustering amplicon data with known sequence similarity (e.g., 100% sequence similarity) after quality filtering with a maximum expected error (e.g., of 2) and a minimum base length (e.g., of 200 bases). In some embodiments, RDP classifier (version 2.12) together with RDP training set (version 16) is used for taxonomic annotation, which can be down to species level using a minimum bootstrap confidence (e.g., of 0.5). In some embodiments, such a pipeline is similar to the meta-analysis method used by Duvallet et al.²⁰). In some embodiments, an analysis procedure for the Taxa4meta pipeline can be performed. In some embodiments. Taxa4Meta (e.g., version 1.22) is used for clustering amplicon data after quality filtering with a maximum expected error (e.g., of 2) and a selected range of variable lengths, optionally as suggested by Taxa4Meta itself. In some embodiments, taxonomic annotation by Taxa4Meta binary classifier can be down to species level. In some embodiments, an analysis procedure for Metagenomic classifiers can be performed. In some embodiments, Paired-end sequences are trimmed and filtered to meet a maximum expected error (e.g., of 2) with a minimum read length (e.g., of 50). In some embodiments, Kraken2 (version 2.0.8) with its pre-built database (minikraken2_v2_8 GB_201904_UPDATE) with default parameters is used for taxonomic profiling for shotgun metagenomic data. In some embodiments, MetaPhlAn2 (version 2.7.7) with it default database (mpa_v20_m200) with default parameters is used for taxonomic profiling for shotgun metagenomic data. In some embodiments, Kraken2 family-level abundance results are used as the reference for comparisons across different 16S pipelines. In some embodiments, given the high precision on species identification, MetaPhlAn2 species-level abundance results are used as the reference for evaluating species calls of different 16S pipelines. In some embodiments, a pseudo sample is created by averaging each family-level abundance of all WGS samples (e.g., 27 WGS samples), then the abundance-weighted Jaccard distance is calculated between the pseudo sample and any real sample analyzed by different pipelines.

In some embodiments, a step in determining features suitable for classification of microbiome dysbiosis associated disease state and/or determination of appropriate treatment methods comprises microbiome meta-analysis of diarrheal microbiome datasets. In some embodiments, one or more diarrheal datasets are run on the Taxa4Meta pipeline adopted optimal taxonomic thresholds for each 16S variable region. In some embodiments, Taxa4Meta command queries for diarrheal dataset are similar or the same as those indicated in Table 21. In some embodiments, relative abundance of collapsed species profiles generated from Taxa4Meta OTU count tables are used with or without rarefaction. In some embodiments, relative abundance of collapsed species profiles generated from Taxa4Meta OTU count tables require a minimum number of reads per sample. In some embodiments, a minimum number of reads per sample is 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500 or more or any range derivable therein. In some embodiments, a minimum number of reads per sample is 1,000 reads per sample. In some embodiments, if species is assigned by Taxa4Meta-BLCA, the taxonomic lineage from NCBI 16S RefSeq is adopted for that species to avoid inconsistency in taxonomic lineage. In some embodiments, merging of Taxa4Meta collapsed species of is based on taxonomic lineages.

In some embodiments, a step in determining features suitable for classification of microbiome dysbiosis associated disease state and/or determination of appropriate treatment methods comprises determining predictive metagenome functions. In some embodiments, predictive metagenome functions can be determined using open source software, e.g., PICRUSt2 (see e.g., Holmes et al., Generative models for microbial metagenomics. PLOS One 7, (2012)). In some embodiments, default Taxa4Meta parameters, OTU count tables, and/or OTU sequences are used to infer metabolic pathway abundance profiles for one or more datasets. In some embodiments, merging of PICRUSt2 pathway profiles is based on MetaCyc pathway IDs. In some embodiments, either or both LEfSe analysis (one-against-one test mode; version 1.0) and random forest (RF)-based feature ranking (default parameters in Orange version 3.20) are performed using pathway abundance profiles for diseased (e.g., CDI, IBD CD, IBD UC, and/or IBS) and/or control subjects. In some embodiments, mean decrease accuracy (MDA) score from RF-based analysis is used to rank pathways. In some embodiments, the top 20 pathways must be listed by both RF-based feature ranking result and LEfSe analysis result. In some embodiments, the top 2, 5, 10, 15, 20, 25, 30, 35, 40, 45, 50, 55, 60, 65, 70, 75, 80, 85, or 90 pathways are listed by both RF-based feature ranking results and LEFSe analysis results. In some embodiments, the top ranked pathways are selected for subsequent analysis. In some embodiments, the top ranked pathways are the top 2, 5, 10, 15, 20, 25, 30, 35, 40, 45, 50, 55, 60, 65, 70, 75, 80, 85, or 90 pathways or any range derivable therein. In some embodiments, the top ranked pathways are features indicative of a disease state and/or suitable for binary classification of disease state (see e.g., Tables 1-8).

In some embodiments, data (e.g., relative abundances, associations, metabolic pathways, etc.) associated with a collection of one or more OTUs is collaposed into an enterotype. In some embodiments, an enterotype encompasses two or more OTUs. In some embodiments, an OTU may be collapsed into a simplified genera designation. In some embodiments, an OTU is not collapsed into a simplified genera designation.

In some embodiments, a step in determining features suitable for classification of microbiome dysbiosis associated disease state and/or determination of appropriate treatment methods comprises determining α-Diversity and/or β-diversity. In some embodiments, one or more α-diversity indices are calculated at OTU levels. In some embodiments, α-diversity indices are the Shannon index (e.g., alpha_diversity.py in QIIME v1.9.1) and/or the richness index (e.g., breakaway package version 4.7.5). In some embodiments, QIIME v1.9.1, principal coordinate analysis (PCoA) with abundance-weighted Jaccard distance metric is applied for β-diversity analysis using combined collapsed species profile. In some embodiments, ANOSIM test for group comparison is performed using the beta-diversity distance profile and the permutations of 999.

In some embodiments, a step in determining features suitable for classification of microbiome dysbiosis associated disease state and/or determination of appropriate treatment methods comprises fitting factors onto β-diversity ordination plot. In some embodiments, fitting factors (e.g., taxa) onto a two-dimensional ordination plot (e.g., first two coordinates) is performed using the envfit function in vegan package (version 2.5-7) or a suitable alternative program. In some embodiments, taxonomic abundance profile at family level is used as one of or the only factor in this analysis. In some embodiments, significance of fitted factors is established using the permutation of 999 in the envfit run.

In some embodiments, a step in determining features suitable for classification of microbiome dysbiosis associated disease state and/or determination of appropriate treatment methods comprises microbiome enterotyping. In some embodiments, microbiome enterotyping is performed with family abundance profiles of one or more or all meta-analysis training sets. In some embodiments, Dirichlet multinomial mixtures (DMM) algorithm, a classical method for clustering community profile data, is used for microbiome enterotyping.

In some embodiments, a step in determining features suitable for classification of microbiome dysbiosis associated disease state and/or determination of appropriate treatment methods comprises supervised classification and/or independent cohort validation. In some embodiments, supervised classification procedures are performed using Orange software³³ (e.g., version 3.20) or a suitable alternative thereof, and applied to the reported cohorts with clinical definitions. In some embodiments, an original sample grouping information from each cohort is adopted. In some embodiments, such an adoption is done so the gold standard definition is clear for each sample. In some embodiments, random forest-based feature ranking was used as a first pass to select the top 100 input features (e.g., taxa, or biochemical pathways) for downstream supervised learning. In some embodiments, unless performing sub-sampling, input samples are used for training procedure. In some embodiments, supervised classification is performed using individual learning algorithms including but not limited to Random Forest (RF), Support Vector Machine (SVM), Naïve Bayes (NB), and/or Neural Network (NN). In some embodiments, a Stack model as an aggregated meta-learner of RF, SVM and NB is assessed. In some embodiments, a 5-fold cross-validation method is applied for sub-sampling of training and test data during a training procedure. In some embodiments, receiver-operating-characteristic (ROC) analysis is performed using the training results. In some embodiments, values of area-under-the curve (AUC) and classification accuracy (CA) are calculated to evaluate the performance of each classification model. In some embodiments, a suitable AUC value is more than 0.80, 0.81, 0.82, 0.83, 0.84. 0.85, 0.86, 0.87, 0.88, 0.89, 0.90, 0.91, 0.92, 0.93, 0.94, 0.95, 0.96, 0.97, 0.98, or 0.99 or any range derivable therein. In some embodiments, a preferred AUC value is more than 0.95, 0.96, 0.97, 0.98, or 0.99 or any range derivable therein. In some embodiments, CA refers to the proportion of correct predicted samples from the classification model compared to the original clinical diagnosis. In some embodiments, a suitable CA value is more than 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, or 0.99 or any range derivable therein. In some embodiments, a preferred CA value is more than 0.90, 0.91, 0.92, 0.93, 0.94, 0.95, 0.96, 0.97, 0.98, or 0.99 or any range derivable therein. In some embodiments, independent validation of classification models is performed using datasets of recently reported microbiome surveys of human diarrheal diseases that were not included in the training set. In some embodiments, one or more validation datasets are analyzed individually using the Taxa4Meta pipeline to generate taxonomic profile data for validating classification models. In some embodiments, CDI and IBD scores refer to the predicted scores of each sample as the class of CDI and IBD, respectively.

In some embodiments, a step in determining features suitable for classification of microbiome dysbiosis associated disease state and/or determination of appropriate treatment methods comprises statistical analysis. In some embodiments, comparisons between two groups are made using non-parametric Mann-Whitney-Wilcoxon two-tailed test or a suitable alternative thereof, and comparisons for more than two groups are made using non-parametric Kruskal-Wallis two-tailed test or a suitable alternative thereof. In some embodiments, multiple comparisons and pairwise Spearman or Pearson correlations are adjusted using the Benjamini-Hochberg (BH) false discovery rate (p<0.05, regarded as statistically significant), or a suitable alternative thereof.

In some embodiments, calculation of a meta-feature value is performed by: (i) determining the feature value of at least two, preferably more features, (ii) “normalizing” the feature value of each individual feature by dividing the value with a coefficient which is approximately the median value of the respective feature in a representative cohort, and (iii) calculating the median of the group of normalized gene expression values. In some embodiments, meta-feature analysis is performed as described herein.

As disclosed herein, in some embodiments, a feature shall be understood to be specifically increased in presence if the abundance level of the feature is at least about 2-fold, 4-fold, 6-fold, 8-fold, 10-fold, 15-fold, 20-fold, 30-fold, 40-fold, 50-fold, 60-fold, 70-fold, 80-fold, 90-fold, 100-fold, 1000-fold, or 10000-fold higher (or any range derivable therein) than in a reference, or in a mixture of references. References include but are not limited to, biological samples from one or more otherwise healthy individuals, biological samples from one or more individuals diagnosed with a different disease, and/or non-diarrheal biological samples from one or more individuals. In some embodiments, references can include normalized values across a cohort.

In certain algorithms a suitable threshold level is first determined for a feature. The suitable threshold level can be determined from measurements of feature presence, absence, and/or levels (e.g., quantity, activity, etc.) in one or more individuals from a test cohort. In some embodiments, median feature values in a multiple expression measurement is taken as a suitable threshold value. In some embodiments, mean feature values in a multiple expression measurement is taken as a suitable threshold value. In some embodiments, mode feature values in a multiple expression measurement is taken as a suitable threshold value. Comparison of multiple features with a threshold level can be performed as follows: 1) The individual features are compared to their respective threshold levels, 2) The number of features, the level of which is above and/or below their respective threshold level, is determined, 3) If a feature value is above its respective threshold level, then the feature level of is taken to be “above the threshold level”, 4) If a feature value is below its respective threshold level, then the feature level is taken to be “below the threshold level”.

In some embodiments, a disease classification may be determined from analysis of a sufficiently large number of features. In this context, a sufficiently large number of features means preferably 10%, 15%, 20%, 25%, 30%, 40%, 50%, 60%, 70%, 75%, 80%, 85%, 90%, 95%, 98%, 99%, or 100% of the features described by one or more binary tests disclosed herein (see e.g., Tables 2-17).

In certain aspects, the determination of feature presence, absence, and/or levels is on a substrate that allows evaluation of RNA molecule levels from a given sample, such as a gene chip, for example but not limited to Affymetrix™ gene chip, NanoString nCounter™, Illlumina BeadChip™, etc.,

In some embodiments, the determination of feature presence, absence, and/or levels is by 16S rRNA sequencing.

In some embodiments, the determination of feature presence, absence, and/or levels is by RNA sequencing.

In some embodiments, the determination of feature presence, absence, and/or levels is by whole genome sequencing, for example but not limited to, whole genome shotgun sequencing.

In other embodiments, the determination of feature presence, absence, and/or levels is done by polymerase chain reaction (PCR), for example but not limited to, real-time PCR, quantitative real time PCR, reverse transcriptase PCR, multiplexed PCR, nested PCR, long-range PCR, single-cell PCR, fast-cycling PCR, methylation-specific PCR, hot start PCR, high-fidelity PCR, in situ PCR, etc.

In some embodiments, the determination of feature presence, absence, and/or levels is performed by measuring proteins, polypeptides, metabolites, small molecules, etc. instead of nucleic based analyses (e.g., RNA and/or DNA based analyses). In some embodiments, techniques suitable for measuring the same include but are not limited to methods such as western blotting, IP-MS/MS, LC-MS/MS, NMR, PQN, ELISAs, HPLC, etc.

### B. Nucleic Acid Based Assays

Screening methods based on differential levels of genes and/or gene products are common place in the art. In accordance with one aspect of the present invention, the differential patterns of features can be determined by measuring the levels of RNA transcripts indicative of these features, or genes whose expression is modulated by the presence or absence of one or more of these features, present in a patient's biological sample (e.g., a fecal sample, swab, irrigation, mucosal biopsy, etc.). Suitable methods for this purpose include, but are not limited to, DNA sequencing, RNA sequencing, RT-PCR, Northern Blot, in situ hybridization, Southern Blot, slot-blotting, nuclease protection assay, and oligonucleotide arrays.

In some embodiments, feature absence, presence, and/or levels are determined from a biological sample obtained from a fecal sample, intestinal biopsy, intestinal swab, mucosal biopsy, and/or intestinal irrigation. In certain embodiments, feature absence, presence, and/or levels are preferably determined from a biological sample obtained from a fecal sample. In some embodiments, a biological sample may be a fixated samples (e.g., those fixed using formalin, paraformaldehyde, paraffin, etc.), blood, tears, semen, saliva, urine, feces, tissue, breast milk, lymph fluid, stool, sputum, cerebrospinal fluid, and/or supernatant from cell lysate.

In certain aspects, RNA isolated from a biological sample can be amplified to cDNA or cRNA before detection and/or quantitation. In some embodiments, isolated RNA can be either total RNA or mRNA. In some embodiments, RNA amplification can be specific or non-specific. In some embodiments, suitable amplification methods include, but are not limited to, reverse transcriptase PCR, isothermal amplification, ligase chain reaction, and Qbeta replicase. In some embodiments, amplified nucleic acid products can be detected and/or quantitated through hybridization to labeled probes. In some embodiments, detection may involve fluorescence resonance energy transfer (FRET) or some other kind of quantum dots.

In some embodiments, amplification primers or hybridization probes for detection of presence, absence, and/or levels of a feature can be prepared from a gene sequence or obtained through commercial sources, such as Affymetrix, NanoString, Illumina BeadChip, etc. In certain embodiments a gene sequence is identical or complementary to at least 8, 10, 12, 14, 16, 18, or 20 contiguous nucleotides of a coding sequence.

In some embodiments, sequences suitable for making probes/primers for detection of a corresponding feature includes those that are identical or complementary to all or part of one or more genes specific to taxonomic units described herein. In some embodiments, sequences suitable for making probes/primers for detection of a corresponding feature includes those that are unique to one or more genes specific to taxonomic units described herein.

In some embodiments, use of a probe or primer of between 13 and 100 nucleotides, preferably between 17 and 100 nucleotides in length, or in some aspects of the invention up to 1-2 kilobases or more in length, allows the formation of a duplex molecule that is both stable and selective. Molecules having complementary sequences over contiguous stretches greater than 20 bases in length are generally preferred, to increase stability and/or selectivity of the hybrid molecules obtained. One will generally prefer to design nucleic acid molecules for hybridization having one or more complementary sequences of 20 to 30 nucleotides, or even longer where desired. Such fragments may be readily prepared, for example, by directly synthesizing the fragment by chemical means or by introducing selected sequences into recombinant vectors for recombinant production.

In some embodiments, each probe/primer comprises at least 15 nucleotides. For instance, each probe can comprise at least or at most 20, 25, 50, 75, 100, 125, 150, 175, 200, 225, 250, 275, 300, 325, 350, 400 or more nucleotides (or any range derivable therein). They may have these lengths and have a sequence that is identical or complementary to a gene or portion of a genome of a taxonomic unit described herein. Preferably, each probe/primer has relatively high sequence complexity and does not have any ambiguous residue (undetermined “n” residues). In some embodiments, probes/primers can hybridize to a target gene, including its RNA transcripts, under stringent or highly stringent conditions. In some embodiments, because each of the features has more gene sequences, it is contemplated that probes and primers may be designed for use with any one or more of these gene sequences. For example, inosine is a nucleotide frequently used in probes or primers to hybridize to more than one sequence. It is contemplated that probes or primers may have inosine or other design implementations that accommodate recognition of more than one sequence for a particular feature.

For applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting specific transcripts. It is generally appreciated that conditions can be rendered more stringent by the addition of increasing amounts of formamide.

In some embodiments, probes/primers for a gene are selected from regions which significantly diverge from the sequences of other genes. Such regions can be determined by checking the probe/primer sequences against relevant genome sequence databases. One algorithm suitable for this purpose is the BLAST algorithm. This algorithm involves first identifying high scoring sequence pairs (HSPs) by identifying short words of length (W) in the query sequence, which either match or satisfy some positive-valued threshold score (T) when aligned with a word of the same length in a database sequence. T is referred to as the neighborhood word score threshold. These initial neighborhood word hits act as seeds for initiating searches to find longer HSPs containing them. The word hits are then extended in both directions along each sequence to increase the cumulative alignment score. Cumulative scores are calculated using, for nucleotide sequences, the parameters M (reward score for a pair of matching residues; always >0) and N (penalty score for mismatching residues; always <0). The BLAST algorithm parameters W. T. and X determine the sensitivity and speed of the alignment. These parameters can be adjusted for different purposes, as appreciated by one of ordinary skill in the art.

In some embodiments, quantitative RT-PCR (such as TaqMan, ABI) is used for detecting and comparing the levels of RNA transcripts in biological samples. Quantitative RT-PCR involves reverse transcription (RT) of RNA to cDNA followed by relative quantitative PCR (RT-PCR). The concentration of the target DNA in the linear portion of the PCR process is proportional to the starting concentration of the target before the PCR was begun. By determining the concentration of the PCR products of the target DNA in PCR reactions that have completed the same number of cycles and are in their linear ranges, it is possible to determine the relative concentrations of the specific target sequence in the original DNA mixture. If the DNA mixtures are cDNAs synthesized from RNAs isolated from different tissues or cells, the relative abundances of the specific transcripts from which the target sequence was derived may be determined for the respective cells. This direct proportionality between the concentration of the PCR products and the relative transcript abundances is true in the linear range portion of the PCR reaction. The final concentration of the target DNA in the plateau portion of the curve is determined by the availability of reagents in the reaction mix and is independent of the original concentration of target DNA. Therefore, the sampling and quantifying of the amplified PCR products preferably are carried out when the PCR reactions are in the linear portion of their curves. In addition, relative concentrations of the amplifiable cDNAs preferably are normalized to some independent standard, which may be based on either internally existing RNA species or externally introduced RNA species. The abundance of a particular transcript or DNA species may also be determined relative to the average abundance of all transcript or DNA species in the sample.

In some embodiments, PCR amplification utilizes one or more internal PCR standards. The internal standard may be an abundant housekeeping gene in a cell. These standards may be used to normalize expression and/or abundance levels so that the expression and/or abundance levels of different features can be compared directly. A person of ordinary skill in the art would know how to use an internal standard to normalize expression and/or abundance levels.

A problem inherent in clinical samples is that they are generally of variable quantity and/or quality. In some embodiments, this problem can be overcome if the RT-PCR is performed as a relative quantitative RT-PCR with an internal standard in which the internal standard is an amplifiable nucleic acid fragment that is similar or larger than the target nucleic acid fragment and in which the abundance of the nucleic acid fragment encoding the internal standard is roughly 5-100 fold higher than the nucleic acid fragment encoding the target. This assay measures relative abundance, not absolute abundance of the respective nucleic acid species.

In another embodiment, the relative quantitative RT-PCR uses an external standard protocol. Under this protocol, the PCR products are sampled in the linear portion of their amplification curves. The number of PCR cycles that are optimal for sampling can be empirically determined for each target nucleic acid fragment.

Nucleic acid arrays can also be used to detect and compare the differential presence, absence, or levels of microbiome dysbiosis features. Probes suitable for detecting the corresponding features can be stably attached to known discrete regions on a solid substrate. As used herein, a probe is “stably attached” to a discrete region if the probe maintains its position relative to the discrete region during the hybridization and the subsequent washes. Construction of nucleic acid arrays is well known in the art. Suitable substrates for making polynucleotide arrays include, but are not limited to, membranes, films, plastics and quartz wafers.

A nucleic acid array can comprise at least 1, 2, 3, 4, 5, 6, 7, 8, 9, 10, 15, 20, 25, 30, 35, 40, 45, 50, 60, 70, 80, 90, 100, 150, 200, 250 or more different polynucleotide probes, which may hybridize to different and/or the same targets representative of one or more features. Multiple probes for the same feature can be used on a single nucleic acid array. Probes for other features can also be included in the nucleic acid array. Probe combinations suitable for delincation of healthy, CDI, IBS, IBD UC, and/or IBD CD can be included on a nucleic acid array. The probe density on the array can be in any range. In some embodiments, the density may be 50, 100, 200, 300, 400, 500 or more probes/cm2.

Specifically contemplated by the present inventors are chip-based nucleic acid technologies such as those described by Hacia et al. (1996) and Shoemaker et al. (1996). Briefly, these techniques involve quantitative methods for analyzing large numbers of genes rapidly and accurately. By tagging genes with oligonucleotides or using fixed probe arrays, one can employ chip technology to segregate target molecules as high density arrays and screen these molecules on the basis of hybridization (see also, Pease et al., 1994; and Fodor et al, 1991). It is contemplated that this technology may be used in conjunction with evaluating the presence, absence, and/or levels of one or more features with respect to diagnostic, prognostic, and treatment methods of the disclosure.

The present disclosure may involve the use of arrays or data generated from an array. Data may be readily available. Moreover, an array may be prepared in order to generate data that may then be used in correlation studies.

An array generally refers to ordered macroarrays or microarrays of nucleic acid molecules (probes) that are fully or nearly complementary or identical to a plurality genes and/or gene products and that are positioned on a support material in a spatially separated organization. Macroarrays are typically sheets of nitrocellulose or nylon upon which probes have been spotted. Microarrays position the nucleic acid probes more densely such that up to 10,000 nucleic acid molecules can be fit into a region typically 1 to 4 square centimeters. Microarrays can be fabricated by spotting nucleic acid molecules, e.g., genes, oligonucleotides, etc., onto substrates or fabricating oligonucleotide sequences in situ on a substrate. Spotted or fabricated nucleic acid molecules can be applied in a high density matrix pattern of up to about 30 non-identical nucleic acid molecules per square centimeter or higher, e.g. up to about 100 or even 1000 per square centimeter. Microarrays typically use coated glass as the solid support, in contrast to the nitrocellulose-based material of filter arrays. By having an ordered array of complementing nucleic acid samples, the position of each sample can be tracked and linked to the original sample. A variety of different array devices in which a plurality of distinct nucleic acid probes are stably associated with the surface of a solid support are known to those of skill in the art. Useful substrates for arrays include nylon, glass and silicon. Such arrays may vary in a number of different ways, including average probe length, sequence or types of probes, nature of bond between the probe and the array surface, e.g. covalent or non-covalent, and the like. The labeling and screening methods of the present invention and the arrays are not limited in its utility with respect to any parameter except that the probes detect absence, presence, or levels of one or more features; consequently, methods and compositions may be used with a variety of different types of genes and/or gene products.

Representative methods and apparatus for preparing a microarray have been described, for example, in U.S. Pat. Nos. 5,143,854; 5,202,231; 5,242,974; 5,288,644; 5,324,633; 5,384,261; 5,405,783; 5,412,087; 5,424,186; 5,429,807; 5,432,049; 5,436,327; 5,445,934; 5,468,613; 5,470,710; 5,472,672; 5,492,806; 5,525,464; 5,503,980; 5,510,270; 5,525,464; 5,527,681; 5,529,756; 5,532,128; 5,545,531; 5,547,839; 5,554,501; 5,556,752; 5,561,071; 5,571,639; 5,580,726; 5,580,732; 5,593,839; 5,599,695; 5,599,672; 5,610,287; 5,624,711; 5,631,134; 5,639,603; 5,654,413; 5,658,734; 5,661,028; 5,665,547; 5,667,972; 5,695,940; 5,700,637; 5,744,305; 5,800,992; 5,807,522; 5,830,645; 5,837,196; 5,871,928; 5,847,219; 5,876,932; 5,919,626; 6,004,755; 6,087,102; 6,368,799; 6,383,749; 6,617,112; 6,638,717; 6,720,138, as well as WO 93/17126; WO 95/11995; WO 95/21265; WO 95/21944; WO 95/35505; WO 96/31622; WO 97/10365; WO 97/27317; WO 99/35505; WO 09923256; WO 09936760; WO 0138580; WO 0168255; WO 03020898; WO 03040410; WO 03053586; WO 03087297; WO 03091426; WO 03100012; WO 04020085; WO 04027093; EP 373 203; EP 785 280; EP 799 897 and UK 8 803 000; the disclosures of which are all herein incorporated by reference.

It is contemplated that the arrays can be high density arrays, such that they contain 100 or more different probes. It is contemplated that they may contain 1000, 16,000, 65,000, 250,000 or 1,000,000 or more different probes. The probes can be directed to targets in one or more different organisms. The oligonucleotide probes range from 5 to 50, 5 to 45, 10 to 40, or 15 to 40 nucleotides in length in some embodiments. In certain embodiments, the oligonucleotide probes are 20 to 25 nucleotides in length.

The location and sequence of each different probe sequence in the array are generally known. Moreover, the large number of different probes can occupy a relatively small area providing a high density array having a probe density of generally greater than about 60, 100, 600, 1000, 5,000, 10,000, 40,000, 100,000, or 400,000 different oligonucleotide probes per cm2. The surface area of the array can be about or less than about 1, 1.6, 2, 3, 4, 5, 6, 7, 8, 9, or 10 cm2.

Moreover, a person of ordinary skill in the art could readily analyze data generated using an array. Such protocols include information found in WO 9743450; WO 03023058; WO 03022421; WO 03029485; WO 03067217; WO 03066906; WO 03076928; WO 03093810; WO 03100448, all of which are specifically incorporated by reference.

In some embodiments, nuclease protection assays are used to quantify RNAs derived from a biological sample. There are many different versions of nuclease protection assays known to those practiced in the art. The common characteristic that these nuclease protection assays have is that they involve hybridization of an antisense nucleic acid with the RNA to be quantified. The resulting hybrid double-stranded molecule is then digested with a nuclease that digests single-stranded nucleic acids more efficiently than double-stranded molecules. The amount of antisense nucleic acid that survives digestion is a measure of the amount of the target RNA species to be quantified. An example of a nuclease protection assay that is commercially available is the RNase protection assay manufactured by Ambion, Inc. (Austin, Tex.).

In some embodiments, the presence, absence, and/or levels of one or more features are determined from a biological sample using 3′ RNA sequencing, using products such as Lexogen QuantSeq, QioSeq UPX 3′ Transcriptome, etc. In some embodiments, 3′ RNA sequencing does not require transcripts to be fragmented before reverse transcription, and cDNAs are reverse transcribed only from the 3′ RNA sequencing end of the transcripts, resulting in only one copy of cDNA for each transcript, resulting in a direct 1:1 ratio between RNA and cDNA copy numbers.

In some embodiments, gene expression is determined from a biological sample using specific targeted sequencing, using products such as BioSpyder Temp0-Seq. Ion Ampliseq Transcriptome, etc. In some embodiments, specific targeted sequencing targets RNA sequences by hybridization to DNA oligos followed by removal of unhybridized oligos and amplification of remaining products.

C. Proteins, Polypeptide, Metabolites, Etc. Based Assays

In other embodiments, the differential features (e.g., taxonomic and/or metabolic pathway biomarkers) can be determined by measuring levels of polypeptides encoded by components of the microbiome in a biological sample (e.g., a fecal sample, intestinal swap, intestinal biopsy, intestinal irrigation sample, etc.). Methods suitable for this purpose include, but are not limited to, immunoassays such as ELISA, RIA, FACS, dot blot, Western Blot, immunohistochemistry, and antibody-based radioimaging. Protocols for carrying out these immunoassays are well known in the art. Other methods such as 2-dimensional SDS-polyacrylamide gel electrophoresis can also be used. These procedures may be used to recognize any of the polypeptides encoded or implicated by one or more features described herein.

One example of a method suitable for detecting the levels of target proteins in biological samples is ELISA. In an exemplifying ELISA, antibodies capable of binding to the target proteins encoded by the genome of one or more features are immobilized onto a selected surface exhibiting protein affinity, such as wells in a polystyrene or polyvinylchloride microtiter plate. Then, samples to be tested are added to the wells. After binding and washing to remove non-specifically bound immunocomplexes, the bound antigen(s) can be detected. Detection can be achieved by the addition of a second antibody which is specific for the target proteins and is linked to a detectable label. Detection may also be achieved by the addition of a second antibody, followed by the addition of a third antibody that has binding affinity for the second antibody, with the third antibody being linked to a detectable label. Proper extraction procedures can be used to separate the target proteins from potentially interfering substances.

In another ELISA embodiment, one or more samples containing the target proteins reflective of one or more features are immobilized onto the well surface and then contacted with antibodies. After binding and washing to remove non-specifically bound immunocomplexes, the bound antigen is detected. Where the initial antibodies are linked to a detectable label, the immunocomplexes can be detected directly. The immunocomplexes can also be detected using a second antibody that has binding affinity for the first antibody, with the second antibody being linked to a detectable label.

Another typical ELISA involves the use of antibody competition in the detection. In this ELISA, the target proteins are immobilized on the well surface. The labeled antibodies are added to the well, allowed to bind to the target proteins, and detected by means of their labels. The amount of the target proteins in an unknown sample is then determined by mixing the sample with the labeled antibodies before or during incubation with coated wells. The presence of the target proteins in the unknown sample acts to reduce the amount of antibody available for binding to the well and thus reduces the ultimate signal.

Different ELISA formats can have certain features in common, such as coating, incubating or binding, washing to remove non-specifically bound species, and detecting the bound immunocomplexes. For instance, in coating a plate with either antigen or antibody, the wells of the plate can be incubated with a solution of the antigen or antibody, either overnight or for a specified period of hours. The wells of the plate are then washed to remove incompletely adsorbed material. Any remaining available surfaces of the wells are then “coated” with a non-specific protein that is antigenically neutral with regard to the test samples. Non-limiting examples of these nonspecific proteins include bovine serum albumin (BSA), casein and solutions of milk powder. The coating allows for blocking of nonspecific adsorption sites on the immobilizing surface and thus reduces the background caused by nonspecific binding of antisera onto the surface.

In ELISAs, a secondary or tertiary detection means can also be used. After binding of a protein or antibody to the well, coating with a non-reactive material to reduce background, and washing to remove unbound material, the immobilizing surface is contacted with the control and/or clinical or biological sample to be tested under conditions effective to allow immunocomplex (antigen/antibody) formation. These conditions may include, for example, diluting the antigens and antibodies with solutions such as BSA, bovine gamma globulin (BGG) and phosphate buffered saline (PBS)/Tween and incubating the antibodies and antigens at room temperature for about 1 to 4 hours or at 4° C. overnight. Detection of the immunocomplex then requires a labeled secondary binding ligand or antibody, or a secondary binding ligand or antibody in conjunction with a labeled tertiary antibody or third binding ligand.

After all of the incubation steps in an ELISA, the contacted surface can be washed so as to remove non-complexed material. For instance, the surface may be washed with a solution such as PBS/Tween, or borate buffer. Following the formation of specific immunocomplexes between the test sample and the originally bound material, and subsequent washing, the occurrence of the amount of immunocomplexes can be determined.

To provide a detecting means, the second or third antibody can have an associated label to allow detection. In some embodiments, a label is an enzyme that generates color development upon incubating with an appropriate chromogenic substrate. Thus, for example, one may contact and incubate the first or second immunocomplex with a urease, glucose oxidase, alkaline phosphatase or hydrogen peroxidase-conjugated antibody for a period of time and under conditions that favor the development of further immunocomplex formation (e.g., incubation for 2 hours at room temperature in a PBS-containing solution such as PBS-Tween).

After incubation with a labeled antibody, and subsequent to washing to remove unbound material, the amount of label is quantified, e.g., by incubation with a chromogenic substrate such as urea and bromocresol purple or 2,2′-azido-di-(3-ethyl)-benzhiazoline-6-sulfonic acid (ABTS) and hydrogen peroxide, in the case of peroxidase as the enzyme label. Quantitation can be achieved by measuring the degree of color generation, e.g., using a spectrophotometer.

In some embodiments, another suitable method is RIA (radioimmunoassay). An example of RIA is based on the competition between radiolabeled-polypeptides and unlabeled polypeptides for binding to a limited quantity of antibodies. Suitable radiolabels include, but are not limited to, I125. In some embodiments, a fixed concentration of I125-labeled polypeptide is incubated with a series of dilution of an antibody specific to the polypeptide. When the unlabeled polypeptide is added to the system, the amount of the I125-polypeptide that binds to the antibody is decreased. A standard curve can therefore be constructed to represent the amount of antibody-bound I125-polypeptide as a function of the concentration of the unlabeled polypeptide. From this standard curve, the concentration of the polypeptide in unknown samples can be determined. Various protocols for conducting RIA to measure the levels of polypeptides in a sample are well known in the art.

In some embodiments, suitable antibodies for biomarker detection include, but are not limited to, polyclonal antibodies, monoclonal antibodies, chimeric antibodies, humanized antibodies, single chain antibodies, Fab fragments, and fragments produced by a Fab expression library.

In some embodiments, antibodies can be labeled with one or more detectable moieties to allow for detection of antibody-antigen complexes. In some embodiments, detectable moieties can include compositions detectable by spectroscopic, enzymatic, photochemical, biochemical, bioelectronic, immunochemical, electrical, optical or chemical means. In some embodiments, detectable moieties include, but are not limited to, radioisotopes, chemiluminescent compounds, labeled binding proteins, heavy metal atoms, spectroscopic markers such as fluorescent markers and dyes, magnetic labels, linked enzymes, mass spectrometry tags, spin labels, electron transfer donors and acceptors, and the like.

Protein array technology is discussed in detail in Pandey and Mann (2000) and MacBeath and Schreiber (2000), each of which is herein specifically incorporated by reference. These arrays typically contain thousands of different proteins or antibodies spotted onto glass slides or immobilized in tiny wells and allow one to examine the biochemical activities and binding profiles of a large number of proteins at once. To examine protein interactions with such an array, a labeled protein is incubated with each of the target proteins immobilized on the slide, and then one determines which of the many proteins the labeled molecule binds. In certain embodiments such technology can be used to quantitate a number of proteins in a sample, such as a sample comprising a representative population of a microbiome.

The basic construction of protein chips has some similarities to DNA chips, such as the use of a glass or plastic surface dotted with an array of molecules. These molecules can be DNA or antibodies that are designed to capture proteins. Defined quantities of proteins are immobilized on each spot, while retaining some activity of the protein. With fluorescent markers or other methods of detection revealing the spots that have captured these proteins, protein microarrays are being used as powerful tools in high-throughput proteomics and drug discovery.

The earliest and best-known protein chip is the ProteinChip by Ciphergen Biosystems Inc. (Fremont, Calif.). The ProteinChip is based on the surface-enhanced laser desorption and ionization (SELDI) process. Known proteins are analyzed using functional assays that are on the chip. For example, chip surfaces can contain enzymes, receptor proteins, or antibodies that enable researchers to conduct protein-protein interaction studies, ligand binding studies, or immunoassays. With state-of-the-art ion optic and laser optic technologies, the ProteinChip system detects proteins ranging from small peptides of less than 1000 Da up to proteins of 300 kDa and calculates the mass based on time-of-flight (TOF).

The ProteinChip biomarker system is the first protein biochip-based system that enables biomarker pattern recognition analysis to be done. This system allows researchers to address important clinical questions by investigating the proteome from a range of crude clinical samples (i.e., laser capture microdissected cells, biopsies, tissue, urine, and serum). The system also utilizes biomarker pattern software that automates pattern recognition-based statistical analysis methods to correlate protein expression patterns from clinical samples with disease phenotypes.

In some embodiments, the levels of polypeptides in a biological sample can be determined by detecting the biological activities associated with the polypeptides. If a biological function/activity of a polypeptide is known, suitable in vitro bioassays can be designed to evaluate the biological function/activity, thereby determining the amount of the polypeptide in the sample.

In some embodiments, the levels of polypeptides and/or metabolites in a biological sample can be determined by IP-MS/MS and/or HPLC.

### D. Methods of Delineation Between Disease Classification States.

In certain embodiments, one or more features identified herein can be used to delineate between disease classification states, and/or to provide stake holders with a basis for prescribing one or more appropriate methods of treatment.

In certain embodiments, one or more features are the presence, absence, and/or level of one or more a metabolic pathways. In certain embodiments, one or more features associated with a metabolic pathway are described in any one of tables 1-8, and 18.

In certain embodiments, one or more features are the presence, absence, and/or level of one or more taxonomic unit. In certain embodiments, one or more features associated a taxonomic unit are described in any one of tables 9-17, and 19.

In certain embodiments, one or more features are the presence, absence, and/or level of one or more taxonomic units represented by: Bacteroides; Eubacterium rectale; Ruminococcus; Faecalibacterium; Enterococcus; Enterobacteriaceae; Roseburia; Coprococcus; Dorea; Lachnoclostridium; Clostridium XIVa; Erysipelatoclostridium; Alistipes; Fusicatenibacter; Odoribacter; Lactobacillus; Anaerostipes; Collinsella; Clostridioides; Klebsiella; Agathobaculum butyriciproducens; Veillonella; Phascolarctobacterium; Adlercreutzia; Clostridium; Eggerthella; Sutterellaceae Parasutterella; barnesiella; Eubacterium; Clostridium IV; Gemmiger; Streptococcus; Dialister; Escherichia; Colidextribacter; Oxalobacter; Prevotella; Clostridium XVIII; Actinomyces; and Fusobacterium.

In certain embodiments, one or more features associated with the presence, absence, and/or level of one or more a metabolic pathways is used in conjunction with one or more features associated with the presence, absence, and/or level of one or more taxonomic units. In certain embodiments, one or more features associated with a feature are described in any one of tables 1-19.

In certain embodiments, a disease classification can be determined by the presence, absence, or relative level of at least one of AST-PWY (L-arginine degradation II (AST pathway)), ECASYN-PWY (enterobacterial common antigen biosynthesis), THREOCAT-PWY (superpathway of L-threonine metabolism), PPGPPMET-PWY (ppGpp biosynthesis), PWY0-1338 (polymyxin resistance), PWY-6263 (superpathway of menaquinol-8 biosynthesis II), PWY-7371 (1,4-dihydroxy-6-naphthoate biosynthesis II), PWY-7374 (1,4-dihydroxy-6-naphthoate biosynthesis I), P221-PWY (octane oxidation), PWY-6749 (CMP-legionaminate biosynthesis I), PWY-7456 (mannan degradation), NONMEVIPP-PWY (methylerythritol phosphate pathway I), PWY-5097 (L-lysine biosynthesis VI), PWY-5505 (L-glutamate and L-glutamine biosynthesis), PWY-6122 (5-aminoimidazole ribonucleotide biosynthesis II), PWY-7663 (gondoate biosynthesis (anaerobic)), THRESYN-PWY (superpathway of L-threonine biosynthesis), HEMESYN2-PWY (heme biosynthesis II (anaerobic)), PWY-5304 (superpathway of sulfur oxidation (archaca), PWY-6478 (GDP-D-glycero-alpha-D-manno-heptose biosynthesis), PWY-7198 (pyrimidine deoxyribonucleotides de novo biosynthesis IV), and/or PWY-7210 (pyrimidine deoxyribonucleotides biosynthesis from CTP).

In certain embodiments, an increased abundance relative to an appropriate control of at least one of or all of AST-PWY (L-arginine degradation II (AST pathway)), ECASYN-PWY (enterobacterial common antigen biosynthesis), THREOCAT-PWY (superpathway of L-threonine metabolism), PPGPPMET-PWY (ppGpp biosynthesis), and/or PWY0-1338 (polymyxin resistance) is associated with CDI causative diarrhea. In certain embodiments, following detection of one or more of the indicative features, an individual is then treated accordingly.

In certain embodiments, an increased abundance relative to an appropriate control of at least one of or all of PWY-6263 (superpathway of menaquinol-8 biosynthesis II), PWY-7371 (1,4-dihydroxy-6-naphthoate biosynthesis II), PWY-7374 (1,4-dihydroxy-6-naphthoate biosynthesis I), P221-PWY (octane oxidation), PWY-6749 (CMP-legionaminate biosynthesis I), and/or PWY-7456 (mannan degradation) is associated with IBD UC causative diarrhea. In certain embodiments, following detection of one or more of the indicative features, an individual is then treated accordingly.

In certain embodiments, an increased abundance relative to an appropriate control of at least one of or all of NONMEVIPP-PWY (methylerythritol phosphate pathway I), PWY-5097 (L-lysine biosynthesis VI), PWY-5505 (L-glutamate and L-glutamine biosynthesis), PWY-6122 (5-aminoimidazole ribonucleotide biosynthesis II), PWY-7663 (gondoate biosynthesis (anaerobic)), THRESYN-PWY (superpathway of L-threonine biosynthesis), HEMESYN2-PWY (heme biosynthesis II (anaerobic)), PWY-5304 (superpathway of sulfur oxidation (archaca), PWY-6478 (GDP-D-glycero-alpha-D-manno-heptose biosynthesis), PWY-7198 (pyrimidine deoxyribonucleotides de novo biosynthesis IV), and/or PWY-7210 (pyrimidine deoxyribonucleotides biosynthesis from CTP) is associated with IBD causative. In certain embodiments, following detection of one or more of the indicative features, an individual is then treated accordingly.

In certain embodiments, methods described herein for classification of a disease state may comprise, or expressly does not comprise, detection and/or quantification of Acidaminococcaceae Acidaminococcus, or a metabolic pathway associated therewith.

In certain embodiments, methods described herein for classification of a disease state may comprise, or expressly does not comprise, detection and/or quantification of Acidaminococcaceae Phascolarctobacterium, or a metabolic pathway associated therewith.

In certain embodiments, methods described herein for classification of a disease state may comprise, or expressly does not comprise, detection and/or quantification of Actinobacteria Actinobacteria, or a metabolic pathway associated therewith.

In certain embodiments, methods described herein for classification of a disease state may comprise, or expressly does not comprise, detection and/or quantification of Actinomycetaceae Actinomyces, or a metabolic pathway associated therewith.

In certain embodiments, methods described herein for classification of a disease state may comprise, or expressly does not comprise, detection and/or quantification of Actinomycetaceae Schaalia, or a metabolic pathway associated therewith.

In certain embodiments, methods described herein for classification of a disease state may comprise, or expressly does not comprise, detection and/or quantification of Actinomycetales Actinomycetaceae, or a metabolic pathway associated therewith.

In certain embodiments, methods described herein for classification of a disease state may comprise, or expressly does not comprise, detection and/or quantification of Adlercreutzia equolifaciens, or a metabolic pathway associated therewith.

In certain embodiments, methods described herein for classification of a disease state may comprise, or expressly does not comprise, detection and/or quantification of Agathobaculum butyriciproducens, or a metabolic pathway associated therewith.

In certain embodiments, methods described herein for classification of a disease state may comprise, or expressly does not comprise, detection and/or quantification of Alistipes ihumii, or a metabolic pathway associated therewith.

In certain embodiments, methods described herein for classification of a disease state may comprise, or expressly does not comprise, detection and/or quantification of Alistipes obesi, or a metabolic pathway associated therewith.

In certain embodiments, methods described herein for classification of a disease state may comprise, or expressly does not comprise, detection and/or quantification of Alistipes shahii, or a metabolic pathway associated therewith.

In certain embodiments, methods described herein for classification of a disease state may comprise, or expressly does not comprise, detection and/or quantification of Anderostipes hadrus, or a metabolic pathway associated therewith.

In certain embodiments, methods described herein for classification of a disease state may comprise, or expressly does not comprise, detection and/or quantification of Atopobiaceae Atopobium, or a metabolic pathway associated therewith.

In certain embodiments, methods described herein for classification of a disease state may comprise, or expressly does not comprise, detection and/or quantification of Bacillales Gemella, or a metabolic pathway associated therewith.

In certain embodiments, methods described herein for classification of a disease state may comprise, or expressly does not comprise, detection and/or quantification of Bacillales Incertae Sedis Gemella, or a metabolic pathway associated therewith.

In certain embodiments, methods described herein for classification of a disease state may comprise, or expressly does not comprise, detection and/or quantification of Bacilli Lactobacillus, or a metabolic pathway associated therewith.

In certain embodiments, methods described herein for classification of a disease state may comprise, or expressly does not comprise, detection and/or quantification of Bacteria Proteobacteria, or a metabolic pathway associated therewith.

In certain embodiments, methods described herein for classification of a disease state may comprise, or expressly does not comprise, detection and/or quantification of Bacteroidales Rikenellaceae, or a metabolic pathway associated therewith.

In certain embodiments, methods described herein for classification of a disease state may comprise, or expressly does not comprise, detection and/or quantification of Bacteroides cellulosilyticus, or a metabolic pathway associated therewith.

In certain embodiments, methods described herein for classification of a disease state may comprise, or expressly does not comprise, detection and/or quantification of Bacteroides coprocola, or a metabolic pathway associated therewith.

In certain embodiments, methods described herein for classification of a disease state may comprise, or expressly does not comprise, detection and/or quantification of Bacteroides eggerthii, or a metabolic pathway associated therewith.

In certain embodiments, methods described herein for classification of a disease state may comprise, or expressly does not comprise, detection and/or quantification of Bacteroides koreensis, or a metabolic pathway associated therewith.

In certain embodiments, methods described herein for classification of a disease state may comprise, or expressly does not comprise, detection and/or quantification of Bacteroides nordii, or a metabolic pathway associated therewith.

In certain embodiments, methods described herein for classification of a disease state may comprise, or expressly does not comprise, detection and/or quantification of Bacteroides plebeius, or a metabolic pathway associated therewith.

In certain embodiments, methods described herein for classification of a disease state may comprise, or expressly does not comprise, detection and/or quantification of Bacteroides thetaiotaomicron, or a metabolic pathway associated therewith.

In certain embodiments, methods described herein for classification of a disease state may comprise, or expressly does not comprise, detection and/or quantification of Bacteroides xylanisolvens, or a metabolic pathway associated therewith.

In certain embodiments, methods described herein for classification of a disease state may comprise, or expressly does not comprise, detection and/or quantification of Bacteroidia Bacteroidales, or a metabolic pathway associated therewith.

In certain embodiments, methods described herein for classification of a disease state may comprise, or expressly does not comprise, detection and/or quantification of Betaproteobacteria, or a metabolic pathway associated therewith.

In certain embodiments, methods described herein for classification of a disease state may comprise, or expressly does not comprise, detection and/or quantification of Betaproteobacteria Burkholderiales, or a metabolic pathway associated therewith.

In certain embodiments, methods described herein for classification of a disease state may comprise, or expressly does not comprise, detection and/or quantification of Bifidobacteriales Bifidobacteriaceae, or a metabolic pathway associated therewith.

In certain embodiments, methods described herein for classification of a disease state may comprise, or expressly does not comprise, detection and/or quantification of Bifidobacterium adolescentis, or a metabolic pathway associated therewith.

In certain embodiments, methods described herein for classification of a disease state may comprise, or expressly does not comprise, detection and/or quantification of Bifidobacterium boum, or a metabolic pathway associated therewith.

In certain embodiments, methods described herein for classification of a disease state may comprise, or expressly does not comprise, detection and/or quantification of Blautia [Ruminococcus] gnavus, or a metabolic pathway associated therewith.

In certain embodiments, methods described herein for classification of a disease state may comprise, or expressly does not comprise, detection and/or quantification of Blautia caecimuris, or a metabolic pathway associated therewith.

In certain embodiments, methods described herein for classification of a disease state may comprise, or expressly does not comprise, detection and/or quantification of Blautia hominis, or a metabolic pathway associated therewith.

In certain embodiments, methods described herein for classification of a disease state may comprise, or expressly does not comprise, detection and/or quantification of Blautia obeum, or a metabolic pathway associated therewith.

In certain embodiments, methods described herein for classification of a disease state may comprise, or expressly does not comprise, detection and/or quantification of Blautia product, or a metabolic pathway associated therewith.

In certain embodiments, methods described herein for classification of a disease state may comprise, or expressly does not comprise, detection and/or quantification of Blautia stercoris, or a metabolic pathway associated therewith.

In certain embodiments, methods described herein for classification of a disease state may comprise, or expressly does not comprise, detection and/or quantification of Burkholderia ambifaria, or a metabolic pathway associated therewith.

In certain embodiments, methods described herein for classification of a disease state may comprise, or expressly does not comprise, detection and/or quantification of Burkholderia thailandensis, or a metabolic pathway associated therewith.

In certain embodiments, methods described herein for classification of a disease state may comprise, or expressly does not comprise, detection and/or quantification of Burkholderiaceae Burkholderia, or a metabolic pathway associated therewith.

In certain embodiments, methods described herein for classification of a disease state may comprise, or expressly does not comprise, detection and/or quantification of Burkholderiales Burkholderiaceae, or a metabolic pathway associated therewith.

In certain embodiments, methods described herein for classification of a disease state may comprise, or expressly does not comprise, detection and/or quantification of Burkholderiales Comamonadaceae, or a metabolic pathway associated therewith.

In certain embodiments, methods described herein for classification of a disease state may comprise, or expressly does not comprise, detection and/or quantification of Carnobacteriaceae Granulicatella, or a metabolic pathway associated therewith.

In certain embodiments, methods described herein for classification of a disease state may comprise, or expressly does not comprise, detection and/or quantification of Caulobacter segnis, or a metabolic pathway associated therewith.

In certain embodiments, methods described herein for classification of a disease state may comprise, or expressly does not comprise, detection and/or quantification of Caulobacteraceae Caulobacter, or a metabolic pathway associated therewith.

In certain embodiments, methods described herein for classification of a disease state may comprise, or expressly does not comprise, detection and/or quantification of Caulobacterales Caulobacteraceae, or a metabolic pathway associated therewith.

In certain embodiments, methods described herein for classification of a disease state may comprise, or expressly does not comprise, detection and/or quantification of Chloroplast Streptophyta, or a metabolic pathway associated therewith.

In certain embodiments, methods described herein for classification of a disease state may comprise, or expressly does not comprise, detection and/or quantification of Clostridiaceae Clostridium, or a metabolic pathway associated therewith.

In certain embodiments, methods described herein for classification of a disease state may comprise, or expressly does not comprise, detection and/or quantification of Clostridiaceae Hungatella, or a metabolic pathway associated therewith.

In certain embodiments, methods described herein for classification of a disease state may comprise, or expressly does not comprise, detection and/or quantification of Clostridiaceae Lactonifactor, or a metabolic pathway associated therewith.

In certain embodiments, methods described herein for classification of a disease state may comprise, or expressly does not comprise, detection and/or quantification of Clostridiales Clostridiaceae 1, or a metabolic pathway associated therewith.

In certain embodiments, methods described herein for classification of a disease state may comprise, or expressly does not comprise, detection and/or quantification of Clostridiales Incertae Sedis XI Parvimonas, or a metabolic pathway associated therewith.

In certain embodiments, methods described herein for classification of a disease state may comprise, or expressly does not comprise, detection and/or quantification of Clostridiales Monoglobus, or a metabolic pathway associated therewith.

In certain embodiments, methods described herein for classification of a disease state may comprise, or expressly does not comprise, detection and/or quantification of Clostridioides difficile, or a metabolic pathway associated therewith.

In certain embodiments, methods described herein for classification of a disease state may comprise, or expressly does not comprise, detection and/or quantification of Clostridium paraputrificum, or a metabolic pathway associated therewith.

In certain embodiments, methods described herein for classification of a disease state may comprise, or expressly does not comprise, detection and/or quantification of Clostridium sensu stricto, or a metabolic pathway associated therewith.

In certain embodiments, methods described herein for classification of a disease state may comprise, or expressly does not comprise, detection and/or quantification of Clostridium XIVa cluster, or a metabolic pathway associated therewith.

In certain embodiments, methods described herein for classification of a disease state may comprise, or expressly does not comprise, detection and/or quantification of Clostridium XI cluster, or a metabolic pathway associated therewith.

In certain embodiments, methods described herein for classification of a disease state may comprise, or expressly does not comprise, detection and/or quantification of Clostridium XVIII cluster, or a metabolic pathway associated therewith.

In certain embodiments, methods described herein for classification of a disease state may comprise, or expressly does not comprise, detection and/or quantification of Colidextribacter massiliensis, or a metabolic pathway associated therewith.

In certain embodiments, methods described herein for classification of a disease state may comprise, or expressly does not comprise, detection and/or quantification of Collinsella aerofaciens, or a metabolic pathway associated therewith.

In certain embodiments, methods described herein for classification of a disease state may comprise, or expressly does not comprise, detection and/or quantification of Coprococcus catus, or a metabolic pathway associated therewith.

In certain embodiments, methods described herein for classification of a disease state may comprise, or expressly does not comprise, detection and/or quantification of Coprococcus comes, or a metabolic pathway associated therewith.

In certain embodiments, methods described herein for classification of a disease state may comprise, or expressly does not comprise, detection and/or quantification of Coprococcus eutactus, or a metabolic pathway associated therewith.

In certain embodiments, methods described herein for classification of a disease state may comprise, or expressly does not comprise, detection and/or quantification of Coriobacteriaceae Atopobium, or a metabolic pathway associated therewith.

In certain embodiments, methods described herein for classification of a disease state may comprise, or expressly does not comprise, detection and/or quantification of Coriobacteriaceae Collinsella, or a metabolic pathway associated therewith.

In certain embodiments, methods described herein for classification of a disease state may comprise, or expressly does not comprise, detection and/or quantification of Coriobacteriaceae Eggerthella, or a metabolic pathway associated therewith.

In certain embodiments, methods described herein for classification of a disease state may comprise, or expressly does not comprise, detection and/or quantification of Coriobacteriales Coriobacteriaceae, or a metabolic pathway associated therewith.

In certain embodiments, methods described herein for classification of a disease state may comprise, or expressly does not comprise, detection and/or quantification of Corynebacteriaceae Corynebacterium, or a metabolic pathway associated therewith.

In certain embodiments, methods described herein for classification of a disease state may comprise, or expressly does not comprise, detection and/or quantification of Dorea formicigenerans, or a metabolic pathway associated therewith.

In certain embodiments, methods described herein for classification of a disease state may comprise, or expressly does not comprise, detection and/or quantification of Dorea longicatena, or a metabolic pathway associated therewith.

In certain embodiments, methods described herein for classification of a disease state may comprise, or expressly does not comprise, detection and/or quantification of Drancourtella massiliensis, or a metabolic pathway associated therewith.

In certain embodiments, methods described herein for classification of a disease state may comprise, or expressly does not comprise, detection and/or quantification of Eggerthella lenta, or a metabolic pathway associated therewith.

In certain embodiments, methods described herein for classification of a disease state may comprise, or expressly does not comprise, detection and/or quantification of Eggerthellaceae Eggerthella, or a metabolic pathway associated therewith.

In certain embodiments, methods described herein for classification of a disease state may comprise, or expressly does not comprise, detection and/or quantification of Enterobacterales Enterobacteriaceae, or a metabolic pathway associated therewith.

In certain embodiments, methods described herein for classification of a disease state may comprise, or expressly does not comprise, detection and/or quantification of Enterobacteriaceae Cedecea, or a metabolic pathway associated therewith.

In certain embodiments, methods described herein for classification of a disease state may comprise, or expressly does not comprise, detection and/or quantification of Enterobacteriaceae Citrobacter, or a metabolic pathway associated therewith.

In certain embodiments, methods described herein for classification of a disease state may comprise, or expressly does not comprise, detection and/or quantification of Enterobacteriaceae Escherichia, or a metabolic pathway associated therewith.

In certain embodiments, methods described herein for classification of a disease state may comprise, or expressly does not comprise, detection and/or quantification of Enterobacteriaceae Shimwellia, or a metabolic pathway associated therewith.

In certain embodiments, methods described herein for classification of a disease state may comprise, or expressly does not comprise, detection and/or quantification of Enterobacteriales Enterobacteriaceae, or a metabolic pathway associated therewith.

In certain embodiments, methods described herein for classification of a disease state may comprise, or expressly does not comprise, detection and/or quantification of Enterococcaceae Enterococcus, or a metabolic pathway associated therewith.

In certain embodiments, methods described herein for classification of a disease state may comprise, or expressly does not comprise, detection and/or quantification of Enterococcus saccharolyticus, or a metabolic pathway associated therewith.

In certain embodiments, methods described herein for classification of a disease state may comprise, or expressly does not comprise, detection and/or quantification of Erysipelatoclostridium [Clostridium] innocuum, or a metabolic pathway associated therewith.

In certain embodiments, methods described herein for classification of a disease state may comprise, or expressly does not comprise, detection and/or quantification of Erysipelatoclostridium ramosum, or a metabolic pathway associated therewith.

In certain embodiments, methods described herein for classification of a disease state may comprise, or expressly does not comprise, detection and/or quantification of Erysipelotrichaceae Erysipelatoclostridium, or a metabolic pathway associated therewith.

In certain embodiments, methods described herein for classification of a disease state may comprise, or expressly does not comprise, detection and/or quantification of Erysipelotrichaceae Faecalicoccus, or a metabolic pathway associated therewith.

In certain embodiments, methods described herein for classification of a disease state may comprise, or expressly does not comprise, detection and/or quantification of Erysipelotrichaceae Holdemania, or a metabolic pathway associated therewith.

In certain embodiments, methods described herein for classification of a disease state may comprise, or expressly does not comprise, detection and/or quantification of Erysipelotrichaceae Longicatena, or a metabolic pathway associated therewith.

In certain embodiments, methods described herein for classification of a disease state may comprise, or expressly does not comprise, detection and/or quantification of Erysipelotrichaceae Turicibacter, or a metabolic pathway associated therewith.

In certain embodiments, methods described herein for classification of a disease state may comprise, or expressly does not comprise, detection and/or quantification of Erysipelotrichales Erysipelotrichaceae, or a metabolic pathway associated therewith.

In certain embodiments, methods described herein for classification of a disease state may comprise, or expressly does not comprise, detection and/or quantification of Eubacterium [Eubacterium] eligens, or a metabolic pathway associated therewith.

In certain embodiments, methods described herein for classification of a disease state may comprise, or expressly does not comprise, detection and/or quantification of Eubacterium siraeum, or a metabolic pathway associated therewith.

In certain embodiments, methods described herein for classification of a disease state may comprise, or expressly does not comprise, detection and/or quantification of Eubacterium ventriosum, or a metabolic pathway associated therewith.

In certain embodiments, methods described herein for classification of a disease state may comprise, or expressly does not comprise, detection and/or quantification of Faecalibacterium prausnitzii, or a metabolic pathway associated therewith.

In certain embodiments, methods described herein for classification of a disease state may comprise, or expressly does not comprise, detection and/or quantification of Faecalimonas umbilicata, or a metabolic pathway associated therewith.

In certain embodiments, methods described herein for classification of a disease state may comprise, or expressly does not comprise, detection and/or quantification of Firmicutes Bacilli, or a metabolic pathway associated therewith.

In certain embodiments, methods described herein for classification of a disease state may comprise, or expressly does not comprise, detection and/or quantification of Firmicutes Clostridia, or a metabolic pathway associated therewith.

In certain embodiments, methods described herein for classification of a disease state may comprise, or expressly does not comprise, detection and/or quantification of Fusobacteriaceae Fusobacterium, or a metabolic pathway associated therewith.

In certain embodiments, methods described herein for classification of a disease state may comprise, or expressly does not comprise, detection and/or quantification of Fusobacterium nucleatum, or a metabolic pathway associated therewith.

In certain embodiments, methods described herein for classification of a disease state may comprise, or expressly does not comprise, detection and/or quantification of Gammaproteobacteria Enterobacterales, or a metabolic pathway associated therewith.

In certain embodiments, methods described herein for classification of a disease state may comprise, or expressly does not comprise, detection and/or quantification of Gemmiger, or a metabolic pathway associated therewith.

In certain embodiments, methods described herein for classification of a disease state may comprise, or expressly does not comprise, detection and/or quantification of Hungatella effluvia, or a metabolic pathway associated therewith.

In certain embodiments, methods described herein for classification of a disease state may comprise, or expressly does not comprise, detection and/or quantification of Klebsiella quasipneumoniae, or a metabolic pathway associated therewith.

In certain embodiments, methods described herein for classification of a disease state may comprise, or expressly does not comprise, detection and/or quantification of Lachnoclostridium [Clostridium] boltede, or a metabolic pathway associated therewith.

In certain embodiments, methods described herein for classification of a disease state may comprise, or expressly does not comprise, detection and/or quantification of Lachnospiraceae [Eubacterium] rectale, or a metabolic pathway associated therewith.

In certain embodiments, methods described herein for classification of a disease state may comprise, or expressly does not comprise, detection and/or quantification of Lachnospiraceae Anaerobutyricum, or a metabolic pathway associated therewith.

In certain embodiments, methods described herein for classification of a disease state may comprise, or expressly does not comprise, detection and/or quantification of Lachnospiraceae Anaerostipes, or a metabolic pathway associated therewith.

In certain embodiments, methods described herein for classification of a disease state may comprise, or expressly does not comprise, detection and/or quantification of Lachnospiraceae Blautia, or a metabolic pathway associated therewith.

In certain embodiments, methods described herein for classification of a disease state may comprise, or expressly does not comprise, detection and/or quantification of Lachnospiraceae Clostridium XIVa, or a metabolic pathway associated therewith.

In certain embodiments, methods described herein for classification of a disease state may comprise, or expressly does not comprise, detection and/or quantification of Lachnospiraceae Coprococcus, or a metabolic pathway associated therewith.

In certain embodiments, methods described herein for classification of a disease state may comprise, or expressly does not comprise, detection and/or quantification of Lachnospiraceae Dorea, or a metabolic pathway associated therewith.

In certain embodiments, methods described herein for classification of a disease state may comprise, or expressly does not comprise, detection and/or quantification of Lachnospiraceae Eisenbergiella, or a metabolic pathway associated therewith.

In certain embodiments, methods described herein for classification of a disease state may comprise, or expressly does not comprise, detection and/or quantification of Lachnospiraceae Eubacterium rectale group, or a metabolic pathway associated therewith.

In certain embodiments, methods described herein for classification of a disease state may comprise, or expressly does not comprise, detection and/or quantification of Lachnospiraceae Faecalimonas, or a metabolic pathway associated therewith.

In certain embodiments, methods described herein for classification of a disease state may comprise, or expressly does not comprise, detection and/or quantification of Lachnospiraceae Fusicatenibacter, or a metabolic pathway associated therewith.

In certain embodiments, methods described herein for classification of a disease state may comprise, or expressly does not comprise, detection and/or quantification of Lachnospiraceae Hungatella, or a metabolic pathway associated therewith.

In certain embodiments, methods described herein for classification of a disease state may comprise, or expressly does not comprise, detection and/or quantification of Lachnospiraceae Lachnoclostridium, or a metabolic pathway associated therewith.

In certain embodiments, methods described herein for classification of a disease state may comprise, or expressly does not comprise, detection and/or quantification of Lachnospiraceae Roseburia, or a metabolic pathway associated therewith.

In certain embodiments, methods described herein for classification of a disease state may comprise, or expressly does not comprise, detection and/or quantification of Lachnospiraceae Sellimonas, or a metabolic pathway associated therewith.

In certain embodiments, methods described herein for classification of a disease state may comprise, or expressly does not comprise, detection and/or quantification of Lactobacilluseae Lactobacillus, or a metabolic pathway associated therewith.

In certain embodiments, methods described herein for classification of a disease state may comprise, or expressly does not comprise, detection and/or quantification of Lactobacillus Enterococcaceae, or a metabolic pathway associated therewith.

In certain embodiments, methods described herein for classification of a disease state may comprise, or expressly does not comprise, detection and/or quantification of Lactobacillus Streptococcaceae, or a metabolic pathway associated therewith.

In certain embodiments, methods described herein for classification of a disease state may comprise, or expressly does not comprise, detection and/or quantification of Lactobacillus rogosde, or a metabolic pathway associated therewith.

In certain embodiments, methods described herein for classification of a disease state may comprise, or expressly does not comprise, detection and/or quantification of Leuconostocaceae Weissella, or a metabolic pathway associated therewith.

In certain embodiments, methods described herein for classification of a disease state may comprise, or expressly does not comprise, detection and/or quantification of Megasphaera micronuciformis, or a metabolic pathway associated therewith.

In certain embodiments, methods described herein for classification of a disease state may comprise, or expressly does not comprise, detection and/or quantification of Methanobacteriaceae Methanobrevibacter, or a metabolic pathway associated therewith.

In certain embodiments, methods described herein for classification of a disease state may comprise, or expressly does not comprise, detection and/or quantification of Methanobrevibacter smithii, or a metabolic pathway associated therewith.

In certain embodiments, methods described herein for classification of a disease state may comprise, or expressly does not comprise, detection and/or quantification of Methylophilaceae Methylophilus, or a metabolic pathway associated therewith.

In certain embodiments, methods described herein for classification of a disease state may comprise, or expressly does not comprise, detection and/or quantification of Micrococcaceae Rothia, or a metabolic pathway associated therewith.

In certain embodiments, methods described herein for classification of a disease state may comprise, or expressly does not comprise, detection and/or quantification of Oxalobacter formigenes, or a metabolic pathway associated therewith.

In certain embodiments, methods described herein for classification of a disease state may comprise, or expressly does not comprise, detection and/or quantification of Pasteurellaceae Rodentibacter, or a metabolic pathway associated therewith.

In certain embodiments, methods described herein for classification of a disease state may comprise, or expressly does not comprise, detection and/or quantification of Pasteurellales Pasteurellaceae, or a metabolic pathway associated therewith.

In certain embodiments, methods described herein for classification of a disease state may comprise, or expressly does not comprise, detection and/or quantification of Pectobacterium carotovorum, or a metabolic pathway associated therewith.

In certain embodiments, methods described herein for classification of a disease state may comprise, or expressly does not comprise, detection and/or quantification of Pelomonas aquatic, or a metabolic pathway associated therewith.

In certain embodiments, methods described herein for classification of a disease state may comprise, or expressly does not comprise, detection and/or quantification of Pelomonas aquatica, or a metabolic pathway associated therewith.

In certain embodiments, methods described herein for classification of a disease state may comprise, or expressly does not comprise, detection and/or quantification of Peptoniphilaceae Anaerococcus, or a metabolic pathway associated therewith.

In certain embodiments, methods described herein for classification of a disease state may comprise, or expressly does not comprise, detection and/or quantification of Peptoniphilaceae Finegoldia, or a metabolic pathway associated therewith.

In certain embodiments, methods described herein for classification of a disease state may comprise, or expressly does not comprise, detection and/or quantification of Peptoniphilaceae Peptoniphilus, or a metabolic pathway associated therewith.

In certain embodiments, methods described herein for classification of a disease state may comprise, or expressly does not comprise, detection and/or quantification of Peptostreptococcaceae Clostridioides, or a metabolic pathway associated therewith.

In certain embodiments, methods described herein for classification of a disease state may comprise, or expressly does not comprise, detection and/or quantification of Peptostreptococcaceae Clostridium XI, or a metabolic pathway associated therewith.

In certain embodiments, methods described herein for classification of a disease state may comprise, or expressly does not comprise, detection and/or quantification of Peptostreptococcaceae Intestinibacter, or a metabolic pathway associated therewith.

In certain embodiments, methods described herein for classification of a disease state may comprise, or expressly does not comprise, detection and/or quantification of Peptostreptococcaceae Peptostreptococcus, or a metabolic pathway associated therewith.

In certain embodiments, methods described herein for classification of a disease state may comprise, or expressly does not comprise, detection and/or quantification of Peptostreptococcaceae Romboutsia, or a metabolic pathway associated therewith.

In certain embodiments, methods described herein for classification of a disease state may comprise, or expressly does not comprise, detection and/or quantification of Peptostreptococcaceae Terrisporobacter, or a metabolic pathway associated therewith.

In certain embodiments, methods described herein for classification of a disease state may comprise, or expressly does not comprise, detection and/or quantification of Phascolarctobacterium faecium, or a metabolic pathway associated therewith.

In certain embodiments, methods described herein for classification of a disease state may comprise, or expressly does not comprise, detection and/or quantification of Phyllobacteriaceae Phyllobacterium, or a metabolic pathway associated therewith.

In certain embodiments, methods described herein for classification of a disease state may comprise, or expressly does not comprise, detection and/or quantification of Porphyromonadaceae Barnesiella, or a metabolic pathway associated therewith.

In certain embodiments, methods described herein for classification of a disease state may comprise, or expressly does not comprise, detection and/or quantification of Porphyromonadacede Odoribacter, or a metabolic pathway associated therewith.

In certain embodiments, methods described herein for classification of a disease state may comprise, or expressly does not comprise, detection and/or quantification of Porphyromonadacede Parabacteroides, or a metabolic pathway associated therewith.

In certain embodiments, methods described herein for classification of a disease state may comprise, or expressly does not comprise, detection and/or quantification of Prevotella copri, or a metabolic pathway associated therewith.

In certain embodiments, methods described herein for classification of a disease state may comprise, or expressly does not comprise, detection and/or quantification of Prevotellaceae Prevotella, or a metabolic pathway associated therewith.

In certain embodiments, methods described herein for classification of a disease state may comprise, or expressly does not comprise, detection and/or quantification of Prevotellamassilia timonensis, or a metabolic pathway associated therewith.

In certain embodiments, methods described herein for classification of a disease state may comprise, or expressly does not comprise, detection and/or quantification of Proteobacteria Gammaproteobacteria, or a metabolic pathway associated therewith.

In certain embodiments, methods described herein for classification of a disease state may comprise, or expressly does not comprise, detection and/or quantification of Rikenellaceae Alistipes, or a metabolic pathway associated therewith.

In certain embodiments, methods described herein for classification of a disease state may comprise, or expressly does not comprise, detection and/or quantification of Romboutsia timonensis, or a metabolic pathway associated therewith.

In certain embodiments, methods described herein for classification of a disease state may comprise, or expressly does not comprise, detection and/or quantification of Roseburia fuecis, or a metabolic pathway associated therewith.

In certain embodiments, methods described herein for classification of a disease state may comprise, or expressly does not comprise, detection and/or quantification of Roseburia intestinalis, or a metabolic pathway associated therewith.

In certain embodiments, methods described herein for classification of a disease state may comprise, or expressly does not comprise, detection and/or quantification of Roseburia inulinivorans, or a metabolic pathway associated therewith.

In certain embodiments, methods described herein for classification of a disease state may comprise, or expressly does not comprise, detection and/or quantification of Rosenbergiella collisarenosi, or a metabolic pathway associated therewith.

In certain embodiments, methods described herein for classification of a disease state may comprise, or expressly does not comprise, detection and/or quantification of Rosenbergiella nectarea, or a metabolic pathway associated therewith.

In certain embodiments, methods described herein for classification of a disease state may comprise, or expressly does not comprise, detection and/or quantification of Ruminococcaceae Anaeromassilibacillus, or a metabolic pathway associated therewith.

In certain embodiments, methods described herein for classification of a disease state may comprise, or expressly does not comprise, detection and/or quantification of Ruminococcaceae Clostridium IV cluster, or a metabolic pathway associated therewith.

In certain embodiments, methods described herein for classification of a disease state may comprise, or expressly does not comprise, detection and/or quantification of Ruminococcaceae Clostridium leptum group, or a metabolic pathway associated therewith.

In certain embodiments, methods described herein for classification of a disease state may comprise, or expressly does not comprise, detection and/or quantification of Ruminococcaceae Intestinimonas, or a metabolic pathway associated therewith.

In certain embodiments, methods described herein for classification of a disease state may comprise, or expressly does not comprise, detection and/or quantification of Ruminococcaceae Oscillibacter, or a metabolic pathway associated therewith.

In certain embodiments, methods described herein for classification of a disease state may comprise, or expressly does not comprise, detection and/or quantification of Ruminococcaceae Pseudoflavonifractor, or a metabolic pathway associated therewith.

In certain embodiments, methods described herein for classification of a disease state may comprise, or expressly does not comprise, detection and/or quantification of Ruminococcaceae Ruminococcus, or a metabolic pathway associated therewith.

In certain embodiments, methods described herein for classification of a disease state may comprise, or expressly does not comprise, detection and/or quantification of Ruminococcaceae Subdoligranulum, or a metabolic pathway associated therewith.

In certain embodiments, methods described herein for classification of a disease state may comprise, or expressly does not comprise, detection and/or quantification of Ruminococcus bromii, or a metabolic pathway associated therewith.

In certain embodiments, methods described herein for classification of a disease state may comprise, or expressly does not comprise, detection and/or quantification of Ruminococcus callidus, or a metabolic pathway associated therewith.

In certain embodiments, methods described herein for classification of a disease state may comprise, or expressly does not comprise, detection and/or quantification of Ruthenibacterium lactatiformans, or a metabolic pathway associated therewith.

In certain embodiments, methods described herein for classification of a disease state may comprise, or expressly does not comprise, detection and/or quantification of Saccharibacteria Incertae Sedis, or a metabolic pathway associated therewith.

In certain embodiments, methods described herein for classification of a disease state may comprise, or expressly does not comprise, detection and/or quantification of Salmonella enterica, or a metabolic pathway associated therewith.

In certain embodiments, methods described herein for classification of a disease state may comprise, or expressly does not comprise, detection and/or quantification of Solobacterium moorei, or a metabolic pathway associated therewith.

In certain embodiments, methods described herein for classification of a disease state may comprise, or expressly does not comprise, detection and/or quantification of Sphingobacteriaceae Pedobacter, or a metabolic pathway associated therewith.

In certain embodiments, methods described herein for classification of a disease state may comprise, or expressly does not comprise, detection and/or quantification of Sphingobacteriaceae Pedobacter, or a metabolic pathway associated therewith.

In certain embodiments, methods described herein for classification of a disease state may comprise, or expressly does not comprise, detection and/or quantification of Sphingomonadaceae, or a metabolic pathway associated therewith.

In certain embodiments, methods described herein for classification of a disease state may comprise, or expressly does not comprise, detection and/or quantification of Sphingomonas, or a metabolic pathway associated therewith.

In certain embodiments, methods described herein for classification of a disease state may comprise, or expressly does not comprise, detection and/or quantification of Staphylococcaceae Staphylococcus, or a metabolic pathway associated therewith.

In certain embodiments, methods described herein for classification of a disease state may comprise, or expressly does not comprise, detection and/or quantification of Streptococcaceae Streptococcus, or a metabolic pathway associated therewith.

In certain embodiments, methods described herein for classification of a disease state may comprise, or expressly does not comprise, detection and/or quantification of Streptococcus thermophilus, or a metabolic pathway associated therewith.

In certain embodiments, methods described herein for classification of a disease state may comprise, or expressly does not comprise, detection and/or quantification of Sutterellaceae Parasutterella, or a metabolic pathway associated therewith.

In certain embodiments, methods described herein for classification of a disease state may comprise, or expressly does not comprise, detection and/or quantification of Turicibacter sanguinis, or a metabolic pathway associated therewith.

In certain embodiments, methods described herein for classification of a disease state may comprise, or expressly does not comprise, detection and/or quantification of Veillonella dispar, or a metabolic pathway associated therewith.

In certain embodiments, methods described herein for classification of a disease state may comprise, or expressly does not comprise, detection and/or quantification of Veillonella infantium, or a metabolic pathway associated therewith.

In certain embodiments, methods described herein for classification of a disease state may comprise, or expressly does not comprise, detection and/or quantification of Veillonella parvula, or a metabolic pathway associated therewith.

In certain embodiments, methods described herein for classification of a disease state may comprise, or expressly does not comprise, detection and/or quantification of Veillonellaceae Dialister, or a metabolic pathway associated therewith.

In certain embodiments, methods described herein for classification of a disease state may comprise, or expressly does not comprise, detection and/or quantification of Veillonellaceae Veillonella, or a metabolic pathway associated therewith.

In certain embodiments, a binary delineation between disease classification of IBD and IBS comprises, consists of, or consists essentially of measuring the presence, absence, and/or relative quantity of at least or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 metabolic pathway features identified in Table 2.

In certain embodiments, a binary delineation between disease classification of IBD and CDI comprises, consists of, or consists essentially of measuring the presence, absence, and/or relative quantity of at least or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 metabolic pathway features identified in Table 3.

In certain embodiments, a binary delineation between disease classification of IBD (UC) and healthy comprises, consists of, or consists essentially of measuring the presence, absence, and/or relative quantity of at least or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 metabolic pathway features identified in Table 4.

In certain embodiments, a binary delineation between disease classification of IBD (CD) and healthy comprises, consists of, or consists essentially of measuring the presence, absence, and/or relative quantity of at least or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 metabolic pathway features identified in Table 5.

In certain embodiments, a binary delineation between disease classification of IBD (CD) and IBD (UC) comprises, consists of, or consists essentially of measuring the presence, absence, and/or relative quantity of at least or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 metabolic pathway features identified in Table 6.

In certain embodiments, a binary delineation between disease classification of IBS and CDI comprises, consists of, or consists essentially of measuring the presence, absence, and/or relative quantity of at least or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 metabolic pathway features identified in Table 7.

In certain embodiments, a binary delineation between disease classification of IBS and healthy comprises, consists of, or consists essentially of measuring the presence, absence, and/or relative quantity of at least or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 metabolic pathway features identified in Table 8.

In certain embodiments, a series of binary delineations are made to determine a final disease classification. For example but not limited to, delineation according to Table 3, followed by delineation according to Table 2, etc.

In certain embodiments, a binary delineation between disease classification of IBS and healthy comprises, consists of, or consists essentially of measuring the presence, absence, and/or relative quantity of at least or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 taxanomic features identified in Table 9.

In certain embodiments, a binary delineation between disease classification of IBS and CDI comprises, consists of, or consists essentially of measuring the presence, absence, and/or relative quantity of at least or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 taxanomic features identified in Table 10.

In certain embodiments, a binary delineation between disease classification of IBS and IBD comprises, consists of, or consists essentially of measuring the presence, absence, and/or relative quantity of at least or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 taxanomic features identified in Table 11.

In certain embodiments, a binary delineation between disease classification of IBD and CDI comprises, consists of, or consists essentially of measuring the presence, absence, and/or relative quantity of at least or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 taxanomic features identified in Table 12.

In certain embodiments, a binary delineation between disease classification of IBD UC and healthy comprises, consists of, or consists essentially of measuring the presence, absence, and/or relative quantity of at least or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 taxanomic features identified in Table 13.

In certain embodiments, a binary delineation between disease classification of IBD CD and healthy comprises, consists of, or consists essentially of measuring the presence, absence, and/or relative quantity of at least or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 taxanomic features identified in Table 14.

In certain embodiments, a binary delineation between disease classification of IBD CD and IBD UC comprises, consists of, or consists essentially of measuring the presence, absence, and/or relative quantity of at least or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 taxanomic features identified in Table 15.

In certain embodiments, a binary delineation between disease classification of pediatric CDI and pediatric healthy comprises, consists of, or consists essentially of measuring the presence, absence, and/or relative quantity of at least or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 taxanomic features identified in Table 16.

In certain embodiments, a binary delineation between disease classification of pediatric CDI and adult CDI comprises, consists of, or consists essentially of measuring the presence, absence, and/or relative quantity of at least or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 taxanomic features identified in Table 17.

In certain aspects, methods disclosed herein can relate to a system for performing such methods, the system comprising (a) apparatus or device for storing data regarding feature levels of one or more microbiome components; (b) apparatus or device for determining feature levels of at least one feature; (c) apparatus or device for comparing feature levels of a first feature with a predetermined first threshold value and/or test value; (d) apparatus or device for determining feature level of at least one second or more features; and (c) computing apparatus or device programmed to provide treatment with an appropriate methodology if the data indicates altered feature levels or activity of said first feature as compared to the predetermined first threshold value and/or test value, and, alternatively or in concert, expression level and/or activity of said second or more features as compared to the predetermined second or more feature threshold level and/or test value.

A person skilled in the art readily appreciates that an accurate prognosis can be given or determined if a sufficiently large number of feature levels are analyzed and compared to an appropriate control. In some embodiments, accurate prognosis can facilitate determination of disease recurrence and/or appropriate therapies to provide, including a particular therapy of any kind, such as an antibiotic therapy.

In some embodiments, feature levels and/or patterns can also be compared by using one or more ratios between feature abundance levels associated with an otherwise healthy microbiome and/or one or more dysbiosed microbiomes. Other suitable measures or indicators can also be employed for assessing the relationship or difference between different feature patterns.

In some embodiments, one or more of the features can be used to determine whether a patient with a diarrheal disorder should be treated with antimicrobials and/or antibiotics. In certain embodiments, a pattern of features in a patient fecal sample and/or other microbiome samples may be used to evaluate a patient to determine whether they are likely to respond to one or more therapeutic interventions. In some embodiments, likeliness of a therapeutic response for the patient may be considered with respect to an individual that lacks the particular feature pattern of the patient.

In some embodiments, a subject's (e.g., a patient's) feature levels can be compared to reference feature levels using various methods. In some embodiments, reference levels can be determined using expression levels of a reference based on otherwise healthy patients, all types of FGID patients, and/or all types of CDI, IBS, and/or IBD patients. In some embodiments, reference levels can be based on an internal reference such as a gene, metabolic pathway, and/or microbe that is present ubiquitously. In some embodiments, comparison can be performed using the fold change or the absolute difference between the feature levels to be compared. In some embodiments, one or more taxonomic and/or metabolic features can be used in the comparison.

In some embodiments, it is contemplated that 1, 2, 3, 4, 5, 6, 7, 8, 9, 10, 11, 12, 13, 14, 15, 16, 17, 18, 19, 20, 21, 22, 23, 24, and/or 25 features may be compared to each other and/or to a reference that is internal or external. In some embodiments, it is contemplated that 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, and/or 100 features may be compared to each other and/or to a reference that is internal or external. In some embodiments, it is contemplated that 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 1443, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 82, 183, 184, 185, 186, 187, 188, 189, 190, 191, 191, 192, 193, 194, 195, 196, 197, 198, 199, and/or 200 features may be compared to each other and/or to a reference that is internal or external. In some embodiments, it is contemplated that any number of features identified in Tables 1-19 may be compared to each other and/or to a reference that is internal or external.

In certain embodiments, comparisons or results from comparisons may reveal or be expressed as x-fold increase or decrease in expression relative to a standard or relative to another feature or relative to the same feature but in a different patient cohort (e.g., a disease patient and/or cohort compared to an appropriate health control). In some embodiments, patients with a particular disease diagnosis may have a relatively high level of feature presentation (e.g., over representation) or relatively low level of feature presentation (e.g., under representation) when compared to patients with a different disease diagnosis and/or otherwise healthy patients, or vice versa.

Fold increases or decreases may be, be at least, or be at most 1-, 2-, 3-, 4-, 5-, 6-, 7-, 8-, 9-, 10-, 11-, 12-, 13-, 14-, 15-, 16-, 17-, 18-, 19-, 20-, 25-, 30-, 35-, 40-, 45-, 50-, 55-, 60-, 65-, 70-, 75-, 80-, 85-, 90-, 95-, 100- or more, or any range derivable therein. Alternatively, differences in expression may be expressed as a percent decrease or increase, such as at least or at most 20, 25, 30, 35, 40, 45, 50, 55, 60, 65, 70, 75, 80, 85, 90, 95, 100, 110, 120, 130, 140, 150, 160, 170, 180, 190, 200, 300, 400, 500, 600, 700, 800, 900, 1000, or greater than 1000% difference, or any range derivable therein. In some embodiments, a fold level change for one or more features may not be calculatable, as one or more features may be absent in one or more disease and/or control patients and/or cohorts (e.g., dividing by zero).

Other ways to express relative expression levels are by normalized or relative numbers such as 0, 0.00001, 0.0001, 0.0002, 0.0003, 0.0004, 0.0005, 0.0006, 0.0007, 0.0008, 0.0009, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, or any range derivable therein.

In certain embodiments, a feature may be ranked in importance and/or otherwise identified according to a random forest feature rank mean decrease in accuracy. For example, a feature may be considered more integral for appropriate disease classification as a function of the random forest feature rank mean decrease in accuracy. In some embodiments, a higher random forest feature mean decrease in value means the feature has a greater potential disease classification value when compared to a feature with a lower value. In certain embodiments, a feature random forest feature rank mean decrease in accuracy may be 0.00001, 0.0001, 0.0002, 0.0003, 0.0004, 0.0005, 0.0006, 0.0007, 0.0008, 0.0009, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, or any range derivable therein.

In some embodiments, algorithms, such as the weighted voting programs, can be used to facilitate the evaluation of feature levels. In addition, in some embodiments, other clinical evidence can be combined with a feature-based test to reduce the risk of false evaluations. In some embodiments, other molecular based evaluations may be considered. In some embodiments, patient questionnaires may be considered. In some embodiments, patient medical histories may be considered. In some embodiments, patient endoscopy results may be considered.

In some embodiments, any biological sample from a patient that accurately represents the microbiome may be used to evaluate the presence, absence, and/or level of any feature discussed herein. In some embodiments, a biological sample from a fecal sample is used. In some embodiments, a biological sample from an endoscopy is used. In some embodiments, a biological sample from a mucosal biopsy is used. In some embodiments, a biological sample from intestinal fluid is used. Evaluation of a biological sample may involve, though it need not involve, panning (enriching) for microbiome components or isolation of specific microbes.

III. Methods of therapeutic intervention

In some embodiments, methods described herein are not limited to intestinal disorders, but are applicable to other microbiome dysbiosis associated disorders.

In certain embodiments, methods of treatment of intestinal disorders are based on features (e.g., taxa and/or metabolic pathways) identified by Taxa4Meta mediated diverse 16S data analysis.

In certain aspects, provided herein are methods for treating a subject determined to have CDI, IBS, IBD UC, and/or IBD CD based on a predetermined profile of one or more taxonomic and/or metabolic pathway features disclosed herein.

In certain embodiments, provided herein are methods for identifying features associated with diseases associated with microbiome dysbiosis, for example but not limited to CDI, IBS, IBD UC, IBD CD, antibiotic-associated diarrhea (AAD), celiac disease, food allergies, autoimmune disease, cancer, and/or graft versus host disease.

In some embodiments, an appropriate therapeutic agent is a small molecule, a biologic (e.g., an antibody, a recombinant protein, a cell therapy, etc.), a microbiota therapy (e.g., fecal transplant, fecal microbiota therapy, etc.), a mineral, a vitamin, a dietary restriction, a life style restriction and/or behavioral therapy.

In some embodiments, an appropriate therapeutic intervention for treating a subject that has received a CDI disease classification include but are not limited to administration of: vancomycin, fidaxomicin, bezlotoxumab, metronidazole (less preferred), fecal microbiota therapy (FMT) (e.g., particularly in cases of recurrent CDI), and/or microbiota consortia products.

In some embodiments, an appropriate therapeutic intervention for treating a subject that has received an IBD disease classification include but are not limited to administration of: anti-inflammatory drugs (e.g., for reduction of digestive tract inflammation), sulfasalazine, corticosteroids, immune suppressants (e.g., to prevent the autoimmune attacks), azathioprinc, antibiotics (e.g., to ameliorate bacterial infections), ciprofloxacin, metronidazole, TNF signaling pathway antagonists, Cimzia, α4β7 integrin antagonists, Entyvio (vedolizumab), Humira (adalimumab), Remicade (infliximab), Simponi (golimumab), Stelara (ustekinumab), anti-diarrheal agents (e.g., to prevent diarrhea and ameliorate associated symptoms), loperamide, diphenoxylate, cholestyramine, analgesics (e.g., to reduce pain and ameliorate associated symptoms), acetaminophen, vitamin and/or mineral supplements, vitamin D, and/or calcium.

In some embodiments, an appropriate therapeutic intervention for treating a subject that has received an IBS disease classification include but are not limited to administration of: bezlotoxumab, anti-diarrheal agents (e.g., to prevent diarrhea and/or ameliorate associated symptoms), loperamide, cholestyramine, colestipol, anticholinergics (e.g., to relieve spasms), dicyclomine, tricyclic antidepressants (e.g., to relieve depression and severe pain), imipramine, desipramine, selective serotonin reuptake inhibitors (SSRIs) (e.g., to relieve depression, pain and/or constipation), fluoxetine, paroxetine, anticonvulsants (e.g., to relieve pain and/or bloating), pregabalin, and/or gabapentin.

Therapy provided herein may comprise administration of a combination of therapeutic agents, such as for example, a first therapy (e.g., antimicrobials) and a second therapy (e.g., dietary restrictions). The therapies may be administered in any suitable manner known in the art. For example, the first and second treatment may be administered sequentially (at different times) or concurrently (at the same time).

In some aspects, the first therapy and the second therapy are administered substantially simultaneously. In some aspects, the first therapy and the second therapy are administered sequentially. In some aspects, the first therapy, the second therapy, and a third therapy are administered sequentially. In some aspects, the first therapy is administered before administering the second therapy. In some aspects, the first therapy is administered after administering the second therapy.

Aspects of the disclosure relate to compositions and methods comprising therapeutic compositions. The different therapies may be administered in one composition or in more than one composition, such as 2 compositions, 3 compositions, or 4 compositions. Various combinations of the agents may be employed.

Therapeutic agents of the disclosure may be administered by the same route of administration or by different routes of administration. In some aspects, the therapy is administered intravenously, intramuscularly, subcutaneously, topically, orally, transdermally, intraperitoneally, intraorbitally, by implantation, by inhalation, intrathecally, intraventricularly, or intranasally. In some aspects, the antibiotic is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some aspects, a unit dose comprises a single administrable dose.

The quantity to be administered, both according to number of treatments and unit dose, depends on the treatment effect desired. An effective dose (also “effective amount” or “therapeutically effective amount”) is understood to refer to an amount necessary to achieve a particular effect. In the practice in certain aspects, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In certain aspects, the effective dose of the pharmaceutical composition is one which can provide a blood level of about 1 μM to 150 μM. In another aspect, the effective dose provides a blood level of about 4 μM to 100 μM.; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 M; or about 25 μM to 50 μM; or about 50 μM to 150 μM; or about 50 μM to 100 μM (or any range derivable therein). In other aspects, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain aspects, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

In certain instances, it will be desirable to have multiple administrations of the composition, e.g., 2, 3, 4, 5, 6 or more administrations. The administrations can be at 1, 2, 3, 4, 5, 6, 7, 8, to 5, 6, 7, 8, 9, 10, 11, or 12 week intervals, including all ranges there between.

The phrases “pharmaceutically acceptable” or “pharmacologically acceptable” refer to molecular entities and compositions that do not produce an adverse, allergic, or other untoward reaction when administered to an animal or human. As used herein, “pharmaceutically acceptable carrier” includes any and all solvents, dispersion media, coatings, anti-bacterial and anti-fungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. Supplementary active ingredients, such as other anti-infective agents and vaccines, can also be incorporated into the compositions.

The active compounds can be formulated for parenteral administration, e.g., formulated for injection via the intravenous, intramuscular, subcutaneous, or intraperitoneal routes. Typically, such compositions can be prepared as either liquid solutions or suspensions; solid forms suitable for use to prepare solutions or suspensions upon the addition of a liquid prior to injection can also be prepared; and, the preparations can also be emulsified.

The pharmaceutical forms suitable for injectable use include sterile aqueous solutions or dispersions; formulations including, for example, aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The proteinaccous compositions may be formulated into a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

A pharmaceutical composition can include a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various anti-bacterial and anti-fungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various other ingredients enumerated above, as required, followed by filtered sterilization or an equivalent procedure. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Administration of the compositions will typically be via any common route. This includes, but is not limited to oral, or intravenous administration. Alternatively, administration may be by orthotopic, intradermal, subcutaneous, intramuscular, intraperitoneal, or intranasal administration. Such compositions would normally be administered as pharmaceutically acceptable compositions that include physiologically acceptable carriers, buffers or other excipients.

Upon formulation, solutions will be administered in a manner compatible with the dosage formulation and in such amount as is therapeutically or prophylactically effective. The formulations are easily administered in a variety of dosage forms, such as the type of injectable solutions described above.

Various combinations of antimicrobial and/or antibiotic agents or compounds may be employed, for example an antimicrobial is “A” and an additional therapeutic agent is “B” (or a combination of such agents and/or compounds), and given as part of a therapeutic regimen, for example:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of a therapeutic compounds or agents to a patient will follow general protocols for the administration of such compounds, taking into account the toxicity, if any, of a therapy. It is expected that treatment cycles would be repeated as necessary. It also is contemplated that various standard therapies, as well as surgical intervention, may be applied in combination with a described therapy.

### IV. Kits

In some embodiments, the present invention also concern kits containing compositions of the disclosure or compositions to implement methods of the disclosure. In some aspects, kits can be used to evaluate one or more biomarkers (e.g., features as described herein). In some aspects, kits can be used to detect, for example, absence, presence, and/or level of one or more features described herein. In certain aspects, a kit contains, contains at least or contains at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57,58, 59, 60, 61, 62, 63, 64, 65, 66, 100, 132, 500, 1,000 or more probes, primers or primer sets, synthetic molecules or inhibitors, or any value or range and combination derivable therein.

In some embodiments, a kit can be prepared from readily available materials and reagents. For example, such kits can comprise any one or more of the following materials: enzymes, reaction tubes, buffers, detergent, primers, probes, antibodies. In some embodiments, a kit allows a practitioner to obtain biological samples. In another preferred embodiment these kits include the needed apparatus for performing RNA extraction, RT-PCR, oligonucleotide quantification, protein and/or metabolite quantification, and/or gel electrophoresis. Instructions for performing associated assays can also be included in a kit.

In some embodiments, a kit may comprise a number of agents for assessing differential levels and/or expression of a number of features, for example, at least one feature listed in Tables 1-19.

In some embodiments, a kit may comprise reagents for detection of 1, 2, 3, 4, 5, 6, 7, 8, 9, 10, 11, 12, 13, 14, 15, 16, 17, 18, 19, 20, 21, 22, 23, 24 and/or 25 features. In some embodiments, a kit may comprise reagents for detection of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, and/or 100 features. In some embodiments, a kit may comprise reagents for detection of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 1443, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 82, 183, 184, 185, 186, 187, 188, 189, 190, 191, 191, 192, 193, 194, 195, 196, 197, 198, 199, and/or 200 or more features.

In some embodiments, a kit is housed in a container. Kits may further comprise instructions for using the kit for assessing expression, means for converting the expression data into expression values and/or means for analyzing expression values to generate prognosis. Agents in a kit for measuring biomarker expression may comprise a plurality of PCR probes and/or primers for qRT-PCR and/or a plurality of antibody or fragments thereof for assessing expression of biomarkers. In another embodiment, agents in a kit for measuring biomarker expression may comprise an array of polynucleotides complementary to mRNAs of biomarkers identified herein. Possible means for converting expression data into expression values and for analyzing expression values to generate scores that predict survival or prognosis may be also included.

In some embodiments, a kit may comprise components, which may be individually packaged or placed in a container, such as a tube, bottle, vial, syringe, or other suitable container means.

Individual components may also be provided in a kit in concentrated amounts; in some aspects, a component is provided individually in the same concentration as it would be in a solution with other components. Concentrations of components may be provided as 1×, 2×, 5×, 10×, or 20× or more.

Kits for using probes, synthetic nucleic acids, nonsynthetic nucleic acids, and/or inhibitors of the disclosure for prognostic or diagnostic applications are included as part of the disclosure. Specifically contemplated are any such molecules corresponding to any biomarker identified herein, which includes nucleic acid primers/primer sets and probes that are identical to or complementary to all or part of a biomarker, which may include noncoding sequences of the biomarker, as well as coding sequences of the biomarker.

In certain aspects, negative and/or positive control nucleic acids, probes, and inhibitors are included in some kit aspects. In addition, a kit may include a sample that is a negative or positive control for copy number or expression of one or more biomarkers.

Any aspect of the disclosure involving specific taxanomic profile and/or metabolic pathway biomarker by name is contemplated also to cover aspects involving biomarkers whose characteristics are at least 80, 81, 82, 83, 84, 85, 86, 87, 88, 89, 90, 91, 92, 93, 94, 95, 96, 97, 98, or 99% identical to the specified taxanomic profile and/or metabolic pathway.

### V. Aspects

The following aspects describe certain inventions disclosed herein.
Aspect 1) A method comprising: applying 16S rRNA variable region-specific amplicon sequence length confidence threshold levels to a meta-analysis 16S rRNA sequencing dataset (e.g., pan-microbiome dataset) comprising sequences derived from diverse 16S rRNA sequencing data, wherein taxonomic misclassification rates are reduced when compared to taxonomic classification produced through global conservative confidence threshold application to all types of 16S amplicon data, and wherein the amplicon sequence length is measured in number of bases in an amplicon.
Aspect 2) The method of aspect 1, wherein the region-specific amplicon sequence length levels are: ≥200 for V1V3 amplicon forward and ≥300 for V1V3 amplicon reverse; ≥200 for V1V2 amplicon forward and ≥300 for V1V2 amplicon reverse; >200 for V2V3 amplicon forward and ≥300 for V2V3 amplicon reverse; ≥250 for V3V5 amplicon forward and ≥300 for V3V5 amplicon reverse; ≥250 for V3V4 amplicon forward and ≥300 for V3V4 amplicon reverse; ≥250 for V4V5 amplicon forward and ≥300 for V4V5 amplicon reverse; ≥200 for V4 amplicon forward and ≥200 for V4 amplicon reverse; and 2300 for V6V9 amplicon forward and ≥250 for V6V9 amplicon reverse, optionally, wherein the levels are: 200-450 for V1V3 amplicon forward and 300-450 for V1V3 amplicon reverse; 200-450 for V1V2 amplicon forward and 300-450 for V1V2 amplicon reverse; 200-450 for V2V3 amplicon forward and 300-450 for V2V3 amplicon reverse; 250-450 for V3V5 amplicon forward and 300-450 for V3V5 amplicon reverse; 250-450 for V3V4 amplicon forward and 300-450 for V3V4 amplicon reverse; 250-450 for V4V5 amplicon forward and 300-450 for V4V5 amplicon reverse; 200-250 for V4 amplicon forward and 200-250 for V4 amplicon reverse; and 300-450 for V6V9 amplicon forward and 250-450 for V6V9 amplicon reverse.
Aspect 3) The method of aspect 1 or 2, wherein unstitched forward reads after paired-end merging are included in the dataset to avoid discarding reads with good sequence quality, VSEARCH is used to cluster the data and 99% similarity is applied to defined length ranges in forward and reverse amplicon reads from selected 16S regions, and confident species calls are archived for further downstream use using Bayesian LCA-based Taxonomic Classification Method (BLCA) with stringent alignment.
Aspect 4) The method of any one of aspects 1-3, wherein stringent alignment parameters of 99% identity and 99% coverage are utilized.
Aspect 5) The method of any one of aspects 1-4, wherein the taxonomic confidence threshold selections at genus threshold are: 90 for V1V3 amplicon forward, 90 for V1V3 amplicon reverse; 90 for V1V2 amplicon forward, 90 for V1V2 amplicon reverse; 90 for V2V3 amplicon forward, 90 for V2V3 amplicon reverse; 85 for V3V5 amplicon forward, 85 for V3V5 amplicon reverse; 85 for V3V4 amplicon forward, 85 for V3V4 amplicon reverse; 85 for V4V5 amplicon forward, 85 for V4V5 amplicon reverse; 70 for V4 amplicon forward, 70 for V4 amplicon reverse; and/or 90 for V6V9 amplicon forward, 90 for V6V9 amplicon reverse; and/or wherein the taxonomic confidence threshold selection at species threshold are: 60 for V1V3 amplicon forward, 60 for V1V3 amplicon reverse; 60 for V1V2 amplicon forward, 60 for V1V2 amplicon reverse; 60 for V2V3 amplicon forward, 60 for V2V3 amplicon reverse; 50 for V3V5 amplicon forward, 50 for V3V5 amplicon reverse; 50 for V3V4 amplicon forward, 50 for V3V4 amplicon reverse; 50 for V4V5 amplicon forward, 50 for V4V5 amplicon reverse; 50 for V4 amplicon forward, 50 for V4 amplicon reverse; and/or 50 for V6V9 amplicon forward, 50 for V6V9 amplicon reverse.
Aspect 6) The method of any one of aspects 1-5, wherein the method is used to conduct population-scale meta-analysis to define a microbiome.
Aspect 7) The method of any one of aspects 1-6, wherein the method is used to conduct population scale meta-analysis to define a healthy human gut microbiome.
Aspect 8) The method of any one of aspects 1-7, wherein the method is used to conduct population-scale meta-analysis to define microbiome dysbiosis (i.e. non-healthy) in a human gut microbiome, wherein the non-healthy human gut microbiome is associated with irritable bowel syndrome (IBS), inflammatory bowel diseases (IBD), or Clostridiodes difficile infection (CDI).
Aspect 9) The method of aspect 8, wherein features representative of microbiome dysbiosis associated with IBS, IBD Ulcerative Colitis (UC), IBD Crohn's Disease (CD), and/or CDI are identified.
Aspect 10) The method of aspect 9, wherein the features associated with IBS, IBD UC. IBD CD, and/or CDI are used for disease classification.
Aspect 11) The method of aspect 10, wherein a patient with microbiome dysbiosis is diagnosed as having IBS, IBD UC, IBD CD, and/or CDI.
Aspect 12) The method of aspect 11, wherein a treatment regimen is adjusted or maintained according to the diagnosis.
Aspect 13) A method of treating an individual having diarrhea comprising: measuring for one or more taxonomical features from a biological sample from the individual; and reducing the administration of antibiotics and/or antimicrobial treatment to the individual when the individual has presence or absence or a certain level of one or more feature(s) indicative of non-CDI causative diarrhea, or administering antibiotics and/or antimicrobial treatment to the individual when the individual has presence or absence or a certain level of one or more feature(s) indicative of pathogenic infection.
Aspect 14) The method of aspect 13, wherein the antibiotics and/or antimicrobial treatment comprise at least one of the antibiotics selected from a small molecule antibiotic, an antibiotic derived from a natural product, a microbial composition, an antibody suitable for neutralizing pathogenic infections, a therapeutic, contact isolation, and any combination thereof.
Aspect 15) The method of aspect 14, with the proviso that if the non-CDI causative diarrhea is irritable bowel syndrome (IBS), administration of the antibiotic and/or antimicrobial rifaximin is not reduced.
Aspect 16) The method of aspect 14, wherein the antibiotics and/or antimicrobial treatment comprises at least one of vancomycin, fidaxomicin, and bezlotoxumab
Aspect 17) The method of aspect 16, wherein the treatment is fidaxomicin, and optionally the treatment dosage is at least 200 mg twice daily for 10 days, the treatment is vancomycin, and optionally the treatment dosage is at least 125 mg four times per day for 10 days, and/or the treatment is bezlotoxumab.
Aspect 18) The method of any one of aspects 13-17, wherein the pathogenic diarrhea classification or non-CDI causative diarrhea classification is characterized by measuring the presence, absence, and/or relative quantity of at least 10 taxonomical features described in any one of Tables 9-17
Aspect 19) The method of aspect 18, wherein the pathogenic diarrhea classification or non-CDI causative diarrhea classification is characterized by measuring the presence, absence, and/or relative quantity of at least 20 taxonomical features described in any one of Tables 9-17.
Aspect 20) The method of aspect 18, wherein the pathogenic diarrhea classification or non-CDI causative diarrhea classification is characterized by measuring the presence, absence, and/or relative quantity of at least 40 taxonomical features described in any one of Tables 9-17.
Aspect 21) The method of aspect 18, wherein the pathogenic diarrhea classification or non-CDI causative diarrhea classification is characterized by measuring the presence, absence, and/or relative quantity of at least 60 taxonomical features described in any one of Tables 9-17.
Aspect 22) The method of aspect 18, wherein the pathogenic diarrhea classification or non-CDI causative diarrhea classification is characterized by measuring the presence, absence, and/or relative quantity of at least 80 taxonomical features described in any one of Tables 9-17.
Aspect 23) The method of aspect 18, wherein the pathogenic diarrhea classification or non-CDI causative diarrhea classification is characterized by measuring the presence, absence, and/or relative quantity of at least 100 taxonomical features described in any one Tables 9-17.
Aspect 24) The method of any one of aspects 19-23, wherein the pathogenic diarrhea classification or non-CDI causative diarrhea classification comprises characterization using more than one of Tables 9-17.
Aspect 25) The method of aspect 24, wherein the more than one characterization using Tables 9-17 is sequential.
Aspect 26) The method of aspect 24 or 25, wherein the more than one characterization using Tables 9-17 comprises first characterizing using Tables 10 and/or 12, followed by characterization using one or more of the remaining Tables.
Aspect 27) The method of any one of aspects 13-26, wherein the measuring of one or more taxonomical features comprises at least one of analyzing one or more nucleic acids in the sample, analyzing one or more metabolites in the sample, and analyzing one or more proteins in the sample.
Aspect 28) The method of aspect 27, wherein the nucleic acid is analyzed by sequencing, polymerase chain reaction, isothermal amplification, bioinformatics, or any combination thereof.
Aspect 29) The method of aspect 28, wherein the nucleic acid analyzed is 16S ribosomal RNA.
Aspect 30) The method of aspect 27, wherein the metabolites are analyzed by mass spectrometry, ELISA, chromatography, or any combination thereof.
Aspect 31) The method of aspect 27, wherein the proteins are analyzed by mass spectrometry, ELISA, chromatography, Western blotting, immunoprecipitation, immunoelectrophoresis, or any combination thereof.
Aspect 32) The method of any one of aspects 13-31, wherein when reducing the administration of antibiotics and/or antimicrobial treatment to the individual when the individual has presence or absence or a certain level of one or more feature(s) indicative of non-CDI causative diarrhea, the subject microbiome is further characterized to determine whether the non-CDI causative diarrhea is associated with irritable bowel syndrome (IBS) or inflammatory bowel disease (IBD), and treatment is modified accordingly.
Aspect 33) The method of aspect 32, wherein the non-CDI causative diarrhea is associated with IBD, the IBD is further characterized to determine whether the IBD is Ulcerative Colitis (UC) or Crohn's Disease (CD), and treatment is modified accordingly.
Aspect 34) The method of aspect 13, wherein the measuring of one or more taxonomical features from a biological sample from the individual comprises determining changes in relative abundance, compared to a reference healthy gut microbiome, of at least one of: Sphingobacteriaceae Pedobacter; Saccharibacteria Incertae Sedis; Peptostreptococcaceae Intestinibacter; Bacillales Incertae Sedis Gemella; Veillonella infantium; Chloroplast Streptophyta; Caulobacter segnis; Methylophilaceae Methylophilus; Lactobacillus Streptococcaceae; Burkholderiales Comamonadaceae; Burkholderia ambifaria; Caulobacteraceae Caulobacter; Betaproteobacteria; Phyllobacteriaceae Phyllobacterium; Burkholderiales Burkholderiaceae; Sphingomonadaceae; Sphingomonas; Prevotellamassilia timonensis; Collinsella aerofaciens; Adlercreutzia equolifaciens; Blautia hominis; and Dorea formicigenerans; wherein the change in taxonomical feature relative abundance is indicative of non-CDI causative diarrhea associated with IBS.
Aspect 35) The method of aspect 34, wherein an increase in relative abundance of at least one of: Sphingobacteriaceae Pedobacter; Saccharibacteria Incertae Sedis; Peptostreptococcaceae Intestinibacter; Bacillales Incertae Sedis Gemella; Veillonella infantium; Chloroplast Streptophyta; Caulobacter segnis; Methylophilaceae Methylophilus; Lactobacillus Streptococcaceae; Burkholderiales Comamonadaceae; Burkholderia ambifaria; Caulobacteraceae Caulobacter; Betaproteobacteria; Phyllobacteriaceae Phyllobacterium; Burkholderiales Burkholderiaceae; Sphingomonadaceae; Sphingomonas; and Prevotellamassilia timonensis, is indicative of non-CDI causative diarrhea associated with IBS.
Aspect 36) The method of aspect 34, wherein a decrease in relative abundance of at least one of: Collinsella aerofaciens; Adlercreutzia equolifaciens; Blautia hominis; and Dorea formicigenerans, is indicative of non-CDI causative diarrhea associated with IBS.
Aspect 37) The method of aspect 34-36, wherein a change in relative abundance of at least four taxonomical features is indicative of non-CDI causative diarrhea associated with IBS.
Aspect 38) The method of aspect 13, wherein the measuring of one or more taxonomical features from a biological sample from the individual comprises determining changes in relative abundance, compared to a reference healthy gut microbiome, of at least one of: Acidaminococcaceae Acidaminococcus; Actinomycetales Actinomycetaceae; Bacillales Gemella; Bacilli Lactobacillus; Betaproteobacteria; Betaproteobacteria Burkholderiales; Bifidobacterium boum; Blautia hominis; Clostridiales Incertae Sedis XI Parvimonas; Enterobacteriaceae Cedecea; Erysipelotrichaceae Faecalicoccus; Faecalimonas umbilicata; Fusobacteriaceae Fusobacterium; Fusobacterium nucleatum; Gammaproteobacteria Enterobacterales; Lachnoclostridium [Clostridium] bolteae; Lachnospiraceae Lachnoclostridium; Megasphaera micronuciformis; Peptoniphilaceae Peptoniphilus; Peptostreptococcaceae Peptostreptococcus; Peptostreptococcaceae Romboutsia; Peptostreptococcaceae Terrisporobacter; Proteobacteria Gammaproteobacteria; Rosenbergiella collisarenosi; Rosenbergiella nectarea; Ruminococcaceae Intestinimonas; Ruminococcaceae Subdoligranulum; Solobacterium moorei; Veillonellaceae Veillonella; Alistipes shahii; Bacteroides cellulosilyticus; Bacteroides koreensis; Bacteroides thetaiotaomicron; Bacteroides xylanisolvens; Colidextribacter massiliensis; Coprococcus catus; Coprococcus eutactus; Enterobacterales Enterobacteriaceae; Faecalibacterium prausnitzii; Methanobrevibacter smithii; Phascolarctobacterium faecium; Romboutsia timonensis; and Turicibacter sanguinis; wherein the change in taxonomical feature relative abundance is indicative of non-CDI causative diarrhea associated with IBD.
Aspect 39) The method of aspect 38, wherein an increase in relative abundance of at least one of: Acidaminococcaceae Acidaminococcus; Actinomycetales Actinomycetaceae; Bacillales Gemella; Bacilli Lactobacillus; Betaproteobacteria; Betaproteobacteria Burkholderiales; Bifidobacterium boum; Blautia hominis; Clostridiales Incertae Sedis XI Parvimonas; Enterobacteriaceae Cedecea; Erysipelotrichaceae Faecalicoccus; Faecalimonas umbilicata; Fusobacteriaceae Fusobacterium; Fusobacterium nucleatum; Gammaproteobacteria Enterobacterales; Lachnoclostridium [Clostridium] boltede; Lachnospiraceae Lachnoclostridium; Megasphaera micronuciformis; Peptoniphilaceae Peptoniphilus; Peptostreptococcaceae Peptostreptococcus; Peptostreptococcaceae Romboutsia; Peptostreptococcaceae Terrisporobacter; Proteobacteria Gammaproteobacteria; Rosenbergiella collisarenosi; Rosenbergiella nectarea; Ruminococcaceae Intestinimonas; Ruminococcaceae Subdoligranulum; Solobacterium moorei; and Veillonellaceae Veillonella, is indicative of non-CDI causative diarrhea associated with IBD.
Aspect 40) The method of aspect 38, wherein a decrease in relative abundance of at least one of: Alistipes shahii; Bacteroides cellulosilyticus; Bacteroides koreensis; Bacteroides thetaiotaomicron; Bacteroides xylanisolvens; Colidextribacter massiliensis; Coprococcus catus; Coprococcus eutactus; Enterobacterales Enterobacteriaceae; Faecalibacterium prausnitzii; Methanobrevibacter smithii; Phascolarctobacterium faecium; Romboutsia timonensis; and Turicibacter sanguinis, is indicative of non-CDI causative diarrhea associated with IBD.
Aspect 41) The method of aspect 38-40, wherein a change in relative abundance of at least four taxonomical features is indicative of non-CDI causative diarrhea associated with IBD.
Aspect 42) The method of aspect 13, wherein the individual is a pediatric individual, and wherein the measuring of one or more taxonomical features from a biological sample from the individual comprises determining changes in relative abundance, compared to a reference healthy pediatric gut microbiome, of at least one of: Bacilli Lactobacillus; Peptostreptococcaceae Clostridioides; Enterococcaceae Enterococcus; Eggerthellaceae Eggerthella; Erysipelotrichaceae Erysipelatoclostridium; Lachnospiraceae Lachnoclostridium; Firmicutes Bacilli; Enterobacteriaceae Escherichia; Enterobacteriales Enterobacteriaceae; Streptococcaceae Streptococcus; Actinomycetaceae Schaalia; Clostridiaceae Hungatella; Clostridiaceae Clostridium; Corynebacteriaceae Corynebacterium; Veillonellaceae Veillonella; Actinomycetaceae Actinomyces; Fusobacteriaceae Fusobacterium; Erysipelotrichaceae Longicatena; Lachnospiraceae Clostridium XIVa; Lachnospiraceae Eisenbergiella; Peptostreptococcaceae Intestinibacter; Enterobacteriaceae Citrobacter; Peptoniphilaceae Finegoldia; Carnobacteriaceae Granulicatella; Coriobacteriaceae Eggerthella; Peptostreptococcaceae Peptostreptococcus; Drancourtella massiliensis; Peptoniphilaceae Anaerococcus; Bacillales Gemella; Lachnospiraceae Sellimonas; Peptoniphilaceae Peptoniphilus; Pasteurellaceae Rodentibacter; Clostridium sensu stricto; Lachnospiraceae Faecalimonas; Proteobacteria Gammaproteobacteria; Clostridiaceae Lactonifactor; Micrococcaceae Rothia; Leuconostocaceae Weissella; Peptostreptococcaceae Terrisporobacter; Peptostreptococcaceae Romboutsia; Peptostreptococcaceae Clostridium XI; Coriobacteriaceae Atopobium; Atopobiaceae Atopobium; Ruminococcaceae Anaeromassilibacillus; Porphyromonadaceae Parabacteroides; Rikenellaceae Alistipes; Lachnospiraceae Eubacterium rectale group; Lachnospiraceae Coprococcus; Lachnospiraceae Roseburia; Porphyromonadacede Odoribacter; Veillonellaceae Dialister; Ruminococcaceae Clostridium IV; Bacteroidia Bacteroidales; Coriobacteriaceae Collinsella; Coriobacteriales Coriobacteriaceae; Ruminococcaceae Clostridium leptum group; Enterobacteriaceae Shimwellia; Acidaminococcaceae Phascolarctobacterium; Staphylococcaceae Staphylococcus; Lachnospiraceae Anaerobutyricum; Erysipelotrichaceae Holdemania; Clostridiales Monoglobus; Porphyromonadaceae Barnesiella; Pasteurellales Pasteurellaceae; Clostridiales Clostridiaceae 1; and Bacteroidales Rikenellaceae; wherein the change in taxonomical feature relative abundance is indicative of CDI associated diarrhea.
Aspect 43) The method of aspect 42, wherein an increase in relative abundance of at least one of: Bacilli Lactobacillus; Peptostreptococcaceae Clostridioides; Enterococcaceae Enterococcus; Eggerthellaceae Eggerthella; Erysipelotrichaceae Erysipelatoclostridium; Lachnospiraceae Lachnoclostridium; Firmicutes Bacilli; Enterobacteriaceae Escherichia; Enterobacteriales Enterobacteriaceae; Streptococcaceae Streptococcus; Actinomycetaceae Schaalia; Clostridiaceae Hungatella; Clostridiaceae Clostridium; Corynebacteriaceae Corynebacterium; Veillonellaceae Veillonella; Actinomycetaceae Actinomyces; Fusobacteriaceae Fusobacterium; Erysipelotrichaceae Longicatena; Lachnospiraceae Clostridium XIVa; Lachnospiraceae Eisenbergiella; Peptostreptococcaceae Intestinibacter; Enterobacteriaceae Citrobacter; Peptoniphilaceae Finegoldia; Carnobacteriaceae Granulicatella; Coriobacteriaceae Eggerthella; Peptostreptococcaceae Peptostreptococcus; Drancourtella massiliensis; Peptoniphilaceae Anaerococcus; Bacillales Gemella; Lachnospiraceae Sellimonas; Peptoniphilaceae Peptoniphilus; Pasteurellaceae Rodentibacter; Clostridium sensu stricto; Lachnospiraceae Faecalimonas; Proteobacteria Gammaproteobacteria; Clostridiaceae Lactonifactor; Micrococcaceae Rothia; Leuconostocaceae Weissella; Peptostreptococcaceae Terrisporobacter; Peptostreptococcaceae Romboutsia; Peptostreptococcaceae Clostridium XI; Coriobacteriaceae Atopobium; Atopobiaceae Atopobium; and Ruminococcaceae Anaeromassilibacillus, is indicative of CDI associated diarrhea.
Aspect 44) The method of aspect 42, wherein a decrease in relative abundance of at least one of: Porphyromonadaceae Parabacteroides; Rikenellaceae Alistipes; Lachnospiraceae Eubacterium rectale group; Lachnospiraceae Coprococcus; Lachnospiraceae Roseburia; Porphyromonadacede Odoribacter; Veillonellaceae Dialister; Ruminococcaceae Clostridium IV; Bacteroidia Bacteroidales; Coriobacteriaceae Collinsella; Coriobacteriales Coriobacteriaceae; Ruminococcaceae Clostridium leptum group; Enterobacteriaceae Shimwellia; Acidaminococcaceae Phascolarctobacterium; Staphylococcaceae Staphylococcus; Lachnospiraceae Anaerobutyricum; Erysipelotrichaceae Holdemania; Clostridiales Monoglobus; Porphyromonadaceae Barnesiella; Pasteurellales Pasteurellaceae; Clostridiales Clostridiaceae 1; and Bacteroidales Rikenellaceae, is indicative of CDI associated diarrhea.
Aspect 45) The method of aspect 42-44, wherein a change in relative abundance of at least four taxonomical features is indicative of CDI associated diarrhea.
Aspect 46) The method of aspect 32, wherein the characterization of the subject microbiome to determine whether the non-CDI causative diarrhea is associated with IBS or IBD comprises measuring one or more taxonomical features from a biological sample from the subject and determining changes in relative abundance, compared to a reference dysbiosis IBD gut microbiome, of at least one of: Blautia stercoris; Saccharibacteria Incertae Sedis; Bacteroides plebeius; Bacteroides nordii; Eubacterium siraeum; Bacteroides cellulosilyticus; Burkholderiales Burkholderiaceae; Caulobacteraceae Caulobacter; Pelomonas aquatic; Burkholderiaceae Burkholderia; Caulobacter segnis; Burkholderia ambifaria; Eubacterium ventriosum; Firmicutes Clostridia; Oxalobacter formigenes; Burkholderiales Comamonadaceae; Veillonella infantium; Bacteroides thetaiotaomicron; Sphingobacteriaceae Pedobacter; Burkholderia thailandensis; Erysipelotrichaceae Turicibacter; Collinsella aerofaciens; Veillonellaceae Veillonella; Lachnospiraceae Lachnoclostridium; Blautia hominis; Gammaproteobacteria Enterobacterales; Bifidobacteriales Bifidobacteriaceae; Ruminococcaceae Intestinimonas; Faecalimonas umbilicata; Actinomycetales Actinomycetaceae; Actinobacteria Actinobacteria; Dorea formicigenerans; and Bacteria Proteobacteria; wherein the change in taxonomical feature relative abundance is indicative of non-CDI causative diarrhea associated with IBS.
Aspect 47) The method of aspect 46, wherein an increase in relative abundance of at least one of: Saccharibacteria Incertae Sedis; Bacteroides plebeius; Bacteroides nordii; Eubacterium siraeum; Bacteroides cellulosilyticus; Burkholderiales Burkholderiaceae; Caulobacteraceae Caulobacter; Pelomonas aquatic; Burkholderiaceae Burkholderia; Caulobacter segnis; Burkholderia ambifaria; Eubacterium ventriosum; Firmicutes Clostridia; Oxalobacter formigenes; Burkholderiales Comamonadaceae; Veillonella infantium; Bacteroides thetaiotaomicron; Sphingobacteriaceae Pedobacter; Burkholderia thailandensis; and Erysipelotrichaceae Turicibacter, is indicative of non-CDI causative diarrhea associated with IBS.
Aspect 48) The method of aspect 46, wherein a decrease in relative abundance of at least one of: Collinsella aerofaciens; Veillonellaceae Veillonella; Lachnospiraceae Lachnoclostridium; Blautia hominis; Gammaproteobacteria Enterobacterales; Bifidobacteriales Bifidobacteriaceae; Ruminococcaceae Intestinimonas; Faecalimonas umbilicata; Actinomycetales Actinomycetaceae; Actinobacteria Actinobacteria; Dorea formicigenerans; and Bacteria Proteobacteria, is indicative of non-CDI causative diarrhea associated with IBS.
Aspect 49) The method of aspect 46-48, wherein a change in relative abundance of at least four taxonomical features is indicative of non-CDI causative diarrhea associated with IBS.
Aspect 50) The method of aspect 33, wherein the characterization of the subject microbiome to determine whether the non-CDI causative diarrhea is associated with IBD UC or IBD CD comprises measuring one or more taxonomical features from a biological sample from the subject and determining changes in relative abundance, compared to a reference healthy gut microbiome, of at least one of: Lachnospiraceae Lachnoclostridium; Ruminococcaceae Intestinimonas; Acidaminococcaceae Acidaminococcus; Bacilli Lactobacillus; Actinomycetales Actinomycetaceae; Ruminococcaceae Subdoligranulum; Peptostreptococcaceae Romboutsia; Bifidobacterium boum; Bacillales Gemella; Peptostreptococcaceae Peptostreptococcus; Peptoniphilaceae Peptoniphilus; Clostridiales Incertae Sedis XI Parvimonas; Peptostreptococcaceae Terrisporobacter; Erysipelotrichaceae Faecalicoccus; Solobacterium moorei; Bacteroides xylanisolvens; Enterobacterales Enterobacteriaceae; Bacteroides koreensis; Bacteroides cellulosilyticus; Phascolarctobacterium faecium; and Bacteroides thetaiotaomicron, wherein the change in taxonomical feature relative abundance is indicative of non-CDI causative diarrhea associated with IBD UC.
Aspect 51) The method of aspect 50, wherein an increase in relative abundance of at least one of: Lachnospiraceae Lachnoclostridium; Ruminococcaceae Intestinimonas; Acidaminococcaceae Acidaminococcus; Bacilli Lactobacillus; Actinomycetales Actinomycetaceae; Ruminococcaceae Subdoligranulum; Peptostreptococcaceae Romboutsia; Bifidobacterium boum; Bacillales Gemella; Peptostreptococcaceae Peptostreptococcus; Peptoniphilaceae Peptoniphilus; Clostridiales Incertae Sedis XI Parvimonas; Peptostreptococcaceae Terrisporobacter; Erysipelotrichaceae Faecalicoccus; and Solobacterium moorei, is indicative of non-CDI causative diarrhea associated with IBD UC.
Aspect 52) The method of aspect 50, wherein a decrease in relative abundance of at least one of: Bacteroides xylanisolvens; Enterobacterales Enterobacteriaceae; Bacteroides koreensis; Bacteroides cellulosilyticus; Phascolarctobacterium faecium; and Bacteroides thetaiotaomicron, is indicative of non-CDI causative diarrhea associated with IBD UC.
Aspect 53) The method of aspect 50-52, wherein a change in relative abundance of at least four taxonomical features is indicative of non-CDI causative diarrhea associated with IBD UC.
Aspect 54) The method of aspect 33, wherein the characterization of the subject microbiome to determine whether the non-CDI causative diarrhea is associated with IBD UC or IBD CD comprises measuring one or more taxonomical features from a biological sample from the subject and determining changes in relative abundance, compared to a reference healthy gut microbiome, of at least one of: Faecalimonas umbilicata; Lachnospiraceae Lachnoclostridium; Lachnoclostridium [Clostridium] bolteae; Proteobacteria Gammaproteobacteria; Bacilli Lactobacillus; Actinomycetales Actinomycetaceae; Gammaproteobacteria Enterobacterales; Rosenbergiella collisarenosi; Rosenbergiella nectarea; Veillonellaceae Veillonella; Acidaminococcaceae Acidaminococcus; Blautia hominis; Bifidobacterium boum; Enterobacteriaceae Cedecea; Ruminococcaceae Intestinimonas; Peptostreptococcaceae Romboutsia; Fusobacteriaceae Fusobacterium; Fusobacterium nucleatum; Megasphaera micronuciformis; Romboutsia timonensis; Faecalibacterium prausnitzii; Coprococcus catus; Alistipes shahii; Bacteroides cellulosilyticus; Bacteroides thetaiotaomicron; Coprococcus eutactus; Turicibacter sanguinis; Colidextribacter massiliensis; and Methanobrevibacter smithii; wherein the change in taxonomical feature relative abundance is indicative of non-CDI causative diarrhea associated with IBD CD.
Aspect 55) The method of aspect 54, wherein an increase in relative abundance of at least one of: Faecalimonas umbilicata; Lachnospiraceae Lachnoclostridium; Lachnoclostridium [Clostridium] bolteae; Proteobacteria Gammaproteobacteria; Bacilli Lactobacillus; Actinomycetales Actinomycetaceae; Gammaproteobacteria Enterobacterales; Rosenbergiella collisarenosi; Rosenbergiella nectarea; Veillonellaceae Veillonella; Acidaminococcaceae Acidaminococcus; Blautia hominis; Bifidobacterium boum; Enterobacteriaceae Cedecea; Ruminococcaceae Intestinimonas; Peptostreptococcaceae Romboutsia; Fusobacteriaceae Fusobacterium; Fusobacterium nucleatum; and Megasphaera micronuciformis, is indicative of non-CDI causative diarrhea associated with IBD CD.
Aspect 56) The method of aspect 54, wherein a decrease in relative abundance of at least one of: Romboutsia timonensis; Faecalibacterium prausnitzii; Coprococcus catus; Alistipes shahii; Bacteroides cellulosilyticus; Bacteroides thetaiotaomicron; Coprococcus eutactus; Turicibacter sanguinis; Colidextribacter massiliensis; and Methanobrevibacter smithii, is indicative of non-CDI causative diarrhea associated with IBD CD.
Aspect 57) The method of aspect 54-56, wherein a change in relative abundance of at least four taxonomical features is indicative of non-CDI causative diarrhea associated with IBD CD.
Aspect 58) The method of aspect 33, wherein the characterization of the subject microbiome to determine whether the non-CDI causative diarrhea is associated with IBD UC or IBD CD comprises measuring one or more taxonomical features from a biological sample from the subject and determining changes in relative abundance, compared to a reference dysbiosis IBD UC gut microbiome, of at least one of: Faecalimonas umbilicata; Blautia [Ruminococcus] gnavus; Lachnoclostridium [Clostridium] boltede; Erysipelatoclostridium ramosum; Veillonellaceae Veillonella; Enterobacterales Enterobacteriaceae; Blautia caecimuris; Fusobacteriaceae Fusobacterium; Fusobacterium nucleatum; Faecalibacterium prausnitzii; Ruminococcaceae Oscillibacter; Ruminococcaceae Clostridium IV; Romboutsia timonensis; Ruminococcaceae Pseudoflavonifractor; Erysipelotrichales Erysipelotrichaceae; and Methanobacteriaceae Methanobrevibacter; wherein the change in taxonomical feature relative abundance is indicative of non-CDI causative diarrhea associated with IBD CD.
Aspect 59) The method of aspect 58, wherein an increase in relative abundance of at least one of: Faecalimonas umbilicata; Blautia [Ruminococcus] gnavus; Lachnoclostridium [Clostridium] bolteae; Erysipelatoclostridium ramosum; Veillonellaceae Veillonella; Enterobacterales Enterobacteriaceae; Blautia caecimuris; Fusobacteriaceae Fusobacterium; and Fusobacterium nucleatum, is indicative of non-CDI causative diarrhea associated with IBD CD.
Aspect 60) The method of aspect 58, wherein a decrease in relative abundance of at least one of: Faecalibacterium prausnitzii; Ruminococcaceae Oscillibacter; Ruminococcaceae Clostridium IV; Romboutsia timonensis; Ruminococcaceae Pseudoflavonifractor; Erysipelotrichales Erysipelotrichaceae; and Methanobacteriaceae Methanobrevibacter, is indicative of non-CDI causative diarrhea associated with IBD CD.
Aspect 61) The method of aspect 58-60, wherein a change in relative abundance of at least four taxonomical features is indicative of non-CDI causative diarrhea associated with IBD CD.
Aspect 62) The method of aspect 13, wherein the measuring of one or more taxonomical features from a biological sample from the individual comprises determining changes in relative abundance, compared to a reference dysbiosis gut microbiome from individuals with CDI, of at least one of: Anaerostipes hadrus; Faecalibacterium prausnitzii; Lachnospiraceae Coprococcus; Lachnospiraceae [Eubacterium] rectale; Lachnospiraceae Roseburia; Lachnospiraceae Fusicatenibacter; Lachnospiraceae Dorea; Ruminococcaceae Ruminococcus; Roseburia inulinivorans; Dorea longicatena; Roseburia intestinalis; Lachnospiraceae Anaerostipes; Coprococcus comes; Lactobacillus rogosae; Romboutsia timonensis; Saccharibacteria Incertae Sedis; Eubacterium [Eubacterium] eligens; Bacteroides plebeius; Lachnospiraceae Blautia; Roseburia faecis; Blautia obeum; Coprococcus catus; Betaproteobacteria Burkholderiales; Prevotella copri; Burkholderiales Burkholderiaceae; Caulobacter segnis; Caulobacterales Caulobacteraceae; Burkholderiaceae Burkholderia; Burkholderia ambifaria; Prevotellaceae Prevotella; Burkholderiales Comamonadaceae; Burkholderia thailandensis; Alistipes obesi; Blautia stercoris; Ruminococcus bromii; Pelomonas aquatica; Peptostreptococcaceae Romboutsia; Bacteroides coprocola; Streptococcus thermophilus; Ruminococcaceae Oscillibacter; Alistipes ihumii; Ruminococcus callidus; Phyllobacteriaceae Phyllobacterium; Coprococcus eutactus; Peptostreptococcaceae Intestinibacter; Clostridioides difficile; Enterobacterales Enterobacteriaceae; Enterococcaceae Enterococcus; Peptostreptococcaceae Clostridium XI; Gammaproteobacteria Enterobacterales; Bacilli Lactobacillus; Eggerthella lenta; Erysipelatoclostridium [Clostridium] innocuum; Enterobacteriaceae Citrobacter; Lachnospiraceae Clostridium XIVa; Lachnoclostridium [Clostridium] bolteae; Erysipelatoclostridium ramosum; Hungatella effluvia; Veillonella parvula; Lactobacilluseae Lactobacillus; Blautia [Ruminococcus] gnavus; Enterococcus saccharolyticus; and Coriobacteriaceae Eggerthella; wherein the change in taxonomical feature relative abundance is indicative of non-CDI causative diarrhea associated with IBS.
Aspect 63) The method of aspect 62, wherein an increase in relative abundance of at least one of: Anaerostipes hadrus; Faecalibacterium prausnitzii; Lachnospiraceae Coprococcus; Lachnospiraceae [Eubacterium] rectale; Lachnospiraceae Roseburia; Lachnospiraceae Fusicatenibacter; Lachnospiraceae Dorea; Ruminococcaceae Ruminococcus; Roseburia inulinivorans; Dorea longicatena; Roseburia intestinalis; Lachnospiraceae Anaerostipes; Coprococcus comes; Lactobacillus rogosae; Romboutsia timonensis; Saccharibacteria Incertae Sedis; Eubacterium [Eubacterium] eligens; Bacteroides plebeius; Lachnospiraceae Blautia; Roseburia faecis; Blautia obeum; Coprococcus catus; Betaproteobacteria Burkholderiales; Prevotella copri; Burkholderiales Burkholderiaceae; Caulobacter segnis; Caulobacterales Caulobacteraceae; Burkholderiaceae Burkholderia; Burkholderia ambifaria; Prevotellaceae Prevotella; Burkholderiales Comamonadaceae; Burkholderia thailandensis; Alistipes obesi; Blautia stercoris; Ruminococcus bromii; Pelomonas aquatica; Peptostreptococcaceae Romboutsia; Bacteroides coprocola; Streptococcus thermophilus; Ruminococcaceae Oscillibacter; Alistipes ihumii; Ruminococcus callidus; Phyllobacteriaceae Phyllobacterium; Coprococcus eutactus; and Peptostreptococcaceae Intestinibacter, is indicative of non-CDI causative diarrhea associated with IBS.
Aspect 64) The method of aspect 62, wherein a decrease in relative abundance of at least one of: Clostridioides difficile; Enterobacterales Enterobacteriaceae; Enterobacteriales Enterobacteriaceae; Enterococcaceae Enterococcus; Peptostreptococcaceae Clostridium XI; Gammaproteobacteria Enterobacterales; Bacilli Lactobacilluses; Eggerthella lenta; Erysipelatoclostridium [Clostridium] innocuum; Enterobacteriaceae Citrobacter; Lachnospiraceae Clostridium XIVa; Lachnoclostridium [Clostridium] bolteae; Erysipelatoclostridium ramosum; Hungatella effluvia; Veillonella parvula; Lactobacilluseae Lactobacillus; Blautia [Ruminococcus] gnavus; Enterococcus saccharolyticus; and Coriobacteriaceae Eggerthella, is indicative of non-CDI causative diarrhea associated with IBS.
Aspect 65) The method of aspect 62-64, wherein a change in relative abundance of at least four taxonomical features is indicative of non-CDI causative diarrhea associated with IBS.
Aspect 66) The method of aspect 13, wherein the measuring of one or more taxonomical features from a biological sample from the individual comprises determining changes in relative abundance, compared to a reference dysbiosis gut microbiome from individuals with CDI, of at least one of: Anaerostipes hadrus; Lachnospiraceae Dorea; Dorea longicatena; Lachnospiraceae Coprococcus; Lachnospiraceae Roseburia; Blautia obeum; Coprococcus comes; Roseburia intestinalis; Lactobacillus rogosae; Eubacterium [Eubacterium] eligens; Bacteroidia Bacteroidales; Peptostreptococcaceae Romboutsia; Coriobacteriaceae Collinsella; Acidaminococcaceae Acidaminococcus; Prevotellaceae Prevotella; Prevotella copri; Coriobacteriales Coriobacteriaceae; Ruminococcaceae Ruminococcus; Alistipes obesi; Betaproteobacteria Burkholderiales; Ruminococcus bromii; Lachnospiraceae Lachnoclostridium; Bifidobacterium adolescentis; Actinomycetales Actinomycetaceae; Ruminococcaceae Intestinimonas; Bacteroides eggerthii; Clostridioides difficile; Peptostreptococcaceae Clostridium XI; Enterobacteriaceae Citrobacter; Enterobacterales Enterobacteriaceae; Veillonella parvula; Enterococcaceae Enterococcus; Lactobacillus Enterococcaceae; Erysipelatoclostridium [Clostridium] innocuum; Pectobacterium carotovorum; Enterobacteriales Enterobacteriaceae; Bacteroides xylanisolvens; Enterococcus saccharolyticus; Clostridium paraputrificum; Salmonella enterica; Erysipelatoclostridium ramosum; Hungatella effluvia; Bacteroides koreensis; Bacilli Lactobacillus; Blautia product; Klebsiella quasipneumoniae; Ruthenibacterium lactatiformans; Veillonella dispar; Coriobacteriaceae Eggerthella; and Lachnospiraceae Hungatella; wherein the change in taxonomical feature relative abundance is indicative of non-CDI causative diarrhea associated with IBD.
Aspect 67) The method of aspect 66, wherein an increase in relative abundance of at least one of: Anaerostipes hadrus; Lachnospiraceae Dorea; Dorea longicatena; Lachnospiraceae Coprococcus; Lachnospiraceae Roseburia; Blautia obeum; Coprococcus comes; Roseburia intestinalis; Lactobacillus rogosae; Eubacterium [Eubacterium] eligens; Bacteroidia Bacteroidales; Peptostreptococcaceae Romboutsia; Coriobacteriaceae Collinsella; Acidaminococcaceae Acidaminococcus; Prevotellaceae Prevotella; Prevotella copri; Coriobacteriales Coriobacteriaceae; Ruminococcaceae Ruminococcus; Alistipes obesi; Betaproteobacteria Burkholderiales; Ruminococcus bromii; Lachnospiraceae Lachnoclostridium; Bifidobacterium adolescentis; Actinomycetales Actinomycetaceae; Ruminococcaceae Intestinimonas; and Bacteroides eggerthii, is indicative of non-CDI causative diarrhea associated with IBD.
Aspect 68) The method of aspect 66, wherein a decrease in relative abundance of at least one of: Clostridioides difficile; Peptostreptococcaceae Clostridium XI; Enterobacteriaceae Citrobacter; Enterobacterales Enterobacteriaceae; Veillonella parvula; Enterococcaceae Enterococcus; Lactobacillus Enterococcacede; Erysipelatoclostridium [Clostridium] innocuum; Pectobacterium carotovorum; Enterobacteriales Enterobacteriaceae; Bacteroides xylanisolvens; Enterococcus saccharolyticus; Clostridium paraputrificum; Salmonella enterica; Erysipelatoclostridium ramosum; Hungatella effluvia; Bacteroides koreensis; Bacilli Lactobacillus; Blautia product; Klebsiella quasipneumoniae; Ruthenibacterium lactatiformans; Veillonella dispar; Coriobacteriaceae Eggerthella; and Lachnospiraceae Hungatella, is indicative of non-CDI causative diarrhea associated with IBD.
Aspect 69) The method of aspect 66-68, wherein a change in relative abundance of at least four taxonomical features is indicative of non-CDI causative diarrhea associated with IBD.
Aspect 70) A method of treating an individual having diarrhea comprising: measuring for one or more metabolic features from a biological sample from the individual; and reducing the administration of antibiotics and/or antimicrobial treatment to the individual when the individual has presence or absence or a certain level of one or more feature(s) indicative of non-CDI causative diarrhea; or administering antibiotics and/or antimicrobial treatment to the individual when the individual has presence or absence or a certain level of one or more feature(s) indicative of pathogenic infection.
Aspect 71) The method of aspect 70, wherein the antibiotics and/or antimicrobial treatment comprise at least one of the antibiotics selected from the group consisting of a small molecule antibiotic, an antibiotic derived from a natural product, a microbial composition, an antibody suitable for neutralizing pathogenic infections, a therapeutic, contact isolation, and any combination thereof.
Aspect 72) The method of aspect 71, with the proviso that if the non-CDI causative diarrhea is irritable bowel syndrome (IBS), administration of the antibiotic and/or antimicrobial rifaximin is not reduced.
Aspect 73) The method of aspect 71, wherein the antibiotics and/or antimicrobial treatment comprises at least one of vancomycin, fidaxomicin, and bezlotoxumab.
Aspect 74) The method of aspect 73, wherein the treatment is vancomycin, and optionally the treatment dosage is at least 125 mg four times per day for 10 days, wherein the treatment is fidaxomicin, and optionally the treatment dosage is at least 200 mg twice daily for 10 days, and/or wherein the treatment is bezlotoxumab.
Aspect 75) The method of any one of aspects 70-74, wherein the pathogenic diarrhea classification or non-CDI causative diarrhea classification is characterized by measuring the presence, absence, and/or relative quantity of at least 10 metabolic features described in any one of Tables 1-8.
Aspect 76) The method aspect 75, wherein the pathogenic diarrhea classification or non-CDI causative diarrhea classification is characterized by measuring the presence, absence, and/or relative quantity of at least 20 metabolic features described in any one of Tables 1-8.
Aspect 77) The method of aspect 76, wherein the pathogenic diarrhea classification or non-CDI causative diarrhea classification is characterized by measuring the presence, absence, and/or relative quantity of at least 40 metabolic features described in any one of Tables 1-8.
Aspect 78) The method of aspect 76, wherein the pathogenic diarrhea classification or non-CDI causative diarrhea classification is characterized by measuring the presence, absence, and/or relative quantity of at least 60 metabolic features described in any one of Tables 1-8.
Aspect 79) The method of aspect 76, wherein the pathogenic diarrhea classification or non-CDI causative diarrhea classification is characterized by measuring the presence, absence, and/or relative quantity of at least 80 metabolic features described in any one of Tables 1-8.
Aspect 80) The method of aspect 76, wherein the pathogenic diarrhea classification or non-CDI causative diarrhea classification is characterized by measuring the presence, absence, and/or relative quantity of at least 100 metabolic features described in any one Tables 1-8.
Aspect 81) The method of any one of aspects 76-80, wherein the pathogenic diarrhea classification or non-CDI causative diarrhea classification comprises characterization using more than one of Tables 1-8.
Aspect 82) The method of aspect 81, wherein the more than one characterization using Tables 1-8 is sequential.
Aspect 83) The method of aspect 81 or 82, wherein the more than one characterization using Tables 1-8 comprises first characterizing using Tables 3 and/or 7, followed by characterization using one or more of the remaining Tables.
Aspect 84) The method of any one of aspects 70-83, wherein the measuring of one or more metabolic features comprises at least one of analyzing one or more nucleic acids in the sample, analyzing one or more metabolites in the sample, and analyzing one or more proteins in the sample.
Aspect 85) The method of aspect 84, wherein the nucleic acid is analyzed by sequencing, polymerase chain reaction, isothermal amplification, bioinformatics, or any combination thereof.
Aspect 86) The method of aspect 85, wherein the nucleic acid analyzed is 16S ribosomal RNA.
Aspect 87) The method of aspect 84, wherein the metabolites are analyzed by mass spectrometry, ELISA, chromatography, or any combination thereof.
Aspect 88) The method of aspect 84, wherein the proteins are analyzed by mass spectrometry, ELISA, chromatography. Western blotting, immunoprecipitation, immunoelectrophoresis, or any combination thereof.
Aspect 89) The method of any one of aspects 70-88, wherein when reducing the administration of antibiotics and/or antimicrobial treatment to the individual when the individual has presence or absence or a certain level of one or more feature(s) indicative of non-CDI causative diarrhea, the subject microbiome is further characterized to determine whether the non-CDI causative diarrhea is associated with irritable bowel syndrome (IBS) or inflammatory bowel disease (IBD), and treatment is modified accordingly.
Aspect 90) The method of aspect 89, wherein the non-CDI causative diarrhea is associated with IBD, the IBD is further characterized to determine whether the IBD is Ulcerative Colitis (UC) or Crohn's Disease (CD), and treatment is modified accordingly.
Aspect 91) The method of aspect 70, wherein the measuring of one or more metabolic features from a biological sample from the individual comprises determining changes in relative abundance, compared to a reference healthy gut microbiome, of at least one of: PWY-7431 (aromatic biogenic amine degradation (bacteria)); PWY-7159 (chlorophyllide a biosynthesis III (acrobic, light independent)); PWY-5531 (chlorophyllide a biosynthesis II (anaerobic)); CHLOROPHYLL-SYN (chlorophyllide a biosynthesis I (aerobic, light-dependent)); P562-PWY (myo-inositol degradation I); PWY-3781 (aerobic respiration I (cytochrome c)); TYRFUMCAT-PWY (L-tyrosine degradation I); HCAMHPDEG-PWY (3-phenylpropanoate and 3-(3-hydroxyphenyl) propanoate degradation to 2-oxopent-4-enoate); PWY-6071 (superpathway of phenylethylamine degradation); and PWY-6690 (cinnamate and 3-hydroxycinnamate degradation to 2-oxopent-4-enoate); wherein the change in metabolic feature relative abundance is indicative of non-CDI causative diarrhea associated with IBS.
Aspect 92) The method of aspect 91, wherein an increase in relative abundance of at least one of: PWY-7431 (aromatic biogenic amine degradation (bacteria)); PWY-7159 (chlorophyllide a biosynthesis III (aerobic, light independent)); PWY-5531 (chlorophyllide a biosynthesis II (anaerobic)); CHLOROPHYLL-SYN (chlorophyllide a biosynthesis I (aerobic, light-dependent)); P562-PWY (myo-inositol degradation I); PWY-3781 (aerobic respiration I (cytochrome c)); and TYRFUMCAT-PWY (L-tyrosine degradation I), is indicative of non-CDI causative diarrhea associated with IBS.
Aspect 93) The method of aspect 91, wherein a decrease in relative abundance of at least one of: HCAMHPDEG-PWY (3-phenylpropanoate and 3-(3-hydroxyphenyl) propanoate degradation to 2-oxopent-4-enoate); PWY-6071 (superpathway of phenylethylamine degradation); and PWY-6690 (cinnamate and 3-hydroxycinnamate degradation to 2-oxopent-4-enoate), is indicative of non-CDI causative diarrhea associated with IBS.
Aspect 94) The method of aspect 91-93, wherein a change in relative abundance of at least three metabolic features is indicative of non-CDI causative diarrhea associated with IBS.
Aspect 95) The method of aspect 70, wherein the measuring of one or more metabolic features from a biological sample from the individual comprises determining changes in relative abundance, compared to a reference healthy gut microbiome, of at least one of: ASPASN-PWY (superpathway of L-aspartate and L-asparagine biosynthesis); DHGLUCONATE-PYR-CAT-PWY (glucose degradation (oxidative)); DTDPRHAMSYN-PWY (dTDP-L-rhamnose biosynthesis I); FASYN-ELONG-PWY (fatty acid elongation-saturated); GALACTARDEG-PWY (D-galactarate degradation I); GLUCARDEG-PWY (D-glucarate degradation I); GLUCARGALACTSUPER-PWY (superpathway of D-glucarate and D-galactarate degradation); GLUCONEO-PWY (gluconcogenesis I); GLUCUROCAT-PWY (superpathway of & beta; -D-glucuronide and D-glucuronate degradation); GLYCOLYSIS (glycolysis I (from glucose 6-phosphate)); GLYCOLYSIS-E-D (superpathway of glycolysis and Entner-Doudoroff); HEMESYN2-PWY (heme biosynthesis II (anacrobic)); HISDEG-PWY (L-histidine degradation I); METHGLYUT-PWY (superpathway of methylglyoxal degradation); P125-PWY (superpathway of (R,R)-butanediol biosynthesis); P162-PWY (L-glutamate degradation V (via hydroxyglutarate)); P163-PWY (L-lysine fermentation to acetate and butanoate); P42-PWY (incomplete reductive TCA cycle); PENTOSE-P-PWY (pentose phosphate pathway); POLYISOPRENSYN-PWY (polyisoprenoid biosynthesis (E. coli)); PWY0-1241 (ADP-L-glycero-& beta; -D-manno-heptose biosynthesis); PWY0-1533 (methylphosphonate degradation I); PWY0-41 (allantoin degradation IV (anaerobic)); PWY0-845 (superpathway of pyridoxal 5′-phosphate biosynthesis and salvage); PWY-5028 (L-histidine degradation II); PWY-5484 (glycolysis II (from fructose 6-phosphate)); PWY-5659 (GDP-mannose biosynthesis); PWY-5695 (urate biosynthesis/inosine 5′-phosphate degradation); PWY-5705 (allantoin degradation to glyoxylate III); PWY-5913 (TCA cycle VI (obligate autotrophs)); PWY-5971 (palmitate biosynthesis II (bacteria and plants)); PWY-6125 (superpathway of guanosine nucleotides de novo biosynthesis II); PWY-6353 (purine nucleotides degradation II (acrobic)); PWY-6396 (superpathway of 2,3-butanediol biosynthesis); PWY-6507 (4-deoxy-L-threo-hex-4-enopyranuronate degradation); PWY-6572 (chondroitin sulfate degradation I (bacterial)); PWY-6588 (pyruvate fermentation to acetone); PWY-6608 (guanosine nucleotides degradation III); PWY-6703 (preQ0 biosynthesis); PWY-6891 (thiazole biosynthesis II (Bacillus)); PWY-6895 (superpathway of thiamin diphosphate biosynthesis II); PWY-6901 (superpathway of glucose and xylose degradation); PWY-6969 (TCA cycle V (2-oxoglutarate: ferredoxin oxidoreductase)); PWY-7013 (L-1,2-propanediol degradation); PWY-7199 (pyrimidine deoxyribonucleosides salvage); PWY-7220 (adenosine deoxyribonucleotides de novo biosynthesis II); PWY-7222 (guanosine deoxyribonucleotides de novo biosynthesis II); PWY-7237 (myo-, chiro- and scillo-inositol degradation); PWY-7242 (D-fructuronate degradation); PWY-7328 (superpathway of UDP-glucose-derived O-antigen building blocks biosynthesis); PWY-7456 (mannan degradation); PYRIDOXSYN-PWY (pyridoxal 5′-phosphate biosynthesis I); SALVADEHYPOX-PWY (adenosine nucleotides degradation II); TCA (TCA cycle I (prokaryotic)); THISYN-PWY (superpathway of thiamin diphosphate biosynthesis I); and VALDEG-PWY (L-valine degradation I); wherein the change in metabolic feature relative abundance is indicative of non-CDI causative diarrhea associated with IBD.
Aspect 96) The method of aspect 95, wherein an increase in relative abundance of at least one of: ASPASN-PWY (superpathway of L-aspartate and L-asparagine biosynthesis); DHGLUCONATE-PYR-CAT-PWY (glucose degradation (oxidative)); DTDPRHAMSYN-PWY (dTDP-L-rhamnose biosynthesis I); FASYN-ELONG-PWY (fatty acid elongation-saturated); GALACTARDEG-PWY (D-galactarate degradation I); GLUCARDEG-PWY (D-glucarate degradation I); GLUCARGALACTSUPER-PWY (superpathway of D-glucarate and D-galactarate degradation); GLUCONEO-PWY (gluconeogenesis I); GLUCUROCAT-PWY (superpathway of & beta; -D-glucuronide and D-glucuronate degradation); GLYCOLYSIS (glycolysis I (from glucose 6-phosphate)); GLYCOLYSIS-E-D (superpathway of glycolysis and Entner-Doudoroff); HEMESYN2-PWY (heme biosynthesis II (anaerobic)); HISDEG-PWY (L-histidine degradation I); METHGLYUT-PWY (superpathway of methylglyoxal degradation); P125-PWY (superpathway of (R,R)-butanediol biosynthesis); P162-PWY (L-glutamate degradation V (via hydroxyglutarate)); P163-PWY (L-lysine fermentation to acetate and butanoate); P42-PWY (incomplete reductive TCA cycle); PENTOSE-P-PWY (pentose phosphate pathway); POLYISOPRENSYN-PWY (polyisoprenoid biosynthesis (E. coli)); PWY0-1241 (ADP-L-glycero-& beta; -D-manno-heptose biosynthesis); PWY0-1533 (methylphosphonate degradation I); PWY0-41 (allantoin degradation IV (anaerobic)); PWY0-845 (superpathway of pyridoxal 5′-phosphate biosynthesis and salvage); PWY-5028 (L-histidine degradation II); PWY-5484 (glycolysis II (from fructose 6-phosphate)); PWY-5659 (GDP-mannose biosynthesis); PWY-5695 (urate biosynthesis/inosine 5′-phosphate degradation); PWY-5705 (allantoin degradation to glyoxylate III); PWY-5913 (TCA cycle VI (obligate autotrophs)); PWY-5971 (palmitate biosynthesis II (bacteria and plants)); PWY-6125 (superpathway of guanosine nucleotides de novo biosynthesis II); PWY-6353 (purine nucleotides degradation II (aerobic)); PWY-6396 (superpathway of 2,3-butanediol biosynthesis); PWY-6507 (4-deoxy-L-threo-hex-4-enopyranuronate degradation); PWY-6572 (chondroitin sulfate degradation I (bacterial)); PWY-6588 (pyruvate fermentation to acetone); PWY-6608 (guanosine nucleotides degradation III); PWY-6703 (preQ0 biosynthesis); PWY-6891 (thiazole biosynthesis II (Bacillus)); PWY-6895 (superpathway of thiamin diphosphate biosynthesis II); PWY-6901 (superpathway of glucose and xylose degradation); PWY-6969 (TCA cycle V (2-oxoglutarate: ferredoxin oxidoreductase)); PWY-7013 (L-1,2-propanediol degradation); PWY-7199 (pyrimidine deoxyribonucleosides salvage); PWY-7220 (adenosine deoxyribonucleotides de novo biosynthesis II); PWY-7222 (guanosine deoxyribonucleotides de novo biosynthesis II); PWY-7237 (myo-, chiro- and scillo-inositol degradation); PWY-7242 (D-fructuronate degradation); PWY-7328 (superpathway of UDP-glucose-derived O-antigen building blocks biosynthesis); PWY-7456 (mannan degradation); PYRIDOXSYN-PWY (pyridoxal 5′-phosphate biosynthesis I); SALVADEHYPOX-PWY (adenosine nucleotides degradation II); TCA (TCA cycle I (prokaryotic)); THISYN-PWY (superpathway of thiamin diphosphate biosynthesis I); and VALDEG-PWY (L-valine degradation I), is indicative of non-CDI causative diarrhea associated with IBD.
Aspect 97) The method of any one of aspect 95-96, wherein a change in relative abundance of at least four metabolic features is indicative of non-CDI causative diarrhea associated with IBD.
Aspect 98) The method of aspect 89, wherein the characterization of the subject microbiome to determine whether the non-CDI causative diarrhea is associated with IBS or IBD comprises measuring one or more metabolic features from a biological sample from the subject and determining changes in relative abundance, compared to a reference dysbiosis IBS gut microbiome, of at least one of: ASPASN-PWY (superpathway of L-aspartate and L-asparagine biosynthesis); GALACT-GLUCUROCAT-PWY (superpathway of hexuronide and hexuronate degradation); ANAEROFRUCAT-PWY (homolactic fermentation); DHGLUCONATE-PYR-CAT-PWY (glucose degradation (oxidative)); GLUCUROCAT-PWY (superpathway of & beta; -D-glucuronide and D-glucuronate degradation); PWY0-781 (aspartate superpathway); PWY-7242 (D-fructuronate degradation); GOLPDLCAT-PWY (superpathway of glycerol degradation to 1,3-propanediol); PWY-7013 (L-1,2-propanediol degradation); P124-PWY (Bifidobacterium shunt); P122-PWY (heterolactic fermentation); REDCITCYC (TCA cycle VIII (helicobacter)); DENOVOPURINE2-PWY (superpathway of purine nucleotides de novo biosynthesis II); GLUCARGALACTSUPER-PWY (superpathway of D-glucarate and D-galactarate degradation); GALACTARDEG-PWY (D-galactarate degradation I); PWY-6891 (thiazole biosynthesis II (Bacillus)); FUC-RHAMCAT-PWY (superpathway of fucose and rhamnose degradation); PWY-6895 (superpathway of thiamin diphosphate biosynthesis II); PWY0-41 (allantoin degradation IV (anaerobic)); VALDEG-PWY (L-valine degradation I); PWY-7187 (pyrimidine deoxyribonucleotides de novo biosynthesis II); PWY0-1533 (methylphosphonate degradation I); GLUCARDEG-PWY (D-glucarate degradation I); TYRFUMCAT-PWY (L-tyrosine degradation I); PWY-7431 (aromatic biogenic amine degradation (bacteria)); PWY-7094 (fatty acid salvage); and LEU-DEG2-PWY (L-leucine degradation I); wherein the change in metabolic feature relative abundance is indicative of non-CDI causative diarrhea associated with IBD.
Aspect 99) The method of aspect 98, wherein an increase in relative abundance of at least one of: ASPASN-PWY (superpathway of L-aspartate and L-asparagine biosynthesis); GALACT-GLUCUROCAT-PWY (superpathway of hexuronide and hexuronate degradation); ANAEROFRUCAT-PWY (homolactic fermentation); DHGLUCONATE-PYR-CAT-PWY (glucose degradation (oxidative)); GLUCUROCAT-PWY (superpathway of & beta; -D-glucuronide and D-glucuronate degradation); PWY0-781 (aspartate superpathway); PWY-7242 (D-fructuronate degradation); GOLPDLCAT-PWY (superpathway of glycerol degradation to 1,3-propanediol); PWY-7013 (L-1,2-propanediol degradation); P124-PWY (Bifidobacterium shunt); P122-PWY (heterolactic fermentation); REDCITCYC (TCA cycle VIII (helicobacter)); DENOVOPURINE2-PWY (superpathway of purine nucleotides de novo biosynthesis II); GLUCARGALACTSUPER-PWY (superpathway of D-glucarate and D-galactarate degradation); GALACTARDEG-PWY (D-galactarate degradation I); PWY-6891 (thiazole biosynthesis II (Bacillus)); FUC-RHAMCAT-PWY (superpathway of fucose and rhamnose degradation); PWY-6895 (superpathway of thiamin diphosphate biosynthesis II); PWY0-41 (allantoin degradation IV (anacrobic)); VALDEG-PWY (L-valine degradation I); PWY-7187 (pyrimidine deoxyribonucleotides de novo biosynthesis II); PWY0-1533 (methylphosphonate degradation I); and GLUCARDEG-PWY (D-glucarate degradation I), is indicative of non-CDI causative diarrhea associated with IBD.
Aspect 100) The method of aspect 98, wherein a decrease in relative abundance of at least one of: TYRFUMCAT-PWY (L-tyrosine degradation I); PWY-7431 (aromatic biogenic amine degradation (bacteria)); PWY-7094 (fatty acid salvage); and LEU-DEG2-PWY (L-leucine degradation I), is indicative of non-CDI causative diarrhea associated with IBD.
Aspect 101) The method of aspect 98-100, wherein a change in relative abundance of at least four metabolic features is indicative of non-CDI causative diarrhea associated with IBD.
Aspect 102) The method of aspect 90, wherein the characterization of the subject microbiome to determine whether the non-CDI causative diarrhea is associated with IBD UC or IBD CD comprises measuring one or more metabolic features from a biological sample from the subject and determining changes in relative abundance, compared to a reference healthy gut microbiome, of at least one of: PWY-5484 (glycolysis II (from fructose 6-phosphate)); PWY-7220 (adenosine deoxyribonucleotides de novo biosynthesis II); DTDPRHAMSYN-PWY (dTDP-L-rhamnose biosynthesis I); GLUCONEO-PWY (gluconcogenesis I); PWY-7222 (guanosine deoxyribonucleotides de novo biosynthesis II); GLYCOLYSIS (glycolysis I (from glucose 6-phosphate)); POLYISOPRENSYN-PWY (polyisoprenoid biosynthesis (E. coli)); PWY-6969 (TCA cycle V (2-oxoglutarate: ferredoxin oxidoreductase)); PENTOSE-P-PWY (pentose phosphate pathway); PWY-5971 (palmitate biosynthesis II (bacteria and plants)); PWY-7199 (pyrimidine deoxyribonucleosides salvage); PWY-6901 (superpathway of glucose and xylose degradation); PWY-5659 (GDP-mannose biosynthesis); PWY-5695 (urate biosynthesis/inosine 5′-phosphate degradation); PWY-7456 (mannan degradation); VALDEG-PWY (L-valine degradation I); ASPASN-PWY (superpathway of L-aspartate and L-asparagine biosynthesis); TCA (TCA cycle I (prokaryotic)); FASYN-ELONG-PWY (fatty acid elongation-saturated); PWY-6703 (preQ0 biosynthesis); HEMESYN2-PWY (heme biosynthesis II (anaerobic)); P42-PWY (incomplete reductive TCA cycle); PWY-5913 (TCA cycle VI (obligate autotrophs)); P162-PWY (L-glutamate degradation V (via hydroxyglutarate)); PWY0-845 (superpathway of pyridoxal 5′-phosphate biosynthesis and salvage); P163-PWY (L-lysine fermentation to acetate and butanoate); THISYN-PWY (superpathway of thiamin diphosphate biosynthesis I); PWY-7242 (D-fructuronate degradation); PWY-6125 (superpathway of guanosine nucleotides de novo biosynthesis II); PWY-6572 (chondroitin sulfate degradation I (bacterial)); and PWY-6507 (4-deoxy-L-threo-hex-4-enopyranuronate degradation); wherein the change in metabolic feature relative abundance is indicative of non-CDI causative diarrhea associated with IBD UC.
Aspect 103) The method of aspect 102, wherein an increase in relative abundance of at least one of: PWY-5484 (glycolysis II (from fructose 6-phosphate)); PWY-7220 (adenosine deoxyribonucleotides de novo biosynthesis II); DTDPRHAMSYN-PWY (dTDP-L-rhamnose biosynthesis I); GLUCONEO-PWY (gluconcogenesis I); PWY-7222 (guanosine deoxyribonucleotides de novo biosynthesis II); GLYCOLYSIS (glycolysis I (from glucose 6-phosphate)); POLYISOPRENSYN-PWY (polyisoprenoid biosynthesis (E. coli)); PWY-6969 (TCA cycle V (2-oxoglutarate: ferredoxin oxidoreductase)); PENTOSE-P-PWY (pentose phosphate pathway); PWY-5971 (palmitate biosynthesis II (bacteria and plants)); PWY-7199 (pyrimidine deoxyribonucleosides salvage); PWY-6901 (superpathway of glucose and xylose degradation); PWY-5659 (GDP-mannose biosynthesis); PWY-5695 (urate biosynthesis/inosine 5′-phosphate degradation); PWY-7456 (mannan degradation); VALDEG-PWY (L-valine degradation I); ASPASN-PWY (superpathway of L-aspartate and L-asparagine biosynthesis); TCA (TCA cycle I (prokaryotic)); FASYN-ELONG-PWY (fatty acid elongation-saturated); PWY-6703 (preQ0 biosynthesis); HEMESYN2-PWY (heme biosynthesis II (anaerobic)); P42-PWY (incomplete reductive TCA cycle); PWY-5913 (TCA cycle VI (obligate autotrophs)); P162-PWY (L-glutamate degradation V (via hydroxyglutarate)); PWY0-845 (superpathway of pyridoxal 5′-phosphate biosynthesis and salvage); P163-PWY (L-lysine fermentation to acetate and butanoate); THISYN-PWY (superpathway of thiamin diphosphate biosynthesis I); PWY-7242 (D-fructuronate degradation); PWY-6125 (superpathway of guanosine nucleotides de novo biosynthesis II); PWY-6572 (chondroitin sulfate degradation I (bacterial)); and PWY-6507 (4-deoxy-L-threo-hex-4-enopyranuronate degradation), is indicative of non-CDI causative diarrhea associated with IBD UC.
Aspect 104) The method of any one of aspects 102-103, wherein a change in relative abundance of at least four metabolic features is indicative of non-CDI causative diarrhea associated with IBD UC.
Aspect 105) The method of aspect 90, wherein the characterization of the subject microbiome to determine whether the non-CDI causative diarrhea is associated with IBD UC or IBD CD comprises measuring one or more metabolic features from a biological sample from the subject and determining changes in relative abundance, compared to a reference healthy gut microbiome, of at least one of: HEMESYN2-PWY (heme biosynthesis II (anaerobic)); PWY-6608 (guanosine nucleotides degradation III); SALVADEHYPOX-PWY (adenosine nucleotides degradation II); GALACTARDEG-PWY (D-galactarate degradation I); PWY-7013 (L-1,2-propanediol degradation); GLUCARGALACTSUPER-PWY (superpathway of D-glucarate and D-galactarate degradation); PWY-6353 (purine nucleotides degradation II (acrobic)); P125-PWY (superpathway of (R,R)-butanediol biosynthesis); GLUCUROCAT-PWY (superpathway of & beta; -D-glucuronide and D-glucuronate degradation); GLUCARDEG-PWY (D-glucarate degradation I); PENTOSE-P-PWY (pentose phosphate pathway); PWY-6891 (thiazole biosynthesis II (Bacillus)); HISDEG-PWY (L-histidine degradation I); PWY0-845 (superpathway of pyridoxal 5′-phosphate biosynthesis and salvage); PWY-5028 (L-histidine degradation II); PWY-7328 (superpathway of UDP-glucose-derived O-antigen building blocks biosynthesis); PWY-7237 (myo-, chiro- and scillo-inositol degradation); P162-PWY (L-glutamate degradation V (via hydroxyglutarate)); PWY-6588 (pyruvate fermentation to acetone); PWY0-1533 (methylphosphonate degradation I); PWY-6396 (superpathway of 2,3-butanediol biosynthesis); PYRIDOXSYN-PWY (pyridoxal 5′-phosphate biosynthesis I); PWY-6895 (superpathway of thiamin diphosphate biosynthesis II); PWY0-1241 (ADP-L-glycero-& beta; -D-manno-heptose biosynthesis); PWY0-41 (allantoin degradation IV (anaerobic)); DHGLUCONATE-PYR-CAT-PWY (glucose degradation (oxidative)); PWY-5705 (allantoin degradation to glyoxylate III); GLYCOLYSIS-E-D (superpathway of glycolysis and Entner-Doudoroff); and METHGLYUT-PWY (superpathway of methylglyoxal degradation); wherein the change in metabolic feature relative abundance is indicative of non-CDI causative diarrhea associated with IBD CD.
Aspect 106) The method of aspect 105, wherein an increase in relative abundance of at least one of: HEMESYN2-PWY (heme biosynthesis II (anaerobic)); PWY-6608 (guanosine nucleotides degradation III); SALVADEHYPOX-PWY (adenosine nucleotides degradation II); GALACTARDEG-PWY (D-galactarate degradation I); PWY-7013 (L-1,2-propanediol degradation); GLUCARGALACTSUPER-PWY (superpathway of D-glucarate and D-galactarate degradation); PWY-6353 (purine nucleotides degradation II (aerobic)); P125-PWY (superpathway of (R,R)-butanediol biosynthesis); GLUCUROCAT-PWY (superpathway of & beta; -D-glucuronide and D-glucuronate degradation); GLUCARDEG-PWY (D-glucarate degradation I); PENTOSE-P-PWY (pentose phosphate pathway); PWY-6891 (thiazole biosynthesis II (Bacillus)); HISDEG-PWY (L-histidine degradation I); PWY0-845 (superpathway of pyridoxal 5′-phosphate biosynthesis and salvage); PWY-5028 (L-histidine degradation II); PWY-7328 (superpathway of UDP-glucose-derived O-antigen building blocks biosynthesis); PWY-7237 (myo-, chiro- and scillo-inositol degradation); P162-PWY (L-glutamate degradation V (via hydroxyglutarate)); PWY-6588 (pyruvate fermentation to acetone); PWY0-1533 (methylphosphonate degradation I); PWY-6396 (superpathway of 2,3-butanediol biosynthesis); PYRIDOXSYN-PWY (pyridoxal 5′-phosphate biosynthesis I); PWY-6895 (superpathway of thiamin diphosphate biosynthesis II); PWY0-1241 (ADP-L-glycero-& beta; -D-manno-heptose biosynthesis); PWY0-41 (allantoin degradation IV (anacrobic)); DHGLUCONATE-PYR-CAT-PWY (glucose degradation (oxidative)); PWY-5705 (allantoin degradation to glyoxylate III); GLYCOLYSIS-E-D (superpathway of glycolysis and Entner-Doudoroff); and METHGLYUT-PWY (superpathway of methylglyoxal degradation), is indicative of non-CDI causative diarrhea associated with IBD CD.
Aspect 107) The method of any one of aspects 105-106, wherein a change in relative abundance of at least four metabolic features is indicative of non-CDI causative diarrhea associated with IBD CD.
Aspect 108) The method of aspect 90, wherein the characterization of the subject microbiome to determine whether the non-CDI causative diarrhea is associated with IBD UC or IBD CD comprises measuring one or more metabolic features from a biological sample from the subject and determining changes in relative abundance, compared to a reference dysbiosis IBD UC gut microbiome, of at least one of: ECASYN-PWY (enterobacterial common antigen biosynthesis); PPGPPMET-PWY (ppGpp biosynthesis); ORNARGDEG-PWY (superpathway of L-arginine and L-ornithine degradation); AST-PWY (L-arginine degradation II (AST pathway)); ARGDEG-PWY (superpathway of L-arginine, putrescine, and 4-aminobutanoate degradation); ORNDEG-PWY (superpathway of ornithine degradation); PWY0-1338 (polymyxin resistance); PWY-5028 (L-histidine degradation II); AEROBACTINSYN-PWY (acrobactin biosynthesis); PWY-6071 (superpathway of phenylethylamine degradation); PWY0-321 (phenylacetate degradation I (aerobic)); PWY0-1533 (methylphosphonate degradation I); P241-PWY (coenzyme B biosynthesis); PWY-6148 (tetrahydromethanopterin biosynthesis); PWY-6349 (CDP-archacol biosynthesis); PWY-6654 (phosphopantothenate biosynthesis III); METHANOGENESIS-PWY (methanogenesis from H2 and CO2); PWY-7286 (7-(3-amino-3-carboxypropyl)-wyosine biosynthesis); PWY-5198 (factor 420 biosynthesis); PWY-6167 (flavin biosynthesis II (archaca)); PWY-6641 (superpathway of sulfolactate degradation); PWY-6350 (archactidylinositol biosynthesis); PWY-6141 (archactidylserine and archaetidylethanolamine biosynthesis); PWY-6174 (mevalonate pathway II (archaca)); P261-PWY (coenzyme M biosynthesis I); and PWY-7391 (isoprene biosynthesis II (engineered)); wherein the change in metabolic feature relative abundance is indicative of non-CDI causative diarrhea associated with IBD CD.
Aspect 109) The method of aspect 108, wherein an increase in relative abundance of at least one of: ECASYN-PWY (enterobacterial common antigen biosynthesis); PPGPPMET-PWY (ppGpp biosynthesis); ORNARGDEG-PWY (superpathway of L-arginine and L-ornithine degradation); AST-PWY (L-arginine degradation II (AST pathway)); ARGDEG-PWY (superpathway of L-arginine, putrescine, and 4-aminobutanoate degradation); ORNDEG-PWY (superpathway of ornithinc degradation); PWY0-1338 (polymyxin resistance); PWY-5028 (L-histidine degradation II); AEROBACTINSYN-PWY (acrobactin biosynthesis); PWY-6071 (superpathway of phenylethylamine degradation); PWY0-321 (phenylacetate degradation I (acrobic)); and PWY0-1533 (methylphosphonate degradation I), is indicative of non-CDI causative diarrhea associated with IBD CD.
Aspect 110) The method of aspect 108, wherein a decrease in relative abundance of at least one of: P241-PWY (coenzyme B biosynthesis); PWY-6148 (tetrahydromethanopterin biosynthesis); PWY-6349 (CDP-archacol biosynthesis); PWY-6654 (phosphopantothenate biosynthesis III); METHANOGENESIS-PWY (methanogenesis from H2 and CO2); PWY-7286 (7-(3-amino-3-carboxypropyl)-wyosine biosynthesis); PWY-5198 (factor 420 biosynthesis); PWY-6167 (flavin biosynthesis II (archaca)); PWY-6641 (superpathway of sulfolactate degradation); PWY-6350 (archaetidylinositol biosynthesis); PWY-6141 (archaetidylserine and archactidylethanolamine biosynthesis); PWY-6174 (mevalonate pathway II (archaca)); P261-PWY (coenzyme M biosynthesis I); and PWY-7391 (isoprene biosynthesis II (engineered)), is indicative of non-CDI causative diarrhea associated with IBD CD.
Aspect 111) The method of any one of aspects 108-110, wherein a change in relative abundance of at least four metabolic features is indicative of non-CDI causative diarrhea associated with IBD CD.
Aspect 112) The method of aspect 70, wherein the measuring of one or more metabolic features from a biological sample from the individual comprises determining changes in relative abundance, compared to a reference dysbiosis gut microbiome from individuals with CDI, of at least one of: P221-PWY (octane oxidation); PWY-7374 (1,4-dihydroxy-6-naphthoate biosynthesis I); PWY-6263 (superpathway of menaquinol-8 biosynthesis II); CHLOROPHYLL-SYN (chlorophyllide a biosynthesis I (aerobic, light-dependent)); PWY-5198 (factor 420 biosynthesis); PWY-7286 (7-(3-amino-3-carboxypropyl)-wyosine biosynthesis); PWY-5531 (chlorophyllide a biosynthesis II (anaerobic)); PWY-6349 (CDP-archacol biosynthesis); PWY-5088 (L-glutamate degradation VIII (to propanoate)); PWY-6350 (archactidylinositol biosynthesis); PWY-7159 (chlorophyllide a biosynthesis III (aerobic, light independent)); PWY0-1533 (methylphosphonate degradation I); ORNDEG-PWY (superpathway of ornithine degradation); PWY0-1338 (polymyxin resistance); ORNARGDEG-PWY (superpathway of L-arginine and L-ornithine degradation); ECASYN-PWY (enterobacterial common antigen biosynthesis); ARGDEG-PWY (superpathway of L-arginine, putrescine, and 4-aminobutanoate degradation); GLYCOL-GLYOXDEG-PWY (superpathway of glycol metabolism and degradation); ENTBACSYN-PWY (enterobactin biosynthesis); THREOCAT-PWY (superpathway of L-threonine metabolism); GOLPDLCAT-PWY (superpathway of glycerol degradation to 1,3-propanediol); PWY-6071 (superpathway of phenylethylamine degradation); PWY0-1277 (3-phenylpropanoate and 3-(3-hydroxyphenyl) propanoate degradation); and PWY-6690 (cinnamate and 3-hydroxycinnamate degradation to 2-oxopent-4-enoate); wherein the change in metabolic feature relative abundance is indicative of non-CDI causative diarrhea associated with IBS.
Aspect 113) The method of aspect 112, wherein an increase in relative abundance of at least one of: P221-PWY (octane oxidation); PWY-7374 (1,4-dihydroxy-6-naphthoate biosynthesis I); PWY-6263 (superpathway of menaquinol-8 biosynthesis II); CHLOROPHYLL-SYN (chlorophyllide a biosynthesis I (aerobic, light-dependent)); PWY-5198 (factor 420 biosynthesis); PWY-7286 (7-(3-amino-3-carboxypropyl)-wyosine biosynthesis); PWY-5531 (chlorophyllide a biosynthesis II (anaerobic)); PWY-6349 (CDP-archacol biosynthesis); PWY-5088 (L-glutamate degradation VIII (to propanoate)); PWY-6350 (archactidylinositol biosynthesis); and PWY-7159 (chlorophyllide a biosynthesis III (aerobic, light independent)), is indicative of non-CDI causative diarrhea associated with IBS.
Aspect 114) The method of aspect 112, wherein a decrease in relative abundance of at least one of: PWY0-1533 (methylphosphonate degradation I); ORNDEG-PWY (superpathway of ornithinc degradation); PWY0-1338 (polymyxin resistance); ORNARGDEG-PWY (superpathway of L-arginine and L-ornithine degradation); ECASYN-PWY (enterobacterial common antigen biosynthesis); ARGDEG-PWY (superpathway of L-arginine, putrescine, and 4-aminobutanoate degradation); GLYCOL-GLYOXDEG-PWY (superpathway of glycol metabolism and degradation); ENTBACSYN-PWY (enterobactin biosynthesis); THREOCAT-PWY (superpathway of L-threonine metabolism); GOLPDLCAT-PWY (superpathway of glycerol degradation to 1,3-propanediol); PWY-6071 (superpathway of phenylethylamine degradation); PWY0-1277 (3-phenylpropanoate and 3-(3-hydroxyphenyl) propanoate degradation); and PWY-6690 (cinnamate and 3-hydroxycinnamate degradation to 2-oxopent-4-enoate), is indicative of non-CDI causative diarrhea associated with IBS.
Aspect 115) The method of aspect 112-114, wherein a change in relative abundance of at least four metabolic features is indicative of non-CDI causative diarrhea associated with IBS.
Aspect 116) The method of aspect 70, wherein the measuring of one or more metabolic features from a biological sample from the individual comprises determining changes in relative abundance, compared to a reference dysbiosis gut microbiome from individuals with CDI, of at least one of the pathways identified in Table 3 as being upregulated greater than 5 fold; wherein the change in metabolic feature relative abundance is indicative of non-CDI causative diarrhea associated with IBD.
Aspect 117) The method of aspect 116, wherein an increase in relative abundance of at least one of: PWY-6151 (S-adenosyl-L-methionine cycle I); P221-PWY (octane oxidation); PWY-5505 (L-glutamate and L-glutamine biosynthesis); PWY-5121 (superpathway of geranylgeranyl diphosphate biosynthesis II (via MEP)); GLYCOGENSYNTH-PWY (glycogen biosynthesis I (from ADP-D-Glucose)); NONMEVIPP-PWY (methylerythritol phosphate pathway I); PWY-6269 (adenosylcobalamin salvage from cobinamide II); PYRIDNUCSYN-PWY (NAD biosynthesis I (from aspartate)); COA-PWY (coenzyme A biosynthesis I); PWY-5686 (UMP biosynthesis); PWY-7560 (methylerythritol phosphate pathway II); PWY-6386 (UDP-N-acetylmuramoyl-pentapeptide biosynthesis II (lysine-containing)); PWY-6277 (superpathway of 5-aminoimidazole ribonucleotide biosynthesis); PWY-5509 (adenosylcobalamin biosynthesis from cobyrinate a,c-diamide I); PWY-7219 (adenosine ribonucleotides de novo biosynthesis); COBALSYN-PWY (adenosylcobalamin salvage from cobinamide I); PWY-6163 (chorismate biosynthesis from 3-dehydroquinate); PWY-6123 (inosine-5′-phosphate biosynthesis I); GLYCOCAT-PWY (glycogen degradation I (bacterial)); ARO-PWY (chorismate biosynthesis I); PWY-6317 (galactose degradation I (Leloir pathway)); PWY-5097 (L-lysine biosynthesis VI); PWY-6385 (peptidoglycan biosynthesis III (mycobacteria)); COMPLETE-ARO-PWY (superpathway of aromatic amino acid biosynthesis); PWY-6892 (thiazole biosynthesis I (E. coli)); PEPTIDOGLYCANSYN-PWY (peptidoglycan biosynthesis I (meso-diaminopimelate containing)); PWY-6122 (5-aminoimidazole ribonucleotide biosynthesis II); PYRIDNUCSAL-PWY (NAD salvage pathway I); PWY-5667 (CDP-diacylglycerol biosynthesis I); PWY-6387 (UDP-N-acetylmuramoyl-pentapeptide biosynthesis I (meso-diaminopimelate containing)); TRNA-CHARGING-PWY (IRNA charging); PWY-5100 (pyruvate fermentation to acetate and lactate II); PWY-7663 (gondoate biosynthesis (anaerobic)); PWY-5104 (L-isoleucine biosynthesis IV); PWY-5973 (cis-vaccenate biosynthesis); PWY0-1319 (CDP-diacylglycerol biosynthesis II); PWY-7221 (guanosine ribonucleotides de novo biosynthesis); PWY4FS-8 (phosphatidylglycerol biosynthesis II (non-plastidic)); PWY-6737 (starch degradation V); PWY-3001 (superpathway of L-isoleucine biosynthesis I); HISTSYN-PWY (L-histidine biosynthesis); PWY-7208 (superpathway of pyrimidine nucleobases salvage); PHOSLIPSYN-PWY (superpathway of phospholipid biosynthesis I (bacteria)); PWY-6121 (5-aminoimidazole ribonucleotide biosynthesis I); PWY-2942 (L-lysine biosynthesis III); SER-GLYSYN-PWY (superpathway of L-serine and glycine biosynthesis I); AEROBACTINSYN-PWY (acrobactin biosynthesis); PWY-6126 (superpathway of adenosine nucleotides de novo biosynthesis II); DAPLYSINESYN-PWY (L-lysine biosynthesis I); NONOXIPENT-PWY (pentose phosphate pathway (non-oxidative branch)); BRANCHED-CHAIN-AA-SYN-PWY (superpathway of branched amino acid biosynthesis); PWY-7229 (superpathway of adenosine nucleotides de novo biosynthesis I); ARGSYNBSUB-PWY (L-arginine biosynthesis II (acetyl cycle)); VALSYN-PWY (L-valine biosynthesis); ILEUSYN-PWY (L-isoleucine biosynthesis I (from threonine)); 1CMET2-PWY (N10-formyl-tetrahydrofolate biosynthesis); GALLATE-DEGRADATION-II-PWY (gallate degradation I); 3-HYDROXYPHENYLACETATE-DEGRADATION-PWY (4-hydroxyphenylacetate degradation); METHYLGALLATE-DEGRADATION-PWY (methylgallate degradation); PWY-7400 (L-arginine biosynthesis IV (archaebacteria)); THRESYN-PWY (superpathway of L-threonine biosynthesis); DENOVOPURINE2-PWY (superpathway of purine nucleotides de novo biosynthesis II); ARGSYN-PWY (L-arginine biosynthesis I (via L-ornithine)); PWY-7210 (pyrimidine deoxyribonucleotides biosynthesis from CTP); PWY-7198 (pyrimidine deoxyribonucleotides de novo biosynthesis IV); POLYAMINSYN3-PWY (superpathway of polyamine biosynthesis II); PWY4FS-7 (phosphatidylglycerol biosynthesis I (plastidic)); PWY0-166 (superpathway of pyrimidine deoxyribonucleotides de novo biosynthesis (E. coli)); GALLATE-DEGRADATION-I-PWY (gallate degradation II); CALVIN-PWY (Calvin-Benson-Bassham cycle); PANTOSYN-PWY (pantothenate and coenzyme A biosynthesis I); PWY-5484 (glycolysis II (from fructose 6-phosphate)); PWY-7184 (pyrimidine deoxyribonucleotides de novo biosynthesis I); HEMESYN2-PWY (heme biosynthesis II (anaerobic)); and POLYISOPRENSYN-PWY (polyisoprenoid biosynthesis (E. coli)), is indicative of non-CDI causative diarrhea associated with IBD.
Aspect 118) The method of any one of aspects 116-117, wherein a change in relative abundance of at least four metabolic features is indicative of non-CDI causative diarrhea associated with IBD.
Aspect 119) The method of aspect 70, wherein the measuring of one or more metabolic features from a biological sample from the individual comprises determining relative changes in abundance, compared to a reference gut microbiome, in at least one of: AST-PWY (L-arginine degradation II (AST pathway)); ECASYN-PWY (enterobacterial common antigen biosynthesis); THREOCAT-PWY (superpathway of L-threonine metabolism); PPGPPMET-PWY (ppGpp biosynthesis); PWY0-1338 (polymyxin resistance); PWY-6263 (superpathway of menaquinol-8 biosynthesis II); PWY-7371 (1,4-dihydroxy-6-naphthoate biosynthesis II); PWY-7374 (1,4-dihydroxy-6-naphthoate biosynthesis I); P221-PWY (octane oxidation); PWY-6749 (CMP-legionaminate biosynthesis I); PWY-7456 (mannan degradation); NONMEVIPP-PWY (methylerythritol phosphate pathway I); PWY-5097 (L-lysine biosynthesis VI); PWY-5505 (L-glutamate and L-glutamine biosynthesis); PWY-6122 (5-aminoimidazole ribonucleotide biosynthesis II); PWY-7663 (gondoate biosynthesis (anaerobic)); THRESYN-PWY (superpathway of L-threonine biosynthesis); HEMESYN2-PWY (heme biosynthesis II (anaerobic)); PWY-5304 (superpathway of sulfur oxidation (archaca); PWY-6478 (GDP-D-glycero-alpha-D-manno-heptose biosynthesis); PWY-7198 (pyrimidine deoxyribonucleotides de novo biosynthesis IV); and PWY-7210 (pyrimidine deoxyribonucleotides biosynthesis from CTP); wherein increased abundance of at least one of AST-PWY (L-arginine degradation II (AST pathway)); ECASYN-PWY (enterobacterial common antigen biosynthesis); THREOCAT-PWY (superpathway of L-threonine metabolism); PPGPPMET-PWY (ppGpp biosynthesis); and PWY0-1338 (polymyxin resistance) is associated with CDI causative diarrhea and the individual is treated accordingly; wherein increased abundance of at least one of PWY-6263 (superpathway of menaquinol-8 biosynthesis II); PWY-7371 (1,4-dihydroxy-6-naphthoate biosynthesis II); PWY-7374 (1,4-dihydroxy-6-naphthoate biosynthesis I); P221-PWY (octane oxidation); PWY-6749 (CMP-legionaminate biosynthesis I); and PWY-7456 (mannan degradation) is associated with IBD UC causative diarrhea and the individual is treated accordingly; and wherein increased abundance of at least one of NONMEVIPP-PWY (methylerythritol phosphate pathway I); PWY-5097 (L-lysine biosynthesis VI); PWY-5505 (L-glutamate and L-glutamine biosynthesis); PWY-6122 (5-aminoimidazole ribonucleotide biosynthesis II); PWY-7663 (gondoate biosynthesis (anaerobic)); THRESYN-PWY (superpathway of L-threonine biosynthesis); HEMESYN2-PWY (heme biosynthesis II (anaerobic)); PWY-5304 (superpathway of sulfur oxidation (archaca); PWY-6478 (GDP-D-glycero-alpha-D-manno-heptose biosynthesis); PWY-7198 (pyrimidine deoxyribonucleotides de novo biosynthesis IV); and PWY-7210 (pyrimidine deoxyribonucleotides biosynthesis from CTP) is associated with IBD causative diarrhea and the individual is treated accordingly.
Aspect 120) A method of diagnosing an individual having diarrhea comprising: measuring for presence or absence or a certain level of one or more taxonomical feature(s) from a biological sample from the individual; and classifying the individual as having a CDI associated pathogenic infection, irritable bowel syndrome (IBS), or inflammatory bowel disease (IBD).
Aspect 121) The method of any one of aspects 120, wherein the diagnosis is characterized by measuring the presence, absence, and/or relative quantity of at least 10 taxonomical features described in any one of Tables 9-17.
Aspect 122) The method of aspect 120, wherein the diagnosis is characterized by measuring the presence, absence, and/or relative quantity of at least 20 taxonomical features described in any one of Tables 9-17.
Aspect 123) The method of aspect 120, wherein the diagnosis is characterized by measuring the presence, absence, and/or relative quantity of at least 40 taxonomical features described in any one of Tables 9-17.
Aspect 124) The method of aspect 120, wherein the diagnosis is characterized by measuring the presence, absence, and/or relative quantity of at least 60 taxonomical features described in any one of Tables 9-17.
Aspect 125) The method of aspect 120, wherein the diagnosis is characterized by measuring the presence, absence, and/or relative quantity of at least 80 taxonomical features described in any one of Tables 9-17.
Aspect 126) The method of aspect 120, wherein diagnosis is characterized by measuring the presence, absence, and/or relative quantity of at least 100 taxonomical features described in any one Tables 9-17.
Aspect 127) The method of any one of aspects 121-126, wherein the diagnosis comprises characterization using more than one of Tables 9-17.
Aspect 128) The method of aspect 127, wherein the more than one characterization using Tables 9-17 is sequential.
Aspect 129) The method of aspect 127 or 128, wherein the more than one characterization using Tables 9-17 comprises first characterizing using Tables 10 and/or 12, followed by characterization using one or more of the remaining Tables.
Aspect 130) The method of any one of aspects 120-129, wherein the measuring of one or more taxonomical features comprises at least one of analyzing one or more nucleic acids in the sample, analyzing one or more metabolites in the sample, and analyzing one or more proteins in the sample.
Aspect 131) The method of aspect 130, wherein the nucleic acid is analyzed by sequencing, polymerase chain reaction, isothermal amplification, bioinformatics, or any combination thereof.
Aspect 132) The method of aspect 131, wherein the nucleic acid analyzed is 16S ribosomal RNA.
Aspect 133) The method of aspect 130, wherein the metabolites are analyzed by mass spectrometry, ELISA, chromatography, or any combination thereof.
Aspect 134) The method of aspect 130, wherein the proteins are analyzed by mass spectrometry, ELISA, chromatography, Western blotting, immunoprecipitation, immunoelectrophoresis, or any combination thereof.
Aspect 135) The method of any one of aspects 120-134, wherein the diagnosis is indicative of non-CDI causative diarrhea, the subject microbiome is further characterized to determine whether the non-CDI causative diarrhea is associated with irritable bowel syndrome (IBS) or inflammatory bowel disease (IBD),
Aspect 136) The method of aspect 135, wherein the non-CDI causative diarrhea is associated with IBD, the IBD is further characterized to determine whether the IBD is Ulcerative Colitis (UC) or Crohn's Disease (CD).
Aspect 137) The method of aspect 120, wherein the measuring of one or more taxonomical features from a biological sample from the individual comprises determining changes in relative abundance, compared to a reference healthy gut microbiome, of at least one of: Sphingobacteriaceae Pedobacter; Saccharibacteria Incertae Sedis; Peptostreptococcaceae Intestinibacter; Bacillales Incertae Sedis Gemella; Veillonella infantium; Chloroplast Streptophyta; Caulobacter segnis; Methylophilaceae Methylophilus; Lactobacillus Streptococcaceae; Burkholderiales Comamonadaceae; Burkholderia ambifaria; Caulobacteraceae Caulobacter; Betaproteobacteria; Phyllobacteriaceae Phyllobacterium; Burkholderiales Burkholderiaceae; Sphingomonadaceae; Sphingomonas; Prevotellamassilia timonensis; Collinsella aerofaciens; Adlercreutzia equolifaciens; Blautia hominis; and Dorea formicigenerans; wherein the change in taxonomical feature relative abundance is indicative of non-CDI causative diarrhea associated with IBS.
Aspect 138) The method of aspect 137, wherein an increase in relative abundance of at least one of: Sphingobacteriaceae Pedobacter; Saccharibacteria Incertae Sedis; Peptostreptococcaceae Intestinibacter; Bacillales Incertae Sedis Gemella; Veillonella infantium; Chloroplast Streptophyta; Caulobacter segnis; Methylophilaceae Methylophilus; Lactobacillus Streptococcaceae; Burkholderiales Comamonadaceae; Burkholderia ambifaria; Caulobacteraceae Caulobacter; Betaproteobacteria; Phyllobacteriaceae Phyllobacterium; Burkholderiales Burkholderiaceae; Sphingomonadaceae; Sphingomonas; and Prevotellamassilia timonensis, is indicative of non-CDI causative diarrhea associated with IBS.
Aspect 139) The method of aspect 137, wherein a decrease in relative abundance of at least one of: Collinsella acrofaciens; Adlercreutzia equolifaciens; Blautia hominis; and Dorea formicigenerans, is indicative of non-CDI causative diarrhea associated with IBS.
Aspect 140) The method of aspect 137-139, wherein a change in relative abundance of at least four taxonomical features is indicative of non-CDI causative diarrhea associated with IBS.
Aspect 141) The method of aspect 120, wherein the measuring of one or more taxonomical features from a biological sample from the individual comprises determining changes in relative abundance, compared to a reference healthy gut microbiome, of at least one of: Acidaminococcaceae Acidaminococcus; Actinomycetales Actinomycetaceae; Bacillales Gemella; Bacilli Lactobacillus; Betaproteobacteria; Betaproteobacteria Burkholderiales; Bifidobacterium boum; Blautia hominis; Clostridiales Incertae Sedis XI Parvimonas; Enterobacteriaceae Cedecea; Erysipelotrichaceae Faecalicoccus; Faecalimonas umbilicata; Fusobacteriaceae Fusobacterium; Fusobacterium nucleatum; Gammaproteobacteria Enterobacterales; Lachnoclostridium [Clostridium] bolteae; Lachnospiraceae Lachnoclostridium; Megasphaera micronuciformis; Peptoniphilaceae Peptoniphilus; Peptostreptococcaceae Peptostreptococcus; Peptostreptococcaceae Romboutsia; Peptostreptococcaceae Terrisporobacter; Proteobacteria Gammaproteobacteria; Rosenbergiella collisarenosi; Rosenbergiella nectarea; Ruminococcaceae Intestinimonas; Ruminococcaceae Subdoligranulum; Solobacterium moorei; Veillonellaceae Veillonella; Alistipes shahii; Bacteroides cellulosilyticus; Bacteroides koreensis; Bacteroides thetaiotaomicron; Bacteroides xylanisolvens; Colidextribacter massiliensis; Coprococcus catus; Coprococcus eutactus; Enterobacterales Enterobacteriaceae; Faecalibacterium prausnitzii; Methanobrevibacter smithii; Phascolarctobacterium faecium; Romboutsia timonensis; and Turicibacter sanguinis; wherein the change in taxonomical feature relative abundance is indicative of non-CDI causative diarrhea associated with IBD.
Aspect 142) The method of aspect 141, wherein an increase in relative abundance of at least one of: Acidaminococcaceae Acidaminococcus; Actinomycetales Actinomycetaceae; Bacillales Gemella; Bacilli Lactobacilluses; Betaproteobacteria; Betaproteobacteria Burkholderiales; Bifidobacterium boum; Blautia hominis; Clostridiales Incertae Sedis XI Parvimonas; Enterobacteriaceae Cedecea; Erysipelotrichaceae Faecalicoccus; Faecalimonas umbilicata; Fusobacteriaceae Fusobacterium; Fusobacterium nucleatum; Gammaproteobacteria Enterobacterales; Lachnoclostridium [Clostridium] bolteae; Lachnospiraceae Lachnoclostridium; Megasphaera micronuciformis; Peptoniphilaceae Peptoniphilus; Peptostreptococcaceae Peptostreptococcus; Peptostreptococcaceae Romboutsia; Peptostreptococcaceae Terrisporobacter; Proteobacteria Gammaproteobacteria; Rosenbergiella collisarenosi; Rosenbergiella nectarea; Ruminococcaceae Intestinimonas; Ruminococcaceae Subdoligranulum; Solobacterium moorei; and Veillonellaceae Veillonella, is indicative of non-CDI causative diarrhea associated with IBD.
Aspect 143) The method of aspect 141, wherein a decrease in relative abundance of at least one of: Alistipes shahii; Bacteroides cellulosilyticus; Bacteroides koreensis; Bacteroides thetaiotaomicron; Bacteroides xylanisolvens; Colidextribacter massiliensis; Coprococcus catus; Coprococcus eutactus; Enterobacterales Enterobacteriaceae; Faecalibacterium prausnitzii; Methanobrevibacter smithii; Phascolarctobacterium faecium; Romboutsia timonensis; and Turicibacter sanguinis, is indicative of non-CDI causative diarrhea associated with IBD.
Aspect 144) The method of aspect 141-143, wherein a change in relative abundance of at least four taxonomical features is indicative of non-CDI causative diarrhea associated with IBD.
Aspect 145) The method of aspect 120, wherein the individual is a pediatric individual, and wherein the measuring of one or more taxonomical features from a biological sample from the individual comprises determining changes in relative abundance, compared to a reference healthy pediatric gut microbiome, of at least one of: Bacilli Lactobacilluses; Peptostreptococcaceae Clostridioides; Enterococcaceae Enterococcus; Eggerthellaceae Eggerthella; Erysipelotrichaceae Erysipelatoclostridium; Lachnospiraceae Lachnoclostridium; Firmicutes Bacilli; Enterobacteriaceae Escherichia; Enterobacteriales Enterobacteriaceae; Streptococcaceae Streptococcus; Actinomycetaceae Schaalia; Clostridiaceae Hungatella; Clostridiaceae Clostridium; Corynebacteriaceae Corynebacterium; Veillonellaceae Veillonella; Actinomycetaceae Actinomyces; Fusobacteriaceae Fusobacterium; Erysipelotrichaceae Longicatena; Lachnospiraceae Clostridium XlVa; Lachnospiraceae Eisenbergiella; Peptostreptococcaceae Intestinibacter; Enterobacteriaceae Citrobacter; Peptoniphilaceae Finegoldia; Carnobacteriaceae Granulicatella; Coriobacteriaceae Eggerthella; Peptostreptococcaceae Peptostreptococcus; Drancourtella massiliensis; Peptoniphilaceae Anaerococcus; Bacillales Gemella; Lachnospiraceae Sellimonas; Peptoniphilaceae Peptoniphilus; Pasteurellaceae Rodentibacter; Clostridium sensu stricto; Lachnospiraceae Faecalimonas; Proteobacteria Gammaproteobacteria; Clostridiaceae Lactonifactor; Micrococcaceae Rothia; Leuconostocaceae Weissella; Peptostreptococcaceae Terrisporobacter; Peptostreptococcaceae Romboutsia; Peptostreptococcaceae Clostridium XI; Coriobacteriaceae Atopobium; Atopobiaceae Atopobium; Anaeromassilibacillus; Ruminococcaceae Porphyromonadaceae Parabacteroides; Rikenellaceae Alistipes; Lachnospiraceae Eubacterium rectale group; Lachnospiraceae Coprococcus; Lachnospiraceae Roseburia; Porphyromonadacede Odoribacter; Veillonellaceae Dialister; Ruminococcaceae Clostridium IV; Bacteroidia Bacteroidales; Coriobacteriaceae Collinsella; Coriobacteriales Coriobacteriaceae; Ruminococcaceae Clostridium leptum group; Enterobacteriaceae Shimwellia; Acidaminococcaceae Phascolarctobacterium; Staphylococcaceae Staphylococcus; Lachnospiraceae Anaerobutyricum; Erysipelotrichaceae Holdemania; Clostridiales Monoglobus; Porphyromonadaceae Barnesiella; Pasteurellales Pasteurellaceae; Clostridiales Clostridiaceae 1; and Bacteroidales Rikenellaceae; wherein the change in taxonomical feature relative abundance is indicative of CDI associated diarrhea.
Aspect 146) The method of aspect 145, wherein an increase in relative abundance of at least one of: Bacilli Lactobacillus; Peptostreptococcaceae Clostridioides; Enterococcaceae Enterococcus; Eggerthellaceae Eggerthella; Erysipelotrichaceae Erysipelatoclostridium; Lachnospiraceae Lachnoclostridium; Firmicutes Bacilli; Enterobacteriaceae Escherichia; Enterobacteriales Enterobacteriaceae; Streptococcus; Actinomycetaceae Streptococcaceae Schaalia; Clostridiaceae Hungatella; Clostridiaceae Clostridium; Corynebacteriaceae Corynebacterium; Veillonellaceae Veillonella; Actinomycetaceae Actinomyces; Fusobacteriaceae Fusobacterium; Erysipelotrichaceae Longicatena; Lachnospiraceae Clostridium XIVa; Lachnospiraceae Eisenbergiella; Peptostreptococcaceae Intestinibacter; Enterobacteriaceae Citrobacter; Peptoniphilaceae Finegoldia; Carnobacteriaceae Granulicatella; Coriobacteriaceae Eggerthella; Peptostreptococcaceae Peptostreptococcus; Drancourtella massiliensis; Peptoniphilaceae Anaerococcus; Bacillales Gemella; Lachnospiraceae Sellimonas; Peptoniphilaceae Peptoniphilus; Pasteurellaceae Rodentibacter; Clostridium sensu stricto; Lachnospiraceae Faecalimonas; Proteobacteria Gammaproteobacteria; Clostridiaceae Lactonifactor; Micrococcaceae Rothia; Leuconostocaceae Weissella; Peptostreptococcaceae Terrisporobacter; Peptostreptococcaceae Romboutsia; Peptostreptococcaceae Clostridium XI; Coriobacteriaceae Atopobium; Atopobiaceae Atopobium; and Ruminococcaceae Anaeromassilibacillus, is indicative of CDI associated diarrhea.
Aspect 147) The method of aspect 145, wherein a decrease in relative abundance of at least one of: Porphyromonadaceae Parabacteroides; Rikenellaceae Alistipes; Lachnospiraceae Eubacterium rectale group; Lachnospiraceae Coprococcus; Lachnospiraceae Roseburia; Porphyromonadacede Odoribacter; Veillonellaceae Dialister; Ruminococcaceae Clostridium IV; Bacteroidia Bacteroidales; Coriobacteriaceae Collinsella; Coriobacteriales Coriobacteriaceae; Ruminococcaceae Clostridium leptum group; Enterobacteriaceae Shimwellia; Acidaminococcaceae Phascolarctobacterium; Staphylococcaceae Staphylococcus; Lachnospiraceae Anaerobutyricum; Erysipelotrichaceae Holdemania; Clostridiales Monoglobus; Porphyromonadacede Barnesiella; Pasteurellales Pasteurellaceae; Clostridiales Clostridiaceae 1; and Bacteroidales Rikenellaceae, is indicative of CDI associated diarrhea.
Aspect 148) The method of aspect 145-147, wherein a change in relative abundance of at least four taxonomical features is indicative of CDI associated diarrhea.
Aspect 149) The method of aspect 135, wherein the characterization of the subject microbiome to determine whether the non-CDI causative diarrhea is associated with IBS or IBD comprises measuring one or more taxonomical features from a biological sample from the subject and determining changes in relative abundance, compared to a reference dysbiosis IBD gut microbiome, of at least one of: Blautia stercoris; Saccharibacteria Incertae Sedis; Bacteroides plebeius; Bacteroides nordii; Eubacterium siraeum; Bacteroides cellulosilyticus; Burkholderiales Burkholderiaceae; Caulobacteraceae Caulobacter; Pelomonas aquatic; Burkholderiaceae Burkholderia; Caulobacter segnis; Burkholderia ambifaria; Eubacterium ventriosum; Firmicutes Clostridia; Oxalobacter formigenes; Burkholderiales Comamonadaceae; Veillonella infantium; Bacteroides thetaiotaomicron; Sphingobacteriaceae Pedobacter; Burkholderia thailandensis; Erysipelotrichaceae Turicibacter; Collinsella aerofaciens; Veillonellaceae Veillonella; Lachnospiraceae Lachnoclostridium; Blautia hominis; Gammaproteobacteria Enterobacterales; Bifidobacteriales Bifidobacteriaceae; Ruminococcaceae Intestinimonas; Faecalimonas umbilicata; Actinomycetales Actinomycetaceae; Actinobacteria Actinobacteria; Dorea formicigenerans; and Bacteria Proteobacteria; wherein the change in taxonomical feature relative abundance is indicative of non-CDI causative diarrhea associated with IBS.
Aspect 150) The method of aspect 149, wherein an increase in relative abundance of at least one of: Saccharibacteria Incertae Sedis; Bacteroides plebeius; Bacteroides nordii; Eubacterium siraeum; Bacteroides cellulosilyticus; Burkholderiales Burkholderiaceae; Caulobacteraceae Caulobacter; Pelomonas aquatic; Burkholderiaceae Burkholderia; Caulobacter segnis; Burkholderia ambifaria; Eubacterium ventriosum; Firmicutes Clostridia; Oxalobacter formigenes; Burkholderiales Comamonadaceae; Veillonella infantium; Bacteroides thetaiotaomicron; Sphingobacteriaceae Pedobacter; Burkholderia thailandensis; and Erysipelotrichaceae Turicibacter, is indicative of non-CDI causative diarrhea associated with IBS.
Aspect 151) The method of aspect 149, wherein a decrease in relative abundance of at least one of: Collinsella aerofaciens; Veillonellaceae Veillonella; Lachnospiraceae Lachnoclostridium; Blautia hominis; Gammaproteobacteria Enterobacterales; Bifidobacteriales Bifidobacteriaceae; Ruminococcaceae Intestinimonas; Faecalimonas umbilicata; Actinomycetales Actinomycetaceae; Actinobacteria Actinobacteria; Dorea formicigenerans; and Bacteria Proteobacteria, is indicative of non-CDI causative diarrhea associated with IBS.
Aspect 152) The method of aspect 149-151, wherein a change in relative abundance of at least four taxonomical features is indicative of non-CDI causative diarrhea associated with IBS.
Aspect 153) The method of aspect 136, wherein the characterization of the subject microbiome to determine whether the non-CDI causative diarrhea is associated with IBD UC or IBD CD comprises measuring one or more taxonomical features from a biological sample from the subject and determining changes in relative abundance, compared to a reference healthy gut microbiome, of at least one of: Lachnospiraceae Lachnoclostridium; Ruminococcaceae Intestinimonas; Acidaminococcaceae Acidaminococcus; Bacilli Lactobacillus; Actinomycetales Actinomycetaceae; Ruminococcaceae Subdoligranulum; Peptostreptococcaceae Romboutsia; Bifidobacterium boum; Bacillales Gemella; Peptostreptococcaceae Peptostreptococcus; Peptoniphilaceae Peptoniphilus; Clostridiales Incertae Sedis XI Parvimonas; Peptostreptococcaceae Terrisporobacter; Erysipelotrichaceae Faecalicoccus; Solobacterium moorei; Bacteroides xylanisolvens; Enterobacterales Enterobacteriaceae; Bacteroides koreensis; Bacteroides cellulosilyticus; Phascolarctobacterium faecium; and Bacteroides thetaiotaomicron; wherein the change in taxonomical feature relative abundance is indicative of non-CDI causative diarrhea associated with IBD UC.
Aspect 154) The method of aspect 153, wherein an increase in relative abundance of at least one of: Lachnospiraceae Lachnoclostridium; Ruminococcaceae Intestinimonas; Acidaminococcaceae Acidaminococcus; Bacilli Lactobacilluses; Actinomycetales Actinomycetaceae; Ruminococcaceae Subdoligranulum; Peptostreptococcaceae Romboutsia; Bifidobacterium boum; Bacillales Gemella; Peptostreptococcaceae Peptostreptococcus; Peptoniphilaceae Peptoniphilus; Clostridiales Incertae Sedis XI Parvimonas; Peptostreptococcaceae Terrisporobacter; Erysipelotrichaceae Faecalicoccus; and Solobacterium moorei, is indicative of non-CDI causative diarrhea associated with IBD UC.
Aspect 155) The method of aspect 153, wherein a decrease in relative abundance of at least one of: Bacteroides xylanisolvens; Enterobacterales Enterobacteriaceae; Bacteroides koreensis; Bacteroides cellulosilyticus; Phascolarctobacterium faecium; and Bacteroides thetaiotaomicron, is indicative of non-CDI causative diarrhea associated with IBD UC.
Aspect 156) The method of aspect 153-155, wherein a change in relative abundance of at least four taxonomical features is indicative of non-CDI causative diarrhea associated with IBD UC.
Aspect 157) The method of aspect 136, wherein the characterization of the subject microbiome to determine whether the non-CDI causative diarrhea is associated with IBD UC or IBD CD comprises measuring one or more taxonomical features from a biological sample from the subject and determining changes in relative abundance, compared to a reference healthy gut microbiome, of at least one of: Faecalimonas umbilicata; Lachnospiraceae Lachnoclostridium; Lachnoclostridium [Clostridium] bolteae; Proteobacteria Gammaproteobacteria; Bacilli Lactobacillus; Actinomycetales Actinomycetaceae; Gammaproteobacteria Enterobacterales; Rosenbergiella collisarenosi; Rosenbergiella nectarea; Veillonellaceae Veillonella; Acidaminococcaceae Acidaminococcus; Blautia hominis; Bifidobacterium boum; Enterobacteriaceae Cedecea; Ruminococcaceae Intestinimonas; Peptostreptococcaceae Romboutsia; Fusobacteriaceae Fusobacterium; Fusobacterium nucleatum; Megasphaera micronuciformis; Romboutsia timonensis; Faecalibacterium prausnitzii; Coprococcus catus; Alistipes shahii; Bacteroides cellulosilyticus; Bacteroides thetaiotaomicron; Coprococcus eutactus; Turicibacter sanguinis; Colidextribacter massiliensis; and Methanobrevibacter smithii; wherein the change in taxonomical feature relative abundance is indicative of non-CDI causative diarrhea associated with IBD CD.
Aspect 158) The method of aspect 157, wherein an increase in relative abundance of at least one of: Faecalimonas umbilicata; Lachnospiraceae Lachnoclostridium; Lachnoclostridium [Clostridium] bolteae; Proteobacteria Gammaproteobacteria; Bacilli Lactobacillus; Actinomycetales Actinomycetaceae; Gammaproteobacteria Enterobacterales; Rosenbergiella collisarenosi; Rosenbergiella nectarea; Veillonellaceae Veillonella; Acidaminococcaceae Acidaminococcus; Blautia hominis; Bifidobacterium boum; Enterobacteriaceae Cedecea; Ruminococcaceae Intestinimonas; Peptostreptococcaceae Romboutsia; Fusobacteriaceae Fusobacterium; Fusobacterium nucleatum; and Megasphaera micronuciformis, is indicative of non-CDI causative diarrhea associated with IBD CD.
Aspect 159) The method of aspect 157, wherein a decrease in relative abundance of at least one of: Romboutsia timonensis; Faecalibacterium prausnitzii; Coprococcus catus; Alistipes shahii; Bacteroides cellulosilyticus; Bacteroides thetaiotaomicron; Coprococcus eutactus; Turicibacter sanguinis; Colidextribacter massiliensis; and Methanobrevibacter smithii, is indicative of non-CDI causative diarrhea associated with IBD CD.
Aspect 160) The method of aspect 157-159, wherein a change in relative abundance of at least four taxonomical features is indicative of non-CDI causative diarrhea associated with IBD CD.
Aspect 161) The method of aspect 136, wherein the characterization of the subject microbiome to determine whether the non-CDI causative diarrhea is associated with IBD UC or IBD CD comprises measuring one or more taxonomical features from a biological sample from the subject and determining changes in relative abundance, compared to a reference dysbiosis IBD UC gut microbiome, of at least one of: Faecalimonas umbilicata; Blautia [Ruminococcus] gnavus; Lachnoclostridium [Clostridium] boltede; Erysipelatoclostridium ramosum; Veillonellaceae Veillonella; Enterobacterales Enterobacteriaceae; Blautia caecimuris; Fusobacteriaceae Fusobacterium; Fusobacterium nucleatum; Faecalibacterium prausnitzii; Ruminococcaceae Oscillibacter; Ruminococcaceae Clostridium IV; Romboutsia timonensis; Ruminococcaceae Pseudoflavonifractor; Erysipelotrichales Erysipelotrichaceae; and Methanobacteriaceae Methanobrevibacter; wherein the change in taxonomical feature relative abundance is indicative of non-CDI causative diarrhea associated with IBD CD.
Aspect 162) The method of aspect 161, wherein an increase in relative abundance of at least one of: Faecalimonas umbilicata; Blautia [Ruminococcus] gnavus; Lachnoclostridium [Clostridium] boltede; Erysipelatoclostridium ramosum; Veillonellaceae Veillonella; Enterobacterales Enterobacteriaceae; Blautia caecimuris; Fusobacteriaceae Fusobacterium; and Fusobacterium nucleatum, is indicative of non-CDI causative diarrhea associated with IBD CD.
Aspect 163) The method of aspect 161, wherein a decrease in relative abundance of at least one of: Faecalibacterium prausnitzii; Ruminococcaceae Oscillibacter; Ruminococcaceae Clostridium IV; Romboutsia timonensis; Ruminococcaceae Pseudoflavonifractor; Erysipelotrichales Erysipelotrichaceae; and Methanobacteriaceae Methanobrevibacter, is indicative of non-CDI causative diarrhea associated with IBD CD.
Aspect 164) The method of aspect 161-163, wherein a change in relative abundance of at least four taxonomical features is indicative of non-CDI causative diarrhea associated with IBD CD.
Aspect 165) The method of aspect 120, wherein the measuring of one or more taxonomical features from a biological sample from the individual comprises determining changes in relative abundance, compared to a reference dysbiosis gut microbiome from individuals with CDI, of at least one of: Anaerostipes hadrus; Faecalibacterium prausnitzii; Lachnospiraceae Coprococcus; Lachnospiraceae [Eubacterium] rectale; Lachnospiraceae Roseburia; Lachnospiraceae Fusicatenibacter; Lachnospiraceae Dorea; Ruminococcaceae Ruminococcus; Roseburia inulinivorans; Dorea longicatena; Roseburia intestinalis; Lachnospiraceae Anaerostipes; Coprococcus comes; Lactobacillus rogosae; Romboutsia timonensis; Saccharibacteria Incertae Sedis; Eubacterium [Eubacterium] eligens; Bacteroides plebeius; Lachnospiraceae Blautia; Roseburia faecis; Blautia obeum; Coprococcus catus; Betaproteobacteria Burkholderiales; Prevotella copri; Burkholderiales Burkholderiaceae; Caulobacter segnis; Caulobacterales Caulobacteraceae; Burkholderiaceae Burkholderia; Burkholderia ambifaria; Prevotellaceae Prevotella; Burkholderiales Comamonadaceae; Burkholderia thailandensis; Alistipes obesi; Blautia stercoris; Ruminococcus bromii; Pelomonas aquatica; Peptostreptococcaceae Romboutsia; Bacteroides coprocola; Streptococcus thermophilus; Ruminococcaceae Oscillibacter; Alistipes ihumii; Ruminococcus callidus; Phyllobacteriaceae Phyllobacterium; Coprococcus eutactus; Peptostreptococcaceae Intestinibacter; Clostridioides difficile; Enterobacterales Enterobacteriaceae; Enterococcaceae Enterococcus; Peptostreptococcaceae Clostridium XI; Gammaproteobacteria Enterobacterales; Bacilli Lactobacillus; Eggerthella lenta; Erysipelatoclostridium [Clostridium] innocuum; Enterobacteriaceae Citrobacter; Lachnospiraceae Clostridium XIVa; Lachnoclostridium [Clostridium] bolteae; Erysipelatoclostridium ramosum; Hungatella effluvia; Veillonella parvula; Lactobacilluseae Lactobacillus; Blautia [Ruminococcus] gnavus; Enterococcus saccharolyticus; and Coriobacteriaceae Eggerthella; wherein the change in taxonomical feature relative abundance is indicative of non-CDI causative diarrhea associated with IBS.
Aspect 166) The method of aspect 165, wherein an increase in relative abundance of at least one of: Anaerostipes hadrus; Faecalibacterium prausnitzii; Lachnospiraceae Coprococcus; Lachnospiraceae [Eubacterium] rectale; Lachnospiraceae Roseburia; Lachnospiraceae Fusicatenibacter; Lachnospiraceae Dorea; Ruminococcaceae Ruminococcus; Roseburia inulinivorans; Dorea longicatena; Roseburia intestinalis; Lachnospiraceae Anaerostipes; Coprococcus comes; Lactobacillus rogosae; Romboutsia timonensis; Saccharibacteria Incertae Sedis; Eubacterium [Eubacterium] eligens; Bacteroides plebeius; Lachnospiraceae Blautia; Roseburia faecis; Blautia obeum; Coprococcus catus; Betaproteobacteria Burkholderiales; Prevotella copri; Burkholderiales Burkholderiaceae; Caulobacter segnis; Caulobacterales Caulobacteraceae; Burkholderiaceae Burkholderia; Burkholderia ambifaria; Prevotellaceae Prevotella; Burkholderiales Comamonadaceae; Burkholderia thailandensis; Alistipes obesi; Blautia stercoris; Ruminococcus bromii; Pelomonas aquatica; Peptostreptococcaceae Romboutsia; Bacteroides coprocola; Streptococcus thermophilus; Ruminococcaceae Oscillibacter; Alistipes ihumii; Ruminococcus callidus; Phyllobacteriaceae Phyllobacterium; Coprococcus eutactus; and Peptostreptococcaceae Intestinibacter, is indicative of non-CDI causative diarrhea associated with IBS.
Aspect 167) The method of aspect 165, wherein a decrease in relative abundance of at least one of: Clostridioides difficile; Enterobacterales Enterobacteriaceae; Enterobacteriales Enterobacteriaceae; Enterococcaceae Enterococcus; Peptostreptococcaceae Clostridium XI; Gammaproteobacteria Enterobacterales; Bacilli Lactobacilluses; Eggerthella lenta; Erysipelatoclostridium [Clostridium] innocuum; Enterobacteriaceae Citrobacter; Lachnospiraceae Clostridium XIVa; Lachnoclostridium [Clostridium] bolteae; Erysipelatoclostridium ramosum; Hungatella effluvia; Veillonella parvula; Lactobacilluseae Lactobacillus; Blautia [Ruminococcus] gnavus; Enterococcus saccharolyticus; and Coriobacteriaceae Eggerthella, is indicative of non-CDI causative diarrhea associated with IBS.
Aspect 168) The method of aspect 165-167, wherein a change in relative abundance of at least four taxonomical features is indicative of non-CDI causative diarrhea associated with IBS.
Aspect 169) The method of aspect 120, wherein the measuring of one or more taxonomical features from a biological sample from the individual comprises determining changes in relative abundance, compared to a reference dysbiosis gut microbiome from individuals with CDI, of at least one of: Anaerostipes hadrus; Lachnospiraceae Dorea; Dorea longicatena; Lachnospiraceae Coprococcus; Lachnospiraceae Roseburia; Blautia obeum; Coprococcus comes; Roseburia intestinalis; Lactobacillus rogosae; Eubacterium [Eubacterium] eligens; Bacteroidia Bacteroidales; Peptostreptococcaceae Romboutsia; Coriobacteriaceae Collinsella; Acidaminococcaceae Acidaminococcus; Prevotellaceae Prevotella; Prevotella copri; Coriobacteriales Coriobacteriaceae; Ruminococcaceae Ruminococcus; Alistipes obesi; Betaproteobacteria Burkholderiales; Ruminococcus bromii; Lachnospiraceae Lachnoclostridium; Bifidobacterium adolescentis; Actinomycetales Actinomycetaceae; Ruminococcaceae Intestinimonas; Bacteroides eggerthii; Clostridioides difficile; Peptostreptococcaceae Clostridium XI; Enterobacteriaceae Citrobacter; Enterobacterales Enterobacteriaceae; Veillonella parvula; Enterococcaceae Enterococcus; Lactobacillus Enterococcaceae; Erysipelatoclostridium [Clostridium] innocuum; Pectobacterium carotovorum; Enterobacteriales Enterobacteriaceae; Bacteroides xylanisolvens; Enterococcus saccharolyticus; Clostridium paraputrificum; Salmonella enterica; Erysipelatoclostridium ramosum; Hungatella effluvia; Bacteroides koreensis; Bacilli Lactobacillus; Blautia product; Klebsiella quasipneumoniae; Ruthenibacterium lactatiformans; Veillonella dispar; Coriobacteriaceae Eggerthella; and Lachnospiraceae Hungatella; wherein the change in taxonomical feature relative abundance is indicative of non-CDI causative diarrhea associated with IBD.
Aspect 170) The method of aspect 169, wherein an increase in relative abundance of at least one of: Anaerostipes hadrus; Lachnospiraceae Dorea; Dorea longicatena; Lachnospiraceae Coprococcus; Lachnospiraceae Roseburia; Blautia obeum; Coprococcus comes; Roseburia intestinalis; Lactobacillus rogosae; Eubacterium [Eubacterium] eligens; Bacteroidia Bacteroidales; Peptostreptococcaceae Romboutsia; Coriobacteriaceae Collinsella; Acidaminococcaceae Acidaminococcus; Prevotellaceae Prevotella; Prevotella copri; Coriobacteriales Coriobacteriaceae; Ruminococcaceae Ruminococcus; Alistipes obesi; Betaproteobacteria Burkholderiales; Ruminococcus bromii; Lachnospiraceae Lachnoclostridium; Bifidobacterium adolescentis; Actinomycetales Actinomycetaceae; Ruminococcaceae Intestinimonas; and Bacteroides eggerthii, is indicative of non-CDI causative diarrhea associated with IBD.
Aspect 171) The method of aspect 169, wherein a decrease in relative abundance of at least one of: Clostridioides difficile; Peptostreptococcaceae Clostridium XI; Enterobacteriaceae Citrobacter; Enterobacterales Enterobacteriaceae; Veillonella parvula; Enterococcaceae Enterococcus; Lactobacilluses Enterococcaceae; Erysipelatoclostridium [Clostridium] innocuum; Pectobacterium carotovorum; Enterobacteriales Enterobacteriaceae; Bacteroides xylanisolvens; Enterococcus saccharolyticus; Clostridium paraputrificum; Salmonella enterica; Erysipelatoclostridium ramosum; Hungatella effluvia; Bacteroides koreensis; Bacilli Lactobacillies; Blautia product; Klebsiella quasipneumoniae; Ruthenibacterium lactatiformans; Veillonella dispar; Coriobacteriaceae Eggerthella; and Lachnospiraceae Hungatella, is indicative of non-CDI causative diarrhea associated with IBD.
Aspect 172) The method of aspect 169-171, wherein a change in relative abundance of at least four taxonomical features is indicative of non-CDI causative diarrhea associated with IBD.
Aspect 173) A method of diagnosing an individual having diarrhea comprising: measuring for presence or absence or a certain level of one or more metabolic feature(s) from a biological sample from the individual; and classifying the individual as having a CDI associated pathogenic infection, irritable bowel syndrome (IBS), or inflammatory bowel disease (IBD).
Aspect 174) The method of aspect 173, wherein the diagnosing is characterized by measuring the presence, absence, and/or relative quantity of at least 10 metabolic features described in any one of Tables 1-8.
Aspect 175) The method aspect 174, wherein the diagnosing is characterized by measuring the presence, absence, and/or relative quantity of at least 20 metabolic features described in any one of Tables 1-8.
Aspect 176) The method of aspect 174, wherein the diagnosing is characterized by measuring the presence, absence, and/or relative quantity of at least 40 metabolic features described in any one of Tables 1-8.
Aspect 177) The method of aspect 174, wherein the diagnosing is characterized by measuring the presence, absence, and/or relative quantity of at least 60 metabolic features described in any one of Tables 1-8.
Aspect 178) The method of aspect 174, wherein the diagnosing is characterized by measuring the presence, absence, and/or relative quantity of at least 80 metabolic features described in any one of Tables 1-8.
Aspect 179) The method of aspect 174, wherein the diagnosing is characterized by measuring the presence, absence, and/or relative quantity of at least 100 metabolic features described in any one Tables 1-8.
Aspect 180) The method of any one of aspects 175-179, wherein the diagnosing comprises characterization using more than one of Tables 1-8.
Aspect 181) The method of aspect 180, wherein the more than one characterization using Tables 1-8 is sequential.
Aspect 182) The method of aspect 180 or 181, wherein the more than one characterization using Tables 1-8 comprises first characterizing using Tables 3 and/or 7, followed by characterization using one or more of the remaining Tables.
Aspect 183) The method of any one of aspects 173-182, wherein the measuring of one or more metabolic features comprises at least one of analyzing one or more nucleic acids in the sample, analyzing one or more metabolites in the sample, and analyzing one or more proteins in the sample.
Aspect 184) The method of aspect 183, wherein the nucleic acid is analyzed by sequencing, polymerase chain reaction, isothermal amplification, bioinformatics, or any combination thereof.
Aspect 185) The method of aspect 184, wherein the nucleic acid analyzed is 16S ribosomal RNA.
Aspect 186) The method of aspect 183, wherein the metabolites are analyzed by mass spectrometry, ELISA, chromatography, or any combination thereof.
Aspect 187) The method of aspect 183, wherein the proteins are analyzed by mass spectrometry, ELISA, chromatography, Western blotting, immunoprecipitation, immunoelectrophoresis, or any combination thereof.
Aspect 188) The method of any one of aspects 173-187, wherein the diagnosis is indicative of non-CDI causative diarrhea, the subject microbiome is further characterized to determine whether the non-CDI causative diarrhea is associated with irritable bowel syndrome (IBS) or inflammatory bowel disease (IBD),
Aspect 189) The method of aspect 188, wherein the non-CDI causative diarrhea is associated with IBD, the IBD is further characterized to determine whether the IBD is Ulcerative Colitis (UC) or Crohn's Disease (CD).
Aspect 190) The method of aspect 173, wherein the measuring of one or more metabolic features from a biological sample from the individual comprises determining changes in relative abundance, compared to a reference healthy gut microbiome, of at least one of: PWY-7431 (aromatic biogenic amine degradation (bacteria)); PWY-7159 (chlorophyllide a biosynthesis III (aerobic, light independent)); PWY-5531 (chlorophyllide a biosynthesis II (anaerobic)); CHLOROPHYLL-SYN (chlorophyllide a biosynthesis I (aerobic, light-dependent)); P562-PWY (myo-inositol degradation I); PWY-3781 (aerobic respiration I (cytochrome c)); TYRFUMCAT-PWY (L-tyrosine degradation I); HCAMHPDEG-PWY (3-phenylpropanoate and 3-(3-hydroxyphenyl) propanoate degradation to 2-oxopent-4-enoate); PWY-6071 (superpathway of phenylethylamine degradation); and PWY-6690 (cinnamate and 3-hydroxycinnamate degradation to 2-oxopent-4-enoate); wherein the change in metabolic feature relative abundance is indicative of non-CDI causative diarrhea associated with IBS.
Aspect 191) The method of aspect 190, wherein an increase in relative abundance of at least one of: PWY-7431 (aromatic biogenic amine degradation (bacteria)); PWY-7159 (chlorophyllide a biosynthesis III (aerobic, light independent)); PWY-5531 (chlorophyllide a biosynthesis II (anacrobic)); CHLOROPHYLL-SYN (chlorophyllide a biosynthesis I (aerobic, light-dependent)); P562-PWY (myo-inositol degradation I); PWY-3781 (aerobic respiration I (cytochrome c)); and TYRFUMCAT-PWY (L-tyrosine degradation I), is indicative of non-CDI causative diarrhea associated with IBS.
Aspect 192) The method of aspect 190, wherein a decrease in relative abundance of at least one of: HCAMHPDEG-PWY (3-phenylpropanoate and 3-(3-hydroxyphenyl) propanoate degradation to 2-oxopent-4-enoate); PWY-6071 (superpathway of phenylethylamine degradation); and PWY-6690 (cinnamate and 3-hydroxycinnamate degradation to 2-oxopent-4-enoate), is indicative of non-CDI causative diarrhea associated with IBS.
Aspect 193) The method of any one of aspects 190-192, wherein a change in relative abundance of at least three metabolic features is indicative of non-CDI causative diarrhea associated with IBS.
Aspect 194) The method of aspect 173, wherein the measuring of one or more metabolic features from a biological sample from the individual comprises determining changes in relative abundance, compared to a reference healthy gut microbiome, of at least one of: ASPASN-PWY (superpathway of L-aspartate and L-asparagine biosynthesis); DHGLUCONATE-PYR-CAT-PWY (glucose degradation (oxidative)); DTDPRHAMSYN-PWY (dTDP-L-rhamnose biosynthesis I); FASYN-ELONG-PWY (fatty acid elongation-saturated); GALACTARDEG-PWY (D-galactarate degradation I); GLUCARDEG-PWY (D-glucarate degradation I); GLUCARGALACTSUPER-PWY (superpathway of D-glucarate and D-galactarate degradation); GLUCONEO-PWY (gluconcogenesis I); GLUCUROCAT-PWY (superpathway of & beta; -D-glucuronide and D-glucuronate degradation); GLYCOLYSIS (glycolysis I (from glucose 6-phosphate)); GLYCOLYSIS-E-D (superpathway of glycolysis and Entner-Doudoroff); HEMESYN2-PWY (heme biosynthesis II (anaerobic)); HISDEG-PWY (L-histidine degradation I); METHGLYUT-PWY (superpathway of methylglyoxal degradation); P125-PWY (superpathway of (R,R)-butanediol biosynthesis); P162-PWY (L-glutamate degradation V (via hydroxyglutarate)); P163-PWY (L-lysine fermentation to acetate and butanoate); P42-PWY (incomplete reductive TCA cycle); PENTOSE-P-PWY (pentose phosphate pathway); POLYISOPRENSYN-PWY (polyisoprenoid biosynthesis (E. coli)); PWY0-1241 (ADP-L-glycero-& beta; -D-manno-heptose biosynthesis); PWY0-1533 (methylphosphonate degradation I); PWY0-41 (allantoin degradation IV (anaerobic)); PWY0-845 (superpathway of pyridoxal 5′-phosphate biosynthesis and salvage); PWY-5028 (L-histidine degradation II); PWY-5484 (glycolysis II (from fructose 6-phosphate)); PWY-5659 (GDP-mannose biosynthesis); PWY-5695 (urate biosynthesis/inosine 5′-phosphate degradation); PWY-5705 (allantoin degradation to glyoxylate III); PWY-5913 (TCA cycle VI (obligate autotrophs)); PWY-5971 (palmitate biosynthesis II (bacteria and plants)); PWY-6125 (superpathway of guanosine nucleotides de novo biosynthesis II); PWY-6353 (purine nucleotides degradation II (aerobic)); PWY-6396 (superpathway of 2,3-butanediol biosynthesis); PWY-6507 (4-deoxy-L-threo-hex-4-enopyranuronate degradation); PWY-6572 (chondroitin sulfate degradation I (bacterial)); PWY-6588 (pyruvate fermentation to acetone); PWY-6608 (guanosine nucleotides degradation III); PWY-6703 (preQ0 biosynthesis); PWY-6891 (thiazole biosynthesis II (Bacillus)); PWY-6895 (superpathway of thiamin diphosphate biosynthesis II); PWY-6901 (superpathway of glucose and xylose degradation); PWY-6969 (TCA cycle V (2-oxoglutarate: ferredoxin oxidoreductase)); PWY-7013 (L-1,2-propanediol degradation); PWY-7199 (pyrimidine deoxyribonucleosides salvage); PWY-7220 (adenosine deoxyribonucleotides de novo biosynthesis II); PWY-7222 (guanosine deoxyribonucleotides de novo biosynthesis II); PWY-7237 (myo-, chiro- and scillo-inositol degradation); PWY-7242 (D-fructuronate degradation); PWY-7328 (superpathway of UDP-glucose-derived O-antigen building blocks biosynthesis); PWY-7456 (mannan degradation); PYRIDOXSYN-PWY (pyridoxal 5′-phosphate biosynthesis I); SALVADEHYPOX-PWY (adenosine nucleotides degradation II); TCA (TCA cycle I (prokaryotic)); THISYN-PWY (superpathway of thiamin diphosphate biosynthesis I); and VALDEG-PWY (L-valine degradation I); wherein the change in metabolic feature relative abundance is indicative of non-CDI causative diarrhea associated with IBD.
Aspect 195) The method of aspect 194, wherein an increase in relative abundance of at least one of: ASPASN-PWY (superpathway of L-aspartate and L-asparagine biosynthesis); DHGLUCONATE-PYR-CAT-PWY (glucose degradation (oxidative)); DTDPRHAMSYN-PWY (dTDP-L-rhamnose biosynthesis I); FASYN-ELONG-PWY (fatty acid elongation-saturated); GALACTARDEG-PWY (D-galactarate degradation I); GLUCARDEG-PWY (D-glucarate degradation I); GLUCARGALACTSUPER-PWY (superpathway of D-glucarate and D-galactarate degradation); GLUCONEO-PWY (gluconcogenesis I); GLUCUROCAT-PWY (superpathway of & beta; -D-glucuronide and D-glucuronate degradation); GLYCOLYSIS (glycolysis I (from glucose 6-phosphate)); GLYCOLYSIS-E-D (superpathway of glycolysis and Entner-Doudoroff); HEMESYN2-PWY (heme biosynthesis II (anacrobic)); HISDEG-PWY (L-histidine degradation I); METHGLYUT-PWY (superpathway of methylglyoxal degradation); P125-PWY (superpathway of (R,R)-butanediol biosynthesis); P162-PWY (L-glutamate degradation V (via hydroxyglutarate)); P163-PWY (L-lysine fermentation to acetate and butanoate); P42-PWY (incomplete reductive TCA cycle); PENTOSE-P-PWY (pentose phosphate pathway); POLYISOPRENSYN-PWY (polyisoprenoid biosynthesis (E. coli)); PWY0-1241 (ADP-L-glycero-& beta; -D-manno-heptose biosynthesis); PWY0-1533 (methylphosphonate degradation I); PWY0-41 (allantoin degradation IV (anaerobic)); PWY0-845 (superpathway of pyridoxal 5′-phosphate biosynthesis and salvage); PWY-5028 (L-histidine degradation II); PWY-5484 (glycolysis II (from fructose 6-phosphate)); PWY-5659 (GDP-mannose biosynthesis); PWY-5695 (urate biosynthesis/inosine 5′-phosphate degradation); PWY-5705 (allantoin degradation to glyoxylate III); PWY-5913 (TCA cycle VI (obligate autotrophs)); PWY-5971 (palmitate biosynthesis II (bacteria and plants)); PWY-6125 (superpathway of guanosine nucleotides de novo biosynthesis II); PWY-6353 (purine nucleotides degradation II (acrobic)); PWY-6396 (superpathway of 2,3-butanediol biosynthesis); PWY-6507 (4-deoxy-L-threo-hex-4-enopyranuronate degradation); PWY-6572 (chondroitin sulfate degradation I (bacterial)); PWY-6588 (pyruvate fermentation to acetone); PWY-6608 (guanosine nucleotides degradation III); PWY-6703 (preQ0 biosynthesis); PWY-6891 (thiazole biosynthesis II (Bacillus)); PWY-6895 (superpathway of thiamin diphosphate biosynthesis II); PWY-6901 (superpathway of glucose and xylose degradation); PWY-6969 (TCA cycle V (2-oxoglutarate: ferredoxin oxidoreductase)); PWY-7013 (L-1,2-propanediol degradation); PWY-7199 (pyrimidine deoxyribonucleosides salvage); PWY-7220 (adenosine deoxyribonucleotides de novo biosynthesis II); PWY-7222 (guanosine deoxyribonucleotides de novo biosynthesis II); PWY-7237 (myo-, chiro- and scillo-inositol degradation); PWY-7242 (D-fructuronate degradation); PWY-7328 (superpathway of UDP-glucose-derived O-antigen building blocks biosynthesis); PWY-7456 (mannan degradation); PYRIDOXSYN-PWY (pyridoxal 5′-phosphate biosynthesis I); SALVADEHYPOX-PWY (adenosine nucleotides degradation II); TCA (TCA cycle I (prokaryotic)); THISYN-PWY (superpathway of thiamin diphosphate biosynthesis I); and VALDEG-PWY (L-valine degradation I), is indicative of non-CDI causative diarrhea associated with IBD.
Aspect 196) The method of any one of aspects 194 or 195, wherein a change in relative abundance of at least four metabolic features is indicative of non-CDI causative diarrhea associated with IBD.
Aspect 197) The method of aspect 188, wherein the characterization of the subject microbiome to determine whether the non-CDI causative diarrhea is associated with IBS or IBD comprises measuring one or more metabolic features from a biological sample from the subject and determining changes in relative abundance, compared to a reference dysbiosis IBS gut microbiome, of at least one of: ASPASN-PWY (superpathway of L-aspartate and L-asparagine biosynthesis); GALACT-GLUCUROCAT-PWY (superpathway of hexuronide and hexuronate degradation); ANAEROFRUCAT-PWY (homolactic fermentation); DHGLUCONATE-PYR-CAT-PWY (glucose degradation (oxidative)); GLUCUROCAT-PWY (superpathway of & beta; -D-glucuronide and D-glucuronate degradation); PWY0-781 (aspartate superpathway); PWY-7242 (D-fructuronate degradation); GOLPDLCAT-PWY (superpathway of glycerol degradation to 1,3-propanediol); PWY-7013 (L-1,2-propanediol degradation); P124-PWY (Bifidobacterium shunt); P122-PWY (heterolactic fermentation); REDCITCYC (TCA cycle VIII (helicobacter)); DENOVOPURINE2-PWY (superpathway of purine nucleotides de novo biosynthesis II); GLUCARGALACTSUPER-PWY (superpathway of D-glucarate and D-galactarate degradation); GALACTARDEG-PWY (D-galactarate degradation I); PWY-6891 (thiazole biosynthesis II (Bacillus)); FUC-RHAMCAT-PWY (superpathway of fucose and rhamnose degradation); PWY-6895 (superpathway of thiamin diphosphate biosynthesis II); PWY0-41 (allantoin degradation IV (anacrobic)); VALDEG-PWY (L-valine degradation I); PWY-7187 (pyrimidine deoxyribonucleotides de novo biosynthesis II); PWY0-1533 (methylphosphonate degradation I); GLUCARDEG-PWY (D-glucarate degradation I); TYRFUMCAT-PWY (L-tyrosine degradation I); PWY-7431 (aromatic biogenic amine degradation (bacteria)); PWY-7094 (fatty acid salvage); and LEU-DEG2-PWY (L-leucine degradation I); wherein the change in metabolic feature relative abundance is indicative of non-CDI causative diarrhea associated with IBD.
Aspect 198) The method of aspect 197, wherein an increase in relative abundance of at least one of: ASPASN-PWY (superpathway of L-aspartate and L-asparagine biosynthesis); GALACT-GLUCUROCAT-PWY (superpathway of hexuronide and hexuronate degradation); ANAEROFRUCAT-PWY (homolactic fermentation); DHGLUCONATE-PYR-CAT-PWY (glucose degradation (oxidative)); GLUCUROCAT-PWY (superpathway of & beta; -D-glucuronide and D-glucuronate degradation); PWY0-781 (aspartate superpathway); PWY-7242 (D-fructuronate degradation); GOLPDLCAT-PWY (superpathway of glycerol degradation to 1,3-propanediol); PWY-7013 (L-1,2-propanediol degradation); P124-PWY (Bifidobacterium shunt); P122-PWY (heterolactic fermentation); REDCITCYC (TCA cycle VIII (helicobacter)); DENOVOPURINE2-PWY (superpathway of purine nucleotides de novo biosynthesis II); GLUCARGALACTSUPER-PWY (superpathway of D-glucarate and D-galactarate degradation); GALACTARDEG-PWY (D-galactarate degradation I); PWY-6891 (thiazole biosynthesis II (Bacillus)); FUC-RHAMCAT-PWY (superpathway of fucose and rhamnose degradation); PWY-6895 (superpathway of thiamin diphosphate biosynthesis II); PWY0-41 (allantoin degradation IV (anaerobic)); VALDEG-PWY (L-valine degradation I); PWY-7187 (pyrimidine deoxyribonucleotides de novo biosynthesis II); PWY0-1533 (methylphosphonate degradation I); and GLUCARDEG-PWY (D-glucarate degradation I), is indicative of non-CDI causative diarrhea associated with IBD.
Aspect 199) The method of aspect 197, wherein a decrease in relative abundance of at least one of: TYRFUMCAT-PWY (L-tyrosine degradation I); PWY-7431 (aromatic biogenic amine degradation (bacteria)); PWY-7094 (fatty acid salvage); and LEU-DEG2-PWY (L-leucine degradation I), is indicative of non-CDI causative diarrhea associated with IBD.
Aspect 200) The method of any one of aspects 197-199, wherein a change in relative abundance of at least four metabolic features is indicative of non-CDI causative diarrhea associated with IBD.
Aspect 201) The method of aspect 189, wherein the characterization of the subject microbiome to determine whether the non-CDI causative diarrhea is associated with IBD UC or IBD CD comprises measuring one or more metabolic features from a biological sample from the subject and determining changes in relative abundance, compared to a reference healthy gut microbiome, of at least one of: PWY-5484 (glycolysis II (from fructose 6-phosphate)); PWY-7220 (adenosine deoxyribonucleotides de novo biosynthesis II); DTDPRHAMSYN-PWY (dTDP-L-rhamnose biosynthesis I); GLUCONEO-PWY (gluconcogenesis I); PWY-7222 (guanosine deoxyribonucleotides de novo biosynthesis II); GLYCOLYSIS (glycolysis I (from glucose 6-phosphate)); POLYISOPRENSYN-PWY (polyisoprenoid biosynthesis (E. coli)); PWY-6969 (TCA cycle V (2-oxoglutarate: ferredoxin oxidoreductase)); PENTOSE-P-PWY (pentose phosphate pathway); PWY-5971 (palmitate biosynthesis II (bacteria and plants)); PWY-7199 (pyrimidine deoxyribonucleosides salvage); PWY-6901 (superpathway of glucose and xylose degradation); PWY-5659 9 (GDP-mannose biosynthesis); PWY-5695 (urate biosynthesis/inosine 5′-phosphate degradation); PWY-7456 (mannan degradation); VALDEG-PWY (L-valine degradation I); ASPASN-PWY (superpathway of L-aspartate and L-asparagine biosynthesis); TCA (TCA cycle I (prokaryotic)); FASYN-ELONG-PWY (fatty acid elongation-saturated); PWY-6703 (preQ0 biosynthesis); HEMESYN2-PWY (heme biosynthesis II (anaerobic)); P42-PWY (incomplete reductive TCA cycle); PWY-5913 (TCA cycle VI (obligate autotrophs)); P162-PWY (L-glutamate degradation V (via hydroxyglutarate)); PWY0-845 (superpathway of pyridoxal 5′-phosphate biosynthesis and salvage); P163-PWY (L-lysine fermentation to acetate and butanoate); THISYN-PWY (superpathway of thiamin diphosphate biosynthesis I); PWY-7242 (D-fructuronate degradation); PWY-6125 (superpathway of guanosine nucleotides de novo biosynthesis II); PWY-6572 (chondroitin sulfate degradation I (bacterial)); and PWY-6507 (4-deoxy-L-threo-hex-4-enopyranuronate degradation); wherein the change in metabolic feature relative abundance is indicative of non-CDI causative diarrhea associated with IBD UC.
Aspect 202) The method of aspect 201, wherein an increase in relative abundance of at least one of: PWY-5484 (glycolysis II (from fructose 6-phosphate)); PWY-7220 (adenosine deoxyribonucleotides de novo biosynthesis II); DTDPRHAMSYN-PWY (dTDP-L-rhamnose I); PWY-7222 (guanosine biosynthesis I); GLUCONEO-PWY (gluconcogenesis deoxyribonucleotides de novo biosynthesis II); GLYCOLYSIS (glycolysis I (from glucose 6-phosphate)); POLYISOPRENSYN-PWY (polyisoprenoid biosynthesis (E. coli)); PWY-6969 (TCA cycle V (2-oxoglutarate: ferredoxin oxidoreductase)); PENTOSE-P-PWY (pentose phosphate pathway); PWY-5971 (palmitate biosynthesis II (bacteria and plants)); PWY-7199 (pyrimidine deoxyribonucleosides salvage); PWY-6901 (superpathway of glucose and xylose degradation); PWY-5659 (GDP-mannose biosynthesis); PWY-5695 (urate biosynthesis/inosine 5′-phosphate degradation); PWY-7456 (mannan degradation); VALDEG-PWY (L-valine degradation I); ASPASN-PWY (superpathway of L-aspartate and L-asparagine biosynthesis); TCA (TCA cycle I (prokaryotic)); FASYN-ELONG-PWY (fatty acid elongation-saturated); PWY-6703 (preQ0 biosynthesis); HEMESYN2-PWY (heme biosynthesis II (anaerobic)); P42-PWY (incomplete reductive TCA cycle); PWY-5913 (TCA cycle VI (obligate autotrophs)); P162-PWY (L-glutamate degradation V (via hydroxyglutarate)); PWY0-845 (superpathway of pyridoxal 5′-phosphate biosynthesis and salvage); P163-PWY (L-lysine fermentation to acetate and butanoate); THISYN-PWY (superpathway of thiamin diphosphate biosynthesis I); PWY-7242 (D-fructuronate degradation); PWY-6125 (superpathway of guanosine nucleotides de novo biosynthesis II); PWY-6572 (chondroitin sulfate degradation I (bacterial)); and PWY-6507 (4-deoxy-L-threo-hex-4-enopyranuronate degradation), is indicative of non-CDI causative diarrhea associated with IBD UC.
Aspect 203) The method of any one of aspects 200-202, wherein a change in relative abundance of at least four metabolic features is indicative of non-CDI causative diarrhea associated with IBD UC.
Aspect 204) The method of aspect 189, wherein the characterization of the subject microbiome to determine whether the non-CDI causative diarrhea is associated with IBD UC or IBD CD comprises measuring one or more metabolic features from a biological sample from the subject and determining changes in relative abundance, compared to a reference healthy gut microbiome, of at least one of: HEMESYN2-PWY (heme biosynthesis II (anaerobic)); PWY-6608 (guanosine nucleotides degradation III); SALVADEHYPOX-PWY (adenosine nucleotides degradation II); GALACTARDEG-PWY (D-galactarate degradation I); PWY-7013 (L-1,2-propanediol degradation); GLUCARGALACTSUPER-PWY (superpathway of D-glucarate and D-galactarate degradation); PWY-6353 (purine nucleotides degradation II (acrobic)); P125-PWY (superpathway of (R,R)-butanediol biosynthesis); GLUCUROCAT-PWY (superpathway of & beta; -D-glucuronide and D-glucuronate degradation); GLUCARDEG-PWY (D-glucarate degradation I); PENTOSE-P-PWY (pentose phosphate pathway); PWY-6891 (thiazole biosynthesis II (Bacillus)); HISDEG-PWY (L-histidine degradation I); PWY0-845 (superpathway of pyridoxal 5′-phosphate biosynthesis and salvage); PWY-5028 (L-histidine degradation II); PWY-7328 (superpathway of UDP-glucose-derived O-antigen building blocks biosynthesis); PWY-7237 (myo-, chiro- and scillo-inositol degradation); P162-PWY (L-glutamate degradation V (via hydroxyglutarate)); PWY-6588 (pyruvate fermentation to acetone); PWY0-1533 (methylphosphonate degradation I); PWY-6396 (superpathway of 2,3-butanediol biosynthesis); PYRIDOXSYN-PWY (pyridoxal 5′-phosphate biosynthesis I); PWY-6895 (superpathway of thiamin diphosphate biosynthesis II); PWY0-1241 (ADP-L-glycero-& beta; -D-manno-heptose biosynthesis); PWY0-41 (allantoin degradation IV (anaerobic)); DHGLUCONATE-PYR-CAT-PWY (glucose degradation (oxidative)); PWY-5705 (allantoin degradation to glyoxylate III); GLYCOLYSIS-E-D (superpathway of glycolysis and Entner-Doudoroff); and METHGLYUT-PWY (superpathway of methylglyoxal degradation); wherein the change in metabolic feature relative abundance is indicative of non-CDI causative diarrhea associated with IBD CD.
Aspect 205) The method of aspect 204, wherein an increase in relative abundance of at least one of: HEMESYN2-PWY (heme biosynthesis II (anaerobic)); PWY-6608 (guanosine nucleotides degradation III); SALVADEHYPOX-PWY (adenosine nucleotides degradation II); GALACTARDEG-PWY (D-galactarate degradation I); PWY-7013 (L-1,2-propanediol degradation); GLUCARGALACTSUPER-PWY (superpathway of D-glucarate and D-galactarate degradation); PWY-6353 (purine nucleotides degradation II (aerobic)); P125-PWY (superpathway of (R,R)-butanediol biosynthesis); GLUCUROCAT-PWY (superpathway of & beta; -D-glucuronide and D-glucuronate degradation); GLUCARDEG-PWY (D-glucarate degradation I); PENTOSE-P-PWY (pentose phosphate pathway); PWY-6891 (thiazole biosynthesis II (Bacillus)); HISDEG-PWY (L-histidine degradation I); PWY0-845 (superpathway of pyridoxal 5′-phosphate biosynthesis and salvage); PWY-5028 (L-histidine degradation II); PWY-7328 (superpathway of UDP-glucose-derived O-antigen building blocks biosynthesis); PWY-7237 (myo-, chiro- and scillo-inositol degradation); P162-PWY (L-glutamate degradation V (via hydroxyglutarate)); PWY-6588 (pyruvate fermentation to acetone); PWY0-1533 (methylphosphonate degradation I); PWY-6396 (superpathway of 2,3-butanediol biosynthesis); PYRIDOXSYN-PWY (pyridoxal 5′-phosphate biosynthesis I); PWY-6895 (superpathway of thiamin diphosphate biosynthesis II); PWY0-1241 (ADP-L-glycero-& beta; -D-manno-heptose biosynthesis); PWY0-41 (allantoin degradation IV (anacrobic)); DHGLUCONATE-PYR-CAT-PWY (glucose degradation (oxidative)); PWY-5705 (allantoin degradation to glyoxylate III); GLYCOLYSIS-E-D (superpathway of glycolysis and Entner-Doudoroff); and METHGLYUT-PWY (superpathway of methylglyoxal degradation), is indicative of non-CDI causative diarrhea associated with IBD CD.
Aspect 206) The method of aspect 204 or 205, wherein a change in relative abundance of at least four metabolic features is indicative of non-CDI causative diarrhea associated with IBD CD.
Aspect 207) The method of aspect 189, wherein the characterization of the subject microbiome to determine whether the non-CDI causative diarrhea is associated with IBD UC or IBD CD comprises measuring one or more metabolic features from a biological sample from the subject and determining changes in relative abundance, compared to a reference dysbiosis IBD UC gut microbiome, of at least one of: ECASYN-PWY (enterobacterial common antigen biosynthesis); PPGPPMET-PWY (ppGpp biosynthesis); ORNARGDEG-PWY (superpathway of L-arginine and L-ornithine degradation); AST-PWY (L-arginine degradation II (AST pathway)); ARGDEG-PWY (superpathway of L-arginine, putrescine, and 4-aminobutanoate degradation); ORNDEG-PWY (superpathway of ornithine degradation); PWY0-1338 (polymyxin resistance); PWY-5028 (L-histidine degradation II); AEROBACTINSYN-PWY (acrobactin biosynthesis); PWY-6071 (superpathway of phenylethylamine degradation); PWY0-321 (phenylacetate degradation I (aerobic)); PWY0-1533 (methylphosphonate degradation I); P241-PWY (coenzyme B biosynthesis); PWY-6148 (tetrahydromethanopterin biosynthesis); PWY-6349 (CDP-archacol biosynthesis); PWY-6654 (phosphopantothenate biosynthesis III); METHANOGENESIS-PWY (methanogenesis from H2 and CO2); PWY-7286 (7-(3-amino-3-carboxypropyl)-wyosine biosynthesis); PWY-5198 (factor 420 biosynthesis); PWY-6167 (flavin biosynthesis II (archaca)); PWY-6641 (superpathway of sulfolactate degradation); PWY-6350 (archactidylinositol biosynthesis); PWY-6141 (archactidylserine and archaetidylethanolamine biosynthesis); PWY-6174 (mevalonate pathway II (archaca)); P261-PWY (coenzyme M biosynthesis I); and PWY-7391 (isoprene biosynthesis II (engineered)); wherein the change in metabolic feature relative abundance is indicative of non-CDI causative diarrhea associated with IBD CD.
Aspect 208) The method of aspect 207, wherein an increase in relative abundance of at least one of: ECASYN-PWY (enterobacterial common antigen biosynthesis); PPGPPMET-PWY (ppGpp biosynthesis); ORNARGDEG-PWY (superpathway of L-arginine and L-ornithinc degradation); AST-PWY (L-arginine degradation II (AST pathway)); ARGDEG-PWY (superpathway of L-arginine, putrescine, and 4-aminobutanoate degradation); ORNDEG-PWY (superpathway of ornithine degradation); PWY0-1338 (polymyxin resistance); PWY-5028 (L-histidine degradation II); AEROBACTINSYN-PWY (acrobactin biosynthesis); PWY-6071 (superpathway of phenylethylamine degradation); PWY0-321 (phenylacetate degradation I (acrobic)); and PWY0-1533 (methylphosphonate degradation I), is indicative of non-CDI causative diarrhea associated with IBD CD.
Aspect 209) The method of aspect 207, wherein a decrease in relative abundance of at least one of: P241-PWY (coenzyme B biosynthesis); PWY-6148 (tetrahydromethanopterin biosynthesis); PWY-6349 (CDP-archacol biosynthesis); PWY-6654 (phosphopantothenate biosynthesis III); METHANOGENESIS-PWY (methanogenesis from H2 and CO2); PWY-7286 (7-(3-amino-3-carboxypropyl)-wyosine biosynthesis); PWY-5198 (factor 420 biosynthesis); PWY-6167 (flavin biosynthesis II (archaca)); PWY-6641 (superpathway of sulfolactate degradation); PWY-6350 (archaetidylinositol biosynthesis); PWY-6141 (archaetidylserine and archactidylethanolamine biosynthesis); PWY-6174 (mevalonate pathway II (archaca)); P261-PWY (coenzyme M biosynthesis I); and PWY-7391 (isoprene biosynthesis II (engineered)), is indicative of non-CDI causative diarrhea associated with IBD CD.
Aspect 210) The method of any one of aspects 207-209, wherein a change in relative abundance of at least four metabolic features is indicative of non-CDI causative diarrhea associated with IBD CD.
Aspect 211) The method of aspect 173, wherein the measuring of one or more metabolic features from a biological sample from the individual comprises determining changes in relative abundance, compared to a reference dysbiosis gut microbiome from individuals with CDI, of at least one of: P221-PWY (octane oxidation); PWY-7374 (1,4-dihydroxy-6-naphthoate biosynthesis I); PWY-6263 (superpathway of menaquinol-8 biosynthesis II); CHLOROPHYLL-SYN (chlorophyllide a biosynthesis I (aerobic, light-dependent)); PWY-5198 (factor 420 biosynthesis); PWY-7286 (7-(3-amino-3-carboxypropyl)-wyosine biosynthesis); PWY-5531 (chlorophyllide a biosynthesis II (anaerobic)); PWY-6349 (CDP-archacol biosynthesis); PWY-5088 (L-glutamate degradation VIII (to propanoate)); PWY-6350 (archactidylinositol biosynthesis); PWY-7159 (chlorophyllide a biosynthesis III (aerobic, light independent)); PWY0-1533 (methylphosphonate degradation I); ORNDEG-PWY (superpathway of ornithinc degradation); PWY0-1338 (polymyxin resistance); ORNARGDEG-PWY (superpathway of L-arginine and L-ornithine degradation); ECASYN-PWY (enterobacterial common antigen biosynthesis); ARGDEG-PWY (superpathway of L-arginine, putrescine, and 4-aminobutanoate degradation); GLYCOL-GLYOXDEG-PWY (superpathway of glycol metabolism and degradation); ENTBACSYN-PWY (enterobactin biosynthesis); THREOCAT-PWY (superpathway of L-threonine metabolism); GOLPDLCAT-PWY (superpathway of glycerol degradation to 1,3-propanediol); PWY-6071 (superpathway of phenylethylamine degradation); PWY0-1277 (3-phenylpropanoate and 3-(3-hydroxyphenyl) propanoate degradation); and PWY-6690 (cinnamate and 3-hydroxycinnamate degradation to 2-oxopent-4-enoate); wherein the change in metabolic feature relative abundance is indicative of non-CDI causative diarrhea associated with IBS.
Aspect 212) The method of aspect 211, wherein an increase in relative abundance of at least one of: P221-PWY (octane oxidation); PWY-7374 (1,4-dihydroxy-6-naphthoate biosynthesis I); PWY-6263 (superpathway of menaquinol-8 biosynthesis II); CHLOROPHYLL-SYN (chlorophyllide a biosynthesis I (aerobic, light-dependent)); PWY-5198 (factor 420 biosynthesis); PWY-7286 (7-(3-amino-3-carboxypropyl)-wyosine biosynthesis); PWY-5531 (chlorophyllide a biosynthesis II (anaerobic)); PWY-6349 (CDP-archacol biosynthesis); PWY-5088 (L-glutamate degradation VIII (to propanoate)); PWY-6350 (archactidylinositol biosynthesis); and PWY-7159 (chlorophyllide a biosynthesis III (aerobic, light independent)), is indicative of non-CDI causative diarrhea associated with IBS.
Aspect 213) The method of aspect 211, wherein a decrease in relative abundance of at least one of: PWY0-1533 (methylphosphonate degradation I); ORNDEG-PWY (superpathway of ornithinc degradation); PWY0-1338 (polymyxin resistance); ORNARGDEG-PWY (superpathway of L-arginine and L-ornithine degradation); ECASYN-PWY (enterobacterial common antigen biosynthesis); ARGDEG-PWY (superpathway of L-arginine, putrescine, and 4-aminobutanoate degradation); GLYCOL-GLYOXDEG-PWY (superpathway of glycol metabolism and degradation); ENTBACSYN-PWY (enterobactin biosynthesis); THREOCAT-PWY (superpathway of L-threonine metabolism); GOLPDLCAT-PWY (superpathway of glycerol degradation to 1,3-propanediol); PWY-6071 (superpathway of phenylethylamine degradation); PWY0-1277 (3-phenylpropanoate and 3-(3-hydroxyphenyl) propanoate degradation); and PWY-6690 (cinnamate and 3-hydroxycinnamate degradation to 2-oxopent-4-enoate), is indicative of non-CDI causative diarrhea associated with IBS.
Aspect 214) The method of any one of aspects 211-213, wherein a change in relative abundance of at least four metabolic features is indicative of non-CDI causative diarrhea associated with IBS.
Aspect 215) The method of aspect 173, wherein the measuring of one or more metabolic features from a biological sample from the individual comprises determining changes in relative abundance, compared to a reference dysbiosis gut microbiome from individuals with CDI, of at least one of the pathways identified in Table 3 as being upregulated greater than 5 fold; wherein the change in metabolic feature relative abundance is indicative of non-CDI causative diarrhea associated with IBD.
Aspect 216) The method of aspect 215, wherein an increase in relative abundance of at least one of: PWY-6151 (S-adenosyl-L-methionine cycle I); P221-PWY (octane oxidation); PWY-5505 (L-glutamate and L-glutamine biosynthesis); PWY-5121 (superpathway of geranylgeranyl diphosphate biosynthesis II (via MEP)); GLYCOGENSYNTH-PWY (glycogen biosynthesis I (from ADP-D-Glucose)); NONMEVIPP-PWY (methylerythritol phosphate pathway I); PWY-6269 (adenosylcobalamin salvage from cobinamide II); PYRIDNUCSYN-PWY (NAD biosynthesis I (from aspartate)); COA-PWY (coenzyme A biosynthesis I); PWY-5686 (UMP biosynthesis); PWY-7560 (methylerythritol phosphate pathway II); PWY-6386 (UDP-N-acetylmuramoyl-pentapeptide biosynthesis II (lysine-containing)); PWY-6277 (superpathway of 5-aminoimidazole ribonucleotide biosynthesis); PWY-5509 (adenosylcobalamin biosynthesis from cobyrinate a,c-diamide I); PWY-7219 (adenosine ribonucleotides de novo biosynthesis); COBALSYN-PWY (adenosylcobalamin salvage from cobinamide I); PWY-6163 (chorismate biosynthesis from 3-dehydroquinate); PWY-6123 (inosine-5′-phosphate biosynthesis I); GLYCOCAT-PWY (glycogen degradation I (bacterial)); ARO-PWY (chorismate biosynthesis I); PWY-6317 (galactose degradation I (Leloir pathway)); PWY-5097 (L-lysine biosynthesis VI); PWY-6385 (peptidoglycan biosynthesis III (mycobacteria)); COMPLETE-ARO-PWY (superpathway of aromatic amino acid biosynthesis); PWY-6892 (thiazole biosynthesis I (E. coli)); PEPTIDOGLYCANSYN-PWY (peptidoglycan biosynthesis I (meso-diaminopimelate containing)); PWY-6122 (5-aminoimidazole ribonucleotide biosynthesis II); PYRIDNUCSAL-PWY (NAD salvage pathway I); PWY-5667 (CDP-diacylglycerol biosynthesis I); PWY-6387 (UDP-N-acetylmuramoyl-pentapeptide biosynthesis I (meso-diaminopimelate containing)); TRNA-CHARGING-PWY (IRNA charging); PWY-5100 (pyruvate fermentation to acetate and lactate II); PWY-7663 (gondoate biosynthesis (anaerobic)); PWY-5104 (L-isoleucine biosynthesis IV); PWY-5973 (cis-vaccenate biosynthesis); PWY0-1319 (CDP-diacylglycerol biosynthesis II); PWY-7221 (guanosine ribonucleotides de novo biosynthesis); PWY4FS-8 (phosphatidylglycerol biosynthesis II (non-plastidic)); PWY-6737 (starch degradation V); PWY-3001 (superpathway of L-isoleucine biosynthesis I); HISTSYN-PWY (L-histidine biosynthesis); PWY-7208 (superpathway of pyrimidine nucleobases salvage); PHOSLIPSYN-PWY (superpathway of phospholipid biosynthesis I (bacteria)); PWY-6121 (5-aminoimidazole ribonucleotide biosynthesis I); PWY-2942 (L-lysine biosynthesis III); SER-GLYSYN-PWY (superpathway of L-serine and glycine biosynthesis I); AEROBACTINSYN-PWY (acrobactin biosynthesis); PWY-6126 (superpathway of adenosine nucleotides de novo biosynthesis II); DAPLYSINESYN-PWY (L-lysine biosynthesis I); NONOXIPENT-PWY (pentose phosphate pathway (non-oxidative branch)); BRANCHED-CHAIN-AA-SYN-PWY (superpathway of branched amino acid biosynthesis); PWY-7229 (superpathway of adenosine nucleotides de novo biosynthesis I); ARGSYNBSUB-PWY (L-arginine biosynthesis II (acetyl cycle)); VALSYN-PWY (L-valine biosynthesis); ILEUSYN-PWY (L-isoleucine biosynthesis I (from threonine)); 1CMET2-PWY (N10-formyl-tetrahydrofolate biosynthesis); GALLATE-DEGRADATION-II-PWY (gallate degradation I); 3-HYDROXYPHENYLACETATE-DEGRADATION-PWY (4-hydroxyphenylacetate degradation); METHYLGALLATE-DEGRADATION-PWY (methylgallate degradation); PWY-7400 (L-arginine biosynthesis IV (archaebacteria)); THRESYN-PWY (superpathway of L-threonine biosynthesis); DENOVOPURINE2-PWY (superpathway of purine nucleotides de novo biosynthesis II); ARGSYN-PWY (L-arginine biosynthesis I (via L-ornithine)); PWY-7210 (pyrimidine deoxyribonucleotides biosynthesis from CTP); PWY-7198 (pyrimidine deoxyribonucleotides de novo biosynthesis IV); POLYAMINSYN3-PWY (superpathway of polyamine biosynthesis II); PWY4FS-7 (phosphatidylglycerol biosynthesis I (plastidic)); PWY0-166 (superpathway of pyrimidine deoxyribonucleotides de novo biosynthesis (E. coli)); GALLATE-DEGRADATION-I-PWY (gallate degradation II); CALVIN-PWY (Calvin-Benson-Bassham cycle); PANTOSYN-PWY (pantothenate and coenzyme A biosynthesis I); PWY-5484 (glycolysis II (from fructose 6-phosphate)); PWY-7184 (pyrimidine deoxyribonucleotides de novo biosynthesis I); HEMESYN2-PWY (heme biosynthesis II (anaerobic)); and POLYISOPRENSYN-PWY (polyisoprenoid biosynthesis (E. coli)), is indicative of non-CDI causative diarrhea associated with IBD.
Aspect 217) The method of aspect 215 or 216, wherein a change in relative abundance of at least four metabolic features is indicative of non-CDI causative diarrhea associated with IBD.
Aspect 218) The method of aspect 173, wherein the measuring of one or more metabolic features from a biological sample from the individual comprises determining relative changes in abundance, compared to a reference gut microbiome, in at least one of: AST-PWY (L-arginine degradation II (AST pathway)); ECASYN-PWY (enterobacterial common antigen biosynthesis); THREOCAT-PWY (superpathway of L-threonine metabolism); PPGPPMET-PWY (ppGpp biosynthesis); PWY0-1338 (polymyxin resistance); PWY-6263 (superpathway of menaquinol-8 biosynthesis II); PWY-7371 (1,4-dihydroxy-6-naphthoate biosynthesis II); PWY-7374 (1,4-dihydroxy-6-naphthoate biosynthesis I); P221-PWY (octane oxidation); PWY-6749 (CMP-legionaminate biosynthesis I); PWY-7456 (mannan degradation); NONMEVIPP-PWY (methylerythritol phosphate pathway I); PWY-5097 (L-lysine biosynthesis VI); PWY-5505 (L-glutamate and L-glutamine biosynthesis); PWY-6122 (5-aminoimidazole ribonucleotide biosynthesis II); PWY-7663 (gondoate biosynthesis (anaerobic)); THRESYN-PWY (superpathway of L-threonine biosynthesis); HEMESYN2-PWY (heme biosynthesis II (anacrobic)); PWY-5304 (superpathway of sulfur oxidation (archaca); PWY-6478 (GDP-D-glycero-alpha-D-manno-heptose biosynthesis); PWY-7198 (pyrimidine deoxyribonucleotides de novo biosynthesis IV); and PWY-7210 (pyrimidine deoxyribonucleotides biosynthesis from CTP); wherein increased abundance of at least one of AST-PWY (L-arginine degradation II (AST pathway)); ECASYN-PWY (enterobacterial common antigen biosynthesis); THREOCAT-PWY (superpathway of L-threonine metabolism); PPGPPMET-PWY (ppGpp biosynthesis); and PWY0-1338 (polymyxin resistance) is associated with CDI causative diarrhea and the individual is treated accordingly; wherein increased abundance of at least one of PWY-6263 (superpathway of menaquinol-8 biosynthesis II); PWY-7371 (1,4-dihydroxy-6-naphthoate biosynthesis II); PWY-7374 (1,4-dihydroxy-6-naphthoate biosynthesis I); P221-PWY (octane oxidation); PWY-6749 (CMP-legionaminate biosynthesis I); and PWY-7456 (mannan degradation) is associated with IBD UC causative diarrhea and the individual is treated accordingly; and wherein increased abundance of at least one of NONMEVIPP-PWY (methylerythritol phosphate pathway I); PWY-5097 (L-lysine biosynthesis VI); PWY-5505 (L-glutamate and L-glutamine biosynthesis); PWY-6122 (5-aminoimidazole ribonucleotide biosynthesis II); PWY-7663 (gondoate biosynthesis (anaerobic)); THRESYN-PWY (superpathway of L-threonine biosynthesis); HEMESYN2-PWY (heme biosynthesis II (anaerobic)); PWY-5304 (superpathway of sulfur oxidation (archaca); PWY-6478 (GDP-D-glycero-alpha-D-manno-heptose biosynthesis); PWY-7198 (pyrimidine deoxyribonucleotides de novo biosynthesis IV); and PWY-7210 (pyrimidine deoxyribonucleotides biosynthesis from CTP) is associated with IBD causative diarrhea and the individual is treated accordingly.
Aspect 219) A complex comprising a plurality of oligonucleotide primer sets hybridized to nucleic acid template sequences, wherein the nucleic acid template sequences are taxonomically specific sequences associated with taxonomical features identified in tables 9-17.
Aspect 220) The complex of aspect 219, wherein at least 5 or at least 10 oligonucleotide primer sets are hybridized to nucleic acid template sequences.
Aspect 221) A kit for measuring for presence or absence or a certain level of one or more taxonomical feature(s) from a biological sample from an individual, comprising: (a) a plurality of sets of oligonucleotide primers, wherein each set of primers hybridize to a different nucleic acid template sequence for amplying taxonomically specific sequences; and (b) a polymerase enzyme; wherein the individual sets of oligonucleotide primers hybridize to a taxonomically specific sequence associated with the taxonomical features identified in tables 9-17.
Aspect 222) The kit according to aspect 221, wherein the master mix further comprises deoxynucleoside triphosphates; and at least one indicator for detecting an amplification product by a change in color or fluorescence.
Aspect 223) The kit according to aspect 222, wherein the deoxynucleoside triphosphates comprise dTTP, dGTP, dATP, dCTP and/or dUTP.
Aspect 224) The kit according to aspect 221, comprising at least 5, at least 10, at least 20, at least 40, at least 60, at least 80, at least 100, at least 120, at least 140, at least 160, at least 180, or at least 200 individual sets of oligonucleotide primers.
Aspect 225) The kit according to aspect 221, wherein the individual sets of oligonucleotide primers are bound to a support substrate.
Aspect 226) A kit for measuring for presence or absence or a certain level of one or more biochemical feature(s) from a biological sample from an individual, comprising: (a) a plurality of wells, wherein at least one well comprises one or more reagents suitable for colorimetric mediated analysis of levels of a metabolite; and (b) a guide for interpreting the colorimetric results; wherein the individual wells comprise reagents suitable for measurement of metabolites associated with the metabolic pathways identified in tables 1-8.
Aspect 227) The kit according to aspect 226, comprising at least 5, at least 10, at least 20, at least 40, at least 60, at least 80, at least 100, at least 120, at least 140, at least 160, at least 180, or at least 200 individual wells comprising reagents suitable for colorimetric mediated analysis of levels of a metabolite.
Aspect 228) A composition for treatment of CDI associated microbiome dysbiosis, comprising agents that decrease the activity of at least one metabolic pathway, wherein the metabolic pathway is AST-PWY (L-arginine degradation II (AST pathway)), ECASYN-PWY (enterobacterial common antigen biosynthesis), THREOCAT-PWY (superpathway of L-threonine metabolism), PPGPPMET-PWY (ppGpp biosynthesis), and/or PWY0-1338 (polymyxin resistance).
Aspect 229) A composition for treatment of IBD UC associated microbiome dysbiosis, comprising agents that decrease the activity of at least one metabolic pathway, wherein the metabolic pathway is PWY-6263 (superpathway of menaquinol-8 biosynthesis II), PWY-7371 (1,4-dihydroxy-6-naphthoate biosynthesis II), PWY-7374 (1,4-dihydroxy-6-naphthoate biosynthesis I), P221-PWY (octane oxidation), PWY-6749 (CMP-legionaminate biosynthesis I), and/or PWY-7456 (mannan degradation).
Aspect 230) A composition for treatment of IBD associated microbiome dysbiosis, comprising agents that decrease the activity of at least one metabolic pathway, wherein the metabolic pathway is NONMEVIPP-PWY (methylerythritol phosphate pathway I), PWY-5097 (L-lysine biosynthesis VI), PWY-5505 (L-glutamate and L-glutamine biosynthesis), PWY-6122 (5-aminoimidazole ribonucleotide biosynthesis II), PWY-7663 (gondoate biosynthesis (anacrobic)), THRESYN-PWY (superpathway of L-threonine biosynthesis), HEMESYN2-PWY (heme biosynthesis II (anaerobic)), PWY-5304 (superpathway of sulfur oxidation (archaca)), PWY-6478 (GDP-D-glycero-alpha-D-manno-heptose biosynthesis), PWY-7198 (pyrimidine deoxyribonucleotides de novo biosynthesis IV), and/or PWY-7210 (pyrimidine deoxyribonucleotides biosynthesis from CTP).
Aspect 231) The composition of any one of aspects 228-230, wherein the agent is an antibiotic, antimicrobial, antiviral, small molecule, peptide, amino acid, carbohydrate, sugar, fat, metabolite, oligonucleotide, microbe(s), or any combination thereof.
Aspect 232) The composition of aspect 231, wherein the composition is formulated for oral delivery, intravenous delivery, or delivery as a suppository.
Aspect 233) A method of decreasing the rate of CDI associated health care facility epidemics, comprising testing individuals for CDI associated taxonomic features and/or metabolic features as described in any one of tables 1-17, and isolating the individual if they have taxonomic features and/or metabolic features indicative of CDI.
Aspect 234) A method of identifying features suitable for methods of disease diagnosis and/or treatment regimen prescription comprising, identifying and selecting classifiers using microbiome data generated from two or more different 16S sequencing strategies and/or two or more different populations.
Aspect 235) The method of aspect 234, wherein the Taxa4Meta data analysis pipeline is used to identify the features.
Aspect 236) The method of aspect 234 or 235, wherein technical and/or demographic bias is reduced when compared to analysis performed with less than two 16S sequencing strategies and/or different populations.

### EXAMPLES

The following examples are included to demonstrate preferred embodiments of the disclosure. It should be appreciated by those of skill in the art that the techniques disclosed in the examples that follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Materials and Methods

Simulation of full-length and region-specific 16S amplicon data: two reference databases—NCBI 16S rRNA RefSeq database (downloaded in July 2019) and Ribosomal Database Project (RDP) database (release 11.5)²⁸ were downloaded for data simulation. The cutadapt (version 2.4)²⁹ was then used to extract sequence fragments as full-length amplicons of targeted 16S variable regions (V1-V3. V3-V5. V4 and V6-V9) based on the forward and reverse primers listed in Table 22; an error rate of 0.2 was permitted during sequence extraction. Further sequence length trimming and random simulation of sequence abundance and quality score were performed for specific benchmarking purposes as indicated below.

Benchmarking of sequence clustering and denoising using simulated amplicons with variable length: for benchmarking the accuracy of clustering or denoising for amplicon data using variable sequence lengths, random count ranging from 1 to 50 was assigned for each parent full-length amplicon extracted from NCBI 16S rRNA RefSeq sequences. Since traditional 454 data is normally generated from reverse orientation, length trimming from either forward or reverse orientation was applied to each type of amplicon data resulting in 100, 150, 170, 200, 250, 300, 350, 400 and 450 bases for V1-V3, V3-V5 and V6-V9 amplicon data and 100, 150, 170, 200 and 250 bases for V4 amplicon data. Random phred quality score (ASCII_BASE=33) ranging from 30 to 42 was assigned to each base for sequencing denoising. Simulated amplicons of each sequence length represents one sample. All samples with the same sequence orientation from the same 16S region were then included for closed-reference or de novo clustering using UCLUST (v1.2.22)³⁰ or VSEARCH (v2.9)³¹ or denoising using DADA2 (v1.8)³². Sequence similarity thresholds including 0.97, 0.99 and 1.00 were evaluated for each clustering strategy. The comprehensive SILVA database (release 132) was used for closed-reference OTU picking. Because simulated amplicons of variable length originating from the same parent full-length amplicon had the same sequence counts, pairwise Spearman correlation analysis was performed for sequence counts of any two sequence lengths (as two independent samples) in the OTU count tables.

Benchmarking of taxonomic over-classification: taxonomic over-classification for short amplicon data represents an important criteria for controlling false positives. Using the default parameters in the Bayesian-based Lowest Common Ancestor (BLCA) tool¹¹ and its default database of NCBI 16S rRNA RefSeq was used to annotate random and repeat sequences that were previously generated for benchmarking IDTAXA and other annotation tools¹⁰. Full-length 16S amplicons of unannotated sequences (at least down to family rank; 868.902 sequences) extracted from RDP database (release 11.5) was further used for testing BLCA. BLASTN search of unannotated sequences against NCBI 16S rRNA RefSeq database also confirmed that no best hits were identified at 97% threshold applied to both sequence identity and coverage. Simulated amplicons of unannotated RDP sequences were tested using different thresholds of sequence coverage and identity ranging from 0.85 to 1.00 in BLCA. Ten iterations of random sub-sampling (1%) and BLCA annotation on those unannotated amplicons were performed for statistical determination of optimal sequence coverage and identity required for BLCA. Taxonomic over-classification rate is defined as the classifiable proportion of unannotated amplicons at species level. The confidence score of taxonomic assignment was not considered at this stage.

Benchmarking of taxonomic accuracy using simulated amplicons of variable length: for benchmarking taxonomic accuracy of BLCA, simulated amplicons of variable length were generated by trimming full-length amplicons of NCBI 16S RefSeq from either forward or reverse orientation resulting in 100, 150, 170, 200, 250, 300, 350, 400 and 450 bases for V1-V3, V3-V5 and V6-V9 amplicon data, and 100, 150, 170, 200 and 250 bases for V4 amplicon data. In addition to the known taxonomic lineage, the parent 16S sequences of simulated amplicons are also present in the BLCA default reference database using NCBI 16S RefSeq, thus taxonomic misclassification could be evaluated. Misclassification rate is defined as the proportion of incorrect annotations for simulated amplicons. To further determine the optimal confidence threshold of BLCA for mitigating misclassification, amplicons with a selected sequence length range were combined to calculate the proportion of correct versus incorrect annotations using defined thresholds. Given the already known taxonomic lineage, true positive (TP) and false negative (FN) hits are correct annotations, whereas true negative (TN) and false positive (FP) hits are incorrect annotations.

Design of the Taxa4Meta pipeline: based on the results of the inventors comprehensive benchmarking, the inventors constructed a new computational pipeline “Taxa4Meta” for analyzing 16S amplicon data with an optimal range of variable sequence lengths. This pipeline implements several open-source programs including VSEARCH³¹ for stringent clustering (99% identity) optimized for 16S amplicon data with the selected variable lengths after quality trimming. BLCA¹¹ with optimal region-specific confidence thresholds for stringent species annotation of OTUs. and IDTAXA¹⁰ for annotating OTUs that could not be annotated down to species resolution. Collapsed taxonomic profiles from OTU tables were used for downstream analyses during 16S meta-analysis.

Benchmarking of taxonomic profiling accuracy using Taxa4Meta versus other 16S pipelines: to test the feasibility and accuracy of different 16S pipelines using the simulated and experimental datasets^{12,21} for data processing and is designed to retain reads for accurate sequence clustering and improved taxonomic accuracy. Simulated datasets were prepared from NCBI 16S RefSeq as indicated above: full-length amplicons of V1-V3, V3-V5, V4 and V6-V9 were simulated, random sequence count ranging from 1 to 50 and random phred quality score (ASCII_BASE=33) ranging from 30 to 42 were generated for each full-length amplicon. Further length trimming was performed for each full-length amplicons: V1-V3 forward amplicons (200, 250, 300, 350, 400 and 450 bases), V1-V3 reverse amplicons (300, 350, 400 and 450 bases), V3-V5 forward amplicons (250, 300, 350, 400 and 450 bases), V3-V5 reverse amplicons (300, 350, 400 and 450 bases), both forward and reverse amplicons of V4 (200 and 250 bases), V6-V9 forward amplicons (300, 350, 400 and 450 bases), V6-V9 reverse amplicons (250, 300, 350, 400 and 450 bases). Trimmed amplicons from the same sequence orientation of the same 16S variable region were combined for benchmarking different 16S pipelines. NCBI 16S taxonomic lineage of NCBI 16S RefSeq was used as the ground truth (reference annotations) for comparison.

A Korean stool microbiome dataset¹² with the same DNA extracts used for 454 V1-V4, Illumina V1-V3, Illumina V3-V4, Illumina V4, and Illumina shotgun metagenomic sequencing was used as the real human microbiome dataset for benchmarking different 16S pipelines. Primers retained in the sequence reads were removed by positional trimming. Illumina paired-end reads were merged using USEARCH (version 8.1.1831) with default parameters prior to benchmarking 16S pipelines.

Key 16S pipelines including DADA2-IDTAXA, DADA2-RDP, UCLUST-UCLUST, USEARCH-RDP, Taxa4Meta, Kraken2 and MetaPhlAn2 were benchmarked with the simulated amplicons and Korean human microbiome dataset. Specifically, the analysis procedure for each pipeline were described below:

DADA2-IDTAXA pipeline. DADA2 (version 1.8) was used for denoising amplicon data after quality filtering with maximum expected error of 2 and minimum length of 200 bases. IDTAXA together with its pre-built RDP training set (version 16) was used for taxonomic annotation (could only down to genus level) with the confidence threshold of 70 using 100 bootstraps.

DADA2-RDP pipeline. DADA2 (version 1.8) was used for denoising amplicon data after quality filtering with maximum expected error of 2 and minimum length of 200 bases. RDP Naive Bayesian Classifier algorithm implemented in DADA2's assignTaxonomy function together with its pre-formatted RDP training set (version 16) was used for taxonomic annotation (could down to species level) using minimum bootstrap confidence of 50.

UCLUST-UCLUST pipeline. UCLUST (version 1.2.22q) was used for clustering amplicon data with 97% sequence similarity after quality filtering with the minimum quality threshold of 20 and the minimum length of 140 bases. Representative sequence of OTUs were selected with pick_rep_set.py script with default parameters. UCLUST implemented in assign_taxonomy.py script together with SILVA database (release 123; choice of silva_132_97_16S.fna) was used for taxonomic annotation, which could be down to species level using minimum bootstrap confidence of 0.5. All procedures were completed in QIIME platform (version 1.9.1). This pipeline is similar to the meta-analysis method used by Mancabelli et al.²²

USEARCH-RDP pipeline. USEARCH was used for clustering amplicon data with 100% sequence similarity after quality filtering with maximum expected error of 2 and minimum length of 200 bases. RDP classifier (version 2.12) together with RDP training set (version 16) was used for taxonomic annotation, which could be down to species level using minimum bootstrap confidence of 0.5. This pipeline is similar to the meta-analysis method used by Duvallet et al.²⁰

Taxa4Meta pipeline. Taxa4Meta (version 1.22) was used for clustering amplicon data after quality filtering with maximum expected error of 2 and selected range of variable lengths as suggested by Taxa4Meta itself. Taxonomic annotation by Taxa4Meta binary classifier could be down to species level.

Metagenomic classifiers. Paired-end sequences were trimmed and filtered to meet a maximum expected error of 2 with a minimum read length of 50. Kraken2 (version 2.0.8) with its pre-built database (minikraken2_v2_8 GB_201904_UPDATE) with default parameters was used for taxonomic profiling for shotgun metagenomic data. MetaPhlAn2 (version 2.7.7) with it default database (mpa_v20_m200) with default parameters was used for taxonomic profiling for shotgun metagenomic data. Kraken2 family-level abundance results were used as the reference for comparisons across different 16S pipelines. Given the high precision on species identification. MetaPhlAn2 species-level abundance results were used as the reference for evaluating species calls of different 16S pipelines. A pseudo sample was created by averaging each family-level abundance of all 27 WGS samples, then the abundance-weighted Jaccard distance was calculated between the pseudo sample and any real sample analyzed by different pipelines.

Microbiome meta-analysis of diarrheal microbiome datasets: each diarrheal dataset run on the Taxa4Meta pipeline adopted optimal taxonomic thresholds for each 16S variable region. Taxa4Meta command for each dataset was indicated in Table 21. Relative abundance of collapsed species profiles generated from each Taxa4Meta OTU count table was used without rarefaction but required a minimum 1.000 reads per sample. If species was assigned by Taxa4Meta-BLCA, the taxonomic lineage from NCBI 16S RefSeq was adopted for that species to avoid inconsistency in taxonomic lineage. Merging of Taxa4Meta collapsed species of all datasets was based on taxonomic lineages.

Predictive metagenome functions: PICRUSt2 (see e.g., Holmes et al., Generative models for microbial metagenomics. PLOS One 7, (2012)) with default parameters used Taxa4Meta OTU count tables and OTU sequences to infer metabolic pathway abundance profiles for each dataset. Merging of PICRUSt2 pathway profiles of all datasets was based on MetaCyc pathway IDs. Both LEfSe analysis (one-against-one test mode; version 1.0) and random forest (RF)-based feature ranking (default parameters in Orange version 3.20) were performed using pathway abundance profiles for each disease (CDI/CD/UC/IBS) and Control subjects. Mean decrease accuracy (MDA) score from RF-based analysis was used to rank pathways. Top 20 pathways must be listed by both RF-based feature ranking result and LEfSe analysis result, and was selected for downstream analysis including heatmap generation.

α-Diversity and β-diversity analyses: two α-diversity indices were calculated at OTU level including Shannon index (alpha_diversity.py in QIIME v1.9.1) and richness index (breakaway package version 4.7.5). In QIIME v1.9.1. Principal coordinate analysis (PCoA) with abundance-weighted Jaccard distance metric was applied for β-diversity analysis using combined collapsed species profile. ANOSIM test for group comparison was performed using the beta-diversity distance profile and the permutations of 999.

Fitting factors onto β-diversity ordination plot: fitting factors (taxa) onto a two-dimensional ordination plot (first two coordinates) was performed using the envfit function in vegan package (version 2.5-7). Taxonomic abundance profile at family level was used as factors in this analysis. Significance of fitted factors was established using the permutation of 999 in the envfit run.

Microbiome enterotyping: Microbiome enterotyping was performed with family abundance profiles of all meta-analysis adult training sets. Dirichlet multinomial mixtures (DMM) algorithm, a classical method for clustering community profile data, was used for microbiome enterotyping in this study.

Supervised classification and independent cohort validation: all supervised classification procedures were performed using Orange software³³ (version 3.20) applied to the reported cohorts with clinical definitions. We adopted the original sample grouping information from each cohort, so the gold standard definition is clear for each sample. Unless otherwise stated, random forest-based feature ranking was used as a first pass to select top 100 input features (taxa or pathway) for downstream supervised learning. Unless performing sub-sampling of samples, all input samples was used for training procedure. Supervised classification was performed using individual learning algorithms including Random Forest (RF), Support Vector Machine (SVM), Naïve Bayes (NB) and Neural Network (NN). A Stack model as an aggregated meta-learner of RF, SVM and NB was also assessed. Unless otherwise stated, a 5-fold cross-validation method was applied for sub-sampling of training and test data during the training procedure. Receiver-operating-characteristic (ROC) analysis was performed using the training results. Values of area-under-the curve (AUC) and classification accuracy (CA) were calculated to evaluate the performance of each classification model. CA refers to the proportion of correct predicted samples from the classification model compared to the original clinical diagnosis. Independent validation of classification models was performed using datasets of recently reported microbiome surveys of human diarrheal diseases that were not included in the training set. Each validation dataset was analyzed individually using Taxa4Meta pipeline to generate taxonomic profile data for validating classification models. CDI and IBD scores refer to the predicted scores of each sample as the class of CDI and IBD, respectively.

Statistical analysis: unless otherwise stated, comparisons between two groups were made using non-parametric Mann-Whitney-Wilcoxon two-tailed test, and comparisons for more than two groups were made using non-parametric Kruskal-Wallis two-tailed test. Multiple comparisons and pairwise Spearman or Pearson correlations were adjusted using the Benjamini-Hochberg (BH) false discovery rate (p<0.05, regarded as statistically significant).

Data availability: data accession numbers and reference to publicly available 16S datasets including training and validation datasets are listed in Table 21.

Code availability: the code for Taxa4Meta is available at https://github.com/qinglong89/Taxa4Meta. Benchmarking analyses and codes for amplicon data simulation and analysis, and the scripts of benchmarking analysis of different 16S pipelines can be accessed at https://github.com/qinglong89/Taxa4Meta-ParameterBenchmarking.

### Example 2—Data Tables

TABLE 1
Disease-specific pathway features (metabolic pathway)

| MetaCyc | Pathway | Abundance (median) | | | | | |
|---|---|---|---|---|---|---|---|
| Pathway ID | description | CD | UC | CDI | Ctrl | IBS | Specificity |
| AST-PWY | L-arginine | 163.46 | 22.28 | 504.16 | 13.06 | 12.32 | CDI |
| | degradation II | | | | | | |
| | (AST pathway) | | | | | | |
| ECASYN- | enterobacterial | 343.84 | 41.13 | 726.13 | 25.74 | 12.83 | CDI |
| PWY | common antigen | | | | | | |
| | biosynthesis | | | | | | |
| THREOCAT- | superpathway of | 99.87 | 19.97 | 251.61 | 11.10 | 3.28 | CDI |
| PWY | L-threonine | | | | | | |
| | metabolism | | | | | | |
| PPGPPMET- | ppGpp | 773.66 | 110.34 | 1396.50 | 62.07 | 82.71 | CDI |
| PWY | biosynthesis | | | | | | |
| PWY0-1338 | polymyxin | 217.53 | 37.29 | 516.94 | 17.90 | 11.72 | CDI |
| | resistance | | | | | | |
| PWY-6263 | superpathway of | 223.72 | 2885.87 | 14.98 | 792.45 | 1229.92 | UC |
| | menaquinol-8 | | | | | | |
| | biosynthesis II | | | | | | |
| PWY-7371 | 1,4-dihydroxy-6- | 78.11 | 1246.50 | 5.00 | 312.42 | 470.08 | UC |
| | naphthoate | | | | | | |
| | biosynthesis II | | | | | | |
| PWY-7374 | 1,4-dihydroxy-6- | 31.17 | 731.28 | 0.33 | 199.35 | 269.79 | UC |
| | naphthoate | | | | | | |
| | biosynthesis I | | | | | | |
| P221-PWY | octane oxidation | 945.76 | 2359.46 | 4.00 | 725.62 | 991.05 | UC |
| PWY-6749 | CMP- | 2605.54 | 11572.94 | 443.55 | 3313.21 | 4371.99 | UC |
| | legionaminate | | | | | | |
| | biosynthesis I | | | | | | |
| PWY-7456 | mannan | 11104.22 | 26108.29 | 1456.44 | 4288.02 | 7878.13 | UC |
| | degradation | | | | | | |
| NONMEVIPP- | methylerythritol | 59111.79 | 101543.99 | 9563.19 | 17865.06 | 30036.41 | IBD |
| PWY | phosphate | | | | | | |
| | pathway I | | | | | | |
| PWY-5097 | L-lysine | 68211.98 | 113250.49 | 11456.24 | 20079.19 | 33973.70 | IBD |
| | biosynthesis VI | | | | | | |
| PWY-5505 | L-glutamate and | 45879.23 | 70219.69 | 5003.82 | 15125.30 | 21801.52 | IBD |
| | L-glutamine | | | | | | |
| | biosynthesis | | | | | | |
| PWY-6122 | 5-aminoimidazole | 65716.57 | 105563.32 | 11025.56 | 18871.61 | 31355.19 | IBD |
| | ribonucleotide | | | | | | |
| | biosynthesis II | | | | | | |
| PWY-7663 | gondoate | 78634.26 | 118643.90 | 13238.23 | 20807.55 | 37675.48 | IBD |
| | biosynthesis | | | | | | |
| | (anaerobic) | | | | | | |
| THRESYN- | superpathway of | 65062.46 | 106314.58 | 10222.53 | 18718.10 | 31205.23 | IBD |
| PWY | L-threonine | | | | | | |
| | biosynthesis | | | | | | |
| HEMESYN2- | heme | 6747.68 | 5529.64 | 2549.51 | 794.28 | 1432.83 | IBD |
| PWY | biosynthesis II | | | | | | |
| | (anaerobic) | | | | | | |
| PWY-5304 | superpathway of | 11101.08 | 18412.38 | 552.78 | 3885.00 | 5899.68 | IBD |
| | sulfur oxidation | | | | | | |
| | (archaea) | | | | | | |
| PWY-6478 | GDP-D-glycero- | 5214.73 | 5706.07 | 627.92 | 1111.34 | 1992.51 | IBD |
| | alpha-D-manno- | | | | | | |
| | heptose | | | | | | |
| | biosynthesis | | | | | | |
| PWY-7198 | pyrimidine | 7291.60 | 8216.14 | 437.81 | 2364.29 | 3442.81 | IBD |
| | deoxyribonucleotides | | | | | | |
| | de novo | | | | | | |
| | biosynthesis IV | | | | | | |
| PWY-7210 | pyrimidine | 8669.00 | 10308.98 | 566.65 | 2882.09 | 4152.64 | IBD |
| | deoxyribonucleotides | | | | | | |
| | biosynthesis | | | | | | |
| | from CTP | | | | | | |

CD = Crohn's Disease; UC = Ulcerative Colitis; CDI = Clostridiodes difficile Infection; Ctrl = Control; IBS = Irritable Bowel Syndrome; IBD = Inflammatory Bowel Disease.

TABLE 2
IBD vs IBS delineation features - (metabolic pathway)

| | | | | FC | | | | | | |
|---|---|---|---|---|---|---|---|---|---|---|
| MetaCyc | Pathway | RFFR | | IBD vs | Abundance (IBD) | | | Abundance (IBS) | | |
| Pathway ID | description | # | MDA | IBS | M | SD | DP | M | SD | DP |
| PWY-6876 | isopropanol | 1 | 0.016132 | 1.66 | 723.95 | 3230.75 | 0.89 | 435.82 | 1957.09 | 0.98 |
| | biosynthesis | | | | | | | | | |
| PWY-5100 | pyruvate | 2 | 0.015964 | 4.67 | 135870.24 | 152815.93 | 1.00 | 29089.95 | 17522.28 | 1.00 |
| | fermentation to | | | | | | | | | |
| | acetate and | | | | | | | | | |
| | lactate II | | | | | | | | | |
| PWY-7376 | cob(II)yrinate | 3 | 0.014280 | 1.18 | 67.09 | 1063.06 | 0.36 | 56.93 | 251.97 | 0.54 |
| | a,c-diamide | | | | | | | | | |
| | biosynthesis II | | | | | | | | | |
| | (late cobalt | | | | | | | | | |
| | incorporation) | | | | | | | | | |
| PWY-5507 | adenosylcobalamin | 4 | 0.013715 | 4.85 | 368.53 | 1339.55 | 0.56 | 75.98 | 227.22 | 0.63 |
| | biosynthesis I | | | | | | | | | |
| | (early cobalt | | | | | | | | | |
| | insertion) | | | | | | | | | |
| P381-PWY | adenosylcobalamin | 5 | 0.012953 | 0.64 | 40.46 | 245.62 | 0.36 | 62.75 | 260.44 | 0.53 |
| | biosynthesis II | | | | | | | | | |
| | (late cobalt | | | | | | | | | |
| | incorporation) | | | | | | | | | |
| PWY-5121 | superpathway of | 6 | 0.012701 | 4.54 | 127725.86 | 145558.38 | 1.00 | 28115.14 | 15593.29 | 1.00 |
| | geranylgeranyl | | | | | | | | | |
| | diphosphate | | | | | | | | | |
| | biosynthesis II | | | | | | | | | |
| | (via MEP) | | | | | | | | | |
| PWY-6892 | thiazole | 7 | 0.011727 | 4.96 | 119155.10 | 141683.42 | 1.00 | 24004.43 | 15083.44 | 1.00 |
| | biosynthesis I | | | | | | | | | |
| | (E. coli) | | | | | | | | | |
| ASPASN- | superpathway | 8 | 0.011352 | 5.15 | 124206.27 | 151781.39 | 1.00 | 24106.22 | 15009.33 | 1.00 |
| PWY | of L-aspartate | | | | | | | | | |
| | and L- | | | | | | | | | |
| | asparagine | | | | | | | | | |
| | biosynthesis | | | | | | | | | |
| TYRFUMCAT- | L-tyrosine | 9 | 0.011017 | 0.02 | 153.79 | 1444.97 | 0.64 | 8292.04 | 25541.88 | 0.71 |
| PWY | degradation I | | | | | | | | | |
| PWY-6122 | 5-aminoimidazole | 10 | 0.010214 | 3.94 | 138322.07 | 155913.44 | 1.00 | 35079.61 | 17750.87 | 1.00 |
| | ribonucleotide | | | | | | | | | |
| | biosynthesis II | | | | | | | | | |
| PWY-7431 | aromatic | 11 | 0.009966 | 0.14 | 125.58 | 1193.86 | 0.90 | 887.36 | 3722.68 | 0.97 |
| | biogenic amine | | | | | | | | | |
| | degradation | | | | | | | | | |
| | (bacteria) | | | | | | | | | |
| PWY-7094 | fatty acid | 12 | 0.009691 | 0.09 | 833.48 | 3751.14 | 0.70 | 9353.82 | 28181.45 | 0.76 |
| | salvage | | | | | | | | | |
| PWY-3781 | aerobic | 13 | 0.009635 | 0.24 | 4627.12 | 22834.50 | 0.87 | 18914.47 | 55652.73 | 0.97 |
| | respiration I | | | | | | | | | |
| | (cytochrome c) | | | | | | | | | |
| PWY-7221 | guanosine | 14 | 0.009578 | 4.10 | 139893.46 | 161207.20 | 1.00 | 34118.96 | 16764.40 | 1.00 |
| | ribonucleotides | | | | | | | | | |
| | de novo | | | | | | | | | |
| | biosynthesis | | | | | | | | | |
| PWY-6277 | superpathway of | 15 | 0.009356 | 3.94 | 138322.07 | 155913.44 | 1.00 | 35079.61 | 17750.87 | 1.00 |
| | 5-aminoimidazole | | | | | | | | | |
| | ribonucleotide | | | | | | | | | |
| | biosynthesis | | | | | | | | | |
| GALACT- | superpathway | 16 | 0.009320 | 5.47 | 55138.14 | 73119.47 | 1.00 | 10083.39 | 6875.72 | 1.00 |
| GLUCUROCAT- | of hexuronide | | | | | | | | | |
| PWY | and hexuronate | | | | | | | | | |
| | degradation | | | | | | | | | |
| THRESYN- | superpathway | 17 | 0.009171 | 4.07 | 142354.23 | 163498.18 | 1.00 | 34985.56 | 17229.65 | 1.00 |
| PWY | of L-threonine | | | | | | | | | |
| | biosynthesis | | | | | | | | | |
| GLYCOCAT- | glycogen | 18 | 0.008796 | 4.35 | 159288.63 | 184015.95 | 1.00 | 36577.81 | 20297.09 | 1.00 |
| PWY | degradation I | | | | | | | | | |
| | (bacterial) | | | | | | | | | |
| ANAEROFRUCAT- | homolactic | 19 | 0.008761 | 5.05 | 113262.98 | 139978.58 | 1.00 | 22408.10 | 13981.72 | 1.00 |
| PWY | fermentation | | | | | | | | | |
| PWY-6609 | adenine and | 20 | 0.008691 | 4.46 | 121062.47 | 138331.24 | 1.00 | 27163.52 | 16702.31 | 1.00 |
| | adenosine | | | | | | | | | |
| | salvage III | | | | | | | | | |
| DHGLUCONATE- | glucose | 21 | 0.008217 | 18.85 | 55.86 | 1116.50 | 0.25 | 2.96 | 14.02 | 0.38 |
| PYR-CAT- | degradation | | | | | | | | | |
| PWY | (oxidative) | | | | | | | | | |
| LEU-DEG2- | L-leucine | 22 | 0.007897 | 0.08 | 535.70 | 4673.65 | 0.66 | 6338.85 | 19428.76 | 0.71 |
| PWY | degradation I | | | | | | | | | |
| PWY-5097 | L-lysine | 23 | 0.007734 | 3.93 | 147736.56 | 168639.95 | 1.00 | 37580.67 | 18590.79 | 1.00 |
| | biosynthesis VI | | | | | | | | | |
| PWY-6317 | galactose | 24 | 0.007726 | 4.36 | 117717.50 | 139097.49 | 1.00 | 26969.73 | 17589.78 | 1.00 |
| | degradation I | | | | | | | | | |
| | (Leloir pathway) | | | | | | | | | |
| PWY-6386 | UDP-N- | 25 | 0.007528 | 3.91 | 129151.67 | 145723.67 | 1.00 | 33067.64 | 16734.16 | 1.00 |
| | acetylmuramoyl- | | | | | | | | | |
| | pentapeptide | | | | | | | | | |
| | biosynthesis II | | | | | | | | | |
| | (lysine- | | | | | | | | | |
| | containing) | | | | | | | | | |
| P4-PWY | superpathway | 26 | 0.007027 | 4.79 | 58287.69 | 83243.88 | 1.00 | 12169.85 | 8822.67 | 1.00 |
| | of L-lysine, L- | | | | | | | | | |
| | threonine and | | | | | | | | | |
| | L-methionine | | | | | | | | | |
| | biosynthesis I | | | | | | | | | |
| GLUCUROCAT- | superpathway | 27 | 0.006872 | 5.77 | 57309.57 | 88753.82 | 1.00 | 9930.86 | 6803.57 | 1.00 |
| PWY | of &beta;-D- | | | | | | | | | |
| | glucuronide | | | | | | | | | |
| | and D- | | | | | | | | | |
| | glucuronate | | | | | | | | | |
| | degradation | | | | | | | | | |
| PWY0-781 | aspartate | 28 | 0.006856 | 5.28 | 50130.99 | 78655.15 | 1.00 | 9493.70 | 7322.25 | 1.00 |
| | superpathway | | | | | | | | | |
| ANAGLYCOLYSIS- | glycolysis III | 29 | 0.006699 | 3.97 | 157412.87 | 180880.09 | 1.00 | 39616.54 | 19547.45 | 1.00 |
| PWY | (from glucose) | | | | | | | | | |
| PWY-6123 | inosine-5′- | 30 | 0.006625 | 4.01 | 134834.30 | 154332.32 | 1.00 | 33605.59 | 16812.37 | 1.00 |
| | phosphate | | | | | | | | | |
| | biosynthesis I | | | | | | | | | |
| PWY-6121 | 5-aminoimidazole | 31 | 0.006358 | 3.97 | 143511.57 | 164255.51 | 1.00 | 36122.21 | 18777.40 | 1.00 |
| | ribonucleotide | | | | | | | | | |
| | biosynthesis I | | | | | | | | | |
| PWY-5384 | sucrose | 32 | 0.006001 | 4.90 | 59115.62 | 90240.55 | 1.00 | 12053.55 | 10048.97 | 1.00 |
| | degradation IV | | | | | | | | | |
| | (sucrose | | | | | | | | | |
| | phosphorylase) | | | | | | | | | |
| PWY-7222 | guanosine | 33 | 0.005852 | 4.40 | 171855.93 | 208910.59 | 1.00 | 39037.26 | 21505.96 | 1.00 |
| | deoxyribonucleotides | | | | | | | | | |
| | de novo | | | | | | | | | |
| | biosynthesis II | | | | | | | | | |
| PWY-7242 | D-fructuronate | 34 | 0.005446 | 5.36 | 108556.86 | 147295.93 | 1.00 | 20258.95 | 12667.63 | 1.00 |
| | degradation | | | | | | | | | |
| GOLPDLCAT- | superpathway | 35 | 0.005324 | 7.27 | 1469.95 | 8316.20 | 0.99 | 202.18 | 432.68 | 0.94 |
| PWY | of glycerol | | | | | | | | | |
| | degradation to | | | | | | | | | |
| | 1,3-propanediol | | | | | | | | | |
| PWY-7013 | L-1,2- | 36 | 0.005199 | 6.66 | 7931.24 | 30707.40 | 1.00 | 1190.11 | 2808.39 | 0.93 |
| | propanediol | | | | | | | | | |
| | degradation | | | | | | | | | |
| P124-PWY | Bifidobacterium | 37 | 0.005185 | 8.11 | 19432.49 | 42956.35 | 1.00 | 2397.16 | 3856.49 | 1.00 |
| | shunt | | | | | | | | | |
| PWY-6385 | peptidoglycan | 38 | 0.004984 | 3.91 | 127516.72 | 144162.90 | 1.00 | 32618.20 | 16544.58 | 1.00 |
| | biosynthesis III | | | | | | | | | |
| | (mycobacteria) | | | | | | | | | |
| PWY-5347 | superpathway of | 39 | 0.004923 | 4.75 | 71487.80 | 98197.58 | 1.00 | 15051.58 | 11206.66 | 1.00 |
| | L-methionine | | | | | | | | | |
| | biosynthesis | | | | | | | | | |
| | (transsulfuration) | | | | | | | | | |
| PWY-5104 | L-isoleucine | 40 | 0.004904 | 3.76 | 152215.73 | 17370.58 | 1.00 | 40466.33 | 21816.16 | 1.00 |
| | biosynthesis IV | | | | | | | | | |
| P122-PWY | heterolactic | 41 | 0.004838 | 8.05 | 15419.45 | 34825.46 | 1.00 | 1916.48 | 3035.80 | 1.00 |
| | fermentation | | | | | | | | | |
| PWY-3801 | sucrose | 42 | 0.004829 | 0.24 | 91.32 | 1509.62 | 0.44 | 383.94 | 1773.17 | 0.35 |
| | degradation II | | | | | | | | | |
| | (sucrose synthase) | | | | | | | | | |
| PEPTIDOGLYCANSYN- | peptidoglycan | 43 | 0.004794 | 3.91 | 128456.44 | 145009.61 | 1.00 | 32818.46 | 16611.34 | 1.00 |
| PWY | biosynthesis I | | | | | | | | | |
| | (meso- | | | | | | | | | |
| | diaminopimelate | | | | | | | | | |
| | containing) | | | | | | | | | |
| RIBOSYN2- | flavin | 44 | 0.004780 | 3.90 | 111745.27 | 131737.59 | 1.00 | 28641.46 | 15772.99 | 1.00 |
| PWY | biosynthesis I | | | | | | | | | |
| | (bacteria and | | | | | | | | | |
| | plants) | | | | | | | | | |
| TRNA-CHARGING- | tRNA charging | 45 | 0.004768 | 3.90 | 130149.53 | 147343.38 | 1.00 | 33409.36 | 17003.72 | 1.00 |
| PWY | | | | | | | | | | |
| PWY-7392 | taxadiene | 46 | 0.004744 | 4.98 | 23059.64 | 39103.88 | 1.00 | 4630.54 | 5243.38 | 1.00 |
| | biosynthesis | | | | | | | | | |
| | (engineered) | | | | | | | | | |
| REDCITCYC | TCA cycle VIII | 47 | 0.004735 | 9.15 | 18764.13 | 66020.56 | 1.00 | 2049.65 | 5087.08 | 1.00 |
| | (helicobacter) | | | | | | | | | |
| PWY-2942 | L-lysine | 48 | 0.004597 | 3.97 | 150432.02 | 171583.96 | 1.00 | 37924.77 | 18778.14 | 1.00 |
| | biosynthesis III | | | | | | | | | |
| PWY-6897 | thiamin | 49 | 0.004530 | 3.68 | 109681.12 | 128458.10 | 1.00 | 29830.89 | 20011.30 | 1.00 |
| | salvage II | | | | | | | | | |
| PWY-7663 | gondoate | 50 | 0.004456 | 4.16 | 179634.67 | 214427.08 | 1.00 | 43224.23 | 21636.87 | 1.00 |
| | biosynthesis | | | | | | | | | |
| | (anaerobic) | | | | | | | | | |
| TRPSYN- | L-tryptophan | 51 | 0.004398 | 3.88 | 115192.82 | 136948.27 | 1.00 | 29664.21 | 16817.20 | 1.00 |
| PWY | biosynthesis | | | | | | | | | |
| PWY-6387 | UDP-N- | 52 | 0.004364 | 3.92 | 129174.34 | 145705.81 | 1.00 | 32982.38 | 16688.93 | 1.00 |
| | acetylmuramoyl- | | | | | | | | | |
| | pentapeptide | | | | | | | | | |
| | biosynthesis I | | | | | | | | | |
| | (meso- | | | | | | | | | |
| | diaminopimelate | | | | | | | | | |
| | containing) | | | | | | | | | |
| PWY-621 | sucrose | 53 | 0.004344 | 4.44 | 128851.46 | 156513.66 | 1.00 | 29024.02 | 20621.37 | 1.00 |
| | degradation III | | | | | | | | | |
| | (sucrose invertase) | | | | | | | | | |
| DENOVOPURINE2- | superpathway of | 54 | 0.004273 | 5.42 | 106393.82 | 136843.29 | 1.00 | 19628.63 | 13926.57 | 1.00 |
| PWY | purine nucleotides | | | | | | | | | |
| | de novo | | | | | | | | | |
| | biosynthesis II | | | | | | | | | |
| PWY-7539 | 6-hydroxymethyl- | 55 | 0.004264 | 3.83 | 108766.15 | 131626.36 | 1.00 | 28384.93 | 19568.26 | 1.00 |
| | dihydropterin | | | | | | | | | |
| | diphosphate | | | | | | | | | |
| | biosynthesis III | | | | | | | | | |
| | (Chlamydia) | | | | | | | | | |
| PWY-6147 | 6-hydroxymethyl- | 56 | 0.004173 | 3.80 | 105610.99 | 126859.73 | 1.00 | 27827.44 | 19724.99 | 1.00 |
| | dihydropterin | | | | | | | | | |
| | diphosphate | | | | | | | | | |
| | biosynthesis I | | | | | | | | | |
| GLUCARGALACTSUPER- | superpathway | 57 | 0.004170 | 9.76 | 4466.91 | 21210.22 | 0.99 | 457.74 | 911.54 | 1.00 |
| PWY | of D-glucarate | | | | | | | | | |
| | and D-galactarate | | | | | | | | | |
| | degradation | | | | | | | | | |
| COA-PWY | coenzyme A | 58 | 0.004159 | 3.92 | 123757.36 | 139748.39 | 1.00 | 31573.49 | 15899.61 | 1.00 |
| | biosynthesis I | | | | | | | | | |
| GALACTARDEG- | D-galactarate | 59 | 0.004147 | 9.76 | 4466.91 | 21210.22 | 0.99 | 457.74 | 911.54 | 1.00 |
| PWY | degradation I | | | | | | | | | |
| PWY-7220 | adenosine | 60 | 0.004002 | 4.40 | 171855.93 | 208910.59 | 1.00 | 39037.26 | 21505.96 | 1.00 |
| | deoxyribonucleotides | | | | | | | | | |
| | de novo | | | | | | | | | |
| | biosynthesis II | | | | | | | | | |
| PWY-5973 | cis-vaccenate | 61 | 0.003973 | 4.04 | 161442.82 | 191518.09 | 1.00 | 39974.77 | 20024.39 | 1.00 |
| | biosynthesis | | | | | | | | | |
| PWY-6891 | thiazole | 62 | 0.003908 | 7.07 | 29308.66 | 58392.26 | 1.00 | 4147.43 | 4624.29 | 1.00 |
| | biosynthesis II | | | | | | | | | |
| | (Bacillus) | | | | | | | | | |
| GLUTORN- | L-ornithine | 63 | 0.003844 | 3.53 | 98956.14 | 117099.43 | 1.00 | 28029.94 | 21071.03 | 1.00 |
| PWY | biosynthesis | | | | | | | | | |
| PWY-7560 | methylerythritol | 64 | 0.003834 | 3.92 | 134182.83 | 154729.56 | 1.00 | 34272.25 | 17397.13 | 1.00 |
| | phosphate | | | | | | | | | |
| | pathway II | | | | | | | | | |
| PANTO- | phosphopantothenate | 65 | 0.003824 | 3.75 | 102917.05 | 125172.63 | 1.00 | 27427.71 | 17827.49 | 1.00 |
| PWY | biosynthesis I | | | | | | | | | |
| PWY0- | superpathway | 66 | 0.003812 | 4.19 | 70319.66 | 91976.78 | 1.00 | 16778.62 | 11799.80 | 1.00 |
| 1297 | of purine | | | | | | | | | |
| | deoxyribonucleosides | | | | | | | | | |
| | degradation | | | | | | | | | |
| PWY-7208 | superpathway | 67 | 0.003802 | 3.81 | 146949.20 | 165685.83 | 1.00 | 38548.99 | 20856.81 | 1.00 |
| | of pyrimidine | | | | | | | | | |
| | nucleobases | | | | | | | | | |
| | salvage | | | | | | | | | |
| PWY-181 | photorespiration | 68 | 0.003727 | 1.86 | 260.96 | 3493.16 | 0.55 | 140.42 | 530.40 | 0.58 |
| NONMEVIPP- | methylerythritol- | 69 | 0.003667 | 3.92 | 134182.83 | 154729.56 | 1.00 | 34272.25 | 17397.13 | 1.00 |
| PWY | phosphate | | | | | | | | | |
| | pathway I | | | | | | | | | |
| FUC-RHAMCAT- | superpathway | 70 | 0.003625 | 5.03 | 30651.10 | 41677.69 | 1.00 | 6087.89 | 4750.90 | 1.00 |
| PWY | of fucose and | | | | | | | | | |
| | rhamnose | | | | | | | | | |
| | degradation | | | | | | | | | |
| PWY-6895 | superpathway | 71 | 0.003621 | 6.13 | 51188.48 | 76665.18 | 1.00 | 8355.66 | 7332.84 | 1.00 |
| | of thiamin | | | | | | | | | |
| | diphosphate | | | | | | | | | |
| | biosynthesis II | | | | | | | | | |
| ARGORNPROST- | arginine, | 72 | 0.003543 | 4.02 | 5675.54 | 8978.45 | 1.00 | 1410.86 | 2008.22 | 1.00 |
| PWY | ornithine and | | | | | | | | | |
| | proline | | | | | | | | | |
| | interconversion | | | | | | | | | |
| PWY-6703 | preQ0 | 73 | 0.003505 | 3.89 | 82160.52 | 110131.25 | 1.00 | 21142.14 | 17391.99 | 1.00 |
| | biosynthesis | | | | | | | | | |
| PWY-7219 | adenosine | 74 | 0.003491 | 3.92 | 150642.61 | 171191.32 | 1.00 | 38393.50 | 19673.06 | 1.00 |
| | ribonucleotides | | | | | | | | | |
| | de novo | | | | | | | | | |
| | biosynthesis | | | | | | | | | |
| FOLSYN- | superpathway of | 75 | 0.003452 | 4.00 | 115361.09 | 138014.61 | 1.00 | 28875.19 | 16869.28 | 1.00 |
| PWY | tetrahydrofolate | | | | | | | | | |
| | biosynthesis | | | | | | | | | |
| | and salvage | | | | | | | | | |
| PWY0-41 | allantoin | 76 | 0.003451 | 15.37 | 3484.63 | 18488.68 | 0.99 | 226.74 | 473.49 | 0.93 |
| | degradation IV | | | | | | | | | |
| | (anaerobic) | | | | | | | | | |
| PYRIDNUCSYN- | NAD | 77 | 0.003421 | 3.88 | 111276.25 | 130486.87 | 1.00 | 28698.20 | 15283.44 | 1.00 |
| PWY | biosynthesis I | | | | | | | | | |
| | (from aspartate) | | | | | | | | | |
| PWY-7229 | superpathway | 78 | 0.003373 | 4.02 | 157364.23 | 181723.85 | 1.00 | 39152.22 | 20656.79 | 1.00 |
| | of adenosine | | | | | | | | | |
| | nucleotides de | | | | | | | | | |
| | novo | | | | | | | | | |
| | biosynthesis I | | | | | | | | | |
| PWY-6737 | starch | 79 | 0.003316 | 4.04 | 187661.83 | 223111.56 | 1.00 | 46433.64 | 24911.85 | 1.00 |
| | degradation V | | | | | | | | | |
| P221-PWY | octane oxidation | 80 | 0.003294 | 3.00 | 5761.91 | 12779.90 | 0.97 | 1918.80 | 2782.48 | 0.94 |
| PWY-5188 | tetrapyrrole | 81 | 0.003292 | 2.76 | 48681.91 | 67878.32 | 1.00 | 17660.45 | 15639.69 | 1.00 |
| | biosynthesis I | | | | | | | | | |
| | (from glutamate) | | | | | | | | | |
| PWY-7199 | pyrimidine | 82 | 0.003273 | 5.15 | 91237.42 | 116483.90 | 1.00 | 17703.08 | 11591.36 | 1.00 |
| | deoxyribonucleosides | | | | | | | | | |
| | salvage | | | | | | | | | |
| GLYCOGENSYNTH- | glycogen | 83 | 0.003266 | 3.47 | 131973.83 | 153409.27 | 1.00 | 38040.27 | 19979.74 | 1.00 |
| PWY | biosynthesis I | | | | | | | | | |
| | (from ADP-D- | | | | | | | | | |
| | Glucose) | | | | | | | | | |
| VALDEG- | L-valine | 84 | 0.003235 | 11.83 | 36.29 | 189.04 | 0.39 | 3.07 | 23.73 | 0.18 |
| PWY | degradation I | | | | | | | | | |
| P164-PWY | purine | 85 | 0.003099 | 3.91 | 31488.58 | 50403.11 | 1.00 | 8061.70 | 6927.39 | 1.00 |
| | nucleobases | | | | | | | | | |
| | degradation I | | | | | | | | | |
| | (anaerobic) | | | | | | | | | |
| PWY-6471 | peptidoglycan | 86 | 0.003092 | 4.06 | 47677.60 | 63879.56 | 1.00 | 11749.05 | 9689.53 | 1.00 |
| | biosynthesis IV | | | | | | | | | |
| | (Enterococcus | | | | | | | | | |
| | faecium) | | | | | | | | | |
| P441-PWY | superpathway of | 87 | 0.003085 | 4.80 | 48761.56 | 68746.80 | 1.00 | 10157.31 | 8805.55 | 1.00 |
| | N-acetylneuraminate | | | | | | | | | |
| | degradation | | | | | | | | | |
| PWY-6163 | chorismate | 88 | 0.003063 | 3.75 | 136555.50 | 153849.19 | 1.00 | 36411.39 | 19257.17 | 1.00 |
| | biosynthesis | | | | | | | | | |
| | from 3- | | | | | | | | | |
| | dehydroquinate | | | | | | | | | |
| PWY-7187 | pyrimidine | 89 | 0.003060 | 5.06 | 69465.91 | 89894.29 | 1.00 | 13715.69 | 9154.58 | 1.00 |
| | deoxyribonucleotides | | | | | | | | | |
| | de novo | | | | | | | | | |
| | biosynthesis II | | | | | | | | | |
| PWY0- | methylphosphonate | 90 | 0.003047 | 21.15 | 9321.47 | 52007.08 | 0.99 | 440.83 | 961.71 | 1.00 |
| 1533 | degradation I | | | | | | | | | |
| PWY-7332 | superpathway | 91 | 0.003044 | 2.20 | 4098.00 | 13921.69 | 0.98 | 1866.86 | 3130.64 | 0.99 |
| | of UDP-N- | | | | | | | | | |
| | acetylglucosamine- | | | | | | | | | |
| | derived O-antigen | | | | | | | | | |
| | building blocks | | | | | | | | | |
| | biosynthesis | | | | | | | | | |
| CALVIN- | Calvin- | 92 | 0.003036 | 3.73 | 156447.97 | 176810.12 | 1.00 | 41919.47 | 23350.92 | 1.00 |
| PWY | Benson- | | | | | | | | | |
| | Bassham cycle | | | | | | | | | |
| PWY-3001 | superpathway | 93 | 0.003021 | 3.93 | 144728.31 | 165925.82 | 1.00 | 36808.04 | 19082.06 | 1.00 |
| | of L-isoleucine | | | | | | | | | |
| | biosynthesis I | | | | | | | | | |
| GLCMANNANAUT- | superpathway of | 94 | 0.003016 | 3.98 | 55949.28 | 66026.69 | 1.00 | 14050.92 | 9665.99 | 1.00 |
| PWY | N-acetylglucosamine, | | | | | | | | | |
| | N-acetylmannosamine | | | | | | | | | |
| | and N-acetylneuraminate | | | | | | | | | |
| | degradation | | | | | | | | | |
| PWY-6263 | superpathway | 95 | 0.002974 | 3.51 | 7706.21 | 15896.50 | 0.95 | 2196.54 | 3113.85 | 0.95 |
| | of menaquinol- | | | | | | | | | |
| | 8 biosynthesis II | | | | | | | | | |
| P562-PWY | myo-inositol | 96 | 0.002962 | 1.27 | 9881.76 | 42205.37 | 1.00 | 7805.01 | 18297.70 | 1.00 |
| | degradation I | | | | | | | | | |
| PWY-5686 | UMP | 97 | 0.002937 | 3.82 | 146755.20 | 166508.73 | 1.00 | 38378.86 | 20600.14 | 1.00 |
| | biosynthesis | | | | | | | | | |
| PWY-6126 | superpathway | 98 | 0.002929 | 4.02 | 150054.04 | 173856.36 | 1.00 | 37349.40 | 19833.46 | 1.00 |
| | of adenosine | | | | | | | | | |
| | nucleotides de | | | | | | | | | |
| | novo | | | | | | | | | |
| | biosynthesis II | | | | | | | | | |
| PWY-5531 | chlorophyllide | 99 | 0.002923 | 0.40 | 16.30 | 94.77 | 0.42 | 40.43 | 387.08 | 0.45 |
| | a biosynthesis | | | | | | | | | |
| | II (anaerobic) | | | | | | | | | |
| GLUCARDEG- | D-glucarate | 100 | 0.002911 | 6.88 | 4907.86 | 21407.69 | 1.00 | 713.12 | 954.82 | 1.00 |
| PWY | degradation I | | | | | | | | | |

IBD = Inflammatory Bowel Disease;
IBS = Irritable Bowel Syndrome;
RFFR = Random Forest Feature Rank;
# = Rank;
MDA = Mean Decrease Accuracy;
FC = Fold Change;
M = Mean;
SD = Standard Deviation;
DP = Detection Prevalence;

TABLE 3
IBD vs CDI delineation features - (metabolic pathway)

| | | | | FC | | | | | | |
|---|---|---|---|---|---|---|---|---|---|---|
| MetaCyc | Pathway | RFFR | | IBD | Abundance (IBD) | | | Abundance (CDI) | | |
| Pathway ID | description | # | MDA | vs CDI | M | SD | DP | M | SD | DP |
| PWY-6151 | S-adenosyl-L- | 1 | 0.022939 | 11.44 | 107683.22 | 124365.32 | 1.00 | 9411.61 | 7522.84 | 1.00 |
| | methionine | | | | | | | | | |
| | cycle I | | | | | | | | | |
| P221-PWY | octane oxidation | 2 | 0.021292 | 16.52 | 5761.91 | 12779.90 | 0.97 | 348.70 | 1946.70 | 0.61 |
| PWY-5505 | L-glutamate and | 3 | 0.020324 | 13.15 | 96135.88 | 117872.52 | 1.00 | 7310.25 | 7222.51 | 1.00 |
| | L-glutamine | | | | | | | | | |
| | biosynthesis | | | | | | | | | |
| PWY-5121 | superpathway of | 4 | 0.019277 | 11.33 | 127725.86 | 145558.38 | 1.00 | 11275.00 | 9094.58 | 1.00 |
| | geranylgeranyl | | | | | | | | | |
| | diphosphate | | | | | | | | | |
| | biosynthesis II | | | | | | | | | |
| | (via MEP) | | | | | | | | | |
| GLYCOGENSYNTH- | glycogen | 5 | 0.017609 | 11.84 | 131973.83 | 153409.27 | 1.00 | 11149.92 | 9865.46 | 1.00 |
| PWY | biosynthesis I | | | | | | | | | |
| | (from ADP-D- | | | | | | | | | |
| | Glucose) | | | | | | | | | |
| NONMEVIPP- | methylerythritol | 6 | 0.017089 | 11.41 | 134182.83 | 154729.56 | 1.00 | 11765.12 | 9593.11 | 1.00 |
| PWY | phosphate | | | | | | | | | |
| | pathway I | | | | | | | | | |
| PWY-6269 | adenosylcobalamin | 7 | 0.016927 | 11.30 | 98727.92 | 112284.20 | 1.00 | 8739.20 | 7664.91 | 1.00 |
| | salvage from | | | | | | | | | |
| | cobinamide II | | | | | | | | | |
| PYRIDNUCSYN- | NAD | 8 | 0.014565 | 12.04 | 111276.25 | 130486.87 | 1.00 | 9244.66 | 7697.40 | 1.00 |
| PWY | biosynthesis I | | | | | | | | | |
| | (from aspartate) | | | | | | | | | |
| COA-PWY | coenzyme A | 9 | 0.014128 | 10.61 | 123757.36 | 139748.39 | 1.00 | 11669.39 | 8761.12 | 1.00 |
| | biosynthesis I | | | | | | | | | |
| PWY-5686 | UMP | 10 | 0.012670 | 10.63 | 146755.20 | 166508.73 | 1.00 | 13799.67 | 10365.94 | 1.00 |
| | biosynthesis | | | | | | | | | |
| PWY-7560 | methylerythritol | 11 | 0.012584 | 11.41 | 134182.83 | 154729.56 | 1.00 | 11765.12 | 9593.11 | 1.00 |
| | phosphate | | | | | | | | | |
| | pathway II | | | | | | | | | |
| PWY-6386 | UDP-N- | 12 | 0.012251 | 10.49 | 129151.67 | 145723.67 | 1.00 | 12310.17 | 9381.48 | 1.00 |
| | acetylmuramoyl- | | | | | | | | | |
| | pentapeptide | | | | | | | | | |
| | biosynthesis II | | | | | | | | | |
| | (lysine-containing) | | | | | | | | | |
| PWY-6277 | superpathway of | 13 | 0.011979 | 10.45 | 138322.07 | 155913.44 | 1.00 | 13242.31 | 10142.08 | 1.00 |
| | 5-aminoimidazole | | | | | | | | | |
| | ribonucleotide | | | | | | | | | |
| | biosynthesis | | | | | | | | | |
| PWY-5509 | adenosylcobalamin | 14 | 0.011397 | 11.34 | 98134.29 | 111713.94 | 1.00 | 8656.86 | 7613.93 | 1.00 |
| | biosynthesis from | | | | | | | | | |
| | cobyrinate a,c- | | | | | | | | | |
| | diamide I | | | | | | | | | |
| PWY-7219 | adenosine | 15 | 0.010926 | 10.56 | 150642.61 | 171191.32 | 1.00 | 14262.17 | 10745.91 | 1.00 |
| | ribonucleotides | | | | | | | | | |
| | de novo | | | | | | | | | |
| | biosynthesis | | | | | | | | | |
| COBALSYN- | adenosylcobalamin | 16 | 0.010834 | 10.70 | 101783.99 | 114902.71 | 1.00 | 9508.59 | 8221.80 | 1.00 |
| PWY | salvage from | | | | | | | | | |
| | cobinamide I | | | | | | | | | |
| PWY-6163 | chorismate | 17 | 0.010562 | 10.79 | 136555.50 | 153849.19 | 1.00 | 12651.08 | 9738.48 | 1.00 |
| | biosynthesis | | | | | | | | | |
| | from 3- | | | | | | | | | |
| | dehydroquinate | | | | | | | | | |
| PWY-5861 | superpathway of | 18 | 0.010286 | 4.23 | 16460.72 | 52245.15 | 1.00 | 3894.44 | 4510.05 | 0.99 |
| | demethylmenaquinol- | | | | | | | | | |
| | 8 biosynthesis | | | | | | | | | |
| PWY-6123 | inosine-5′- | 19 | 0.010226 | 10.67 | 134834.30 | 154332.32 | 1.00 | 12632.90 | 9679.61 | 1.00 |
| | phosphate | | | | | | | | | |
| | biosynthesis I | | | | | | | | | |
| PWY-5837 | 1,4-dihydroxy- | 20 | 0.010086 | 4.03 | 12463.62 | 49647.52 | 1.00 | 3094.02 | 3998.36 | 1.00 |
| | 2-naphthoate | | | | | | | | | |
| | biosynthesis I | | | | | | | | | |
| PWY-5863 | superpathway | 21 | 0.010056 | 4.04 | 13008.58 | 49499.71 | 1.00 | 3219.88 | 3983.44 | 1.00 |
| | of phylloquinol | | | | | | | | | |
| | biosynthesis | | | | | | | | | |
| GLYCOCAT- | glycogen | 22 | 0.009741 | 11.42 | 159288.63 | 184015.95 | 1.00 | 13942.78 | 11948.63 | 1.00 |
| PWY | degradation I | | | | | | | | | |
| | (bacterial) | | | | | | | | | |
| ARO-PWY | chorismate | 23 | 0.009344 | 10.36 | 130093.56 | 146728.47 | 1.00 | 12557.51 | 9830.31 | 1.00 |
| | biosynthesis I | | | | | | | | | |
| PWY-5897 | superpathway | 24 | 0.009053 | 4.31 | 17927.39 | 53168.30 | 1.00 | 4162.12 | 4654.22 | 1.00 |
| | of menaquinol- | | | | | | | | | |
| | 11 biosynthesis | | | | | | | | | |
| PWY-6317 | galactose | 25 | 0.008715 | 10.53 | 117717.50 | 139097.49 | 1.00 | 11183.74 | 9380.07 | 1.00 |
| | degradation I | | | | | | | | | |
| | (Leloir pathway) | | | | | | | | | |
| PWY-5840 | superpathway | 26 | 0.008484 | 4.41 | 18365.80 | 52805.78 | 1.00 | 4165.06 | 4482.90 | 1.00 |
| | of menaquinol- | | | | | | | | | |
| | 7 biosynthesis | | | | | | | | | |
| PWY-5097 | L-lysine | 27 | 0.008451 | 10.58 | 147736.56 | 168639.95 | 1.00 | 13962.57 | 10737.14 | 1.00 |
| | biosynthesis VI | | | | | | | | | |
| PWY-6385 | peptidoglycan | 28 | 0.008358 | 10.42 | 127516.72 | 144162.90 | 1.00 | 12234.28 | 9272.55 | 1.00 |
| | biosynthesis III | | | | | | | | | |
| | (mycobacteria) | | | | | | | | | |
| COMPLETE-ARO- | superpathway | 29 | 0.008283 | 10.41 | 136769.77 | 154592.26 | 1.00 | 13134.09 | 10317.24 | 1.00 |
| PWY | of aromatic | | | | | | | | | |
| | amino acid | | | | | | | | | |
| | biosynthesis | | | | | | | | | |
| PWY-6892 | thiazole | 30 | 0.008222 | 12.69 | 119155.10 | 141683.42 | 1.00 | 9387.20 | 7923.11 | 1.00 |
| | biosynthesis I | | | | | | | | | |
| | (E. coli) | | | | | | | | | |
| PEPTIDOGLYCANSYN- | peptidoglycan | 31 | 0.007798 | 10.38 | 128456.44 | 145009.61 | 1.00 | 12377.64 | 9359.46 | 1.00 |
| PWY | biosynthesis I | | | | | | | | | |
| | (meso- | | | | | | | | | |
| | diaminopimelate | | | | | | | | | |
| | containing) | | | | | | | | | |
| PWY-6122 | 5-aminoimidazole | 32 | 0.007775 | 10.45 | 138322.07 | 155913.44 | 1.00 | 13242.31 | 10142.08 | 1.00 |
| | ribonucleotide | | | | | | | | | |
| | biosynthesis II | | | | | | | | | |
| PWY-5899 | superpathway | 33 | 0.007749 | 4.31 | 17927.39 | 53168.30 | 1.00 | 4162.12 | 4654.22 | 1.00 |
| | of menaquinol- | | | | | | | | | |
| | 13 biosynthesis | | | | | | | | | |
| PYRIDNUCSAL- | NAD salvage | 34 | 0.007732 | 12.01 | 114532.43 | 133167.55 | 1.00 | 9539.35 | 8058.22 | 1.00 |
| PWY | pathway I | | | | | | | | | |
| PWY-5838 | superpathway | 35 | 0.007583 | 4.37 | 18629.77 | 53691.09 | 1.00 | 4262.18 | 4746.55 | 0.99 |
| | of menaquinol- | | | | | | | | | |
| | 8 biosynthesis I | | | | | | | | | |
| PWY-5667 | CDP- | 36 | 0.007056 | 10.07 | 145849.97 | 165605.02 | 1.00 | 14479.76 | 11114.39 | 1.00 |
| | diacylglycerol | | | | | | | | | |
| | biosynthesis I | | | | | | | | | |
| PWY0- | methylphosphonate | 37 | 0.007041 | 3.60 | 9321.47 | 52007.08 | 0.99 | 2590.86 | 3843.40 | 1.00 |
| 1533 | degradation I | | | | | | | | | |
| PWY-6387 | UDP-N- | 38 | 0.006878 | 10.33 | 129174.34 | 145705.81 | 1.00 | 12506.69 | 9467.25 | 1.00 |
| | acetylmuramoyl- | | | | | | | | | |
| | pentapeptide | | | | | | | | | |
| | biosynthesis I | | | | | | | | | |
| | (meso- | | | | | | | | | |
| | diaminopimelate | | | | | | | | | |
| | containing) | | | | | | | | | |
| TRNA-CHARGING- | tRNA charging | 39 | 0.006824 | 10.43 | 130149.53 | 147343.38 | 1.00 | 12475.49 | 9660.68 | 1.00 |
| PWY | | | | | | | | | | |
| PWY-5100 | pyruvate | 40 | 0.006692 | 10.51 | 135870.24 | 152815.93 | 1.00 | 12921.80 | 9801.95 | 1.00 |
| | fermentation to | | | | | | | | | |
| | acetate and | | | | | | | | | |
| | lactate II | | | | | | | | | |
| PWY-7663 | gondoate | 41 | 0.006475 | 10.95 | 179634.67 | 214427.08 | 1.00 | 16407.13 | 12555.92 | 1.00 |
| | biosynthesis | | | | | | | | | |
| | (anaerobic) | | | | | | | | | |
| PWY-5104 | L-isoleucine | 42 | 0.006468 | 11.05 | 152215.73 | 173701.58 | 1.00 | 13779.72 | 11296.81 | 1.00 |
| | biosynthesis IV | | | | | | | | | |
| PWY-5973 | cis-vaccenate | 43 | 0.006339 | 10.67 | 161442.82 | 191518.09 | 1.00 | 15125.38 | 11673.99 | 1.00 |
| | biosynthesis | | | | | | | | | |
| PWY-5898 | superpathway | 44 | 0.006194 | 4.31 | 17927.39 | 53168.30 | 1.00 | 4162.12 | 4654.22 | 1.00 |
| | of menaquinol- | | | | | | | | | |
| | 12 biosynthesis | | | | | | | | | |
| PWY0- | CDP- | 45 | 0.006165 | 10.07 | 145849.97 | 165605.02 | 1.00 | 14479.76 | 11114.39 | 1.00 |
| 1319 | diacylglycerol | | | | | | | | | |
| | biosynthesis II | | | | | | | | | |
| PWY-7221 | guanosine | 46 | 0.006083 | 10.56 | 139893.46 | 161207.20 | 1.00 | 13250.25 | 10071.10 | 1.00 |
| | ribonucleotides | | | | | | | | | |
| | de novo | | | | | | | | | |
| | biosynthesis | | | | | | | | | |
| PWY4FS-8 | phosphatidylglycerol | 47 | 0.005787 | 9.66 | 118520.55 | 138335.79 | 1.00 | 12271.48 | 9713.41 | 1.00 |
| | biosynthesis II | | | | | | | | | |
| | (non-plastidic) | | | | | | | | | |
| PWY-6737 | starch | 48 | 0.005476 | 12.22 | 187661.83 | 223111.56 | 1.00 | 15361.05 | 13716.01 | 1.00 |
| | degradation V | | | | | | | | | |
| PWY-3001 | superpathway | 49 | 0.005435 | 10.91 | 144728.31 | 165925.82 | 1.00 | 13263.33 | 10722.63 | 1.00 |
| | of L-isoleucine | | | | | | | | | |
| | biosynthesis I | | | | | | | | | |
| HISTSYN- | L-histidine | 50 | 0.005147 | 11.62 | 125494.23 | 146173.13 | 1.00 | 10804.07 | 8915.40 | 1.00 |
| PWY | biosynthesis | | | | | | | | | |
| PWY-7208 | superpathway | 51 | 0.005014 | 10.30 | 146949.20 | 165685.83 | 1.00 | 14263.75 | 10855.98 | 1.00 |
| | of pyrimidine | | | | | | | | | |
| | nucleobases | | | | | | | | | |
| | salvage | | | | | | | | | |
| PHOSLIPSYN- | superpathway of | 52 | 0.004984 | 9.85 | 132254.48 | 15309.74 | 1.00 | 13420.46 | 10360.64 | 1.00 |
| PWY | phospholipid | | | | | | | | | |
| | biosynthesis I | | | | | | | | | |
| | (bacteria) | | | | | | | | | |
| PWY-6121 | 5-aminoimidazole | 53 | 0.004984 | 10.53 | 143511.57 | 164255.51 | 1.00 | 13634.17 | 10455.30 | 1.00 |
| | ribonucleotide | | | | | | | | | |
| | biosynthesis I | | | | | | | | | |
| PWY-2942 | L-lysine | 54 | 0.004862 | 10.45 | 150432.02 | 171583.96 | 1.00 | 14401.68 | 11021.52 | 1.00 |
| | biosynthesis III | | | | | | | | | |
| PWY-5850 | superpathway | 55 | 0.004651 | 4.37 | 13692.01 | 50596.90 | 0.99 | 3131.89 | 4208.09 | 0.98 |
| | of menaquinol- | | | | | | | | | |
| | 6 biosynthesis I | | | | | | | | | |
| SER-GLYSYN- | superpathway | 56 | 0.004639 | 10.88 | 127879.50 | 145388.75 | 1.00 | 11750.83 | 10070.81 | 1.00 |
| PWY | of L-serine and | | | | | | | | | |
| | glycine | | | | | | | | | |
| | biosynthesis I | | | | | | | | | |
| AEROBACTINSYN- | aerobactin | 57 | 0.004630 | 10.50 | 1057.02 | 12646.97 | 0.66 | 100.68 | 344.92 | 0.76 |
| PWY | biosynthesis | | | | | | | | | |
| PWY-5860 | superpathway of | 58 | 0.004627 | 4.37 | 12152.27 | 49430.15 | 0.99 | 2779.78 | 3938.60 | 0.98 |
| | demethylmenaquinol- | | | | | | | | | |
| | 6 biosynthesis I | | | | | | | | | |
| PWY-6126 | superpathway | 59 | 0.004418 | 10.19 | 150054.04 | 173856.36 | 1.00 | 14721.48 | 11085.79 | 1.00 |
| | of adenosine | | | | | | | | | |
| | nucleotides de | | | | | | | | | |
| | novo | | | | | | | | | |
| | biosynthesis II | | | | | | | | | |
| DAPLYSINESYN- | L-lysine | 60 | 0.004098 | 9.47 | 96805.58 | 114220.94 | 1.00 | 10219.74 | 8756.71 | 1.00 |
| PWY | biosynthesis I | | | | | | | | | |
| NONOXIPENT- | pentose phosphate | 61 | 0.004084 | 9.82 | 193969.86 | 231579.44 | 1.00 | 19746.16 | 15361.51 | 1.00 |
| PWY | pathway (non- | | | | | | | | | |
| | oxidative branch) | | | | | | | | | |
| PWY-5862 | superpathway of | 62 | 0.004033 | 4.37 | 12153.96 | 49429.80 | 0.99 | 2779.78 | 3938.60 | 0.98 |
| | demethylmenaquinol- | | | | | | | | | |
| | 9 biosynthesis | | | | | | | | | |
| BRANCHED- | superpathway | 63 | 0.003957 | 10.75 | 144717.57 | 166226.20 | 1.00 | 13457.08 | 11094.56 | 1.00 |
| CHAIN- | of branched | | | | | | | | | |
| AA-SYN-PWY | amino acid | | | | | | | | | |
| | biosynthesis | | | | | | | | | |
| PWY-5845 | superpathway | 64 | 0.003895 | 4.37 | 13694.42 | 50596.39 | 0.99 | 3131.89 | 4208.09 | 0.98 |
| | of menaquinol- | | | | | | | | | |
| | 9 biosynthesis | | | | | | | | | |
| PWY-7229 | superpathway | 65 | 0.003788 | 10.20 | 157364.23 | 181723.85 | 1.00 | 15431.50 | 11650.85 | 1.00 |
| | of adenosine | | | | | | | | | |
| | nucleotides de | | | | | | | | | |
| | novo | | | | | | | | | |
| | biosynthesis I | | | | | | | | | |
| ARGSYNBSUB- | L-arginine | 66 | 0.003781 | 10.18 | 87902.52 | 107302.81 | 1.00 | 8634.15 | 7616.24 | 1.00 |
| PWY | biosynthesis II | | | | | | | | | |
| | (acetyl cycle) | | | | | | | | | |
| VALSYN- | L-valine | 67 | 0.003573 | 10.56 | 152045.37 | 173516.28 | 1.00 | 14398.84 | 11832.67 | 1.00 |
| PWY | biosynthesis | | | | | | | | | |
| PROTOCATECHUATE- | protocatechuate | 68 | 0.003571 | 2.74 | 1541.18 | 13248.14 | 0.78 | 563.24 | 1894.30 | 0.81 |
| ORTHO- | degradation II | | | | | | | | | |
| CLEAVAGE-PWY | (ortho-cleavage | | | | | | | | | |
| | pathway) | | | | | | | | | |
| ILEUSYN- | L-isoleucine | 69 | 0.003556 | 10.56 | 152045.37 | 173516.28 | 1.00 | 14398.84 | 11832.67 | 1.00 |
| PWY | biosynthesis I | | | | | | | | | |
| | (from threonine) | | | | | | | | | |
| 1CMET2- | N10-formyl- | 70 | 0.003516 | 10.82 | 125376.15 | 144992.96 | 1.00 | 11590.50 | 9125.00 | 1.00 |
| PWY | tetrahydrofolate | | | | | | | | | |
| | biosynthesis | | | | | | | | | |
| GALLATE- | gallate | 71 | 0.003490 | 6.55 | 327.66 | 5244.51 | 0.40 | 50.04 | 171.58 | 0.62 |
| DEGRADATION- | degradation I | | | | | | | | | |
| II-PWY | | | | | | | | | | |
| 3- | 4- | 72 | 0.003424 | 14.69 | 7016.55 | 47433.58 | 0.83 | 477.80 | 1818.62 | 0.89 |
| HYDROXYPHENYLACETATE- | hydroxyphenylacetate | | | | | | | | | |
| DEGRADATION-PWY | degradation | | | | | | | | | |
| METHYLGALLATE- | methylgallate | 73 | 0.003355 | 6.70 | 416.35 | 6281.48 | 0.53 | 62.13 | 211.04 | 0.64 |
| DEGRADATION-PWY | degradation | | | | | | | | | |
| PWY-5896 | superpathway | 74 | 0.003350 | 4.37 | 13692.01 | 50596.90 | 0.99 | 3131.89 | 4208.09 | 0.98 |
| | of menaquinol- | | | | | | | | | |
| | 10 biosynthesis | | | | | | | | | |
| PPGPPMET- | ppGpp | 75 | 0.003332 | 4.26 | 13669.70 | 58706.41 | 0.96 | 3209.93 | 4853.66 | 0.96 |
| PWY | biosynthesis | | | | | | | | | |
| ALL- | superpathway | 76 | 0.003329 | 4.37 | 13199.81 | 55694.03 | 0.93 | 3017.70 | 4247.08 | 0.95 |
| CHORISMATE- | of chorismate | | | | | | | | | |
| PWY | metabolism | | | | | | | | | |
| PWY-7400 | L-arginine | 77 | 0.003315 | 10.39 | 94035.64 | 110597.42 | 1.00 | 9053.26 | 7680.28 | 1.00 |
| | biosynthesis IV | | | | | | | | | |
| | (archaebacteria) | | | | | | | | | |
| THRESYN- | superpathway | 78 | 0.003298 | 10.96 | 142354.23 | 163498.18 | 1.00 | 12990.68 | 10283.94 | 1.00 |
| PWY | of L-threonine | | | | | | | | | |
| | biosynthesis | | | | | | | | | |
| DENOVOPURINE2- | superpathway of | 79 | 0.003294 | 9.33 | 106393.82 | 136843.29 | 1.00 | 11398.08 | 9115.62 | 1.00 |
| PWY | purine nucleotides | | | | | | | | | |
| | de novo | | | | | | | | | |
| | biosynthesis II | | | | | | | | | |
| ARGSYN- | L-arginine | 80 | 0.003257 | 10.36 | 93779.91 | 110424.01 | 1.00 | 9052.44 | 7686.03 | 1.00 |
| PWY | biosynthesis I | | | | | | | | | |
| | (via L-ornithine) | | | | | | | | | |
| PWY-7210 | pyrimidine | 81 | 0.003243 | 9.87 | 17450.16 | 22166.30 | 0.99 | 1768.37 | 2590.34 | 0.92 |
| | deoxyribonucleotides | | | | | | | | | |
| | biosynthesis | | | | | | | | | |
| | from CTP | | | | | | | | | |
| GLUCARDEG- | D-glucarate | 82 | 0.003177 | 2.22 | 4907.86 | 21407.69 | 1.00 | 2209.18 | 3044.60 | 0.99 |
| PWY | degradation I | | | | | | | | | |
| PWY-7198 | pyrimidine | 83 | 0.003093 | 9.75 | 14466.52 | 18873.23 | 0.99 | 1483.19 | 2294.17 | 0.92 |
| | deoxyribonucleotides | | | | | | | | | |
| | de novo | | | | | | | | | |
| | biosynthesis IV | | | | | | | | | |
| POLYAMINSYN3- | superpathway | 84 | 0.003047 | 11.06 | 12425.67 | 20513.46 | 1.00 | 1123.63 | 1443.18 | 1.00 |
| PWY | of polyamine | | | | | | | | | |
| | biosynthesis II | | | | | | | | | |
| PWY4FS-7 | phosphatidylglycerol | 85 | 0.003018 | 9.66 | 118520.55 | 138335.79 | 1.00 | 12271.48 | 9713.41 | 1.00 |
| | biosynthesis I | | | | | | | | | |
| | (plastidic) | | | | | | | | | |
| PWY0-166 | superpathway of | 86 | 0.002971 | 8.20 | 67662.59 | 88147.86 | 1.00 | 8253.14 | 6687.14 | 1.00 |
| | pyrimidine | | | | | | | | | |
| | deoxyribonucleotides | | | | | | | | | |
| | de novo | | | | | | | | | |
| | biosynthesis | | | | | | | | | |
| | (E. coli) | | | | | | | | | |
| GALLATE- | gallate | 87 | 0.002955 | 6.84 | 343.14 | 5245.90 | 0.53 | 50.17 | 171.78 | 0.64 |
| DEGRADATION-I- | degradation II | | | | | | | | | |
| PWY | | | | | | | | | | |
| PWY-5181 | toluene | 88 | 0.002921 | 4.52 | 1307.02 | 11902.37 | 0.72 | 289.01 | 672.79 | 0.78 |
| | degradation III | | | | | | | | | |
| | (aerobic) (via | | | | | | | | | |
| | p-cresol) | | | | | | | | | |
| GLUCOSE1PMETAB- | glucose and | 89 | 0.002879 | 4.60 | 15548.88 | 53407.74 | 0.99 | 3377.19 | 4632.05 | 0.98 |
| PWY | glucose-1- | | | | | | | | | |
| | phosphate | | | | | | | | | |
| | degradation | | | | | | | | | |
| CALVIN- | Calvin- | 90 | 0.002853 | 9.87 | 156447.97 | 176810.12 | 1.00 | 15850.71 | 12125.61 | 1.00 |
| PWY | Benson- | | | | | | | | | |
| | Bassham cycle | | | | | | | | | |
| PANTOSYN- | pantothenate | 91 | 0.002826 | 11.18 | 107386.72 | 127772.17 | 1.00 | 9601.39 | 7781.20 | 1.00 |
| PWY | and coenzyme | | | | | | | | | |
| | A biosynthesis I | | | | | | | | | |
| PWY-5484 | glycolysis II | 92 | 0.002816 | 9.81 | 126725.58 | 161436.81 | 1.00 | 12922.99 | 10052.31 | 1.00 |
| | (from fructose | | | | | | | | | |
| | 6-phosphate) | | | | | | | | | |
| PWY-5417 | catechol | 93 | 0.002813 | 1.73 | 540.85 | 6287.09 | 0.71 | 313.16 | 1142.85 | 0.79 |
| | degradation III | | | | | | | | | |
| | (ortho-cleavage | | | | | | | | | |
| | pathway) | | | | | | | | | |
| PWY-7184 | pyrimidine | 94 | 0.002806 | 7.71 | 58848.19 | 82997.79 | 1.00 | 7634.11 | 6451.00 | 1.00 |
| | deoxyribonucleotides | | | | | | | | | |
| | de novo | | | | | | | | | |
| | biosynthesis I | | | | | | | | | |
| HEMESYN2- | heme | 95 | 0.002799 | 6.64 | 29068.34 | 65254.98 | 1.00 | 4380.26 | 5254.43 | 1.00 |
| PWY | biosynthesis II | | | | | | | | | |
| | (anaerobic) | | | | | | | | | |
| PWY-6182 | superpathway | 96 | 0.002709 | 2.17 | 489.00 | 6089.83 | 0.70 | 225.23 | 534.94 | 0.78 |
| | of salicylate | | | | | | | | | |
| | degradation | | | | | | | | | |
| PWY-6906 | chitin | 97 | 0.002685 | 0.83 | 84.08 | 256.80 | 0.69 | 100.73 | 367.28 | 0.65 |
| | derivatives | | | | | | | | | |
| | degradation | | | | | | | | | |
| PWY-3781 | aerobic | 98 | 0.002668 | 3.39 | 4627.12 | 22834.50 | 0.87 | 1366.89 | 6073.16 | 0.89 |
| | respiration I | | | | | | | | | |
| | (cytochrome c) | | | | | | | | | |
| ARGORNPROST- | arginine, | 99 | 0.002656 | 3.11 | 5675.54 | 8978.45 | 1.00 | 1826.62 | 2647.27 | 1.00 |
| PWY | ornithine and | | | | | | | | | |
| | proline | | | | | | | | | |
| | interconversion | | | | | | | | | |
| POLYISOPRENSYN- | polyisoprenoid | 100 | 0.002655 | 9.57 | 92086.27 | 118040.44 | 1.00 | 9617.67 | 7682.53 | 1.00 |
| PWY | biosynthesis | | | | | | | | | |
| | (E. coli) | | | | | | | | | |

IBD = Inflammatory Bowel Disease;
CDI = Clostridiodes difficile Infection;
RFFR = Random Forest Feature Rank;
# = Rank;
MDA = Mean Decrease Accuracy;
FC = Fold Change;
M = Mean;
SD = Standard Deviation;
DP = Detection Prevalence;

TABLE 4
IBD (Ulcerative Colitis) vs Control delineation features - (metabolic pathway)

| MetaCyc | Pathway | RFFR | | FC | Abundance (UC) | | | Abundance (Ctrl) | | |
|---|---|---|---|---|---|---|---|---|---|---|
| Pathway ID | description | # | MDA | UC vs Ctrl | M | SD | DP | M | SD | DP |
| PWY-5484 | glycolysis II | 1 | 0.013468 | 5.34 | 137874.28 | 163049.92 | 1.00 | 25795.46 | 58601.44 | 1.00 |
| | (from fructose | | | | | | | | | |
| | 6-phosphate) | | | | | | | | | |
| PWY-7220 | adenosine | 2 | 0.012501 | 5.02 | 187150.53 | 217714.47 | 1.00 | 37306.88 | 84124.81 | 1.00 |
| | deoxyribonucleotides | | | | | | | | | |
| | de novo | | | | | | | | | |
| | biosynthesis II | | | | | | | | | |
| DTDPRHAMSYN- | dTDP-L- | 3 | 0.012165 | 5.39 | 189125.72 | 234912.05 | 1.00 | 35109.82 | 75193.40 | 1.00 |
| PWY | rhamnose | | | | | | | | | |
| | biosynthesis I | | | | | | | | | |
| GLUCONEO- | gluconeogenesis I | 4 | 0.011578 | 5.19 | 143219.37 | 167868.16 | 1.00 | 27620.28 | 63540.87 | 1.00 |
| PWY | | | | | | | | | | |
| PWY-7222 | guanosine | 5 | 0.010944 | 5.02 | 187150.53 | 217714.47 | 1.00 | 37306.88 | 84124.81 | 1.00 |
| | deoxyribonucleotides | | | | | | | | | |
| | de novo | | | | | | | | | |
| | biosynthesis II | | | | | | | | | |
| PWY-6737 | starch | 6 | 0.010427 | 4.97 | 216898.43 | 245631.54 | 1.00 | 43660.03 | 89100.84 | 1.00 |
| | degradation V | | | | | | | | | |
| GLYCOLYSIS | glycolysis I | 7 | 0.009969 | 5.00 | 151732.58 | 173723.01 | 1.00 | 30345.44 | 65251.30 | 1.00 |
| | (from glucose | | | | | | | | | |
| | 6-phosphate) | | | | | | | | | |
| FERMENTATION- | mixed acid | 8 | 0.009004 | 4.81 | 87462.44 | 101627.24 | 1.00 | 18196.38 | 37540.58 | 1.00 |
| PWY | fermentation | | | | | | | | | |
| RIBOSYN | flavin | 9 | 0.007731 | 4.85 | 124769.36 | 141377.01 | 1.00 | 25710.38 | 56666.93 | 1.00 |
| 2-PWY | biosynthesis I | | | | | | | | | |
| | (bacteria and | | | | | | | | | |
| | plants) | | | | | | | | | |
| ANAEROFRUCAT- | homolactic | 10 | 0.007429 | 4.67 | 121247.42 | 135856.30 | 1.00 | 25969.53 | 52349.57 | 1.00 |
| PWY | fermentation | | | | | | | | | |
| POLYISOPRENSYN- | polyisoprenoid | 11 | 0.007394 | 5.57 | 103349.13 | 126047.74 | 1.00 | 18570.50 | 48260.04 | 1.00 |
| PWY | biosynthesis | | | | | | | | | |
| | (E. coli) | | | | | | | | | |
| PWY-6969 | TCA cycle V (2- | 12 | 0.007367 | 6.00 | 123707.97 | 159065.04 | 1.00 | 20619.10 | 56313.80 | 1.00 |
| | oxoglutarate:ferredoxin | | | | | | | | | |
| | oxidoreductase) | | | | | | | | | |
| THRESYN- | superpathway | 13 | 0.006838 | 4.68 | 158299.07 | 173526.15 | 1.00 | 33804.17 | 69499.08 | 1.00 |
| PWY | of L-threonine | | | | | | | | | |
| | biosynthesis | | | | | | | | | |
| PWY-7221 | guanosine | 14 | 0.006629 | 4.66 | 155880.21 | 170218.31 | 1.00 | 33485.60 | 67311.36 | 1.00 |
| | ribonucleotides | | | | | | | | | |
| | de novo | | | | | | | | | |
| | biosynthesis | | | | | | | | | |
| PENTOSE-P- | pentose | 15 | 0.006569 | 6.35 | 94692.24 | 125567.44 | 1.00 | 14915.57 | 40468.42 | 1.00 |
| PWY | phosphate | | | | | | | | | |
| | pathway | | | | | | | | | |
| PWY-5973 | cis-vaccenate | 16 | 0.006501 | 4.80 | 180359.77 | 202158.96 | 1.00 | 37597.57 | 77633.61 | 1.00 |
| | biosynthesis | | | | | | | | | |
| PWY-5097 | L-lysine | 17 | 0.006420 | 4.52 | 163088.19 | 175254.73 | 1.00 | 36065.98 | 71069.75 | 1.00 |
| | biosynthesis VI | | | | | | | | | |
| PWY-5971 | palmitate | 18 | 0.006350 | 5.13 | 66234.32 | 96843.59 | 1.00 | 12902.75 | 46991.68 | 1.00 |
| | biosynthesis II | | | | | | | | | |
| | (bacteria and | | | | | | | | | |
| | plants) | | | | | | | | | |
| HISTSYN- | L-histidine | 19 | 0.006209 | 4.69 | 143551.66 | 158396.25 | 1.00 | 30588.45 | 60932.50 | 1.00 |
| PWY | biosynthesis | | | | | | | | | |
| PWY-7200 | superpathway | 20 | 0.005995 | 4.91 | 64690.73 | 76056.16 | 1.00 | 13163.34 | 32475.36 | 1.00 |
| | of pyrimidine | | | | | | | | | |
| | deoxyribonucleoside | | | | | | | | | |
| | salvage | | | | | | | | | |
| PWY-6126 | superpathway | 21 | 0.005993 | 4.68 | 166511.68 | 182033.40 | 1.00 | 35557.29 | 73657.90 | 1.00 |
| | of adenosine | | | | | | | | | |
| | nucleotides de | | | | | | | | | |
| | novo | | | | | | | | | |
| | biosynthesis II | | | | | | | | | |
| PWY0- | anhydromuropeptides | 22 | 0.005971 | 4.76 | 49694.18 | 70536.52 | 1.00 | 10433.27 | 26429.41 | 1.00 |
| 1261 | recycling | | | | | | | | | |
| TRNA- | tRNA charging | 23 | 0.005964 | 4.50 | 144826.75 | 155388.02 | 1.00 | 32210.70 | 64009.96 | 1.00 |
| CHARGING-PWY | | | | | | | | | | |
| PWY-5101 | L-isoleucine | 24 | 0.005927 | 4.50 | 183959.02 | 197410.19 | 1.00 | 40907.22 | 78658.73 | 1.00 |
| | biosynthesis II | | | | | | | | | |
| PYRIDNUCSAL- | NAD salvage | 25 | 0.005849 | 4.67 | 128877.76 | 142195.58 | 1.00 | 27573.47 | 56793.93 | 1.00 |
| PWY | pathway I | | | | | | | | | |
| PWY-6471 | peptidoglycan | 26 | 0.005815 | 3.66 | 56783.16 | 62569.90 | 1.00 | 15517.50 | 26202.50 | 1.00 |
| | biosynthesis IV | | | | | | | | | |
| | (Enterococcus | | | | | | | | | |
| | faecium) | | | | | | | | | |
| PWY-7199 | pyrimidine | 27 | 0.005786 | 5.14 | 105140.04 | 125489.97 | 1.00 | 20465.73 | 48211.04 | 1.00 |
| | deoxyribonucleosides | | | | | | | | | |
| | salvage | | | | | | | | | |
| PWY-6901 | superpathway | 28 | 0.005719 | 5.79 | 93437.75 | 115918.50 | 1.00 | 16138.44 | 42305.62 | 1.00 |
| | of glucose and | | | | | | | | | |
| | xylose | | | | | | | | | |
| | degradation | | | | | | | | | |
| PWY-6612 | superpathway of | 29 | 0.005486 | 4.77 | 113726.13 | 128397.78 | 1.00 | 23842.31 | 53794.55 | 1.00 |
| | tetrahydrofolate | | | | | | | | | |
| | biosynthesis | | | | | | | | | |
| PWY-7229 | superpathway | 30 | 0.005395 | 4.68 | 174435.87 | 190236.65 | 1.00 | 37269.82 | 76809.15 | 1.00 |
| | of adenosine | | | | | | | | | |
| | nucleotides de | | | | | | | | | |
| | novo | | | | | | | | | |
| | biosynthesis I | | | | | | | | | |
| PWY-5265 | peptidoglycan | 31 | 0.005341 | 3.50 | 958.53 | 3382.00 | 0.89 | 273.60 | 1410.22 | 0.80 |
| | biosynthesis II | | | | | | | | | |
| | (staphylococci) | | | | | | | | | |
| BRANCHED- | superpathway | 32 | 0.005160 | 4.51 | 161266.67 | 173339.12 | 1.00 | 35733.57 | 69393.77 | 1.00 |
| CHAIN-AA- | of branched | | | | | | | | | |
| SYN-PWY | amino acid | | | | | | | | | |
| | biosynthesis | | | | | | | | | |
| PWY-5659 | GDP-mannose | 33 | 0.005106 | 5.15 | 111981.31 | 134669.98 | 1.00 | 21758.46 | 52092.19 | 1.00 |
| | biosynthesis | | | | | | | | | |
| PWY-6123 | inosine-5′- | 34 | 0.005059 | 4.64 | 151796.28 | 166324.65 | 1.00 | 32731.91 | 67616.56 | 1.00 |
| | phosphate | | | | | | | | | |
| | biosynthesis I | | | | | | | | | |
| PWY-3001 | superpathway | 35 | 0.005059 | 4.62 | 161565.25 | 175615.84 | 1.00 | 34993.92 | 70100.63 | 1.00 |
| | of L-isoleucine | | | | | | | | | |
| | biosynthesis I | | | | | | | | | |
| PWY-2942 | L-lysine | 36 | 0.004924 | 4.56 | 166999.88 | 179907.40 | 1.00 | 36593.12 | 72497.97 | 1.00 |
| | biosynthesis III | | | | | | | | | |
| PWY-5695 | urate | 37 | 0.004904 | 5.09 | 129768.00 | 151875.33 | 1.00 | 25478.81 | 61554.77 | 1.00 |
| | biosynthesis/ | | | | | | | | | |
| | inosine 5′- | | | | | | | | | |
| | phosphate | | | | | | | | | |
| | degradation | | | | | | | | | |
| PWY-7456 | mannan | 38 | 0.004826 | 6.37 | 59391.38 | 79508.41 | 0.99 | 9327.98 | 25079.05 | 0.99 |
| | degradation | | | | | | | | | |
| FOLSYN- | superpathway of | 39 | 0.004747 | 4.80 | 125689.08 | 141712.00 | 1.00 | 26199.43 | 57898.05 | 1.00 |
| PWY | tetrahydrofolate | | | | | | | | | |
| | biosynthesis | | | | | | | | | |
| | and salvage | | | | | | | | | |
| VALDEG- | L-valine | 40 | 0.004739 | 5.79 | 48.88 | 214.52 | 0.39 | 8.44 | 70.49 | 0.24 |
| PWY | degradation I | | | | | | | | | |
| ANAGLYCOLYSIS- | glycolysis III | 41 | 0.004500 | 4.52 | 173969.55 | 188915.73 | 1.00 | 38524.25 | 76074.72 | 1.00 |
| PWY | (from glucose) | | | | | | | | | |
| PWY-5121 | superpathway of | 42 | 0.004451 | 4.59 | 145660.32 | 158877.65 | 1.00 | 31735.49 | 6396 | 1.00 |
| | geranylgeranyl | | | | | | | | | |
| | diphosphate | | | | | | | | | |
| | biosynthesis II | | | | | | | | | |
| | (via MEP) | | | | | | | | | |
| PWY-7219 | adenosine | 43 | 0.004385 | 4.56 | 168432.85 | 181595.69 | 1.00 | 36934.72 | 73745.19 | 1.00 |
| | ribonucleotides | | | | | | | | | |
| | de novo | | | | | | | | | |
| | biosynthesis | | | | | | | | | |
| TRPSYN- | L-tryptophan | 44 | 0.004322 | 4.86 | 128144.72 | 146779.35 | 1.00 | 26384.56 | 58066.86 | 1.00 |
| PWY | biosynthesis | | | | | | | | | |
| ASPASN- | superpathway | 45 | 0.004278 | 5.01 | 139280.20 | 163853.94 | 1.00 | 27802.72 | 61241.17 | 1.00 |
| PWY | of L-aspartate | | | | | | | | | |
| | and L- | | | | | | | | | |
| | asparagine | | | | | | | | | |
| | biosynthesis | | | | | | | | | |
| PWY-5103 | L-isoleucine | 46 | 0.004253 | 4.53 | 160258.88 | 172574.42 | 1.00 | 35363.64 | 68670.88 | 1.00 |
| | biosynthesis III | | | | | | | | | |
| SER-GLYSYN- | superpathway | 47 | 0.004235 | 4.56 | 143605.20 | 155090.21 | 1.00 | 31513.44 | 61945.68 | 1.00 |
| PWY | of L-serine and | | | | | | | | | |
| | glycine | | | | | | | | | |
| | biosynthesis I | | | | | | | | | |
| TCA | TCA cycle I | 48 | 0.004201 | 6.17 | 98126.13 | 130990.80 | 1.00 | 15894.91 | 44406.77 | 1.00 |
| | (prokaryotic) | | | | | | | | | |
| COA-PWY | coenzyme A | 49 | 0.004073 | 4.52 | 138903.51 | 149373.92 | 1.00 | 30734.70 | 62246.18 | 1.00 |
| | biosynthesis I | | | | | | | | | |
| PWY4FS-7 | phosphatidylglycerol | 50 | 0.004060 | 4.12 | 128337.23 | 136928.30 | 1.00 | 31156.08 | 55249.99 | 1.00 |
| | biosynthesis I | | | | | | | | | |
| | (plastidic) | | | | | | | | | |
| FASYN-ELONG- | fatty acid | 51 | 0.004052 | 5.86 | 142121.29 | 184812.05 | 1.00 | 24234.67 | 67787.32 | 1.00 |
| PWY | elongation -- | | | | | | | | | |
| | saturated | | | | | | | | | |
| PANTOSYN- | pantothenate | 52 | 0.004050 | 4.88 | 121558.14 | 138828.52 | 1.00 | 24898.21 | 53813.43 | 1.00 |
| PWY | and coenzyme | | | | | | | | | |
| | A biosynthesis I | | | | | | | | | |
| P124-PWY | Bifidobacterium | 53 | 0.003954 | 3.42 | 16833.53 | 24551.47 | 1.00 | 4916.18 | 11478.79 | 1.00 |
| | shunt | | | | | | | | | |
| P161-PWY | acetylene | 54 | 0.003925 | 3.76 | 79808.50 | 85551.86 | 1.00 | 21213.96 | 36857.71 | 1.00 |
| | degradation | | | | | | | | | |
| GALACTARDEG- | D-galactarate | 55 | 0.003924 | 3.84 | 2111.64 | 7543.61 | 1.00 | 549.35 | 4744.41 | 0.99 |
| PWY | degradation I | | | | | | | | | |
| P122-PWY | heterolactic | 56 | 0.003886 | 3.87 | 13280.01 | 19573.61 | 1.00 | 3434.87 | 7997.81 | 1.00 |
| | fermentation | | | | | | | | | |
| PEPTIDOGLYCANSYN- | peptidoglycan | 57 | 0.003861 | 4.49 | 142567.46 | 152892.70 | 1.00 | 31783.86 | 62985.20 | 1.00 |
| PWY | biosynthesis I | | | | | | | | | |
| | (meso- | | | | | | | | | |
| | diaminopimelate | | | | | | | | | |
| | containing) | | | | | | | | | |
| PWY4FS-8 | phosphatidylglycerol | 58 | 0.003841 | 4.12 | 128337.23 | 136928.30 | 1.00 | 31156.08 | 55249.99 | 1.00 |
| | biosynthesis II | | | | | | | | | |
| | (non-plastidic) | | | | | | | | | |
| 1CMET2- | N10-formyl- | 59 | 0.003810 | 4.67 | 138685.80 | 152817.72 | 1.00 | 29667.15 | 61241.36 | 1.00 |
| PWY | tetrahydrofolate | | | | | | | | | |
| | biosynthesise | | | | | | | | | |
| PWY-7539 | 6-hydroxymethyl- | 60 | 0.003796 | 4.99 | 119874.08 | 140866.61 | 1.00 | 24023.34 | 57775.64 | 1.00 |
| | dihydropterin | | | | | | | | | |
| | diphosphate | | | | | | | | | |
| | biosynthesis III | | | | | | | | | |
| | (Chlamydia) | | | | | | | | | |
| VALSYN- | L-valine | 61 | 0.003760 | 4.47 | 169183.59 | 181085.81 | 1.00 | 37825.61 | 73282.50 | 1.00 |
| PWY | biosynthesis | | | | | | | | | |
| PWY-5686 | UMP | 62 | 0.003728 | 4.52 | 163185.12 | 174833.16 | 1.00 | 36137.39 | 71110.33 | 1.00 |
| | biosynthesis | | | | | | | | | |
| PWY-6151 | S-adenosy1-L- | 63 | 0.003710 | 4.32 | 118471.43 | 124314.40 | 1.00 | 27430.06 | 52342.86 | 1.00 |
| | methionine | | | | | | | | | |
| | cycle I | | | | | | | | | |
| PWY-7663 | gondoate | 64 | 0.003678 | 4.94 | 201476.18 | 230015.10 | 1.00 | 40750.28 | 88888.09 | 1.00 |
| | biosynthesis | | | | | | | | | |
| | (anaerobic) | | | | | | | | | |
| PWY-6163 | chorismate | 65 | 0.003673 | 4.46 | 152411.28 | 162586.14 | 1.00 | 34156.88 | 67511.88 | 1.00 |
| | biosynthesis | | | | | | | | | |
| | from 3- | | | | | | | | | |
| | dehydroquinate | | | | | | | | | |
| PWY-6703 | preQ0 | 66 | 0.003672 | 5.41 | 90603.31 | 113287.04 | 1.00 | 16750.81 | 43567.02 | 1.00 |
| | biosynthesis | | | | | | | | | |
| PWY-6897 | thiamin | 67 | 0.003672 | 4.61 | 119488.56 | 134963.50 | 1.00 | 25927.29 | 55804.92 | 1.00 |
| | salvage II | | | | | | | | | |
| UDPNAGSYN- | UDP-N-acetyl- | 68 | 0.003670 | 4.04 | 71406.12 | 80670.82 | 1.00 | 17690.50 | 33505.78 | 1.00 |
| PWY | D-glucosamine | | | | | | | | | |
| | biosynthesis I | | | | | | | | | |
| GLUCARGALACTSUPER- | superpathway | 69 | 0.003637 | 3.84 | 2111.64 | 7543.61 | 1.00 | 549.35 | 4744.41 | 0.99 |
| PWY | of D-glucarate | | | | | | | | | |
| | and D- | | | | | | | | | |
| | galactarate | | | | | | | | | |
| | degradation | | | | | | | | | |
| PWY-5384 | sucrose | 70 | 0.003626 | 4.00 | 59513.26 | 72971.49 | 1.00 | 14862.20 | 23647.49 | 1.00 |
| | degradation IV | | | | | | | | | |
| | (sucrose | | | | | | | | | |
| | phosphorylase) | | | | | | | | | |
| PWY-6386 | UDP-N- | 71 | 0.003607 | 4.48 | 143589.59 | 154313.45 | 1.00 | 32031.44 | 63604.98 | 1.00 |
| | acetylmuramoy | | | | | | | | | |
| | 1-pentapeptide | | | | | | | | | |
| | biosynthesis II | | | | | | | | | |
| | (lysine- | | | | | | | | | |
| | containing) | | | | | | | | | |
| HEMESYN2- | heme | 72 | 0.003588 | 8.97 | 23668.19 | 43786.66 | 1.00 | 2637.17 | 8694.63 | 1.00 |
| PWY | biosynthesis II | | | | | | | | | |
| | (anaerobic) | | | | | | | | | |
| GLYCOCAT- | glycogen | 73 | 0.003568 | 4.54 | 179426.89 | 196539.01 | 1.00 | 39548.53 | 74794.98 | 1.00 |
| PWY | degradation I | | | | | | | | | |
| | (bacterial) | | | | | | | | | |
| P42-PWY | incomplete | 74 | 0.003556 | 6.00 | 155257.55 | 197154.84 | 1.00 | 25857.31 | 68620.11 | 1.00 |
| | reductive TCA | | | | | | | | | |
| | cycle | | | | | | | | | |
| PYRIDNUCSYN- | NAD | 75 | 0.003545 | 4.77 | 126496.08 | 140748.67 | 1.00 | 26519.01 | 54429.15 | 1.00 |
| PWY | biosynthesis I | | | | | | | | | |
| | (from | | | | | | | | | |
| | aspartate) | | | | | | | | | |
| PWY-5913 | TCA cycle VI | 76 | 0.003542 | 6.26 | 74796.51 | 107950.49 | 1.00 | 11957.29 | 33291.95 | 1.00 |
| | (obligate | | | | | | | | | |
| | autotrophs) | | | | | | | | | |
| PWY0- | methylphosphonate | 77 | 0.003373 | 3.72 | 3150.67 | 14618.07 | 0.99 | 847.56 | 6164.16 | 0.99 |
| 1533 | degradation I | | | | | | | | | |
| PHOSLIPSYN- | superpathway of | 78 | 0.003368 | 4.25 | 143655.43 | 152059.19 | 1.00 | 33765.68 | 63738.88 | 1.00 |
| PWY | phospholipid | | | | | | | | | |
| | biosynthesis I | | | | | | | | | |
| | (bacteria) | | | | | | | | | |
| GLUCARDEG- | D-glucarate | 79 | 0.003367 | 2.84 | 2458.14 | 7559.79 | 1.00 | 865.69 | 4838.01 | 1.00 |
| PWY | degradation I | | | | | | | | | |
| NONMEVIPP- | methylerythritol | 80 | 0.003331 | 4.63 | 152085.59 | 167020.40 | 1.00 | 32817.44 | 66602.58 | 1.00 |
| PWY | phosphate | | | | | | | | | |
| | pathway I | | | | | | | | | |
| PWY-922 | mevalonate | 81 | 0.003279 | 2.94 | 1362.33 | 3932.81 | 1.00 | 463.57 | 1958.69 | 0.98 |
| | pathway I | | | | | | | | | |
| P162-PWY | L-glutamate | 82 | 0.003271 | 5.69 | 2551.20 | 7942.49 | 0.99 | 448.71 | 887.20 | 0.99 |
| | degradation V (via | | | | | | | | | |
| | hydroxyglutarate) | | | | | | | | | |
| PWY0-845 | superpathway | 83 | 0.003260 | 7.62 | 72147.89 | 105611.49 | 1.00 | 9473.82 | 32432.50 | 1.00 |
| | of pyridoxal 5′- | | | | | | | | | |
| | phosphate | | | | | | | | | |
| | biosynthesis | | | | | | | | | |
| | and salvage | | | | | | | | | |
| PANTO- | phosphopantothenate | 84 | 0.003239 | 4.99 | 116332.92 | 136488.66 | 1.00 | 23307.92 | 51652.13 | 1.00 |
| PWY | biosynthesis I | | | | | | | | | |
| PWY490-3 | nitrate | 85 | 0.003228 | 2.86 | 20420.91 | 23998.68 | 1.00 | 7137.14 | 15395.49 | 1.00 |
| | reduction VI | | | | | | | | | |
| | (assimilatory) | | | | | | | | | |
| P163-PWY | L-lysine | 86 | 0.003154 | 5.33 | 3410.33 | 6556.32 | 0.99 | 639.47 | 1586.14 | 0.99 |
| | fermentation to | | | | | | | | | |
| | acetate and | | | | | | | | | |
| | butanoate | | | | | | | | | |
| PWY-7371 | 1,4-dihydroxy- | 87 | 0.003138 | 4.68 | 4501.75 | 8592.10 | 0.97 | 962.58 | 2626.82 | 0.98 |
| | 6-naphthoate | | | | | | | | | |
| | biosynthesis II | | | | | | | | | |
| OANTIGEN- | O-antigen | 88 | 0.003093 | 4.26 | 93156.54 | 102747.40 | 1.00 | 21891.17 | 41689.77 | 1.00 |
| PWY | building blocks | | | | | | | | | |
| | biosynthesis | | | | | | | | | |
| | (E. coli) | | | | | | | | | |
| PWY-7374 | 1,4-dihydroxy- | 89 | 0.003088 | 4.99 | 3178.58 | 5725.74 | 0.93 | 637.49 | 1685.39 | 0.94 |
| | 6-naphthoate | | | | | | | | | |
| | biosynthesis I | | | | | | | | | |
| THISYN- | superpathway | 90 | 0.003066 | 5.52 | 109721.45 | 138174.15 | 1.00 | 19862.69 | 54220.75 | 1.00 |
| PWY | of thiamin | | | | | | | | | |
| | diphosphate | | | | | | | | | |
| | biosynthesis I | | | | | | | | | |
| PWY-7242 | D-fructuronate | 91 | 0.003064 | 5.23 | 116772.26 | 144256.32 | 1.00 | 22323.68 | 52697.65 | 1.00 |
| | degradation | | | | | | | | | |
| PWY-6125 | superpathway | 92 | 0.003007 | 5.59 | 104612.32 | 135029.70 | 1.00 | 18719.56 | 49663.94 | 1.00 |
| | of guanosine | | | | | | | | | |
| | nucleotides de | | | | | | | | | |
| | novo | | | | | | | | | |
| | biosynthesis II | | | | | | | | | |
| PWY-7013 | L-1,2- | 93 | 0.002955 | 3.61 | 5596.39 | 19114.27 | 1.00 | 1548.37 | 13949.03 | 1.00 |
| | propanediol | | | | | | | | | |
| | degradation | | | | | | | | | |
| PWY-6572 | chondroitin | 94 | 0.002934 | 10.46 | 50801.13 | 99501.47 | 0.99 | 4856.97 | 19285.21 | 0.99 |
| | sulfate | | | | | | | | | |
| | degradation I | | | | | | | | | |
| | (bacterial) | | | | | | | | | |
| P221-PWY | octane | 95 | 0.002919 | 2.63 | 5790.85 | 11690.85 | 0.99 | 2201.27 | 4989.83 | 0.99 |
| | oxidation | | | | | | | | | |
| PWY-6507 | 4-deoxy-L- | 96 | 0.002917 | 6.02 | 104716.90 | 139954.39 | 1.00 | 17403.45 | 48947.31 | 1.00 |
| | threo-hex-4- | | | | | | | | | |
| | enopyranuronate | | | | | | | | | |
| | degradation | | | | | | | | | |
| PWY-6641 | superpathway | 97 | 0.002909 | 3.95 | 286.50 | 782.30 | 0.53 | 72.45 | 219.02 | 0.56 |
| | of sulfolactate | | | | | | | | | |
| | degradation | | | | | | | | | |
| PWY-5667 | CDP- | 98 | 0.002901 | 4.38 | 160781.08 | 173346.52 | 1.00 | 36676.06 | 68483.69 | 1.00 |
| | diacylglycerol | | | | | | | | | |
| | biosynthesis I | | | | | | | | | |
| PWY-5910 | superpathway of | 99 | 0.002886 | 2.97 | 1859.35 | 5098.98 | 1.00 | 625.10 | 2449.67 | 0.98 |
| | geranylgeranyl | | | | | | | | | |
| | diphosphate | | | | | | | | | |
| | biosynthesis I | | | | | | | | | |
| | (via mevalonate) | | | | | | | | | |
| PWY-7208 | superpathway | 100 | 0.002847 | 4.50 | 164374.02 | 176488.98 | 1.00 | 36522.63 | 73296.77 | 1.00 |
| | of pyrimidine | | | | | | | | | |
| | nucleobases | | | | | | | | | |
| | salvage | | | | | | | | | |

IBD = Inflammatory Bowel Disease;
UC = Ulcerative Colitis;
Ctrl = Control;
RFFR = Random Forest Feature Rank;
# = Rank;
MDA = Mean Decrease Accuracy;
FC = Fold Change;
M = Mean;
SD = Standard Deviation;
DP = Detection Prevalence;

TABLE 5
IBD (Crohn's Disease) vs Control delineation features - (metabolic pathway)

| MetaCyc | Pathway | RFFR | | FC | Abundance (CD) | | | Abundance (Ctrl) | | |
|---|---|---|---|---|---|---|---|---|---|---|
| Pathway ID | description | # | MDA | CD vs Ctrl | M | SD | DP | M | SD | DP |
| P164-PWY | purine | 1 | 0.016694 | 4.75 | 39670.34 | 67177.84 | 1.00 | 8351.71 | 14822.92 | 1.00 |
| | nucleobases | | | | | | | | | |
| | degradation I | | | | | | | | | |
| | (anaerobic) | | | | | | | | | |
| HEMESYN2- | heme | 2 | 0.016193 | 13.48 | 35555.30 | 83683.62 | 1.00 | 2637.17 | 8694.63 | 1.00 |
| PWY | biosynthesis II | | | | | | | | | |
| | (anaerobic) | | | | | | | | | |
| PWY-6608 | guanosine | 3 | 0.013893 | 5.35 | 57468.79 | 97578.54 | 1.00 | 10750.82 | 21503.38 | 1.00 |
| | nucleotides | | | | | | | | | |
| | degradation III | | | | | | | | | |
| SALVADEHYPOX- | adenosine | 4 | 0.013769 | 6.02 | 44006.85 | 77885.11 | 1.00 | 7304.37 | 11647.54 | 1.00 |
| PWY | nucleotides | | | | | | | | | |
| | degradation II | | | | | | | | | |
| GALACTARDEG- | D-galactarate | 5 | 0.013575 | 13.28 | 7296.20 | 30138.69 | 0.99 | 549.35 | 4744.41 | 0.99 |
| PWY | degradation I | | | | | | | | | |
| FUC-RHAMCAT- | superpathway | 6 | 0.013508 | 4.49 | 30253.56 | 45999.22 | 1.00 | 6741.81 | 13553.66 | 1.00 |
| PWY | of fucose and | | | | | | | | | |
| | rhamnose | | | | | | | | | |
| | degradation | | | | | | | | | |
| PWY-7013 | L-1,2- | 7 | 0.012895 | 6.93 | 10736.01 | 40305.80 | 0.99 | 1548.37 | 13949.03 | 1.00 |
| | propanediol | | | | | | | | | |
| | degradation | | | | | | | | | |
| PWY-6749 | CMP- | 8 | 0.012567 | 1.59 | 9692.48 | 20449.22 | 0.99 | 6084.38 | 9079.26 | 1.00 |
| | legionaminate | | | | | | | | | |
| | biosynthesis I | | | | | | | | | |
| GLUCARGALACTSUPER- | superpathway | 9 | 0.011496 | 13.28 | 7296.20 | 30138.69 | 0.99 | 549.35 | 4744.41 | 0.99 |
| PWY | of D-glucarate | | | | | | | | | |
| | and D- | | | | | | | | | |
| | galactarate | | | | | | | | | |
| | degradation | | | | | | | | | |
| TRPSYN- | L-tryptophan | 10 | 0.009349 | 3.78 | 99634.26 | 122465.54 | 1.00 | 26384.56 | 58066.86 | 1.00 |
| PWY | biosynthesis | | | | | | | | | |
| PWY-6147 | 6-hydroxymethyl- | 11 | 0.008923 | 4.02 | 95540.73 | 120570.43 | 1.00 | 23773.45 | 55311.45 | 1.00 |
| | dihydropterin | | | | | | | | | |
| | diphosphate | | | | | | | | | |
| | biosynthesis I | | | | | | | | | |
| PWY-6353 | purine | 12 | 0.008566 | 5.26 | 47585.56 | 77325.28 | 1.00 | 9044.31 | 15206.57 | 1.00 |
| | nucleotides | | | | | | | | | |
| | degradation II | | | | | | | | | |
| | (aerobic) | | | | | | | | | |
| P125-PWY | superpathway | 13 | 0.008003 | 14.09 | 3230.07 | 12327.63 | 1.00 | 229.19 | 592.66 | 0.99 |
| | of (R,R)- | | | | | | | | | |
| | butanediol | | | | | | | | | |
| | biosynthesis | | | | | | | | | |
| GLUCUROCAT- | superpathway | 14 | 0.007973 | 5.11 | 59892.43 | 105510.95 | 1.00 | 11711.24 | 23892.96 | 1.00 |
| PWY | of &beta;-D- | | | | | | | | | |
| | glucuronide | | | | | | | | | |
| | and D- | | | | | | | | | |
| | glucuronate | | | | | | | | | |
| | degradation | | | | | | | | | |
| PWY-7220 | adenosine | 15 | 0.007777 | 4.11 | 153483.18 | 196473.92 | 1.00 | 37306.88 | 84124.81 | 1.00 |
| | deoxyribonucleotides | | | | | | | | | |
| | de novo | | | | | | | | | |
| | biosynthesis II | | | | | | | | | |
| GALACT- | superpathway | 16 | 0.007634 | 4.43 | 52193.98 | 72901.05 | 1.00 | 11783.19 | 25094.72 | 1.00 |
| GLUCUROCAT- | of hexuronide | | | | | | | | | |
| PWY | and hexuronate | | | | | | | | | |
| | degradation | | | | | | | | | |
| GLUCARDEG- | D-glucarate | 17 | 0.007549 | 9.07 | 7850.61 | 30420.23 | 0.99 | 865.69 | 4838.01 | 1.00 |
| PWY | degradation I | | | | | | | | | |
| PWY-7222 | guanosine | 18 | 0.007456 | 4.11 | 153483.18 | 196473.92 | 1.00 | 37306.88 | 84124.81 | 1.00 |
| | deoxyribonucleotides | | | | | | | | | |
| | de novo | | | | | | | | | |
| | biosynthesis II | | | | | | | | | |
| PENTOSE-P- | pentose | 19 | 0.007329 | 5.59 | 83420.89 | 130091.59 | 1.00 | 14915.57 | 40468.42 | 1.00 |
| PWY | phosphate | | | | | | | | | |
| | pathway | | | | | | | | | |
| PWY-7539 | 6-hydroxymethyl- | 20 | 0.007035 | 3.97 | 95422.67 | 118360.58 | 1.00 | 24023.34 | 57775.64 | 1.00 |
| | dihydropterin | | | | | | | | | |
| | diphosphate | | | | | | | | | |
| | biosynthesis III | | | | | | | | | |
| | (Chlamydia) | | | | | | | | | |
| PWY-6891 | thiazole | 21 | 0.006703 | 6.31 | 37674.05 | 78490.10 | 1.00 | 5971.37 | 17637.92 | 1.00 |
| | biosynthesis II | | | | | | | | | |
| | (Bacillus) | | | | | | | | | |
| PWY-7663 | gondoate | 22 | 0.006638 | 3.76 | 153397.37 | 191013.74 | 1.00 | 40750.28 | 88888.09 | 1.00 |
| | biosynthesis | | | | | | | | | |
| | (anaerobic) | | | | | | | | | |
| GLUCONEO- | gluconeogenesis I | 23 | 0.006519 | 4.10 | 113287.39 | 150720.86 | 1.00 | 27620.28 | 63540.87 | 1.00 |
| PWY | | | | | | | | | | |
| HISDEG- | L-histidine | 24 | 0.006455 | 5.67 | 51973.76 | 86358.88 | 1.00 | 9158.46 | 31562.90 | 1.00 |
| PWY | degradation I | | | | | | | | | |
| PWY-6263 | superpathway | 25 | 0.006421 | 2.02 | 4234.14 | 11042.21 | 0.93 | 2100.71 | 5484.38 | 0.98 |
| | of menaquinol- | | | | | | | | | |
| | 8 biosynthesis II | | | | | | | | | |
| FUCCAT- | fucose | 26 | 0.006342 | 4.50 | 31323.36 | 51124.44 | 1.00 | 6953.38 | 12213.82 | 1.00 |
| PWY | degradation | | | | | | | | | |
| PWY0-845 | superpathway | 27 | 0.006076 | 6.19 | 58606.74 | 103927.00 | 0.99 | 9473.82 | 32432.50 | 1.00 |
| | of pyridoxal 5′- | | | | | | | | | |
| | phosphate | | | | | | | | | |
| | biosynthesis | | | | | | | | | |
| | and salvage | | | | | | | | | |
| RIBOSYN2- | flavin | 28 | 0.005982 | 3.74 | 96099.98 | 117380.33 | 1.00 | 25710.38 | 56666.93 | 1.00 |
| PWY | biosynthesis I | | | | | | | | | |
| | (bacteria and | | | | | | | | | |
| | plants) | | | | | | | | | |
| GALACTUROCAT- | D-galacturonate | 29 | 0.005915 | 4.45 | 74289.85 | 110692.53 | 1.00 | 16708.71 | 38794.58 | 1.00 |
| PWY | degradation I | | | | | | | | | |
| PWY-7371 | 1,4-dihydroxy- | 30 | 0.005862 | 2.15 | 2067.29 | 9774.12 | 0.93 | 962.58 | 2626.82 | 0.98 |
| | 6-naphthoate | | | | | | | | | |
| | biosynthesis II | | | | | | | | | |
| CALVIN- | Calvin- | 31 | 0.005669 | 3.57 | 138657.97 | 168279.01 | 1.00 | 38789.56 | 77297.80 | 1.00 |
| PWY | Benson- | | | | | | | | | |
| | Bassham cycle | | | | | | | | | |
| 1CMET2- | N10-formyl- | 32 | 0.005663 | 3.69 | 109387.84 | 133407.05 | 1.00 | 29667.15 | 61241.36 | 1.00 |
| PWY | tetrahydrofolate | | | | | | | | | |
| | biosynthesis | | | | | | | | | |
| ASPASN- | superpathway | 33 | 0.005613 | 3.82 | 106098.60 | 133809.01 | 1.00 | 27802.72 | 61241.17 | 1.00 |
| PWY | of L-aspartate | | | | | | | | | |
| | and L- | | | | | | | | | |
| | asparagine | | | | | | | | | |
| | biosynthesis | | | | | | | | | |
| PWY-7229 | superpathway | 34 | 0.005411 | 3.67 | 136856.78 | 168865.72 | 1.00 | 37269.82 | 76809.15 | 1.00 |
| | of adenosine | | | | | | | | | |
| | nucleotides de | | | | | | | | | |
| | novo | | | | | | | | | |
| | biosynthesis I | | | | | | | | | |
| PWY-5028 | L-histidine | 35 | 0.005344 | 92.77 | 10707.61 | 55776.82 | 0.88 | 115.42 | 525.00 | 0.66 |
| | degradation II | | | | | | | | | |
| POLYISOPRENSYN- | polyisoprenoid | 36 | 0.005300 | 4.23 | 78556.66 | 106197.98 | 1.00 | 18570.50 | 48260.04 | 1.00 |
| PWY | biosynthesis | | | | | | | | | |
| | (E. coli) | | | | | | | | | |
| GOLPDLCAT- | superpathway | 37 | 0.005276 | 3.78 | 1999.50 | 11011.75 | 0.99 | 528.96 | 6087.60 | 0.99 |
| PWY | of glycerol | | | | | | | | | |
| | degradation to | | | | | | | | | |
| | 1,3- | | | | | | | | | |
| | propanediol | | | | | | | | | |
| PWY-7328 | superpathway | 38 | 0.005245 | 8.02 | 36501.90 | 82385.95 | 1.00 | 4548.81 | 13394.71 | 1.00 |
| | of UDP- | | | | | | | | | |
| | glucose- | | | | | | | | | |
| | derived O- | | | | | | | | | |
| | antigen | | | | | | | | | |
| | building blocks | | | | | | | | | |
| | biosynthesis | | | | | | | | | |
| PWY-5676 | acetyl-CoA | 39 | 0.005186 | 2.69 | 14818.09 | 29450.20 | 0.99 | 5508.55 | 16400.91 | 1.00 |
| | fermentation to | | | | | | | | | |
| | butanoate II | | | | | | | | | |
| PWY-6612 | superpathway of | 40 | 0.004984 | 4.02 | 95935.61 | 125669.15 | 1.00 | 23842.31 | 53794.55 | 1.00 |
| | tetrahydrofolate | | | | | | | | | |
| | biosynthesis | | | | | | | | | |
| PWY-7237 | myo-, chiro- | 41 | 0.004951 | 7.57 | 44697.50 | 120409.47 | 1.00 | 5907.27 | 15779.62 | 1.00 |
| | and scillo- | | | | | | | | | |
| | inositol | | | | | | | | | |
| | degradation | | | | | | | | | |
| PWY-5097 | L-lysine | 42 | 0.004922 | 3.58 | 129295.28 | 158559.53 | 1.00 | 36065.98 | 71069.75 | 1.00 |
| | biosynthesis VI | | | | | | | | | |
| PWY-6471 | peptidoglycan | 43 | 0.004861 | 2.37 | 36739.48 | 63784.59 | 1.00 | 15517.50 | 26202.50 | 1.00 |
| | biosynthesis IV | | | | | | | | | |
| | (Enterococcus | | | | | | | | | |
| | faecium) | | | | | | | | | |
| P162-PWY | L-glutamate | 44 | 0.004787 | 11.01 | 4941.47 | 13423.77 | 0.98 | 448.71 | 887.20 | 0.99 |
| | degradation V | | | | | | | | | |
| | (via | | | | | | | | | |
| | hydroxyglutarate) | | | | | | | | | |
| NONOXIPENT- | pentose | 45 | 0.004705 | 3.73 | 175905.63 | 236171.09 | 1.00 | 47150.96 | 90843.66 | 1.00 |
| PWY | phosphate | | | | | | | | | |
| | pathway (non- | | | | | | | | | |
| | oxidative | | | | | | | | | |
| | branch) | | | | | | | | | |
| P381-PWY | adenosylcobalamin | 46 | 0.004651 | 1.17 | 52.08 | 349.59 | 0.34 | 44.58 | 315.65 | 0.34 |
| | biosynthesis II | | | | | | | | | |
| | (late cobalt | | | | | | | | | |
| | incorporation) | | | | | | | | | |
| THRESYN- | superpathway | 47 | 0.004638 | 3.64 | 123200.37 | 148501.98 | 1.00 | 33804.17 | 69499.08 | 1.00 |
| PWY | of L-threonine | | | | | | | | | |
| | biosynthesis | | | | | | | | | |
| PWY-6588 | pyruvate | 48 | 0.004545 | 6.92 | 24791.84 | 48898.19 | 1.00 | 3584.20 | 7857.69 | 1.00 |
| | fermentation to | | | | | | | | | |
| | acetone | | | | | | | | | |
| PWY-5005 | biotin | 49 | 0.004458 | 3.01 | 4932.46 | 14746.58 | 0.97 | 1637.01 | 6066.53 | 0.99 |
| | biosynthesis II | | | | | | | | | |
| PWY-5973 | cis-vaccenate | 50 | 0.004426 | 3.69 | 138718.68 | 175425.39 | 1.00 | 37597.57 | 77633.61 | 1.00 |
| | biosynthesis | | | | | | | | | |
| ARGORNPROST- | arginine, | 51 | 0.004411 | 2.11 | 4884.11 | 9264.15 | 1.00 | 2313.51 | 6466.68 | 1.00 |
| PWY | ornithine and | | | | | | | | | |
| | proline | | | | | | | | | |
| | interconversion | | | | | | | | | |
| PWY-6897 | thiamin | 52 | 0.004373 | 3.78 | 97899.86 | 119256.02 | 1.00 | 25927.29 | 55804.92 | 1.00 |
| | salvage II | | | | | | | | | |
| FOLSYN- | superpathwayof | 53 | 0.004333 | 3.93 | 102954.52 | 132523.76 | 1.00 | 26199.43 | 57898.05 | 1.00 |
| PWY | tetrahydrofolate | | | | | | | | | |
| | biosynthesis | | | | | | | | | |
| | and salvage | | | | | | | | | |
| PWY0- | methylphosphonate | 54 | 0.004302 | 19.74 | 16734.20 | 74852.92 | 0.99 | 847.56 | 6164.16 | 0.99 |
| 1533 | degradation I | | | | | | | | | |
| PWY-6396 | superpathway | 55 | 0.004283 | 10.30 | 2818.46 | 6448.24 | 1.00 | 273.69 | 669.20 | 0.99 |
| | of 2,3- | | | | | | | | | |
| | butanediol | | | | | | | | | |
| | biosynthesis | | | | | | | | | |
| PWY-7376 | cob(II)yrinate | 56 | 0.004263 | 2.39 | 125.18 | 1574.90 | 0.35 | 52.48 | 565.70 | 0.35 |
| | a,c-diamide | | | | | | | | | |
| | biosynthesis II | | | | | | | | | |
| | (late cobalt | | | | | | | | | |
| | incorporation) | | | | | | | | | |
| PWY-6277 | superpathway | 57 | 0.004088 | 3.47 | 118847.46 | 140173.41 | 1.00 | 34211.58 | 67927.32 | 1.00 |
| | of 5- | | | | | | | | | |
| | aminoimidazole | | | | | | | | | |
| | ribonucleotide | | | | | | | | | |
| | biosynthesis | | | | | | | | | |
| DTDPRHAMSYN- | dTDP-L- | 58 | 0.004029 | 3.57 | 125482.40 | 148314.44 | 1.00 | 35109.82 | 75193.40 | 1.00 |
| PWY | rhamnose | | | | | | | | | |
| | biosynthesis I | | | | | | | | | |
| PYRIDOXSYN- | pyridoxal 5′- | 59 | 0.004018 | 6.29 | 49561.69 | 87964.19 | 0.99 | 7874.62 | 29079.28 | 1.00 |
| PWY | phosphate | | | | | | | | | |
| | biosynthesis I | | | | | | | | | |
| PANTO- | phosphopantothenate | 60 | 0.004016 | 3.72 | 86801.13 | 108009.43 | 1.00 | 23307.92 | 51652.13 | 1.00 |
| PWY | biosynthesis I | | | | | | | | | |
| PWY-6876 | isopropanol | 61 | 0.003974 | 3.21 | 726.65 | 3973.66 | 0.85 | 226.52 | 916.56 | 0.87 |
| | biosynthesis | | | | | | | | | |
| PWY-5686 | UMP | 62 | 0.003923 | 3.51 | 127018.63 | 153797.12 | 1.00 | 36137.39 | 71110.33 | 1.00 |
| | biosynthesis | | | | | | | | | |
| PANTOSYN- | pantothenate | 63 | 0.003872 | 3.63 | 90363.19 | 110841.09 | 1.00 | 24898.21 | 53813.43 | 1.00 |
| PWY | and coenzyme | | | | | | | | | |
| | A biosynthesis I | | | | | | | | | |
| PWY-7560 | methylerythritol | 64 | 0.003859 | 3.43 | 112677.00 | 135610.11 | 1.00 | 32817.44 | 66602.58 | 1.00 |
| | phosphate | | | | | | | | | |
| | pathway II | | | | | | | | | |
| PWY-7374 | 1,4-dihydroxy- | 65 | 0.003845 | 1.64 | 1047.15 | 2383.26 | 0.88 | 637.49 | 1685.39 | 0.94 |
| | 6-naphthoate | | | | | | | | | |
| | biosynthesis I | | | | | | | | | |
| PWY-6895 | superpathway | 66 | 0.003701 | 5.23 | 53144.48 | 88992.18 | 1.00 | 10160.16 | 32128.66 | 1.00 |
| | of thiamin | | | | | | | | | |
| | diphosphate | | | | | | | | | |
| | biosynthesis II | | | | | | | | | |
| PYRIDNUCSYN- | NAD | 67 | 0.003684 | 3.51 | 92993.32 | 114472.99 | 1.00 | 26519.01 | 54429.15 | 1.00 |
| PWY | biosynthesis I | | | | | | | | | |
| | (from aspartate) | | | | | | | | | |
| PWY-6387 | UDP-N- | 68 | 0.003683 | 3.51 | 112245.75 | 133752.60 | 1.00 | 31963.32 | 63342.58 | 1.00 |
| | acetylmuramoy | | | | | | | | | |
| | 1-pentapeptide | | | | | | | | | |
| | biosynthesis I | | | | | | | | | |
| | (meso- | | | | | | | | | |
| | diaminopimelate | | | | | | | | | |
| | containing) | | | | | | | | | |
| PWY0- | ADP-L- | 69 | 0.003673 | 8.33 | 23709.16 | 65604.48 | 1.00 | 2845.86 | 10461.74 | 1.00 |
| 1241 | glycero-&beta;- | | | | | | | | | |
| | D-manno-heptose | | | | | | | | | |
| | biosynthesis | | | | | | | | | |
| PWY-5100 | pyruvate | 70 | 0.003535 | 3.53 | 120253.39 | 144453.37 | 1.00 | 34069.08 | 66777.05 | 1.00 |
| | fermentation to | | | | | | | | | |
| | acetate and | | | | | | | | | |
| | lactate II | | | | | | | | | |
| PWY0-41 | allantoin | 71 | 0.003521 | 23.89 | 5721.43 | 26198.07 | 0.99 | 239.53 | 766.40 | 0.99 |
| | degradation IV | | | | | | | | | |
| | (anaerobic) | | | | | | | | | |
| ANAGLYCOLYSIS- | glycolysis III | 72 | 0.003487 | 3.57 | 137524.04 | 168787.18 | 1.00 | 38524.25 | 76074.72 | 1.00 |
| PWY | (from glucose) | | | | | | | | | |
| RHAMCAT- | L-rhamnose | 73 | 0.003485 | 4.26 | 56913.94 | 79913.50 | 1.00 | 13374.68 | 37212.50 | 1.00 |
| PWY | degradation I | | | | | | | | | |
| GLUTORN- | L-ornithine | 74 | 0.003416 | 3.75 | 88647.53 | 113606.60 | 1.00 | 23616.52 | 46464.24 | 1.00 |
| PWY | biosynthesis | | | | | | | | | |
| TRNA- | tRNA charging | 75 | 0.003370 | 3.49 | 112518.40 | 135123.34 | 1.00 | 32210.70 | 64009.96 | 1.00 |
| CHARGING-PWY | | | | | | | | | | |
| PWY-6641 | superpathway | 76 | 0.003319 | 0.42 | 30.61 | 232.54 | 0.32 | 72.45 | 219.02 | 0.56 |
| | of sulfolactate | | | | | | | | | |
| | degradation | | | | | | | | | |
| PWY-7111 | pyruvate | 77 | 0.003284 | 3.64 | 151140.97 | 194928.89 | 1.00 | 41507.13 | 77556.59 | 1.00 |
| | fermentation to | | | | | | | | | |
| | isobutanol | | | | | | | | | |
| | (engineered) | | | | | | | | | |
| NONMEVIPP- | methylerythritol | 78 | 0.003273 | 3.43 | 112677.00 | 135610.11 | 1.00 | 32817.44 | 66602.58 | 1.00 |
| PWY | phosphate | | | | | | | | | |
| | pathway I | | | | | | | | | |
| PWY-7242 | D-fructuronate | 79 | 0.003250 | 4.42 | 98688.06 | 150425.27 | 1.00 | 22323.68 | 52697.65 | 1.00 |
| | degradation | | | | | | | | | |
| COA-PWY | coenzyme A | 80 | 0.003236 | 3.43 | 105562.92 | 124974.11 | 1.00 | 30734.70 | 62246.18 | 1.00 |
| | biosynthesis I | | | | | | | | | |
| PWY-7219 | adenosine | 81 | 0.003202 | 3.50 | 129271.94 | 155301.58 | 1.00 | 36934.72 | 73745.19 | 1.00 |
| | ribonucleotides | | | | | | | | | |
| | de novo | | | | | | | | | |
| | biosynthesis | | | | | | | | | |
| PWY-7221 | guanosine | 82 | 0.003170 | 3.60 | 120689.24 | 147567.92 | 1.00 | 33485.60 | 67311.36 | 1.00 |
| | ribonucleotides | | | | | | | | | |
| | de novo | | | | | | | | | |
| | biosynthesis | | | | | | | | | |
| PWY-7456 | mannan | 83 | 0.003110 | 3.50 | 32642.66 | 51192.93 | 0.99 | 9327.98 | 25079.05 | 0.99 |
| | degradation | | | | | | | | | |
| PWY-6126 | superpathway | 84 | 0.003103 | 3.66 | 130284.17 | 161476.86 | 1.00 | 35557.29 | 73657.90 | 1.00 |
| | of adenosine | | | | | | | | | |
| | nucleotides de | | | | | | | | | |
| | novo | | | | | | | | | |
| | biosynthesis II | | | | | | | | | |
| PWY-6123 | inosine-5′- | 85 | 0.003046 | 3.50 | 114458.58 | 135960.11 | 1.00 | 32731.91 | 67616.56 | 1.00 |
| | phosphate | | | | | | | | | |
| | biosynthesis I | | | | | | | | | |
| PWY-6386 | UDP-N- | 86 | 0.003007 | 3.49 | 111808.01 | 132780.27 | 1.00 | 32031.44 | 63604.98 | 1.00 |
| | acetylmuramoyl- | | | | | | | | | |
| | pentapeptide | | | | | | | | | |
| | biosynthesis II | | | | | | | | | |
| | (lysine- | | | | | | | | | |
| | containing) | | | | | | | | | |
| PWY-6121 | 5-aminoimidazole | 87 | 0.002999 | 3.54 | 122644.71 | 146484.55 | 1.00 | 34599.61 | 69821.14 | 1.00 |
| | ribonucleotide | | | | | | | | | |
| | biosynthesis I | | | | | | | | | |
| PWY-6148 | tetrahydromethanopterin | 88 | 0.002996 | 0.39 | 136.10 | 1147.78 | 0.24 | 349.98 | 1424.19 | 0.47 |
| | biosynthesis | | | | | | | | | |
| P241-PWY | coenzyme B | 89 | 0.002972 | 0.41 | 118.44 | 1062.23 | 0.25 | 286.39 | 1179.05 | 0.48 |
| | biosynthesis | | | | | | | | | |
| DHGLUCONATE- | glucose | 90 | 0.002941 | 6.44 | 113.02 | 1654.90 | 0.22 | 17.54 | 290.23 | 0.24 |
| PYR-CAT-PWY | degradation | | | | | | | | | |
| | (oxidative) | | | | | | | | | |
| PWY-5705 | allantoin | 91 | 0.002936 | 10.96 | 9273.30 | 42040.00 | 1.00 | 846.36 | 1653.24 | 1.00 |
| | degradation to | | | | | | | | | |
| | glyoxylate III | | | | | | | | | |
| PWY-5484 | glycolysis II | 92 | 0.002893 | 4.39 | 113333.13 | 158613.50 | 1.00 | 25795.46 | 58601.44 | 1.00 |
| | (from fructose | | | | | | | | | |
| | 6-phosphate) | | | | | | | | | |
| PWY-5695 | urate biosynthesis/ | 93 | 0.002884 | 4.00 | 101818.99 | 131759.35 | 1.00 | 25478.81 | 61554.77 | 1.00 |
| | inosine 5′- | | | | | | | | | |
| | phosphate | | | | | | | | | |
| | degradation | | | | | | | | | |
| PWY-5659 | GDP-mannose | 94 | 0.002882 | 3.95 | 85855.38 | 112494.73 | 1.00 | 21758.46 | 52092.19 | 1.00 |
| | biosynthesis | | | | | | | | | |
| GLYCOLYSIS- | superpathway | 95 | 0.002856 | 6.17 | 53985.43 | 109050.32 | 1.00 | 8754.08 | 21993.00 | 1.00 |
| E-D | of glycolysis | | | | | | | | | |
| | and Entner- | | | | | | | | | |
| | Doudoroff | | | | | | | | | |
| PWY-6122 | 5-aminoimidazole | 96 | 0.002 | 3.47 | 118847.46 | 140173.41 | 1.00 | 34211.58 | 67927.32 | 1.00 |
| | ribonucleotide | | | | | | | | | |
| | biosynthesis II | | | | | | | | | |
| PWY0-1319 | CDP- | 97 | 0.002830 | 3.49 | 127913.86 | 154073.64 | 1.00 | 36676.06 | 68483.69 | 1.00 |
| | diacylglycerol | | | | | | | | | |
| | biosynthesis II | | | | | | | | | |
| PWY-5121 | superpathway of | 98 | 0.002828 | 3.35 | 106181.94 | 124526.29 | 1.00 | 31735.49 | 63961.74 | 1.00 |
| | geranylgeranyl | | | | | | | | | |
| | diphosphate | | | | | | | | | |
| | biosynthesis II | | | | | | | | | |
| | (via MEP) | | | | | | | | | |
| PWY-6892 | thiazole | 99 | 0.002813 | 3.87 | 105937.16 | 132442.65 | 1.00 | 27367.72 | 60807.66 | 1.00 |
| | biosynthesis I | | | | | | | | | |
| | (E. coli) | | | | | | | | | |
| METHGLYUT- | superpathway of | 100 | 0.002806 | 20.68 | 7581.18 | 27714.20 | 0.92 | 366.61 | 2302.30 | 0.87 |
| PWY | methylglyoxal | | | | | | | | | |
| | degradation | | | | | | | | | |

IBD = Inflammatory Bowel Disease;
CD = Crohn's Disease;
Ctrl = Control;
RFFR = Random Forest Feature Rank;
# = Rank;
MDA = Mean Decrease Accuracy;
FC = Fold Change;
M = Mean;
SD = Standard Deviation;
DP = Detection Prevalence;

TABLE 6
IBD (Crohn's Disease) vs IBD (Ulcerative Colitis) delineation features - (metabolic pathway)

| | | | | FC | | | | | | |
|---|---|---|---|---|---|---|---|---|---|---|
| MetaCyc | | | | CD | | | | | | |
| Pathway | Pathway | RFFR | | vs | Abundance (CD) | | | Abundance (UC) | | |
| ID | description | # | MDA | UC | M | SD | DP | M | SD | DP |
| PWY-6749 | CMP- | 1 | 0.025173 | 0.47 | 9692.48 | 20449.22 | 0.99 | 20493.05 | 25683.99 | 1.00 |
| | legionaminate | | | | | | | | | |
| | biosynthesis I | | | | | | | | | |
| PWY-6471 | peptidoglycan | 2 | 0.017712 | 0.65 | 36739.48 | 63784.59 | 1.00 | 56783.16 | 62569.90 | 1.00 |
| | biosynthesis | | | | | | | | | |
| | IV | | | | | | | | | |
| | (Enterococcus | | | | | | | | | |
| | faecium) | | | | | | | | | |
| PWY-7371 | 1,4-dihydroxy- | 3 | 0.010028 | 0.46 | 2067.29 | 9774.12 | 0.93 | 4501.75 | 8592.10 | 0.97 |
| | 6-naphthoate | | | | | | | | | |
| | biosynthesis II | | | | | | | | | |
| GALACTARDEG- | D-galactarate | 4 | 0.009754 | 3.46 | 7296.20 | 30138.69 | 0.99 | 2111.64 | 7543.61 | 1.00 |
| PWY | degradation I | | | | | | | | | |
| PWY-7374 | 1,4-dihydroxy- | 5 | 0.009713 | 0.33 | 1047.15 | 2383.26 | 0.88 | 3178.58 | 5725.74 | 0.93 |
| | 6-naphthoate | | | | | | | | | |
| | biosynthesis I | | | | | | | | | |
| PWY-6876 | isopropanol | 6 | 0.009252 | 1.01 | 726.65 | 3973.66 | 0.85 | 721.71 | 2449.90 | 0.93 |
| | biosynthesis | | | | | | | | | |
| ECASYN- | enterobacterial | 7 | 0.009012 | 5.81 | 16749.34 | 68566.19 | 0.93 | 2882.39 | 14532.68 | 0.92 |
| PWY | common | | | | | | | | | |
| | antigen | | | | | | | | | |
| | biosynthesis | | | | | | | | | |
| GLUCARGALACTSUPER- | superpathway | 8 | 0.008454 | 3.46 | 7296.20 | 30138.69 | 0.99 | 2111.64 | 7543.61 | 1.00 |
| PWY | of D-glucarate | | | | | | | | | |
| | and D- | | | | | | | | | |
| | galactarate | | | | | | | | | |
| | degradation | | | | | | | | | |
| PPGPPMET- | ppGpp | 9 | 0.008081 | 5.23 | 24469.33 | 82492.18 | 0.96 | 4679.44 | 21894.98 | 0.97 |
| PWY | biosynthesis | | | | | | | | | |
| PWY-6263 | superpathway | 10 | 0.008059 | 0.40 | 4234.14 | 11042.21 | 0.93 | 10596.57 | 18534.83 | 0.97 |
| | of menaquinol- | | | | | | | | | |
| | 8 biosynthesis | | | | | | | | | |
| | II | | | | | | | | | |
| P125-PWY | superpathway | 11 | 0.007435 | 4.00 | 3230.07 | 12327.63 | 1.00 | 807.17 | 3187.08 | 1.00 |
| | of (R,R)- | | | | | | | | | |
| | butanediol | | | | | | | | | |
| | biosynthesis | | | | | | | | | |
| P241-PWY | coenzyme B | 12 | 0.007167 | 0.07 | 118.44 | 1062.23 | 0.25 | 1789.01 | 14962.53 | 0.48 |
| | biosynthesis | | | | | | | | | |
| GLUCARDEG- | D-glucarate | 13 | 0.006880 | 3.19 | 7850.61 | 30420.23 | 0.99 | 2458.14 | 7559.79 | 1.00 |
| PWY | degradation I | | | | | | | | | |
| ORNARGDEG- | superpathway | 14 | 0.006817 | 6.02 | 17688.65 | 80891.73 | 0.92 | 2936.87 | 16065.76 | 0.92 |
| PWY | of L-arginine | | | | | | | | | |
| | and L- | | | | | | | | | |
| | ornithine | | | | | | | | | |
| | degradation | | | | | | | | | |
| P163-PWY | L-lysine | 15 | 0.006795 | 1.16 | 3957.48 | 14136.09 | 0.95 | 3410.33 | 6556.32 | 0.99 |
| | fermentation to | | | | | | | | | |
| | acetate and | | | | | | | | | |
| | butanoate | | | | | | | | | |
| ENTBACSYN- | enterobactin | 16 | 0.006712 | 4.74 | 19333.03 | 69230.60 | 0.93 | 4079.12 | 17264.38 | 0.94 |
| PWY | biosynthesis | | | | | | | | | |
| PWY-5676 | acetyl-CoA | 17 | 0.006685 | 0.80 | 14818.09 | 29450.20 | 0.99 | 18533.37 | 22958.79 | 1.00 |
| | fermentation to | | | | | | | | | |
| | butanoate II | | | | | | | | | |
| METHGLYUT- | superpathway | 18 | 0.006611 | 3.81 | 7581.18 | 27714.20 | 0.92 | 1989.59 | 9476.50 | 0.91 |
| PWY | of | | | | | | | | | |
| | methylglyoxal | | | | | | | | | |
| | degradation | | | | | | | | | |
| PWY-6148 | tetrahydro- | 19 | 0.006542 | 0.13 | 136.10 | 1147.78 | 0.24 | 1073.05 | 6587.85 | 0.47 |
| | methanopterin | | | | | | | | | |
| | biosynthesis | | | | | | | | | |
| AST-PWY | L-arginine | 20 | 0.006463 | 5.89 | 13603.70 | 62119.58 | 0.94 | 2310.21 | 13702.45 | 0.93 |
| | degradation II | | | | | | | | | |
| | (AST pathway) | | | | | | | | | |
| ARGDEG- | superpathway | 21 | 0.006279 | 6.02 | 17688.65 | 80891.73 | 0.92 | 2936.87 | 16065.76 | 0.92 |
| PWY | of L-arginine, | | | | | | | | | |
| | putrescine, and | | | | | | | | | |
| | 4- | | | | | | | | | |
| | aminobutanoate | | | | | | | | | |
| | degradation | | | | | | | | | |
| PWY-6349 | CDP-archaeol | 22 | 0.006072 | 0.07 | 95.29 | 837.95 | 0.24 | 1378.29 | 11158.08 | 0.47 |
| | biosynthesis | | | | | | | | | |
| ORNDEG- | superpathway | 23 | 0.006005 | 6.67 | 27281.35 | 134846.88 | 0.92 | 4090.39 | 24269.34 | 0.92 |
| PWY | of ornithine | | | | | | | | | |
| | degradation | | | | | | | | | |
| ARGORNPROST- | arginine, | 24 | 0.005927 | 0.77 | 4884.11 | 9264.15 | 1.00 | 6334.38 | 8686.73 | 1.00 |
| PWY | ornithine and | | | | | | | | | |
| | proline | | | | | | | | | |
| | interconversion | | | | | | | | | |
| PWY0- | polymyxin | 25 | 0.005909 | 6.60 | 14029.39 | 61630.04 | 0.93 | 2125.72 | 11455.28 | 0.93 |
| 1338 | resistance | | | | | | | | | |
| PWY-6654 | phosphopantothenate | 26 | 0.005889 | 0.09 | 139.12 | 1195.47 | 0.24 | 1622.79 | 12105.03 | 0.47 |
| | biosynthesis III | | | | | | | | | |
| METHANOGENESIS- | methanogenesis | 27 | 0.005595 | 0.06 | 125.20 | 1139.21 | 0.24 | 1980.08 | 16737.43 | 0.47 |
| PWY | from H2 and | | | | | | | | | |
| | CO2 | | | | | | | | | |
| PWY-7286 | 7-(3-amino-3- | 28 | 0.005543 | 0.07 | 102.64 | 926.00 | 0.24 | 1479.95 | 12108.99 | 0.47 |
| | carboxypropyl)- | | | | | | | | | |
| | wyosine | | | | | | | | | |
| | biosynthesis | | | | | | | | | |
| PWY-5028 | L-histidine | 29 | 0.005461 | 5.99 | 10707.61 | 55776.82 | 0.88 | 1786.65 | 11778.28 | 0.80 |
| | degradation II | | | | | | | | | |
| PWY-5005 | biotin | 30 | 0.005436 | 1.49 | 4932.46 | 14746.58 | 0.97 | 3318.25 | 8271.36 | 0.99 |
| | biosynthesis II | | | | | | | | | |
| PWY490-3 | nitrate | 31 | 0.005356 | 0.84 | 17051.49 | 29294.28 | 1.00 | 20420.91 | 23998.68 | 1.00 |
| | reduction VI | | | | | | | | | |
| | (assimilatory) | | | | | | | | | |
| PWY-5198 | factor 420 | 32 | 0.005320 | 0.06 | 87.93 | 791.45 | 0.30 | 1380.26 | 11688.75 | 0.50 |
| | biosynthesis | | | | | | | | | |
| GOLPDLCAT- | superpathway | 33 | 0.005305 | 1.94 | 1999.50 | 11011.75 | 0.99 | 1029.13 | 5049.35 | 0.99 |
| PWY | of glycerol | | | | | | | | | |
| | degradation to | | | | | | | | | |
| | 1,3- | | | | | | | | | |
| | propanediol | | | | | | | | | |
| PWY-5971 | palmitate | 34 | 0.005280 | 0.66 | 43665.81 | 77524.57 | 0.99 | 66234.32 | 96843.59 | 1.00 |
| | biosynthesis II | | | | | | | | | |
| | (bacteria and | | | | | | | | | |
| | plants) | | | | | | | | | |
| THREOCAT- | superpathway | 35 | 0.005256 | 4.09 | 4389.34 | 16490.46 | 0.89 | 1073.37 | 6560.79 | 0.85 |
| PWY | of L-threonine | | | | | | | | | |
| | metabolism | | | | | | | | | |
| PWY-6167 | flavin | 36 | 0.005244 | 0.11 | 226.93 | 1870.77 | 0.23 | 2148.85 | 14314.41 | 0.46 |
| | biosynthesis II | | | | | | | | | |
| | (archaea) | | | | | | | | | |
| PWY-6641 | superpathway | 37 | 0.005215 | 0.11 | 30.61 | 232.54 | 0.32 | 286.50 | 782.30 | 0.53 |
| | of sulfolactate | | | | | | | | | |
| | degradation | | | | | | | | | |
| P162-PWY | L-glutamate | 38 | 0.005157 | 1.94 | 4941.47 | 13423.77 | 0.98 | 2551.20 | 7942.49 | 0.99 |
| | degradation V | | | | | | | | | |
| | (via | | | | | | | | | |
| | hydroxyglutarate) | | | | | | | | | |
| AEROBACTINSYN- | aerobactin | 39 | 0.005127 | 9.00 | 2052.74 | 18637.22 | 0.75 | 228.13 | 1661.90 | 0.59 |
| PWY | biosynthesis | | | | | | | | | |
| PWY-7332 | superpathway | 40 | 0.005045 | 0.48 | 2593.14 | 8043.83 | 0.97 | 5350.75 | 17266.53 | 0.98 |
| | of UDP-N- | | | | | | | | | |
| | acetylglucosamine- | | | | | | | | | |
| | derived O- | | | | | | | | | |
| | antigen | | | | | | | | | |
| | building blocks | | | | | | | | | |
| | biosynthesis | | | | | | | | | |
| PWY-6350 | archaetidylinositol | 41 | 0.004967 | 0.07 | 91.42 | 810.90 | 0.24 | 1350.14 | 11043.42 | 0.47 |
| | biosynthesis | | | | | | | | | |
| PWY-6141 | archaetidylserine | 42 | 0.004906 | 0.07 | 87.56 | 782.56 | 0.23 | 1320.34 | 10921.33 | 0.46 |
| | and | | | | | | | | | |
| | archaetidyl- | | | | | | | | | |
| | ethanolamine | | | | | | | | | |
| | biosynthesis | | | | | | | | | |
| PWY-7090 | UDP-2,3- | 43 | 0.004854 | 0.69 | 261.32 | 1550.59 | 0.90 | 380.07 | 1053.00 | 0.96 |
| | diacetamido- | | | | | | | | | |
| | 2,3-dideoxy- | | | | | | | | | |
| | &alpha;-D- | | | | | | | | | |
| | mannuronate | | | | | | | | | |
| | biosynthesis | | | | | | | | | |
| P341-PWY | glycolysis V | 44 | 0.004584 | 2.49 | 469.35 | 2385.08 | 0.93 | 188.79 | 582.49 | 0.94 |
| | (Pyrococcus) | | | | | | | | | |
| PWY-6174 | mevalonate | 45 | 0.004537 | 0.07 | 100.06 | 845.39 | 0.24 | 1420.14 | 11234.68 | 0.47 |
| | pathway II | | | | | | | | | |
| | (archaea) | | | | | | | | | |
| PWY-7377 | cob(II)yrinate | 46 | 0.004479 | 1.31 | 16450.03 | 30146.81 | 1.00 | 12584.43 | 20579.09 | 1.00 |
| | a,c-diamide | | | | | | | | | |
| | biosynthesis I | | | | | | | | | |
| | (early cobalt | | | | | | | | | |
| | insertion) | | | | | | | | | |
| FUC- | superpathway | 47 | 0.004346 | 0.98 | 30253.56 | 45999.22 | 1.00 | 30982.04 | 37742.79 | 1.00 |
| RHAMCAT- | of fucose and | | | | | | | | | |
| PWY | rhamnose | | | | | | | | | |
| | degradation | | | | | | | | | |
| PWY-6071 | superpathway | 48 | 0.004303 | 7.21 | 14199.29 | 70940.66 | 0.88 | 1968.50 | 12637.00 | 0.82 |
| | of | | | | | | | | | |
| | phenylethylamine | | | | | | | | | |
| | degradation | | | | | | | | | |
| ALL- | superpathway | 49 | 0.004285 | 4.14 | 22525.78 | 78364.40 | 0.93 | 5436.31 | 21101.09 | 0.94 |
| CHORISMATE- | of chorismate | | | | | | | | | |
| PWY | metabolism | | | | | | | | | |
| P261-PWY | coenzyme M | 50 | 0.004238 | 0.07 | 99.15 | 850.63 | 0.35 | 1471.73 | 12152.92 | 0.54 |
| | biosynthesis I | | | | | | | | | |
| PWY-7456 | mannan | 51 | 0.004122 | 0.55 | 32642.66 | 51192.93 | 0.99 | 59391.38 | 79508.41 | 0.99 |
| | degradation | | | | | | | | | |
| P221-PWY | octane | 52 | 0.004029 | 0.99 | 5727.15 | 13988.98 | 0.96 | 5790.85 | 11690.85 | 0.99 |
| | oxidation | | | | | | | | | |
| P562-PWY | myo-inositol | 53 | 0.003898 | 3.41 | 16086.23 | 60565.51 | 1.00 | 4716.79 | 12464.95 | 1.00 |
| | degradation I | | | | | | | | | |
| PWY-5384 | sucrose | 54 | 0.003897 | 0.99 | 58637.96 | 107456.98 | 1.00 | 59513.26 | 72971.49 | 1.00 |
| | degradation IV | | | | | | | | | |
| | (sucrose | | | | | | | | | |
| | phosphorylase) | | | | | | | | | |
| PWY-6396 | superpathway | 55 | 0.003855 | 2.42 | 2818.46 | 6448.24 | 1.00 | 1166.35 | 4297.04 | 1.00 |
| | of 2,3- | | | | | | | | | |
| | butanediol | | | | | | | | | |
| | biosynthesis | | | | | | | | | |
| P23-PWY | reductive TCA | 56 | 0.003807 | 1.67 | 32942.69 | 71062.88 | 1.00 | 19685.12 | 36592.60 | 1.00 |
| | cycle I | | | | | | | | | |
| CODH- | reductive | 57 | 0.003796 | 0.77 | 3232.52 | 9289.40 | 0.91 | 4221.31 | 8656.55 | 0.96 |
| PWY | acetyl | | | | | | | | | |
| | coenzyme A | | | | | | | | | |
| | pathway | | | | | | | | | |
| GLYOXYLATE- | glyoxylate | 58 | 0.003767 | 4.18 | 19384.88 | 70479.27 | 0.95 | 4637.72 | 19200.94 | 0.94 |
| BYPASS | cycle | | | | | | | | | |
| PWY-6478 | GDP-D- | 59 | 0.003743 | 0.74 | 14946.39 | 23226.40 | 0.99 | 20199.10 | 33354.36 | 1.00 |
| | glycero- | | | | | | | | | |
| | &alpha;-D- | | | | | | | | | |
| | manno-heptose | | | | | | | | | |
| | biosynthesis | | | | | | | | | |
| PWY-6545 | pyrimidine | 60 | 0.003742 | 0.62 | 26564.62 | 37952.18 | 1.00 | 43085.77 | 51661.16 | 1.00 |
| | deoxyribo- | | | | | | | | | |
| | nucleotides | | | | | | | | | |
| | de novo | | | | | | | | | |
| | biosynthesis III | | | | | | | | | |
| PWY-7003 | glycerol | 61 | 0.003653 | 1.48 | 6909.54 | 16016.37 | 1.00 | 4675.89 | 9592.01 | 1.00 |
| | degradation to | | | | | | | | | |
| | butanol | | | | | | | | | |
| PWY-4984 | urea cycle | 62 | 0.003632 | 0.69 | 29111.32 | 52060.45 | 1.00 | 42029.55 | 58090.15 | 1.00 |
| PWY-7234 | inosine-5′- | 63 | 0.003582 | 1.31 | 39456.42 | 85085.98 | 1.00 | 30101.06 | 40109.85 | 1.00 |
| | phosphate | | | | | | | | | |
| | biosynthesis III | | | | | | | | | |
| PWY-7315 | dTDP-N- | 64 | 0.003562 | 1.88 | 41775.44 | 86794.48 | 1.00 | 22180.45 | 29288.33 | 1.00 |
| | acetylthomosamine | | | | | | | | | |
| | biosynthesis | | | | | | | | | |
| POLYAMINSYN3- | superpathway | 65 | 0.003518 | 0.93 | 11903.43 | 21575.86 | 0.99 | 12860.41 | 19593.62 | 1.00 |
| PWY | of polyamine | | | | | | | | | |
| | biosynthesis II | | | | | | | | | |
| PWY-7431 | aromatic | 66 | 0.003484 | 3.58 | 206.91 | 1765.80 | 0.88 | 57.87 | 95.09 | 0.92 |
| | biogenic amine | | | | | | | | | |
| | degradation | | | | | | | | | |
| | (bacteria) | | | | | | | | | |
| PWY0-321 | phenylacetate | 67 | 0.003478 | 7.12 | 14965.65 | 74424.12 | 0.89 | 2102.46 | 13279.66 | 0.83 |
| | degradation I | | | | | | | | | |
| | (aerobic) | | | | | | | | | |
| P164-PWY | purine | 68 | 0.003470 | 1.61 | 39670.34 | 67177.84 | 1.00 | 24677.59 | 28306.57 | 1.00 |
| | nucleobases | | | | | | | | | |
| | degradation I | | | | | | | | | |
| | (anaerobic) | | | | | | | | | |
| PWY-5837 | 1,4-dihydroxy- | 69 | 0.003404 | 4.01 | 21114.38 | 70449.19 | 1.00 | 5262.20 | 16610.11 | 1.00 |
| | 2-naphthoate | | | | | | | | | |
| | biosynthesis I | | | | | | | | | |
| PWY-5910 | superpathway | 70 | 0.003360 | 2.76 | 5129.11 | 20263.63 | 0.99 | 1859.35 | 5098.98 | 1.00 |
| | of | | | | | | | | | |
| | geranylgeranyl | | | | | | | | | |
| | diphosphate | | | | | | | | | |
| | biosynthesis I | | | | | | | | | |
| | (via | | | | | | | | | |
| | mevalonate) | | | | | | | | | |
| PWY-7391 | isoprene | 71 | 0.003354 | 0.13 | 28.73 | 172.00 | 0.28 | 226.77 | 1083.71 | 0.51 |
| | biosynthesis II | | | | | | | | | |
| | (engineered) | | | | | | | | | |
| PWY-5860 | superpathway | 72 | 0.003344 | 3.93 | 20493.78 | 69433.67 | 0.99 | 5208.29 | 19043.16 | 0.99 |
| | of | | | | | | | | | |
| | demethyl- | | | | | | | | | |
| | menaquinol-6 | | | | | | | | | |
| | biosynthesis I | | | | | | | | | |
| PWY-6590 | superpathway | 73 | 0.003334 | 0.86 | 12239.63 | 24318.59 | 0.99 | 14219.17 | 25405.60 | 1.00 |
| | of Clostridium | | | | | | | | | |
| | acetobutylicum | | | | | | | | | |
| | acidogenic | | | | | | | | | |
| | fermentation | | | | | | | | | |
| PWY-5863 | superpathway | 74 | 0.003318 | 3.78 | 21736.06 | 70020.79 | 1.00 | 5743.29 | 17235.41 | 1.00 |
| | of phylloquinol | | | | | | | | | |
| | biosynthesis | | | | | | | | | |
| UDPNAGSYN- | UDP-N-acetyl- | 75 | 0.003317 | 0.87 | 61879.77 | 95631.95 | 1.00 | 71406.12 | 80670.82 | 1.00 |
| PWY | D-glucosamine | | | | | | | | | |
| | biosynthesis I | | | | | | | | | |
| PWY-5862 | superpathway | 76 | 0.003312 | 3.93 | 20494.10 | 69433.58 | 0.99 | 5211.12 | 19042.72 | 0.99 |
| | of | | | | | | | | | |
| | demethyl- | | | | | | | | | |
| | menaquinol-9 | | | | | | | | | |
| | biosynthesis | | | | | | | | | |
| GLUCUROCAT- | superpathway | 77 | 0.003247 | 1.09 | 59892.43 | 105510.95 | 1.00 | 55159.45 | 71915.55 | 1.00 |
| PWY | of &beta;-D- | | | | | | | | | |
| | glucuronide | | | | | | | | | |
| | and D- | | | | | | | | | |
| | glucuronate | | | | | | | | | |
| | degradation | | | | | | | | | |
| SULFATE- | superpathway | 78 | 0.003227 | 1.11 | 44075.94 | 96361.28 | 1.00 | 39611.04 | 52690.94 | 1.00 |
| CYS- | of sulfate | | | | | | | | | |
| PWY | assimilation | | | | | | | | | |
| | and cysteine | | | | | | | | | |
| | biosynthesis | | | | | | | | | |
| PWY0-41 | allantoin | 79 | 0.003209 | 3.53 | 5721.43 | 26198.07 | 0.99 | 1622.59 | 6935.06 | 1.00 |
| | degradation IV | | | | | | | | | |
| | (anaerobic) | | | | | | | | | |
| CENTFERM- | pyruvate | 80 | 0.003188 | 0.87 | 9980.77 | 20181.96 | 0.99 | 11492.63 | 20929.21 | 1.00 |
| PWY | fermentation to | | | | | | | | | |
| | butanoate | | | | | | | | | |
| PWY-4722 | creatinine | 81 | 0.003181 | 0.84 | 32.74 | 344.87 | 0.46 | 39.08 | 115.39 | 0.63 |
| | degradation II | | | | | | | | | |
| PWY0- | methylphosphonate | 82 | 0.003141 | 5.31 | 16734.20 | 74852.92 | 0.99 | 3150.67 | 14618.07 | 0.99 |
| 1533 | degradation I | | | | | | | | | |
| PWY-6588 | pyruvate | 83 | 0.003107 | 1.53 | 24791.84 | 48898.19 | 1.00 | 16207.15 | 22849.74 | 1.00 |
| | fermentation to | | | | | | | | | |
| | acetone | | | | | | | | | |
| PWY-5022 | 4- | 84 | 0.003096 | 2.09 | 6568.44 | 14597.17 | 1.00 | 3144.44 | 6518.85 | 1.00 |
| | aminobutanoate | | | | | | | | | |
| | degradation | | | | | | | | | |
| | V | | | | | | | | | |
| GLYCOL- | superpathway | 85 | 0.003092 | 2.93 | 8884.31 | 30714.59 | 0.95 | 3030.63 | 11373.87 | 0.94 |
| GLYOXDEG- | of glycol | | | | | | | | | |
| PWY | metabolism | | | | | | | | | |
| | and | | | | | | | | | |
| | degradation | | | | | | | | | |
| PWY-5304 | superpathway | 86 | 0.003053 | 0.72 | 27360.21 | 41955.12 | 1.00 | 37846.89 | 56131.80 | 1.00 |
| | of sulfur | | | | | | | | | |
| | oxidation | | | | | | | | | |
| | (Acidianus | | | | | | | | | |
| | ambivalens) | | | | | | | | | |
| PWYO- | ADP-L- | 87 | 0.003041 | 2.23 | 23709.16 | 65604.48 | 1.00 | 10616.61 | 19216.83 | 1.00 |
| 1241 | glycero- | | | | | | | | | |
| | &beta;-D- | | | | | | | | | |
| | manno-heptose | | | | | | | | | |
| | biosynthesis | | | | | | | | | |
| PWY-1861 | formaldehyde | 88 | 0.003020 | 1.13 | 25745.51 | 60581.05 | 1.00 | 22801.52 | 40131.28 | 1.00 |
| | assimilation II | | | | | | | | | |
| | (RuMP Cycle) | | | | | | | | | |
| PWY-5861 | superpathway | 89 | 0.002959 | 3.02 | 25927.96 | 72438.40 | 1.00 | 8579.61 | 22408.94 | 1.00 |
| | of | | | | | | | | | |
| | demethyl- | | | | | | | | | |
| | menaquinol-8 | | | | | | | | | |
| | biosynthesis | | | | | | | | | |
| OANTIGEN- | O-antigen | 90 | 0.002921 | 0.77 | 72081.20 | 97443.41 | 1.00 | 93156.54 | 102747.40 | 1.00 |
| PWY | building blocks | | | | | | | | | |
| | biosynthesis | | | | | | | | | |
| | (E. coli) | | | | | | | | | |
| PWY0- | anhydromuropeptides | 91 | 0.002918 | 1.15 | 57381.54 | 130699.40 | 1.00 | 49694.18 | 70536.52 | 1.00 |
| 1261 | recycling | | | | | | | | | |
| SO4ASSIM- | sulfate | 92 | 0.002915 | 1.46 | 32883.27 | 94534.32 | 1.00 | 22540.46 | 33854.40 | 1.00 |
| PWY | reduction I | | | | | | | | | |
| | (assimilatory) | | | | | | | | | |
| LACTOSECAT- | lactose and | 93 | 0.002907 | 1.43 | 2329.83 | 5552.10 | 1.00 | 1627.19 | 2787.73 | 1.00 |
| PWY | galactose | | | | | | | | | |
| | degradation I | | | | | | | | | |
| HEME- | heme | 94 | 0.002898 | 3.82 | 17782.97 | 63997.61 | 1.00 | 4657.67 | 14559.92 | 1.00 |
| BIOSYNTHESIS- | biosynthesis I | | | | | | | | | |
| II | (aerobic) | | | | | | | | | |
| PWY-6165 | chorismate | 95 | 0.002897 | 0.67 | 175.66 | 762.40 | 0.69 | 262.88 | 754.27 | 0.80 |
| | biosynthesis II | | | | | | | | | |
| | (archaea) | | | | | | | | | |
| PWY-7013 | L-1,2- | 96 | 0.002884 | 1.92 | 10736.01 | 40305.80 | 0.99 | 5596.39 | 19114.27 | 1.00 |
| | propanediol | | | | | | | | | |
| | degradation | | | | | | | | | |
| PWY-922 | mevalonate | 97 | 0.002881 | 2.98 | 4060.72 | 17004.71 | 0.99 | 1362.33 | 3932.81 | 1.00 |
| | pathway I | | | | | | | | | |
| PWY-5838 | superpathway | 98 | 0.002861 | 2.65 | 28203.17 | 73545.04 | 1.00 | 10660.29 | 25407.65 | 1.00 |
| | of menaquinol- | | | | | | | | | |
| | 8 biosynthesis | | | | | | | | | |
| | I | | | | | | | | | |
| PWY-5154 | L-arginine | 99 | 0.002851 | 0.73 | 55076.89 | 88495.48 | 1.00 | 75404.45 | 93843.05 | 1.00 |
| | biosynthesis III | | | | | | | | | |
| | (via N-acetyl- | | | | | | | | | |
| | L-citrulline) | | | | | | | | | |
| SALVADEHYPOX- | adenosine | 100 | 0.002848 | 1.53 | 44006.85 | 77885.11 | 1.00 | 28718.36 | 35343.32 | 1.00 |
| PWY | nucleotides | | | | | | | | | |
| | degradation II | | | | | | | | | |

IBD = Inflammatory Bowel Disease; CD = Crohn's Disease; UC = Ulcerative Colitis; Ctrl = Control; RFFR = Random Forest Feature Rank; # = Rank; MDA = Mean Decrease Accuracy; FC = Fold Change; M = Mean; SD = Standard Deviation; DP = Detection Prevalence;

TABLE 7
IBS vs CDI delineation features - (metabolic pathway)

| | | | | FC | | | | | | |
|---|---|---|---|---|---|---|---|---|---|---|
| MetaCyc | | | | IBS | | | | | | |
| Pathway | Pathway | RFFR | | vs | Abundance (IBS) | | | Abundance (CDI) | | |
| ID | description | # | MDA | CDI | M | SD | DP | M | SD | DP |
| PYRIDNUCSYN- | NAD | 1 | 0.022964 | 3.10 | 28698.20 | 15283.44 | 1.00 | 9244.66 | 7697.40 | 1.00 |
| PWY | biosynthesis I | | | | | | | | | |
| | (from | | | | | | | | | |
| | aspartate) | | | | | | | | | |
| PWY-5509 | adenosylcobalamin | 2 | 0.022107 | 3.27 | 28274.99 | 16265.25 | 1.00 | 8656.86 | 7613.93 | 1.00 |
| | biosynthesis | | | | | | | | | |
| | from | | | | | | | | | |
| | cobyrinate a,c- | | | | | | | | | |
| | diamide I | | | | | | | | | |
| GLYCOGENSYNTH- | glycogen | 3 | 0.020387 | 3.41 | 38040.27 | 19979.74 | 1.00 | 11149.92 | 9865.46 | 1.00 |
| PWY | biosynthesis I | | | | | | | | | |
| | (from ADP-D- | | | | | | | | | |
| | Glucose) | | | | | | | | | |
| HISTSYN- | L-histidine | 4 | 0.020134 | 3.03 | 32757.25 | 18079.33 | 1.00 | 10804.07 | 8915.40 | 1.00 |
| PWY | biosynthesis | | | | | | | | | |
| PWY-6269 | adenosylcobalamin | 5 | 0.019140 | 3.26 | 28462.22 | 16377.77 | 1.00 | 8739.20 | 7664.91 | 1.00 |
| | salvage from | | | | | | | | | |
| | cobinamide II | | | | | | | | | |
| PYRIDNUCSAL- | NAD salvage | 6 | 0.018534 | 3.10 | 29619.59 | 15707.18 | 1.00 | 9539.35 | 8058.22 | 1.00 |
| PWY | pathway I | | | | | | | | | |
| PWY-5104 | L-isoleucine | 7 | 0.016583 | 2.94 | 40466.33 | 21816.16 | 1.00 | 13779.72 | 11296.81 | 1.00 |
| | biosynthesis | | | | | | | | | |
| | IV | | | | | | | | | |
| NONMEVIPP- | methylerythritol | 8 | 0.016184 | 2.91 | 34272.25 | 17397.13 | 1.00 | 11765.12 | 9593.11 | 1.00 |
| PWY | phosphate | | | | | | | | | |
| | pathway I | | | | | | | | | |
| PWY-6163 | chorismate | 9 | 0.016044 | 2.88 | 36411.39 | 19257.17 | 1.00 | 12651.08 | 9738.48 | 1.00 |
| | biosynthesis | | | | | | | | | |
| | from 3- | | | | | | | | | |
| | dehydroquinate | | | | | | | | | |
| PWY-6151 | S-adenosyl-L- | 10 | 0.014754 | 3.10 | 29177.44 | 15680.40 | 1.00 | 9411.61 | 7522.84 | 1.00 |
| | methionine | | | | | | | | | |
| | cycle I | | | | | | | | | |
| PWY-5101 | L-isoleucine | 11 | 0.014462 | 2.90 | 44537.47 | 25489.34 | 1.00 | 15339.54 | 12681.90 | 1.00 |
| | biosynthesis II | | | | | | | | | |
| PANTOSYN- | pantothenate | 12 | 0.014348 | 2.92 | 28076.23 | 16189.06 | 1.00 | 9601.39 | 7781.20 | 1.00 |
| PWY | and coenzyme | | | | | | | | | |
| | A biosynthesis | | | | | | | | | |
| | I | | | | | | | | | |
| PWY-5686 | UMP | 13 | 0.014067 | 2.78 | 38378.86 | 20600.14 | 1.00 | 13799.67 | 10365.94 | 1.00 |
| | biosynthesis | | | | | | | | | |
| COBALSYN- | adenosylcobalamin | 14 | 0.013167 | 3.08 | 29249.71 | 16412.21 | 1.00 | 9508.59 | 8221.80 | 1.00 |
| PWY | salvage from | | | | | | | | | |
| | cobinamide I | | | | | | | | | |
| PWY-5103 | L-isoleucine | 15 | 0.012875 | 2.89 | 38191.84 | 21780.55 | 1.00 | 13221.92 | 10973.04 | 1.00 |
| | biosynthesis III | | | | | | | | | |
| PWY-3001 | superpathway | 16 | 0.012006 | 2.78 | 36808.04 | 19082.06 | 1.00 | 13263.33 | 10722.63 | 1.00 |
| | of L-isoleucine | | | | | | | | | |
| | biosynthesis I | | | | | | | | | |
| PWY-7400 | L-arginine | 17 | 0.011770 | 3.00 | 27180.25 | 16920.29 | 1.00 | 9053.26 | 7680.28 | 1.00 |
| | biosynthesis | | | | | | | | | |
| | IV | | | | | | | | | |
| | (archaebacteria) | | | | | | | | | |
| COA-PWY | coenzyme A | 18 | 0.011481 | 2.71 | 31573.49 | 15899.61 | 1.00 | 11669.39 | 8761.12 | 1.00 |
| | biosynthesis I | | | | | | | | | |
| PWY-7560 | methylerythritol | 19 | 0.011154 | 2.91 | 34272.25 | 17397.13 | 1.00 | 11765.12 | 9593.11 | 1.00 |
| | phosphate | | | | | | | | | |
| | pathway II | | | | | | | | | |
| RIBOSYN2- | flavin | 20 | 0.010712 | 2.85 | 28641.46 | 15772.99 | 1.00 | 10066.39 | 8106.69 | 1.00 |
| PWY | biosynthesis I | | | | | | | | | |
| | (bacteria and | | | | | | | | | |
| | plants) | | | | | | | | | |
| BRANCHED- | superpathway | 21 | 0.009937 | 2.91 | 39111.81 | 23166.45 | 1.00 | 13457.08 | 11094.56 | 1.00 |
| CHAIN- | of branched | | | | | | | | | |
| AA-SYN- | amino acid | | | | | | | | | |
| PWY | biosynthesis | | | | | | | | | |
| PWY-5860 | superpathway | 22 | 0.009666 | 0.21 | 575.99 | 1472.32 | 0.96 | 2779.78 | 3938.60 | 0.98 |
| | of demethyl- | | | | | | | | | |
| | menaquinol-6 | | | | | | | | | |
| | biosynthesis I | | | | | | | | | |
| PWY-6122 | 5- | 23 | 0.009549 | 2.65 | 35079.61 | 17750.87 | 1.00 | 13242.31 | 10142.08 | 1.00 |
| | aminoimidazole | | | | | | | | | |
| | ribonucleotide | | | | | | | | | |
| | biosynthesis II | | | | | | | | | |
| P221-PWY | octane | 24 | 0.009145 | 5.50 | 1918.80 | 2782.48 | 0.94 | 348.70 | 1946.70 | 0.61 |
| | oxidation | | | | | | | | | |
| PWY-7219 | adenosine | 25 | 0.008760 | 2.69 | 38393.50 | 19673.06 | 1.00 | 14262.17 | 10745.91 | 1.00 |
| | ribonucleotides | | | | | | | | | |
| | de novo | | | | | | | | | |
| | biosynthesis | | | | | | | | | |
| PWY-7374 | 1,4-dihydroxy- | 26 | 0.008460 | 7.82 | 657.25 | 1065.52 | 0.89 | 84.04 | 289.90 | 0.50 |
| | 6-naphthoate | | | | | | | | | |
| | biosynthesis I | | | | | | | | | |
| PWY-7221 | guanosine | 27 | 0.008369 | 2.57 | 34118.96 | 16764.40 | 1.00 | 13250.25 | 10071.10 | 1.00 |
| | ribonucleotides | | | | | | | | | |
| | de novo | | | | | | | | | |
| | biosynthesis | | | | | | | | | |
| PWY-6876 | isopropanol | 28 | 0.008211 | 3.99 | 435.82 | 1957.09 | 0.98 | 109.31 | 427.04 | 0.62 |
| | biosynthesis | | | | | | | | | |
| AEROBACTINSYN- | aerobactin | 29 | 0.007987 | 0.34 | 33.89 | 199.40 | 0.34 | 100.68 | 344.92 | 0.76 |
| PWY | biosynthesis | | | | | | | | | |
| PWY-5097 | L-lysine | 30 | 0.007645 | 2.69 | 37580.67 | 18590.79 | 1.00 | 13962.57 | 10737.14 | 1.00 |
| | biosynthesis | | | | | | | | | |
| | VI | | | | | | | | | |
| PWY-5862 | superpathway | 31 | 0.007625 | 0.21 | 580.37 | 1491.91 | 0.96 | 2779.78 | 3938.60 | 0.98 |
| | of demethyl- | | | | | | | | | |
| | menaquinol-9 | | | | | | | | | |
| | biosynthesis | | | | | | | | | |
| PWY-5850 | superpathway | 32 | 0.007478 | 0.24 | 740.62 | 1757.63 | 0.96 | 3131.89 | 4208.09 | 0.98 |
| | of menaquinol- | | | | | | | | | |
| | 6 biosynthesis | | | | | | | | | |
| | I | | | | | | | | | |
| ARO-PWY | chorismate | 33 | 0.007434 | 2.87 | 36039.29 | 19742.83 | 1.00 | 12557.51 | 9830.31 | 1.00 |
| | biosynthesis I | | | | | | | | | |
| GLUTORN- | L-ornithine | 34 | 0.007347 | 3.19 | 28029.94 | 21071.03 | 1.00 | 8786.17 | 7431.15 | 1.00 |
| PWY | biosynthesis | | | | | | | | | |
| PWY-6897 | thiamin | 35 | 0.007259 | 2.95 | 29830.89 | 20011.30 | 1.00 | 10120.35 | 8107.46 | 1.00 |
| | salvage II | | | | | | | | | |
| PWY-5973 | cis-vaccenate | 36 | 0.007039 | 2.64 | 39974.77 | 20024.39 | 1.00 | 15125.38 | 11673.99 | 1.00 |
| | biosynthesis | | | | | | | | | |
| PWY0- | methylphosphonate | 37 | 0.006996 | 0.17 | 440.83 | 961.71 | 1.00 | 2590.86 | 3843.40 | 1.00 |
| 1533 | degradation I | | | | | | | | | |
| ORNDEG- | superpathway | 38 | 0.006698 | 0.14 | 259.97 | 864.26 | 0.80 | 1817.47 | 3431.71 | 0.94 |
| PWY | of ornithine | | | | | | | | | |
| | degradation | | | | | | | | | |
| SER- | superpathway | 39 | 0.006690 | 2.99 | 35169.03 | 20840.49 | 1.00 | 11750.83 | 10070.81 | 1.00 |
| GLYSYN- | of L-serine and | | | | | | | | | |
| PWY | glycine | | | | | | | | | |
| | biosynthesis I | | | | | | | | | |
| TRPSYN- | L-tryptophan | 40 | 0.006615 | 2.94 | 29664.21 | 16817.20 | 1.00 | 10083.33 | 8605.07 | 1.00 |
| PWY | biosynthesis | | | | | | | | | |
| GLUCARDEG- | D-glucarate | 41 | 0.006503 | 0.32 | 713.12 | 954.82 | 1.00 | 2209.18 | 3044.60 | 0.99 |
| PWY | degradation I | | | | | | | | | |
| COMPLETE- | superpathway | 42 | 0.006351 | 2.87 | 37695.43 | 20612.00 | 1.00 | 13134.09 | 10317.24 | 1.00 |
| ARO- | of aromatic | | | | | | | | | |
| PWY | amino acid | | | | | | | | | |
| | biosynthesis | | | | | | | | | |
| ARGSYN- | L-arginine | 43 | 0.006330 | 3.00 | 27182.21 | 17021.12 | 1.00 | 9052.44 | 7686.03 | 1.00 |
| PWY | biosynthesis I | | | | | | | | | |
| | (via L- | | | | | | | | | |
| | ornithine) | | | | | | | | | |
| VALSYN- | L-valine | 44 | 0.006304 | 2.86 | 41221.30 | 24359.88 | 1.00 | 14398.84 | 11832.67 | 1.00 |
| PWY | biosynthesis | | | | | | | | | |
| ANAGLYCOLYSIS- | glycolysis III | 45 | 0.006284 | 2.57 | 39616.54 | 19547.45 | 1.00 | 15433.71 | 11898.53 | 1.00 |
| PWY | (from glucose) | | | | | | | | | |
| PWY0- | polymyxin | 46 | 0.006129 | 0.12 | 208.68 | 708.05 | 0.88 | 1669.87 | 3136.81 | 0.94 |
| 1338 | resistance | | | | | | | | | |
| PWY-6263 | superpathway | 47 | 0.005997 | 5.48 | 2196.54 | 3113.85 | 0.95 | 401.07 | 993.92 | 0.76 |
| | of menaquinol- | | | | | | | | | |
| | 8 biosynthesis | | | | | | | | | |
| | II | | | | | | | | | |
| PWY-7371 | 1,4-dihydroxy- | 48 | 0.005991 | 4.89 | 1049.32 | 1966.55 | 0.95 | 214.45 | 753.96 | 0.76 |
| | 6-naphthoate | | | | | | | | | |
| | biosynthesis II | | | | | | | | | |
| PWY-5896 | superpathway | 49 | 0.005912 | 0.24 | 740.62 | 1757.63 | 0.96 | 3131.89 | 4208.09 | 0.98 |
| | of menaquinol- | | | | | | | | | |
| | 10 biosynthesis | | | | | | | | | |
| PWY-6277 | superpathway | 50 | 0.005811 | 2.65 | 35079.61 | 17750.87 | 1.00 | 13242.31 | 10142.08 | 1.00 |
| | of 5- | | | | | | | | | |
| | aminoimidazole | | | | | | | | | |
| | ribonucleotide | | | | | | | | | |
| | biosynthesis | | | | | | | | | |
| PWY-5845 | superpathway | 51 | 0.005765 | 0.24 | 745.67 | 1779.05 | 0.96 | 3131.89 | 4208.09 | 0.98 |
| | of menaquinol- | | | | | | | | | |
| | 9 biosynthesis | | | | | | | | | |
| PWY-6123 | inosine-5′- | 52 | 0.005721 | 2.66 | 33605.59 | 16812.37 | 1.00 | 12632.90 | 9679.61 | 1.00 |
| | phosphate | | | | | | | | | |
| | biosynthesis I | | | | | | | | | |
| ORNARGDEG- | superpathway | 53 | 0.005704 | 0.14 | 226.66 | 792.76 | 0.81 | 1590.90 | 2877.92 | 0.94 |
| PWY | of L-arginine | | | | | | | | | |
| | and L- | | | | | | | | | |
| | ornithine | | | | | | | | | |
| | degradation | | | | | | | | | |
| ALL- | superpathway | 54 | 0.005499 | 0.22 | 660.84 | 1761.20 | 0.88 | 3017.70 | 4247.08 | 0.95 |
| CHORISMATE- | of chorismate | | | | | | | | | |
| PWY | metabolism | | | | | | | | | |
| PWY-6387 | UDP-N- | 55 | 0.005411 | 2.64 | 32982.38 | 16688.93 | 1.00 | 12506.69 | 9467.25 | 1.00 |
| | acetylmuramoyl- | | | | | | | | | |
| | pentapeptide | | | | | | | | | |
| | biosynthesis I | | | | | | | | | |
| | (meso- | | | | | | | | | |
| | diaminopimelate | | | | | | | | | |
| | containing) | | | | | | | | | |
| ECASYN- | enterobacterial | 56 | 0.005236 | 0.15 | 287.81 | 929.31 | 0.84 | 1973.75 | 3351.22 | 0.94 |
| PWY | common | | | | | | | | | |
| | antigen | | | | | | | | | |
| | biosynthesis | | | | | | | | | |
| PWY4FS-7 | phosphatidyl- | 57 | 0.004955 | 2.73 | 33519.39 | 19371.51 | 1.00 | 12271.48 | 9713.41 | 1.00 |
| | glycerol | | | | | | | | | |
| | biosynthesis I | | | | | | | | | |
| | (plastidic) | | | | | | | | | |
| ILEUSYN- | L-isoleucine | 58 | 0.004852 | 2.86 | 41221.30 | 24359.88 | 1.00 | 14398.84 | 11832.67 | 1.00 |
| PWY | biosynthesis I | | | | | | | | | |
| | (from | | | | | | | | | |
| | threonine) | | | | | | | | | |
| PWY-5505 | L-glutamate | 59 | 0.004847 | 3.28 | 23972.87 | 16186.06 | 1.00 | 7310.25 | 7222.51 | 1.00 |
| | and L- | | | | | | | | | |
| | glutamine | | | | | | | | | |
| | biosynthesis | | | | | | | | | |
| ARGDEG- | superpathway | 60 | 0.004835 | 0.14 | 226.66 | 792.76 | 0.81 | 1590.90 | 2877.92 | 0.94 |
| PWY | of L-arginine, | | | | | | | | | |
| | putrescine, and | | | | | | | | | |
| | 4- | | | | | | | | | |
| | aminobutanoate | | | | | | | | | |
| | degradation | | | | | | | | | |
| P125-PWY | superpathway | 61 | 0.004809 | 0.32 | 231.37 | 328.15 | 1.00 | 733.15 | 1571.48 | 0.99 |
| | of (R,R)- | | | | | | | | | |
| | butanediol | | | | | | | | | |
| | biosynthesis | | | | | | | | | |
| GLYCOL- | superpathway | 62 | 0.004791 | 0.16 | 284.90 | 791.20 | 0.97 | 1763.59 | 2790.66 | 0.95 |
| GLYOXDEG- | of glycol | | | | | | | | | |
| PWY | metabolism | | | | | | | | | |
| | and | | | | | | | | | |
| | degradation | | | | | | | | | |
| PWY-6386 | UDP-N- | 63 | 0.004758 | 2.69 | 33067.64 | 16734.16 | 1.00 | 12310.17 | 9381.48 | 1.00 |
| | acetylmuramoyl- | | | | | | | | | |
| | pentapeptide | | | | | | | | | |
| | biosynthesis II | | | | | | | | | |
| | (lysine- | | | | | | | | | |
| | containing) | | | | | | | | | |
| ARGSYNBSUB- | L-arginine | 64 | 0.004717 | 3.13 | 27062.34 | 18615.53 | 1.00 | 8634.15 | 7616.24 | 1.00 |
| PWY | biosynthesis II | | | | | | | | | |
| | (acetyl cycle) | | | | | | | | | |
| ENTBACSYN- | enterobactin | 65 | 0.004667 | 0.18 | 496.80 | 1444.67 | 0.90 | 2705.67 | 3835.84 | 0.95 |
| PWY | biosynthesis | | | | | | | | | |
| PWY-6385 | peptidoglycan | 66 | 0.004633 | 2.67 | 32618.20 | 16544.58 | 1.00 | 12234.28 | 9272.55 | 1.00 |
| | biosynthesis III | | | | | | | | | |
| | (mycobacteria) | | | | | | | | | |
| THRESYN- | superpathway | 67 | 0.004283 | 2.69 | 34985.56 | 17229.65 | 1.00 | 12990.68 | 10283.94 | 1.00 |
| PWY | of L-threonine | | | | | | | | | |
| | biosynthesis | | | | | | | | | |
| 1CMET2- | N10-formyl- | 68 | 0.004256 | 2.78 | 32237.40 | 17679.11 | 1.00 | 11590.50 | 9125.00 | 1.00 |
| PWY | tetrahydrofolate | | | | | | | | | |
| | biosynthesis | | | | | | | | | |
| CHLOROPHYLL- | chlorophyllide | 69 | 0.004180 | 20.42 | 41.09 | 387.43 | 0.45 | 2.01 | 16.04 | 0.12 |
| SYN | a biosynthesis I | | | | | | | | | |
| | (aerobic, light- | | | | | | | | | |
| | dependent) | | | | | | | | | |
| PWY-6121 | 5- | 70 | 0.004175 | 2.65 | 36122.21 | 18777.40 | 1.00 | 13634.17 | 10455.30 | 1.00 |
| | aminoimidazole | | | | | | | | | |
| | ribonucleotide | | | | | | | | | |
| | biosynthesis I | | | | | | | | | |
| PEPTIDOGLYCANSYN- | peptidoglycan | 71 | 0.004073 | 2.65 | 32818.46 | 16611.34 | 1.00 | 12377.64 | 9359.46 | 1.00 |
| PWY | biosynthesis I | | | | | | | | | |
| | (meso- | | | | | | | | | |
| | diaminopimelate | | | | | | | | | |
| | containing) | | | | | | | | | |
| PWY-5840 | superpathway | 72 | 0.003908 | 0.47 | 1968.52 | 3031.91 | 0.99 | 4165.06 | 4482.90 | 1.00 |
| | of menaquinol- | | | | | | | | | |
| | 7 biosynthesis | | | | | | | | | |
| GLUCOSE1PMETAB- | glucose and | 73 | 0.003862 | 0.36 | 1220.90 | 2500.90 | 1.00 | 3377.19 | 4632.05 | 0.98 |
| PWY | glucose-1- | | | | | | | | | |
| | phosphate | | | | | | | | | |
| | degradation | | | | | | | | | |
| PWY-5898 | superpathway | 74 | 0.003817 | 0.44 | 1842.63 | 2934.13 | 0.99 | 4162.12 | 4654.22 | 1.00 |
| | of menaquinol- | | | | | | | | | |
| | 12 biosynthesis | | | | | | | | | |
| THREOCAT- | superpathway | 75 | 0.003685 | 0.15 | 222.56 | 878.03 | 0.63 | 1442.16 | 3317.05 | 0.92 |
| PWY | of L-threonine | | | | | | | | | |
| | metabolism | | | | | | | | | |
| PWY-6737 | starch | 76 | 0.003682 | 3.02 | 46433.64 | 24911.85 | 1.00 | 15361.05 | 13716.01 | 1.00 |
| | degradation V | | | | | | | | | |
| PWY-5198 | factor 420 | 77 | 0.003618 | 9.78 | 83.15 | 311.29 | 0.41 | 8.50 | 56.00 | 0.10 |
| | biosynthesis | | | | | | | | | |
| PWY-7286 | 7-(3-amino-3- | 78 | 0.003512 | 9.43 | 96.80 | 355.08 | 0.39 | 10.27 | 67.59 | 0.09 |
| | carboxypropyl)- | | | | | | | | | |
| | wyosine | | | | | | | | | |
| | biosynthesis | | | | | | | | | |
| GOLPDLCAT- | superpathway | 79 | 0.003491 | 0.15 | 202.18 | 432.68 | 0.94 | 1327.51 | 3027.64 | 0.99 |
| PWY | of glycerol | | | | | | | | | |
| | degradation to | | | | | | | | | |
| | 1,3- | | | | | | | | | |
| | propanediol | | | | | | | | | |
| TRNA- | tRNA charging | 80 | 0.003406 | 2.68 | 33409.36 | 17003.72 | 1.00 | 12475.49 | 9660.68 | 1.00 |
| CHARGING- | | | | | | | | | | |
| PWY | | | | | | | | | | |
| PWY-5531 | chlorophyllide | 81 | 0.003376 | 12.96 | 40.43 | 387.08 | 0.45 | 3.12 | 29.33 | 0.13 |
| | a biosynthesis | | | | | | | | | |
| | II (anaerobic) | | | | | | | | | |
| PWY-5837 | 1,4-dihydroxy- | 82 | 0.003372 | 0.35 | 1068.41 | 2492.81 | 0.99 | 3094.02 | 3998.36 | 1.00 |
| | 2-naphthoate | | | | | | | | | |
| | biosynthesis I | | | | | | | | | |
| PANTO- | phospho- | 83 | 0.003348 | 2.99 | 27427.71 | 17827.49 | 1.00 | 9188.30 | 7627.50 | 1.00 |
| PWY | pantothenate | | | | | | | | | |
| | biosynthesis I | | | | | | | | | |
| PWY-6071 | superpathway | 84 | 0.003346 | 0.16 | 89.88 | 427.71 | 0.55 | 568.88 | 1706.56 | 0.90 |
| | of | | | | | | | | | |
| | phenylethylamine | | | | | | | | | |
| | degradation | | | | | | | | | |
| PWY-6349 | CDP-archaeol | 85 | 0.003344 | 9.74 | 93.10 | 337.26 | 0.39 | 9.56 | 61.83 | 0.09 |
| | biosynthesis | | | | | | | | | |
| PWY-5899 | superpathway | 86 | 0.003325 | 0.44 | 1842.63 | 2934.13 | 0.99 | 4162.12 | 4654.22 | 1.00 |
| | of menaquinol- | | | | | | | | | |
| | 13 biosynthesis | | | | | | | | | |
| PWY-2942 | L-lysine | 87 | 0.00331 | 2.63 | 37924.77 | 18778.14 | 1.00 | 14401.68 | 11021.52 | 1.00 |
| | biosynthesis III | | | | | | | | | |
| PWY-5863 | superpathway | 88 | 0.003302 | 0.36 | 1152.27 | 2434.84 | 0.99 | 3219.88 | 3983.44 | 1.00 |
| | of phylloquinol | | | | | | | | | |
| | biosynthesis | | | | | | | | | |
| PWY-5088 | L-glutamate | 89 | 0.003221 | 6.96 | 209.24 | 1073.54 | 0.39 | 30.06 | 372.41 | 0.17 |
| | degradation | | | | | | | | | |
| | VIII (to | | | | | | | | | |
| | propanoate) | | | | | | | | | |
| GALACTARDEG- | D-galactarate | 90 | 0.003178 | 0.30 | 457.74 | 911.54 | 1.00 | 1522.96 | 2545.94 | 0.98 |
| PWY | degradation I | | | | | | | | | |
| PWY-6350 | archaetidylinositol | 91 | 0.003156 | 9.68 | 88.45 | 322.32 | 0.39 | 9.14 | 59.35 | 0.09 |
| | biosynthesis | | | | | | | | | |
| CALVIN- | Calvin- | 92 | 0.003140 | 2.64 | 41919.47 | 23350.92 | 1.00 | 15850.71 | 12125.61 | 1.00 |
| PWY | Benson- | | | | | | | | | |
| | Bassham cycle | | | | | | | | | |
| PWY-5861 | superpathway | 93 | 0.003113 | 0.42 | 1653.88 | 3067.35 | 0.99 | 3894.44 | 4510.05 | 0.99 |
| | of demethyl- | | | | | | | | | |
| | menaquinol-8 | | | | | | | | | |
| | biosynthesis | | | | | | | | | |
| PWY4FS-8 | phosphatidyl- | 94 | 0.003073 | 2.73 | 33519.39 | 19371.51 | 1.00 | 12271.48 | 9713.41 | 1.00 |
| | glycerol | | | | | | | | | |
| | biosynthesis II | | | | | | | | | |
| | (non-plastidic) | | | | | | | | | |
| PWY-7159 | chlorophyllide | 95 | 0.003022 | 12.96 | 40.43 | 387.08 | 0.45 | 3.12 | 29.33 | 0.13 |
| | a biosynthesis | | | | | | | | | |
| | III (aerobic, | | | | | | | | | |
| | light | | | | | | | | | |
| | independent) | | | | | | | | | |
| PHOSLIPSYN- | superpathway | 96 | 0.002996 | 2.69 | 36158.35 | 20262.73 | 1.00 | 13420.46 | 10360.64 | 1.00 |
| PWY | of | | | | | | | | | |
| | phospholipid | | | | | | | | | |
| | biosynthesis I | | | | | | | | | |
| | (bacteria) | | | | | | | | | |
| PWY0- | 3- | 97 | 0.002930 | 0.12 | 82.25 | 397.30 | 0.61 | 688.95 | 2092.65 | 0.87 |
| 1277 | phenylpropanoate | | | | | | | | | |
| | and 3-(3- | | | | | | | | | |
| | hydroxyphenyl)pro- | | | | | | | | | |
| | panoate | | | | | | | | | |
| | degradation | | | | | | | | | |
| PWY-5304 | superpathway | 98 | 0.002906 | 3.96 | 8480.96 | 8403.25 | 0.99 | 2141.59 | 3792.29 | 0.99 |
| | of sulfur | | | | | | | | | |
| | oxidation | | | | | | | | | |
| | (Acidianus | | | | | | | | | |
| | ambivalens) | | | | | | | | | |
| PWY-6690 | cinnamate and | 99 | 0.002899 | 0.09 | 49.91 | 274.08 | 0.61 | 549.96 | 1846.32 | 0.87 |
| | 3- | | | | | | | | | |
| | hydroxycinnamate | | | | | | | | | |
| | degradation to | | | | | | | | | |
| | 2-oxopent-4- | | | | | | | | | |
| | enoate | | | | | | | | | |
| GLUCARGALACTSUPER- | superpathway | 100 | 0.002897 | 0.30 | 457.74 | 911.54 | 1.00 | 1522.96 | 2545.94 | 0.98 |
| PWY | of D-glucarate | | | | | | | | | |
| | and D- | | | | | | | | | |
| | galactarate | | | | | | | | | |
| | degradation | | | | | | | | | |

IBS = Irritable Bowel Syndrome; CDI = Clostridiodes difficile Infection; RFFR = Random Forest Feature Rank; # = Rank; MDA = Mean Decrease Accuracy; FC = Fold Change; M = Mean; SD = Standard Deviation; DP = Detection Prevalence;

TABLE 8
IBS vs Control delineation features - (metabolic pathway)

| | | | | FC | | | | | | |
|---|---|---|---|---|---|---|---|---|---|---|
| MetaCyc | | | | IBS | | | | | | |
| Pathway | Pathway | RFFR | | vs | Abundance (IBS) | | | Abundance (Ctrl) | | |
| ID | description | # | MDA | Ctrl | M | SD | DP | M | SD | DP |
| PWY-7431 | aromatic | 1 | 0.014123 | 14.22 | 887.36 | 3722.68 | 0.97 | 62.42 | 336.03 | 0.87 |
| | biogenic amine | | | | | | | | | |
| | degradation | | | | | | | | | |
| | (bacteria) | | | | | | | | | |
| PWY-5384 | sucrose | 2 | 0.011419 | 0.81 | 12053.55 | 10048.97 | 1.00 | 14862.20 | 23647.49 | 1.00 |
| | degradation IV | | | | | | | | | |
| | (sucrose | | | | | | | | | |
| | phosphorylase) | | | | | | | | | |
| PWY-7539 | 6- | 3 | 0.011042 | 1.18 | 28384.93 | 19568.26 | 1.00 | 24023.34 | 57775.64 | 1.00 |
| | hydroxymethyl- | | | | | | | | | |
| | dihydropterin | | | | | | | | | |
| | diphosphate | | | | | | | | | |
| | biosynthesis III | | | | | | | | | |
| | (Chlamydia) | | | | | | | | | |
| PWY-6147 | 6- | 4 | 0.010145 | 1.17 | 27827.44 | 19724.99 | 1.00 | 23773.45 | 55311.45 | 1.00 |
| | hydroxymethyl- | | | | | | | | | |
| | dihydropterin | | | | | | | | | |
| | diphosphate | | | | | | | | | |
| | biosynthesis I | | | | | | | | | |
| PWY-6897 | thiamin | 5 | 0.009322 | 1.15 | 29830.89 | 20011.30 | 1.00 | 25927.29 | 55804.92 | 1.00 |
| | salvage II | | | | | | | | | |
| FOLSYN- | superpathway | 6 | 0.009318 | 1.10 | 28875.19 | 16869.28 | 1.00 | 26199.43 | 57898.05 | 1.00 |
| PWY | of | | | | | | | | | |
| | tetrahydrofolate | | | | | | | | | |
| | biosynthesis | | | | | | | | | |
| | and salvage | | | | | | | | | |
| PWY-6612 | superpathway | 7 | 0.008412 | 1.10 | 26345.17 | 15621.53 | 1.00 | 23842.31 | 53794.55 | 1.00 |
| | of | | | | | | | | | |
| | tetrahydrofolate | | | | | | | | | |
| | biosynthesis | | | | | | | | | |
| PANTO- | phosphopantothenate | 8 | 0.008230 | 1.18 | 27427.71 | 17827.49 | 1.00 | 23307.92 | 51652.13 | 1.00 |
| PWY | biosynthesis I | | | | | | | | | |
| TRPSYN- | L-tryptophan | 9 | 0.008122 | 1.12 | 29664.21 | 16817.20 | 1.00 | 26384.56 | 58066.86 | 1.00 |
| PWY | biosynthesis | | | | | | | | | |
| PWY-6471 | peptidoglycan | 10 | 0.007584 | 0.76 | 11749.05 | 9689.53 | 1.00 | 15517.50 | 26202.50 | 1.00 |
| | biosynthesis | | | | | | | | | |
| | IV | | | | | | | | | |
| | (Enterococcus | | | | | | | | | |
| | faecium) | | | | | | | | | |
| AEROBACTINSYN- | aerobactin | 11 | 0.007503 | 1.44 | 33.89 | 199.40 | 0.34 | 23.54 | 124.70 | 0.55 |
| PWY | biosynthesis | | | | | | | | | |
| PWY-5862 | superpathway | 12 | 0.006770 | 0.42 | 580.37 | 1491.91 | 0.96 | 1368.48 | 7127.97 | 0.96 |
| | of | | | | | | | | | |
| | demethyl- | | | | | | | | | |
| | menaquinol-9 | | | | | | | | | |
| | biosynthesis | | | | | | | | | |
| RIBOSYN2- | flavin | 13 | 0.006589 | 1.11 | 28641.46 | 15772.99 | 1.00 | 25710.38 | 56666.93 | 1.00 |
| PWY | biosynthesis I | | | | | | | | | |
| | (bacteria and | | | | | | | | | |
| | plants) | | | | | | | | | |
| PWY-5850 | superpathway | 14 | 0.006559 | 0.43 | 740.62 | 1757.63 | 0.96 | 1715.12 | 8129.74 | 0.96 |
| | of menaquinol- | | | | | | | | | |
| | 6 biosynthesis | | | | | | | | | |
| | I | | | | | | | | | |
| PWY-5860 | superpathway | 15 | 0.006553 | 0.42 | 575.99 | 1472.32 | 0.96 | 1368.24 | 7127.97 | 0.96 |
| | of | | | | | | | | | |
| | demethylmena | | | | | | | | | |
| | quinol-6 | | | | | | | | | |
| | biosynthesis I | | | | | | | | | |
| THREOCAT- | superpathway | 16 | 0.006475 | 0.46 | 222.56 | 878.03 | 0.63 | 487.43 | 6491.20 | 0.77 |
| PWY | of L-threonine | | | | | | | | | |
| | metabolism | | | | | | | | | |
| PWY-5896 | superpathway | 17 | 0.006405 | 0.43 | 740.62 | 1757.63 | 0.96 | 1715.12 | 8129.74 | 0.96 |
| | of menaquinol- | | | | | | | | | |
| | 10 biosynthesis | | | | | | | | | |
| PWY-5973 | cis-vaccenate | 18 | 0.006371 | 1.06 | 39974.77 | 20024.39 | 1.00 | 37597.57 | 77633.61 | 1.00 |
| | biosynthesis | | | | | | | | | |
| PWY-7663 | gondoate | 19 | 0.006351 | 1.06 | 43224.23 | 21636.87 | 1.00 | 40750.28 | 88888.09 | 1.00 |
| | biosynthesis | | | | | | | | | |
| | (anaerobic) | | | | | | | | | |
| PWY-5845 | superpathway | 20 | 0.006241 | 0.43 | 745.67 | 1779.05 | 0.96 | 1715.46 | 8129.75 | 0.96 |
| | of menaquinol- | | | | | | | | | |
| | 9 biosynthesis | | | | | | | | | |
| PWY-6122 | 5- | 21 | 0.005919 | 1.03 | 35079.61 | 17750.87 | 1.00 | 34211.58 | 67927.32 | 1.00 |
| | aminoimidazole | | | | | | | | | |
| | ribonucleotide | | | | | | | | | |
| | biosynthesis II | | | | | | | | | |
| PWY-7159 | chlorophyllide | 22 | 0.005875 | 4.89 | 40.43 | 387.08 | 0.45 | 8.26 | 66.18 | 0.25 |
| | a biosynthesis | | | | | | | | | |
| | III (aerobic, | | | | | | | | | |
| | light | | | | | | | | | |
| | independent) | | | | | | | | | |
| PWY-5531 | chlorophyllide | 23 | 0.005705 | 4.89 | 40.43 | 387.08 | 0.45 | 8.26 | 66.18 | 0.25 |
| | a biosynthesis | | | | | | | | | |
| | II (anaerobic) | | | | | | | | | |
| PANTOSYN- | pantothenate | 24 | 0.005661 | 1.13 | 28076.23 | 16189.06 | 1.00 | 24898.21 | 53813.43 | 1.00 |
| PWY | and coenzyme | | | | | | | | | |
| | A biosynthesis | | | | | | | | | |
| | I | | | | | | | | | |
| PWY-6478 | GDP-D- | 25 | 0.005598 | 0.79 | 2886.94 | 2928.03 | 1.00 | 3648.36 | 12511.71 | 1.00 |
| | glycero- | | | | | | | | | |
| | &alpha;-D- | | | | | | | | | |
| | manno-heptose | | | | | | | | | |
| | biosynthesis | | | | | | | | | |
| PYRIDNUCSAL- | NAD salvage | 26 | 0.005370 | 1.07 | 29619.59 | 15707.18 | 1.00 | 27573.47 | 56793.93 | 1.00 |
| PWY | pathway I | | | | | | | | | |
| CHLOROPHYLL- | chlorophyllide | 27 | 0.005349 | 4.74 | 41.09 | 387.43 | 0.45 | 8.68 | 68.06 | 0.25 |
| SYN | a biosynthesis I | | | | | | | | | |
| | (aerobic, light- | | | | | | | | | |
| | dependent) | | | | | | | | | |
| P562-PWY | myo-inositol | 28 | 0.005304 | 4.89 | 7805.01 | 18297.70 | 1.00 | 1595.53 | 6010.83 | 1.00 |
| | degradation I | | | | | | | | | |
| NONMEVIPP- | methylerythritol | 29 | 0.005289 | 1.04 | 34272.25 | 17397.13 | 1.00 | 32817.44 | 66602.58 | 1.00 |
| PWY | phosphate | | | | | | | | | |
| | pathway I | | | | | | | | | |
| P221-PWY | octane | 30 | 0.005180 | 0.87 | 1918.80 | 2782.48 | 0.94 | 2201.27 | 4989.83 | 0.99 |
| | oxidation | | | | | | | | | |
| PWY-3781 | aerobic | 31 | 0.004797 | 14.00 | 18914.47 | 55652.73 | 0.97 | 1350.90 | 12245.02 | 0.85 |
| | respiration I | | | | | | | | | |
| | (cytochrome c) | | | | | | | | | |
| PWY-6703 | preQ0 | 32 | 0.004679 | 1.26 | 21142.14 | 17391.99 | 1.00 | 16750.81 | 43567.02 | 1.00 |
| | biosynthesis | | | | | | | | | |
| PYRIDNUCSYN- | NAD | 33 | 0.004640 | 1.08 | 28698.20 | 15283.44 | 1.00 | 26519.01 | 54429.15 | 1.00 |
| PWY | biosynthesis I | | | | | | | | | |
| | (from | | | | | | | | | |
| | aspartate) | | | | | | | | | |
| PWY-6588 | pyruvate | 34 | 0.004599 | 1.12 | 4021.28 | 4225.75 | 1.00 | 3584.20 | 7857.69 | 1.00 |
| | fermentation to | | | | | | | | | |
| | acetone | | | | | | | | | |
| THRESYN- | superpathway | 35 | 0.004566 | 1.03 | 34985.56 | 17229.65 | 1.00 | 33804.17 | 69499.08 | 1.00 |
| PWY | of L-threonine | | | | | | | | | |
| | biosynthesis | | | | | | | | | |
| PWY-6123 | inosine-5′- | 36 | 0.004536 | 1.03 | 33605.59 | 16812.37 | 1.00 | 32731.91 | 67616.56 | 1.00 |
| | phosphate | | | | | | | | | |
| | biosynthesis I | | | | | | | | | |
| COA-PWY | coenzyme A | 37 | 0.004452 | 1.03 | 31573.49 | 15899.61 | 1.00 | 30734.70 | 62246.18 | 1.00 |
| | biosynthesis I | | | | | | | | | |
| PWY-3001 | superpathway | 38 | 0.004356 | 1.05 | 36808.04 | 19082.06 | 1.00 | 34993.92 | 70100.63 | 1.00 |
| | of L-isoleucine | | | | | | | | | |
| | biosynthesis I | | | | | | | | | |
| PWY-6277 | superpathway | 39 | 0.004286 | 1.03 | 35079.61 | 17750.87 | 1.00 | 34211.58 | 67927.32 | 1.00 |
| | of 5- | | | | | | | | | |
| | aminoimidazole | | | | | | | | | |
| | ribonucleotide | | | | | | | | | |
| | biosynthesis | | | | | | | | | |
| PWY-7377 | cob(II)yrinate | 40 | 0.004245 | 0.91 | 4178.37 | 4919.06 | 1.00 | 4593.83 | 12482.29 | 1.00 |
| | a,c-diamide | | | | | | | | | |
| | biosynthesis I | | | | | | | | | |
| | (early cobalt | | | | | | | | | |
| | insertion) | | | | | | | | | |
| PWY-6892 | thiazole | 41 | 0.004164 | 0.88 | 24004.43 | 15083.44 | 1.00 | 27367.72 | 60807.66 | 1.00 |
| | biosynthesis I | | | | | | | | | |
| | (E. coli) | | | | | | | | | |
| PWY-7209 | superpathway | 42 | 0.004036 | 0.56 | 8.24 | 37.62 | 0.22 | 14.68 | 77.78 | 0.28 |
| | of pyrimidine | | | | | | | | | |
| | ribonucleosides | | | | | | | | | |
| | degradation | | | | | | | | | |
| P122-PWY | heterolactic | 43 | 0.004004 | 0.56 | 1916.48 | 3035.80 | 1.00 | 3434.87 | 7997.81 | 1.00 |
| | fermentation | | | | | | | | | |
| PWY-5121 | superpathway | 44 | 0.003978 | 0.89 | 28115.14 | 15593.29 | 1.00 | 31735.49 | 63961.74 | 1.00 |
| | of | | | | | | | | | |
| | geranylgeranyl | | | | | | | | | |
| | diphosphate | | | | | | | | | |
| | biosynthesis II | | | | | | | | | |
| | (via MEP) | | | | | | | | | |
| PWY-5507 | adenosylcobalamin | 45 | 0.003896 | 0.85 | 75.98 | 227.22 | 0.63 | 89.34 | 312.06 | 0.40 |
| | biosynthesis I | | | | | | | | | |
| | (early cobalt | | | | | | | | | |
| | insertion) | | | | | | | | | |
| CODH- | reductive | 46 | 0.003864 | 0.88 | 752.09 | 1110.08 | 0.88 | 858.65 | 2009.75 | 0.95 |
| PWY | acetyl | | | | | | | | | |
| | coenzyme A | | | | | | | | | |
| | pathway | | | | | | | | | |
| ASPASN- | superpathway | 47 | 0.003805 | 0.87 | 24106.22 | 15009.33 | 1.00 | 27802.72 | 61241.17 | 1.00 |
| PWY | of L-aspartate | | | | | | | | | |
| | and L- | | | | | | | | | |
| | asparagine | | | | | | | | | |
| | biosynthesis | | | | | | | | | |
| PWY-5005 | biotin | 48 | 0.003798 | 0.96 | 1570.46 | 2924.05 | 1.00 | 1637.01 | 6066.53 | 0.99 |
| | biosynthesis II | | | | | | | | | |
| ANAGLYCOLYSIS- | glycolysis III | 49 | 0.003794 | 1.03 | 39616.54 | 19547.45 | 1.00 | 38524.25 | 76074.72 | 1.00 |
| PWY | (from glucose) | | | | | | | | | |
| PWY-7219 | adenosine | 50 | 0.003776 | 1.04 | 38393.50 | 19673.06 | 1.00 | 36934.72 | 73745.19 | 1.00 |
| | ribonucleotides | | | | | | | | | |
| | de novo | | | | | | | | | |
| | biosynthesis | | | | | | | | | |
| PWY-6470 | peptidoglycan | 51 | 0.003776 | 0.82 | 1073.61 | 1624.85 | 0.94 | 1308.86 | 3807.12 | 0.98 |
| | biosynthesis V | | | | | | | | | |
| | (&beta;-lactam | | | | | | | | | |
| | resistance) | | | | | | | | | |
| PWY0- | anhydromuropeptides | 52 | 0.003769 | 0.87 | 9061.13 | 7252.08 | 1.00 | 10433.27 | 26429.41 | 1.00 |
| 1261 | recycling | | | | | | | | | |
| PWY-6387 | UDP-N- | 53 | 0.003709 | 1.03 | 32982.38 | 16688.93 | 1.00 | 31963.32 | 63342.58 | 1.00 |
| | acetylmuramoyl- | | | | | | | | | |
| | pentapeptide | | | | | | | | | |
| | biosynthesis I | | | | | | | | | |
| | (meso- | | | | | | | | | |
| | diaminopimelate | | | | | | | | | |
| | containing) | | | | | | | | | |
| PWY-5097 | L-lysine | 54 | 0.003698 | 1.04 | 37580.67 | 18590.79 | 1.00 | 36065.98 | 71069.75 | 1.00 |
| | biosynthesis | | | | | | | | | |
| | VI | | | | | | | | | |
| PWY-7560 | methylerythritol | 55 | 0.003697 | 1.04 | 34272.25 | 17397.13 | 1.00 | 32817.44 | 66602.58 | 1.00 |
| | phosphate | | | | | | | | | |
| | pathway II | | | | | | | | | |
| 1CMET2- | N10-formyl- | 56 | 0.003683 | 1.09 | 32237.40 | 17679.11 | 1.00 | 29667.15 | 61241.36 | 1.00 |
| PWY | tetrahydrofolate | | | | | | | | | |
| | biosynthesis | | | | | | | | | |
| PEPTIDOGLYCANSYN- | peptidoglycan | 57 | 0.003643 | 1.03 | 32818.46 | 16611.34 | 1.00 | 31783.86 | 62985.20 | 1.00 |
| PWY | biosynthesis I | | | | | | | | | |
| | (meso- | | | | | | | | | |
| | diaminopimelate | | | | | | | | | |
| | containing) | | | | | | | | | |
| PWY-5971 | palmitate | 58 | 0.003616 | 0.86 | 11036.11 | 9374.84 | 0.97 | 12902.75 | 46991.68 | 1.00 |
| | biosynthesis II | | | | | | | | | |
| | (bacteria and | | | | | | | | | |
| | plants) | | | | | | | | | |
| P124-PWY | Bifidobacterium | 59 | 0.003573 | 0.49 | 2397.16 | 3856.49 | 1.00 | 4916.18 | 11478.79 | 1.00 |
| | shunt | | | | | | | | | |
| HCAMHPDEG- | 3- | 60 | 0.003547 | 0.22 | 49.91 | 274.08 | 0.61 | 224.09 | 3798.41 | 0.69 |
| PWY | phenylpropanoate | | | | | | | | | |
| | and 3-(3- | | | | | | | | | |
| | hydroxyphenyl)pro- | | | | | | | | | |
| | panoate | | | | | | | | | |
| | degradation to | | | | | | | | | |
| | 2-oxopent-4- | | | | | | | | | |
| | enoate | | | | | | | | | |
| GLYCOCAT- | glycogen | 61 | 0.003545 | 0.92 | 36577.81 | 20297.09 | 1.00 | 39548.53 | 74794.98 | 1.00 |
| PWY | degradation I | | | | | | | | | |
| | (bacterial) | | | | | | | | | |
| PWY-7332 | superpathway | 62 | 0.003526 | 1.64 | 1866.86 | 3130.64 | 0.99 | 1135.16 | 3807.64 | 0.99 |
| | of UDP-N- | | | | | | | | | |
| | acetylglucosamine- | | | | | | | | | |
| | derived O- | | | | | | | | | |
| | antigen | | | | | | | | | |
| | building blocks | | | | | | | | | |
| | biosynthesis | | | | | | | | | |
| PWY0-321 | phenylacetate | 63 | 0.003478 | 0.25 | 97.88 | 458.92 | 0.59 | 384.08 | 5893.00 | 0.73 |
| | degradation I | | | | | | | | | |
| | (aerobic) | | | | | | | | | |
| CALVIN- | Calvin- | 64 | 0.003453 | 1.08 | 41919.47 | 23350.92 | 1.00 | 38789.56 | 77297.80 | 1.00 |
| PWY | Benson- | | | | | | | | | |
| | Bassham cycle | | | | | | | | | |
| PWY-6891 | thiazole | 65 | 0.003426 | 0.69 | 4147.43 | 4624.29 | 1.00 | 5971.37 | 17637.92 | 1.00 |
| | biosynthesis II | | | | | | | | | |
| | (Bacillus) | | | | | | | | | |
| TYRFUMCAT- | L-tyrosine | 66 | 0.003394 | 162.96 | 8292.04 | 25541.88 | 0.71 | 50.88 | 672.98 | 0.49 |
| PWY | degradation I | | | | | | | | | |
| PWY-6901 | superpathway | 67 | 0.003392 | 0.88 | 14133.50 | 11721.70 | 1.00 | 16138.44 | 42305.62 | 1.00 |
| | of glucose and | | | | | | | | | |
| | xylose | | | | | | | | | |
| | degradation | | | | | | | | | |
| PWY-6121 | 5- | 68 | 0.003379 | 1.04 | 36122.21 | 18777.40 | 1.00 | 34599.61 | 69821.14 | 1.00 |
| | aminoimidazole | | | | | | | | | |
| | ribonucleotide | | | | | | | | | |
| | biosynthesis I | | | | | | | | | |
| PWY-6386 | UDP-N- | 69 | 0.003369 | 1.03 | 33067.64 | 16734.16 | 1.00 | 32031.44 | 63604.98 | 1.00 |
| | acetylmuramoyl- | | | | | | | | | |
| | pentapeptide | | | | | | | | | |
| | biosynthesis II | | | | | | | | | |
| | (lysine- | | | | | | | | | |
| | containing) | | | | | | | | | |
| PWY-6385 | peptidoglycan | 70 | 0.003368 | 1.04 | 32618.20 | 16544.58 | 1.00 | 31498.67 | 62616.82 | 1.00 |
| | biosynthesis III | | | | | | | | | |
| | (mycobacteria) | | | | | | | | | |
| PWY-5104 | L-isoleucine | 71 | 0.003362 | 1.06 | 40466.33 | 21816.16 | 1.00 | 38032.50 | 72813.36 | 1.00 |
| | biosynthesis | | | | | | | | | |
| | IV | | | | | | | | | |
| PWY-6071 | superpathway | 72 | 0.00336 | 0.24 | 89.88 | 427.71 | 0.55 | 374.41 | 5866.29 | 0.72 |
| | of | | | | | | | | | |
| | phenylethylamine | | | | | | | | | |
| | degradation | | | | | | | | | |
| RHAMCAT- | L-rhamnose | 73 | 0.003357 | 0.92 | 12302.08 | 9773.21 | 1.00 | 13374.68 | 37212.50 | 1.00 |
| PWY | degradation I | | | | | | | | | |
| PWY-7229 | superpathway | 74 | 0.003348 | 1.05 | 39152.22 | 20656.79 | 1.00 | 37269.82 | 76809.15 | 1.00 |
| | of adenosine | | | | | | | | | |
| | nucleotides de | | | | | | | | | |
| | novo | | | | | | | | | |
| | biosynthesis I | | | | | | | | | |
| PWY-5180 | toluene | 75 | 0.003289 | 1.15 | 560.43 | 1701.69 | 0.72 | 487.77 | 5058.33 | 0.71 |
| | degradation I | | | | | | | | | |
| | (aerobic) (via | | | | | | | | | |
| | o-cresol) | | | | | | | | | |
| P461-PWY | hexitol | 76 | 0.003286 | 0.99 | 6458.37 | 8233.31 | 0.99 | 6535.35 | 17451.16 | 1.00 |
| | fermentation to | | | | | | | | | |
| | lactate, | | | | | | | | | |
| | formate, | | | | | | | | | |
| | ethanol and | | | | | | | | | |
| | acetate | | | | | | | | | |
| PWY-7221 | guanosine | 77 | 0.003258 | 1.02 | 34118.96 | 16764.40 | 1.00 | 33485.60 | 67311.36 | 1.00 |
| | ribonucleotides | | | | | | | | | |
| | de novo | | | | | | | | | |
| | biosynthesis | | | | | | | | | |
| GLUTORN- | L-ornithine | 78 | 0.003245 | 1.19 | 28029.94 | 21071.03 | 1.00 | 23616.52 | 46464.24 | 1.00 |
| PWY | biosynthesis | | | | | | | | | |
| PWY-1861 | formaldehyde | 79 | 0.003241 | 0.95 | 5230.18 | 6529.30 | 0.99 | 5534.53 | 15193.86 | 1.00 |
| | assimilation II | | | | | | | | | |
| | (RuMP Cycle) | | | | | | | | | |
| PWY-6609 | adenine and | 80 | 0.003216 | 0.86 | 27163.52 | 16702.31 | 1.00 | 31727.63 | 63046.81 | 1.00 |
| | adenosine | | | | | | | | | |
| | salvage III | | | | | | | | | |
| ANAEROFRUCAT- | homolactic | 81 | 0.003208 | 0.86 | 22408.10 | 13981.72 | 1.00 | 25969.53 | 52349.57 | 1.00 |
| PWY | fermentation | | | | | | | | | |
| P162-PWY | L-glutamate | 82 | 0.003202 | 1.54 | 691.93 | 1607.02 | 0.96 | 448.71 | 887.20 | 0.99 |
| | degradation V | | | | | | | | | |
| | (via | | | | | | | | | |
| | hydroxyglutarate) | | | | | | | | | |
| PWY-6396 | superpathway | 83 | 0.003194 | 1.01 | 275.96 | 417.27 | 1.00 | 273.69 | 669.20 | 0.99 |
| | of 2,3- | | | | | | | | | |
| | butanediol | | | | | | | | | |
| | biosynthesis | | | | | | | | | |
| P125-PWY | superpathway | 84 | 0.003160 | 1.01 | 231.37 | 328.15 | 1.00 | 229.19 | 592.66 | 0.99 |
| | of (R,R)- | | | | | | | | | |
| | butanediol | | | | | | | | | |
| | biosynthesis | | | | | | | | | |
| PWYG- | mycolate | 85 | 0.003135 | 0.89 | 16761.86 | 12981.23 | 1.00 | 18886.83 | 63656.02 | 1.00 |
| 321 | biosynthesis | | | | | | | | | |
| PWY-6163 | chorismate | 86 | 0.003131 | 1.07 | 36411.39 | 19257.17 | 1.00 | 34156.88 | 67511.88 | 1.00 |
| | biosynthesis | | | | | | | | | |
| | from 3- | | | | | | | | | |
| | dehydroquinate | | | | | | | | | |
| PWY-5686 | UMP | 87 | 0.003129 | 1.06 | 38378.86 | 20600.14 | 1.00 | 36137.39 | 71110.33 | 1.00 |
| | biosynthesis | | | | | | | | | |
| PWY-5705 | allantoin | 88 | 0.003119 | 0.99 | 834.17 | 801.60 | 1.00 | 846.36 | 1653.24 | 1.00 |
| | degradation to | | | | | | | | | |
| | glyoxylate III | | | | | | | | | |
| PWY0- | 3- | 89 | 0.003119 | 0.26 | 82.25 | 397.30 | 0.61 | 318.48 | 5027.86 | 0.69 |
| 1277 | phenylpropanoate | | | | | | | | | |
| | and 3-(3-hydroxy- | | | | | | | | | |
| | phenyl)propanoate | | | | | | | | | |
| | degradation | | | | | | | | | |
| GLUCUROCAT- | superpathway | 90 | 0.003117 | 0.85 | 9930.86 | 6803.57 | 1.00 | 11711.24 | 23892.96 | 1.00 |
| PWY | of &beta;-D- | | | | | | | | | |
| | glucuronide | | | | | | | | | |
| | and D- | | | | | | | | | |
| | glucuronate | | | | | | | | | |
| | degradation | | | | | | | | | |
| FASYN- | superpathway | 91 | 0.003117 | 0.88 | 13225.39 | 10552.14 | 1.00 | 15098.44 | 51390.08 | 1.00 |
| INITIAL- | of fatty acid | | | | | | | | | |
| PWY | biosynthesis | | | | | | | | | |
| | initiation (E. | | | | | | | | | |
| | coli) | | | | | | | | | |
| ARGORNPROST- | arginine, | 92 | 0.003117 | 0.61 | 1410.86 | 2008.22 | 1.00 | 2313.51 | 6466.68 | 1.00 |
| PWY | ornithine and | | | | | | | | | |
| | proline | | | | | | | | | |
| | interconversion | | | | | | | | | |
| PWY-5103 | L-isoleucine | 93 | 0.003077 | 1.08 | 38191.84 | 21780.55 | 1.00 | 35363.64 | 68670.88 | 1.00 |
| | biosynthesis III | | | | | | | | | |
| PWY-5667 | CDP- | 94 | 0.003073 | 1.08 | 39618.43 | 22110.97 | 1.00 | 36676.06 | 68483.69 | 1.00 |
| | diacylglycerol | | | | | | | | | |
| | biosynthesis I | | | | | | | | | |
| PWY0-862 | (5Z)-dodec-5- | 95 | 0.003031 | 0.88 | 15346.68 | 11982.52 | 1.00 | 17509.60 | 58833.48 | 1.00 |
| | enoate | | | | | | | | | |
| | biosynthesis | | | | | | | | | |
| PWY-6690 | cinnamate and | 96 | 0.003024 | 0.22 | 49.91 | 274.08 | 0.61 | 224.09 | 3798.41 | 0.69 |
| | 3- | | | | | | | | | |
| | hydroxycinnamate | | | | | | | | | |
| | degradation to | | | | | | | | | |
| | 2-oxopent-4- | | | | | | | | | |
| | enoate | | | | | | | | | |
| PWY-5088 | L-glutamate | 97 | 0.003003 | 3.17 | 209.24 | 1073.54 | 0.39 | 66.06 | 357.04 | 0.42 |
| | degradation | | | | | | | | | |
| | VIII (to | | | | | | | | | |
| | propanoate) | | | | | | | | | |
| PWY0- | CDP- | 98 | 0.002978 | 1.08 | 39618.43 | 22110.97 | 1.00 | 36676.06 | 68483.69 | 1.00 |
| 1319 | diacylglycerol | | | | | | | | | |
| | biosynthesis II | | | | | | | | | |
| LACTOSECAT- | lactose and | 99 | 0.002966 | 0.85 | 476.71 | 937.69 | 0.95 | 559.72 | 2143.31 | 0.99 |
| PWY | galactose | | | | | | | | | |
| | degradation I | | | | | | | | | |
| PWY-2941 | L-lysine | 100 | 0.002951 | 0.67 | 2868.11 | 3966.27 | 1.00 | 4274.30 | 9871.39 | 1.00 |
| | biosynthesis II | | | | | | | | | |

IBS = Irritable Bowel Syndrome;
Ctrl = Control;
RFFR = Random Forest Feature Rank;
# = Rank;
MDA = Mean Decrease Accuracy;
FC = Fold Change;
M = Mean;
SD = Standard Deviation;
DP = Detection Prevalence;

TABLE 9
IBS vs Control delineation features - (taxonomy)

| Feature | | | FC | | | | | | |
|---|---|---|---|---|---|---|---|---|---|
| (Taxonomic order: Kingdom; | | | IBS | Abundance | | | Abundance | | |
| Phylum; Class; Order; | RFFR | | vs | (Ctrl) | | | (IBS) | | |
| Family; Genus; Species) | # | MDA | Ctrl | M | SD | DP | M | SD | DP |
| Bacteria; Other; Other; Other; | 1 | 0.018394 | 1.007746 | 0.005479 | 0.021341 | 0.819038 | 0.005521 | 0.011388 | 0.966102 |
| Other; Other; Other | | | | | | | | | |
| Bacteria; Firmicutes; Clostridia; | 2 | 0.012333 | 3.716663 | 0.002311 | 0.004024 | 0.911088 | 0.008589 | 0.017120 | 0.903148 |
| Clostridiales; Lachnospiraceae; | | | | | | | | | |
| Anaerostipes; Other | | | | | | | | | |
| Bacteria; Firmicutes; Other; | 3 | 0.012236 | 1.476355 | 0.000772 | 0.001531 | 0.731172 | 0.001140 | 0.001970 | 0.869249 |
| Other; Other; Other; Other | | | | | | | | | |
| Bacteria; Bacteroidetes; | 4 | 0.010558 | 44.235186 | 0.000000 | 0.000004 | 0.006276 | 0.000009 | 0.000033 | 0.121065 |
| Sphingobacteriia; Sphingobacteriales; | | | | | | | | | |
| Sphingobacteriaceae; Pedobacter; | | | | | | | | | |
| Other | | | | | | | | | |
| Bacteria; Firmicutes; Clostridia; | 5 | 0.010242 | 2.249361 | 0.013600 | 0.022700 | 0.991632 | 0.030591 | 0.053003 | 0.924939 |
| Clostridiales; Lachnospiraceae; | | | | | | | | | |
| Blautia; Other | | | | | | | | | |
| Bacteria; Firmicutes; Bacilli; | 6 | 0.010107 | 3.008967 | 0.000579 | 0.011930 | 0.434100 | 0.001743 | 0.006303 | 0.602906 |
| Lactobacillales; Streptococcaceae; | | | | | | | | | |
| Streptococcus; Streptococcus | | | | | | | | | |
| thermophilus | | | | | | | | | |
| Bacteria; Firmicutes; Bacilli; | 7 | 0.009640 | 1.648123 | 0.000149 | 0.000878 | 0.277197 | 0.000246 | 0.000996 | 0.559322 |
| Lactobacillales; Streptococcaceae; | | | | | | | | | |
| Streptococcus; Streptococcus | | | | | | | | | |
| parasanguinis | | | | | | | | | |
| Bacteria; Actinobacteria; Actinobacteria; | 8 | 0.009473 | 0.499128 | 0.003260 | 0.011115 | 0.519874 | 0.001627 | 0.007075 | 0.292978 |
| Coriobacteriales; Coriobacteriaceae; | | | | | | | | | |
| Collinsella; Other | | | | | | | | | |
| Bacteria; Firmicutes; Clostridia; | 9 | 0.009386 | 0.749057 | 0.041475 | 0.042224 | 0.967573 | 0.031067 | 0.038784 | 0.978208 |
| Clostridiales; Ruminococcaceae; | | | | | | | | | |
| Faecalibacterium; Other | | | | | | | | | |
| Bacteria; Actinobacteria; Coriobacteriia; | 10 | 0.009017 | 0.129289 | 0.010594 | 0.025323 | 0.555439 | 0.001370 | 0.005518 | 0.351090 |
| Coriobacteriales; Coriobacteriaceae; | | | | | | | | | |
| Collinsella; Collinsella aerofaciens | | | | | | | | | |
| Bacteria; Candidatus | 11 | 0.008915 | 14.347685 | 0.000016 | 0.000094 | 0.109833 | 0.000232 | 0.001213 | 0.295400 |
| Saccharibacteria; Saccharibacteria_{—} | | | | | | | | | |
| genera_incertae_sedis; Other; | | | | | | | | | |
| Other; Other; Other | | | | | | | | | |
| Bacteria; Firmicutes; Clostridia; | 12 | 0.008694 | 3.868790 | 0.000389 | 0.001534 | 0.315900 | 0.001507 | 0.004129 | 0.435835 |
| Clostridiales; Lachnospiraceae; | | | | | | | | | |
| Blautia; Blautia stercoris | | | | | | | | | |
| Bacteria; Proteobacteria; | 13 | 0.008468 | 0.311194 | 0.000389 | 0.006951 | 0.187238 | 0.000121 | 0.000994 | 0.394673 |
| Gammaproteobacteria; Pseudomonadales; | | | | | | | | | |
| Pseudomonadaceae; Pseudomonas; | | | | | | | | | |
| Other | | | | | | | | | |
| Bacteria; Firmicutes; Clostridia; | 14 | 0.008360 | 10.002237 | 0.000189 | 0.001137 | 0.438285 | 0.001891 | 0.008662 | 0.590799 |
| Clostridiales; Peptostreptococcaceae; | | | | | | | | | |
| Intestinibacter; Other | | | | | | | | | |
| Bacteria; Firmicutes; Clostridia; | 15 | 0.008041 | 4.587045 | 0.000891 | 0.003813 | 0.756276 | 0.004088 | 0.010861 | 0.801453 |
| Clostridiales; Peptostreptococcaceae; | | | | | | | | | |
| Other; Other | | | | | | | | | |
| Bacteria; Bacteroidetes; Bacteroidia; | 16 | 0.007743 | 0.513588 | 0.041765 | 0.060175 | 0.972803 | 0.021450 | 0.045829 | 0.927361 |
| Bacteroidales; Bacteroidaceae; | | | | | | | | | |
| Bacteroides; Bacteroides | | | | | | | | | |
| vulgatus | | | | | | | | | |
| Bacteria; Bacteroidetes; Bacteroidia; | 17 | 0.007300 | 1.615243 | 0.010596 | 0.035655 | 0.311715 | 0.017116 | 0.050016 | 0.539952 |
| Bacteroidales; Bacteroidaceae; | | | | | | | | | |
| Bacteroides; Bacteroides | | | | | | | | | |
| plebeius | | | | | | | | | |
| Bacteria; Firmicutes; Clostridia; | 18 | 0.006978 | 2.262828 | 0.002631 | 0.004823 | 0.891213 | 0.005954 | 0.013082 | 0.893462 |
| Clostridiales; Lachnospiraceae; | | | | | | | | | |
| Roseburia; Other | | | | | | | | | |
| Bacteria; Firmicutes; Bacilli; | 19 | 0.006728 | 1.498175 | 0.000033 | 0.000095 | 0.231172 | 0.000049 | 0.000115 | 0.421308 |
| Lactobacillales; Carnobacteriaceae; | | | | | | | | | |
| Granulicatella; Other | | | | | | | | | |
| Bacteria; Firmicutes; Clostridia; | 20 | 0.006484 | 1.176775 | 0.016905 | 0.025464 | 0.755230 | 0.019894 | 0.026907 | 0.869249 |
| Clostridiales; Ruminococcaceae; | | | | | | | | | |
| Faecalibacterium; Faecalibacterium | | | | | | | | | |
| prausnitzii | | | | | | | | | |
| Bacteria; Bacteroidetes; Bacteroidia; | 21 | 0.006241 | 1.501339 | 0.026934 | 0.054373 | 0.777197 | 0.040437 | 0.064921 | 0.842615 |
| Bacteroidales; Bacteroidaceae; | | | | | | | | | |
| Bacteroides; Bacteroides dorei | | | | | | | | | |
| Bacteria; Actinobacteria; | 22 | 0.006237 | 0.142245 | 0.000809 | 0.004296 | 0.456067 | 0.000115 | 0.000526 | 0.389831 |
| Coriobacteriia; Eggerthellales; | | | | | | | | | |
| Eggerthellaceae; Adlercreutzia; | | | | | | | | | |
| Adlercreutzia equolifaciens | | | | | | | | | |
| Bacteria; Bacteroidetes; Bacteroidia; | 23 | 0.006185 | 1.108835 | 0.036984 | 0.104763 | 0.619247 | 0.041010 | 0.107593 | 0.777240 |
| Bacteroidales; Prevotellaceae; | | | | | | | | | |
| Prevotella; Other | | | | | | | | | |
| Bacteria; Firmicutes; Clostridia; | 24 | 0.006130 | 0.963933 | 0.047176 | 0.039074 | 0.991632 | 0.045474 | 0.037841 | 0.968523 |
| Clostridiales; Lachnospiraceae; | | | | | | | | | |
| Other; Other | | | | | | | | | |
| Bacteria; Firmicutes; Bacilli; | 25 | 0.005797 | 13.117334 | 0.000002 | 0.000022 | 0.029289 | 0.000024 | 0.000095 | 0.162228 |
| Bacillales; Bacillales_Incertae Sedis | | | | | | | | | |
| XI; Gemella; Other | | | | | | | | | |
| Bacteria; Firmicutes; Negativicutes; | 26 | 0.005660 | NA - | 0.000000 | 0.000000 | 0.000000 | 0.000048 | 0.000333 | 0.096852 |
| Veillonellales; Veillonellaceae; | | | Div | | | | | | |
| Veillonella; Veillonella | | | by 0 | | | | | | |
| infantium | | | | | | | | | |
| Bacteria; Firmicutes; Clostridia; | 27 | 0.005564 | 1.469854 | 0.004115 | 0.005620 | 0.901674 | 0.006049 | 0.009841 | 0.796610 |
| Clostridiales; Lachnospiraceae; | | | | | | | | | |
| Dorea; Other | | | | | | | | | |
| Bacteria; Firmicutes; Clostridia; | 28 | 0.005365 | 1.009624 | 0.026222 | 0.040290 | 0.913180 | 0.026474 | 0.041908 | 0.820823 |
| Clostridiales; Lachnospiraceae; | | | | | | | | | |
| Blautia; Blautia wexlerae | | | | | | | | | |
| Bacteria; Firmicutes; Clostridia; | 29 | 0.005248 | 1.256043 | 0.017321 | 0.019449 | 0.981172 | 0.021757 | 0.027256 | 0.934625 |
| Clostridiales; Lachnospiraceae; | | | | | | | | | |
| Lachnospiracea_incertae_sedis; | | | | | | | | | |
| Other | | | | | | | | | |
| Bacteria; Firmicutes; Clostridia; | 30 | 0.004950 | 2.391660 | 0.001974 | 0.005469 | 0.876569 | 0.004722 | 0.010883 | 0.857143 |
| Clostridiales; Lachnospiraceae; | | | | | | | | | |
| Fusicatenibacter; Other | | | | | | | | | |
| Bacteria; Bacteroidetes; Bacteroidia; | 31 | 0.004935 | 1.268464 | 0.030745 | 0.095100 | 0.539749 | 0.038999 | 0.113177 | 0.685230 |
| Bacteroidales; Prevotellaceae; | | | | | | | | | |
| Prevotella; Prevotella copri | | | | | | | | | |
| Bacteria; Cyanobacteria/Chloroplast; | 32 | 0.004899 | 5.476367 | 0.000125 | 0.001867 | 0.233264 | 0.000683 | 0.004707 | 0.382567 |
| Chloroplast; Chloroplast; | | | | | | | | | |
| Streptophyta; Other; Other | | | | | | | | | |
| Bacteria; Proteobacteria; | 33 | 0.004866 | 101182.303196 | 0.000000 | 0.000001 | 0.004184 | 0.005094 | 0.028774 | 0.099274 |
| Alphaproteobacteria; Caulobacterales; | | | | | | | | | |
| Caulobacteraceae; Caulobacter; | | | | | | | | | |
| Caulobacter segnis | | | | | | | | | |
| Bacteria; Proteobacteria; | 34 | 0.004862 | NA - | 0.000000 | 0.000000 | 0.000000 | 0.000009 | 0.000056 | 0.089588 |
| Betaproteobacteria; Nitrosomonadales; | | | Div | | | | | | |
| Methylophilaceae; Methylophilus; | | | by 0 | | | | | | |
| Other | | | | | | | | | |
| Bacteria; Firmicutes; Clostridia; | 35 | 0.004704 | 1.003530 | 0.016348 | 0.020690 | 0.988494 | 0.016406 | 0.016525 | 0.944310 |
| Clostridiales; Other; Other; Other | | | | | | | | | |
| Bacteria; Bacteroidetes; Bacteroidia; | 36 | 0.004631 | 0.650077 | 0.001006 | 0.004950 | 0.701883 | 0.000654 | 0.001669 | 0.748184 |
| Bacteroidales; Other; Other; Other | | | | | | | | | |
| Bacteria; Bacteroidetes; Bacteroidia; | 37 | 0.004626 | 0.562319 | 0.030320 | 0.042820 | 0.952929 | 0.017049 | 0.027156 | 0.903148 |
| Bacteroidales; Bacteroidaceae; | | | | | | | | | |
| Bacteroides; Bacteroides | | | | | | | | | |
| uniformis | | | | | | | | | |
| Bacteria; Firmicutes; Bacilli; | 38 | 0.004625 | 22.272015 | 0.000002 | 0.000023 | 0.050209 | 0.000044 | 0.000234 | 0.121065 |
| Lactobacillales; Streptococcaceae; | | | | | | | | | |
| Other; Other | | | | | | | | | |
| Bacteria; Firmicutes; Clostridia; | 39 | 0.004592 | 0.675049 | 0.011136 | 0.014246 | 0.933054 | 0.007517 | 0.010821 | 0.871671 |
| Clostridiales; Lachnospiraceae; | | | | | | | | | |
| Fusicatenibacter; | | | | | | | | | |
| Fusicatenibacter saccharivorans | | | | | | | | | |
| Bacteria; Firmicutes; Clostridia; | 40 | 0.004525 | 0.952312 | 0.000797 | 0.003787 | 0.583682 | 0.000759 | 0.002089 | 0.716707 |
| Clostridiales; Lachnospiraceae; | | | | | | | | | |
| Lachnoclostridium; [Clostridium] | | | | | | | | | |
| bolteae | | | | | | | | | |
| Bacteria; Proteobacteria; | 41 | 0.004522 | 14.064952 | 0.000002 | 0.000026 | 0.020921 | 0.000028 | 0.000177 | 0.133172 |
| Betaproteobacteria; Burkholderiales; | | | | | | | | | |
| Comamonadaceae; Other; Other | | | | | | | | | |
| Bacteria; Bacteroidetes; Bacteroidia; | 42 | 0.004481 | 0.839811 | 0.061864 | 0.066323 | 0.995816 | 0.051954 | 0.062313 | 0.961259 |
| Bacteroidales; Bacteroidaceae; | | | | | | | | | |
| Bacteroides; Other | | | | | | | | | |
| Bacteria; Firmicutes; Clostridia; | 43 | 0.004471 | 0.830226 | 0.018787 | 0.024996 | 0.982218 | 0.015597 | 0.018427 | 0.934625 |
| Clostridiales; Ruminococcaceae; | | | | | | | | | |
| Other; Other | | | | | | | | | |
| Bacteria; Firmicutes; Negativicutes; | 44 | 0.004449 | 3.265531 | 0.002311 | 0.007033 | 0.512552 | 0.007546 | 0.022229 | 0.631961 |
| Selenomonadales; Veillonellaceae; | | | | | | | | | |
| Dialister; Other | | | | | | | | | |
| Bacteria; Firmicutes; Clostridia; | 45 | 0.004449 | 0.151074 | 0.001034 | 0.005783 | 0.419456 | 0.000156 | 0.000820 | 0.36396 |
| Clostridiales; Lachnospiraceae; | | | | | | | | | |
| Blautia; Blautia hominis | | | | | | | | | |
| Bacteria; Proteobacteria; | 46 | 0.004420 | 123819.901979 | 0.000001 | 0.000015 | 0.003138 | 0.087316 | 0.269557 | 0.099274 |
| Betaproteobacteria; Burkholderiales; | | | | | | | | | |
| Burkholderiaceae; Burkholderia; | | | | | | | | | |
| Other | | | | | | | | | |
| Bacteria; Firmicutes; Clostridia; | 47 | 0.004342 | 2.825478 | 0.000261 | 0.001295 | 0.320084 | 0.000738 | 0.002749 | 0.450363 |
| Clostridiales; Peptostreptococcaceae; | | | | | | | | | |
| Romboutsia; Other | | | | | | | | | |
| Bacteria; Proteobacteria; | 48 | 0.004217 | NA - | 0.000000 | 0.000000 | 0.000000 | 0.000010 | 0.000036 | 0.096852 |
| Betaproteobacteria; Burkholderiales; | | | Div | | | | | | |
| Burkholderiaceae; Burkholderia; | | | by 0 | | | | | | |
| Burkholderia ambifaria | | | | | | | | | |
| Bacteria; Proteobacteria; | 49 | 0.004194 | 13.513031 | 0.000020 | 0.000091 | 0.140167 | 0.000274 | 0.000849 | 0.210654 |
| Betaproteobacteria; Other; Other; | | | | | | | | | |
| Other; Other | | | | | | | | | |
| Bacteria; Proteobacteria; | 50 | 0.004125 | NA - | 0.000000 | 0.000000 | 0.000000 | 0.000283 | 0.001381 | 0.101695 |
| Alphaproteobacteria; Caulobacterales; | | | Div | | | | | | |
| Caulobacteraceae; Caulobacter; | | | by 0 | | | | | | |
| Other | | | | | | | | | |
| Bacteria; Firmicutes; Clostridia; | 51 | 0.004081 | 0.000000 | 0.000334 | 0.001238 | 0.169456 | 0.000000 | 0.000000 | 0.000000 |
| Clostridiales; Lachnospiraceae; | | | | | | | | | |
| Dorea; Dorea formicigenerans | | | | | | | | | |
| Bacteria; Proteobacteria; | 52 | 0.004054 | 0.308358 | 0.018587 | 0.056891 | 0.816946 | 0.005732 | 0.028681 | 0.765133 |
| Gammaproteobacteria; Enterobacterales; | | | | | | | | | |
| Enterobacteriaceae; Other; Other | | | | | | | | | |
| Bacteria; Bacteroidetes; Bacteroidia; | 53 | 0.003999 | 1.495197 | 0.007223 | 0.026249 | 0.260460 | 0.010799 | 0.032470 | 0.433414 |
| Bacteroidales; Bacteroidaceae; | | | | | | | | | |
| Bacteroides; Bacteroides | | | | | | | | | |
| coprocola | | | | | | | | | |
| Bacteria; Proteobacteria; | 54 | 0.003960 | 386.174767 | 0.000000 | 0.000005 | 0.008368 | 0.000118 | 0.001758 | 0.099274 |
| Alphaproteobacteria; Rhizobiales; | | | | | | | | | |
| Phyllobacteriaceae; Phyllobacterium; | | | | | | | | | |
| Other | | | | | | | | | |
| Bacteria; Actinobacteria; | 55 | 0.003950 | 0.328857 | 0.000223 | 0.000872 | 0.456067 | 0.000073 | 0.000244 | 0.331719 |
| Actinobacteria; Coriobacteriales; | | | | | | | | | |
| Coriobacteriaceae; Other; Other | | | | | | | | | |
| Bacteria; Firmicutes; Clostridia; | 56 | 0.003944 | 0.670233 | 0.007882 | 0.013361 | 0.888075 | 0.005283 | 0.010157 | 0.767554 |
| Clostridiales; Lachnospiraceae; | | | | | | | | | |
| Blautia; Blautia obeum | | | | | | | | | |
| Bacteria; Firmicutes; Clostridia; | 57 | 0.003905 | 0.566696 | 0.007247 | 0.013333 | 0.910042 | 0.004107 | 0.005814 | 0.779661 |
| Clostridiales; Lachnospiraceae; | | | | | | | | | |
| Blautia; Blautia faecis | | | | | | | | | |
| Bacteria; Proteobacteria; | 58 | 0.003889 | NA - | 0.000000 | 0.000000 | 0.000000 | 0.000554 | 0.001748 | 0.096852 |
| Betaproteobacteria; Burkholderiales; | | | Div | | | | | | |
| Burkholderiaceae; Other; Other | | | by 0 | | | | | | |
| Bacteria; Actinobacteria; | 59 | 0.003789 | 0.339497 | 0.000169 | 0.000438 | 0.498954 | 0.000057 | 0.000132 | 0.440678 |
| Actinobacteria; Actinomycetales; | | | | | | | | | |
| Actinomycetaceae; Schaalia; Schaalia | | | | | | | | | |
| odontolytica | | | | | | | | | |
| Bacteria; Firmicutes; Clostridia; | 60 | 0.003757 | 0.525861 | 0.015276 | 0.024437 | 0.915272 | 0.008033 | 0.011088 | 0.849879 |
| Clostridiales; Lachnospiraceae; | | | | | | | | | |
| Blautia; Blautia luti | | | | | | | | | |
| Bacteria; Proteobacteria; | 61 | 0.003749 | 8.446078 | 0.000005 | 0.000044 | 0.024059 | 0.000039 | 0.000211 | 0.118644 |
| Alphaproteobacteria; Sphingomonadales; | | | | | | | | | |
| Sphingomonadaceae; Sphingomonas; | | | | | | | | | |
| Other | | | | | | | | | |
| Bacteria; Bacteroidetes; Bacteroidia; | 62 | 0.003732 | 0.909853 | 0.000241 | 0.001067 | 0.518828 | 0.000219 | 0.000524 | 0.639225 |
| Bacteroidales; Porphyromonadaceae; | | | | | | | | | |
| Odoribacter; Other | | | | | | | | | |
| Bacteria; Firmicutes; Clostridia; | 63 | 0.003685 | 0.979763 | 0.027343 | 0.033723 | 0.892259 | 0.026790 | 0.042600 | 0.927361 |
| Clostridiales; Lachnospiraceae; | | | | | | | | | |
| Not Available; [Eubacterium] | | | | | | | | | |
| rectale | | | | | | | | | |
| Bacteria; Firmicutes; Erysipelotrichia; | 64 | 0.003664 | 3.717831 | 0.000012 | 0.000046 | 0.188285 | 0.000046 | 0.000161 | 0.341404 |
| Erysipelotrichales; Erysipelotrichaceae; | | | | | | | | | |
| Solobacterium; Solobacterium | | | | | | | | | |
| moorei | | | | | | | | | |
| Bacteria; Firmicutes; Clostridia; | 65 | 0.003661 | 0.891194 | 0.007228 | 0.030628 | 0.802301 | 0.006442 | 0.014729 | 0.920097 |
| Clostridiales; Peptostreptococcaceae; | | | | | | | | | |
| Romboutsia; Romboutsia | | | | | | | | | |
| timonensis | | | | | | | | | |
| Bacteria; Bacteroidetes; Bacteroidia; | 66 | 0.003640 | 1.195185 | 0.001405 | 0.008557 | 0.092050 | 0.001679 | 0.006990 | 0.208232 |
| Bacteroidales; Bacteroidaceae; | | | | | | | | | |
| Bacteroides; Bacteroides | | | | | | | | | |
| coprophilus | | | | | | | | | |
| Bacteria; Firmicutes; Clostridia; | 67 | 0.003590 | 0.718857 | 0.006404 | 0.011919 | 0.855649 | 0.004604 | 0.008826 | 0.723971 |
| Clostridiales; Ruminococcaceae; | | | | | | | | | |
| Ruminococcus; Ruminococcus | | | | | | | | | |
| faecis | | | | | | | | | |
| Bacteria; Firmicutes; Clostridia; | 68 | 0.003561 | 1.364636 | 0.000871 | 0.003572 | 0.674686 | 0.001188 | 0.004845 | 0.714286 |
| Clostridiales; Lachnospiraceae; | | | | | | | | | |
| Roseburia; Roseburia hominis | | | | | | | | | |
| Bacteria; Firmicutes; Clostridia; | 69 | 0.003550 | 1.192065 | 0.016148 | 0.023196 | 0.965481 | 0.019249 | 0.028624 | 0.905569 |
| Clostridiales; Lachnospiraceae; | | | | | | | | | |
| Anaerostipes; Anaerostipes | | | | | | | | | |
| hadrus | | | | | | | | | |
| Bacteria; Bacteroidetes; Bacteroidia; | 70 | 0.003507 | 0.621597 | 0.002446 | 0.004682 | 0.764644 | 0.001521 | 0.002372 | 0.799031 |
| Bacteroidales; Odoribacteraceae; | | | | | | | | | |
| Odoribacter; Odoribacter | | | | | | | | | |
| splanchnicus | | | | | | | | | |
| Bacteria; Firmicutes; Clostridia; | 71 | 0.003468 | 1.506781 | 0.002996 | 0.007844 | 0.741632 | 0.004514 | 0.012317 | 0.876513 |
| Clostridiales; Peptostreptococcaceae; | | | | | | | | | |
| Intestinibacter; Intestinibacter | | | | | | | | | |
| bartlettii | | | | | | | | | |
| Bacteria; Firmicutes; Clostridia; | 72 | 0.003456 | 0.663227 | 0.004547 | 0.006250 | 0.803347 | 0.003015 | 0.004631 | 0.685230 |
| Clostridiales; Lachnospiraceae; | | | | | | | | | |
| Coprococcus; Coprococcus | | | | | | | | | |
| comes | | | | | | | | | |
| Bacteria; Firmicutes; Clostridia; | 73 | 0.003452 | 1.108955 | 0.000966 | 0.002406 | 0.737448 | 0.001071 | 0.002925 | 0.791768 |
| Clostridiales; Ruminococcaceae; | | | | | | | | | |
| Flavonifractor; Flavonifractor | | | | | | | | | |
| plautii | | | | | | | | | |
| Bacteria; Proteobacteria; | 74 | 0.003434 | 3.979368 | 0.000078 | 0.000419 | 0.219665 | 0.000310 | 0.000933 | 0.302663 |
| Betaproteobacteria; Burkholderiales; | | | | | | | | | |
| Other; Other; Other | | | | | | | | | |
| Bacteria; Actinobacteria; | 75 | 0.003422 | 0.541252 | 0.010770 | 0.034421 | 0.756276 | 0.005829 | 0.020871 | 0.799031 |
| Actinobacteria; Bifidobacteriales; | | | | | | | | | |
| Bifidobacteriaceae; Bifidobacterium; | | | | | | | | | |
| Other | | | | | | | | | |
| Bacteria; Firmicutes; Clostridia; | 76 | 0.003386 | 0.684883 | 0.009032 | 0.011433 | 0.851464 | 0.006186 | 0.008890 | 0.736077 |
| Clostridiales; Lachnospiraceae; | | | | | | | | | |
| Dorea; Dorea longicatena | | | | | | | | | |
| Bacteria; Firmicutes; Clostridia; | 77 | 0.003354 | 1.413307 | 0.003746 | 0.007142 | 0.809623 | 0.005294 | 0.008906 | 0.818402 |
| Clostridiales; Lachnospiraceae; | | | | | | | | | |
| Roseburia; Roseburia | | | | | | | | | |
| inulinivorans | | | | | | | | | |
| Bacteria; Firmicutes; Clostridia; | 78 | 0.003339 | 0.800991 | 0.002003 | 0.003530 | 0.778243 | 0.001605 | 0.002634 | 0.750605 |
| Clostridiales; Ruminococcaceae; | | | | | | | | | |
| Agathobaculum; Agathobaculum | | | | | | | | | |
| butyriciproducens | | | | | | | | | |
| Bacteria; Actinobacteria; | 79 | 0.003318 | 0.249526 | 0.000541 | 0.001754 | 0.474895 | 0.000135 | 0.000423 | 0.401937 |
| Coriobacteriia; Eggerthellales; | | | | | | | | | |
| Eggerthellaceae; Eggerthella; Eggerthella | | | | | | | | | |
| lenta | | | | | | | | | |
| Bacteria; Firmicutes; Clostridia; | 80 | 0.003248 | 4.372868 | 0.000058 | 0.000380 | 0.228033 | 0.000254 | 0.001565 | 0.326877 |
| Other; Other; Other; Other | | | | | | | | | |
| Bacteria; Bacteroidetes; Bacteroidia; | 81 | 0.003228 | 1.458281 | 0.001533 | 0.004041 | 0.702929 | 0.002235 | 0.007078 | 0.760291 |
| Bacteroidales; Bacteroidaceae; | | | | | | | | | |
| Bacteroides; Bacteroides | | | | | | | | | |
| ovatus | | | | | | | | | |
| Bacteria; Verrucomicrobia; | 82 | 0.003213 | 1.194589 | 0.008451 | 0.033406 | 0.643305 | 0.010095 | 0.050965 | 0.523002 |
| Verrucomicrobiae; Verrucomicrobiales; | | | | | | | | | |
| Verrucomicrobiaceae; Akkermansia; | | | | | | | | | |
| Other | | | | | | | | | |
| Bacteria; Bacteroidetes; Bacteroidia; | 83 | 0.003175 | 10.476223 | 0.000154 | 0.001694 | 0.024059 | 0.001615 | 0.015875 | 0.118644 |
| Bacteroidales; Prevotellaceae; | | | | | | | | | |
| Prevotellamassilia; Prevotellamassilia | | | | | | | | | |
| timonensis | | | | | | | | | |
| Bacteria; Firmicutes; Clostridia; | 84 | 0.003156 | 1.104880 | 0.001354 | 0.004548 | 0.874477 | 0.001496 | 0.016614 | 0.808717 |
| Clostridiales; Ruminococcaceae; | | | | | | | | | |
| Ruthenibacterium; Ruthenibacterium | | | | | | | | | |
| lactatiformans | | | | | | | | | |
| Bacteria; Firmicutes; Bacilli; | 85 | 0.003148 | 2.724265 | 0.000012 | 0.000040 | 0.216527 | 0.000034 | 0.000112 | 0.326877 |
| Lactobacillales; Streptococcaceae; | | | | | | | | | |
| Streptococcus; Streptococcus | | | | | | | | | |
| sanguinis | | | | | | | | | |
| Bacteria; Firmicutes; Clostridia; | 86 | 0.003145 | 1.605750 | 0.004544 | 0.010129 | 0.852510 | 0.007296 | 0.018218 | 0.820823 |
| Clostridiales; Lachnospiraceae; | | | | | | | | | |
| Roseburia; Roseburia intestinalis | | | | | | | | | |
| Bacteria; Firmicutes; Clostridia; | 87 | 0.003137 | 1.255456 | 0.000680 | 0.002129 | 0.750000 | 0.000853 | 0.002469 | 0.799031 |
| Clostridiales; Lachnospiraceae; | | | | | | | | | |
| Clostridium XIVa; Other | | | | | | | | | |
| Bacteria; Bacteroidetes; Bacteroidia; | 88 | 0.003125 | 0.660533 | 0.005622 | 0.013699 | 0.744770 | 0.003714 | 0.010739 | 0.673123 |
| Bacteroidales; Tannerellaceae; | | | | | | | | | |
| Parabacteroides; Parabacteroides | | | | | | | | | |
| distasonis | | | | | | | | | |
| Bacteria; Firmicutes; Clostridia; | 89 | 0.003125 | 1.444228 | 0.000260 | 0.000949 | 0.433054 | 0.000375 | 0.001684 | 0.503632 |
| Clostridiales; Ruminococcaceae; | | | | | | | | | |
| Gemmiger; Other | | | | | | | | | |
| Bacteria; Bacteroidetes; Other; Other; | 90 | 0.003110 | 1.917515 | 0.000343 | 0.002384 | 0.446653 | 0.000658 | 0.008037 | 0.554479 |
| Other; Other; Other | | | | | | | | | |
| Bacteria; Actinobacteria; Actinobacteria; | 91 | 0.003094 | 0.376749 | 0.012432 | 0.030734 | 0.647490 | 0.004684 | 0.011625 | 0.699758 |
| Bifidobacteriales; Bifidobacteriaceae; | | | | | | | | | |
| Bifidobacterium; Bifidobacterium | | | | | | | | | |
| adolescentis | | | | | | | | | |
| Bacteria; Firmicutes; Clostridia; | 92 | 0.003093 | 1.117535 | 0.002972 | 0.006360 | 0.816946 | 0.003321 | 0.006487 | 0.767554 |
| Clostridiales; Lachnospiraceae; | | | | | | | | | |
| Coprococcus; Other | | | | | | | | | |
| Bacteria; Bacteroidetes; Bacteroidia; | 93 | 0.003082 | 1.120969 | 0.005870 | 0.016013 | 0.892259 | 0.006580 | 0.020688 | 0.859564 |
| Bacteroidales; Rikenellaceae; | | | | | | | | | |
| Alistipes; Other | | | | | | | | | |
| Bacteria; Actinobacteria; Actinobacteria; | 94 | 0.003073 | 0.648229 | 0.001571 | 0.006672 | 0.605649 | 0.001018 | 0.003735 | 0.690073 |
| Bifidobacteriales; Bifidobacteriaceae; | | | | | | | | | |
| Bifidobacterium; Bifidobacterium | | | | | | | | | |
| longum | | | | | | | | | |
| Bacteria; Bacteroidetes; Bacteroidia; | 95 | 0.003063 | 1.220566 | 0.006204 | 0.016822 | 0.407950 | 0.007573 | 0.020157 | 0.556901 |
| Bacteroidales; Bacteroidaceae; | | | | | | | | | |
| Bacteroides; Bacteroides | | | | | | | | | |
| massiliensis | | | | | | | | | |
| Bacteria; Bacteroidetes; Bacteroidia; | 96 | 0.003052 | 1.169985 | 0.004785 | 0.007226 | 0.927824 | 0.005598 | 0.014935 | 0.898305 |
| Bacteroidales; Porphyromonadaceae; | | | | | | | | | |
| Parabacteroides; Other | | | | | | | | | |
| Bacteria; Firmicutes; Clostridia; | 97 | 0.003025 | 0.988898 | 0.000284 | 0.002487 | 0.049163 | 0.000280 | 0.002139 | 0.159806 |
| Clostridiales; Ruminococcaceae; | | | | | | | | | |
| Not Available; [Eubacterium] | | | | | | | | | |
| siraeum | | | | | | | | | |
| Bacteria; Firmicutes; Clostridia; | 98 | 0.002968 | 0.442577 | 0.001015 | 0.002715 | 0.754184 | 0.000449 | 0.001329 | 0.612591 |
| Clostridiales; Ruminococcaceae; | | | | | | | | | |
| Not Available; [Clostridium] | | | | | | | | | |
| leptum | | | | | | | | | |
| Bacteria; Firmicutes; Bacilli; | 99 | 0.002957 | 1.038942 | 0.007021 | 0.016525 | 0.938285 | 0.007294 | 0.018938 | 0.907990 |
| Lactobacillales; Streptococcaceae; | | | | | | | | | |
| Streptococcus; Other | | | | | | | | | |
| Bacteria; Firmicutes; Clostridia; | 100 | 0.002956 | 1.465496 | 0.000009 | 0.000050 | 0.106695 | 0.000013 | 0.000037 | 0.188862 |
| Clostridiales; Ruminococcaceae; | | | | | | | | | |
| Pseudoflavonifractor; Other | | | | | | | | | |

IBS = Irritable Bowel Syndrome;
Ctrl = Control;
RFFR = Random Forest Feature Rank;
# = Rank;
MDA = Mean Decrease Accuracy;
FC = Fold Change;
M = Mean;
SD = Standard Deviation;
DP = Detection Prevalence;

TABLE 10
IBD vs CDI delineation features - (taxonomy)

| Feature | | | | | | | | | |
|---|---|---|---|---|---|---|---|---|---|
| (Taxonomic order: Kingdom; | | | FC | Abundance | | | Abundance | | |
| Phylum; Class; Order; | RFFR | | IBS vs | (IBS) | | | (CDI) | | |
| Family; Genus; Species) | # | MDA | CDI | M | SD | DP | M | SD | DP |
| Bacteria; Firmicutes; Clostridia; | 1 | 0.033863 | 0.000233 | 0.000005 | 0.000037 | 0.026634 | 0.020265 | 0.056449 | 0.764317 |
| Clostridiales; Peptostreptococcaceae; | | | | | | | | | |
| Clostridioides; Clostridioides difficile | | | | | | | | | |
| Bacteria; Firmicutes; Clostridia; | 2 | 0.022370 | 9.117167 | 0.019249 | 0.028624 | 0.905569 | 0.002111 | 0.008309 | 0.275330 |
| Clostridiales; Lachnospiraceae; | | | | | | | | | |
| Anaerostipes; Anaerostipes hadrus | | | | | | | | | |
| Bacteria; Firmicutes; Clostridia; | 3 | 0.020724 | 14.516793 | 0.019894 | 0.026907 | 0.869249 | 0.001370 | 0.008726 | 0.204846 |
| Clostridiales; Ruminococcaceae; | | | | | | | | | |
| Faecalibacterium; Faecalibacterium | | | | | | | | | |
| prausnitzii | | | | | | | | | |
| Bacteria; Firmicutes; Clostridia; | 4 | 0.020152 | 27.958502 | 0.003321 | 0.006487 | 0.767554 | 0.000119 | 0.000698 | 0.110132 |
| Clostridiales; Lachnospiraceae; | | | | | | | | | |
| Coprococcus; Other | | | | | | | | | |
| Bacteria; Proteobacteria; | 5 | 0.018425 | 0.040278 | 0.005732 | 0.028681 | 0.765133 | 0.142299 | 0.194372 | 0.907489 |
| Gammaproteobacteria; Enterobacterales; | | | | | | | | | |
| Enterobacteriaceae; Other; Other | | | | | | | | | |
| Bacteria; Firmicutes; Clostridia; | 6 | 0.018083 | 11.321488 | 0.026790 | 0.042600 | 0.927361 | 0.002366 | 0.009054 | 0.438326 |
| Clostridiales; Lachnospiraceae; | | | | | | | | | |
| Not Available; [Eubacterium] | | | | | | | | | |
| rectale | | | | | | | | | |
| Bacteria; Firmicutes; Clostridia; | 7 | 0.017101 | 10.343451 | 0.005954 | 0.013082 | 0.893462 | 0.000576 | 0.002224 | 0.306167 |
| Clostridiales; Lachnospiraceae; | | | | | | | | | |
| Roseburia; Other | | | | | | | | | |
| Bacteria; Proteobacteria; | 8 | 0.016941 | 0.038377 | 0.001194 | 0.007142 | 0.610169 | 0.031113 | 0.060092 | 0.856828 |
| Gammaproteobacteria; Enterobacteriales; | | | | | | | | | |
| Enterobacteriaceae; Other; Other | | | | | | | | | |
| Bacteria; Firmicutes; Clostridia; | 9 | 0.016935 | 4.285574 | 0.001605 | 0.002634 | 0.750605 | 0.000374 | 0.002447 | 0.145374 |
| Clostridiales; Ruminococcaceae; | | | | | | | | | |
| Agathobaculum; Agathobaculum | | | | | | | | | |
| butyriciproducens | | | | | | | | | |
| Bacteria; Firmicutes; Clostridia; | 10 | 0.016799 | 6.882997 | 0.004722 | 0.010883 | 0.857143 | 0.000686 | 0.003778 | 0.220264 |
| Clostridiales; Lachnospiraceae; | | | | | | | | | |
| Fusicatenibacter; Other | | | | | | | | | |
| Bacteria; Firmicutes; Clostridia; | 11 | 0.016419 | 13.559941 | 0.006049 | 0.009841 | 0.796610 | 0.000446 | 0.002177 | 0.189427 |
| Clostridiales; Lachnospiraceae; | | | | | | | | | |
| Dorea; Other | | | | | | | | | |
| Bacteria; Firmicutes; Clostridia; | 12 | 0.016137 | 34.131598 | 0.013466 | 0.023548 | 0.791768 | 0.000395 | 0.002295 | 0.204846 |
| Clostridiales; Ruminococcaceae; | | | | | | | | | |
| Ruminococcus; Other | | | | | | | | | |
| Bacteria; Bacteroidetes; | 13 | 0.015514 | 0.759736 | 0.001521 | 0.002372 | 0.799031 | 0.002001 | 0.009758 | 0.178414 |
| Bacteroidia; Bacteroidales; | | | | | | | | | |
| Odoribacteraceae; Odoribacter; | | | | | | | | | |
| Odoribacter splanchnicus | | | | | | | | | |
| Bacteria; Firmicutes; Clostridia; | 14 | 0.015224 | 4.125927 | 0.016406 | 0.016525 | 0.944310 | 0.003976 | 0.012514 | 0.726872 |
| Clostridiales; Other; Other; Other | | | | | | | | | |
| Bacteria; Firmicutes; Clostridia; | 15 | 0.013148 | 3.278837 | 0.007517 | 0.010821 | 0.871671 | 0.002293 | 0.012148 | 0.312775 |
| Clostridiales; Lachnospiraceae; | | | | | | | | | |
| Fusicatenibacter; Fusicatenibacter | | | | | | | | | |
| saccharivorans | | | | | | | | | |
| Bacteria; Firmicutes; Clostridia; | 16 | 0.012788 | 6.601872 | 0.005294 | 0.008906 | 0.818402 | 0.000802 | 0.004536 | 0.198238 |
| Clostridiales; Lachnospiraceae; | | | | | | | | | |
| Roseburia; Roseburia inulinivorans | | | | | | | | | |
| Bacteria; Firmicutes; Clostridia; | 17 | 0.012671 | 8.124014 | 0.006186 | 0.008890 | 0.736077 | 0.000761 | 0.004308 | 0.162996 |
| Clostridiales; Lachnospiraceae; | | | | | | | | | |
| Dorea; Dorea longicatena | | | | | | | | | |
| Bacteria; Other; Other; Other; | 18 | 0.012176 | 8.581308 | 0.005521 | 0.011388 | 0.966102 | 0.000643 | 0.004047 | 0.594714 |
| Other; Other; Other | | | | | | | | | |
| Bacteria; Firmicutes; Bacilli; | 19 | 0.012051 | 0.010131 | 0.000496 | 0.004249 | 0.322034 | 0.048932 | 0.144190 | 0.775330 |
| Lactobacillales; Enterococcaceae; | | | | | | | | | |
| Enterococcus; Other | | | | | | | | | |
| Bacteria; Firmicutes; Clostridia; | 20 | 0.011864 | 14.919897 | 0.007296 | 0.018218 | 0.820823 | 0.000489 | 0.002122 | 0.213656 |
| Clostridiales; Lachnospiraceae; | | | | | | | | | |
| Roseburia; Roseburia intestinalis | | | | | | | | | |
| Bacteria; Firmicutes; Clostridia; | 21 | 0.010849 | 9.790054 | 0.008589 | 0.017120 | 0.903148 | 0.000877 | 0.003080 | 0.431718 |
| Clostridiales; Lachnospiraceae; | | | | | | | | | |
| Anaerostipes; Other | | | | | | | | | |
| Bacteria; Firmicutes; Clostridia; | 22 | 0.010778 | 2.754460 | 0.045474 | 0.037841 | 0.968523 | 0.016509 | 0.039130 | 0.850220 |
| Clostridiales; Lachnospiraceae; | | | | | | | | | |
| Other; Other | | | | | | | | | |
| Bacteria; Firmicutes; Clostridia; | 23 | 0.010363 | 8.786461 | 0.003015 | 0.004631 | 0.685230 | 0.000343 | 0.002312 | 0.129956 |
| Clostridiales; Lachnospiraceae; | | | | | | | | | |
| Coprococcus; Coprococcus comes | | | | | | | | | |
| Bacteria; Bacteroidetes; Bacteroidia; | 24 | 0.010293 | 2.672755 | 0.002594 | 0.006865 | 0.682809 | 0.000971 | 0.006250 | 0.101322 |
| Bacteroidales; Porphyromonadaceae; | | | | | | | | | |
| Barnesiella; Other | | | | | | | | | |
| Bacteria; Firmicutes; Bacilli; | 25 | 0.010209 | 11.280681 | 0.004296 | 0.009065 | 0.685230 | 0.000381 | 0.002913 | 0.101322 |
| Lactobacillales; Lactobacillaceae; | | | | | | | | | |
| Lactobacillus; Lactobacillus rogosae | | | | | | | | | |
| Bacteria; Firmicutes; Clostridia; | 26 | 0.010178 | 0.103434 | 0.000069 | 0.001162 | 0.016949 | 0.000667 | 0.003204 | 0.458150 |
| Clostridiales; Peptostreptococcaceae; | | | | | | | | | |
| Clostridium XI; Other | | | | | | | | | |
| Bacteria; Firmicutes; Clostridia; | 27 | 0.010093 | 5.279170 | 0.006442 | 0.014729 | 0.920097 | 0.001220 | 0.004265 | 0.345815 |
| Clostridiales; Peptostreptococcaceae; | | | | | | | | | |
| Romboutsia; Romboutsia timonensis | | | | | | | | | |
| Bacteria; Firmicutes; Other; Other; | 28 | 0.009571 | 7.217686 | 0.001140 | 0.001970 | 0.869249 | 0.000158 | 0.000446 | 0.422907 |
| Other; Other; Other | | | | | | | | | |
| Bacteria; Firmicutes; Clostridia; | 29 | 0.009366 | 1.717128 | 0.031067 | 0.038784 | 0.978208 | 0.018092 | 0.049472 | 0.491189 |
| Clostridiales; Ruminococcaceae; | | | | | | | | | |
| Faecalibacterium; Other | | | | | | | | | |
| Bacteria; Firmicutes; Clostridia; | 30 | 0.008957 | 1.711171 | 0.005693 | 0.008644 | 0.772397 | 0.003327 | 0.019940 | 0.207048 |
| Clostridiales; Ruminococcaceae; | | | | | | | | | |
| Gemmiger; Gemmiger formicilis | | | | | | | | | |
| Bacteria; Candidatus Saccharibacteria; | 31 | 0.008865 | 162.333853 | 0.000232 | 0.001213 | 0.295400 | 0.000001 | 0.000014 | 0.011013 |
| Saccharibacteria_genera_incertae_sedis; | | | | | | | | | |
| Other; Other; Other; Other | | | | | | | | | |
| Bacteria; Firmicutes; Clostridia; | 32 | 0.008829 | 11.444937 | 0.005117 | 0.010630 | 0.728814 | 0.000447 | 0.002306 | 0.145374 |
| Clostridiales; Eubacteriaceae; | | | | | | | | | |
| Eubacterium; [Eubacterium] | | | | | | | | | |
| eligens | | | | | | | | | |
| Bacteria; Bacteroidetes; Bacteroidia; | 33 | 0.008294 | 97.826414 | 0.017116 | 0.050016 | 0.539952 | 0.000175 | 0.001986 | 0.039648 |
| Bacteroidales; Bacteroidaceae; | | | | | | | | | |
| Bacteroides; Bacteroides plebeius | | | | | | | | | |
| Bacteria; Firmicutes; Clostridia; | 34 | 0.008142 | 5.048500 | 0.030591 | 0.053003 | 0.924939 | 0.006060 | 0.013562 | 0.709251 |
| Clostridiales; Lachnospiraceae; | | | | | | | | | |
| Blautia; Other | | | | | | | | | |
| Bacteria; Firmicutes; Clostridia; | 35 | 0.008129 | 5.489082 | 0.003722 | 0.006315 | 0.799031 | 0.000678 | 0.005538 | 0.237885 |
| Clostridiales; Lachnospiraceae; | | | | | | | | | |
| Roseburia; Roseburia faecis | | | | | | | | | |
| Bacteria; Firmicutes; Clostridia; | 36 | 0.008057 | 7.786071 | 0.005283 | 0.010157 | 0.767554 | 0.000678 | 0.003388 | 0.213656 |
| Clostridiales; Lachnospiraceae; | | | | | | | | | |
| Blautia; Blautia obeum | | | | | | | | | |
| Bacteria; Firmicutes; Clostridia; | 37 | 0.007936 | 4.220011 | 0.021757 | 0.027256 | 0.934625 | 0.005156 | 0.013758 | 0.632159 |
| Clostridiales; Lachnospiraceae; | | | | | | | | | |
| Lachnospiracea_incertae_sedis; Other | | | | | | | | | |
| Bacteria; Firmicutes; Clostridia; | 38 | 0.007862 | 19.531980 | 0.000875 | 0.001532 | 0.578692 | 0.000045 | 0.000265 | 0.066079 |
| Clostridiales; Lachnospiraceae; | | | | | | | | | |
| Coprococcus; Coprococcus catus | | | | | | | | | |
| Bacteria; Proteobacteria; | 39 | 0.007734 | 38.633138 | 0.000310 | 0.000933 | 0.302663 | 0.000008 | 0.000040 | 0.070485 |
| Betaproteobacteria; Burkholderiales; | | | | | | | | | |
| Other; Other; Other | | | | | | | | | |
| Bacteria; Firmicutes; Clostridia; | 40 | 0.007705 | 2.980714 | 0.015597 | 0.018427 | 0.934625 | 0.005233 | 0.027016 | 0.561674 |
| Clostridiales; Ruminococcaceae; | | | | | | | | | |
| Other; Other | | | | | | | | | |
| Bacteria; Bacteroidetes; Bacteroidia; | 41 | 0.007688 | 11.365246 | 0.038999 | 0.113177 | 0.685230 | 0.003431 | 0.031801 | 0.176211 |
| Bacteroidales; Prevotellaceae; | | | | | | | | | |
| Prevotella; Prevotella copri | | | | | | | | | |
| Bacteria; Proteobacteria; | 42 | 0.007392 | 5366.285265 | 0.000554 | 0.001748 | 0.096852 | 0.000000 | 0.000002 | 0.002203 |
| Betaproteobacteria; Burkholderiales; | | | | | | | | | |
| Burkholderiaceae; Other; Other | | | | | | | | | |
| Bacteria; Proteobacteria; | 43 | 0.007034 | NA - Div | 0.005094 | 0.028774 | 0.099274 | 0.000000 | 0.000000 | 0.000000 |
| Alphaproteobacteria; Caulobacterales; | | | by 0 | | | | | | |
| Caulobacteraceae; Caulobacter; | | | | | | | | | |
| Caulobacter segnis | | | | | | | | | |
| Bacteria; Proteobacteria; | 44 | 0.006272 | NA - Div | 0.000283 | 0.001381 | 0.101695 | 0.000000 | 0.000000 | 0.000000 |
| Alphaproteobacteria; Caulobacterales; | | | by 0 | | | | | | |
| Caulobacteraceae; Caulobacter; Other | | | | | | | | | |
| Bacteria; Proteobacteria; | 45 | 0.006261 | 21616.489716 | 0.087316 | 0.269557 | 0.099274 | 0.000004 | 0.000034 | 0.015419 |
| Betaproteobacteria; Burkholderiales; | | | | | | | | | |
| Burkholderiaceae; Burkholderia; Other | | | | | | | | | |
| Bacteria; Proteobacteria; | 46 | 0.006218 | 19.741194 | 0.000274 | 0.000849 | 0.210654 | 0.000014 | 0.000122 | 0.059471 |
| Betaproteobacteria; Other; Other; | | | | | | | | | |
| Other; Other | | | | | | | | | |
| Bacteria; Proteobacteria; | 47 | 0.006198 | NA - Div | 0.000010 | 0.000036 | 0.096852 | 0.000000 | 0.000000 | 0.000000 |
| Betaproteobacteria; Burkholderiales; | | | by 0 | | | | | | |
| Burkholderiaceae; Burkholderia; | | | | | | | | | |
| Burkholderia ambifaria | | | | | | | | | |
| Bacteria; Bacteroidetes; Bacteroidia; | 48 | 0.006085 | 12.753613 | 0.041010 | 0.107593 | 0.777240 | 0.003216 | 0.027796 | 0.297357 |
| Bacteroidales; Prevotellaceae; | | | | | | | | | |
| Prevotella; Other | | | | | | | | | |
| Bacteria; Proteobacteria; | 49 | 0.005983 | 16.904024 | 0.000028 | 0.000177 | 0.133172 | 0.000002 | 0.000018 | 0.008811 |
| Betaproteobacteria; Burkholderiales; | | | | | | | | | |
| Comamonadaceae; Other; Other | | | | | | | | | |
| Bacteria; Proteobacteria; | 50 | 0.005837 | 0.008974 | 0.000024 | 0.000202 | 0.079903 | 0.002672 | 0.010560 | 0.469163 |
| Gammaproteobacteria; | | | | | | | | | |
| Enterobacterales; Other; Other; | | | | | | | | | |
| Other | | | | | | | | | |
| Bacteria; Firmicutes; Clostridia; | 51 | 0.005830 | 2.145134 | 0.004604 | 0.008826 | 0.723971 | 0.002146 | 0.026081 | 0.191630 |
| Clostridiales; Ruminococcaceae; | | | | | | | | | |
| Ruminococcus; Ruminococcus faecis | | | | | | | | | |
| Bacteria; Firmicutes; Bacilli; | 52 | 0.005590 | 0.015396 | 0.000070 | 0.000623 | 0.261501 | 0.004568 | 0.016546 | 0.590308 |
| Lactobacillales; Other; Other; | | | | | | | | | |
| Other | | | | | | | | | |
| Bacteria; Proteobacteria; | 53 | 0.005507 | NA - Div | 0.000002 | 0.000009 | 0.077482 | 0.000000 | 0.000000 | 0.000000 |
| Betaproteobacteria; Burkholderiales; | | | by 0 | | | | | | |
| Burkholderiaceae; Burkholderia; | | | | | | | | | |
| Burkholderia thailandensis | | | | | | | | | |
| Bacteria; Bacteroidetes; Bacteroidia; | 54 | 0.005493 | 1.115759 | 0.000219 | 0.000524 | 0.639225 | 0.000196 | 0.001077 | 0.132159 |
| Bacteroidales; Porphyromonadaceae; | | | | | | | | | |
| Odoribacter; Other | | | | | | | | | |
| Bacteria; Firmicutes; Clostridia; | 55 | 0.005418 | 2.642972 | 0.004107 | 0.005814 | 0.779661 | 0.001554 | 0.009333 | 0.266520 |
| Clostridiales; Lachnospiraceae; | | | | | | | | | |
| Blautia; Blautia faecis | | | | | | | | | |
| Bacteria; Firmicutes; Clostridia; | 56 | 0.005260 | 1.851942 | 0.008033 | 0.011088 | 0.849879 | 0.004338 | 0.022200 | 0.301762 |
| Clostridiales; Lachnospiraceae; | | | | | | | | | |
| Blautia; Blautia luti | | | | | | | | | |
| Bacteria; Bacteroidetes; Bacteroidia; | 57 | 0.005256 | 15.164959 | 0.000849 | 0.001662 | 0.583535 | 0.000056 | 0.000396 | 0.059471 |
| Bacteroidales; Rikenellaceae; | | | | | | | | | |
| Alistipes; Alistipes obesi | | | | | | | | | |
| Bacteria; Bacteroidetes; Bacteroidia; | 58 | 0.005203 | 1.402469 | 0.006580 | 0.020688 | 0.859564 | 0.004691 | 0.027357 | 0.407489 |
| Bacteroidales; Rikenellaceae; | | | | | | | | | |
| Alistipes; Other | | | | | | | | | |
| Bacteria; Firmicutes; Clostridia; | 59 | 0.004845 | 1.906142 | 0.026474 | 0.041908 | 0.820823 | 0.013889 | 0.039999 | 0.533040 |
| Clostridiales; Lachnospiraceae; | | | | | | | | | |
| Blautia; Blautia wexlerae | | | | | | | | | |
| Bacteria; Actinobacteria; | 60 | 0.004588 | 0.091461 | 0.000135 | 0.000423 | 0.401937 | 0.001476 | 0.005130 | 0.486784 |
| Coriobacteriia; Eggerthellales; | | | | | | | | | |
| Eggerthellaceae; Eggerthella; | | | | | | | | | |
| Eggerthella lenta | | | | | | | | | |
| Bacteria; Firmicutes; Clostridia; | 61 | 0.004406 | 90.950753 | 0.001507 | 0.004129 | 0.435835 | 0.000017 | 0.000114 | 0.063877 |
| Clostridiales; Lachnospiraceae; | | | | | | | | | |
| Blautia; Blautia stercoris | | | | | | | | | |
| Bacteria; Firmicutes; Clostridia; | 62 | 0.004254 | 2.088425 | 0.001188 | 0.004845 | 0.714286 | 0.000569 | 0.006346 | 0.213656 |
| Clostridiales; Lachnospiraceae; | | | | | | | | | |
| Roseburia; Roseburia hominis | | | | | | | | | |
| Bacteria; Firmicutes; Clostridia; | 63 | 0.004216 | 2.806961 | 0.000375 | 0.001684 | 0.503632 | 0.000134 | 0.001249 | 0.068282 |
| Clostridiales; Ruminococcaceae; | | | | | | | | | |
| Gemmiger; Other | | | | | | | | | |
| Bacteria; Firmicutes; Clostridia; | 64 | 0.004153 | 11.111505 | 0.007834 | 0.018470 | 0.539952 | 0.000705 | 0.004419 | 0.125551 |
| Clostridiales; Ruminococcaceae; | | | | | | | | | |
| Ruminococcus; Ruminococcus bromii | | | | | | | | | |
| Bacteria; Proteobacteria; | 65 | 0.004114 | 27.234354 | 0.000532 | 0.004233 | 0.118644 | 0.000020 | 0.000179 | 0.050661 |
| Betaproteobacteria; Burkholderiales; | | | | | | | | | |
| Comamonadaceae; Pelomonas; | | | | | | | | | |
| Pelomonas aquatica | | | | | | | | | |
| Bacteria; Firmicutes; Erysipelotrichia; | 66 | 0.004104 | 0.087663 | 0.000444 | 0.003707 | 0.547215 | 0.005062 | 0.019598 | 0.665198 |
| Erysipelotrichales; Erysipelotrichaceae; | | | | | | | | | |
| Erysipelatoclostridium; | | | | | | | | | |
| [Clostridium] innocuum | | | | | | | | | |
| Bacteria; Firmicutes; Clostridia; | 67 | 0.004010 | 27.226182 | 0.000738 | 0.002749 | 0.450363 | 0.000027 | 0.000160 | 0.085903 |
| Clostridiales; Peptostreptococcaceae; | | | | | | | | | |
| Romboutsia; Other | | | | | | | | | |
| Bacteria; Bacteroidetes; Bacteroidia; | 68 | 0.003987 | 157.437668 | 0.010799 | 0.032470 | 0.433414 | 0.000069 | 0.000843 | 0.039648 |
| Bacteroidales; Bacteroidaceae; | | | | | | | | | |
| Bacteroides; Bacteroides coprocola | | | | | | | | | |
| Bacteria; Proteobacteria; | 69 | 0.003973 | 0.006062 | 0.000012 | 0.000094 | 0.075061 | 0.002035 | 0.008768 | 0.431718 |
| Gammaproteobacteria; Enterobacterales; | | | | | | | | | |
| Enterobacteriaceae; Citrobacter; Other | | | | | | | | | |
| Bacteria; Firmicutes; Clostridia; | 70 | 0.003916 | 4.231989 | 0.002048 | 0.009399 | 0.719128 | 0.000484 | 0.003051 | 0.235683 |
| Clostridiales; Ruminococcaceae; | | | | | | | | | |
| Clostridium IV; Other | | | | | | | | | |
| Bacteria; Firmicutes; Clostridia; | 71 | 0.003898 | 1.696293 | 0.004514 | 0.012317 | 0.876513 | 0.002661 | 0.011396 | 0.466960 |
| Clostridiales; Peptostreptococcaceae; | | | | | | | | | |
| Intestinibacter; Intestinibacter | | | | | | | | | |
| bartlettii | | | | | | | | | |
| Bacteria; Firmicutes; Bacilli; | 72 | 0.003847 | 9.436626 | 0.001743 | 0.006303 | 0.602906 | 0.000185 | 0.000805 | 0.273128 |
| Lactobacillales; Streptococcaceae; | | | | | | | | | |
| Streptococcus; Streptococcus | | | | | | | | | |
| thermophilus | | | | | | | | | |
| Bacteria; Firmicutes; Clostridia; | 73 | 0.003647 | 0.145758 | 0.000853 | 0.002469 | 0.799031 | 0.005855 | 0.013353 | 0.786344 |
| Clostridiales; Lachnospiraceae; | | | | | | | | | |
| Clostridium XIVa; Other | | | | | | | | | |
| Bacteria; Proteobacteria; | 74 | 0.003483 | 0.744367 | 0.001584 | 0.003109 | 0.702179 | 0.002128 | 0.007865 | 0.270925 |
| Deltaproteobacteria; Desulfovibrionales; | | | | | | | | | |
| Desulfovibrionaceae; Bilophila; Other | | | | | | | | | |
| Bacteria; Firmicutes; Clostridia; | 75 | 0.003445 | 7.014507 | 0.000345 | 0.001085 | 0.542373 | 0.000049 | 0.000345 | 0.079295 |
| Clostridiales; Ruminococcaceae; | | | | | | | | | |
| Oscillibacter; Other | | | | | | | | | |
| Bacteria; Firmicutes; Clostridia; | 76 | 0.003402 | 0.070129 | 0.000759 | 0.002089 | 0.716707 | 0.010829 | 0.029193 | 0.711454 |
| Clostridiales; Lachnospiraceae; | | | | | | | | | |
| Lachnoclostridium; [Clostridium] | | | | | | | | | |
| bolteae | | | | | | | | | |
| Bacteria; Firmicutes; Erysipelotrichia; | 77 | 0.003390 | 0.135249 | 0.000976 | 0.004688 | 0.493947 | 0.007217 | 0.022214 | 0.658590 |
| Erysipelotrichales; Erysipelotrichaceae; | | | | | | | | | |
| Erysipelatoclostridium; | | | | | | | | | |
| Erysipelatoclostridium ramosum | | | | | | | | | |
| Bacteria; Bacteroidetes; Bacteroidia; | 78 | 0.003233 | 3.500150 | 0.007788 | 0.014441 | 0.692494 | 0.002225 | 0.009918 | 0.209251 |
| Bacteroidales; Rikenellaceae; | | | | | | | | | |
| Alistipes; Alistipes putredinis | | | | | | | | | |
| Bacteria; Bacteroidetes; Bacteroidia; | 79 | 0.003162 | 0.304767 | 0.000225 | 0.000559 | 0.401937 | 0.000739 | 0.009585 | 0.037445 |
| Bacteroidales; Barnesiellaceae; | | | | | | | | | |
| Coprobacter; Coprobacter fastidiosus | | | | | | | | | |
| Bacteria; Bacteroidetes; Bacteroidia; | 80 | 0.003158 | 5.121584 | 0.000424 | 0.001182 | 0.472155 | 0.000083 | 0.000540 | 0.057269 |
| Bacteroidales; Rikenellaceae; | | | | | | | | | |
| Alistipes; Alistipes ihumii | | | | | | | | | |
| Bacteria; Firmicutes; Clostridia; | 81 | 0.002980 | 6.485011 | 0.001360 | 0.003041 | 0.452785 | 0.000210 | 0.001505 | 0.046256 |
| Clostridiales; Ruminococcaceae; | | | | | | | | | |
| Ruminococcus; Ruminococcus callidus | | | | | | | | | |
| Bacteria; Firmicutes; Clostridia; | 82 | 0.002965 | 0.029405 | 0.000040 | 0.000243 | 0.237288 | 0.001372 | 0.005976 | 0.385463 |
| Clostridiales; Clostridiaceae; | | | | | | | | | |
| Hungatella; Hungatella effluvii | | | | | | | | | |
| Bacteria; Proteobacteria; | 83 | 0.002963 | 137.878020 | 0.000118 | 0.001758 | 0.099274 | 0.000001 | 0.000010 | 0.008811 |
| Alphaproteobacteria; Rhizobiales; | | | | | | | | | |
| Phyllobacteriaceae; Phyllobacterium; | | | | | | | | | |
| Other | | | | | | | | | |
| Bacteria; Proteobacteria; | 84 | 0.002874 | 0.106988 | 0.000054 | 0.000399 | 0.288136 | 0.000501 | 0.001112 | 0.475771 |
| Gammaproteobacteria; Other; Other; | | | | | | | | | |
| Other; Other | | | | | | | | | |
| Bacteria; Actinobacteria; | 85 | 0.002843 | 1.421827 | 0.001018 | 0.003735 | 0.690073 | 0.000716 | 0.003769 | 0.240088 |
| Actinobacteria; Bifidobacteriales; | | | | | | | | | |
| Bifidobacteriaceae; Bifidobacterium; | | | | | | | | | |
| Bifidobacterium longum | | | | | | | | | |
| Bacteria; Firmicutes; Clostridia; | 86 | 0.002704 | 0.490765 | 0.003396 | 0.005044 | 0.891041 | 0.006920 | 0.021979 | 0.466960 |
| Clostridiales; Lachnospiraceae; | | | | | | | | | |
| Ruminococcus2; Other | | | | | | | | | |
| Bacteria; Firmicutes; Negativicutes; | 87 | 0.002687 | 0.031220 | 0.000238 | 0.001404 | 0.341404 | 0.007608 | 0.034840 | 0.504405 |
| Veillonellales; Veillonellaceae; | | | | | | | | | |
| Veillonella; Veillonella parvula | | | | | | | | | |
| Bacteria; Actinobacteria; | 88 | 0.002663 | 4.095545 | 0.004684 | 0.011625 | 0.699758 | 0.001144 | 0.007492 | 0.244493 |
| Actinobacteria; Bifidobacteriales; | | | | | | | | | |
| Bifidobacteriaceae; Bifidobacterium; | | | | | | | | | |
| Bifidobacterium adolescentis | | | | | | | | | |
| Bacteria; Firmicutes; Erysipelotrichia; | 89 | 0.002544 | 0.248360 | 0.000046 | 0.000161 | 0.341404 | 0.000186 | 0.001803 | 0.068282 |
| Erysipelotrichales; Erysipelotrichaceae; | | | | | | | | | |
| Solobacterium; Solobacterium moorei | | | | | | | | | |
| Bacteria; Bacteroidetes; Bacteroidia; | 90 | 0.002539 | 0.739000 | 0.051954 | 0.062313 | 0.961259 | 0.070303 | 0.112647 | 0.909692 |
| Bacteroidales; Bacteroidaceae; | | | | | | | | | |
| Bacteroides; Other | | | | | | | | | |
| Bacteria; Firmicutes; Bacilli; | 91 | 0.002440 | 0.040370 | 0.000744 | 0.009319 | 0.476998 | 0.018423 | 0.083583 | 0.643172 |
| Lactobacillales; Lactobacilloae; | | | | | | | | | |
| Lactobacillus; Other | | | | | | | | | |
| Bacteria; Firmicutes; Clostridia; | 92 | 0.002424 | 0.185342 | 0.002506 | 0.011498 | 0.600484 | 0.013521 | 0.029521 | 0.647577 |
| Clostridiales; Lachnospiraceae; | | | | | | | | | |
| Blautia; [Ruminococcus] | | | | | | | | | |
| gnavus | | | | | | | | | |
| Bacteria; Bacteroidetes; Bacteroidia; | 93 | 0.002411 | 1.338446 | 0.001846 | 0.005767 | 0.607748 | 0.001379 | 0.012053 | 0.191630 |
| Bacteroidales; Rikenellaceae; | | | | | | | | | |
| Alistipes; Alistipes finegoldii | | | | | | | | | |
| Bacteria; Bacteroidetes; Bacteroidia; | 94 | 0.002347 | 0.956387 | 0.002441 | 0.005129 | 0.677966 | 0.002552 | 0.035517 | 0.187225 |
| Bacteroidales; Rikenellaceae; | | | | | | | | | |
| Alistipes; Alistipes shahii | | | | | | | | | |
| Bacteria; Proteobacteria; | 95 | 0.002339 | 2.594337 | 0.000004 | 0.000023 | 0.058111 | 0.000001 | 0.000016 | 0.008811 |
| Alphaproteobacteria; Caulobacterales; | | | | | | | | | |
| Caulobacteraceae; Other; Other | | | | | | | | | |
| Bacteria; Firmicutes; Clostridia; | 96 | 0.002326 | 695.608879 | 0.003620 | 0.011337 | 0.382567 | 0.000005 | 0.000053 | 0.015419 |
| Clostridiales; Lachnospiraceae; | | | | | | | | | |
| Coprococcus; Coprococcus eutactus | | | | | | | | | |
| Bacteria; Firmicutes; Bacilli; | 97 | 0.002271 | 0.005918 | 0.000001 | 0.000015 | 0.007264 | 0.000148 | 0.000493 | 0.312775 |
| Lactobacillales; Enterococcaceae; | | | | | | | | | |
| Enterococcus; Enterococcus | | | | | | | | | |
| saccharolyticus | | | | | | | | | |
| Bacteria; Bacteroidetes; Bacteroidia; | 98 | 0.002243 | 3.784396 | 0.000258 | 0.000824 | 0.443099 | 0.000068 | 0.000612 | 0.055066 |
| Bacteroidales; Rikenellaceae; | | | | | | | | | |
| Alistipes; Alistipes indistinctus | | | | | | | | | |
| Bacteria; Actinobacteria; | 99 | 0.002226 | 0.067565 | 0.000029 | 0.000122 | 0.208232 | 0.000435 | 0.001791 | 0.281938 |
| Actinobacteria; Coriobacteriales; | | | | | | | | | |
| Coriobacteriaceae; Eggerthella; Other | | | | | | | | | |
| Bacteria; Firmicutes; Clostridia; | 100 | 0.002209 | 5.591039 | 0.001891 | 0.008662 | 0.590799 | 0.000338 | 0.002766 | 0.273128 |
| Clostridiales; Peptostreptococcaceae; | | | | | | | | | |
| Intestinibacter; Other | | | | | | | | | |

IBS = Irritable Bowel Syndrome;
CDI = Clostridiodes difficile Infection;
RFFR = Random Forest Feature Rank;
# = Rank;
MDA = Mean Decrease Accuracy;
FC = Fold Change;
M = Mean;
SD = Standard Deviation;
DP = Detection Prevalence;

TABLE 11
IBS vs IBD delineation features - (taxonomy)

| Feature | | | | | | | | | |
|---|---|---|---|---|---|---|---|---|---|
| (Taxonomic order: Kingdom; | | | FC | Abundance | | | Abundance | | |
| Phylum; Class; Order; | RFFR | | IBS vs | (IBS) | | | (IBD) | | |
| Family; Genus; Species) | # | MDA | IBD | M | SD | DP | M | SD | DP |
| Bacteria; Firmicutes; Clostridia; | 1 | 0.019964 | 2.064120 | 0.006442 | 0.014729 | 0.920097 | 0.003121 | 0.018450 | 0.360952 |
| Clostridiales; Peptostreptococcaceae; | | | | | | | | | |
| Romboutsia; Romboutsia timonensis | | | | | | | | | |
| Bacteria; Proteobacteria; | 2 | 0.018791 | 0.281149 | 0.000121 | 0.000994 | 0.394673 | 0.000431 | 0.010385 | 0.078095 |
| Gammaproteobacteria; Pseudomonadales; | | | | | | | | | |
| Pseudomonadaceae; Pseudomonas; Other | | | | | | | | | |
| Bacteria; Firmicutes; Clostridia; | 3 | 0.017194 | 3.375668 | 0.019894 | 0.026907 | 0.869249 | 0.005893 | 0.019791 | 0.630476 |
| Clostridiales; Ruminococcaceae; | | | | | | | | | |
| Faecalibacterium; Faecalibacterium | | | | | | | | | |
| prausnitzii | | | | | | | | | |
| Bacteria; Other; Other; Other; | 4 | 0.016740 | 0.264096 | 0.005521 | 0.011388 | 0.966102 | 0.020907 | 0.037823 | 0.939048 |
| Other; Other; Other | | | | | | | | | |
| Bacteria; Firmicutes; Clostridia; | 5 | 0.014815 | 2.330675 | 0.026790 | 0.042600 | 0.927361 | 0.011495 | 0.034475 | 0.402857 |
| Clostridiales; Lachnospiraceae; | | | | | | | | | |
| Not Available; [Eubacterium] | | | | | | | | | |
| rectale | | | | | | | | | |
| Bacteria; Bacteroidetes; Bacteroidia; | 6 | 0.013814 | 3.890844 | 0.003581 | 0.009164 | 0.803874 | 0.000920 | 0.003588 | 0.313333 |
| Bacteroidales; Bacteroidaceae; | | | | | | | | | |
| Bacteroides; Bacteroides xylanisolvens | | | | | | | | | |
| Bacteria; Firmicutes; Bacilli; | 7 | 0.012597 | 1.577191 | 0.000049 | 0.000115 | 0.421308 | 0.000031 | 0.000142 | 0.112381 |
| Lactobacillales; Carnobacteriaceae; | | | | | | | | | |
| Granulicatella; Other | | | | | | | | | |
| Bacteria; Actinobacteria; | 8 | 0.012353 | 0.194215 | 0.001370 | 0.005518 | 0.351090 | 0.007053 | 0.023275 | 0.728571 |
| Coriobacteriia; Coriobacteriales; | | | | | | | | | |
| Coriobacteriaceae; Collinsella; | | | | | | | | | |
| Collinsella aerofaciens | | | | | | | | | |
| Bacteria; Firmicutes; Negativicutes; | 9 | 0.011956 | 0.015831 | 0.000013 | 0.000172 | 0.050847 | 0.000838 | 0.008203 | 0.515238 |
| Veillonellales; Veillonellaceae; | | | | | | | | | |
| Veillonella; Other | | | | | | | | | |
| Bacteria; Bacteroidetes; Bacteroidia; | 10 | 0.011201 | 1.777263 | 0.040437 | 0.064921 | 0.842615 | 0.022753 | 0.060571 | 0.389524 |
| Bacteroidales; Bacteroidaceae; | | | | | | | | | |
| Bacteroides; Bacteroides dorei | | | | | | | | | |
| Bacteria; Firmicutes; Clostridia; | 11 | 0.010434 | 1.847237 | 0.003722 | 0.006315 | 0.799031 | 0.002015 | 0.008784 | 0.342857 |
| Clostridiales; Lachnospiraceae; | | | | | | | | | |
| Roseburia; Roseburia faecis | | | | | | | | | |
| Bacteria; Firmicutes; Bacilli; | 12 | 0.010179 | 1.143292 | 0.000246 | 0.000996 | 0.559322 | 0.000215 | 0.001645 | 0.199048 |
| Lactobacillales; Streptococcaceae; | | | | | | | | | |
| Streptococcus; Streptococcus | | | | | | | | | |
| parasanguinis | | | | | | | | | |
| Bacteria; Firmicutes; Clostridia; | 13 | 0.009881 | 0.000579 | 0.000001 | 0.000006 | 0.016949 | 0.001136 | 0.006502 | 0.476190 |
| Clostridiales; Lachnospiraceae; | | | | | | | | | |
| Lachnoclostridium; Other | | | | | | | | | |
| Bacteria; Firmicutes; Clostridia; | 14 | 0.009195 | 0.034059 | 0.000156 | 0.000820 | 0.363196 | 0.004586 | 0.025790 | 0.334286 |
| Clostridiales; Lachnospiraceae; | | | | | | | | | |
| Blautia; Blautia hominis | | | | | | | | | |
| Bacteria; Firmicutes; Clostridia; | 15 | 0.008216 | 1.857272 | 0.001605 | 0.002634 | 0.750605 | 0.000864 | 0.003153 | 0.328571 |
| Clostridiales; Ruminococcaceae; | | | | | | | | | |
| Agathobaculum; Agathobaculum | | | | | | | | | |
| butyriciproducens | | | | | | | | | |
| Bacteria; Actinobacteria; | 16 | 0.008119 | 0.421662 | 0.001627 | 0.007075 | 0.292978 | 0.003859 | 0.011240 | 0.681905 |
| Actinobacteria; Coriobacteriales; | | | | | | | | | |
| Coriobacteriaceae; Collinsella; | | | | | | | | | |
| Other | | | | | | | | | |
| Bacteria; Proteobacteria; | 17 | 0.008009 | 0.001191 | 0.000024 | 0.000202 | 0.079903 | 0.020133 | 0.079882 | 0.538095 |
| Gammaproteobacteria; Enterobacterales; | | | | | | | | | |
| Other; Other; Other | | | | | | | | | |
| Bacteria; Firmicutes; Clostridia; | 18 | 0.008001 | 0.770941 | 0.031067 | 0.038784 | 0.978208 | 0.040297 | 0.054719 | 0.911429 |
| Clostridiales; Ruminococcaceae; | | | | | | | | | |
| Faecalibacterium; Other | | | | | | | | | |
| Bacteria; Firmicutes; Clostridia; | 19 | 0.007947 | 15.250702 | 0.001507 | 0.004129 | 0.435835 | 0.000099 | 0.000607 | 0.167619 |
| Clostridiales; Lachnospiraceae; | | | | | | | | | |
| Blautia; Blautia stercoris | | | | | | | | | |
| Bacteria; Candidatus Saccharibacteria; | 20 | 0.007790 | 21.228866 | 0.000232 | 0.001213 | 0.295400 | 0.000011 | 0.000057 | 0.135238 |
| Saccharibacteria_genera_incertae_sedis; | | | | | | | | | |
| Other; Other; Other; Other | | | | | | | | | |
| Bacteria; Bacteroidetes; Bacteroidia; | 21 | 0.007326 | 5.471413 | 0.017116 | 0.050016 | 0.539952 | 0.003128 | 0.021947 | 0.278095 |
| Bacteroidales; Bacteroidaceae; | | | | | | | | | |
| Bacteroides; Bacteroides plebeius | | | | | | | | | |
| Bacteria; Bacteroidetes; Bacteroidia; | 22 | 0.007307 | 6.064441 | 0.000229 | 0.001087 | 0.322034 | 0.000038 | 0.000392 | 0.051429 |
| Bacteroidales; Bacteroidaceae; | | | | | | | | | |
| Bacteroides; Bacteroides nordii | | | | | | | | | |
| Bacteria; Firmicutes; Clostridia; | 23 | 0.007244 | 3.259107 | 0.004722 | 0.010883 | 0.857143 | 0.001449 | 0.002690 | 0.775238 |
| Clostridiales; Lachnospiraceae; | | | | | | | | | |
| Fusicatenibacter; Other | | | | | | | | | |
| Bacteria; Firmicutes; Bacilli; | 24 | 0.007204 | 1.641026 | 0.001743 | 0.006303 | 0.602906 | 0.001062 | 0.026362 | 0.294286 |
| Lactobacillales; Streptococcaceae; | | | | | | | | | |
| Streptococcus; Streptococcus | | | | | | | | | |
| thermophilus | | | | | | | | | |
| Bacteria; Firmicutes; Clostridia; | 25 | 0.007050 | 1.052566 | 0.005693 | 0.008644 | 0.772397 | 0.005409 | 0.016380 | 0.690476 |
| Clostridiales; Ruminococcaceae; | | | | | | | | | |
| Gemmiger; Gemmiger formicilis | | | | | | | | | |
| Bacteria; Bacteroidetes; Bacteroidia; | 26 | 0.006989 | 2.975546 | 0.002235 | 0.007078 | 0.760291 | 0.000751 | 0.004555 | 0.337143 |
| Bacteroidales; Bacteroidaceae; | | | | | | | | | |
| Bacteroides; Bacteroides ovatus | | | | | | | | | |
| Bacteria; Bacteroidetes; Other; | 27 | 0.006981 | 0.409003 | 0.000658 | 0.008037 | 0.554479 | 0.001610 | 0.004345 | 0.674286 |
| Other; Other; Other; Other | | | | | | | | | |
| Bacteria; Firmicutes; Clostridia; | 28 | 0.006729 | 3.151968 | 0.013466 | 0.023548 | 0.791768 | 0.004272 | 0.012950 | 0.694286 |
| Clostridiales; Ruminococcaceae; | | | | | | | | | |
| Ruminococcus; Other | | | | | | | | | |
| Bacteria; Firmicutes; Clostridia; | 29 | 0.006553 | NA - Div | 0.000280 | 0.002139 | 0.159806 | 0.000000 | 0.000000 | 0.000000 |
| Clostridiales; Ruminococcaceae; | | | by 0 | | | | | | |
| Not Available; [Eubacterium] | | | | | | | | | |
| siraeum | | | | | | | | | |
| Bacteria; Bacteroidetes; Bacteroidia; | 30 | 0.006250 | 4.220951 | 0.007573 | 0.020157 | 0.556901 | 0.001794 | 0.008110 | 0.190476 |
| Bacteroidales; Bacteroidaceae; | | | | | | | | | |
| Bacteroides; Bacteroides massiliensis | | | | | | | | | |
| Bacteria; Bacteroidetes; Bacteroidia; | 31 | 0.006149 | 5.462881 | 0.004005 | 0.017127 | 0.535109 | 0.000733 | 0.006500 | 0.179048 |
| Bacteroidales; Bacteroidaceae; | | | | | | | | | |
| Bacteroides; Bacteroides | | | | | | | | | |
| cellulosilyticus | | | | | | | | | |
| Bacteria; Proteobacteria; | 32 | 0.005748 | 45122.221500 | 0.000554 | 0.001748 | 0.096852 | 0.000000 | 0.000000 | 0.000952 |
| Betaproteobacteria; Burkholderiales; | | | | | | | | | |
| Burkholderiaceae; Other; Other | | | | | | | | | |
| Bacteria; Proteobacteria; | 33 | 0.005548 | NA - Div | 0.000283 | 0.001381 | 0.101695 | 0.000000 | 0.000000 | 0.000000 |
| Alphaproteobacteria; Caulobacterales; | | | by 0 | | | | | | |
| Caulobacteraceae; Caulobacter; Other | | | | | | | | | |
| Bacteria; Firmicutes; Bacilli; | 34 | 0.005455 | 2.190168 | 0.000725 | 0.002702 | 0.256659 | 0.000331 | 0.004144 | 0.036190 |
| Lactobacillales; Streptococcaceae; | | | | | | | | | |
| Streptococcus; Streptococcus | | | | | | | | | |
| salivarius | | | | | | | | | |
| Bacteria; Firmicutes; Clostridia; | 35 | 0.005382 | 1.226793 | 0.030591 | 0.053003 | 0.924939 | 0.024936 | 0.047896 | 0.984762 |
| Clostridiales; Lachnospiraceae; | | | | | | | | | |
| Blautia; Other | | | | | | | | | |
| Bacteria; Proteobacteria; | 36 | 0.005373 | 2735.033422 | 0.000532 | 0.004233 | 0.118644 | 0.000000 | 0.000003 | 0.006667 |
| Betaproteobacteria; Burkholderiales; | | | | | | | | | |
| Comamonadaceae; Pelomonas; | | | | | | | | | |
| Pelomonas aquatica | | | | | | | | | |
| Bacteria; Firmicutes; Clostridia; | 37 | 0.005362 | 1.139082 | 0.005954 | 0.013082 | 0.893462 | 0.005227 | 0.008361 | 0.887619 |
| Clostridiales; Lachnospiraceae; | | | | | | | | | |
| Roseburia; Other | | | | | | | | | |
| Bacteria; Actinobacteria; | 38 | 0.005163 | 0.132537 | 0.000011 | 0.000045 | 0.138015 | 0.000082 | 0.000245 | 0.545714 |
| Actinobacteria; Bifidobacteriales; | | | | | | | | | |
| Bifidobacteriaceae; Other; Other | | | | | | | | | |
| Bacteria; Firmicutes; Clostridia; | 39 | 0.005043 | 0.304522 | 0.002506 | 0.011498 | 0.600484 | 0.008229 | 0.028839 | 0.841905 |
| Clostridiales; Lachnospiraceae; | | | | | | | | | |
| Blautia; [Ruminococcus] gnavus | | | | | | | | | |
| Bacteria; Firmicutes; Other; Other; | 40 | 0.004982 | 0.644213 | 0.001140 | 0.001970 | 0.869249 | 0.001770 | 0.003186 | 0.906667 |
| Other; Other; Other | | | | | | | | | |
| Bacteria; Proteobacteria; | 41 | 0.004975 | NA - Div | 0.087316 | 0.269557 | 0.099274 | 0.000000 | 0.000000 | 0.000000 |
| Betaproteobacteria; Burkholderiales; | | | by 0 | | | | | | |
| Burkholderiaceae; Burkholderia; Other | | | | | | | | | |
| Bacteria; Proteobacteria; | 42 | 0.004975 | 11938.331577 | 0.005094 | 0.028774 | 0.099274 | 0.000000 | 0.000009 | 0.007619 |
| Alphaproteobacteria; Caulobacterales; | | | | | | | | | |
| Caulobacteraceae; Caulobacter; | | | | | | | | | |
| Caulobacter segnis | | | | | | | | | |
| Bacteria; Firmicutes; Clostridia; | 43 | 0.004886 | 1.057123 | 0.026474 | 0.041908 | 0.820823 | 0.025044 | 0.061261 | 0.471429 |
| Clostridiales; Lachnospiraceae; | | | | | | | | | |
| Blautia; Blautia wexlerae | | | | | | | | | |
| Bacteria; Actinobacteria; | 44 | 0.004878 | 0.254780 | 0.005829 | 0.020871 | 0.799031 | 0.022880 | 0.052680 | 0.916190 |
| Actinobacteria; Bifidobacteriales; | | | | | | | | | |
| Bifidobacteriaceae; Bifidobacterium; | | | | | | | | | |
| Other | | | | | | | | | |
| Bacteria; Bacteroidetes; Bacteroidia; | 45 | 0.004668 | 1.699561 | 0.001521 | 0.002372 | 0.799031 | 0.000895 | 0.001940 | 0.653333 |
| Bacteroidales; Odoribacteraceae; | | | | | | | | | |
| Odoribacter; Odoribacter splanchnicus | | | | | | | | | |
| Bacteria; Firmicutes; Clostridia; | 46 | 0.004646 | 2.126594 | 0.008589 | 0.017120 | 0.903148 | 0.004039 | 0.011111 | 0.932381 |
| Clostridiales; Lachnospiraceae; | | | | | | | | | |
| Anaerostipes; Other | | | | | | | | | |
| Bacteria; Proteobacteria; | 47 | 0.004596 | NA - Div | 0.000010 | 0.000036 | 0.096852 | 0.000000 | 0.000000 | 0.000000 |
| Betaproteobacteria; Burkholderiales; | | | by 0 | | | | | | |
| Burkholderiaceae; Burkholderia; | | | | | | | | | |
| Burkholderia ambifaria | | | | | | | | | |
| Bacteria; Firmicutes; Clostridia; | 48 | 0.004564 | 77.864250 | 0.000246 | 0.001183 | 0.147700 | 0.000003 | 0.000049 | 0.004762 |
| Clostridiales; Eubacteriaceae; | | | | | | | | | |
| Eubacterium; Eubacterium ventriosum | | | | | | | | | |
| Bacteria; Firmicutes; Clostridia; | 49 | 0.004555 | 0.021560 | 0.000008 | 0.000025 | 0.142857 | 0.000350 | 0.000968 | 0.463810 |
| Clostridiales; Ruminococcaceae; | | | | | | | | | |
| Intestinimonas; Other | | | | | | | | | |
| Bacteria; Firmicutes; Clostridia; | 50 | 0.004546 | 7.406121 | 0.000254 | 0.001565 | 0.326877 | 0.000034 | 0.000078 | 0.503810 |
| Other; Other; Other; Other | | | | | | | | | |
| Bacteria; Bacteroidetes; Bacteroidia; | 51 | 0.004533 | 0.267350 | 0.000654 | 0.001669 | 0.748184 | 0.002447 | 0.004365 | 0.805714 |
| Bacteroidales; Other; Other; Other | | | | | | | | | |
| Bacteria; Actinobacteria; | 52 | 0.004470 | 0.411025 | 0.004684 | 0.011625 | 0.699758 | 0.011395 | 0.038469 | 0.388571 |
| Actinobacteria; Bifidobacteriales; | | | | | | | | | |
| Bifidobacteriaceae; Bifidobacterium; | | | | | | | | | |
| Bifidobacterium adolescentis | | | | | | | | | |
| Bacteria; Firmicutes; Clostridia; | 53 | 0.004416 | 0.261449 | 0.000759 | 0.002089 | 0.716707 | 0.002905 | 0.011580 | 0.394286 |
| Clostridiales; Lachnospiraceae; | | | | | | | | | |
| Lachnoclostridium; [Clostridium] | | | | | | | | | |
| bolteae | | | | | | | | | |
| Bacteria; Bacteroidetes; | 54 | 0.004262 | 1.162000 | 0.000219 | 0.000524 | 0.639225 | 0.000188 | 0.000586 | 0.511429 |
| Bacteroidia; Bacteroidales; | | | | | | | | | |
| Porphyromonadaceae; Odoribacter; | | | | | | | | | |
| Other | | | | | | | | | |
| Bacteria; Firmicutes; Clostridia; | 55 | 0.004230 | 2.300468 | 0.000875 | 0.001532 | 0.578692 | 0.000380 | 0.001144 | 0.260952 |
| Clostridiales; Lachnospiraceae; | | | | | | | | | |
| Coprococcus; Coprococcus catus | | | | | | | | | |
| Bacteria; Firmicutes; Bacilli; | 56 | 0.004192 | 0.548312 | 0.007294 | 0.018938 | 0.907990 | 0.013303 | 0.034735 | 0.982857 |
| Lactobacillales; Streptococcaceae; | | | | | | | | | |
| Streptococcus; Other | | | | | | | | | |
| Bacteria; Firmicutes; Clostridia; | 57 | 0.004182 | 3.709381 | 0.004088 | 0.010861 | 0.801453 | 0.001102 | 0.003027 | 0.803810 |
| Clostridiales; Peptostreptococcaceae; | | | | | | | | | |
| Other; Other | | | | | | | | | |
| Bacteria; Firmicutes; Clostridia; | 58 | 0.004180 | 0.086268 | 0.000363 | 0.001754 | 0.157385 | 0.004213 | 0.017069 | 0.440000 |
| Clostridiales; Lachnospiraceae; | | | | | | | | | |
| Faecalimonas; Faecalimonas | | | | | | | | | |
| umbilicata | | | | | | | | | |
| Bacteria; Bacteroidetes; Bacteroidia; | 59 | 0.004122 | 1.364547 | 0.038999 | 0.113177 | 0.685230 | 0.028580 | 0.096181 | 0.687619 |
| Bacteroidales; Prevotellaceae; | | | | | | | | | |
| Prevotella; Prevotella copri | | | | | | | | | |
| Bacteria; Proteobacteria; | 60 | 0.004103 | 8.950773 | 0.000114 | 0.000372 | 0.251816 | 0.000013 | 0.000090 | 0.062857 |
| Betaproteobacteria; Burkholderiales; | | | | | | | | | |
| Oxalobacteraceae; Oxalobacter; | | | | | | | | | |
| Oxalobacter formigenes | | | | | | | | | |
| Bacteria; Firmicutes; Clostridia; | 61 | 0.004038 | 1.483212 | 0.004604 | 0.008826 | 0.723971 | 0.003104 | 0.009542 | 0.689524 |
| Clostridiales; Ruminococcaceae; | | | | | | | | | |
| Ruminococcus; Ruminococcus faecis | | | | | | | | | |
| Bacteria; Actinobacteria; | 62 | 0.004000 | 0.212202 | 0.000073 | 0.000244 | 0.331719 | 0.000346 | 0.002140 | 0.632381 |
| Actinobacteria; Coriobacteriales; | | | | | | | | | |
| Coriobacteriaceae; Other; Other | | | | | | | | | |
| Bacteria; Actinobacteria; | 63 | 0.003985 | 0.178684 | 0.000006 | 0.000039 | 0.055690 | 0.000034 | 0.000091 | 0.425714 |
| Actinobacteria; Actinomycetales; | | | | | | | | | |
| Actinomycetaceae; Other; Other | | | | | | | | | |
| Bacteria; Firmicutes; Clostridia; | 64 | 0.003962 | 0.470831 | 0.000853 | 0.002469 | 0.799031 | 0.001812 | 0.004738 | 0.907619 |
| Clostridiales; Lachnospiraceae; | | | | | | | | | |
| Clostridium XIVa; Other | | | | | | | | | |
| Bacteria; Firmicutes; Clostridia; | 65 | 0.003886 | 1.449196 | 0.000072 | 0.000325 | 0.399516 | 0.000050 | 0.000200 | 0.347619 |
| Clostridiales; Not Available; | | | | | | | | | |
| Intestinimonas; Intestinimonas | | | | | | | | | |
| butyriciproducens | | | | | | | | | |
| Bacteria; Firmicutes; Negativicutes; | 66 | 0.003880 | 0.364950 | 0.000238 | 0.001404 | 0.341404 | 0.000651 | 0.015259 | 0.138095 |
| Veillonellales; Veillonellaceae; | | | | | | | | | |
| Veillonella; Veillonella parvula | | | | | | | | | |
| Bacteria; Firmicutes; Clostridia; | 67 | 0.003872 | 0.771959 | 0.045474 | 0.037841 | 0.968523 | 0.058907 | 0.051085 | 0.991429 |
| Clostridiales; Lachnospiraceae; | | | | | | | | | |
| Other; Other | | | | | | | | | |
| Bacteria; Actinobacteria; | 68 | 0.003863 | 0.024864 | 0.000001 | 0.000006 | 0.024213 | 0.000030 | 0.000086 | 0.400952 |
| Actinobacteria; Other; Other; | | | | | | | | | |
| Other; Other | | | | | | | | | |
| Bacteria; Firmicutes; Clostridia; | 69 | 0.003812 | 0.763719 | 0.000037 | 0.000164 | 0.157385 | 0.000048 | 0.000227 | 0.123810 |
| Clostridiales; Ruminococcaceae; | | | | | | | | | |
| Anaeromassilibacillus; | | | | | | | | | |
| Anaeromassilibacillus senegalensis | | | | | | | | | |
| Bacteria; Firmicutes; Clostridia; | 70 | 0.003772 | 0.000000 | 0.000000 | 0.000000 | 0.000000 | 0.000333 | 0.001662 | 0.084762 |
| Clostridiales; Lachnospiraceae; Dorea; | | | | | | | | | |
| Dorea formicigenerans | | | | | | | | | |
| Bacteria; Firmicutes; Clostridia; | 71 | 0.003710 | 1.549459 | 0.021757 | 0.027256 | 0.934625 | 0.014041 | 0.022190 | 0.978095 |
| Clostridiales; Lachnospiraceae; | | | | | | | | | |
| Lachnospiracea_incertae_sedis; | | | | | | | | | |
| Other | | | | | | | | | |
| Bacteria; Bacteroidetes; | 72 | 0.003659 | 1.649128 | 0.001846 | 0.005767 | 0.607748 | 0.001120 | 0.010523 | 0.459048 |
| Bacteroidia; Bacteroidales; | | | | | | | | | |
| Rikenellaceae; Alistipes; | | | | | | | | | |
| Alistipes finegoldii | | | | | | | | | |
| Bacteria; Firmicutes; Clostridia; | 73 | 0.003486 | 0.657294 | 0.015597 | 0.018427 | 0.934625 | 0.023730 | 0.048235 | 0.938095 |
| Clostridiales; Ruminococcaceae; | | | | | | | | | |
| Other; Other | | | | | | | | | |
| Bacteria; Bacteroidetes; | 74 | 0.003476 | 2.230409 | 0.002441 | 0.005129 | 0.677966 | 0.001094 | 0.002698 | 0.564762 |
| Bacteroidia; Bacteroidales; | | | | | | | | | |
| Rikenellaceae; Alistipes; | | | | | | | | | |
| Alistipes shahii | | | | | | | | | |
| Bacteria; Proteobacteria; | 75 | 0.003405 | 0.226553 | 0.000310 | 0.000933 | 0.302663 | 0.001366 | 0.008723 | 0.554286 |
| Betaproteobacteria; Burkholderiales; | | | | | | | | | |
| Other; Other; Other | | | | | | | | | |
| Bacteria; Firmicutes; Clostridia; | 76 | 0.003385 | 0.697033 | 0.000375 | 0.001684 | 0.503632 | 0.000539 | 0.001815 | 0.466667 |
| Clostridiales; Ruminococcaceae; | | | | | | | | | |
| Gemmiger; Other | | | | | | | | | |
| Bacteria; Proteobacteria; | 77 | 0.003342 | 7.653116 | 0.000028 | 0.00077 | 0.133172 | 0.000004 | 0.000053 | 0.043810 |
| Betaproteobacteria; Burkholderiales; | | | | | | | | | |
| Comamonadaceae; Other; Other | | | | | | | | | |
| Bacteria; Bacteroidetes; Bacteroidia; | 78 | 0.003319 | 1.462976 | 0.041010 | 0.107593 | 0.777240 | 0.028032 | 0.087686 | 0.770476 |
| Bacteroidales; Prevotellaceae; | | | | | | | | | |
| Prevotella; Other | | | | | | | | | |
| Bacteria; Firmicutes; Bacilli; | 79 | 0.003301 | 0.224065 | 0.000008 | 0.000030 | 0.104116 | 0.000035 | 0.000166 | 0.180952 |
| Lactobacillales; Carnobacteriaceae; | | | | | | | | | |
| Granulicatella; Granulicatella | | | | | | | | | |
| adiacens | | | | | | | | | |
| Bacteria; Firmicutes; Clostridia; | 80 | 0.003294 | 1.197894 | 0.016406 | 0.016525 | 0.944310 | 0.013696 | 0.018995 | 0.987619 |
| Clostridiales; Other; Other; Other | | | | | | | | | |
| Bacteria; Firmicutes; Negativicutes; | 81 | 0.003268 | NA - Div | 0.000048 | 0.000333 | 0.096852 | 0.000000 | 0.000000 | 0.000000 |
| Veillonellales; Veillonellaceae; | | | by 0 | | | | | | |
| Veillonella; Veillonella infantium | | | | | | | | | |
| Bacteria; Bacteroidetes; Bacteroidia; | 82 | 0.003266 | 14.581027 | 0.000965 | 0.004492 | 0.232446 | 0.000066 | 0.000500 | 0.108571 |
| Bacteroidales; Bacteroidaceae; | | | | | | | | | |
| Bacteroides; | | | | | | | | | |
| Bacteroides thetaiotaomicron | | | | | | | | | |
| Bacteria; Firmicutes; Clostridia; | 83 | 0.003255 | 0.640063 | 0.004107 | 0.005814 | 0.779661 | 0.006417 | 0.015065 | 0.883810 |
| Clostridiales; Lachnospiraceae; | | | | | | | | | |
| Blautia; Blautia faecis | | | | | | | | | |
| Bacteria; Proteobacteria; | 84 | 0.003240 | 0.545020 | 0.000274 | 0.000849 | 0.210654 | 0.000503 | 0.003162 | 0.464762 |
| Betaproteobacteria; Other; | | | | | | | | | |
| Other; Other; Other | | | | | | | | | |
| Bacteria; Proteobacteria; Other; | 85 | 0.003233 | 0.030060 | 0.000020 | 0.000084 | 0.263923 | 0.000675 | 0.002153 | 0.559048 |
| Other; Other; Other; Other | | | | | | | | | |
| Bacteria; Bacteroidetes; | 86 | 0.003210 | 41.417105 | 0.000009 | 0.000033 | 0.121065 | 0.000000 | 0.000005 | 0.005714 |
| Sphingobacteriia; Sphingobacteriales; | | | | | | | | | |
| Sphingobacteriaceae; Pedobacter; Other | | | | | | | | | |
| Bacteria; Firmicutes; Clostridia; | 87 | 0.003199 | 1.084354 | 0.000940 | 0.003344 | 0.680387 | 0.000867 | 0.007326 | 0.622857 |
| Clostridiales; Clostridiaceael; | | | | | | | | | |
| Clostridium sensu stricto; Other | | | | | | | | | |
| Bacteria; Bacteroidetes; | 88 | 0.003193 | 0.519496 | 0.051954 | 0.062313 | 0.961259 | 0.100008 | 0.102665 | 0.973333 |
| Bacteroidia; Bacteroidales; | | | | | | | | | |
| Bacteroidaceae; Bacteroides; | | | | | | | | | |
| Other | | | | | | | | | |
| Bacteria; Firmicutes; Bacilli; | 89 | 0.003079 | 0.248198 | 0.000070 | 0.000623 | 0.261501 | 0.000283 | 0.002256 | 0.629524 |
| Lactobacillales; Other; Other; | | | | | | | | | |
| Other | | | | | | | | | |
| Bacteria; Bacteroidetes; | 90 | 0.003069 | 1.250833 | 0.006299 | 0.011554 | 0.728814 | 0.005036 | 0.014266 | 0.706667 |
| Bacteroidia; Bacteroidales; | | | | | | | | | |
| Tannerellaceae; Parabacteroides; | | | | | | | | | |
| Parabacteroides merdae | | | | | | | | | |
| Bacteria; Firmicutes; Clostridia; | 91 | 0.003060 | 2.006992 | 0.005294 | 0.008906 | 0.818402 | 0.002638 | 0.005359 | 0.748571 |
| Clostridiales; Lachnospiraceae; | | | | | | | | | |
| Roseburia; Roseburia inulinivorans | | | | | | | | | |
| Bacteria; Bacteroidetes; | 92 | 0.002978 | 1.208398 | 0.006580 | 0.020688 | 0.859564 | 0.005445 | 0.018848 | 0.809524 |
| Bacteroidia; Bacteroidales; | | | | | | | | | |
| Rikenellaceae; Alistipes; Other | | | | | | | | | |
| Bacteria; Actinobacteria; | 93 | 0.002978 | 0.564124 | 0.001018 | 0.003735 | 0.690073 | 0.001805 | 0.010314 | 0.364762 |
| Actinobacteria; Bifidobacteriales; | | | | | | | | | |
| Bifidobacteriaceae; Bifidobacterium; | | | | | | | | | |
| Bifidobacterium longum | | | | | | | | | |
| Bacteria; Proteobacteria; | 94 | 0.002941 | NA - Div | 0.000002 | 0.000009 | 0.077482 | 0.000000 | 0.000000 | 0.000000 |
| Betaproteobacteria; Burkholderiales; | | | by 0 | | | | | | |
| Burkholderiaceae; Burkholderia; | | | | | | | | | |
| Burkholderia thailandensis | | | | | | | | | |
| Bacteria; Firmicutes; Clostridia; | 95 | 0.002938 | 1.199496 | 0.007517 | 0.010821 | 0.871671 | 0.006267 | 0.010773 | 0.831429 |
| Clostridiales; Lachnospiraceae; | | | | | | | | | |
| Fusicatenibacter; Fusicatenibacter | | | | | | | | | |
| saccharivorans | | | | | | | | | |
| Bacteria; Bacteroidetes; Bacteroidia; | 96 | 0.002910 | 0.367389 | 0.021450 | 0.045829 | 0.927361 | 0.058385 | 0.090300 | 0.932381 |
| Bacteroidales; Bacteroidaceae; | | | | | | | | | |
| Bacteroides; Bacteroides vulgatus | | | | | | | | | |
| Bacteria; Firmicutes; Clostridia; | 97 | 0.002902 | 1.293624 | 0.003396 | 0.005044 | 0.891041 | 0.002625 | 0.008890 | 0.859048 |
| Clostridiales; Lachnospiraceae; | | | | | | | | | |
| Ruminococcus2; Other | | | | | | | | | |
| Bacteria; Firmicutes; Clostridia; | 98 | 0.002888 | 1.481964 | 0.019249 | 0.028624 | 0.905569 | 0.012989 | 0.022303 | 0.909524 |
| Clostridiales; Lachnospiraceae; | | | | | | | | | |
| Anaerostipes; Anaerostipes hadrus | | | | | | | | | |
| Bacteria; Firmicutes; Erysipelotrichia; | 99 | 0.002886 | 5.893999 | 0.000738 | 0.003186 | 0.411622 | 0.000125 | 0.000672 | 0.412381 |
| Erysipelotrichales; Erysipelotrichaceae; | | | | | | | | | |
| Turicibacter; Other | | | | | | | | | |
| Bacteria; Bacteroidetes; | 100 | 0.002850 | 1.820438 | 0.001645 | 0.006094 | 0.496368 | 0.000904 | 0.003470 | 0.234286 |
| Bacteroidia; Bacteroidales; | | | | | | | | | |
| Bacteroidaceae; Bacteroides; | | | | | | | | | |
| Bacteroides finegoldii | | | | | | | | | |

IBS = Irritable Bowel Syndrome;
IBD = Inflammatory Bowel Disease;
RFFR = Random Forest Feature Rank;
# = Rank;
MDA = Mean Decrease Accuracy;
FC = Fold Change;
M = Mean;
SD = Standard Deviation;
DP = Detection Prevalence;

TABLE 12
IBD vs CDI delineation features - (taxonomy)

| Feature | | | | | | | | | |
|---|---|---|---|---|---|---|---|---|---|
| (Taxonomic order: Kingdom; | | | FC | Abundance | | | Abundance | | |
| Phylum; Class; Order; | RFFR | | IBD vs | (IBD) | | | (CDI) | | |
| Family; Genus; Species) | # | MDA | CDI | M | SD | DP | M | SD | DP |
| Bacteria; Firmicutes; Clostridia; | 1 | 0.048326 | 0.003473 | 0.000070 | 0.000989 | 0.020952 | 0.020265 | 0.056449 | 0.764317 |
| Clostridiales; Peptostreptococcaceae; | | | | | | | | | |
| Clostridioides; Clostridioides difficile | | | | | | | | | |
| Bacteria; Firmicutes; Clostridia; | 2 | 0.024571 | 6.152085 | 0.012989 | 0.022303 | 0.909524 | 0.002111 | 0.008309 | 0.275330 |
| Clostridiales; Lachnospiraceae; | | | | | | | | | |
| Anaerostipes; Anaerostipes hadrus | | | | | | | | | |
| Bacteria; Firmicutes; Clostridia; | 3 | 0.023410 | 11.102456 | 0.004953 | 0.010473 | 0.883810 | 0.000446 | 0.002177 | 0.189427 |
| Clostridiales; Lachnospiraceae; | | | | | | | | | |
| Dorea; Other | | | | | | | | | |
| Bacteria; Firmicutes; Clostridia; | 4 | 0.022965 | 0.013233 | 0.000009 | 0.000129 | 0.039048 | 0.000667 | 0.003204 | 0.458150 |
| Clostridiales; Peptostreptococcaceae; | | | | | | | | | |
| Clostridium XI; Other | | | | | | | | | |
| Bacteria; Firmicutes; Clostridia; | 5 | 0.020612 | 9.006374 | 0.006858 | 0.011372 | 0.860000 | 0.000761 | 0.004308 | 0.162996 |
| Clostridiales; Lachnospiraceae; | | | | | | | | | |
| Dorea; Dorea longicatena | | | | | | | | | |
| Bacteria; Firmicutes; Clostridia; | 6 | 0.019235 | 21.367383 | 0.002538 | 0.005560 | 0.817143 | 0.000119 | 0.000698 | 0.110132 |
| Clostridiales; Lachnospiraceae; | | | | | | | | | |
| Coprococcus; Other | | | | | | | | | |
| Bacteria; Proteobacteria; | 7 | 0.018423 | 0.000920 | 0.000002 | 0.000018 | 0.027619 | 0.002035 | 0.008768 | 0.431718 |
| Gammaproteobacteria; Enterobacterales; | | | | | | | | | |
| Enterobacteriaceae; Citrobacter; | | | | | | | | | |
| Other | | | | | | | | | |
| Bacteria; Firmicutes; Clostridia; | 8 | 0.018323 | 4.115199 | 0.024936 | 0.047896 | 0.984762 | 0.006060 | 0.013562 | 0.709251 |
| Clostridiales; Lachnospiraceae; | | | | | | | | | |
| Blautia; Other | | | | | | | | | |
| Bacteria; Firmicutes; Clostridia; | 9 | 0.017080 | 4.603632 | 0.004039 | 0.011111 | 0.932381 | 0.000877 | 0.003080 | 0.431718 |
| Clostridiales; Lachnospiraceae; | | | | | | | | | |
| Anaerostipes; Other | | | | | | | | | |
| Bacteria; Firmicutes; Clostridia; | 10 | 0.016437 | 4.129236 | 0.006417 | 0.015065 | 0.883810 | 0.001554 | 0.009333 | 0.266520 |
| Clostridiales; Lachnospiraceae; | | | | | | | | | |
| Blautia; Blautia faecis | | | | | | | | | |
| Bacteria; Firmicutes; Clostridia; | 11 | 0.016028 | 2.786414 | 0.012087 | 0.020488 | 0.880952 | 0.004338 | 0.022200 | 0.301762 |
| Clostridiales; Lachnospiraceae; | | | | | | | | | |
| Blautia; Blautia luti | | | | | | | | | |
| Bacteria; Firmicutes; Clostridia; | 12 | 0.014830 | 3.568142 | 0.058907 | 0.051085 | 0.991429 | 0.016509 | 0.039130 | 0.850220 |
| Clostridiales; Lachnospiraceae; | | | | | | | | | |
| Other; Other | | | | | | | | | |
| Bacteria; Proteobacteria; | 13 | 0.014519 | 0.085127 | 0.012114 | 0.053094 | 0.710476 | 0.142299 | 0.194372 | 0.907489 |
| Gammaproteobacteria; Enterobacterales; | | | | | | | | | |
| Enterobacteriaceae; Other; Other | | | | | | | | | |
| Bacteria; Firmicutes; Clostridia; | 14 | 0.014291 | 9.080515 | 0.005227 | 0.008361 | 0.887619 | 0.000576 | 0.002224 | 0.306167 |
| Clostridiales; Lachnospiraceae; | | | | | | | | | |
| Roseburia; Other | | | | | | | | | |
| Bacteria; Firmicutes; Clostridia; | 15 | 0.013755 | 8.938892 | 0.006065 | 0.014684 | 0.829524 | 0.000678 | 0.003388 | 0.213656 |
| Clostridiales; Lachnospiraceae; | | | | | | | | | |
| Blautia; Blautia obeum | | | | | | | | | |
| Bacteria; Firmicutes; Clostridia; | 16 | 0.012960 | 12.431066 | 0.004266 | 0.007594 | 0.781905 | 0.000343 | 0.002312 | 0.129956 |
| Clostridiales; Lachnospiraceae; | | | | | | | | | |
| Coprococcus; Coprococcus comes | | | | | | | | | |
| Bacteria; Firmicutes; Negativicutes; | 17 | 0.012150 | 0.085546 | 0.000651 | 0.015259 | 0.138095 | 0.007608 | 0.034840 | 0.504405 |
| Veillonellales; Veillonellaceae; | | | | | | | | | |
| Veillonella; Veillonella parvula | | | | | | | | | |
| Bacteria; Firmicutes; Other; | 18 | 0.012115 | 11.203883 | 0.001770 | 0.003186 | 0.906667 | 0.000158 | 0.000446 | 0.422907 |
| Other; Other; Other; Other | | | | | | | | | |
| Bacteria; Firmicutes; Clostridia; | 19 | 0.011966 | 3.444316 | 0.013696 | 0.018995 | 0.987619 | 0.003976 | 0.012514 | 0.726872 |
| Clostridiales; Other; Other; Other | | | | | | | | | |
| Bacteria; Firmicutes; Bacilli; | 20 | 0.011536 | 0.024886 | 0.001218 | 0.009072 | 0.381905 | 0.048932 | 0.144190 | 0.775330 |
| Lactobacillales; Enterococcaceae; | | | | | | | | | |
| Enterococcus; Other | | | | | | | | | |
| Bacteria; Firmicutes; Bacilli; | 21 | 0.009876 | 0.022380 | 0.000032 | 0.000422 | 0.064762 | 0.001415 | 0.006020 | 0.392070 |
| Lactobacillales; Enterococcaceae; | | | | | | | | | |
| Other; Other | | | | | | | | | |
| Bacteria; Firmicutes; Clostridia; | 22 | 0.009449 | 7.242833 | 0.003542 | 0.008937 | 0.771429 | 0.000489 | 0.002122 | 0.213656 |
| Clostridiales; Lachnospiraceae; | | | | | | | | | |
| Roseburia; Roseburia intestinalis | | | | | | | | | |
| Bacteria; Firmicutes; Clostridia; | 23 | 0.009315 | 2.723538 | 0.014041 | 0.022190 | 0.978095 | 0.005156 | 0.013758 | 0.632159 |
| Clostridiales; Lachnospiraceae; | | | | | | | | | |
| Lachnospiracea_incertae_sedis; | | | | | | | | | |
| Other | | | | | | | | | |
| Bacteria; Other; Other; Other; | 24 | 0.008784 | 32.493142 | 0.020907 | 0.037823 | 0.939048 | 0.000643 | 0.004047 | 0.594714 |
| Other; Other; Other | | | | | | | | | |
| Bacteria; Firmicutes; Bacilli; | 25 | 0.008722 | 5.827834 | 0.002219 | 0.004944 | 0.642857 | 0.000381 | 0.002913 | 0.101322 |
| Lactobacillales; Lactobacillaceae; | | | | | | | | | |
| Lactobacillus; Lactobacillus | | | | | | | | | |
| rogosae | | | | | | | | | |
| Bacteria; Firmicutes; Clostridia; | 26 | 0.007865 | 2.733512 | 0.006267 | 0.010773 | 0.831429 | 0.002293 | 0.012148 | 0.312775 |
| Clostridiales; Lachnospiraceae; | | | | | | | | | |
| Fusicatenibacter; | | | | | | | | | |
| Fusicatenibacter saccharivorans | | | | | | | | | |
| Bacteria; Firmicutes; Clostridia; | 27 | 0.007784 | 2.111927 | 0.001449 | 0.002690 | 0.775238 | 0.000686 | 0.003778 | 0.220264 |
| Clostridiales; Lachnospiraceae; | | | | | | | | | |
| Fusicatenibacter; Other | | | | | | | | | |
| Bacteria; Firmicutes; Clostridia; | 28 | 0.007348 | 9.273969 | 0.004146 | 0.008590 | 0.703810 | 0.000447 | 0.002306 | 0.145374 |
| Clostridiales; Eubacteriaceae; | | | | | | | | | |
| Eubacterium; [Eubacterium] | | | | | | | | | |
| eligens | | | | | | | | | |
| Bacteria; Actinobacteria; | 29 | 0.007221 | 3.652602 | 0.007053 | 0.023275 | 0.728571 | 0.001931 | 0.012523 | 0.248899 |
| Coriobacteriia; Coriobacteriales; | | | | | | | | | |
| Coriobacteriaceae; Collinsella; | | | | | | | | | |
| Collinsella aerofaciens | | | | | | | | | |
| Bacteria; Firmicutes; Clostridia; | 30 | 0.006858 | 2.227315 | 0.040297 | 0.054719 | 0.911429 | 0.018092 | 0.049472 | 0.491189 |
| Clostridiales; Ruminococcaceae; | | | | | | | | | |
| Faecalibacterium; Other | | | | | | | | | |
| Bacteria; Firmicutes; Clostridia; | 31 | 0.006769 | 3.289436 | 0.002638 | 0.005359 | 0.748571 | 0.000802 | 0.004536 | 0.198238 |
| Clostridiales; Lachnospiraceae; | | | | | | | | | |
| Roseburia; Roseburia inulinivorans | | | | | | | | | |
| Bacteria; Firmicutes; | 32 | 0.006751 | 0.087693 | 0.000444 | 0.003028 | 0.646667 | 0.005062 | 0.019598 | 0.665198 |
| Erysipelotrichia; | | | | | | | | | |
| Erysipelotrichales; | | | | | | | | | |
| Erysipelotrichaceae; | | | | | | | | | |
| Erysipelatoclostridium; | | | | | | | | | |
| [Clostridium] innocuum | | | | | | | | | |
| Bacteria; Bacteroidetes; | 33 | 0.006385 | 2.413506 | 0.005370 | 0.009744 | 0.720952 | 0.002225 | 0.009918 | 0.209251 |
| Bacteroidia; Bacteroidales; | | | | | | | | | |
| Rikenellaceae; Alistipes; | | | | | | | | | |
| Alistipes putredinis | | | | | | | | | |
| Bacteria; Bacteroidetes; | 34 | 0.006272 | 17.358566 | 0.002447 | 0.004365 | 0.805714 | 0.000141 | 0.000320 | 0.341410 |
| Bacteroidia; Bacteroidales; | | | | | | | | | |
| Other; Other; Other | | | | | | | | | |
| Bacteria; Firmicutes; Clostridia; | 35 | 0.006201 | 68.531770 | 0.001859 | 0.007589 | 0.577143 | 0.000027 | 0.000160 | 0.085903 |
| Clostridiales; Peptostreptococcaceae; | | | | | | | | | |
| Romboutsia; Other | | | | | | | | | |
| Bacteria; Verrucomicrobia; | 36 | 0.005835 | 0.216159 | 0.004967 | 0.025429 | 0.47690 | 0.022979 | 0.087965 | 0.546256 |
| Verrucomicrobiae; | | | | | | | | | |
| Verrucomicrobiales; | | | | | | | | | |
| Akkermansiaceae; Akkermansia; | | | | | | | | | |
| Akkermansia muciniphila | | | | | | | | | |
| Bacteria; Firmicutes; Clostridia; | 37 | 0.005802 | 4.534829 | 0.023730 | 0.048235 | 0.938095 | 0.005233 | 0.027016 | 0.561674 |
| Clostridiales; Ruminococcaceae; | | | | | | | | | |
| Other; Other | | | | | | | | | |
| Bacteria; Actinobacteria; | 38 | 0.005564 | 5.121888 | 0.003859 | 0.011240 | 0.681905 | 0.000753 | 0.004871 | 0.196035 |
| Actinobacteria; Coriobacteriales; | | | | | | | | | |
| Coriobacteriaceae; Collinsella; | | | | | | | | | |
| Other | | | | | | | | | |
| Bacteria; Firmicutes; Clostridia; | 39 | 0.005508 | 1.912528 | 0.001088 | 0.003036 | 0.729524 | 0.000569 | 0.006346 | 0.213656 |
| Clostridiales; Lachnospiraceae; | | | | | | | | | |
| Roseburia; Roseburia hominis | | | | | | | | | |
| Bacteria; Proteobacteria; | 40 | 0.005506 | 0.013041 | 0.000001 | 0.000006 | 0.030476 | 0.000061 | 0.000200 | 0.193833 |
| Gammaproteobacteria; Enterobacterales; | | | | | | | | | |
| Pectobacteriaceae; Pectobacterium; | | | | | | | | | |
| Pectobacterium carotovorum | | | | | | | | | |
| Bacteria; Proteobacteria; | 41 | 0.005426 | 0.152818 | 0.004755 | 0.018306 | 0.795238 | 0.031113 | 0.060092 | 0.856828 |
| Gammaproteobacteria; Enterobacteriales; | | | | | | | | | |
| Enterobacteriaceae; Other; Other | | | | | | | | | |
| Bacteria; Firmicutes; Negativicutes; | 42 | 0.005414 | 54.804655 | 0.001457 | 0.006792 | 0.551429 | 0.000027 | 0.000283 | 0.035242 |
| Selenomonadales; Acidaminococcaceae; | | | | | | | | | |
| Acidaminococcus; Other | | | | | | | | | |
| Bacteria; Bacteroidetes; Bacteroidia; | 43 | 0.005273 | 3.411011 | 0.003311 | 0.008964 | 0.650476 | 0.000971 | 0.006250 | 0.101322 |
| Bacteroidales; Porphyromonadaceae; | | | | | | | | | |
| Barnesiella; Other | | | | | | | | | |
| Bacteria; Bacteroidetes; Bacteroidia; | 44 | 0.005262 | 8.717579 | 0.028032 | 0.087686 | 0.770476 | 0.003216 | 0.027796 | 0.297357 |
| Bacteroidales; Prevotellaceae; | | | | | | | | | |
| Prevotella; Other | | | | | | | | | |
| Bacteria; Bacteroidetes; Other; | 45 | 0.005006 | 69.490224 | 0.001610 | 0.004345 | 0.674286 | 0.000023 | 0.000069 | 0.209251 |
| Other; Other; Other; Other | | | | | | | | | |
| Bacteria; Bacteroidetes; Bacteroidia; | 46 | 0.004916 | 0.084182 | 0.000920 | 0.003588 | 0.313333 | 0.010934 | 0.036540 | 0.528634 |
| Bacteroidales; Bacteroidaceae; | | | | | | | | | |
| Bacteroides; Bacteroides | | | | | | | | | |
| xylanisolvens | | | | | | | | | |
| Bacteria; Firmicutes; Bacilli; | 47 | 0.004874 | 0.020231 | 0.000003 | 0.000033 | 0.030476 | 0.000148 | 0.000493 | 0.312775 |
| Lactobacillales; Enterococcaceae; | | | | | | | | | |
| Enterococcus; Enterococcus | | | | | | | | | |
| saccharolyticus | | | | | | | | | |
| Bacteria; Firmicutes; Clostridia; | 48 | 0.004689 | 0.037033 | 0.000064 | 0.000752 | 0.067619 | 0.001720 | 0.007262 | 0.385463 |
| Clostridiales; Clostridiaceae; | | | | | | | | | |
| Clostridium; Clostridium | | | | | | | | | |
| paraputrificum | | | | | | | | | |
| Bacteria; Bacteroidetes; | 49 | 0.004620 | 8.328950 | 0.028580 | 0.096181 | 0.687619 | 0.003431 | 0.031801 | 0.176211 |
| Bacteroidia; Bacteroidales; | | | | | | | | | |
| Prevotellaceae; Prevotella; | | | | | | | | | |
| Prevotella copri | | | | | | | | | |
| Bacteria; Actinobacteria; | 50 | 0.004186 | 6.119334 | 0.000346 | 0.002140 | 0.632381 | 0.000056 | 0.000234 | 0.121145 |
| Actinobacteria; Coriobacteriales; | | | | | | | | | |
| Coriobacteriaceae; Other; Other | | | | | | | | | |
| Bacteria; Firmicutes; Clostridia; | 51 | 0.004170 | 10.828662 | 0.004272 | 0.012950 | 0.694286 | 0.000395 | 0.002295 | 0.204846 |
| Clostridiales; Ruminococcaceae; | | | | | | | | | |
| Ruminococcus; Other | | | | | | | | | |
| Bacteria; Bacteroidetes; | 52 | 0.004157 | 21.255918 | 0.001190 | 0.003569 | 0.533333 | 0.000056 | 0.000396 | 0.059471 |
| Bacteroidia; Bacteroidales; | | | | | | | | | |
| Rikenellaceae; Alistipes; | | | | | | | | | |
| Alistipes obesi | | | | | | | | | |
| Bacteria; Firmicutes; Clostridia; | 53 | 0.004072 | 1.446276 | 0.003104 | 0.009542 | 0.689524 | 0.002146 | 0.026081 | 0.191630 |
| Clostridiales; Ruminococcaceae; | | | | | | | | | |
| Ruminococcus; Ruminococcus faecis | | | | | | | | | |
| Bacteria; Proteobacteria; | 54 | 0.004048 | 170.525637 | 0.001366 | 0.008723 | 0.554286 | 0.000008 | 0.000040 | 0.070485 |
| Betaproteobacteria; Burkholderiales; | | | | | | | | | |
| Other; Other; Other | | | | | | | | | |
| Bacteria; Firmicutes; Clostridia; | 55 | 0.004000 | 9.637715 | 0.006795 | 0.019336 | 0.617143 | 0.000705 | 0.004419 | 0.125551 |
| Clostridiales; Ruminococcaceae; | | | | | | | | | |
| Ruminococcus; Ruminococcus bromii | | | | | | | | | |
| Bacteria; Actinobacteria; | 56 | 0.003957 | 1.864602 | 0.022880 | 0.052680 | 0.916190 | 0.012271 | 0.041291 | 0.555066 |
| Actinobacteria; Bifidobacteriales; | | | | | | | | | |
| Bifidobacteriaceae; Bifidobacterium; Other | | | | | | | | | |
| Bacteria; Proteobacteria; | 57 | 0.003954 | 0.008811 | 0.000004 | 0.000080 | 0.008571 | 0.000454 | 0.005274 | 0.224670 |
| Gammaproteobacteria; Enterobacterales; | | | | | | | | | |
| Enterobacteriaceae; Salmonella; | | | | | | | | | |
| Salmonella enterica | | | | | | | | | |
| Bacteria; Firmicutes; Clostridia; | 58 | 0.003850 | 7.944086 | 0.001136 | 0.006502 | 0.476190 | 0.000143 | 0.002151 | 0.037445 |
| Clostridiales; Lachnospiraceae; | | | | | | | | | |
| Lachnoclostridium; Other | | | | | | | | | |
| Bacteria; Bacteroidetes; Bacteroidia; | 59 | 0.003838 | 0.447019 | 0.000895 | 0.001940 | 0.653333 | 0.002001 | 0.009758 | 0.178414 |
| Bacteroidales; Odoribacteraceae; | | | | | | | | | |
| Odoribacter; Odoribacter splanchnicus | | | | | | | | | |
| Bacteria; Firmicutes; Clostridia; | 60 | 0.003792 | 1.625713 | 0.005409 | 0.016380 | 0.690476 | 0.003327 | 0.019940 | 0.207048 |
| Clostridiales; Ruminococcaceae; | | | | | | | | | |
| Gemmiger; Gemmiger formicilis | | | | | | | | | |
| Bacteria; Actinobacteria; | 61 | 0.003631 | 9.964229 | 0.011395 | 0.038469 | 0.388571 | 0.001144 | 0.007492 | 0.244493 |
| Actinobacteria; Bifidobacteriales; | | | | | | | | | |
| Bifidobacteriaceae; Bifidobacterium; | | | | | | | | | |
| Bifidobacterium adolescentis | | | | | | | | | |
| Bacteria; Firmicutes; Clostridia; | 62 | 0.003623 | 0.268233 | 0.002905 | 0.011580 | 0.394286 | 0.010829 | 0.029193 | 0.711454 |
| Clostridiales; Lachnospiraceae; | | | | | | | | | |
| Lachnoclostridium; [Clostridium] | | | | | | | | | |
| bolteae | | | | | | | | | |
| Bacteria; Firmicutes; Erysipelotrichia; | 63 | 0.003467 | 0.194184 | 0.001401 | 0.006731 | 0.540952 | 0.007217 | 0.022214 | 0.658590 |
| Erysipelotrichales; Erysipelotrichaceae; | | | | | | | | | |
| Erysipelatoclostridium; | | | | | | | | | |
| Erysipelatoclostridium ramosum | | | | | | | | | |
| Bacteria; Proteobacteria; | 64 | 0.003411 | 3.992119 | 0.001540 | 0.007220 | 0.550476 | 0.000386 | 0.003110 | 0.143172 |
| Betaproteobacteria; Burkholderiales; | | | | | | | | | |
| Sutterellaceae; Sutterella; Other | | | | | | | | | |
| Bacteria; Bacteroidetes; | 65 | 0.003327 | 1.160602 | 0.005445 | 0.018848 | 0.809524 | 0.004691 | 0.027357 | 0.407489 |
| Bacteroidia; Bacteroidales; | | | | | | | | | |
| Rikenellaceae; Alistipes; Other | | | | | | | | | |
| Bacteria; Firmicutes; Clostridia; | 66 | 0.003255 | 0.101808 | 0.000140 | 0.001056 | 0.422857 | 0.001372 | 0.005976 | 0.385463 |
| Clostridiales; Clostridiaceae; | | | | | | | | | |
| Hungatella; Hungatella effluvii | | | | | | | | | |
| Bacteria; Bacteroidetes; | 67 | 0.003255 | 0.137451 | 0.000139 | 0.000922 | 0.166667 | 0.001014 | 0.005458 | 0.352423 |
| Bacteroidia; Bacteroidales; | | | | | | | | | |
| Bacteroidaceae; Bacteroides; | | | | | | | | | |
| Bacteroides koreensis | | | | | | | | | |
| Bacteria; Verrucomicrobia; | 68 | 0.003231 | 0.239652 | 0.004987 | 0.032519 | 0.579048 | 0.020811 | 0.074687 | 0.500000 |
| Verrucomicrobiae; Verrucomicrobiales; | | | | | | | | | |
| Verrucomicrobiaceae; Akkermansia; | | | | | | | | | |
| Other | | | | | | | | | |
| Bacteria; Firmicutes; Clostridia; | 69 | 0.003080 | 2.393841 | 0.004586 | 0.025790 | 0.334286 | 0.001916 | 0.009484 | 0.396476 |
| Clostridiales; Lachnospiraceae; | | | | | | | | | |
| Blautia; Blautia hominis | | | | | | | | | |
| Bacteria; Firmicutes; Bacilli; | 70 | 0.002959 | 0.062031 | 0.000283 | 0.002256 | 0.629524 | 0.004568 | 0.016546 | 0.590308 |
| Lactobacillales; Other; Other; | | | | | | | | | |
| Other | | | | | | | | | |
| Bacteria; Firmicutes; Clostridia; | 71 | 0.002950 | 0.067966 | 0.000258 | 0.003641 | 0.151429 | 0.003790 | 0.020609 | 0.418502 |
| Clostridiales; Lachnospiraceae; | | | | | | | | | |
| Blautia; Blautia producta | | | | | | | | | |
| Bacteria; Proteobacteria; | 72 | 0.002842 | 0.000000 | 0.000000 | 0.000000 | 0.000000 | 0.000053 | 0.000227 | 0.160793 |
| Gammaproteobacteria; Enterobacterales; | | | | | | | | | |
| Enterobacteriaceae; Klebsiella; | | | | | | | | | |
| Klebsiella quasipneumoniae | | | | | | | | | |
| Bacteria; Proteobacteria; | 73 | 0.002787 | 1.652168 | 0.001624 | 0.006916 | 0.468571 | 0.000983 | 0.012756 | 0.061674 |
| Betaproteobacteria; Burkholderiales; | | | | | | | | | |
| Sutterellaceae; Sutterella; | | | | | | | | | |
| Sutterella wadsworthensis | | | | | | | | | |
| Bacteria; Firmicutes; Clostridia; | 74 | 0.002715 | 1.072168 | 0.000363 | 0.001304 | 0.645714 | 0.000338 | 0.002766 | 0.273128 |
| Clostridiales; Peptostreptococcaceae; | | | | | | | | | |
| Intestinibacter; Other | | | | | | | | | |
| Bacteria; Firmicutes; | 75 | 0.002702 | 0.261395 | 0.000049 | 0.000426 | 0.360000 | 0.000186 | 0.001803 | 0.068282 |
| Erysipelotrichia; Erysipelotrichales; | | | | | | | | | |
| Erysipelotrichaceae; Solobacterium; | | | | | | | | | |
| Solobacterium moorei | | | | | | | | | |
| Bacteria; Firmicutes; Clostridia; | 76 | 0.002694 | 0.169285 | 0.000797 | 0.002761 | 0.777143 | 0.004709 | 0.015516 | 0.585903 |
| Clostridiales; Ruminococcaceae; | | | | | | | | | |
| Ruthenibacterium; Ruthenibacterium | | | | | | | | | |
| lactatiformans | | | | | | | | | |
| Bacteria; Firmicutes; | 77 | 0.002692 | 0.264509 | 0.000605 | 0.004367 | 0.636190 | 0.002288 | 0.009327 | 0.581498 |
| Erysipelotrichia; Erysipelotrichales; | | | | | | | | | |
| Erysipelotrichaceae; Clostridium | | | | | | | | | |
| XVIII; Other | | | | | | | | | |
| Bacteria; Firmicutes; Clostridia; | 78 | 0.002638 | 0.361490 | 0.001102 | 0.003027 | 0.803810 | 0.003049 | 0.014367 | 0.709251 |
| Clostridiales; Peptostreptococcaceae; | | | | | | | | | |
| Other; Other | | | | | | | | | |
| Bacteria; Actinobacteria; | 79 | 0.002627 | 2.520416 | 0.001805 | 0.010314 | 0.364762 | 0.000716 | 0.003769 | 0.240088 |
| Actinobacteria; Bifidobacteriales; | | | | | | | | | |
| Bifidobacteriaceae; Bifidobacterium; | | | | | | | | | |
| Bifidobacterium longum | | | | | | | | | |
| Bacteria; Actinobacteria; | 80 | 0.002581 | 46.237711 | 0.000034 | 0.000091 | 0.425714 | 0.000001 | 0.000009 | 0.011013 |
| Actinobacteria; Actinomycetales; | | | | | | | | | |
| Actinomycetaceae; Other; Other | | | | | | | | | |
| Bacteria; Firmicutes; Clostridia; | 81 | 0.002556 | 2.307456 | 0.000864 | 0.003153 | 0.328571 | 0.000374 | 0.002447 | 0.145374 |
| Clostridiales; Ruminococcaceae; | | | | | | | | | |
| Agathobaculum; Agathobaculum | | | | | | | | | |
| butyriciproducens | | | | | | | | | |
| Bacteria; Firmicutes; Clostridia; | 82 | 0.002550 | 0.608632 | 0.008229 | 0.028839 | 0.841905 | 0.013521 | 0.029521 | 0.647577 |
| Clostridiales; Lachnospiraceae; | | | | | | | | | |
| Blautia; [Ruminococcus] | | | | | | | | | |
| gnavus | | | | | | | | | |
| Bacteria; Firmicutes; Clostridia; | 83 | 0.002513 | 1.803142 | 0.025044 | 0.061261 | 0.471429 | 0.013889 | 0.039999 | 0.533040 |
| Clostridiales; Lachnospiraceae; | | | | | | | | | |
| Blautia; Blautia wexlerae | | | | | | | | | |
| Bacteria; Firmicutes; Clostridia; | 84 | 0.002482 | 29.084736 | 0.000350 | 0.000968 | 0.463810 | 0.000012 | 0.000126 | 0.037445 |
| Clostridiales; Ruminococcaceae; | | | | | | | | | |
| Intestinimonas; Other | | | | | | | | | |
| Bacteria; Firmicutes; Clostridia; | 85 | 0.002462 | 1.391015 | 0.004213 | 0.017069 | 0.440000 | 0.003029 | 0.010903 | 0.334802 |
| Clostridiales; Lachnospiraceae; | | | | | | | | | |
| Faecalimonas; Faecalimonas | | | | | | | | | |
| umbilicata | | | | | | | | | |
| Bacteria; Firmicutes; Clostridia; | 86 | 0.002408 | 0.379372 | 0.002625 | 0.008890 | 0.859048 | 0.006920 | 0.021979 | 0.466960 |
| Clostridiales; Lachnospiraceae; | | | | | | | | | |
| Ruminococcus2; Other | | | | | | | | | |
| Bacteria; Proteobacteria; | 87 | 0.002378 | 0.227057 | 0.000021 | 0.000214 | 0.102857 | 0.000092 | 0.000328 | 0.262115 |
| Gammaproteobacteria; Enterobacterales; | | | | | | | | | |
| Enterobacteriaceae; Shigella; | | | | | | | | | |
| Shigella dysenteriae | | | | | | | | | |
| Bacteria; Bacteroidetes; | 88 | 0.002364 | 2.259560 | 0.058385 | 0.090300 | 0.932381 | 0.025839 | 0.077907 | 0.674009 |
| Bacteroidia; Bacteroidales; | | | | | | | | | |
| Bacteroidaceae; Bacteroides; | | | | | | | | | |
| Bacteroides vulgatus | | | | | | | | | |
| Bacteria; Bacteroidetes; | 89 | 0.002353 | 8.917954 | 0.003676 | 0.016371 | 0.485714 | 0.000412 | 0.003673 | 0.077093 |
| Bacteroidia; Bacteroidales; | | | | | | | | | |
| Bacteroidaceae; Bacteroides; | | | | | | | | | |
| Bacteroides eggerthii | | | | | | | | | |
| Bacteria; Firmicutes; Clostridia; | 90 | 0.002348 | 0.309577 | 0.001812 | 0.004738 | 0.907619 | 0.005855 | 0.013353 | 0.786344 |
| Clostridiales; Lachnospiraceae; | | | | | | | | | |
| Clostridium XIVa; Other | | | | | | | | | |
| Bacteria; Actinobacteria; | 91 | 0.002301 | 0.397177 | 0.000586 | 0.002024 | 0.633333 | 0.001476 | 0.005130 | 0.486784 |
| Coriobacteriia; Eggerthellales; | | | | | | | | | |
| Eggerthellaceae; Eggerthella; | | | | | | | | | |
| Eggerthella lenta | | | | | | | | | |
| Bacteria; Bacteroidetes; | 92 | 0.002298 | 2.850960 | 0.001917 | 0.006693 | 0.488571 | 0.000672 | 0.004898 | 0.072687 |
| Bacteroidia; Bacteroidales; | | | | | | | | | |
| Prevotellaceae; Paraprevotella; | | | | | | | | | |
| Other | | | | | | | | | |
| Bacteria; Firmicutes; Clostridia; | 93 | 0.002296 | 10.604531 | 0.000034 | 0.000078 | 0.503810 | 0.000003 | 0.000020 | 0.048458 |
| Other; Other; Other; Other | | | | | | | | | |
| Bacteria; Firmicutes; Negativicutes; | 94 | 0.002252 | 0.180724 | 0.002534 | 0.019512 | 0.676190 | 0.014022 | 0.044571 | 0.616740 |
| Veillonellales; Veillonellaceae; | | | | | | | | | |
| Veillonella; Veillonella dispar | | | | | | | | | |
| Bacteria; Actinobacteria; | 95 | 0.002249 | 0.188958 | 0.000082 | 0.000259 | 0.501905 | 0.000435 | 0.001791 | 0.281938 |
| Actinobacteria; Coriobacteriales; | | | | | | | | | |
| Coriobacteriaceae; Eggerthella; | | | | | | | | | |
| Other | | | | | | | | | |
| Bacteria; Firmicutes; Bacilli; | 96 | 0.002174 | 0.204004 | 0.000047 | 0.000654 | 0.142857 | 0.000229 | 0.004154 | 0.026432 |
| Lactobacillales; Lactobacillaceae; | | | | | | | | | |
| Lactobacillus; Lactobacillus sakei | | | | | | | | | |
| Bacteria; Firmicutes; Bacilli; | 97 | 0.002114 | 0.963700 | 0.013303 | 0.034735 | 0.982857 | 0.013804 | 0.038345 | 0.894273 |
| Lactobacillales; Streptococcaceae; | | | | | | | | | |
| Streptococcus; Other | | | | | | | | | |
| Bacteria; Bacteroidetes; Bacteroidia; | 98 | 0.002069 | 1.422532 | 0.100008 | 0.102665 | 0.973333 | 0.070303 | 0.112647 | 0.909692 |
| Bacteroidales; Bacteroidaceae; | | | | | | | | | |
| Bacteroides; Other | | | | | | | | | |
| Bacteria; Firmicutes; Clostridia; | 99 | 0.002069 | 1.113561 | 0.002963 | 0.010174 | 0.800952 | 0.002661 | 0.011396 | 0.466960 |
| Clostridiales; Peptostreptococcaceae; | | | | | | | | | |
| Intestinibacter; Intestinibacter | | | | | | | | | |
| bartlettii | | | | | | | | | |
| Bacteria; Firmicutes; Clostridia; | 100 | 0.002000 | 0.107240 | 0.000019 | 0.000167 | 0.205714 | 0.000181 | 0.000841 | 0.222467 |
| Clostridiales; Lachnospiraceae; | | | | | | | | | |
| Hungatella; Other | | | | | | | | | |

IBD = Inflammatory Bowel Disease;
CDI = Clostridiodes difficile Infection;
RFFR = Random Forest Feature Rank;
# = Rank;
MDA = Mean Decrease Accuracy;
FC = Fold Change;
M = Mean;
SD = Standard Deviation;
DP = Detection Prevalence;

TABLE 13
IBD (Ulcerative Colitis) vs Control delineation features - (taxonomy)

| Feature | | | | | | | | | |
|---|---|---|---|---|---|---|---|---|---|
| (Taxonomic order: Kingdom; | | | FC | | | | | | |
| Phylum; Class; Order; | RFFR | | UC vs | Abundance (Ctrl) | | | Abundance (UC) | | |
| Family; Genus; Species) | # | MDA | Ctrl | M | SD | DP | M | SD | DP |
| Bacteria; Firmicutes; Clostridia; | 1 | 0.014379 | 0.476019 | 0.027343 | 0.033723 | 0.892259 | 0.013016 | 0.029152 | 0.432810 |
| Clostridia; Clostridiales; | | | | | | | | | |
| Lachnospiraceae; Not Available; | | | | | | | | | |
| [Eubacterium] rectale | | | | | | | | | |
| Bacteria; Firmicutes; Clostridia; | 2 | 0.013473 | 80.381153 | 0.000004 | 0.000027 | 0.059623 | 0.000284 | 0.002361 | 0.500873 |
| Clostridiales; Lachnospiraceae; | | | | | | | | | |
| Lachnoclostridium; Other | | | | | | | | | |
| Bacteria; Firmicutes; Clostridia; | 3 | 0.013339 | 0.799618 | 0.026222 | 0.040290 | 0.913180 | 0.020967 | 0.049016 | 0.474695 |
| Clostridiales; Lachnospiraceae; | | | | | | | | | |
| Blautia; Blautia wexlerae | | | | | | | | | |
| Bacteria; Bacteroidetes; | 4 | 0.013137 | 0.122457 | 0.005759 | 0.017531 | 0.798117 | 0.000705 | 0.002697 | 0.345550 |
| Bacteroidia; Bacteroidales; | | | | | | | | | |
| Bacteroidaceae; Bacteroides; | | | | | | | | | |
| Bacteroides xylanisolvens | | | | | | | | | |
| Bacteria; Firmicutes; Clostridia; | 5 | 0.010948 | 16.491037 | 0.000028 | 0.000117 | 0.142259 | 0.000467 | 0.001038 | 0.565445 |
| Clostridiales; Ruminococcaceae; | | | | | | | | | |
| Intestinimonas; Other | | | | | | | | | |
| Bacteria; Bacteroidetes; | 6 | 0.010757 | 6.242436 | 0.000343 | 0.002384 | 0.446653 | 0.002144 | 0.005280 | 0.692845 |
| Other; Other; Other; Other; Other | | | | | | | | | |
| Bacteria; Firmicutes; | 7 | 0.009802 | 5.408171 | 0.000201 | 0.002675 | 0.170502 | 0.001089 | 0.004955 | 0.605585 |
| Negativicutes; Selenomonadales; | | | | | | | | | |
| Acidaminococcaceae; | | | | | | | | | |
| Acidaminococcus; Other | | | | | | | | | |
| Bacteria; Bacteroidetes; | 8 | 0.009746 | 3.001514 | 0.001006 | 0.004950 | 0.701883 | 0.003020 | 0.005204 | 0.842932 |
| Bacteroidia; Bacteroidales; | | | | | | | | | |
| Other; Other; Other | | | | | | | | | |
| Bacteria; Firmicutes; Bacilli; | 9 | 0.008625 | 5.042526 | 0.000034 | 0.000231 | 0.221757 | 0.000169 | 0.002014 | 0.630017 |
| Lactobacillales; Other; Other; Other | | | | | | | | | |
| Bacteria; Firmicutes; Other; Other; | 10 | 0.008104 | 2.427599 | 0.000772 | 0.001531 | 0.731172 | 0.001875 | 0.002546 | 0.942408 |
| Other; Other; Other | | | | | | | | | |
| Bacteria; Actinobacteria; | 11 | 0.008019 | 2.363177 | 0.000030 | 0.000132 | 0.206067 | 0.000071 | 0.000170 | 0.609075 |
| Actinobacteria; Bifidobacteriales; | | | | | | | | | |
| Bifidobacteriaceae; Other; Other | | | | | | | | | |
| Bacteria; Actinobacteria; | 12 | 0.007946 | 6.733162 | 0.000004 | 0.000028 | 0.060669 | 0.000029 | 0.000075 | 0.453752 |
| Actinobacteria; Actinomycetales; | | | | | | | | | |
| Actinomycetaceae; Other; Other | | | | | | | | | |
| Bacteria; Firmicutes; Clostridia; | 13 | 0.007881 | 0.679150 | 0.003574 | 0.006957 | 0.786611 | 0.002427 | 0.006841 | 0.413613 |
| Clostridiales; Lachnospiraceae; | | | | | | | | | |
| Roseburia; Roseburia faecis | | | | | | | | | |
| Bacteria; Other; Other; | 14 | 0.007376 | 4.922276 | 0.005479 | 0.021341 | 0.819038 | 0.026969 | 0.043756 | 0.954625 |
| Other; Other; Other; Other | | | | | | | | | |
| Bacteria; Firmicutes; Clostridia; | 15 | 0.007263 | 0.455811 | 0.002003 | 0.003530 | 0.778243 | 0.000913 | 0.002441 | 0.382199 |
| Clostridiales; Ruminococcaceae; | | | | | | | | | |
| Agathobaculum; | | | | | | | | | |
| Agathobaculum butyriciproducens | | | | | | | | | |
| Bacteria; Verrucomicrobia; | 16 | 0.007047 | 0.380353 | 0.008451 | 0.033406 | 0.643305 | 0.003214 | 0.014232 | 0.603839 |
| Verrucomicrobiae; Verrucomicrobiales; | | | | | | | | | |
| Verrucomicrobiaceae; | | | | | | | | | |
| Akkermansia; Other | | | | | | | | | |
| Bacteria; Firmicutes; Clostridia; | 17 | 0.006962 | 8.265424 | 0.000006 | 0.000030 | 0.071130 | 0.000048 | 0.000116 | 0.431065 |
| Clostridiales; Ruminococcaceae; | | | | | | | | | |
| Subdoligranulum; Other | | | | | | | | | |
| Bacteria; Proteobacteria; | 18 | 0.006642 | 0.191680 | 0.018587 | 0.056891 | 0.816946 | 0.003563 | 0.024830 | 0.661431 |
| Gammaproteobacteria; Enterobacterales; | | | | | | | | | |
| Enterobacteriaceae; Other; Other | | | | | | | | | |
| Bacteria; Firmicutes; Clostridia; | 19 | 0.006630 | 0.684426 | 0.007228 | 0.030628 | 0.802301 | 0.004947 | 0.024219 | 0.439791 |
| Clostridiales; Peptostreptococcaceae; | | | | | | | | | |
| Romboutsia; | | | | | | | | | |
| Romboutsia timonensis | | | | | | | | | |
| Bacteria; Bacteroidetes; Bacteroidia; | 20 | 0.006625 | 0.621235 | 0.026934 | 0.054373 | 0.777197 | 0.016732 | 0.043743 | 0.403141 |
| Bacteroidales; Bacteroidaceae; | | | | | | | | | |
| Bacteroides; Bacteroides dorei | | | | | | | | | |
| Bacteria; Proteobacteria; | 21 | 0.006459 | 34.153918 | 0.000020 | 0.000091 | 0.140167 | 0.000693 | 0.003897 | 0.506108 |
| Betaproteobacteria; Other; | | | | | | | | | |
| Other; Other; Other | | | | | | | | | |
| Bacteria; Bacteroidetes; | 22 | 0.006368 | 1.638540 | 0.061864 | 0.066323 | 0.995816 | 0.101366 | 0.098052 | 0.986038 |
| Bacteroidia; Bacteroidales; | | | | | | | | | |
| Bacteroidaceae; Bacteroides; Other | | | | | | | | | |
| Bacteria; Firmicutes; Clostridia; | 23 | 0.006271 | 0.395539 | 0.004287 | 0.008920 | 0.938285 | 0.001696 | 0.003399 | 0.902269 |
| Clostridiales; Lachnospiraceae; | | | | | | | | | |
| Ruminococcus2; Other | | | | | | | | | |
| Bacteria; Firmicutes; Clostridia; | 24 | 0.006199 | 0.496491 | 0.006404 | 0.011919 | 0.855649 | 0.003180 | 0.008982 | 0.766143 |
| Clostridiales; Ruminococcaceae; | | | | | | | | | |
| Ruminococcus; | | | | | | | | | |
| Ruminococcus faecis | | | | | | | | | |
| Bacteria; Actinobacteria; | 25 | 0.006065 | 1.861459 | 0.010770 | 0.034421 | 0.756276 | 0.020048 | 0.036905 | 0.956370 |
| Actinobacteria; Bifidobacteriales; | | | | | | | | | |
| Bifidobacteriaceae; | | | | | | | | | |
| Bifidobacterium; Other | | | | | | | | | |
| Bacteria; Firmicutes; Clostridia; | 26 | 0.005992 | 2.783800 | 0.000009 | 0.000050 | 0.106695 | 0.000024 | 0.000061 | 0.446771 |
| Clostridiales; Ruminococcaceae; | | | | | | | | | |
| Pseudoflavonifractor; Other | | | | | | | | | |
| Bacteria; Firmicutes; Clostridia; | 27 | 0.005931 | 1.308143 | 0.041475 | 0.042224 | 0.967573 | 0.054255 | 0.057663 | 0.979058 |
| Clostridiales; Ruminococcaceae; | | | | | | | | | |
| Faecalibacterium; Other | | | | | | | | | |
| Bacteria; Firmicutes; Clostridia; | 28 | 0.005856 | 2.611655 | 0.002631 | 0.004823 | 0.891213 | 0.006872 | 0.009133 | 0.952880 |
| Clostridiales; Lachnospiraceae; | | | | | | | | | |
| Roseburia; Other | | | | | | | | | |
| Bacteria; Proteobacteria; | 29 | 0.005751 | 15.517704 | 0.000078 | 0.000419 | 0.219665 | 0.001207 | 0.006012 | 0.596859 |
| Betaproteobacteria; Burkholderiales; | | | | | | | | | |
| Other; Other; Other | | | | | | | | | |
| Bacteria; Bacteroidetes; Bacteroidia; | 30 | 0.005694 | 0.440139 | 0.001533 | 0.004041 | 0.702929 | 0.000675 | 0.003398 | 0.359511 |
| Bacteroidales; Bacteroidaceae; | | | | | | | | | |
| Bacteroides; Bacteroides ovatus | | | | | | | | | |
| Bacteria; Firmicutes; Clostridia; | 31 | 0.005620 | 0.639836 | 0.011136 | 0.014246 | 0.933054 | 0.007125 | 0.010254 | 0.888307 |
| Clostridiales; Lachnospiraceae; | | | | | | | | | |
| Fusicatenibacter; | | | | | | | | | |
| Fusicatenibacter saccharivorans | | | | | | | | | |
| Bacteria; Firmicutes; Clostridia; | 32 | 0.005521 | 0.564502 | 0.010075 | 0.018327 | 0.817992 | 0.005688 | 0.011516 | 0.780105 |
| Clostridiales; Ruminococcaceae; | | | | | | | | | |
| Gemmiger; Gemmiger formicilis | | | | | | | | | |
| Bacteria; Firmicutes; Clostridia; | 33 | 0.005497 | 7.294859 | 0.000261 | 0.001295 | 0.320084 | 0.001907 | 0.007485 | 0.673647 |
| Clostridiales; Peptostreptococcaceae; | | | | | | | | | |
| Romboutsia; Other | | | | | | | | | |
| Bacteria; Actinobacteria; | 34 | 0.005403 | 0.619451 | 0.010594 | 0.025323 | 0.555439 | 0.006563 | 0.018823 | 0.834206 |
| Coriobacteriia; Coriobacteriales; | | | | | | | | | |
| Coriobacteriaceae; Collinsella; | | | | | | | | | |
| Collinsella aerofaciens | | | | | | | | | |
| Bacteria; Firmicutes; Clostridia; | 35 | 0.005320 | 2.137281 | 0.000185 | 0.000631 | 0.540795 | 0.000396 | 0.001292 | 0.815009 |
| Clostridiales; Ruminococcaceae; | | | | | | | | | |
| Flavonifractor; Other | | | | | | | | | |
| Bacteria; Proteobacteria; Other; | 36 | 0.005199 | 9.024774 | 0.000039 | 0.000258 | 0.177824 | 0.000349 | 0.001180 | 0.549738 |
| Other; Other; Other; Other | | | | | | | | | |
| Bacteria; Firmicutes; | 37 | 0.005019 | 0.862352 | 0.000031 | 0.000218 | 0.186192 | 0.000027 | 0.000063 | 0.530541 |
| Erysipelotrichia; Erysipelotrichales; | | | | | | | | | |
| Erysipelotrichaceae; Holdemania; Other | | | | | | | | | |
| Bacteria; Firmicutes; Clostridia; | 38 | 0.005008 | 0.748738 | 0.000058 | 0.000380 | 0.228033 | 0.000043 | 0.000082 | 0.589878 |
| Other; Other; Other; Other | | | | | | | | | |
| Bacteria; Actinobacteria; Actinobacteria; | 39 | 0.004908 | 8.238295 | 0.000001 | 0.000010 | 0.032427 | 0.000010 | 0.000026 | 0.312391 |
| Bifidobacteriales; Bifidobacteriaceae; | | | | | | | | | |
| Bifidobacterium; | | | | | | | | | |
| Bifidobacterium boum | | | | | | | | | |
| Bacteria; Firmicutes; Clostridia; | 40 | 0.004854 | 1.549031 | 0.018787 | 0.024996 | 0.982218 | 0.029101 | 0.045538 | 0.984293 |
| Clostridiales; Ruminococcaceae; | | | | | | | | | |
| Other; Other | | | | | | | | | |
| Bacteria; Actinobacteria; | 41 | 0.004831 | 1.322599 | 0.012432 | 0.030734 | 0.647490 | 0.016442 | 0.046861 | 0.406632 |
| Actinobacteria; Bifidobacteriales; | | | | | | | | | |
| Bifidobacteriaceae; Bifidobacterium; | | | | | | | | | |
| Bifidobacterium adolescentis | | | | | | | | | |
| Bacteria; Bacteroidetes; | 42 | 0.004787 | 3.407793 | 0.000153 | 0.001465 | 0.195607 | 0.000522 | 0.001727 | 0.525305 |
| Bacteroidia; Bacteroidales; | | | | | | | | | |
| Rikenellaceae; Other; Other | | | | | | | | | |
| Bacteria; Bacteroidetes; | 43 | 0.004682 | 0.181735 | 0.000507 | 0.001799 | 0.414226 | 0.000092 | 0.000652 | 0.155323 |
| Bacteroidia; Bacteroidales; | | | | | | | | | |
| Bacteroidaceae; Bacteroides; | | | | | | | | | |
| Bacteroides koreensis | | | | | | | | | |
| Bacteria; Firmicutes; Clostridia; | 44 | 0.004511 | 0.768938 | 0.001974 | 0.005469 | 0.876569 | 0.001518 | 0.002170 | 0.855148 |
| Clostridiales; Lachnospiraceae; | | | | | | | | | |
| Fusicatenibacter; Other | | | | | | | | | |
| Bacteria; Actinobacteria; | 45 | 0.004450 | 4.165216 | 0.000034 | 0.000239 | 0.065900 | 0.000142 | 0.000505 | 0.380454 |
| Coriobacteriia; Eggerthellales; | | | | | | | | | |
| Eggerthellaceae; Other; Other | | | | | | | | | |
| Bacteria; Firmicutes; Clostridia; | 46 | 0.004399 | 1.650736 | 0.000018 | 0.000065 | 0.200837 | 0.000030 | 0.000081 | 0.534031 |
| Clostridiales; Lachnospiraceae; | | | | | | | | | |
| Lachnoclostridium; [Clostridium] | | | | | | | | | |
| citroniae | | | | | | | | | |
| Bacteria; Bacteroidetes; Bacteroidia; | 47 | 0.004397 | 0.545097 | 0.002877 | 0.004957 | 0.740586 | 0.001568 | 0.003304 | 0.657941 |
| Bacteroidales; Rikenellaceae; | | | | | | | | | |
| Alistipes; Alistipes shahii | | | | | | | | | |
| Bacteria; Firmicutes; Clostridia; | 48 | 0.004335 | 1.136695 | 0.000680 | 0.002129 | 0.750000 | 0.000773 | 0.002754 | 0.907504 |
| Clostridiales; Lachnospiraceae; | | | | | | | | | |
| Clostridium XIVa; Other | | | | | | | | | |
| Bacteria; Firmicutes; Clostridia; | 49 | 0.004244 | 0.407908 | 0.012988 | 0.031050 | 0.823222 | 0.005298 | 0.012954 | 0.802792 |
| Clostridiales; Ruminococcaceae; | | | | | | | | | |
| Ruminococcus; Other | | | | | | | | | |
| Bacteria; Firmicutes; Clostridia; | 50 | 0.004203 | 1.450417 | 0.013600 | 0.022700 | 0.991632 | 0.019726 | 0.031062 | 0.994764 |
| Clostridiales; Lachnospiraceae; | | | | | | | | | |
| Blautia; Other | | | | | | | | | |
| Bacteria; Bacteroidetes; Bacteroidia; | 51 | 0.004159 | 1.134801 | 0.030320 | 0.042820 | 0.952929 | 0.034407 | 0.044854 | 0.945899 |
| Bacteroidales; Bacteroidaceae; | | | | | | | | | |
| Bacteroides; Bacteroides uniformis | | | | | | | | | |
| Bacteria; Firmicutes; Clostridia; | 52 | 0.004083 | 1.141585 | 0.004115 | 0.005620 | 0.901674 | 0.004698 | 0.008841 | 0.942408 |
| Clostridiales; Lachnospiraceae; | | | | | | | | | |
| Dorea; Other | | | | | | | | | |
| Bacteria; Firmicutes; Bacilli; | 53 | 0.004062 | 1.508857 | 0.007021 | 0.016525 | 0.938285 | 0.010593 | 0.027852 | 0.996510 |
| Lactobacillales; Streptococcaceae; | | | | | | | | | |
| Streptococcus; Other | | | | | | | | | |
| Bacteria; Bacteroidetes; Bacteroidia; | 54 | 0.004054 | 0.173174 | 0.004707 | 0.017805 | 0.510460 | 0.000815 | 0.007838 | 0.212914 |
| Bacteroidales; Bacteroidaceae; | | | | | | | | | |
| Bacteroides; | | | | | | | | | |
| Bacteroides cellulosilyticus | | | | | | | | | |
| Bacteria; Firmicutes; Clostridia; | 55 | 0.004046 | 3.216726 | 0.000004 | 0.000025 | 0.071130 | 0.000014 | 0.000057 | 0.352531 |
| Clostridiales; Eubacteriaceae; | | | | | | | | | |
| Anaerofustis; Other | | | | | | | | | |
| Bacteria; Firmicutes; Clostridia; | 56 | 0.004018 | 0.403832 | 0.004267 | 0.014123 | 0.426778 | 0.001723 | 0.007852 | 0.375218 |
| Clostridiales; Lachnospiraceae; | | | | | | | | | |
| Coprococcus; Coprococcus eutactus | | | | | | | | | |
| Bacteria; Verrucomicrobia; | 57 | 0.003992 | 0.223330 | 0.015218 | 0.049261 | 0.607741 | 0.003399 | 0.014277 | 0.523560 |
| Verrucomicrobiae; Verrucomicrobiales; | | | | | | | | | |
| Akkermansiaceae; Akkermansia; | | | | | | | | | |
| Akkermansia muciniphila | | | | | | | | | |
| Bacteria; Firmicutes; Negativicutes; | 58 | 0.003975 | 0.135223 | 0.007826 | 0.017524 | 0.501046 | 0.001058 | 0.003282 | 0.310646 |
| Acidaminococcales; Acidaminococcaceae; | | | | | | | | | |
| Phascolarctobacterium; | | | | | | | | | |
| Phascolarctobacterium faecium | | | | | | | | | |
| Bacteria; Bacteroidetes; Bacteroidia; | 59 | 0.003945 | 1.635282 | 0.041765 | 0.060175 | 0.972803 | 0.068297 | 0.095683 | 0.961606 |
| Bacteroidales; Bacteroidaceae; | | | | | | | | | |
| Bacteroides; Bacteroides vulgatus | | | | | | | | | |
| Bacteria; Actinobacteria; | 60 | 0.003942 | 1.609195 | 0.000891 | 0.003500 | 0.290795 | 0.001434 | 0.004397 | 0.455497 |
| Actinobacteria; Bifidobacteriales; | | | | | | | | | |
| Bifidobacteriaceae; Bifidobacterium; | | | | | | | | | |
| Bifidobacterium bifidum | | | | | | | | | |
| Bacteria; Firmicutes; Bacilli; Bacillales; | 61 | 0.003933 | 6.963814 | 0.000027 | 0.000075 | 0.261506 | 0.000191 | 0.001477 | 0.525305 |
| Other; Gemella; Other | | | | | | | | | |
| Bacteria; Actinobacteria; | 62 | 0.003871 | 1.566900 | 0.001571 | 0.006672 | 0.605649 | 0.002461 | 0.011990 | 0.390925 |
| Actinobacteria; Bifidobacteriales; | | | | | | | | | |
| Bifidobacteriaceae; Bifidobacterium; | | | | | | | | | |
| Bifidobacterium longum | | | | | | | | | |
| Bacteria; Firmicutes; Clostridia; | 63 | 0.003859 | 1.178341 | 0.000966 | 0.002406 | 0.737448 | 0.001138 | 0.003845 | 0.886562 |
| Clostridiales; Ruminococcaceae; | | | | | | | | | |
| Flavonifractor; | | | | | | | | | |
| Flavonifractor plautii | | | | | | | | | |
| Bacteria; Firmicutes; Clostridia; | 64 | 0.003844 | 58.184490 | 0.000001 | 0.000013 | 0.018828 | 0.000063 | 0.000720 | 0.167539 |
| Clostridiales; Peptostreptococcaceae; | | | | | | | | | |
| Peptostreptococcus; Other | | | | | | | | | |
| Bacteria; Bacteroidetes; Bacteroidia; | 65 | 0.003841 | 0.821235 | 0.036984 | 0.104763 | 0.619247 | 0.030373 | 0.083313 | 0.818499 |
| Bacteroidales; Prevotellaceae; | | | | | | | | | |
| Prevotella; Other | | | | | | | | | |
| Bacteria; Firmicutes; Bacilli; | 66 | 0.003780 | 2.302825 | 0.000013 | 0.000081 | 0.101464 | 0.000029 | 0.000154 | 0.162304 |
| Lactobacillales; Carnobacteriaceae; | | | | | | | | | |
| Granulicatella; | | | | | | | | | |
| Granulicatella adiacens | | | | | | | | | |
| Bacteria; Firmicutes; Clostridia; | 67 | 0.003665 | 1.426542 | 0.000871 | 0.003572 | 0.674686 | 0.001242 | 0.002653 | 0.856894 |
| Clostridiales; Lachnospiraceae; | | | | | | | | | |
| Roseburia; Roseburia hominis | | | | | | | | | |
| Bacteria; Firmicutes; Negativicutes; | 68 | 0.003608 | 1.715495 | 0.000080 | 0.000497 | 0.175732 | 0.000137 | 0.000744 | 0.506108 |
| Veillonellales; Veillonellaceae; | | | | | | | | | |
| Veillonella; Other | | | | | | | | | |
| Bacteria; Firmicutes; Negativicutes; | 69 | 0.003480 | 1.505910 | 0.000733 | 0.004959 | 0.304393 | 0.001104 | 0.004629 | 0.626527 |
| Selenomonadales; Veillonellaceae; | | | | | | | | | |
| Other; Other | | | | | | | | | |
| Bacteria; Firmicutes; Clostridia; | 70 | 0.003380 | 1.209792 | 0.000015 | 0.000126 | 0.134937 | 0.000018 | 0.000055 | 0.406632 |
| Clostridiales; Ruminococcaceae; | | | | | | | | | |
| Anaerotruncus; Other | | | | | | | | | |
| Bacteria; Firmicutes; Tissierellia; | 71 | 0.003344 | 234.211022 | 0.000002 | 0.000013 | 0.034519 | 0.000370 | 0.003594 | 0.211169 |
| Tissierellales; Peptoniphilaceae; | | | | | | | | | |
| Peptoniphilus; Other | | | | | | | | | |
| Bacteria; Actinobacteria; | 72 | 0.003246 | 1.848695 | 0.000223 | 0.000872 | 0.456067 | 0.000412 | 0.002789 | 0.726003 |
| Actinobacteria; Coriobacteriales; | | | | | | | | | |
| Coriobacteriaceae; Other; Other | | | | | | | | | |
| Bacteria; Firmicutes; Clostridia; | 73 | 0.003199 | 0.578007 | 0.016905 | 0.025464 | 0.755230 | 0.009771 | 0.025705 | 0.808028 |
| Clostridiales; Ruminococcaceae; | | | | | | | | | |
| Faecalibacterium; | | | | | | | | | |
| Faecalibacterium prausnitzii | | | | | | | | | |
| Bacteria; Firmicutes; Clostridia; | 74 | 0.003190 | 94.572109 | 0.000009 | 0.000077 | 0.089958 | 0.000893 | 0.012010 | 0.251309 |
| Clostridiales; Clostridiales_Incertae | | | | | | | | | |
| Sedis XI; Parvimonas; Other | | | | | | | | | |
| Bacteria; Firmicutes; Clostridia; | 75 | 0.003177 | 0.488163 | 0.000797 | 0.003787 | 0.583682 | 0.000389 | 0.001687 | 0.364747 |
| Clostridiales; Lachnospiraceae; | | | | | | | | | |
| Lachnoclostridium; | | | | | | | | | |
| [Clostridium] bolteae | | | | | | | | | |
| Bacteria; Firmicutes; Clostridia; | 76 | 0.003157 | 1.081872 | 0.007247 | 0.013333 | 0.910042 | 0.007841 | 0.017319 | 0.958115 |
| Clostridiales; Lachnospiraceae; | | | | | | | | | |
| Blautia; Blautia faecis | | | | | | | | | |
| Bacteria; Firmicutes; Clostridia; | 77 | 0.003114 | 1.622099 | 0.002311 | 0.004024 | 0.911088 | 0.003749 | 0.012395 | 0.973822 |
| Clostridiales; Lachnospiraceae; | | | | | | | | | |
| Anaerostipes; Other | | | | | | | | | |
| Bacteria; Bacteroidetes; Bacteroidia; | 78 | 0.003106 | 0.021896 | 0.003770 | 0.017430 | 0.255230 | 0.000083 | 0.000612 | 0.150087 |
| Bacteroidales; Bacteroidaceae; | | | | | | | | | |
| Bacteroides; | | | | | | | | | |
| Bacteroides thetaiotaomicron | | | | | | | | | |
| Bacteria; Actinobacteria; | 79 | 0.003095 | 0.893259 | 0.000083 | 0.000480 | 0.264644 | 0.000074 | 0.000270 | 0.556719 |
| Actinobacteria; Coriobacteriales; | | | | | | | | | |
| Coriobacteriaceae; Eggerthella; Other | | | | | | | | | |
| Bacteria; Firmicutes; Clostridia; | 80 | 0.003089 | 1.623955 | 0.000189 | 0.001137 | 0.438285 | 0.000307 | 0.001139 | 0.687609 |
| Clostridiales; Peptostreptococcaceae; | | | | | | | | | |
| Intestinibacter; Other | | | | | | | | | |
| Bacteria; Firmicutes; Clostridia; | 81 | 0.003087 | 5.888868 | 0.000085 | 0.000380 | 0.253138 | 0.000502 | 0.002402 | 0.553229 |
| Clostridiales; Peptostreptococcaceae; | | | | | | | | | |
| Terrisporobacter; Other | | | | | | | | | |
| Bacteria; Firmicutes; Erysipelotrichia; | 82 | 0.003087 | 38.278503 | 0.000007 | 0.000074 | 0.027197 | 0.000254 | 0.002137 | 0.228621 |
| Erysipelotrichales; Erysipelotrichaceae; | | | | | | | | | |
| Faecalicoccus; Other | | | | | | | | | |
| Bacteria; Bacteroidetes; Bacteroidia; | 83 | 0.003087 | 1.019142 | 0.004785 | 0.007226 | 0.927824 | 0.004877 | 0.008018 | 0.916230 |
| Bacteroidales; Porphyromonadaceae; | | | | | | | | | |
| Parabacteroides; Other | | | | | | | | | |
| Bacteria; Firmicutes; Clostridia; | 84 | 0.003069 | 1.016844 | 0.016348 | 0.020690 | 0.988494 | 0.016624 | 0.022247 | 0.993019 |
| Clostridiales; Other; Other; Other | | | | | | | | | |
| Bacteria; Bacteroidetes; Bacteroidia; | 85 | 0.003049 | 0.825494 | 0.008663 | 0.012407 | 0.783473 | 0.007151 | 0.011051 | 0.792321 |
| Bacteroidales; Rikenellaceae; | | | | | | | | | |
| Alistipes; Alistipes putredinis | | | | | | | | | |
| Bacteria; Firmicutes; Clostridia; | 86 | 0.003014 | 2.221178 | 0.000238 | 0.001005 | 0.315900 | 0.000528 | 0.002699 | 0.488656 |
| Clostridiales; Lachnospiraceae; | | | | | | | | | |
| Blautia; Blautia hydrogenotrophica | | | | | | | | | |
| Bacteria; Bacteroidetes; Bacteroidia; | 87 | 0.002991 | 0.478481 | 0.000405 | 0.006638 | 0.361925 | 0.000194 | 0.000596 | 0.635253 |
| Bacteroidales; Porphyromonadaceae; | | | | | | | | | |
| Other; Other | | | | | | | | | |
| Bacteria; Firmicutes; Clostridia; | 88 | 0.002965 | 0.767881 | 0.017321 | 0.019449 | 0.981172 | 0.013301 | 0.017704 | 0.989529 |
| Clostridiales; Lachnospiraceae; | | | | | | | | | |
| Lachnospiracea_incertae_sedis; Other | | | | | | | | | |
| Bacteria; Actinobacteria; Coriobacteriia; | 89 | 0.002946 | 0.756428 | 0.000809 | 0.004296 | 0.456067 | 0.000612 | 0.002776 | 0.242583 |
| Eggerthellales; Eggerthellaceae; | | | | | | | | | |
| Adlercreutzia; | | | | | | | | | |
| Adlercreutzia equolifaciens | | | | | | | | | |
| Bacteria; Bacteroidetes; Bacteroidia; | 90 | 0.002940 | 0.456826 | 0.001710 | 0.007218 | 0.498954 | 0.000781 | 0.003104 | 0.246073 |
| Bacteroidales; Bacteroidaceae; | | | | | | | | | |
| Bacteroides; Bacteroides finegoldii | | | | | | | | | |
| Bacteria; Actinobacteria; | 91 | 0.002933 | 0.904545 | 0.000101 | 0.000213 | 0.513598 | 0.000091 | 0.000219 | 0.776614 |
| Actinobacteria; Actinomycetales; | | | | | | | | | |
| Actinomycetaceae; | | | | | | | | | |
| Actinomyces; Other | | | | | | | | | |
| Bacteria; Firmicutes; Clostridia; | 92 | 0.002928 | 0.463041 | 0.001179 | 0.001896 | 0.610879 | 0.000546 | 0.001248 | 0.333333 |
| Clostridiales; Lachnospiraceae; | | | | | | | | | |
| Coprococcus; Coprococcus catus | | | | | | | | | |
| Bacteria; Firmicutes; Clostridia; | 93 | 0.002916 | 1.532655 | 0.000265 | 0.001097 | 0.266736 | 0.000407 | 0.001456 | 0.321117 |
| Clostridiales; Ruminococcaceae; | | | | | | | | | |
| Negativibacillus; | | | | | | | | | |
| Negativibacillus massiliensis | | | | | | | | | |
| Bacteria; Firmicutes; Erysipelotrichia; | 94 | 0.002914 | 6.114725 | 0.000012 | 0.000046 | 0.188285 | 0.000076 | 0.000575 | 0.357766 |
| Erysipelotrichales; Erysipelotrichaceae; | | | | | | | | | |
| Solobacterium; | | | | | | | | | |
| Solobacterium moorei | | | | | | | | | |
| Bacteria; Bacteroidetes; Bacteroidia; | 95 | 0.002893 | 0.751197 | 0.008488 | 0.015847 | 0.756276 | 0.006376 | 0.017283 | 0.757417 |
| Bacteroidales; Tannerellaceae; | | | | | | | | | |
| Parabacteroides; | | | | | | | | | |
| Parabacteroides merdae | | | | | | | | | |
| Bacteria; Actinobacteria; | 96 | 0.002887 | 2.537300 | 0.000008 | 0.000039 | 0.114017 | 0.000020 | 0.000047 | 0.420593 |
| Actinobacteria; Other; Other; | | | | | | | | | |
| Other; Other | | | | | | | | | |
| Bacteria; Bacteroidetes; Bacteroidia; | 97 | 0.002880 | 1.254868 | 0.005870 | 0.016013 | 0.892259 | 0.007366 | 0.024075 | 0.872600 |
| Bacteroidales; Rikenellaceae; | | | | | | | | | |
| Alistipes; Other | | | | | | | | | |
| Bacteria; Firmicutes; Negativicutes; | 98 | 0.002871 | 0.472637 | 0.001273 | 0.004421 | 0.533473 | 0.000602 | 0.002122 | 0.382199 |
| Selenomonadales; Acidaminococcaceae; | | | | | | | | | |
| Phascolarctobacterium; Other | | | | | | | | | |
| Bacteria; Bacteroidetes; Bacteroidia; | 99 | 0.002855 | 0.490327 | 0.002446 | 0.004682 | 0.764644 | 0.001199 | 0.002404 | 0.766143 |
| Bacteroidales; Odoribacteraceae; | | | | | | | | | |
| Odoribacter; | | | | | | | | | |
| Odoribacter splanchnicus | | | | | | | | | |
| Bacteria; Firmicutes; Clostridia; | 100 | 0.002854 | 0.711916 | 0.001015 | 0.002715 | 0.754184 | 0.000723 | 0.003535 | 0.757417 |
| Clostridiales; Ruminococcaceae; | | | | | | | | | |
| Not Available; | | | | | | | | | |
| [Clostridium] leptum | | | | | | | | | |

IBD = Inflammatory Bowel Disease; UC = Ulcerative Colitis; Ctrl = Control; RFFR = Random Forest Feature Rank; # = Rank; MDA = Mean Decrease Accuracy; FC = Fold Change; M = Mean; SD = Standard Deviation; DP = Detection Prevalence;

TABLE 14
IBD (Crohn's Disease) vs Control delineation features - (taxonomy)

| Feature | | | | | | | | | |
|---|---|---|---|---|---|---|---|---|---|
| (Taxonomic order: Kingdom; | | | FC | | | | | | |
| Phylum; Class; Order; | RFFR | | CD vs | Abundance (Ctrl) | | | Abundance (CD) | | |
| Family; Genus; Species) | # | MDA | Ctrl | M | SD | DP | M | SD | DP |
| Bacteria; Firmicutes; Clostridia; | 1 | 0.021267 | 0.353537 | 0.027343 | 0.033723 | 0.892259 | 0.009667 | 0.039898 | 0.366876 |
| Clostridiales; Lachnospiraceae; | | | | | | | | | |
| Not Available; | | | | | | | | | |
| [Eubacterium] rectale | | | | | | | | | |
| Bacteria; Firmicutes; Clostridia; | 2 | 0.019342 | 0.567358 | 0.041475 | 0.042224 | 0.967573 | 0.023531 | 0.045680 | 0.830189 |
| Clostridiales; Ruminococcaceae; | | | | | | | | | |
| Faecalibacterium; Other | | | | | | | | | |
| Bacteria; Firmicutes; Clostridia; | 3 | 0.019271 | 0.919620 | 0.018787 | 0.024996 | 0.982218 | 0.017277 | 0.050587 | 0.882600 |
| Clostridiales; Ruminococcaceae; | | | | | | | | | |
| Other; Other | | | | | | | | | |
| Bacteria; Firmicutes; Clostridia; | 4 | 0.018052 | 0.128232 | 0.007228 | 0.030628 | 0.802301 | 0.000927 | 0.006042 | 0.266247 |
| Clostridiales; Peptostreptococcaceae; | | | | | | | | | |
| Romboutsia; Romboutsia timonensis | | | | | | | | | |
| Bacteria; Bacteroidetes; Bacteroidia; | 5 | 0.016697 | 0.204739 | 0.005759 | 0.017531 | 0.798117 | 0.001179 | 0.004416 | 0.274633 |
| Bacteroidales; Bacteroidaceae; | | | | | | | | | |
| Bacteroides; | | | | | | | | | |
| Bacteroides xylanisolvens | | | | | | | | | |
| Bacteria; Firmicutes; Clostridia; | 6 | 0.015216 | 1.141805 | 0.026222 | 0.040290 | 0.913180 | 0.029940 | 0.073071 | 0.467505 |
| Clostridiales; Lachnospiraceae; | | | | | | | | | |
| Blautia; Blautia wexlerae | | | | | | | | | |
| Bacteria; Firmicutes; Clostridia; | 7 | 0.014032 | 13.373641 | 0.000657 | 0.004356 | 0.218619 | 0.008788 | 0.024397 | 0.662474 |
| Clostridiales; Lachnospiraceae; | | | | | | | | | |
| Faecalimonas; | | | | | | | | | |
| Faecalimonas umbilicata | | | | | | | | | |
| Bacteria; Firmicutes; Clostridia; | 8 | 0.013927 | 0.425272 | 0.003574 | 0.006957 | 0.786611 | 0.001520 | 0.010646 | 0.257862 |
| Clostridiales; Lachnospiraceae; | | | | | | | | | |
| Roseburia; Roseburia faecis | | | | | | | | | |
| Bacteria; Firmicutes; Clostridia; | 9 | 0.013662 | 0.073034 | 0.016905 | 0.025464 | 0.755230 | 0.001235 | 0.005413 | 0.417191 |
| Clostridiales; Ruminococcaceae; | | | | | | | | | |
| Faecalibacterium; | | | | | | | | | |
| Faecalibacterium prausnitzii | | | | | | | | | |
| Bacteria; Firmicutes; Clostridia; | 10 | 0.013457 | 610.962669 | 0.000004 | 0.000027 | 0.059623 | 0.002159 | 0.009194 | 0.446541 |
| Clostridiales; Lachnospiraceae; | | | | | | | | | |
| Lachnoclostridium; Other | | | | | | | | | |
| Bacteria; Firmicutes; Clostridia; | 11 | 0.011943 | 0.401796 | 0.002003 | 0.003530 | 0.778243 | 0.000805 | 0.003840 | 0.264151 |
| Clostridiales; Ruminococcaceae; | | | | | | | | | |
| Agathobaculum; | | | | | | | | | |
| Agathobaculum butyriciproducens | | | | | | | | | |
| Bacteria; Firmicutes; Clostridia; | 12 | 0.011450 | 0.503572 | 0.010075 | 0.018327 | 0.817992 | 0.005074 | 0.020778 | 0.582809 |
| Clostridiales; Ruminococcaceae; | | | | | | | | | |
| Gemmiger; Gemmiger formicilis | | | | | | | | | |
| Bacteria; Firmicutes; Clostridia; | 13 | 0.011427 | 0.470210 | 0.011136 | 0.014246 | 0.933054 | 0.005236 | 0.011289 | 0.763103 |
| Clostridiales; Lachnospiraceae; | | | | | | | | | |
| Fusicatenibacter; | | | | | | | | | |
| Fusicatenibacter saccharivorans | | | | | | | | | |
| Bacteria; Firmicutes; Clostridia; | 14 | 0.010838 | 4.504117 | 0.000680 | 0.002129 | 0.750000 | 0.003062 | 0.006122 | 0.907757 |
| Clostridiales; Lachnospiraceae; | | | | | | | | | |
| Clostridium XIVa; Other | | | | | | | | | |
| Bacteria; Firmicutes; Clostridia; | 15 | 0.009696 | 0.234078 | 0.012988 | 0.031050 | 0.823222 | 0.003040 | 0.012851 | 0.563941 |
| Clostridiales; Ruminococcaceae; | | | | | | | | | |
| Ruminococcus; Other | | | | | | | | | |
| Bacteria; Firmicutes; Clostridia; | 16 | 0.008564 | 0.470453 | 0.006404 | 0.011919 | 0.855649 | 0.003013 | 0.010184 | 0.597484 |
| Clostridiales; Ruminococcaceae; | | | | | | | | | |
| Ruminococcus; | | | | | | | | | |
| Ruminococcus faecis | | | | | | | | | |
| Bacteria; Firmicutes; Clostridia; | 17 | 0.008287 | 0.153682 | 0.001179 | 0.001896 | 0.610879 | 0.000181 | 0.000969 | 0.174004 |
| Clostridiales; Lachnospiraceae; | | | | | | | | | |
| Coprococcus; Coprococcus catus | | | | | | | | | |
| Bacteria; Firmicutes; Clostridia; | 18 | 0.007415 | 7.431555 | 0.000797 | 0.003787 | 0.583682 | 0.005926 | 0.016593 | 0.429769 |
| Clostridiales; Lachnospiraceae; | | | | | | | | | |
| Lachnoclostridium; | | | | | | | | | |
| [Clostridium] bolteae | | | | | | | | | |
| Bacteria; Firmicutes; Clostridia; | 19 | 0.007217 | 0.317562 | 0.002414 | 0.007114 | 0.726987 | 0.000767 | 0.003426 | 0.484277 |
| Clostridiales; Ruminococcaceae; | | | | | | | | | |
| Clostridium IV; Other | | | | | | | | | |
| Bacteria; Firmicutes; Clostridia; | 20 | 0.007174 | 0.345504 | 0.005117 | 0.009258 | 0.783473 | 0.001768 | 0.005336 | 0.517820 |
| Clostridiales; Eubacteriaceae; | | | | | | | | | |
| Eubacterium; | | | | | | | | | |
| [Eubacterium] eligens | | | | | | | | | |
| Bacteria; Firmicutes; Clostridia; | 21 | 0.006999 | 0.375879 | 0.003746 | 0.007142 | 0.809623 | 0.001408 | 0.003615 | 0.568134 |
| Clostridiales; Lachnospiraceae; | | | | | | | | | |
| Roseburia; Roseburia inulinivorans | | | | | | | | | |
| Bacteria; Firmicutes; Clostridia; | 22 | 0.006937 | 0.691676 | 0.001974 | 0.005469 | 0.876569 | 0.001365 | 0.003206 | 0.679245 |
| Clostridiales; Lachnospiraceae; | | | | | | | | | |
| Fusicatenibacter; Other | | | | | | | | | |
| Bacteria; Bacteroidetes; Bacteroidia; | 23 | 0.006887 | 1.113245 | 0.026934 | 0.054373 | 0.777197 | 0.029984 | 0.075431 | 0.373166 |
| Bacteroidales; Bacteroidaceae; | | | | | | | | | |
| Bacteroides; Bacteroides dorei | | | | | | | | | |
| Bacteria; Firmicutes; Clostridia; | 24 | 0.006880 | 0.674096 | 0.004544 | 0.010129 | 0.852510 | 0.003063 | 0.008567 | 0.622642 |
| Clostridiales; Lachnospiraceae; | | | | | | | | | |
| Roseburia; Roseburia intestinalis | | | | | | | | | |
| Bacteria; Proteobacteria; | 25 | 0.006862 | 32.288412 | 0.000105 | 0.000705 | 0.231172 | 0.003383 | 0.009955 | 0.624738 |
| Gammaproteobacteria; Other; Other; | | | | | | | | | |
| Other; Other | | | | | | | | | |
| Bacteria; Firmicutes; Clostridia; | 26 | 0.006708 | 4.893537 | 0.003177 | 0.012917 | 0.672594 | 0.015546 | 0.040155 | 0.903564 |
| Clostridiales; Lachnospiraceae; | | | | | | | | | |
| Blautia; [Ruminococcus] gnavus | | | | | | | | | |
| Bacteria; Bacteroidetes; Bacteroidia; | 27 | 0.006671 | 0.182477 | 0.002877 | 0.004957 | 0.740586 | 0.000525 | 0.001528 | 0.452830 |
| Bacteroidales; Rikenellaceae; | | | | | | | | | |
| Alistipes; Alistipes shahii | | | | | | | | | |
| Bacteria; Firmicutes; Bacilli; | 28 | 0.006558 | 12.509891 | 0.000034 | 0.000231 | 0.221757 | 0.000420 | 0.002511 | 0.628931 |
| Lactobacillales; Other; Other; Other | | | | | | | | | |
| Bacteria; Actinobacteria; | 29 | 0.006544 | 9.310713 | 0.000004 | 0.000028 | 0.060669 | 0.000040 | 0.000108 | 0.392034 |
| Actinobacteria; Actinomycetales; | | | | | | | | | |
| Actinomycetaceae; Other; Other | | | | | | | | | |
| Bacteria; Firmicutes; Clostridia; | 30 | 0.006511 | 0.495293 | 0.007882 | 0.013361 | 0.888075 | 0.003904 | 0.011642 | 0.710692 |
| Clostridiales; Lachnospiraceae; | | | | | | | | | |
| Blautia; Blautia obeum | | | | | | | | | |
| Bacteria; Bacteroidetes; Bacteroidia; | 31 | 0.006485 | 0.950779 | 0.004785 | 0.007226 | 0.927824 | 0.004550 | 0.011480 | 0.763103 |
| Bacteroidales; Porphyromonadaceae; | | | | | | | | | |
| Parabacteroides; Other | | | | | | | | | |
| Bacteria; Proteobacteria; Other; | 32 | 0.006401 | 27.580889 | 0.000039 | 0.000258 | 0.177824 | 0.001067 | 0.002875 | 0.570231 |
| Other; Other; Other; Other | | | | | | | | | |
| Bacteria; Bacteroidetes; Other; | 33 | 0.006058 | 2.821314 | 0.000343 | 0.002384 | 0.446653 | 0.000969 | 0.002709 | 0.651992 |
| Other; Other; Other; Other | | | | | | | | | |
| Bacteria; Proteobacteria; | 34 | 0.006018 | 109.541112 | 0.000321 | 0.002652 | 0.202929 | 0.035184 | 0.110490 | 0.547170 |
| Gammaproteobacteria; Enterobacterales; | | | | | | | | | |
| Other; Other; Other | | | | | | | | | |
| Bacteria; Proteobacteria; | 35 | 0.005918 | 405.991255 | 0.000000 | 0.000002 | 0.013598 | 0.000052 | 0.000161 | 0.266247 |
| Gammaproteobacteria; Enterobacterales; | | | | | | | | | |
| Enterobacteriaceae; Rosenbergiella; | | | | | | | | | |
| Rosenbergiella collisarenosi | | | | | | | | | |
| Bacteria; Bacteroidetes; Bacteroidia; | 36 | 0.005856 | 0.550090 | 0.001533 | 0.004041 | 0.702929 | 0.000843 | 0.005642 | 0.310273 |
| Bacteroidales; Bacteroidaceae; | | | | | | | | | |
| Bacteroides; Bacteroides ovatus | | | | | | | | | |
| Bacteria; Firmicutes; Clostridia; | 37 | 0.005826 | 0.491817 | 0.001354 | 0.004548 | 0.874477 | 0.000666 | 0.001734 | 0.635220 |
| Clostridiales; Ruminococcaceae; | | | | | | | | | |
| Ruthenibacterium; | | | | | | | | | |
| Ruthenibacterium lactatiformans | | | | | | | | | |
| Bacteria; Proteobacteria; | 38 | 0.005781 | 530.985698 | 0.000000 | 0.000001 | 0.007322 | 0.000037 | 0.000115 | 0.247379 |
| Gammaproteobacteria; Enterobacterales; | | | | | | | | | |
| Enterobacteriaceae; Rosenbergiella; | | | | | | | | | |
| Rosenbergiella nectarea | | | | | | | | | |
| Bacteria; Firmicutes; Clostridia; | 39 | 0.005758 | 0.649332 | 0.007247 | 0.013333 | 0.910042 | 0.004706 | 0.011591 | 0.794549 |
| Clostridiales; Lachnospiraceae; | | | | | | | | | |
| Blautia; Blautia faecis | | | | | | | | | |
| Bacteria; Bacteroidetes; Bacteroidia; | 40 | 0.005455 | 1.112833 | 0.041765 | 0.060175 | 0.972803 | 0.046477 | 0.081902 | 0.897275 |
| Bacteroidales; Bacteroidaceae; | | | | | | | | | |
| Bacteroides; Bacteroides vulgatus | | | | | | | | | |
| Bacteria; Firmicutes; Clostridia; | 41 | 0.005404 | 0.527515 | 0.001015 | 0.002715 | 0.754184 | 0.000536 | 0.002380 | 0.469602 |
| Clostridiales; Ruminococcaceae; | | | | | | | | | |
| Not Available; [Clostridium] | | | | | | | | | |
| leptum | | | | | | | | | |
| Bacteria; Other; Other; Other; | 42 | 0.005278 | 2.486706 | 0.005479 | 0.021341 | 0.819038 | 0.013624 | 0.027468 | 0.920335 |
| Other; Other; Other | | | | | | | | | |
| Bacteria; Firmicutes; Negativicutes; | 43 | 0.005228 | 18.334440 | 0.000307 | 0.004018 | 0.348326 | 0.005635 | 0.031520 | 0.643606 |
| Selenomonadales; Veillonellaceae; | | | | | | | | | |
| Veillonella; Other | | | | | | | | | |
| Bacteria; Bacteroidetes; Bacteroidia; | 44 | 0.005204 | 0.134867 | 0.004707 | 0.017805 | 0.510460 | 0.000635 | 0.004389 | 0.138365 |
| Bacteroidales; Bacteroidaceae; | | | | | | | | | |
| Bacteroides; | | | | | | | | | |
| Bacteroides cellulosilyticus | | | | | | | | | |
| Bacteria; Firmicutes; | 45 | 0.005122 | 2.128493 | 0.000772 | 0.001531 | 0.731172 | 0.001644 | 0.003814 | 0.863732 |
| Other; Other; Other; Other; Other | | | | | | | | | |
| Bacteria; Bacteroidetes; Bacteroidia; | 46 | 0.005072 | 1.746902 | 0.001006 | 0.004950 | 0.701883 | 0.001758 | 0.002928 | 0.761006 |
| Bacteroidales; Other; Other; Other | | | | | | | | | |
| Bacteria; Firmicutes; Clostridia; | 47 | 0.005022 | 2.293744 | 0.013600 | 0.022700 | 0.991632 | 0.031195 | 0.061840 | 0.972746 |
| Clostridiales; Lachnospiraceae; | | | | | | | | | |
| Blautia; Other | | | | | | | | | |
| Bacteria; Firmicutes; Negativicutes; | 48 | 0.005021 | 9.433882 | 0.000201 | 0.002675 | 0.170502 | 0.001899 | 0.008474 | 0.486373 |
| Selenomonadales; Acidaminococcaceae; | | | | | | | | | |
| Acidaminococcus; Other | | | | | | | | | |
| Bacteria; Bacteroidetes; Bacteroidia; | 49 | 0.004900 | 1.590217 | 0.061864 | 0.066323 | 0.995816 | 0.098377 | 0.108029 | 0.958071 |
| Bacteroidales; Bacteroidaceae; | | | | | | | | | |
| Bacteroides; Other | | | | | | | | | |
| Bacteria; Firmicutes; Clostridia; | 50 | 0.004828 | 1.277811 | 0.004115 | 0.005620 | 0.901674 | 0.005258 | 0.012151 | 0.813417 |
| Clostridiales; Lachnospiraceae; | | | | | | | | | |
| Dorea; Other | | | | | | | | | |
| Bacteria; Bacteroidetes; Bacteroidia; | 51 | 0.004786 | 0.750583 | 0.030320 | 0.042820 | 0.952929 | 0.022757 | 0.044217 | 0.853249 |
| Bacteroidales; Bacteroidaceae; | | | | | | | | | |
| Bacteroides; Bacteroides uniformis | | | | | | | | | |
| Bacteria; Firmicutes; Clostridia; | 52 | 0.004728 | 3.159827 | 0.000114 | 0.000526 | 0.312762 | 0.000359 | 0.001053 | 0.534591 |
| Clostridiales; Lachnospiraceae; | | | | | | | | | |
| Lachnoclostridium; | | | | | | | | | |
| [Clostridium] aldenense | | | | | | | | | |
| Bacteria; Firmicutes; Negativicutes; | 53 | 0.004647 | 21.095184 | 0.000080 | 0.000497 | 0.175732 | 0.001681 | 0.012096 | 0.526205 |
| Veillonellales; Veillonellaceae; | | | | | | | | | |
| Veillonella; Other | | | | | | | | | |
| Bacteria; Firmicutes; Clostridia; | 54 | 0.004562 | 0.787579 | 0.015276 | 0.024437 | 0.915272 | 0.012031 | 0.022706 | 0.796646 |
| Clostridiales; Lachnospiraceae; | | | | | | | | | |
| Blautia; Blautia luti | | | | | | | | | |
| Bacteria; Firmicutes; Clostridia; | 55 | 0.004460 | 8.703254 | 0.001034 | 0.005783 | 0.419456 | 0.008997 | 0.037618 | 0.356394 |
| Clostridiales; Lachnospiraceae; | | | | | | | | | |
| Blautia; Blautia hominis | | | | | | | | | |
| Bacteria; Verrucomicrobia; | 56 | 0.004385 | 0.450202 | 0.015218 | 0.049261 | 0.607741 | 0.006851 | 0.034258 | 0.419287 |
| Verrucomicrobiae; Verrucomicrobiales; | | | | | | | | | |
| Akkermansiaceae; Akkermansia; | | | | | | | | | |
| Akkermansia muciniphila | | | | | | | | | |
| Bacteria; Firmicutes; Clostridia; | 57 | 0.004289 | 0.861990 | 0.017321 | 0.019449 | 0.981172 | 0.014931 | 0.026591 | 0.964361 |
| Clostridiales; Lachnospiraceae; | | | | | | | | | |
| Lachnospiracea_incertae_sedis; Other | | | | | | | | | |
| Bacteria; Firmicutes; | 58 | 0.004233 | 31.793703 | 0.000010 | 0.000082 | 0.121339 | 0.000303 | 0.002042 | 0.450734 |
| Bacilli; Other; Other; Other; Other | | | | | | | | | |
| Bacteria; Bacteroidetes; Bacteroidia; | 59 | 0.004200 | 0.372854 | 0.008663 | 0.012407 | 0.783473 | 0.003230 | 0.007353 | 0.635220 |
| Bacteroidales; Rikenellaceae; | | | | | | | | | |
| Alistipes; Alistipes putredinis | | | | | | | | | |
| Bacteria; Actinobacteria; | 60 | 0.004159 | 17.534762 | 0.000001 | 0.000010 | 0.032427 | 0.000021 | 0.000081 | 0.264151 |
| Actinobacteria; Bifidobacteriales; | | | | | | | | | |
| Bifidobacteriaceae; Bifidobacterium; | | | | | | | | | |
| Bifidobacterium boum | | | | | | | | | |
| Bacteria; Firmicutes; Clostridia; | 61 | 0.004122 | 0.872797 | 0.004287 | 0.008920 | 0.938285 | 0.003742 | 0.012570 | 0.807218 |
| Clostridiales; Lachnospiraceae; | | | | | | | | | |
| Ruminococcus2; Other | | | | | | | | | |
| Bacteria; Firmicutes; Clostridia; | 62 | 0.004093 | 1.235607 | 0.002631 | 0.004823 | 0.891213 | 0.003251 | 0.006831 | 0.809224 |
| Clostridiales; Lachnospiraceae; | | | | | | | | | |
| Roseburia; Other | | | | | | | | | |
| Bacteria; Bacteroidetes; Bacteroidia; | 63 | 0.003996 | 0.012358 | 0.003770 | 0.017430 | 0.255230 | 0.000047 | 0.000316 | 0.058700 |
| Bacteroidales; Bacteroidaceae; | | | | | | | | | |
| Bacteroides; | | | | | | | | | |
| Bacteroides thetaiotaomicron | | | | | | | | | |
| Bacteria; Bacteroidetes; Bacteroidia; | 64 | 0.003890 | 0.216079 | 0.002446 | 0.004682 | 0.764644 | 0.000529 | 0.001052 | 0.517820 |
| Bacteroidales; Odoribacteraceae; | | | | | | | | | |
| Odoribacter; | | | | | | | | | |
| Odoribacter splanchnicus | | | | | | | | | |
| Bacteria; Firmicutes; Clostridia; | 65 | 0.003819 | 0.257589 | 0.000465 | 0.001909 | 0.313808 | 0.000120 | 0.001787 | 0.077568 |
| Clostridiales; Ruminococcaceae; | | | | | | | | | |
| Neglecta; Neglecta timonensis | | | | | | | | | |
| Bacteria; Bacteroidetes; Bacteroidia; | 66 | 0.003787 | 0.534574 | 0.005870 | 0.016013 | 0.892259 | 0.003138 | 0.008754 | 0.733753 |
| Bacteroidales; Rikenellaceae; | | | | | | | | | |
| Alistipes; Other | | | | | | | | | |
| Bacteria; Verrucomicrobia; | 67 | 0.003785 | 0.842219 | 0.008451 | 0.033406 | 0.643305 | 0.007117 | 0.045593 | 0.549266 |
| Verrucomicrobiae; Verrucomicrobiales; | | | | | | | | | |
| Verrucomicrobiaceae; | | | | | | | | | |
| Akkermansia; Other | | | | | | | | | |
| Bacteria; Actinobacteria; | 68 | 0.003754 | 2.440230 | 0.010770 | 0.034421 | 0.756276 | 0.026282 | 0.066768 | 0.867925 |
| Actinobacteria; Bifidobacteriales; | | | | | | | | | |
| Bifidobacteriaceae; | | | | | | | | | |
| Bifidobacterium; Other | | | | | | | | | |
| Bacteria; Proteobacteria; | 69 | 0.003701 | 160.305085 | 0.000000 | 0.000004 | 0.014644 | 0.000049 | 0.000166 | 0.257862 |
| Gammaproteobacteria; Enterobacterales; | | | | | | | | | |
| Enterobacteriaceae; Cedecea; Other | | | | | | | | | |
| Bacteria; Firmicutes; Clostridia; | 70 | 0.003675 | 0.193382 | 0.004267 | 0.014123 | 0.426778 | 0.000825 | 0.004840 | 0.259958 |
| Clostridiales; Lachnospiraceae; | | | | | | | | | |
| Coprococcus; | | | | | | | | | |
| Coprococcus eutactus | | | | | | | | | |
| Bacteria; Firmicutes; Clostridia; | 71 | 0.003648 | 0.857013 | 0.016148 | 0.023196 | 0.965481 | 0.013839 | 0.023652 | 0.844864 |
| Clostridiales; Lachnospiraceae; | | | | | | | | | |
| Anaerostipes; | | | | | | | | | |
| Anaerostipes hadrus | | | | | | | | | |
| Bacteria; Firmicutes; Clostridia; | 72 | 0.003636 | 7.392951 | 0.000028 | 0.000117 | 0.142259 | 0.000209 | 0.000857 | 0.341719 |
| Clostridiales; Ruminococcaceae; | | | | | | | | | |
| Intestinimonas; Other | | | | | | | | | |
| Bacteria; Bacteroidetes; Bacteroidia; | 73 | 0.003635 | 0.403537 | 0.008488 | 0.015847 | 0.756276 | 0.003425 | 0.009210 | 0.645702 |
| Bacteroidales; Tannerellaceae; | | | | | | | | | |
| Parabacteroides; | | | | | | | | | |
| Parabacteroides merdae | | | | | | | | | |
| Bacteria; Firmicutes; | 74 | 0.003602 | 0.190737 | 0.001388 | 0.005857 | 0.634937 | 0.000265 | 0.000947 | 0.387841 |
| Erysipelotrichia; Erysipelotrichales; | | | | | | | | | |
| Erysipelotrichaceae; Turicibacter; | | | | | | | | | |
| Turicibacter sanguinis | | | | | | | | | |
| Bacteria; Firmicutes; Clostridia; | 75 | 0.003402 | 6.892533 | 0.000261 | 0.001295 | 0.320084 | 0.001801 | 0.007718 | 0.461216 |
| Clostridiales; Peptostreptococcaceae; | | | | | | | | | |
| Romboutsia; Other | | | | | | | | | |
| Bacteria; Fusobacteria; | 76 | 0.003364 | 29.489644 | 0.000183 | 0.002664 | 0.125523 | 0.005410 | 0.027960 | 0.385744 |
| Fusobacteriia; Fusobacteriales; | | | | | | | | | |
| Fusobacteriaceae; Fusobacterium; | | | | | | | | | |
| Other | | | | | | | | | |
| Bacteria; Firmicutes; Clostridia; | 77 | 0.003349 | 1.898595 | 0.002311 | 0.004024 | 0.911088 | 0.004388 | 0.009337 | 0.882600 |
| Clostridiales; Lachnospiraceae; | | | | | | | | | |
| Anaerostipes; Other | | | | | | | | | |
| Bacteria; Firmicutes; Bacilli; | 78 | 0.003317 | 0.342737 | 0.003012 | 0.006257 | 0.688285 | 0.001032 | 0.003323 | 0.475891 |
| Lactobacillales; Lactobacillus; | | | | | | | | | |
| Lactobacillus; | | | | | | | | | |
| Lactobacillus rogosae | | | | | | | | | |
| Bacteria; Firmicutes; Negativicutes; | 79 | 0.003164 | 2.557738 | 0.000733 | 0.004959 | 0.304393 | 0.001876 | 0.013139 | 0.626834 |
| Selenomonadales; Veillonellaceae; | | | | | | | | | |
| Other; Other | | | | | | | | | |
| Bacteria; Bacteroidetes; Bacteroidia; | 80 | 0.003149 | 0.386643 | 0.000507 | 0.001799 | 0.414226 | 0.000196 | 0.001164 | 0.180294 |
| Bacteroidales; Bacteroidaceae; | | | | | | | | | |
| Bacteroides; | | | | | | | | | |
| Bacteroides koreensis | | | | | | | | | |
| Bacteria; Firmicutes; Clostridia; | 81 | 0.003135 | 0.622601 | 0.016348 | 0.020690 | 0.988494 | 0.010178 | 0.013332 | 0.981132 |
| Clostridiales; Other; Other; Other | | | | | | | | | |
| Bacteria; Firmicutes; Bacilli; | 82 | 0.003133 | 2.358423 | 0.007021 | 0.016525 | 0.938285 | 0.016557 | 0.041320 | 0.966457 |
| Lactobacillales; Streptococcaceae; | | | | | | | | | |
| Streptococcus; Other | | | | | | | | | |
| Bacteria; Firmicutes; Clostridia; | 83 | 0.003126 | 1.147884 | 0.047176 | 0.039074 | 0.991632 | 0.054152 | 0.054363 | 0.985325 |
| Clostridiales; Lachnospiraceae; Other; Other | | | | | | | | | |
| Bacteria; Firmicutes; Clostridia; | 84 | 0.003116 | 0.492971 | 0.000628 | 0.002760 | 0.642259 | 0.000309 | 0.001143 | 0.339623 |
| Clostridiales; Eubacteriaceae; | | | | | | | | | |
| Eubacterium; Other | | | | | | | | | |
| Bacteria; Firmicutes; Clostridia; | 85 | 0.003108 | 2.604901 | 0.000185 | 0.000631 | 0.540795 | 0.000482 | 0.002604 | 0.670860 |
| Clostridiales; Ruminococcaceae; | | | | | | | | | |
| Flavonifractor; Other | | | | | | | | | |
| Bacteria; Firmicutes; Clostridia; | 86 | 0.003081 | 0.129616 | 0.000107 | 0.000340 | 0.281381 | 0.000014 | 0.000074 | 0.085954 |
| Clostridiales; Not Available; | | | | | | | | | |
| Colidextribacter; | | | | | | | | | |
| Colidextribacter massiliensis | | | | | | | | | |
| Bacteria; Fusobacteria; Fusobacteriia; | 87 | 0.003058 | 44.941541 | 0.000074 | 0.001650 | 0.123431 | 0.003326 | 0.018044 | 0.371069 |
| Fusobacteriales; Fusobacteriaceae; | | | | | | | | | |
| Fusobacterium; | | | | | | | | | |
| Fusobacterium nucleatum | | | | | | | | | |
| Archaea; Euryarchaeota; Methanobacteria; | 88 | 0.003030 | 0.091235 | 0.005034 | 0.015858 | 0.429916 | 0.000459 | 0.004455 | 0.19961 |
| Methanobacteriales; Methanobacteriaceae; | | | | | | | | | |
| Methanobrevibacter; | | | | | | | | | |
| Methanobrevibacter smithii | | | | | | | | | |
| Bacteria; Firmicutes; Negativicutes; | 89 | 0.003013 | 69.630050 | 0.000016 | 0.000306 | 0.055439 | 0.001107 | 0.006836 | 0.295597 |
| Veillonellales; Veillonellaceae; | | | | | | | | | |
| Megasphaera; | | | | | | | | | |
| Megasphaera micronuciformis | | | | | | | | | |
| Bacteria; Actinobacteria; Coriobacteriia; | 90 | 0.003011 | 0.695938 | 0.000809 | 0.004296 | 0.456067 | 0.000563 | 0.003588 | 0.165618 |
| Eggerthellales; Eggerthellaceae; | | | | | | | | | |
| Adlercreutzia; | | | | | | | | | |
| Adlercreutzia equolifaciens | | | | | | | | | |
| Bacteria; Proteobacteria; | 91 | 0.002951 | 3.229609 | 0.002611 | 0.011365 | 0.652720 | 0.008432 | 0.025487 | 0.842767 |
| Gammaproteobacteria; Enterobacteriales; | | | | | | | | | |
| Enterobacteriaceae; Other; Other | | | | | | | | | |
| Bacteria; Proteobacteria; | 92 | 0.002951 | 0.484266 | 0.004015 | 0.012731 | 0.673640 | 0.001944 | 0.007159 | 0.461216 |
| Betaproteobacteria; Burkholderiales; | | | | | | | | | |
| Sutterellaceae; Parasutterella; | | | | | | | | | |
| Parasutterella excrementihominis | | | | | | | | | |
| Bacteria; Firmicutes; Clostridia; | 93 | 0.002938 | 0.356917 | 0.000292 | 0.040 | 0.543933 | 0.000104 | 0.000540 | 0.310273 |
| Clostridiales; Ruminococcaceae; | | | | | | | | | |
| Oscillibacter; Other | | | | | | | | | |
| Bacteria; Firmicutes; Clostridia; | 94 | 0.002903 | 2.271406 | 0.000189 | 0.001137 | 0.438285 | 0.000429 | 0.001476 | 0.595388 |
| Clostridiales; Peptostreptococcaceae; | | | | | | | | | |
| Intestinibacter; Other | | | | | | | | | |
| Bacteria; Firmicutes; Clostridia; | 95 | 0.002872 | 1.299070 | 0.000891 | 0.003813 | 0.756276 | 0.001158 | 0.003329 | 0.706499 |
| Clostridiales; Peptostreptococcaceae; | | | | | | | | | |
| Other; Other | | | | | | | | | |
| Bacteria; Firmicutes; Negativicutes; | 96 | 0.002866 | 0.399205 | 0.007826 | 0.017524 | 0.501046 | 0.003124 | 0.014551 | 0.272537 |
| Acidaminococcales; Acidaminococcaceae; | | | | | | | | | |
| Phascolarctobacterium; | | | | | | | | | |
| Phascolarctobacterium faecium | | | | | | | | | |
| Bacteria; Bacteroidetes; Bacteroidia; | 97 | 0.002837 | 0.609067 | 0.002972 | 0.006360 | 0.816946 | 0.001810 | 0.003865 | 0.706499 |
| Bacteroidales; Rikenellaceae; | | | | | | | | | |
| Alistipes; Other | | | | | | | | | |
| Bacteria; Actinobacteria; Coriobacteriia; | 98 | 0.002833 | 1.195718 | 0.000541 | 0.001754 | 0.474895 | 0.000647 | 0.001878 | 0.568134 |
| Eggerthellales; Eggerthellaceae; | | | | | | | | | |
| Eggerthella; Eggerthella lenta | | | | | | | | | |
| Bacteria; Firmicutes; Erysipelotrichia; | 99 | 0.002761 | 0.505132 | 0.000128 | 0.000258 | 0.525105 | 0.000065 | 0.000259 | 0.283019 |
| Erysipelotrichales; Erysipelotrichaceae; | | | | | | | | | |
| Holdemania; | | | | | | | | | |
| Holdemania filiformis | | | | | | | | | |
| Bacteria; Actinobacteria; Actinobacteria; | 100 | 0.002732 | 0.428909 | 0.012432 | 0.030734 | 0.647490 | 0.005332 | 0.023558 | 0.366876 |
| Bifidobacteriales; Bifidobacteriaceae; | | | | | | | | | |
| Bifidobacterium; | | | | | | | | | |
| Bifidobacterium adolescentis | | | | | | | | | |

IBD = Inflammatory Bowel Disease; CD = Crohn's Disease; Ctrl = Control; RFFR = Random Forest Feature Rank; # = Rank; MDA = Mean Decrease Accuracy; FC = Fold Change; M = Mean; SD = Standard Deviation; DP = Detection Prevalence;

TABLE 15
IBD (Crohn's Disease) vs IBD (Ulcerative Colitis) delineation features - (taxonomy)

| Feature | | | | | | | | | |
|---|---|---|---|---|---|---|---|---|---|
| (Taxonomic order: Kingdom; | | | FC | | | | | | |
| Phylum; Class; Order; | RFFR | | CD vs | Abundance (UC) | | | Abundance (CD) | | |
| Family; Genus; Species) | # | MDA | UC | M | SD | DP | M | SD | DP |
| Bacteria; Firmicutes; | 1 | 0.018811 | 0.593675 | 0.029101 | 0.045538 | 0.984293 | 0.017277 | 0.050587 | 0.882600 |
| Clostridia; Clostridiales; | | | | | | | | | |
| Ruminococcaceae; Other; Other | | | | | | | | | |
| Bacteria; Firmicutes; | 2 | 0.017743 | 21.742695 | 0.000404 | 0.002654 | 0.254799 | 0.008788 | 0.024397 | 0.662474 |
| Clostridia; Clostridiales; Lachnospiraceae; | | | | | | | | | |
| Faecalimonas; | | | | | | | | | |
| Faecalimonas umbilicata | | | | | | | | | |
| Bacteria; Firmicutes; Clostridia; | 3 | 0.015550 | 0.288627 | 0.006126 | 0.010149 | 0.858639 | 0.001768 | 0.005336 | 0.517820 |
| Clostridiales; Eubacteriaceae; | | | | | | | | | |
| Eubacterium; | | | | | | | | | |
| [Eubacterium] eligens | | | | | | | | | |
| Bacteria; Firmicutes; Clostridia; | 4 | 0.014209 | 0.433713 | 0.054255 | 0.057663 | 0.979058 | 0.023531 | 0.045680 | 0.830189 |
| Clostridiales; Ruminococcaceae; | | | | | | | | | |
| Faecalibacterium; Other | | | | | | | | | |
| Bacteria; Firmicutes; Clostridia; | 5 | 0.013744 | 0.126355 | 0.009771 | 0.025705 | 0.808028 | 0.001235 | 0.005413 | 0.417191 |
| Clostridiales; Ruminococcaceae; | | | | | | | | | |
| Faecalibacterium; | | | | | | | | | |
| Faecalibacterium prausnitzii | | | | | | | | | |
| Bacteria; Firmicutes; Clostridia; | 6 | 0.011945 | 0.187625 | 0.000555 | 0.002178 | 0.738220 | 0.000104 | 0.000540 | 0.310273 |
| Clostridiales; Ruminococcaceae; | | | | | | | | | |
| Oscillibacter; Other | | | | | | | | | |
| Bacteria; Firmicutes; Clostridia; | 7 | 0.011905 | 0.384532 | 0.003662 | 0.006283 | 0.898778 | 0.001408 | 0.003615 | 0.568134 |
| Clostridiales; Lachnospiraceae; | | | | | | | | | |
| Roseburia; | | | | | | | | | |
| Roseburia inulinivorans | | | | | | | | | |
| Bacteria; Firmicutes; Clostridia; | 8 | 0.010747 | 0.163329 | 0.004694 | 0.018765 | 0.832461 | 0.000767 | 0.003426 | 0.484277 |
| Clostridiales; Ruminococcaceae; | | | | | | | | | |
| Clostridium IV; Other | | | | | | | | | |
| Bacteria; Firmicutes; Clostridia; | 9 | 0.010424 | 7.269318 | 0.002139 | 0.010083 | 0.790576 | 0.015546 | 0.040155 | 0.903564 |
| Clostridiales; Lachnospiraceae; | | | | | | | | | |
| Blautia; | | | | | | | | | |
| [Ruminococcus] gnavus | | | | | | | | | |
| Bacteria; Firmicutes; Clostridia; | 10 | 0.010408 | 0.496429 | 0.007864 | 0.016597 | 0.928447 | 0.003904 | 0.011642 | 0.710692 |
| Clostridiales; Lachnospiraceae; | | | | | | | | | |
| Blautia; Blautia obeum | | | | | | | | | |
| Bacteria; Firmicutes; Bacilli; | 11 | 0.010367 | 0.321841 | 0.003208 | 0.005786 | 0.781850 | 0.001032 | 0.003323 | 0.475891 |
| Lactobacillales; Lactobacillus; | | | | | | | | | |
| Lactobacillus; | | | | | | | | | |
| Lactobacillus rogosae | | | | | | | | | |
| Bacteria; Firmicutes; | 12 | 0.009628 | 0.257645 | 0.001027 | 0.003252 | 0.741710 | 0.000265 | 0.000947 | 0.387841 |
| Erysipelotrichia; Erysipelotrichales; | | | | | | | | | |
| Erysipelotrichaceae; Turicibacter; | | | | | | | | | |
| Turicibacter sanguinis | | | | | | | | | |
| Bacteria; Firmicutes; Clostridia; | 13 | 0.008948 | 0.600193 | 0.007841 | 0.017319 | 0.958115 | 0.004706 | 0.011591 | 0.794549 |
| Clostridiales; Lachnospiraceae; | | | | | | | | | |
| Blautia; Blautia faecis | | | | | | | | | |
| Bacteria; Firmicutes; Clostridia; | 14 | 0.008864 | 0.777271 | 0.003941 | 0.009222 | 0.895288 | 0.003063 | 0.008567 | 0.622642 |
| Clostridiales; Lachnospiraceae; | | | | | | | | | |
| Roseburia; | | | | | | | | | |
| Roseburia intestinalis | | | | | | | | | |
| Bacteria; Proteobacteria; | 15 | 0.008780 | 4.981155 | 0.001693 | 0.007303 | 0.755672 | 0.008432 | 0.025487 | 0.842767 |
| Gammaproteobacteria; Enterobacteriales; | | | | | | | | | |
| Enterobacteriaceae; Other; Other | | | | | | | | | |
| Bacteria; Firmicutes; Clostridia; | 16 | 0.007946 | 0.991634 | 0.012133 | 0.018460 | 0.951134 | 0.012031 | 0.022706 | 0.796646 |
| Clostridiales; Lachnospiraceae; | | | | | | | | | |
| Blautia; Blautia luti | | | | | | | | | |
| Bacteria; Firmicutes; Clostridia; | 17 | 0.007794 | 3.962467 | 0.000773 | 0.002754 | 0.907504 | 0.003062 | 0.006122 | 0.907757 |
| Clostridiales; Lachnospiraceae; | | | | | | | | | |
| Clostridium XIVa; Other | | | | | | | | | |
| Bacteria; Firmicutes; Clostridia; | 18 | 0.007637 | 0.612288 | 0.016624 | 0.022247 | 0.993019 | 0.010178 | 0.013332 | 0.981132 |
| Clostridiales; Other; Other; Other | | | | | | | | | |
| Bacteria; Firmicutes; Clostridia; | 19 | 0.007377 | 0.473113 | 0.006872 | 0.009133 | 0.952880 | 0.003251 | 0.006831 | 0.809224 |
| Clostridiales; Lachnospiraceae; | | | | | | | | | |
| Roseburia; Other | | | | | | | | | |
| Bacteria; Firmicutes; Clostridia; | 20 | 0.007300 | 0.187357 | 0.004947 | 0.024219 | 0.439791 | 0.000927 | 0.006042 | 0.266247 |
| Clostridiales; Peptostreptococcaceae; | | | | | | | | | |
| Romboutsia; | | | | | | | | | |
| Romboutsia timonensis | | | | | | | | | |
| Bacteria; Bacteroidetes; Bacteroidia; | 21 | 0.006420 | 0.250302 | 0.001805 | 0.004505 | 0.685864 | 0.000452 | 0.001638 | 0.350105 |
| Bacteroidales; Rikenellaceae; | | | | | | | | | |
| Alistipes; Alistipes obesi | | | | | | | | | |
| Bacteria; Firmicutes; Clostridia; | 22 | 0.006218 | 0.727120 | 0.001242 | 0.002653 | 0.856894 | 0.000903 | 0.003434 | 0.576520 |
| Clostridiales; Lachnospiraceae; | | | | | | | | | |
| Roseburia; Roseburia hominis | | | | | | | | | |
| Bacteria; Firmicutes; Clostridia; | 23 | 0.006212 | 0.762807 | 0.004781 | 0.007170 | 0.876091 | 0.003647 | 0.008038 | 0.668'63 |
| Clostridiales; Lachnospiraceae; | | | | | | | | | |
| Coprococcus; Coprococcus comes | | | | | | | | | |
| Bacteria; Firmicutes; Clostridia; | 24 | 0.005971 | 15.223514 | 0.000389 | 0.001687 | 0.364747 | 0.005926 | 0.016593 | 0.429769 |
| Clostridiales; Lachnospiraceae; | | | | | | | | | |
| Lachnoclostridium; | | | | | | | | | |
| [Clostridium] bolteae | | | | | | | | | |
| Bacteria; Firmicutes; Clostridia; | 25 | 0.005950 | 0.734461 | 0.000906 | 0.003383 | 0.895288 | 0.000666 | 0.001734 | 0.635220 |
| Clostridiales; Ruminococcaceae; | | | | | | | | | |
| Ruthenibacterium; | | | | | | | | | |
| Ruthenibacterium lactatiformans | | | | | | | | | |
| Bacteria; Firmicutes; | 26 | 0.005897 | 5.467243 | 0.000463 | 0.002414 | 0.436300 | 0.002529 | 0.009513 | 0.666667 |
| Erysipelotrichia; Erysipelotrichales; | | | | | | | | | |
| Erysipelotrichaceae; Erysipelatoclostridium; | | | | | | | | | |
| Erysipelatoclostridium ramosum | | | | | | | | | |
| Bacteria; Firmicutes; Clostridia; | 27 | 0.005858 | 0.130866 | 0.000024 | 0.000061 | 0.446771 | 0.000003 | 0.000012 | 0.132075 |
| Clostridiales; Ruminococcaceae; | | | | | | | | | |
| Pseudoflavonifractor; Other | | | | | | | | | |
| Bacteria; Firmicutes; Clostridia; | 28 | 0.005730 | 1.119331 | 0.004698 | 0.008841 | 0.942408 | 0.005258 | 0.012151 | 0.813417 |
| Clostridiales; Lachnospiraceae; | | | | | | | | | |
| Dorea; Other | | | | | | | | | |
| Bacteria; Firmicutes; | 29 | 0.005688 | 0.647888 | 0.000100 | 0.000233 | 0.568935 | 0.000065 | 0.000259 | 0.283019 |
| Erysipelotrichia; Erysipelotrichales; | | | | | | | | | |
| Erysipelotrichaceae; Holdemania; | | | | | | | | | |
| Holdemania filiformis | | | | | | | | | |
| Bacteria; Firmicutes; Clostridia; | 30 | 0.005430 | 0.573849 | 0.005298 | 0.012954 | 0.802792 | 0.003040 | 0.012851 | 0.563941 |
| Clostridiales; Ruminococcaceae; | | | | | | | | | |
| Ruminococcus; Other | | | | | | | | | |
| Bacteria; Firmicutes; | 31 | 0.005382 | 4.109247 | 0.000251 | 0.001174 | 0.591623 | 0.001031 | 0.006328 | 0.689727 |
| Erysipelotrichia; Erysipelotrichales; | | | | | | | | | |
| Erysipelotrichaceae; Clostridium XVIII; | | | | | | | | | |
| Other | | | | | | | | | |
| Bacteria; Firmicutes; Clostridia; | 32 | 0.005097 | 0.575653 | 0.003144 | 0.006592 | 0.909250 | 0.001810 | 0.003865 | 0.706499 |
| Clostridiales; Lachnospiraceae; | | | | | | | | | |
| Coprococcus; Other | | | | | | | | | |
| Bacteria; Firmicutes; | 33 | 0.005057 | 15.261253 | 0.000369 | 0.002533 | 0.598604 | 0.005635 | 0.031520 | 0.643606 |
| Negativicutes; Selenomonadales; | | | | | | | | | |
| Veillonellaceae; Veillonella; Other | | | | | | | | | |
| Bacteria; Firmicutes; Clostridia; | 34 | 0.004997 | 0.740979 | 0.000723 | 0.003535 | 0.757417 | 0.000536 | 0.002380 | 0.469602 |
| Clostridiales; Ruminococcaceae; Not | | | | | | | | | |
| Available; [Clostridium] | | | | | | | | | |
| leptum | | | | | | | | | |
| Bacteria; Firmicutes; Clostridia; | 35 | 0.004962 | 0.734891 | 0.007125 | 0.010254 | 0.888307 | 0.005236 | 0.011289 | 0.763103 |
| Clostridiales; Lachnospiraceae; | | | | | | | | | |
| Fusicatenibacter; | | | | | | | | | |
| Fusicatenibacter saccharivorans | | | | | | | | | |
| Bacteria; Firmicutes; Clostridia; | 36 | 0.004956 | 0.944848 | 0.001907 | 0.007485 | 0.673647 | 0.001801 | 0.007718 | 0.461216 |
| Clostridiales; Peptostreptococcaceae; | | | | | | | | | |
| Romboutsia; Other | | | | | | | | | |
| Bacteria; Proteobacteria; | 37 | 0.004912 | 6.282996 | 0.003563 | 0.024830 | 0.661431 | 0.022385 | 0.072649 | 0.769392 |
| Gammaproteobacteria; | | | | | | | | | |
| Enterobacterales; | | | | | | | | | |
| Enterobacteriaceae; Other; Other | | | | | | | | | |
| Bacteria; Actinobacteria; | 38 | 0.004903 | 1.164344 | 0.006563 | 0.018823 | 0.834206 | 0.007641 | 0.027702 | 0.601677 |
| Coriobacteriia; Coriobacteriales; | | | | | | | | | |
| Coriobacteriaceae; Collinsella; | | | | | | | | | |
| Collinsella aerofaciens | | | | | | | | | |
| Bacteria; Proteobacteria; | 39 | 0.004898 | 0.518059 | 0.001464 | 0.007529 | 0.739965 | 0.000758 | 0.008773 | 0.452830 |
| Gammaproteobacteria; Pasteurellales; | | | | | | | | | |
| Pasteurellaceae; Haemophilus; | | | | | | | | | |
| Haemophilus parainfluenzae | | | | | | | | | |
| Bacteria; Firmicutes; Clostridia; | 40 | 0.004897 | 0.899522 | 0.001518 | 0.002170 | 0.855148 | 0.001365 | 0.003206 | 0.679245 |
| Clostridiales; Lachnospiraceae; | | | | | | | | | |
| Fusicatenibacter; Other | | | | | | | | | |
| Bacteria; Firmicutes; Clostridia; | 41 | 0.004871 | 1.170455 | 0.003749 | 0.012395 | 0.973822 | 0.004388 | 0.009337 | 0.882600 |
| Clostridiales; Lachnospiraceae; | | | | | | | | | |
| Anaerostipes; Other | | | | | | | | | |
| Bacteria; Firmicutes; | 42 | 0.004864 | 0.586039 | 0.000154 | 0.000807 | 0.544503 | 0.000090 | 0.000458 | 0.253669 |
| Erysipelotrichia; Erysipelotrichales; | | | | | | | | | |
| Erysipelotrichaceae; | | | | | | | | | |
| Turicibacter; Other | | | | | | | | | |
| Bacteria; Firmicutes; | 43 | 0.004844 | 0.876789 | 0.001875 | 0.002546 | 0.942408 | 0.001644 | 0.003814 | 0.863732 |
| Other; Other; Other; Other; Other | | | | | | | | | |
| Bacteria; Bacteroidetes; Bacteroidia; | 44 | 0.004739 | 0.680514 | 0.068297 | 0.095683 | 0.961606 | 0.046477 | 0.081902 | 0.897275 |
| Bacteroidales; Bacteroidaceae; | | | | | | | | | |
| Bacteroides; | | | | | | | | | |
| Bacteroides vulgatus | | | | | | | | | |
| Bacteria; Proteobacteria; | 45 | 0.004737 | 4.399993 | 0.000769 | 0.003514 | 0.521815 | 0.003383 | 0.009955 | 0.624738 |
| Gammaproteobacteria; Other; Other; | | | | | | | | | |
| Other; Other | | | | | | | | | |
| Bacteria; Bacteroidetes; Bacteroidia; | 46 | 0.004515 | 0.282668 | 0.000510 | 0.001284 | 0.544503 | 0.000144 | 0.000634 | 0.301887 |
| Bacteroidales; Porphyromonadaceae; | | | | | | | | | |
| Butyricimonas; Other | | | | | | | | | |
| Bacteria; Bacteroidetes; Bacteroidia; | 47 | 0.004514 | 0.426001 | 0.007366 | 0.024075 | 0.872600 | 0.003138 | 0.008754 | 0.733753 |
| Bacteroidales; Rikenellaceae; | | | | | | | | | |
| Alistipes; Other | | | | | | | | | |
| Bacteria; Firmicutes; Clostridia; | 48 | 0.004410 | 1.581438 | 0.019726 | 0.031062 | 0.994764 | 0.031195 | 0.061840 | 0.972746 |
| Clostridiales; Lachnospiraceae; | | | | | | | | | |
| Blautia; Other | | | | | | | | | |
| Bacteria; Firmicutes; Clostridia; | 49 | 0.004371 | 0.861389 | 0.062866 | 0.047874 | 0.996510 | 0.054152 | 0.054363 | 0.985325 |
| Clostridiales; Lachnospiraceae; | | | | | | | | | |
| Other; Other | | | | | | | | | |
| Bacteria; Firmicutes; Clostridia; | 50 | 0.004361 | 0.892065 | 0.005688 | 0.011516 | 0.780105 | 0.005074 | 0.020778 | 0.582809 |
| Clostridiales; Ruminococcaceae; | | | | | | | | | |
| Gemmiger; Gemmiger formicilis | | | | | | | | | |
| Bacteria; Firmicutes; Erysipelotrichia; | 51 | 0.004327 | 0.844106 | 0.000304 | 0.000939 | 0.521815 | 0.000257 | 0.000904 | 0.299790 |
| Erysipelotrichales; Erysipelotrichaceae; | | | | | | | | | |
| Erysipelatoclostridium; | | | | | | | | | |
| [Clostridium] spiroforme | | | | | | | | | |
| Bacteria; Bacteroidetes; Bacteroidia; | 52 | 0.004279 | 0.661422 | 0.034407 | 0.044854 | 0.945899 | 0.022757 | 0.044217 | 0.853249 |
| Bacteroidales; Bacteroidaceae; | | | | | | | | | |
| Bacteroides; | | | | | | | | | |
| Bacteroide suniformis | | | | | | | | | |
| Bacteria; Firmicutes; Clostridia; | 53 | 0.004264 | 1.102315 | 0.006553 | 0.009059 | 0.919721 | 0.007223 | 0.013643 | 0.788260 |
| Clostridiales; Lachnospiraceae; | | | | | | | | | |
| Dorea; Dorea longicatena | | | | | | | | | |
| Bacteria; Actinobacteria; | 54 | 0.004179 | 1.310923 | 0.020048 | 0.036905 | 0.956370 | 0.026282 | 0.066768 | 0.867925 |
| Actinobacteria; Bifidobacteriales; | | | | | | | | | |
| Bifidobacteriaceae; | | | | | | | | | |
| Bifidobacterium; Other | | | | | | | | | |
| Bacteria; Firmicutes; Clostridia; | 55 | 0.004168 | 7.940624 | 0.001148 | 0.009254 | 0.530541 | 0.009115 | 0.032504 | 0.570231 |
| Clostridiales; Lachnospiraceae; | | | | | | | | | |
| Blautia; | | | | | | | | | |
| Blautia caecimuris | | | | | | | | | |
| Bacteria; Bacteroidetes; Bacteroidia; | 56 | 0.004167 | 0.970509 | 0.101366 | 0.098052 | 0.986038 | 0.098377 | 0.108029 | 0.958071 |
| Bacteroidales; Bacteroidaceae; | | | | | | | | | |
| Bacteroides; Other | | | | | | | | | |
| Bacteria; Firmicutes; Clostridia; | 57 | 0.004124 | 1.126806 | 0.012282 | 0.021109 | 0.963351 | 0.013839 | 0.023652 | 0.844864 |
| Clostridiales; Lachnospiraceae; | | | | | | | | | |
| Anaerostipes; | | | | | | | | | |
| Anaerostipes hadrus | | | | | | | | | |
| Bacteria; Firmicutes; Clostridia; | 58 | 0.003973 | 2.206604 | 0.001696 | 0.003399 | 0.902269 | 0.003742 | 0.012570 | 0.807128 |
| Clostridiales; Lachnospiraceae; | | | | | | | | | |
| Ruminococcus2; Other | | | | | | | | | |
| Bacteria; Bacteroidetes; Bacteroidia; | 59 | 0.003972 | 0.932920 | 0.004877 | 0.008018 | 0.916230 | 0.004550 | 0.011480 | 0.763103 |
| Bacteroidales; Porphyromonadaceae; | | | | | | | | | |
| Parabacteroides; Other | | | | | | | | | |
| Bacteria; Firmicutes; Negativicutes; | 60 | 0.003915 | 1.457596 | 0.005449 | 0.014591 | 0.650960 | 0.007942 | 0.019553 | 0.637317 |
| Veillonellales; Veillonellaceae; | | | | | | | | | |
| Dialister; Dialister invisus | | | | | | | | | |
| Bacteria; Other; Other; Other; | 61 | 0.003783 | 0.505194 | 0.026969 | 0.043756 | 0.954625 | 0.013624 | 0.027468 | 0.920335 |
| Other; Other; Other | | | | | | | | | |
| Bacteria; Firmicutes; Clostridia; | 62 | 0.003746 | 1.096624 | 0.001056 | 0.002752 | 0.884817 | 0.001158 | 0.003329 | 0.706499 |
| Clostridiales; Peptostreptococcaceae; | | | | | | | | | |
| Other; Other | | | | | | | | | |
| Bacteria; Firmicutes; Clostridia; | 63 | 0.003738 | 1.122557 | 0.013301 | 0.017704 | 0.989529 | 0.014931 | 0.026591 | 0.964361 |
| Clostridiales; Lachnospiraceae; | | | | | | | | | |
| Lachnospiracea_incertae_sedis; Other | | | | | | | | | |
| Bacteria; Bacteroidetes; Bacteroidia; | 64 | 0.003692 | 0.556329 | 0.007016 | 0.022595 | 0.774869 | 0.003903 | 0.014320 | 0.626834 |
| Bacteroidales; Rikenellaceae; | | | | | | | | | |
| Alistipes; Alistipes onderdonkii | | | | | | | | | |
| Bacteria; Bacteroidetes; Bacteroidia; | 65 | 0.003691 | 0.440683 | 0.001199 | 0.002404 | 0.766143 | 0.000529 | 0.001052 | 0.517820 |
| Bacteroidales; Odoribacteraceae; | | | | | | | | | |
| Odoribacter; Odoribacter splanchnicus | | | | | | | | | |
| Bacteria; Firmicutes; Clostridia; | 66 | 0.003686 | 0.913324 | 0.000561 | 0.001490 | 0.582897 | 0.000512 | 0.002143 | 0.327044 |
| Clostridiales; Ruminococcaceae; | | | | | | | | | |
| Gemmiger; Other | | | | | | | | | |
| Bacteria; Firmicutes; | 67 | 0.003676 | 0.185996 | 0.000649 | 0.006125 | 0.520070 | 0.000121 | 0.000810 | 0.276730 |
| Erysipelotrichia; Erysipelotrichales; | | | | | | | | | |
| Erysipelotrichaceae; Other; Other | | | | | | | | | |
| Bacteria; Firmicutes; Clostridia; | 68 | 0.003673 | 0.217265 | 0.000049 | 0.000144 | 0.380454 | 0.000011 | 0.000049 | 0.117400 |
| Clostridiales; Clostridiales | | | | | | | | | |
| Family XIII. Incertae Sedis; | | | | | | | | | |
| Ihubacter; Ihubacter massiliensis | | | | | | | | | |
| Bacteria; Bacteroidetes; | 69 | 0.003672 | 0.580127 | 0.007912 | 0.016860 | 0.858639 | 0.004590 | 0.013305 | 0.704403 |
| Bacteroidia; Bacteroidales; | | | | | | | | | |
| Bacteroidaceae; Bacteroides; | | | | | | | | | |
| Bacteroides caccae | | | | | | | | | |
| Bacteria; Firmicutes; Clostridia; | 70 | 0.003665 | 0.448301 | 0.000467 | 0.001038 | 0.565445 | 0.000209 | 0.000857 | 0.341719 |
| Clostridiales; Ruminococcaceae; | | | | | | | | | |
| Intestinimonas; Other | | | | | | | | | |
| Bacteria; Firmicutes; Clostridia; | 71 | 0.003657 | 3.249400 | 0.000111 | 0.000445 | 0.483421 | 0.000359 | 0.001053 | 0.534591 |
| Clostridiales; Lachnospiraceae; | | | | | | | | | |
| Lachnoclostridium; | | | | | | | | | |
| [Clostridium] aldenense | | | | | | | | | |
| Bacteria; Firmicutes; Clostridia; | 72 | 0.003640 | 0.331898 | 0.000546 | 0.001248 | 0.333333 | 0.000181 | 0.000969 | 0.174004 |
| Clostridiales; Lachnospiraceae; | | | | | | | | | |
| Coprococcus; Coprococcus catus | | | | | | | | | |
| Bacteria; Bacteroidetes; Bacteroidia; | 73 | 0.003625 | 0.765360 | 0.007358 | 0.023955 | 0.806283 | 0.005632 | 0.017336 | 0.668763 |
| Bacteroidales; Tannerellaceae; | | | | | | | | | |
| Parabacteroides; | | | | | | | | | |
| Parabacteroides distasonis | | | | | | | | | |
| Bacteria; Firmicutes; Clostridia; | 74 | 0.003556 | 0.256160 | 0.000067 | 0.000156 | 0.485166 | 0.000017 | 0.000079 | 0.186583 |
| Clostridiales; Ruminococcaceae; | | | | | | | | | |
| Pseudoflavonifractor; | | | | | | | | | |
| Pseudoflavonifractor capillosus | | | | | | | | | |
| Bacteria; Firmicutes; Clostridia; | 75 | 0.003548 | 0.213138 | 0.001499 | 0.004240 | 0.507853 | 0.000320 | 0.001300 | 0.243187 |
| Clostridiales; Ruminococcaceae; | | | | | | | | | |
| Ruminococcus; | | | | | | | | | |
| Ruminococcus callidus | | | | | | | | | |
| Bacteria; Fusobacteria; Fusobacteriia; | 76 | 0.003532 | 39.574321 | 0.000137 | 0.001627 | 0.247818 | 0.005410 | 0.027960 | 0.385744 |
| Fusobacteriales; Fusobacteriaceae; | | | | | | | | | |
| Fusobacterium; Other | | | | | | | | | |
| Bacteria; Proteobacteria; | 77 | 0.003513 | 4.627845 | 0.007603 | 0.034601 | 0.530541 | 0.035184 | 0.110490 | 0.547170 |
| Gammaproteobacteria; Enterobacterales; | | | | | | | | | |
| Other; Other; Other | | | | | | | | | |
| Bacteria; Bacteroidetes; Bacteroidia; | 78 | 0.003466 | 0.451673 | 0.007151 | 0.011051 | 0.792321 | 0.003230 | 0.007353 | 0.635220 |
| Bacteroidales; Rikenellaceae; | | | | | | | | | |
| Alistipes; Alistipes putredinis | | | | | | | | | |
| Bacteria; Firmicutes; Clostridia; | 79 | 0.003367 | 0.534609 | 0.000043 | 0.000082 | 0.589878 | 0.000023 | 0.000071 | 0.400419 |
| Other; Other; Other; Other | | | | | | | | | |
| Bacteria; Firmicutes; Clostridia; | 80 | 0.003294 | 2.150444 | 0.000204 | 0.001014 | 0.593368 | 0.000439 | 0.001235 | 0.482180 |
| Clostridiales; Lachnospiraceae; | | | | | | | | | |
| Lachnoclostridium; [Clostridium] | | | | | | | | | |
| glycyrrhizinilyticum | | | | | | | | | |
| Bacteria; Firmicutes; Clostridia; | 81 | 0.003279 | 0.725527 | 0.000057 | 0.000204 | 0.462478 | 0.000041 | 0.000195 | 0.209644 |
| Clostridiales; Not Available; | | | | | | | | | |
| Intestinimonas; | | | | | | | | | |
| Intestinimonas butyriciproducens | | | | | | | | | |
| Bacteria; Firmicutes; Clostridia; | 82 | 0.003270 | 0.947557 | 0.003180 | 0.008982 | 0.766143 | 0.003013 | 0.010184 | 0.597484 |
| Clostridiales; Ruminococcaceae; | | | | | | | | | |
| Ruminococcus; | | | | | | | | | |
| Ruminococcus faecis | | | | | | | | | |
| Bacteria; Firmicutes; Clostridia; | 83 | 0.003242 | 0.626182 | 0.002427 | 0.006841 | 0.413613 | 0.001520 | 0.010646 | 0.257862 |
| Clostridiales; Lachnospiraceae; | | | | | | | | | |
| Roseburia; Roseburia faecis | | | | | | | | | |
| Bacteria; Firmicutes; Clostridia; | 84 | 0.003234 | 1.218792 | 0.000396 | 0.001292 | 0.815009 | 0.000482 | 0.002604 | 0.670860 |
| Clostridiales; Ruminococcaceae; | | | | | | | | | |
| Flavonifractor; Other | | | | | | | | | |
| Bacteria; Firmicutes; Bacilli; | 85 | 0.003186 | 1.563053 | 0.010593 | 0.027852 | 0.996510 | 0.016557 | 0.041320 | 0.966457 |
| Lactobacillales; Streptococcaceae; | | | | | | | | | |
| Streptococcus; Other | | | | | | | | | |
| Bacteria; Bacteroidetes; Bacteroidia; | 86 | 0.003178 | 0.334760 | 0.001568 | 0.003304 | 0.657941 | 0.000525 | 0.001528 | 0.452830 |
| Bacteroidales; Rikenellaceae; | | | | | | | | | |
| Alistipes; Alistipes shahii | | | | | | | | | |
| Bacteria; Firmicutes; Clostridia; | 87 | 0.003173 | 1.158885 | 0.001138 | 0.003845 | 0.886562 | 0.001319 | 0.003364 | 0.756813 |
| Clostridiales; Ruminococcaceae; | | | | | | | | | |
| Flavonifractor; | | | | | | | | | |
| Flavonifractor plautii | | | | | | | | | |
| Bacteria; Firmicutes; Clostridia; | 88 | 0.003165 | 0.775393 | 0.000399 | 0.002169 | 0.539267 | 0.000309 | 0.001143 | 0.339623 |
| Clostridiales; Eubacteriaceae; | | | | | | | | | |
| Eubacterium; Other | | | | | | | | | |
| Bacteria; Firmicutes; Clostridia; | 89 | 0.003147 | 0.870148 | 0.001917 | 0.008278 | 0.783595 | 0.001668 | 0.017166 | 0.555556 |
| Clostridiales; Clostridiaceae; | | | | | | | | | |
| Clostridium; Other | | | | | | | | | |
| Bacteria; Firmicutes; Erysipelotrichia; | 90 | 0.003146 | 0.421941 | 0.000216 | 0.001993 | 0.408377 | 0.000091 | 0.000704 | 0.169811 |
| Erysipelotrichales; Erysipelotrichaceae; | | | | | | | | | |
| Massilimicrobiota; | | | | | | | | | |
| Massilimicrobiota timonensis | | | | | | | | | |
| Bacteria; Actinobacteria; | 91 | 0.003125 | 0.324292 | 0.016442 | 0.046861 | 0.406632 | 0.005332 | 0.023558 | 0.366876 |
| Actinobacteria; Bifidobacteriales; | | | | | | | | | |
| Bifidobacteriaceae; Bifidobacterium; | | | | | | | | | |
| Bifidobacterium adolescentis | | | | | | | | | |
| Archaea; Euryarchaeota; Methanobacteria; | 92 | 0.003113 | 0.139399 | 0.001241 | 0.011593 | 0.361257 | 0.000173 | 0.001805 | 0.146751 |
| Methanobacteriales; Methanobacteriaceae; | | | | | | | | | |
| Methanobrevibacter; Other | | | | | | | | | |
| Bacteria; Firmicutes; Clostridia; | 93 | 0.003103 | 1.398687 | 0.000307 | 0.001139 | 0.687609 | 0.000429 | 0.001476 | 0.595388 |
| Clostridiales; Peptostreptococcaceae; | | | | | | | | | |
| Intestinibacter; Other | | | | | | | | | |
| Bacteria; Actinobacteria; | 94 | 0.003044 | 1.207493 | 0.000536 | 0.002138 | 0.687609 | 0.000647 | 0.001878 | 0.568134 |
| Coriobacteriia; Eggerthellales; | | | | | | | | | |
| Eggerthellaceae; | | | | | | | | | |
| Eggerthella; | | | | | | | | | |
| Eggerthella lenta | | | | | | | | | |
| Bacteria; Firmicutes; Clostridia; | 95 | 0.003018 | 0.828759 | 0.000042 | 0.000161 | 0.507853 | 0.000034 | 0.000163 | 0.295597 |
| Clostridiales; Ruminococcaceae; | | | | | | | | | |
| Anaerotruncus; | | | | | | | | | |
| Anaerotruncus colihominis | | | | | | | | | |
| Bacteria; Firmicutes; Negativicutes; | 96 | 0.002994 | 0.674143 | 0.003059 | 0.010139 | 0.670157 | 0.002062 | 0.005998 | 0.582809 |
| Selenomonadales; Veillonellaceae; | | | | | | | | | |
| Dialister; Other | | | | | | | | | |
| Bacteria; Bacteroidetes; Bacteroidia; | 97 | 0.002985 | 0.547289 | 0.000237 | 0.000578 | 0.617801 | 0.000130 | 0.000592 | 0.383648 |
| Bacteroidales; Porphyromonadaceae; | | | | | | | | | |
| Odoribacter; Other | | | | | | | | | |
| Bacteria; Fusobacteria; | 98 | 0.002981 | 16.117410 | 0.000206 | 0.002672 | 0.172775 | 0.003326 | 0.018044 | 0.371069 |
| Fusobacteriia; Fusobacteriales; | | | | | | | | | |
| Fusobacteriaceae; Fusobacterium; | | | | | | | | | |
| Fusobacterium nucleatum | | | | | | | | | |
| Bacteria; Firmicutes; Clostridia; | 99 | 0.002916 | 0.589134 | 0.000243 | 0.002463 | 0.527051 | 0.000143 | 0.000973 | 0.243187 |
| Clostridiales; Lachnospiraceae; | | | | | | | | | |
| Eisenbergiella; | | | | | | | | | |
| Eisenbergiella tayi | | | | | | | | | |
| Bacteria; Actinobacteria; | 100 | 0.002882 | 0.727827 | 0.004403 | 0.012714 | 0.762653 | 0.003205 | 0.009131 | 0.584906 |
| Actinobacteria; Coriobacteriales; | | | | | | | | | |
| Coriobacteriaceae; | | | | | | | | | |
| Collinsella; Other | | | | | | | | | |

IBD = Inflammatory Bowel Disease; CD = Crohn's Disease; UC = Ulcerative Colitis; Ctrl = Control; RFFR = Random Forest Feature Rank; # = Rank; MDA = Mean Decrease Accuracy; FC = Fold Change; M = Mean; SD = Standard Deviation; DP = Detection Prevalence;

TABLE 16
Pediatric CDI vs Pediatric Control delineation features - (taxonomy)

| Feature | | | FC | | | | | | |
|---|---|---|---|---|---|---|---|---|---|
| (Taxonomic order: Kingdom; | | | CDI | | | | | | |
| Phylum; Class; Order; | RFFR | | vs | Abundance (P-CDI) | | | Abundance (P-Ctrl) | | |
| Family; Genus) | # | MDA | Ctrl | M | SD | DP | M | SD | DP |
| Bacteria; Firmicutes; Clostridia; | 1 | 0.050880 | 0.211185 | 0.009346 | 0.037604 | 0.337423 | 0.044255 | 0.047610 | 0.989305 |
| Clostridiales; Ruminococcaceae; | | | | | | | | | |
| Faecalibacterium | | | | | | | | | |
| Bacteria; Firmicutes; Clostridia; | 2 | 0.046493 | 0.380728 | 0.007797 | 0.024579 | 0.472393 | 0.020480 | 0.021792 | 0.994652 |
| Clostridiales; Ruminococcaceae; | | | | | | | | | |
| Other | | | | | | | | | |
| Bacteria; Bacteroidetes; Bacteroidia; | 3 | 0.046428 | 0.161007 | 0.003392 | 0.013174 | 0.306748 | 0.021069 | 0.039105 | 0.973262 |
| Bacteroidales; Porphyromonadaceae; | | | | | | | | | |
| Parabacteroides | | | | | | | | | |
| Bacteria; Bacteroidetes; Bacteroidia; | 4 | 0.044322 | 0.132454 | 0.010516 | 0.078929 | 0.300613 | 0.079396 | 0.073106 | 0.973262 |
| Bacteroidales; Rikenellaceae; | | | | | | | | | |
| Alistipes | | | | | | | | | |
| Bacteria; Firmicutes; Clostridia; | 5 | 0.041510 | 0.076145 | 0.001182 | 0.006649 | 0.177914 | 0.015519 | 0.022756 | 0.935829 |
| Clostridiales; Lachnospiraceae; | | | | | | | | | |
| [Eubacterium] rectale group | | | | | | | | | |
| Bacteria; Firmicutes; Clostridia; | 6 | 0.038082 | 0.063152 | 0.000387 | 0.002200 | 0.122699 | 0.006126 | 0.009801 | 0.903743 |
| Clostridiales; Lachnospiraceae; | | | | | | | | | |
| Coprococcus | | | | | | | | | |
| Bacteria; Firmicutes; Clostridia; | 7 | 0.035840 | 0.151565 | 0.001147 | 0.006662 | 0.245399 | 0.007567 | 0.009631 | 0.962567 |
| Clostridiales; Lachnospiraceae; | | | | | | | | | |
| Roseburia | | | | | | | | | |
| Bacteria; Firmicutes; Bacilli; | 8 | 0.035642 | 842.982629 | 0.007133 | 0.018683 | 0.760736 | 0.000008 | 0.000087 | 0.032086 |
| Lactobacillales; Other; Other | | | | | | | | | |
| Bacteria; Firmicutes; Clostridia; | 9 | 0.035317 | 0.812626 | 0.004717 | 0.031952 | 0.177914 | 0.005805 | 0.008967 | 0.946524 |
| Clostridiales; Lachnospiraceae; | | | | | | | | | |
| Dorea | | | | | | | | | |
| Bacteria; Firmicutes; Clostridia; | 10 | 0.032443 | 1.106990 | 0.004086 | 0.015414 | 0.202454 | 0.003691 | 0.003880 | 0.962567 |
| Clostridiales; Lachnospiraceae; | | | | | | | | | |
| Fusicatenibacter | | | | | | | | | |
| Bacteria; Bacteroidetes; Bacteroidia; | 11 | 0.030484 | 0.220839 | 0.094242 | 0.201046 | 0.779141 | 0.426747 | 0.201042 | 0.989305 |
| Bacteroidales; Bacteroidaceae; | | | | | | | | | |
| Bacteroides | | | | | | | | | |
| Bacteria; Bacteroidetes; Bacteroidia; | 12 | 0.028713 | 0.001090 | 0.000005 | 0.000042 | 0.042945 | 0.004637 | 0.007784 | 0.737968 |
| Bacteroidales; Porphyromon | | | | | | | | | |
| adaceae; Odoribacter | | | | | | | | | |
| Bacteria; Firmicutes; Clostridia; | 13 | 0.027362 | 0.666573 | 0.001482 | 0.007581 | 0.208589 | 0.002224 | 0.002765 | 0.914439 |
| Clostridiales; Lachnospiraceae; | | | | | | | | | |
| Ruminococcus2 | | | | | | | | | |
| Bacteria; Firmicutes; Clostridia; | 14 | 0.025846 | #DIV/0! | 0.007821 | 0.031595 | 0.693252 | 0.000000 | 0.000000 | 0.000000 |
| Clostridiales; Peptostreptococcaceae; | | | | | | | | | |
| Clostridioides | | | | | | | | | |
| Bacteria; Firmicutes; Bacilli; | 15 | 0.025100 | 181.461752 | 0.110940 | 0.208194 | 0.791411 | 0.000611 | 0.005278 | 0.074866 |
| Lactobacillales; Enterococcaceae; | | | | | | | | | |
| Enterococcus | | | | | | | | | |
| Bacteria; Firmicutes; Negativicutes; | 16 | 0.023294 | 0.036064 | 0.001595 | 0.012929 | 0.171779 | 0.044240 | 0.074564 | 0.855615 |
| Selenomonadales; Veillonellaceae; | | | | | | | | | |
| Dialister | | | | | | | | | |
| Bacteria; Firmicutes; Clostridia; | 17 | 0.020923 | 1.049341 | 0.012709 | 0.032446 | 0.509202 | 0.012112 | 0.011581 | 0.983957 |
| Clostridiales; Lachnospiraceae; | | | | | | | | | |
| Lachnospiracea_{—}incertae_{—}sedis | | | | | | | | | |
| Bacteria; Bacteroidetes; Bacteroidia; | 18 | 0.018514 | 0.484277 | 0.007163 | 0.038013 | 0.374233 | 0.014791 | 0.022495 | 0.887701 |
| Bacteroidales; Tannerellaceae; | | | | | | | | | |
| Parabacteroides | | | | | | | | | |
| Bacteria; Actinobacteria; | 19 | 0.016877 | 69.303670 | 0.006208 | 0.014624 | 0.582822 | 0.000090 | 0.000324 | 0.165775 |
| Coriobacteriia; Eggerthellales; | | | | | | | | | |
| Eggerthellaceae; Eggerthella | | | | | | | | | |
| Bacteria; Firmicutes; Negativicutes; | 20 | 0.016254 | 0.124109 | 0.004752 | 0.031692 | 0.220859 | 0.038293 | 0.062985 | 0.850267 |
| Veillonellales; Veillonellaceae; | | | | | | | | | |
| Dialister | | | | | | | | | |
| Bacteria; Firmicutes; Erysipelotrichia; | 21 | 0.016027 | 68.095014 | 0.032024 | 0.068740 | 0.809816 | 0.000470 | 0.000938 | 0.524064 |
| Erysipelotrichales; | | | | | | | | | |
| Erysipelotrichaceae; | | | | | | | | | |
| Erysipelatoclostridium | | | | | | | | | |
| Bacteria; Firmicutes; Clostridia; | 22 | 0.015604 | 0.038942 | 0.000123 | 0.001172 | 0.116564 | 0.003148 | 0.006829 | 0.802139 |
| Clostridiales; Ruminococcaceae; | | | | | | | | | |
| Clostridium IV | | | | | | | | | |
| Bacteria; Firmicutes; Clostridia; | 23 | 0.015165 | 0.401895 | 0.012298 | 0.026170 | 0.791411 | 0.030600 | 0.044063 | 1.000000 |
| Clostridiales; Lachnospiraceae; | | | | | | | | | |
| Other | | | | | | | | | |
| Bacteria; Firmicutes; Clostridia; | 24 | 0.015099 | 46.107430 | 0.015279 | 0.045057 | 0.711656 | 0.000331 | 0.000737 | 0.449198 |
| Clostridiales; Lachnospiraceae; | | | | | | | | | |
| Lachnoclostridium | | | | | | | | | |
| Bacteria; Firmicutes; Clostridia; | 25 | 0.013587 | 0.250701 | 0.004879 | 0.018782 | 0.269939 | 0.019461 | 0.046161 | 0.871658 |
| Clostridiales; Ruminococcaceae; | | | | | | | | | |
| Ruminococcus | | | | | | | | | |
| Bacteria; Firmicutes; Clostridia; | 26 | 0.012836 | 0.719791 | 0.005329 | 0.043906 | 0.171779 | 0.007404 | 0.014245 | 0.754011 |
| Clostridiales; Ruminococcaceae; | | | | | | | | | |
| Gemmiger | | | | | | | | | |
| Bacteria; Bacteroidetes; Bacteroidia; | 27 | 0.012553 | 0.256510 | 0.001120 | 0.006767 | 0.141104 | 0.004364 | 0.007279 | 0.759358 |
| Bacteroidales; Odoribacteraceae; | | | | | | | | | |
| Odoribacter | | | | | | | | | |
| Bacteria; Firmicutes; Bacilli; Other; | 28 | 0.011115 | 1597.768886 | 0.000398 | 0.000891 | 0.503067 | 0.000000 | 0.000003 | 0.005348 |
| Other; Other | | | | | | | | | |
| Bacteria; Bacteroidetes; Bacteroidia; | 29 | 0.008453 | 0.038567 | 0.000023 | 0.000204 | 0.110429 | 0.000587 | 0.002368 | 0.524064 |
| Bacteroidales; Other; Other | | | | | | | | | |
| Bacteria; Actinobacteria; | 30 | 0.008310 | 0.036855 | 0.000050 | 0.000573 | 0.061350 | 0.001367 | 0.003301 | 0.545455 |
| Actinobacteria; Coriobacteriales; | | | | | | | | | |
| Coriobacteriaceae; Collinsella | | | | | | | | | |
| Bacteria; Proteobacteria; Gamma | 31 | 0.008145 | 35.248715 | 0.049688 | 0.127352 | 0.730061 | 0.001410 | 0.012707 | 0.278075 |
| proteobacteria; Enterobacterales; | | | | | | | | | |
| Enterobacteriaceae; Escherichia | | | | | | | | | |
| Bacteria; Firmicutes; Bacilli; | 32 | 0.007536 | #DIV/0! | 0.004390 | 0.026302 | 0.509202 | 0.000000 | 0.000000 | 0.000000 |
| Lactobacillales; Enterococcaceae; | | | | | | | | | |
| Other | | | | | | | | | |
| Bacteria; Actinobacteria; | 33 | 0.007293 | 0.091814 | 0.000043 | 0.000440 | 0.036810 | 0.000463 | 0.001174 | 0.491979 |
| Actinobacteria; Coriobacteriales; | | | | | | | | | |
| Coriobacteriaceae; Other | | | | | | | | | |
| Bacteria; Proteobacteria; Beta- | 34 | 0.007185 | 0.331350 | 0.001844 | 0.009368 | 0.171779 | 0.005565 | 0.015103 | 0.663102 |
| proteobacteria;Burkholderiales; | | | | | | | | | |
| Sutterellaceae; Parasutterella | | | | | | | | | |
| Bacteria; Proteobacteria; Gamma | 35 | 0.007121 | 11.015887 | 0.045087 | 0.151893 | 0.840491 | 0.004093 | 0.023097 | 0.315508 |
| proteobacteria; Enterobacteriales; | | | | | | | | | |
| Enterobacteriaceae; Other | | | | | | | | | |
| Bacteria; Verrucomicrobia; | 36 | 0.007052 | 0.446210 | 0.011846 | 0.083730 | 0.226994 | 0.026547 | 0.072308 | 0.711230 |
| Verrucomicrobiae; | | | | | | | | | |
| Verrucomicrobiales; | | | | | | | | | |
| Verrucomicrobiaceae; Akkermansia | | | | | | | | | |
| Bacteria; Firmicutes; Clostridia; | 37 | 0.006994 | 4.046665 | 0.091226 | 0.150407 | 0.889571 | 0.022543 | 0.027331 | 0.994652 |
| Clostridiales; Lachnospiraceae; | | | | | | | | | |
| Blautia | | | | | | | | | |
| Bacteria; Firmicutes; Bacilli; | 38 | 0.006639 | 17.138984 | 0.073805 | 0.133505 | 0.944785 | 0.004306 | 0.010976 | 0.860963 |
| Lactobacillales; Streptococcaceae; | | | | | | | | | |
| Streptococcus | | | | | | | | | |
| Bacteria; Actinobacteria; | 39 | 0.006356 | 112.944476 | 0.006408 | 0.030049 | 0.613497 | 0.000057 | 0.000175 | 0.192513 |
| Actinobacteria; Actinomycetales; | | | | | | | | | |
| Actinomycetaceae; Schaalia | | | | | | | | | |
| Bacteria; Firmicutes; Clostridia; | 40 | 0.006337 | 4.552891 | 0.020449 | 0.063547 | 0.619632 | 0.004492 | 0.007820 | 0.925134 |
| Clostridiales; Lachnospiraceae; | | | | | | | | | |
| Anaerostipes | | | | | | | | | |
| Bacteria; Firmicutes; Clostridia; | 41 | 0.006095 | 0.604950 | 0.005139 | 0.016770 | 0.693252 | 0.008495 | 0.016000 | 0.978610 |
| Clostridiales; Other; Other | | | | | | | | | |
| Bacteria; Firmicutes; Clostridia; | 42 | 0.004653 | 102.734823 | 0.005096 | 0.026154 | 0.417178 | 0.000050 | 0.000214 | 0.117647 |
| Clostridiales; Clostridiaceae; | | | | | | | | | |
| Hungatella | | | | | | | | | |
| Bacteria; Firmicutes; Clostridia; | 43 | 0.004264 | 0.441805 | 0.000411 | 0.002207 | 0.061350 | 0.000930 | 0.002675 | 0.529412 |
| Clostridiales; Ruminococcaceae; | | | | | | | | | |
| Agathobaculum | | | | | | | | | |
| Bacteria; Firmicutes; Clostridia; | 44 | 0.003724 | 17.428965 | 0.012477 | 0.035348 | 0.638037 | 0.000716 | 0.001967 | 0.459893 |
| Clostridiales; Clostridiaceae; | | | | | | | | | |
| Clostridium | | | | | | | | | |
| Bacteria; Actinobacteria; | 45 | 0.003601 | 267.323021 | 0.000941 | 0.006184 | 0.355828 | 0.000004 | 0.000033 | 0.016043 |
| Actinobacteria; Corynebacteriales; | | | | | | | | | |
| Corynebacteriaceae; | | | | | | | | | |
| Corynebacterium | | | | | | | | | |
| Bacteria; Firmicutes; Negativicutes; | 46 | 0.003506 | 22.237996 | 0.014643 | 0.045727 | 0.705521 | 0.000658 | 0.001406 | 0.513369 |
| Veillonellales; Veillonellaceae; | | | | | | | | | |
| Veillonella | | | | | | | | | |
| Bacteria; Actinobacteria; | 47 | 0.003324 | 38.160755 | 0.004754 | 0.018003 | 0.644172 | 0.000125 | 0.000392 | 0.294118 |
| Actinobacteria; Actinomycetales; | | | | | | | | | |
| Actinomycetaceae; Actinomyces | | | | | | | | | |
| Bacteria; Firmicutes; Clostridia; | 48 | 0.002826 | 0.179629 | 0.000234 | 0.001024 | 0.196319 | 0.001302 | 0.003644 | 0.657754 |
| Clostridiales; Ruminococcaceae; | | | | | | | | | |
| [Clostridium] leptum group | | | | | | | | | |
| Bacteria; Fusobacteria; Fusobacteriia; | 49 | 0.002721 | 604.269814 | 0.002345 | 0.012720 | 0.269939 | 0.000004 | 0.000042 | 0.010695 |
| Fusobacteriales; Fusobacteriaceae; | | | | | | | | | |
| Fusobacterium | | | | | | | | | |
| Bacteria; Firmicutes; Erysipelotrichia; | 50 | 0.002687 | 65.046792 | 0.007263 | 0.039105 | 0.214724 | 0.000112 | 0.000571 | 0.101604 |
| Erysipelotrichales; | | | | | | | | | |
| Erysipelotrichaceae; | | | | | | | | | |
| Longicatena | | | | | | | | | |
| Bacteria; Firmicutes; Clostridia; | 51 | 0.002184 | 16.416961 | 0.005463 | 0.025266 | 0.607362 | 0.000333 | 0.000679 | 0.475936 |
| Clostridiales; Lachnospiraceae; | | | | | | | | | |
| Clostridium XIVa | | | | | | | | | |
| Bacteria; Firmicutes; Clostridia; | 52 | 0.002127 | 58.218456 | 0.006237 | 0.029087 | 0.312883 | 0.000107 | 0.000453 | 0.192513 |
| Clostridiales; Lachnospiraceae; | | | | | | | | | |
| Eisenbergiella | | | | | | | | | |
| Bacteria; Firmicutes; Bacilli; | 53 | 0.002034 | 3.492105 | 0.036792 | 0.134244 | 0.754601 | 0.010536 | 0.043024 | 0.470588 |
| Lactobacillales; Lactobacillus; | | | | | | | | | |
| Lactobacillus | | | | | | | | | |
| Bacteria; Firmicutes; Clostridia; | 54 | 0.002029 | 10.675751 | 0.012375 | 0.031853 | 0.625767 | 0.001159 | 0.003078 | 0.609626 |
| Clostridiales; Peptostreptococcaceae; | | | | | | | | | |
| Intestinibacter | | | | | | | | | |
| Bacteria; Proteobacteria; Gamma | 55 | 0.002012 | 1804.472700 | 0.012123 | 0.075931 | 0.368098 | 0.000007 | 0.000040 | 0.037433 |
| proteobacteria; Enterobacterales; | | | | | | | | | |
| Enterobacteriaceae; | | | | | | | | | |
| Citrobacter | | | | | | | | | |
| Bacteria; Firmicutes; Clostridia; | 56 | 0.001976 | 4.588196 | 0.007839 | 0.041596 | 0.349693 | 0.001709 | 0.003331 | 0.770053 |
| Clostridiales; Eubacteriaceae; | | | | | | | | | |
| Eubacterium | | | | | | | | | |
| Bacteria; Firmicutes; Tissierellia; | 57 | 0.001962 | 1208.452523 | 0.001809 | 0.016334 | 0.226994 | 0.000001 | 0.000015 | 0.010695 |
| Tissierellales; Peptoniphilaceae; | | | | | | | | | |
| Finegoldia | | | | | | | | | |
| Bacteria; Firmicutes; Bacilli; | 58 | 0.001960 | 26.900915 | 0.001663 | 0.005044 | 0.552147 | 0.000062 | 0.000144 | 0.245989 |
| Lactobacillales; Carnobacteriaceae; | | | | | | | | | |
| Granulicatella | | | | | | | | | |
| Bacteria; Proteobacteria; Gamma | 59 | 0.001871 | 0.000000 | 0.000000 | 0.000000 | 0.000000 | 0.000033 | 0.000323 | 0.021390 |
| proteobacteria; Enterobacterales; | | | | | | | | | |
| Enterobacteriaceae; Shimwellia | | | | | | | | | |
| Bacteria; Firmicutes; Negativicutes; | 60 | 0.001847 | 0.029768 | 0.000062 | 0.00030 | 0.079755 | 0.002093 | 0.006544 | 0.262032 |
| Selenomonadales; | | | | | | | | | |
| Acidaminococcaceae; | | | | | | | | | |
| Phascolarctobacterium | | | | | | | | | |
| Bacteria; Firmicutes; Bacilli; | 61 | 0.001813 | 0.177072 | 0.000428 | 0.003028 | 0.251534 | 0.002417 | 0.019002 | 0.128342 |
| Bacillales; Staphylococcaceae; | | | | | | | | | |
| Staphylococcus | | | | | | | | | |
| Bacteria; Actinobacteria; | 62 | 0.001787 | 6.029397 | 0.000179 | 0.000538 | 0.282209 | 0.000030 | 0.000119 | 0.096257 |
| Actinobacteria; Coriobacteriales; | | | | | | | | | |
| Coriobacteriaceae; Eggerthella | | | | | | | | | |
| Bacteria; Proteobacteria; Beta- | 63 | 0.001776 | 0.733855 | 0.002627 | 0.028696 | 0.110429 | 0.003579 | 0.012621 | 0.497326 |
| proteobacteria; Burkholderiales; | | | | | | | | | |
| Sutterellaceae; Sutterella | | | | | | | | | |
| Bacteria; Firmicutes; Clostridia; | 64 | 0.001762 | 0.000000 | 0.000000 | 0.000000 | 0.000000 | 0.000107 | 0.000304 | 0.251337 |
| Clostridiales; Lachnospiraceae; | | | | | | | | | |
| Anaerobutyricum | | | | | | | | | |
| Bacteria; Firmicutes; Clostridia; | 65 | 0.001728 | 569.534883 | 0.001567 | 0.010485 | 0.239264 | 0.000003 | 0.000024 | 0.016043 |
| Clostridiales; Peptostreptococcaceae; | | | | | | | | | |
| Peptostreptococcus | | | | | | | | | |
| Bacteria; Firmicutes; Erysipelotrichia; | 66 | 0.001727 | 0.119789 | 0.000042 | 0.000214 | 0.085890 | 0.000347 | 0.000870 | 0.422460 |
| Erysipelotrichales; | | | | | | | | | |
| Erysipelotrichaceae; | | | | | | | | | |
| Holdemania | | | | | | | | | |
| Bacteria; Firmicutes; Clostridia; | 67 | 0.001622 | 660.751733 | 0.000755 | 0.003002 | 0.208589 | 0.000001 | 0.000009 | 0.016043 |
| Clostridiales; Sellimonas; | | | | | | | | | |
| Drancourtella | | | | | | | | | |
| massiliensis | | | | | | | | | |
| Bacteria; Firmicutes; Clostridia; | 68 | 0.001617 | 0.166528 | 0.000167 | 0.000839 | 0.085890 | 0.001004 | 0.002606 | 0.385027 |
| Clostridiales; Not | | | | | | | | | |
| Available; Monoglobus | | | | | | | | | |
| Bacteria; Firmicutes; Clostridia; | 69 | 0.001614 | 1.481333 | 0.002166 | 0.008636 | 0.484663 | 0.001462 | 0.004526 | 0.572193 |
| Clostridiales; Ruminococcaceae; | | | | | | | | | |
| Flavonifractor | | | | | | | | | |
| Bacteria; Bacteroidetes; Bacteroidia; | 70 | 0.001608 | 0.078579 | 0.000601 | 0.007335 | 0.042945 | 0.007652 | 0.017223 | 0.310160 |
| Bacteroidales; Porphyromonadaceae; | | | | | | | | | |
| Barnesiella | | | | | | | | | |
| Bacteria; Firmicutes; Tissierellia; | 71 | 0.001557 | 180.170395 | 0.000727 | 0.004641 | 0.214724 | 0.000004 | 0.000026 | 0.026738 |
| Tissierellales; Peptoniphilaceae; | | | | | | | | | |
| Anaerococcus | | | | | | | | | |
| Bacteria; Firmicutes; Bacilli; | 72 | 0.001489 | 21.003684 | 0.001385 | 0.006230 | 0.441718 | 0.000066 | 0.000188 | 0.181818 |
| Bacillales; Not Available; | | | | | | | | | |
| Gemella | | | | | | | | | |
| Bacteria; Proteobacteria; Gamma | 73 | 0.001480 | 1.991330 | 0.002302 | 0.021488 | 0.085890 | 0.001156 | 0.008229 | 0.171123 |
| proteobacteria; Enterobacterales; | | | | | | | | | |
| Enterobacteriaceae; Enterobacter | | | | | | | | | |
| Bacteria; Proteobacteria; Gamma | 74 | 0.001461 | 0.081754 | 0.000024 | 0.000123 | 0.116564 | 0.000290 | 0.001314 | 0.224599 |
| proteobacteria; Pasteurellales; | | | | | | | | | |
| Pasteurellaceae; Other | | | | | | | | | |
| Bacteria; Firmicutes; Clostridia; | 75 | 0.001443 | 17.489209 | 0.000779 | 0.004837 | 0.104294 | 0.000045 | 0.000214 | 0.085561 |
| Clostridiales; Lachnospiraceae; | | | | | | | | | |
| Sellimonas | | | | | | | | | |
| Bacteria; Firmicutes; Erysipelotrichia; | 76 | 0.001408 | 3.234637 | 0.003352 | 0.014227 | 0.306748 | 0.001036 | 0.002439 | 0.588235 |
| Erysipelotrichales; | | | | | | | | | |
| Erysipelotrichaceae; | | | | | | | | | |
| Turicibacter | | | | | | | | | |
| Bacteria; Proteobacteria; Gamma | 77 | 0.001398 | 2.374310 | 0.007749 | 0.038398 | 0.404908 | 0.003264 | 0.019506 | 0.165775 |
| proteobacteria; Enterobacterales; | | | | | | | | | |
| Enterobacteriaceae; Klebsiella | | | | | | | | | |
| Bacteria; Firmicutes; Clostridia; | 78 | 0.001395 | 1.756974 | 0.005762 | 0.015594 | 0.638037 | 0.003280 | 0.007006 | 0.732620 |
| Clostridiales; Peptostreptococcaceae; | | | | | | | | | |
| Other | | | | | | | | | |
| Bacteria; Firmicutes; Tissierellia; | 79 | 0.001352 | 278.158721 | 0.003153 | 0.019435 | 0.196319 | 0.000011 | 0.000061 | 0.048128 |
| Tissierellales; Peptoniphilaceae; | | | | | | | | | |
| Peptoniphilus | | | | | | | | | |
| Bacteria; Proteobacteria; Gamma | 80 | 0.001339 | #DIV/0! | 0.000009 | 0.000026 | 0.159509 | 0.000000 | 0.000000 | 0.000000 |
| proteobacteria; Pasteurellales; | | | | | | | | | |
| Pasteurellaceae; Rodentibacter | | | | | | | | | |
| Bacteria; Firmicutes; Other; Other; | 81 | 0.001270 | 0.284414 | 0.000051 | 0.000146 | 0.306748 | 0.000180 | 0.000400 | 0.395722 |
| Other; Other | | | | | | | | | |
| Bacteria; Firmicutes; Clostridia; | 82 | 0.001229 | 0.016839 | 0.000003 | 0.000024 | 0.030675 | 0.000206 | 0.001044 | 0.208556 |
| Clostridiales; Clostridiaceae | | | | | | | | | |
| 1; Other | | | | | | | | | |
| Bacteria; Firmicutes; Clostridia; | 83 | 0.001194 | 5.274983 | 0.006312 | 0.019947 | 0.484663 | 0.001197 | 0.002673 | 0.604278 |
| Clostridiales; Clostridiaceae | | | | | | | | | |
| 1; Clostridium sensu stricto | | | | | | | | | |
| Bacteria; Firmicutes; Clostridia; | 84 | 0.001174 | 1586439768 | 0.006459 | 0.042897 | 0.233129 | 0.000004 | 0.000039 | 0.016043 |
| Clostridiales; Lachnospiraceae; | | | | | | | | | |
| Faecalimonas | | | | | | | | | |
| Bacteria; Proteobacteria; Gamma | 85 | 0.001171 | 23.242487 | 0.000064 | 0.000244 | 0.226994 | 0.000003 | 0.000037 | 0.005348 |
| proteobacteria; Other; Other; Other | | | | | | | | | |
| Bacteria; Verrucomicrobia; | 86 | 0.001149 | 2.407228 | 0.009958 | 0.079860 | 0.276074 | 0.004137 | 0.015689 | 0.385027 |
| Verrucomicrobiae; | | | | | | | | | |
| Verrucomicrobiales; | | | | | | | | | |
| Akkermansiaceae; Akkermansia | | | | | | | | | |
| Bacteria; Firmicutes; Erysipelotrichia; | 87 | 0.001103 | 3.096942 | 0.003034 | 0.007442 | 0.552147 | 0.000980 | 0.001523 | 0.711230 |
| Erysipelotrichales; | | | | | | | | | |
| Erysipelotrichaceae; | | | | | | | | | |
| Clostridium XVIII | | | | | | | | | |
| Bacteria; Firmicutes; Clostridia; | 88 | 0.001101 | 11.605259 | 0.000059 | 0.000234 | 0.104294 | 0.000005 | 0.000043 | 0.021390 |
| Clostridiales; Clostridiaceae; | | | | | | | | | |
| Lactonifactor | | | | | | | | | |
| Bacteria; Actinobacteria; | 89 | 0.001080 | 1126.206676 | 0.002163 | 0.020292 | 0.251534 | 0.000002 | 0.000016 | 0.016043 |
| Actinobacteria; Micrococcales; | | | | | | | | | |
| Micrococcaceae; Rothia | | | | | | | | | |
| Bacteria; Other; Other; Other; Other; | 90 | 0.001057 | 0.431657 | 0.000114 | 0.000520 | 0.343558 | 0.000263 | 0.000686 | 0.465241 |
| Other | | | | | | | | | |
| Bacteria; Firmicutes; Clostridia; | 91 | 0.001042 | 0.534968 | 0.000068 | 0.000308 | 0.098160 | 0.000127 | 0.000391 | 0.278075 |
| Clostridiales; Catabacteriaceae; | | | | | | | | | |
| Catabacter | | | | | | | | | |
| Bacteria; Firmicutes; Bacilli; | 92 | 0.001035 | 9.743179 | 0.001311 | 0.013830 | 0.073620 | 0.000135 | 0.000962 | 0.064171 |
| Lactobacillales; Leuconostocaceae; | | | | | | | | | |
| Weissella | | | | | | | | | |
| Bacteria; Firmicutes; Clostridia; | 93 | 0.001017 | 11.972136 | 0.003162 | 0.015299 | 0.349693 | 0.000264 | 0.000733 | 0.240642 |
| Clostridiales; Peptostreptococcaceae; | | | | | | | | | |
| Terrisporobacter | | | | | | | | | |
| Bacteria; Bacteroidetes; Bacteroidia; | 94 | 0.001012 | 0.002759 | 0.000000 | 0.000003 | 0.006135 | 0.000076 | 0.000234 | 0.203209 |
| Bacteroidales; Rikenellaceae; | | | | | | | | | |
| Other | | | | | | | | | |
| Bacteria; Firmicutes; Clostridia; | 95 | 0.001005 | 8.367135 | 0.017548 | 0.063530 | 0.490798 | 0.002097 | 0.006178 | 0.534759 |
| Clostridiales; Peptostreptococcaceae; | | | | | | | | | |
| Romboutsia | | | | | | | | | |
| Bacteria; Bacteroidetes; Bacteroidia; | 96 | 0.000991 | 0.359705 | 0.000048 | 0.000225 | 0.085890 | 0.000134 | 0.000555 | 0.155080 |
| Bacteroidales; Porphyromonadaceae; | | | | | | | | | |
| Porphyromonas | | | | | | | | | |
| Bacteria; Firmicutes; Clostridia; | 97 | 0.000961 | #DIV/0! | 0.001633 | 0.009267 | 0.208589 | 0.000000 | 0.000000 | 0.000000 |
| Clostridiales; Peptostreptococcaceae; | | | | | | | | | |
| Clostridium XI | | | | | | | | | |
| Bacteria; Actinobacteria; | 98 | 0.000960 | 43.088143 | 0.000851 | 0.004649 | 0.226994 | 0.000020 | 0.000079 | 0.080214 |
| Actinobacteria; Coriobacteriales; | | | | | | | | | |
| Coriobacteriaceae; Atopobium | | | | | | | | | |
| Bacteria; Actinobacteria; | 99 | 0.000944 | 75.195507 | 0.000616 | 0.002338 | 0.251534 | 0.000008 | 0.000036 | 0.058824 |
| Coriobacteriia; Coriobacteriales; | | | | | | | | | |
| Atopobiaceae; Atopobium | | | | | | | | | |
| Bacteria; Firmicutes; Clostridia; | 100 | 0.000915 | #DIV/0! | 0.000072 | 0.000538 | 0.092025 | 0.000000 | 0.000000 | 0.000000 |
| Clostridiales; Ruminococcaceae; | | | | | | | | | |
| Anaeromassilibacillus | | | | | | | | | |

CDI = Clostridiodes difficile Infection;
Ctrl = Control;
P- = Pediatric;
RFFR = Random Forest Feature Rank;
# = Rank;
MDA = Mean Decrease Accuracy;
FC = Fold Change;
M = Mean;
SD = Standard Deviation;
DP = Detection Prevalence;

TABLE 17
Pediatric CDI vs Adult CDI delineation features - (taxonomy)

| Feature | | | FC | | | | | | |
|---|---|---|---|---|---|---|---|---|---|
| (Taxonomic order: Kingdom; | | | CDI | Abundance | | | Abundance | | |
| Phylum; Class; Order; | RFFR | | vs | (P-CDI) | | | (A-CDI) | | |
| Family; Genus) | # | MDA | CDI | M | SD | DP | M | SD | DP |
| Bacteria; Proteobacteria; Gamma | 1 | 0.037538 | 151.080905 | 0.000000 | 0.000002 | 0.000954 | 0.000009 | 0.000026 | 0.159509 |
| proteobacteria; Pasteurellales; | | | | | | | | | |
| Pasteurellaceae; Rodentibacter | | | | | | | | | |
| Bacteria; Proteobacteria; | 2 | 0.025568 | 0.712858 | 0.000016 | 0.000387 | 0.006679 | 0.000012 | 0.000050 | 0.159509 |
| Betaproteobacteria; Burkholderiales; | | | | | | | | | |
| Sutterellaceae; Turicimonas | | | | | | | | | |
| Bacteria; Actinobacteria; | 3 | 0.018837 | 11.206714 | 0.000572 | 0.004184 | 0.394084 | 0.006408 | 0.030049 | 0.613497 |
| Actinobacteria; Actinomycetales; | | | | | | | | | |
| Actinomycetaceae; Schaalia | | | | | | | | | |
| Bacteria; Firmicutes; Clostridia; | 4 | 0.017826 | 11.661045 | 0.001505 | 0.008536 | 0.330153 | 0.017548 | 0.063530 | 0.490798 |
| Clostridiales; Peptostreptococcaceae; | | | | | | | | | |
| Romboutsia | | | | | | | | | |
| Bacteria; Bacteroidetes; Bacteroidia; | 5 | 0.017381 | 0.537135 | 0.175453 | 0.210442 | 0.922710 | 0.094242 | 0.201046 | 0.779141 |
| Bacteroidales; Bacteroidaceae; | | | | | | | | | |
| Bacteroides | | | | | | | | | |
| Bacteria; Firmicutes; Clostridia; | 6 | 0.017077 | 1.794149 | 0.050846 | 0.086265 | 0.885496 | 0.091226 | 0.150407 | 0.889571 |
| Clostridiales; Lachnospiraceae; | | | | | | | | | |
| Blautia | | | | | | | | | |
| Bacteria; Firmicutes; Bacilli; | 7 | 0.016274 | 37.669569 | 0.000124 | 0.001927 | 0.083969 | 0.004680 | 0.032716 | 0.233129 |
| Lactobacillales; Aerococcaceae; | | | | | | | | | |
| Abiotrophia | | | | | | | | | |
| Bacteria; Firmicutes; | 8 | 0.015806 | 2.407213 | 0.013303 | 0.033415 | 0.801527 | 0.032024 | 0.068740 | 0.809816 |
| Erysipelotrichia; Erysipelotrichales; | | | | | | | | | |
| Erysipelotrichaceae; | | | | | | | | | |
| Erysipelatoclostridium | | | | | | | | | |
| Bacteria; Firmicutes; Clostridia; | 9 | 0.015389 | 4.414909 | 0.004632 | 0.014406 | 0.599237 | 0.020449 | 0.063547 | 0.619632 |
| Clostridiales; Lachnospiraceae; | | | | | | | | | |
| Anaerostipes | | | | | | | | | |
| Bacteria; Firmicutes; Clostridia; | 10 | 0.014791 | 0.531764 | 0.010273 | 0.025945 | 0.808206 | 0.005463 | 0.025266 | 0.607362 |
| Clostridiales; Lachnospiraceae; | | | | | | | | | |
| Clostridium XIVa | | | | | | | | | |
| Bacteria; Actinobacteria; | 11 | 0.014684 | 3.174081 | 0.001956 | 0.005797 | 0.514313 | 0.006208 | 0.014624 | 0.582822 |
| Coriobacteriia; Eggerthellales; | | | | | | | | | |
| Eggerthellaceae; Eggerthella | | | | | | | | | |
| Bacteria; Firmicutes; Bacilli; | 12 | 0.012805 | 2.205140 | 0.033469 | 0.082817 | 0.907443 | 0.073805 | 0.133505 | 0.944785 |
| Lactobacillales; Streptococcaceae; | | | | | | | | | |
| Streptococcus | | | | | | | | | |
| Bacteria; Firmicutes; Bacilli; | 13 | 0.012788 | 103.234362 | 0.000008 | 0.000222 | 0.006679 | 0.000865 | 0.006796 | 0.110429 |
| Lactobacillales; Aerococcaceae; | | | | | | | | | |
| Other | | | | | | | | | |
| Bacteria; Bacteroidetes; Bacteroidia; | 14 | 0.011740 | 0.223093 | 0.015206 | 0.045358 | 0.614504 | 0.003392 | 0.013174 | 0.306748 |
| Bacteroidales; Porphyromonadaceae; | | | | | | | | | |
| Parabacteroides | | | | | | | | | |
| Bacteria; Firmicutes; Clostridia; | 15 | 0.011507 | 1.561592 | 0.008139 | 0.021296 | 0.667939 | 0.012709 | 0.032446 | 0.509202 |
| Clostridiales; Lachnospiraceae; | | | | | | | | | |
| Lachnospiracea_{—}incertae_{—}sedis | | | | | | | | | |
| Bacteria; Proteobacteria; Gamma | 16 | 0.011411 | 0.634772 | 0.071029 | 0.140941 | 0.880725 | 0.045087 | 0.151893 | 0.840491 |
| proteobacteria; Enterobacteriales; | | | | | | | | | |
| Enterobacteriaceae; Other | | | | | | | | | |
| Bacteria; Actinobacteria; | 17 | 0.011042 | 4.850911 | 0.000980 | 0.003992 | 0.566794 | 0.004754 | 0.018003 | 0.644172 |
| Actinobacteria; Actinomycetales; | | | | | | | | | |
| Actinomycetaceae; Actinomyces | | | | | | | | | |
| Bacteria; Firmicutes; Clostridia; | 18 | 0.010988 | 2.639563 | 0.004688 | 0.019981 | 0.477099 | 0.012375 | 0.031853 | 0.625767 |
| Clostridiales; Peptostreptococcaceae; | | | | | | | | | |
| Intestinibacter | | | | | | | | | |
| Bacteria; Proteobacteria; Gamma | 19 | 0.010954 | 0.330765 | 0.023428 | 0.075459 | 0.644084 | 0.007749 | 0.038398 | 0.404908 |
| proteobacteria; Enterobacterales; | | | | | | | | | |
| Enterobacteriaceae; Klebsiella | | | | | | | | | |
| Bacteria; Candidatus | 20 | 0.010741 | 8.205493 | 0.000073 | 0.001179 | 0.065840 | 0.000595 | 0.004970 | 0.184049 |
| Saccharibacteria; | | | | | | | | | |
| Saccharibacteria_genera_incertae_sedis; | | | | | | | | | |
| Other; Other; Other | | | | | | | | | |
| Bacteria; Firmicutes; Erysipelotrichia; | 21 | 0.010622 | #DIV/0! | 0.000000 | 0.000000 | 0.000000 | 0.000002 | 0.000009 | 0.061350 |
| Erysipelotrichales; Erysipelotrichaceae; | | | | | | | | | |
| Faecalibaculum | | | | | | | | | |
| Bacteria; Proteobacteria; Gamma | 22 | 0.010398 | 1.613390 | 0.030797 | 0.091460 | 0.675573 | 0.049688 | 0.127352 | 0.730061 |
| proteobacteria; Enterobacterales; | | | | | | | | | |
| Enterobacteriaceae; Escherichia | | | | | | | | | |
| Bacteria; Firmicutes; Bacilli; Bacillales; | 23 | 0.010168 | 3.224048 | 0.000430 | 0.003268 | 0.271947 | 0.001385 | 0.006230 | 0.441718 |
| Not Available; Gemella | | | | | | | | | |
| Bacteria; Firmicutes; Clostridia; | 24 | 0.010037 | 0.437115 | 0.003736 | 0.020683 | 0.507634 | 0.001633 | 0.009267 | 0.208589 |
| Clostridiales; Peptostreptococcaceae; | | | | | | | | | |
| Clostridium XI | | | | | | | | | |
| Bacteria; Deferribacteres; | 25 | 0.009997 | 17.294619 | 0.000000 | 0.000005 | 0.001908 | 0.000003 | 0.000013 | 0.073620 |
| Deferribacteres; Deferribacterales; | | | | | | | | | |
| Deferribacteraceae; Mucispirillum | | | | | | | | | |
| Bacteria; Firmicutes; Bacilli; | 26 | 0.009757 | 0.880286 | 0.041795 | 0.118159 | 0.806298 | 0.036792 | 0.134244 | 0.754601 |
| Lactobacillales; Lactobacilloae; | | | | | | | | | |
| Lactobacillus | | | | | | | | | |
| Bacteria; Firmicutes; Bacilli; | 27 | 0.009695 | 3.193299 | 0.000521 | 0.003417 | 0.382634 | 0.001663 | 0.005044 | 0.552147 |
| Lactobacillales; Carnobacteriaceae; | | | | | | | | | |
| Granulicatella | | | | | | | | | |
| Bacteria; Firmicutes; Erysipelotrichia; | 28 | 0.009667 | 11.439285 | 0.000635 | 0.004394 | 0.109733 | 0.007263 | 0.039105 | 0.214724 |
| Erysipelotrichales; | | | | | | | | | |
| Erysipelotrichaceae; Longicatena | | | | | | | | | |
| Bacteria; Proteobacteria; Epsilon | 29 | 0.008849 | 0.087054 | 0.000041 | 0.000796 | 0.014313 | 0.000004 | 0.000012 | 0.092025 |
| proteobacteria; Campylobacterales; | | | | | | | | | |
| Helicobacteraceae; Helicobacter | | | | | | | | | |
| Bacteria; Firmicutes; Bacilli; | 30 | 0.008768 | 1.270599 | 0.087313 | 0.178981 | 0.854962 | 0.110940 | 0.208194 | 0.791411 |
| Lactobacillales; Enterococcaceae; | | | | | | | | | |
| Enterococcus | | | | | | | | | |
| Bacteria; Firmicutes; Clostridia; | 31 | 0.008158 | 0.219799 | 0.006744 | 0.026190 | 0.504771 | 0.001482 | 0.007581 | 0.208589 |
| Clostridiales; Lachnospiraceae; | | | | | | | | | |
| Ruminococcus2 | | | | | | | | | |
| Bacteria; Firmicutes; Clostridia; | 32 | 0.008147 | 0.685279 | 0.017946 | 0.035471 | 0.865458 | 0.012298 | 0.026170 | 0.791411 |
| Clostridiales; Lachnospiraceae; | | | | | | | | | |
| Other | | | | | | | | | |
| Bacteria; Actinobacteria; Actinobacteria; | 33 | 0.007975 | 5.379831 | 0.000175 | 0.001109 | 0.203244 | 0.000941 | 0.006184 | 0.355828 |
| Corynebacteriales; Corynebacteriaceae; | | | | | | | | | |
| Corynebacterium | | | | | | | | | |
| Bacteria; Firmicutes; Bacilli; | 34 | 0.007947 | 0.614930 | 0.011599 | 0.049684 | 0.661260 | 0.007133 | 0.018683 | 0.760736 |
| Lactobacillales; Other; Other | | | | | | | | | |
| Bacteria; Firmicutes; Negativicutes; | 35 | 0.007882 | 0.969644 | 0.015102 | 0.052413 | 0.614504 | 0.014643 | 0.045727 | 0.705521 |
| Veillonellales; Veillonellaceae; | | | | | | | | | |
| Veillonella | | | | | | | | | |
| Bacteria; Firmicutes; Clostridia; | 36 | 0.007833 | 1.293561 | 0.003973 | 0.011225 | 0.730916 | 0.005139 | 0.016770 | 0.693252 |
| Clostridiales; Other; Other | | | | | | | | | |
| Bacteria; Firmicutes; Negativicutes; | 37 | 0.007592 | 0.453938 | 0.010436 | 0.042733 | 0.619275 | 0.004737 | 0.037434 | 0.453988 |
| Selenomonadales; Veillonellaceae; | | | | | | | | | |
| Veillonella | | | | | | | | | |
| Bacteria; Actinobacteria; | 38 | 0.007582 | 12.594796 | 0.000068 | 0.000474 | 0.122137 | 0.000851 | 0.004649 | 0.226994 |
| Actinobacteria; Coriobacteriales; | | | | | | | | | |
| Coriobacteriaceae; Atopobium | | | | | | | | | |
| Bacteria; Firmicutes; Clostridia; | 39 | 0.007539 | 0.653632 | 0.011966 | 0.039074 | 0.724237 | 0.007821 | 0.031595 | 0.693252 |
| Clostridiales; Peptostreptococcaceae; | | | | | | | | | |
| Clostridioides | | | | | | | | | |
| Bacteria; Other; Other; Other; Other; | 40 | 0.007354 | 0.203238 | 0.000559 | 0.002552 | 0.521947 | 0.000114 | 0.000520 | 0.343558 |
| Other | | | | | | | | | |
| Bacteria; Firmicutes; Clostridia; | 41 | 0.007304 | 1.041457 | 0.005533 | 0.023519 | 0.677481 | 0.005762 | 0.015594 | 0.638037 |
| Clostridiales; Peptostreptococcaceae; | | | | | | | | | |
| Other | | | | | | | | | |
| Bacteria; Verrucomicrobia; | 42 | 0.007236 | 0.648843 | 0.018257 | 0.071411 | 0.470420 | 0.011846 | 0.083730 | 0.226994 |
| Verrucomicrobiae; Verrucomicrobiales; | | | | | | | | | |
| Verrucomicrobiaceae; Akkermansia | | | | | | | | | |
| Bacteria; Firmicutes; Bacilli; Other; | 43 | 0.007148 | 0.735678 | 0.000541 | 0.004864 | 0.383588 | 0.000398 | 0.000891 | 0.503067 |
| Other; Other | | | | | | | | | |
| Bacteria; Firmicutes; Clostridia; | 44 | 0.007078 | 8.528665 | 0.000048 | 0.000974 | 0.015267 | 0.000411 | 0.002207 | 0.061350 |
| Clostridiales; Ruminococcaceae; | | | | | | | | | |
| Agathobaculum | | | | | | | | | |
| Bacteria; Firmicutes; Clostridia; | 45 | 0.007066 | 1.137991 | 0.013426 | 0.032788 | 0.810115 | 0.015279 | 0.045057 | 0.711656 |
| Clostridiales; Lachnospiraceae; | | | | | | | | | |
| Lachnoclostridium | | | | | | | | | |
| Bacteria; Actinobacteria; Actinobacteria; | 46 | 0.006869 | 1.952126 | 0.012501 | 0.043977 | 0.559160 | 0.024403 | 0.085315 | 0.490798 |
| Bifidobacteriales; Bifidobacteriaceae; | | | | | | | | | |
| Bifidobacterium | | | | | | | | | |
| Bacteria; Firmicutes; Clostridia; | 47 | 0.006849 | 2.847151 | 0.001215 | 0.006227 | 0.168893 | 0.003460 | 0.017320 | 0.263804 |
| Clostridiales; Lachnospiraceae; | | | | | | | | | |
| Tyzzerella | | | | | | | | | |
| Bacteria; Firmicutes; Bacilli; | 48 | 0.006523 | 0.713944 | 0.006149 | 0.029300 | 0.490458 | 0.004390 | 0.026302 | 0.509202 |
| Lactobacillales; Enterococcaceae; | | | | | | | | | |
| Other | | | | | | | | | |
| Bacteria; Proteobacteria; Gamma | 49 | 0.006314 | 1.003815 | 0.012077 | 0.063124 | 0.492366 | 0.012123 | 0.075931 | 0.368098 |
| proteobacteria; Enterobacterales; | | | | | | | | | |
| Enterobacteriaceae; Citrobacter | | | | | | | | | |
| Bacteria; Firmicutes; Clostridia; | 50 | 0.006263 | 0.897610 | 0.013901 | 0.042917 | 0.643130 | 0.012477 | 0.035348 | 0.638037 |
| Clostridiales; Clostridiaceae; | | | | | | | | | |
| Clostridium | | | | | | | | | |
| Bacteria; Bacteroidetes; Bacteroidia; | 51 | 0.006233 | 0.595628 | 0.017656 | 0.064847 | 0.557252 | 0.010516 | 0.078929 | 0.300613 |
| Bacteroidales; Rikenellaceae; | | | | | | | | | |
| Alistipes | | | | | | | | | |
| Bacteria; Firmicutes; Clostridia; | 52 | 0.006158 | 2.575898 | 0.001978 | 0.009577 | 0.395038 | 0.005096 | 0.026154 | 0.417178 |
| Clostridiales; Clostridiaceae; | | | | | | | | | |
| Hungatella | | | | | | | | | |
| Bacteria; Firmicutes; Clostridia; | 53 | 0.005948 | 2.072889 | 0.001971 | 0.010690 | 0.288168 | 0.004086 | 0.015414 | 0.202454 |
| Clostridiales; Lachnospiraceae; | | | | | | | | | |
| Fusicatenibacter | | | | | | | | | |
| Bacteria; Firmicutes; Clostridia; | 54 | 0.005875 | 2.656411 | 0.001190 | 0.006108 | 0.289122 | 0.003162 | 0.015299 | 0.349693 |
| Clostridiales; Peptostreptococcaceae; | | | | | | | | | |
| Terrisporobacter | | | | | | | | | |
| Bacteria; Firmicutes; Erysipelotrichia; | 55 | 0.005854 | 0.432155 | 0.007020 | 0.025301 | 0.643130 | 0.003034 | 0.007442 | 0.552147 |
| Erysipelotrichales; Erysipelotrichaceae; | | | | | | | | | |
| Clostridium XVIII | | | | | | | | | |
| Bacteria; Verrucomicrobia; | 56 | 0.005717 | 0.767486 | 0.012974 | 0.062238 | 0.416985 | 0.009958 | 0.079860 | 0.276074 |
| Verrucomicrobiae; Verrucomicrobiales; | | | | | | | | | |
| Akkermansiaceae; Akkermansia | | | | | | | | | |
| Bacteria; Firmicutes; Clostridia; | 57 | 0.005618 | 0.898806 | 0.007022 | 0.030846 | 0.540076 | 0.006312 | 0.019947 | 0.484663 |
| Clostridiales; Clostridiaceae | | | | | | | | | |
| 1; Clostridium sensu stricto | | | | | | | | | |
| Bacteria; Firmicutes; Bacilli; | 58 | 0.005548 | 1.816253 | 0.001158 | 0.008212 | 0.312977 | 0.002103 | 0.008670 | 0.355828 |
| Lactobacillales; Streptococcaceae; | | | | | | | | | |
| Lactococcus | | | | | | | | | |
| Bacteria; Firmicutes; Clostridia; | 59 | 0.005546 | 2.107635 | 0.002959 | 0.017495 | 0.396947 | 0.006237 | 0.029087 | 0.312883 |
| Clostridiales; Lachnospiraceae; | | | | | | | | | |
| Eisenbergiella | | | | | | | | | |
| Bacteria; Firmicutes; Clostridia; | 60 | 0.005541 | 5.110983 | 0.001534 | 0.006380 | 0.385496 | 0.007839 | 0.041596 | 0.349693 |
| Clostridiales; Eubacteriaceae; | | | | | | | | | |
| Eubacterium | | | | | | | | | |
| Bacteria; Bacteroidetes; Bacteroidia; | 61 | 0.005518 | 0.537346 | 0.013330 | 0.041049 | 0.558206 | 0.007163 | 0.038013 | 0.374233 |
| Bacteroidales; Tannerellaceae; | | | | | | | | | |
| Parabacteroides | | | | | | | | | |
| Bacteria; Actinobacteria; Coriobacteriia; | 62 | 0.005414 | 3.588974 | 0.000172 | 0.001659 | 0.198473 | 0.000616 | 0.002338 | 0.251534 |
| Coriobacteriales; Atopobiaceae; | | | | | | | | | |
| Atopobium | | | | | | | | | |
| Bacteria; Firmicutes; Clostridia; | 63 | 0.005301 | 0.810345 | 0.005449 | 0.026062 | 0.585878 | 0.004416 | 0.017277 | 0.423313 |
| Clostridiales; Ruminococcaceae; | | | | | | | | | |
| Ruthenibacterium | | | | | | | | | |
| Bacteria; Proteobacteria; Gamma | 64 | 0.005283 | 26.586432 | 0.000201 | 0.004162 | 0.010496 | 0.005340 | 0.068044 | 0.073620 |
| proteobacteria; Enterobacterales; | | | | | | | | | |
| Yersiniaceae; Yersinia | | | | | | | | | |
| Bacteria; Firmicutes; Clostridia; | 65 | 0.005199 | 20.835218 | 0.000057 | 0.000645 | 0.067748 | 0.001183 | 0.011757 | 0.098160 |
| Clostridiales; Clostridiales_Incertae | | | | | | | | | |
| Sedis XI; Finegoldia | | | | | | | | | |
| Bacteria; Firmicutes; Clostridia; | 66 | 0.005010 | 2.292412 | 0.002818 | 0.014333 | 0.268130 | 0.006459 | 0.042897 | 0.233129 |
| Clostridiales; Lachnospiraceae; | | | | | | | | | |
| Faecalimonas | | | | | | | | | |
| Bacteria; Actinobacteria; | 67 | 0.004778 | 8.632569 | 0.000003 | 0.000050 | 0.023855 | 0.000025 | 0.000116 | 0.092025 |
| Actinobacteria; Propionibacteriales; | | | | | | | | | |
| Propionibacteriaceae; | | | | | | | | | |
| Pseudopropionibacterium | | | | | | | | | |
| Bacteria; Actinobacteria; Actinobacteria; | 68 | 0.004733 | 9.367740 | 0.000231 | 0.001642 | 0.341603 | 0.002163 | 0.020292 | 0.251534 |
| Micrococcales; Micrococcaceae; | | | | | | | | | |
| Rothia | | | | | | | | | |
| Bacteria; Firmicutes; Clostridia; | 69 | 0.004676 | 0.673454 | 0.003217 | 0.009467 | 0.612595 | 0.002166 | 0.008636 | 0.484663 |
| Clostridiales; Ruminococcaceae; | | | | | | | | | |
| Flavonifractor | | | | | | | | | |
| Bacteria; Firmicutes; Clostridia; | 70 | 0.004577 | 1.717864 | 0.002840 | 0.012164 | 0.349237 | 0.004879 | 0.018782 | 0.269939 |
| Clostridiales; Ruminococcaceae; | | | | | | | | | |
| Ruminococcus | | | | | | | | | |
| Bacteria; Firmicutes; Clostridia; | 71 | 0.004567 | 2.859750 | 0.000482 | 0.005946 | 0.104962 | 0.001378 | 0.007208 | 0.134969 |
| Clostridiales; Lachnospiraceae; | | | | | | | | | |
| Robinsoniella | | | | | | | | | |
| Bacteria; Firmicutes; Clostridia; | 72 | 0.004434 | 0.840682 | 0.011117 | 0.042802 | 0.461832 | 0.009346 | 0.037604 | 0.337423 |
| Clostridiales; Ruminococcaceae; | | | | | | | | | |
| Faecalibacterium | | | | | | | | | |
| Bacteria; Firmicutes; Erysipelotrichia; | 73 | 0.004410 | 2.179446 | 0.001538 | 0.012407 | 0.292939 | 0.003352 | 0.014227 | 0.306748 |
| Erysipelotrichales; Erysipelotrichaceae; | | | | | | | | | |
| Turicibacter | | | | | | | | | |
| Bacteria; Proteobacteria; Gamma | 74 | 0.004235 | 0.527508 | 0.023907 | 0.124063 | 0.266221 | 0.012611 | 0.084066 | 0.141104 |
| proteobacteria; Pseudomonadales; | | | | | | | | | |
| Pseudomonadaceae; Pseudomonas | | | | | | | | | |
| Bacteria; Firmicutes; Clostridia; | 75 | 0.004167 | 0.750109 | 0.010395 | 0.037868 | 0.557252 | 0.007797 | 0.024579 | 0.472393 |
| Clostridiales; Ruminococcaceae; | | | | | | | | | |
| Other | | | | | | | | | |
| Bacteria; Firmicutes; Tissierellia; | 76 | 0.004150 | 5.907737 | 0.000306 | 0.002713 | 0.166031 | 0.001809 | 0.016334 | 0.226994 |
| Tissierellales; Peptoniphilaceae; | | | | | | | | | |
| Finegoldia | | | | | | | | | |
| Bacteria; Actinobacteria; Actinobacteria; | 77 | 0.004137 | 132.236072 | 0.000001 | 0.000007 | 0.009542 | 0.000073 | 0.000758 | 0.055215 |
| Actinomycetales; Propionibacteriaceae; | | | | | | | | | |
| Other | | | | | | | | | |
| Bacteria; Firmicutes; Negativicutes; | 78 | 0.004094 | 1.456619 | 0.003390 | 0.013104 | 0.270038 | 0.004939 | 0.021585 | 0.165644 |
| Acidaminococcales; | | | | | | | | | |
| Acidaminococcaceae; | | | | | | | | | |
| Phascolarctobacterium | | | | | | | | | |
| Bacteria; Proteobacteria; Gamma | 79 | 0.004070 | #DIV/0! | 0.000000 | 0.000000 | 0.000000 | 0.000055 | 0.000409 | 0.042945 |
| proteobacteria; Enterobacterales; | | | | | | | | | |
| Serratia; Serratia liquefaciens | | | | | | | | | |
| Bacteria; Bacteroidetes; Bacteroidia; | 80 | 0.004031 | 0.274976 | 0.008745 | 0.050290 | 0.405534 | 0.002405 | 0.013350 | 0.226994 |
| Bacteroidales; Prevotellaceae; | | | | | | | | | |
| Prevotella | | | | | | | | | |
| Bacteria; Firmicutes; Clostridia; | 81 | 0.003857 | 1.338720 | 0.000051 | 0.000746 | 0.068702 | 0.000068 | 0.000308 | 0.098160 |
| Clostridiales; Catabacteriaceae; | | | | | | | | | |
| Catabacter | | | | | | | | | |
| Bacteria; Firmicutes; Other; Other; | 82 | 0.003677 | 0.128617 | 0.000398 | 0.002477 | 0.411260 | 0.000051 | 0.000146 | 0.306748 |
| Other; Other | | | | | | | | | |
| Bacteria; Firmicutes; Clostridia; | 83 | 0.003648 | 4.203475 | 0.001122 | 0.005263 | 0.240458 | 0.004717 | 0.031952 | 0.177914 |
| Clostridiales; Lachnospiraceae; | | | | | | | | | |
| Dorea | | | | | | | | | |
| Bacteria; Proteobacteria; Betaproteobacteria; | 84 | 0.003517 | 2.284352 | 0.000242 | 0.003895 | 0.072519 | 0.000552 | 0.004157 | 0.128834 |
| Neisseriales; Neisseriaceae; | | | | | | | | | |
| Eikenella | | | | | | | | | |
| Bacteria; Firmicutes; Negativicutes; | 85 | 0.003359 | 1.900190 | 0.002501 | 0.010178 | 0.306298 | 0.004752 | 0.031692 | 0.220859 |
| Veillonellales; Veillonellaceae; | | | | | | | | | |
| Dialister | | | | | | | | | |
| Bacteria; Proteobacteria; Gamma | 86 | 0.003323 | 0.310934 | 0.002614 | 0.012587 | 0.342557 | 0.000813 | 0.003609 | 0.319018 |
| proteobacteria; Pasteurellales; | | | | | | | | | |
| Pasteurellaceae; Haemophilus | | | | | | | | | |
| Bacteria; Firmicutes; Erysipelotrichia; | 87 | 0.003322 | 0.744685 | 0.000660 | 0.004626 | 0.208015 | 0.000492 | 0.002420 | 0.214724 |
| Erysipelotrichales; Erysipelotrichaceae; | | | | | | | | | |
| Coprobacillus | | | | | | | | | |
| Bacteria; Firmicutes; Tissierellia; | 88 | 0.003308 | 2.879399 | 0.001095 | 0.008235 | 0.161260 | 0.003153 | 0.019435 | 0.196319 |
| Tissierellales; Peptoniphilaceae; | | | | | | | | | |
| Peptoniphilus | | | | | | | | | |
| Bacteria; Firmicutes; Clostridia; | 89 | 0.003308 | 1.645935 | 0.000952 | 0.008358 | 0.133588 | 0.001567 | 0.010485 | 0.239264 |
| Clostridiales; Peptostreptococcaceae; | | | | | | | | | |
| Peptostreptococcus | | | | | | | | | |
| Bacteria; Proteobacteria; Gamma | 90 | 0.003249 | 0.175053 | 0.000364 | 0.001037 | 0.373092 | 0.000064 | 0.000244 | 0.226994 |
| proteobacteria; Other; Other; | | | | | | | | | |
| Other | | | | | | | | | |
| Bacteria; Firmicutes; Bacilli; | 91 | 0.003152 | 0.134195 | 0.003189 | 0.038590 | 0.308206 | 0.000428 | 0.003028 | 0.251534 |
| Bacillales; Staphylococcaceae; | | | | | | | | | |
| Staphylococcus | | | | | | | | | |
| Bacteria; Firmicutes; Bacilli; | 92 | 0.003143 | 24.565543 | 0.000030 | 0.000212 | 0.083969 | 0.000736 | 0.005383 | 0.104294 |
| Lactobacillales; Leuconostocaceae; | | | | | | | | | |
| Leuconostoc | | | | | | | | | |
| Bacteria; Fusobacteria; Fusobacteriia; | 93 | 0.003109 | 0.259507 | 0.009036 | 0.042807 | 0.392176 | 0.002345 | 0.012720 | 0.269939 |
| Fusobacteriales; Fusobacteriaceae; | | | | | | | | | |
| Fusobacterium | | | | | | | | | |
| Bacteria; Firmicutes; Clostridia; | 94 | 0.003107 | 2.368769 | 0.000044 | 0.000532 | 0.064885 | 0.000104 | 0.000590 | 0.079755 |
| Clostridiales; Christensenellaceae; | | | | | | | | | |
| Christensenella | | | | | | | | | |
| Bacteria; Firmicutes; Negativicutes; | 95 | 0.003105 | 1.786312 | 0.000785 | 0.006558 | 0.164122 | 0.001402 | 0.011199 | 0.190184 |
| Veillonellales; Veillonellaceae; | | | | | | | | | |
| Megasphaera | | | | | | | | | |
| Bacteria; Proteobacteria; Gamma | 96 | 0.002968 | 0.002014 | 0.004347 | 0.038064 | 0.219466 | 0.000009 | 0.000056 | 0.061350 |
| proteobacteria; Enterobacterales; | | | | | | | | | |
| Enterobacteriaceae; Salmonella | | | | | | | | | |
| Bacteria; Firmicutes; Bacilli; | 97 | 0.002926 | 0.817985 | 0.000416 | 0.003045 | 0.183206 | 0.000340 | 0.003049 | 0.134969 |
| Lactobacillales; Lactobacilloae; | | | | | | | | | |
| Other | | | | | | | | | |
| Bacteria; Actinobacteria; Actinobacteria; | 98 | 0.002819 | 0.190002 | 0.000943 | 0.003455 | 0.330153 | 0.000179 | 0.000538 | 0.282209 |
| Coriobacteriales; Coriobacteriaceae; | | | | | | | | | |
| Eggerthella | | | | | | | | | |
| Bacteria; Firmicutes; Clostridia; | 99 | 0.002815 | 1.077056 | 0.001097 | 0.006183 | 0.292939 | 0.001182 | 0.006649 | 0.177914 |
| Clostridiales; Lachnospiraceae; | | | | | | | | | |
| [Eubacterium] rectale group | | | | | | | | | |
| Bacteria; Firmicutes; Clostridia; | 100 | 0.002815 | 0.360840 | 0.003179 | 0.012804 | 0.391221 | 0.001147 | 0.006662 | 0.245399 |
| Clostridiales; Lachnospiraceae; | | | | | | | | | |
| Roseburia | | | | | | | | | |

CDI = Clostridiodes difficile Infection;
Ctrl = Control;
P- = Pediatric;
A- = Adult;
RFFR = Random Forest Feature Rank;
# = Rank;
MDA = Mean Decrease Accuracy;
FC = Fold Change;
M = Mean;
SD = Standard Deviation;
DP = Detection Prevalence;

TABLE 18
MetaCyc (2022) defined metabolic pathways identified as features

| Pathway ID | Pathway description |
|---|---|
| 1CMET2-PWY | N10-formyl-tetrahydrofolate biosynthesis |
| 3-HYDROXYPHENYLACETATE- | 4-hydroxyphenylacetate degradation |
| DEGRADATION-PWY | |
| AEROBACTINSYN-PWY | aerobactin biosynthesis |
| ALL-CHORISMATE-PWY | superpathway of chorismate metabolism |
| ANAEROFRUCAT-PWY | homolactic fermentation |
| ANAGLYCOLYSIS-PWY | glycolysis III (from glucose) |
| ARGDEG-PWY | superpathway of L-arginine, putrescine, and 4- |
| | aminobutanoate degradation |
| ARGORNPROST-PWY | arginine, ornithine and proline interconversion |
| ARGSYNBSUB-PWY | L-arginine biosynthesis II (acetyl cycle) |
| ARGSYN-PWY | L-arginine biosynthesis I (via L-ornithine) |
| ARO-PWY | chorismate biosynthesis I |
| ASPASN-PWY | superpathway of L-aspartate and L-asparagine |
| | biosynthesis |
| AST-PWY | L-arginine degradation II (AST pathway) |
| BRANCHED-CHAIN-AA-SYN- | superpathway of branched amino acid biosynthesis |
| PWY | |
| CALVIN-PWY | Calvin-Benson-Bassham cycle |
| CENTFERM-PWY | pyruvate fermentation to butanoate |
| CHLOROPHYLL-SYN | chlorophyllide a biosynthesis I (aerobic, light-dependent) |
| COA-PWY | coenzyme A biosynthesis I |
| COBALSYN-PWY | adenosylcobalamin salvage from cobinamide I |
| CODH-PWY | reductive acetyl coenzyme A pathway |
| COMPLETE-ARO-PWY | superpathway of aromatic amino acid biosynthesis |
| DAPLYSINESYN-PWY | L-lysine biosynthesis I |
| DENOVOPURINE2-PWY | superpathway of purine nucleotides de novo biosynthesis II |
| DHGLUCONATE-PYR-CAT- | glucose degradation (oxidative) |
| PWY | |
| DTDPRHAMSYN-PWY | dTDP-L-rhamnose biosynthesis I |
| ECASYN-PWY | enterobacterial common antigen biosynthesis |
| ENTBACSYN-PWY | enterobactin biosynthesis |
| FAO-PWY | fatty acid &beta;-oxidation I |
| FASYN-ELONG-PWY | fatty acid elongation -- saturated |
| FASYN-INITIAL-PWY | superpathway of fatty acid biosynthesis initiation |
| | (E. coli) |
| FERMENTATION-PWY | mixed acid fermentation |
| FOLSYN-PWY | superpathway of tetrahydrofolate biosynthesis and |
| | salvage |
| FUCCAT-PWY | fucose degradation |
| FUC-RHAMCAT-PWY | superpathway of fucose and rhamnose degradation |
| GALACTARDEG-PWY | D-galactarate degradation I |
| GALACT-GLUCUROCAT-PWY | superpathway of hexuronide and hexuronate degradation |
| GALACTUROCAT-PWY | D-galacturonate degradation I |
| GLCMANNANAUT-PWY | superpathway of N-acetylglucosamine, N- |
| | acetylmannosamine and N-acetylneuraminate |
| | degradation |
| GLUCARDEG-PWY | D-glucarate degradation I |
| GLUCARGALACTSUPER- | superpathway of D-glucarate and D-galactarate |
| PWY | degradation |
| GLUCONEO-PWY | gluconeogenesis I |
| GLUCOSE1PMETAB-PWY | glucose and glucose-1-phosphate degradation |
| GLUCUROCAT-PWY | superpathway of &beta;-D-glucuronide and D- |
| | glucuronate degradation |
| GLUTORN-PWY | L-ornithine biosynthesis |
| GLYCOCAT-PWY | glycogen degradation I (bacterial) |
| GLYCOGENSYNTH-PWY | glycogen biosynthesis I (from ADP-D-Glucose) |
| GLYCOL-GLYOXDEG-PWY | superpathway of glycol metabolism and degradation |
| GLYCOLYSIS | glycolysis I (from glucose 6-phosphate) |
| GLYCOLYSIS-E-D | superpathway of glycolysis and Entner-Doudoroff |
| GLYCOLYSIS-TCA-GLYOX- | superpathway of glycolysis, pyruvate dehydrogenase, |
| BYPASS | TCA, and glyoxylate bypass |
| GLYOXYLATE-BYPASS | glyoxylate cycle |
| GOLPDLCAT-PWY | superpathway of glycerol degradation to 1,3-propanediol |
| HCAMHPDEG-PWY | 3-phenylpropanoate and 3-(3-hydroxyphenyl)propanoate |
| | degradation to 2-oxopent-4-enoate |
| HEME-BIOSYNTHESIS-II | heme biosynthesis I (aerobic) |
| HEMESYN2-PWY | heme biosynthesis II (anaerobic) |
| HISDEG-PWY | L-histidine degradation I |
| HISTSYN-PWY | L-histidine biosynthesis |
| ILEUSYN-PWY | L-isoleucine biosynthesis I (from threonine) |
| KDO-NAGLIPASYN-PWY | superpathway of (Kdo)2-lipid A biosynthesis |
| LACTOSECAT-PWY | lactose and galactose degradation I |
| LEU-DEG2-PWY | L-leucine degradation I |
| METH-ACETATE-PWY | methanogenesis from acetate |
| METHANOGENESIS-PWY | methanogenesis from H2 and CO2 |
| METHGLYUT-PWY | superpathway of methylglyoxal degradation |
| METHYLGALLATE- | methylgallate degradation |
| DEGRADATION-PWY | |
| NONMEVIPP-PWY | methylerythritol phosphate pathway I |
| NONOXIPENT-PWY | pentose phosphate pathway (non-oxidative branch) |
| OANTIGEN-PWY | O-antigen building blocks biosynthesis (E. coli) |
| ORNARGDEG-PWY | superpathway of L-arginine and L-ornithine degradation |
| ORNDEG-PWY | superpathway of ornithine degradation |
| P122-PWY | heterolactic fermentation |
| P124-PWY | Bifidobacterium shunt |
| P125-PWY | superpathway of (R,R)-butanediol biosynthesis |
| P161-PWY | acetylene degradation |
| P162-PWY | L-glutamate degradation V (via hydroxyglutarate) |
| P163-PWY | L-lysine fermentation to acetate and butanoate |
| P164-PWY | purine nucleobases degradation I (anaerobic) |
| P221-PWY | octane oxidation |
| P23-PWY | reductive TCA cycle I |
| P241-PWY | coenzyme B biosynthesis |
| P261-PWY | coenzyme M biosynthesis I |
| P341-PWY | glycolysis V (Pyrococcus) |
| P381-PWY | adenosylcobalamin biosynthesis II (late cobalt |
| | incorporation) |
| P42-PWY | incomplete reductive TCA cycle |
| P441-PWY | superpathway of N-acetylneuraminate degradation |
| P461-PWY | hexitol fermentation to lactate, formate, ethanol and |
| | acetate |
| P4-PWY | superpathway of L-lysine, L-threonine and L-methionine |
| | biosynthesis I |
| P562-PWY | myo-inositol degradation I |
| PANTO-PWY | phosphopantothenate biosynthesis I |
| PANTOSYN-PWY | pantothenate and coenzyme A biosynthesis I |
| PENTOSE-P-PWY | pentose phosphate pathway |
| PEPTIDOGLYCANSYN-PWY | peptidoglycan biosynthesis I (meso-diaminopimelate |
| | containing) |
| PHOSLIPSYN-PWY | superpathway of phospholipid biosynthesis I (bacteria) |
| POLYAMINSYN3-PWY | superpathway of polyamine biosynthesis II |
| POLYISOPRENSYN-PWY | polyisoprenoid biosynthesis (E. coli) |
| PPGPPMET-PWY | ppGpp biosynthesis |
| PROTOCATECHUATE- | protocatechuate degradation II (ortho-cleavage pathway) |
| ORTHO-CLEAVAGE-PWY | |
| PWY0-1241 | ADP-L-glycero-&beta;-D-manno-heptose biosynthesis |
| PWY0-1261 | anhydromuropeptides recycling |
| PWY0-1277 | 3-phenylpropanoate and 3-(3-hydroxyphenyl)propanoate |
| | degradation |
| PWY0-1296 | purine ribonucleosides degradation |
| PWY0-1297 | superpathway of purine deoxyribonucleosides |
| | degradation |
| PWY0-1298 | superpathway of pyrimidine deoxyribonucleosides |
| | degradation |
| PWY0-1319 | CDP-diacylglycerol biosynthesis II |
| PWY0-1338 | polymyxin resistance |
| PWY0-1415 | superpathway of heme biosynthesis from |
| | uroporphyrinogen-III |
| PWY0-1533 | methylphosphonate degradation I |
| PWY0-162 | superpathway of pyrimidine ribonucleotides de novo |
| | biosynthesis |
| PWY0-166 | superpathway of pyrimidine deoxyribonucleotides de |
| | novo biosynthesis (E. coli) |
| PWY0-321 | phenylacetate degradation I (aerobic) |
| PWY0-41 | allantoin degradation IV (anaerobic) |
| PWY0-781 | aspartate superpathway |
| PWY0-845 | superpathway of pyridoxal 5′-phosphate biosynthesis and |
| | salvage |
| PWY0-862 | (5Z)-dodec-5-enoate biosynthesis |
| PWY-181 | photorespiration |
| PWY-1861 | formaldehyde assimilation II (RuMP Cycle) |
| PWY-2941 | L-lysine biosynthesis II |
| PWY-2942 | L-lysine biosynthesis III |
| PWY-3001 | superpathway of L-isoleucine biosynthesis I |
| PWY-3781 | aerobic respiration I (cytochrome c) |
| PWY-3801 | sucrose degradation II (sucrose synthase) |
| PWY-4722 | creatinine degradation II |
| PWY490-3 | nitrate reduction VI (assimilatory) |
| PWY-4984 | urea cycle |
| PWY4FS-7 | phosphatidylglycerol biosynthesis I (plastidic) |
| PWY4FS-8 | phosphatidylglycerol biosynthesis II (non-plastidic) |
| PWY-5005 | biotin biosynthesis II |
| PWY-5022 | 4-aminobutanoate degradation V |
| PWY-5028 | L-histidine degradation II |
| PWY-5088 | L-glutamate degradation VIII (to propanoate) |
| PWY-5097 | L-lysine biosynthesis VI |
| PWY-5100 | pyruvate fermentation to acetate and lactate II |
| PWY-5101 | L-isoleucine biosynthesis II |
| PWY-5103 | L-isoleucine biosynthesis III |
| PWY-5104 | L-isoleucine biosynthesis IV |
| PWY-5121 | superpathway of geranylgeranyl diphosphate |
| | biosynthesis II (via MEP) |
| PWY-5154 | L-arginine biosynthesis III (via N-acetyl-L-citrulline) |
| PWY-5178 | toluene degradation IV (aerobic) (via catechol) |
| PWY-5180 | toluene degradation I (aerobic) (via o-cresol) |
| PWY-5181 | toluene degradation III (aerobic) (via p-cresol) |
| PWY-5188 | tetrapyrrole biosynthesis I (from glutamate) |
| PWY-5189 | tetrapyrrole biosynthesis II (from glycine) |
| PWY-5198 | factor 420 biosynthesis |
| PWY-5265 | peptidoglycan biosynthesis II (staphylococci) |
| PWY-5304 | superpathway of sulfur oxidation (Acidianus ambivalens) |
| PWY-5347 | superpathway of L-methionine biosynthesis |
| | (transsulfuration) |
| PWY-5384 | sucrose degradation IV (sucrose phosphorylase) |
| PWY-5484 | glycolysis II (from fructose 6-phosphate) |
| PWY-5505 | L-glutamate and L-glutamine biosynthesis |
| PWY-5507 | adenosylcobalamin biosynthesis I (early cobalt insertion) |
| PWY-5509 | adenosylcobalamin biosynthesis from cobyrinate a,c- |
| | diamide I |
| PWY-5531 | chlorophyllide a biosynthesis II (anaerobic) |
| PWY-5659 | GDP-mannose biosynthesis |
| PWY-5667 | CDP-diacylglycerol biosynthesis I |
| PWY-5676 | acetyl-CoA fermentation to butanoate II |
| PWY-5686 | UMP biosynthesis |
| PWY-5695 | urate biosynthesis/inosine 5′-phosphate degradation |
| PWY-5705 | allantoin degradation to glyoxylate III |
| PWY-5837 | 1,4-dihydroxy-2-naphthoate biosynthesis I |
| PWY-5838 | superpathway of menaquinol-8 biosynthesis I |
| PWY-5840 | superpathway of menaquinol-7 biosynthesis |
| PWY-5845 | superpathway of menaquinol-9 biosynthesis |
| PWY-5850 | superpathway of menaquinol-6 biosynthesis I |
| PWY-5860 | superpathway of demethylmenaquinol-6 biosynthesis I |
| PWY-5861 | superpathway of demethylmenaquinol-8 biosynthesis |
| PWY-5862 | superpathway of demethylmenaquinol-9 biosynthesis |
| PWY-5863 | superpathway of phylloquinol biosynthesis |
| PWY-5896 | superpathway of menaquinol-10 biosynthesis |
| PWY-5897 | superpathway of menaquinol-11 biosynthesis |
| PWY-5898 | superpathway of menaquinol-12 biosynthesis |
| PWY-5899 | superpathway of menaquinol-13 biosynthesis |
| PWY-5910 | superpathway of geranylgeranyldiphosphate biosynthesis |
| | I (via mevalonate) |
| PWY-5913 | TCA cycle VI (obligate autotrophs) |
| PWY-5971 | palmitate biosynthesis II (bacteria and plants) |
| PWY-5973 | cis-vaccenate biosynthesis |
| PWY-6071 | superpathway of phenylethylamine degradation |
| PWY-6121 | 5-aminoimidazole ribonucleotide biosynthesis I |
| PWY-6122 | 5-aminoimidazole ribonucleotide biosynthesis II |
| PWY-6123 | inosine-5′-phosphate biosynthesis I |
| PWY-6125 | superpathway of guanosine nucleotides de novo |
| | biosynthesis II |
| PWY-6126 | superpathway of adenosine nucleotides de novo |
| | biosynthesis II |
| PWY-6141 | archaetidylserine and archaetidylethanolamine |
| | biosynthesis |
| PWY-6147 | 6-hydroxymethyl-dihydropterin diphosphate biosynthesis I |
| PWY-6148 | tetrahydromethanopterin biosynthesis |
| PWY-6151 | S-adenosyl-L-methionine cycle I |
| PWY-6163 | chorismate biosynthesis from 3-dehydroquinate |
| PWY-6165 | chorismate biosynthesis II (archaea) |
| PWY-6167 | flavin biosynthesis II (archaea) |
| PWY-6174 | mevalonate pathway II (archaea) |
| PWY-621 | sucrose degradation III (sucrose invertase) |
| PWY-6263 | superpathway of menaquinol-8 biosynthesis II |
| PWY-6269 | adenosylcobalamin salvage from cobinamide II |
| PWY-6277 | superpathway of 5-aminoimidazole ribonucleotide |
| | biosynthesis |
| PWY-6317 | galactose degradation I (Leloir pathway) |
| PWY-6349 | CDP-archaeol biosynthesis |
| PWY-6350 | archaetidylinositol biosynthesis |
| PWY-6353 | purine nucleotides degradation II (aerobic) |
| PWY-6385 | peptidoglycan biosynthesis III (mycobacteria) |
| PWY-6386 | UDP-N-acetylmuramoyl-pentapeptide biosynthesis II |
| | (lysine-containing) |
| PWY-6387 | UDP-N-acetylmuramoyl-pentapeptide biosynthesis I |
| | (meso-diaminopimelate containing) |
| PWY-6396 | superpathway of 2,3-butanediol biosynthesis |
| PWY-6470 | peptidoglycan biosynthesis V (&beta;-lactam resistance) |
| PWY-6471 | peptidoglycan biosynthesis IV (Enterococcus faecium) |
| PWY-6478 | GDP-D-glycero-&alpha;-D-manno-heptose biosynthesis |
| PWY-6507 | 4-deoxy-L-threo-hex-4-enopyranuronate degradation |
| PWY-6545 | pyrimidine deoxyribonucleotides de novo biosynthesis III |
| PWY-6572 | chondroitin sulfate degradation I (bacterial) |
| PWY-6588 | pyruvate fermentation to acetone |
| PWY-6590 | superpathway of Clostridium acetobutylicum acidogenic |
| | fermentation |
| PWY-6608 | guanosine nucleotides degradation III |
| PWY-6609 | adenine and adenosine salvage III |
| PWY-6612 | superpathway of tetrahydrofolate biosynthesis |
| PWY-6641 | superpathway of sulfolactate degradation |
| PWY-6654 | phosphopantothenate biosynthesis III |
| PWY-6690 | cinnamate and 3-hydroxycinnamate degradation to 2- |
| | oxopent-4-enoate |
| PWY-6700 | queuosine biosynthesis |
| PWY-6703 | preQ0 biosynthesis |
| PWY-6737 | starch degradation V |
| PWY-6749 | CMP-legionaminate biosynthesis I |
| PWY-6876 | isopropanol biosynthesis |
| PWY-6891 | thiazole biosynthesis II (Bacillus) |
| PWY-6892 | thiazole biosynthesis I (E. coli) |
| PWY-6895 | superpathway of thiamin diphosphate biosynthesis II |
| PWY-6897 | thiamin salvage II |
| PWY-6901 | superpathway of glucose and xylose degradation |
| PWY-6906 | chitin derivatives degradation |
| PWY-6969 | TCA cycle V (2-oxoglutarate:ferredoxin oxidoreductase) |
| PWY-7003 | glycerol degradation to butanol |
| PWY-7013 | L-1,2-propanediol degradation |
| PWY-7090 | UDP-2,3-diacetamido-2,3-dideoxy-&alpha;-D-mannuronate biosynthesis |
| PWY-7094 | fatty acid salvage |
| PWY-7111 | pyruvate fermentation to isobutanol (engineered) |
| PWY-7159 | chlorophyllide a biosynthesis III (aerobic, light |
| | independent) |
| PWY-7184 | pyrimidine deoxyribonucleotides de novo biosynthesis I |
| PWY-7187 | pyrimidine deoxyribonucleotides de novo biosynthesis II |
| PWY-7196 | superpathway of pyrimidine ribonucleosides salvage |
| PWY-7197 | pyrimidine deoxyribonucleotide phosphorylation |
| PWY-7198 | pyrimidine deoxyribonucleotides de novo biosynthesis IV |
| PWY-7199 | pyrimidine deoxyribonucleosides salvage |
| PWY-7200 | superpathway of pyrimidine deoxyribonucleoside salvage |
| PWY-7208 | superpathway of pyrimidine nucleobases salvage |
| PWY-7209 | superpathway of pyrimidine ribonucleosides degradation |
| PWY-7210 | pyrimidine deoxyribonucleotides biosynthesis from CTP |
| PWY-7219 | adenosine ribonucleotides de novo biosynthesis |
| PWY-7220 | adenosine deoxyribonucleotides de novo biosynthesis II |
| PWY-7221 | guanosine ribonucleotides de novo biosynthesis |
| PWY-7222 | guanosine deoxyribonucleotides de novo biosynthesis II |
| PWY-7228 | superpathway of guanosine nucleotides de novo |
| | biosynthesis I |
| PWY-7229 | superpathway of adenosine nucleotides de novo |
| | biosynthesis I |
| PWY-7234 | inosine-5′-phosphate biosynthesis III |
| PWY-7237 | myo-, chiro- and scillo-inositol degradation |
| PWY-7242 | D-fructuronate degradation |
| PWY-7286 | 7-(3-amino-3-carboxypropyl)-wyosine biosynthesis |
| PWY-7315 | dTDP-N-acetylthomosamine biosynthesis |
| PWY-7328 | superpathway of UDP-glucose-derived O-antigen |
| | building blocks biosynthesis |
| PWY-7332 | superpathway of UDP-N-acetylglucosamine-derived O- |
| | antigen building blocks biosynthesis |
| PWY-7371 | 1,4-dihydroxy-6-naphthoate biosynthesis II |
| PWY-7374 | 1,4-dihydroxy-6-naphthoate biosynthesis I |
| PWY-7376 | cob(II)yrinate a,c-diamide biosynthesis II (late cobalt |
| | incorporation) |
| PWY-7377 | cob(II)yrinate a,c-diamide biosynthesis I (early cobalt |
| | insertion) |
| PWY-7391 | isoprene biosynthesis II (engineered) |
| PWY-7392 | taxadiene biosynthesis (engineered) |
| PWY-7400 | L-arginine biosynthesis IV (archaebacteria) |
| PWY-7431 | aromatic biogenic amine degradation (bacteria) |
| PWY-7456 | mannan degradation |
| PWY-7539 | 6-hydroxymethyl-dihydropterin diphosphate biosynthesis |
| | III (Chlamydia) |
| PWY-7560 | methylerythritol phosphate pathway II |
| PWY-7663 | gondoate biosynthesis (anaerobic) |
| PWY-841 | superpathway of purine nucleotides de novo biosynthesis I |
| PWY-922 | mevalonate pathway I |
| PWYG-321 | mycolate biosynthesis |
| PYRIDNUCSAL-PWY | NAD salvage pathway I |
| PYRIDNUCSYN-PWY | NAD biosynthesis I (from aspartate) |
| PYRIDOXSYN-PWY | pyridoxal 5′-phosphate biosynthesis I |
| REDCITCYC | TCA cycle VIII (helicobacter) |
| RHAMCAT-PWY | L-rhamnose degradation I |
| RIBOSYN2-PWY | flavin biosynthesis I (bacteria and plants) |
| SALVADEHYPOX-PWY | adenosine nucleotides degradation II |
| SER-GLYSYN-PWY | superpathway of L-serine and glycine biosynthesis I |
| SO4ASSIM-PWY | sulfate reduction I (assimilatory) |
| SULFATE-CYS-PWY | superpathway of sulfate assimilation and cysteine |
| | biosynthesis |
| TCA | TCA cycle I (prokaryotic) |
| TCA-GLYOX-BYPASS | superpathway of glyoxylate bypass and TCA |
| THISYN-PWY | superpathway of thiamin diphosphate biosynthesis I |
| THREOCAT-PWY | superpathway of L-threonine metabolism |
| THRESYN-PWY | superpathway of L-threonine biosynthesis |
| TRNA-CHARGING-PWY | tRNA charging |
| TRPSYN-PWY | L-tryptophan biosynthesis |
| TYRFUMCAT-PWY | L-tyrosine degradation I |
| UDPNAGSYN-PWY | UDP-N-acetyl-D-glucosamine biosynthesis I |
| VALDEG-PWY | L-valine degradation I |
| VALSYN-PWY | L-valine biosynthesis |

TABLE 19
Taxa4Meta taxonomic classifiers identified as features
Feature (taxonomic order: kingdom; phylum;
class; order; family; genus; species)

Archaea; Euryarchaeota; Methanobacteria; Methanobacteriales; Methanobacteriaceae; Methanobrevibacter;
Methanobrevibacter smithii
Archaea; Euryarchaeota; Methanobacteria; Methanobacteriales; Methanobacteriaceae; Methanobrevibacter;
Other
Bacteria; Actinobacteria; Actinobacteria; Actinomycetales; Actinomycetaceae; Actinomyces; Other
Bacteria; Actinobacteria; Actinobacteria; Actinomycetales; Actinomycetaceae; Other; Other
Bacteria; Actinobacteria; Actinobacteria; Actinomycetales; Actinomycetaceae; Schaalia;
Schaalia odontolytica
Bacteria; Actinobacteria; Actinobacteria; Bifidobacteriales; Bifidobacteriaceae; Bifidobacterium;
Bifidobacterium adolescentis
Bacteria; Actinobacteria; Actinobacteria; Bifidobacteriales; Bifidobacteriaceae; Bifidobacterium;
Bifidobacterium bifidum
Bacteria; Actinobacteria; Actinobacteria; Bifidobacteriales; Bifidobacteriaceae; Bifidobacterium;
Bifidobacterium boum
Bacteria; Actinobacteria; Actinobacteria; Bifidobacteriales; Bifidobacteriaceae; Bifidobacterium;
Bifidobacterium longum
Bacteria; Actinobacteria; Actinobacteria; Bifidobacteriales; Bifidobacteriaceae; Bifidobacterium;
Other
Bacteria; Actinobacteria; Actinobacteria; Bifidobacteriales; Bifidobacteriaceae; Other; Other
Bacteria; Actinobacteria; Actinobacteria; Coriobacteriales; Coriobacteriaceae; Collinsella; Other
Bacteria; Actinobacteria; Actinobacteria; Coriobacteriales; Coriobacteriaceae; Eggerthella; Other
Bacteria; Actinobacteria; Actinobacteria; Coriobacteriales; Coriobacteriaceae; Other; Other
Bacteria; Actinobacteria; Actinobacteria; Other; Other; Other; Other
Bacteria; Actinobacteria; Coriobacteriia; Coriobacteriales; Coriobacteriaceae; Collinsella;
Collinsella aerofaciens
Bacteria; Actinobacteria; Coriobacteriia; Eggerthellales; Eggerthellaceae; Adlercreutzia;
Adlercreutzia equolifaciens
Bacteria; Actinobacteria; Coriobacteriia; Eggerthellales; Eggerthellaceae; Eggerthella; Eggerthella
lenta
Bacteria; Actinobacteria; Coriobacteriia; Eggerthellales; Eggerthellaceae; Gordonibacter;
Gordonibacter pamelaeae
Bacteria; Actinobacteria; Coriobacteriia; Eggerthellales; Eggerthellaceae; Other; Other
Bacteria; Bacteroidetes; Bacteroidia; Bacteroidales; Bacteroidaceae; Bacteroides; Bacteroides
acidifaciens
Bacteria; Bacteroidetes; Bacteroidia; Bacteroidales; Bacteroidaceae; Bacteroides; Bacteroides
caccae
Bacteria; Bacteroidetes; Bacteroidia; Bacteroidales; Bacteroidaceae; Bacteroides; Bacteroides
cellulosilyticus
Bacteria; Bacteroidetes; Bacteroidia; Bacteroidales; Bacteroidaceae; Bacteroides; Bacteroides
coprocola
Bacteria; Bacteroidetes; Bacteroidia; Bacteroidales; Bacteroidaceae; Bacteroides; Bacteroides
coprophilus
Bacteria; Bacteroidetes; Bacteroidia; Bacteroidales; Bacteroidaceae; Bacteroides; Bacteroides
dorei
Bacteria; Bacteroidetes; Bacteroidia; Bacteroidales; Bacteroidaceae; Bacteroides; Bacteroides
eggerthii
Bacteria; Bacteroidetes; Bacteroidia; Bacteroidales; Bacteroidaceae; Bacteroides; Bacteroides
finegoldii
Bacteria; Bacteroidetes; Bacteroidia; Bacteroidales; Bacteroidaceae; Bacteroides; Bacteroides
koreensis
Bacteria; Bacteroidetes; Bacteroidia; Bacteroidales; Bacteroidaceae; Bacteroides; Bacteroides
massiliensis
Bacteria; Bacteroidetes; Bacteroidia; Bacteroidales; Bacteroidaceae; Bacteroides; Bacteroides
nordii
Bacteria; Bacteroidetes; Bacteroidia; Bacteroidales; Bacteroidaceae; Bacteroides; Bacteroides
ovatus
Bacteria; Bacteroidetes; Bacteroidia; Bacteroidales; Bacteroidaceae; Bacteroides; Bacteroides
plebeius
Bacteria; Bacteroidetes; Bacteroidia; Bacteroidales; Bacteroidaceae; Bacteroides; Bacteroides
thetaiotaomicron
Bacteria; Bacteroidetes; Bacteroidia; Bacteroidales; Bacteroidaceae; Bacteroides; Bacteroides
uniformis
Bacteria; Bacteroidetes; Bacteroidia; Bacteroidales; Bacteroidaceae; Bacteroides; Bacteroides
vulgatus
Bacteria; Bacteroidetes; Bacteroidia; Bacteroidales; Bacteroidaceae; Bacteroides; Bacteroides
xylanisolvens
Bacteria; Bacteroidetes; Bacteroidia; Bacteroidales; Bacteroidaceae; Bacteroides; Other
Bacteria; Bacteroidetes; Bacteroidia; Bacteroidales; Barnesiellaceae; Coprobacter; Coprobacter
fastidiosus
Bacteria; Bacteroidetes; Bacteroidia; Bacteroidales; Odoribacteraceae; Odoribacter; Odoribacter
splanchnicus
Bacteria; Bacteroidetes; Bacteroidia; Bacteroidales; Other; Other; Other
Bacteria; Bacteroidetes; Bacteroidia; Bacteroidales; Porphyromonadaceae; Barnesiella; Other
Bacteria; Bacteroidetes; Bacteroidia; Bacteroidales; Porphyromonadaceae; Butyricimonas; Other
Bacteria; Bacteroidetes; Bacteroidia; Bacteroidales; Porphyromonadaceae; Odoribacter; Other
Bacteria; Bacteroidetes; Bacteroidia; Bacteroidales; Porphyromonadaceae; Other; Other
Bacteria; Bacteroidetes; Bacteroidia; Bacteroidales; Porphyromonadaceae; Parabacteroides; Other
Bacteria; Bacteroidetes; Bacteroidia; Bacteroidales; Prevotellaceae; Paraprevotella; Other
Bacteria; Bacteroidetes; Bacteroidia; Bacteroidales; Prevotellaceae; Prevotella; Other
Bacteria; Bacteroidetes; Bacteroidia; Bacteroidales; Prevotellaceae; Prevotella; Prevotella copri
Bacteria; Bacteroidetes; Bacteroidia; Bacteroidales; Prevotellaceae; Prevotellamassilia;
Prevotellamassilia timonensis
Bacteria; Bacteroidetes; Bacteroidia; Bacteroidales; Rikenellaceae; Alistipes; Alistipes finegoldii
Bacteria; Bacteroidetes; Bacteroidia; Bacteroidales; Rikenellaceae; Alistipes; Alistipes ihumii
Bacteria; Bacteroidetes; Bacteroidia; Bacteroidales; Rikenellaceae; Alistipes; Alistipes obesi
Bacteria; Bacteroidetes; Bacteroidia; Bacteroidales; Rikenellaceae; Alistipes; Alistipes
onderdonkii
Bacteria; Bacteroidetes; Bacteroidia; Bacteroidales; Rikenellaceae; Alistipes; Alistipes putredinis
Bacteria; Bacteroidetes; Bacteroidia; Bacteroidales; Rikenellaceae; Alistipes; Alistipes shahii
Bacteria; Bacteroidetes; Bacteroidia; Bacteroidales; Rikenellaceae; Alistipes; Other
Bacteria; Bacteroidetes; Bacteroidia; Bacteroidales; Rikenellaceae; Other; Other
Bacteria; Bacteroidetes; Bacteroidia; Bacteroidales; Rikenellaceae; Tidjanibacter; Tidjanibacter
massiliensis
Bacteria; Bacteroidetes; Bacteroidia; Bacteroidales; Tannerellaceae; Parabacteroides; Parabacteroides
distasonis
Bacteria; Bacteroidetes; Bacteroidia; Bacteroidales; Tannerellaceae; Parabacteroides; Parabacteroides
merdae
Bacteria; Bacteroidetes; Other; Other; Other; Other; Other
Bacteria; Bacteroidetes; Sphingobacteriia; Sphingobacteriales; Sphingobacteriaceae; Pedobacter;
Other
Bacteria; Candidatus Saccharibacteria; Saccharibacteria_genera_incertae_sedis; Other; Other; Other; Other
Bacteria; Cyanobacteria/Chloroplast; Chloroplast; Chloroplast; Streptophyta; Other; Other
Bacteria; Firmicutes; Bacilli; Bacillales; Bacillales_Incertae Sedis XI; Gemella; Other
Bacteria; Firmicutes; Bacilli; Bacillales; Other; Gemella; Other
Bacteria; Firmicutes; Bacilli; Lactobacillales; Carnobacteriaceae; Granulicatella; Granulicatella
adiacens
Bacteria; Firmicutes; Bacilli; Lactobacillales; Carnobacteriaceae; Granulicatella; Other
Bacteria; Firmicutes; Bacilli; Lactobacillales; Enterococcaceae; Enterococcus; Enterococcus
saccharolyticus
Bacteria; Firmicutes; Bacilli; Lactobacillales; Enterococcaceae; Enterococcus; Other
Bacteria; Firmicutes; Bacilli; Lactobacillales; Enterococcaceae; Other; Other
Bacteria; Firmicutes; Bacilli; Lactobacillales; Lactobacillaceae; Lactobacillus; Lactobacillus
rogosae
Bacteria; Firmicutes; Bacilli; Lactobacillales; Lactobacillaceae; Lactobacillus; Lactobacillus sakei
Bacteria; Firmicutes; Bacilli; Lactobacillales; Lactobacillaceae; Lactobacillus; Other
Bacteria; Firmicutes; Bacilli; Lactobacillales; Lactobacillaceae; Leuconostoc; Other
Bacteria; Firmicutes; Bacilli; Lactobacillales; Other; Other; Other
Bacteria; Firmicutes; Bacilli; Lactobacillales; Streptococcaceae; Other; Other
Bacteria; Firmicutes; Bacilli; Lactobacillales; Streptococcaceae; Streptococcus; Other
Bacteria; Firmicutes; Bacilli; Lactobacillales; Streptococcaceae; Streptococcus; Streptococcus
parasanguinis
Bacteria; Firmicutes; Bacilli; Lactobacillales; Streptococcaceae; Streptococcus; Streptococcus
salivarius
Bacteria; Firmicutes; Bacilli; Lactobacillales; Streptococcaceae; Streptococcus; Streptococcus
sanguinis
Bacteria; Firmicutes; Bacilli; Lactobacillales; Streptococcaceae; Streptococcus; Streptococcus
thermophilus
Bacteria; Firmicutes; Bacilli; Other; Other; Other; Other
Bacteria; Firmicutes; Clostridia; Clostridiales; Clostridiaceae 1; Clostridium sensu stricto; Other
Bacteria; Firmicutes; Clostridia; Clostridiales; Clostridiaceae; Clostridium; Clostridium cadaveris
Bacteria; Firmicutes; Clostridia; Clostridiales; Clostridiaceae; Clostridium; Clostridium
paraputrificum
Bacteria; Firmicutes; Clostridia; Clostridiales; Clostridiaceae; Clostridium; Clostridium
perfringens
Bacteria; Firmicutes; Clostridia; Clostridiales; Clostridiaceae; Clostridium; Other
Bacteria; Firmicutes; Clostridia; Clostridiales; Clostridiaceae; Hungatella; Hungatella effluvii
Bacteria; Firmicutes; Clostridia; Clostridiales; Clostridiales Family XIII. Incertae
Sedis; Ihubacter; Ihubacter massiliensis
Bacteria; Firmicutes; Clostridia; Clostridiales; Clostridiales Family XIII. Incertae Sedis; Not
Available; [Eubacterium] sulci
Bacteria; Firmicutes; Clostridia; Clostridiales; Clostridiales_Incertae Sedis XI; Parvimonas; Other
Bacteria; Firmicutes; Clostridia; Clostridiales; Eubacteriaceae; Anaerofustis; Other
Bacteria; Firmicutes; Clostridia; Clostridiales; Eubacteriaceae; Eubacterium; [Eubacterium]
eligens
Bacteria; Firmicutes; Clostridia; Clostridiales; Eubacteriaceae; Eubacterium; Eubacterium
ventriosum
Bacteria; Firmicutes; Clostridia; Clostridiales; Eubacteriaceae; Eubacterium; Other
Bacteria; Firmicutes; Clostridia; Clostridiales; Lachnospiraceae; Anaerostipes; Anaerostipes
hadrus
Bacteria; Firmicutes; Clostridia; Clostridiales; Lachnospiraceae; Anaerostipes; Other
Bacteria; Firmicutes; Clostridia; Clostridiales; Lachnospiraceae; Blautia; [Ruminococcus] gnavus
Bacteria; Firmicutes; Clostridia; Clostridiales; Lachnospiraceae; Blautia; Blautia caecimuris
Bacteria; Firmicutes; Clostridia; Clostridiales; Lachnospiraceae; Blautia; Blautia faecis
Bacteria; Firmicutes; Clostridia; Clostridiales; Lachnospiraceae; Blautia; Blautia hominis
Bacteria; Firmicutes; Clostridia; Clostridiales; Lachnospiraceae; Blautia; Blautia
hydrogenotrophica
Bacteria; Firmicutes; Clostridia; Clostridiales; Lachnospiraceae; Blautia; Blautia luti
Bacteria; Firmicutes; Clostridia; Clostridiales; Lachnospiraceae; Blautia; Blautia obeum
Bacteria; Firmicutes; Clostridia; Clostridiales; Lachnospiraceae; Blautia; Blautia producta
Bacteria; Firmicutes; Clostridia; Clostridiales; Lachnospiraceae; Blautia; Blautia stercoris
Bacteria; Firmicutes; Clostridia; Clostridiales; Lachnospiraceae; Blautia; Blautia wexlerae
Bacteria; Firmicutes; Clostridia; Clostridiales; Lachnospiraceae; Blautia; Other
Bacteria; Firmicutes; Clostridia; Clostridiales; Lachnospiraceae; Clostridium XIVa; Other
Bacteria; Firmicutes; Clostridia; Clostridiales; Lachnospiraceae; Coprococcus; Coprococcus catus
Bacteria; Firmicutes; Clostridia; Clostridiales; Lachnospiraceae; Coprococcus; Coprococcus
comes
Bacteria; Firmicutes; Clostridia; Clostridiales; Lachnospiraceae; Coprococcus; Coprococcus
eutactus
Bacteria; Firmicutes; Clostridia; Clostridiales; Lachnospiraceae; Coprococcus; Other
Bacteria; Firmicutes; Clostridia; Clostridiales; Lachnospiraceae; Dorea; Dorea formicigenerans
Bacteria; Firmicutes; Clostridia; Clostridiales; Lachnospiraceae; Dorea; Dorea longicatena
Bacteria; Firmicutes; Clostridia; Clostridiales; Lachnospiraceae; Dorea; Other
Bacteria; Firmicutes; Clostridia; Clostridiales; Lachnospiraceae; Eisenbergiella; Eisenbergiella
tayi
Bacteria; Firmicutes; Clostridia; Clostridiales; Lachnospiraceae; Faecalicatena; Faecalicatena
fissicatena
Bacteria; Firmicutes; Clostridia; Clostridiales; Lachnospiraceae; Faecalimonas; Faecalimonas
umbilicata
Bacteria; Firmicutes; Clostridia; Clostridiales; Lachnospiraceae; Fusicatenibacter; Fusicatenibacter
saccharivorans
Bacteria; Firmicutes; Clostridia; Clostridiales; Lachnospiraceae; Fusicatenibacter; Other
Bacteria; Firmicutes; Clostridia; Clostridiales; Lachnospiraceae; Hungatella; Other
Bacteria; Firmicutes; Clostridia; Clostridiales; Lachnospiraceae; Lachnoclostridium; [Clostridium]
aldenense
Bacteria; Firmicutes; Clostridia; Clostridiales; Lachnospiraceae; Lachnoclostridium; [Clostridium]
bolteae
Bacteria; Firmicutes; Clostridia; Clostridiales; Lachnospiraceae; Lachnoclostridium; [Clostridium]
citroniae
Bacteria; Firmicutes; Clostridia; Clostridiales; Lachnospiraceae; Lachnoclostridium; [Clostridium]
glycyrrhizinilyticum
Bacteria; Firmicutes; Clostridia; Clostridiales; Lachnospiraceae; Lachnoclostridium; [Clostridium]
lavalense
Bacteria; Firmicutes; Clostridia; Clostridiales; Lachnospiraceae; Lachnoclostridium; Other
Bacteria; Firmicutes; Clostridia; Clostridiales; Lachnospiraceae; Lachnospiracea_incertae_sedis;
Other
Bacteria; Firmicutes; Clostridia; Clostridiales; Lachnospiraceae; Not Available; [Eubacterium]
rectale
Bacteria; Firmicutes; Clostridia; Clostridiales; Lachnospiraceae; Other; Other
Bacteria; Firmicutes; Clostridia; Clostridiales; Lachnospiraceae; Roseburia; Other
Bacteria; Firmicutes; Clostridia; Clostridiales; Lachnospiraceae; Roseburia; Roseburia faecis
Bacteria; Firmicutes; Clostridia; Clostridiales; Lachnospiraceae; Roseburia; Roseburia hominis
Bacteria; Firmicutes; Clostridia; Clostridiales; Lachnospiraceae; Roseburia; Roseburia intestinalis
Bacteria; Firmicutes; Clostridia; Clostridiales; Lachnospiraceae; Roseburia; Roseburia
inulinivorans
Bacteria; Firmicutes; Clostridia; Clostridiales; Lachnospiraceae; Ruminococcus2; Other
Bacteria; Firmicutes; Clostridia; Clostridiales; Lachnospiraceae; Tyzzerella; Tyzzerella nexilis
Bacteria; Firmicutes; Clostridia; Clostridiales; Not Available; Colidextribacter; Colidextribacter
massiliensis
Bacteria; Firmicutes; Clostridia; Clostridiales; Not Available; Intestinimonas; Intestinimonas
butyriciproducens
Bacteria; Firmicutes; Clostridia; Clostridiales; Other; Other; Other
Bacteria; Firmicutes; Clostridia; Clostridiales; Peptostreptococcaceae; Clostridioides; Clostridioides
difficile
Bacteria; Firmicutes; Clostridia; Clostridiales; Peptostreptococcaceae; Clostridium XI; Other
Bacteria; Firmicutes; Clostridia; Clostridiales; Peptostreptococcaceae; Intestinibacter;
Intestinibacter bartlettii
Bacteria; Firmicutes; Clostridia; Clostridiales; Peptostreptococcaceae; Intestinibacter; Other
Bacteria; Firmicutes; Clostridia; Clostridiales; Peptostreptococcaceae; Other; Other
Bacteria; Firmicutes; Clostridia; Clostridiales; Peptostreptococcaceae; Peptostreptococcus; Other
Bacteria; Firmicutes; Clostridia; Clostridiales; Peptostreptococcaceae; Romboutsia; Other
Bacteria; Firmicutes; Clostridia; Clostridiales; Peptostreptococcaceae; Romboutsia; Romboutsia
timonensis
Bacteria; Firmicutes; Clostridia; Clostridiales; Peptostreptococcaceae; Terrisporobacter; Other
Bacteria; Firmicutes; Clostridia; Clostridiales; Ruminococcaceae; Agathobaculum; Agathobaculum
butyriciproducens
Bacteria; Firmicutes; Clostridia; Clostridiales; Ruminococcaceae; Anaeromassilibacillus;
Anaeromassilibacillus senegalensis
Bacteria; Firmicutes; Clostridia; Clostridiales; Ruminococcaceae; Anaerotruncus; Anaerotruncus
colihominis
Bacteria; Firmicutes; Clostridia; Clostridiales; Ruminococcaceae; Anaerotruncus; Other
Bacteria; Firmicutes; Clostridia; Clostridiales; Ruminococcaceae; Clostridium IV; Other
Bacteria; Firmicutes; Clostridia; Clostridiales; Ruminococcaceae; Faecalibacterium; Faecalibacterium
prausnitzii
Bacteria; Firmicutes; Clostridia; Clostridiales; Ruminococcaceae; Faecalibacterium; Other
Bacteria; Firmicutes; Clostridia; Clostridiales; Ruminococcaceae; Flavonifractor; Flavonifractor
plautii
Bacteria; Firmicutes; Clostridia; Clostridiales; Ruminococcaceae; Flavonifractor; Other
Bacteria; Firmicutes; Clostridia; Clostridiales; Ruminococcaceae; Gemmiger; Gemmiger
formicilis
Bacteria; Firmicutes; Clostridia; Clostridiales; Ruminococcaceae; Gemmiger; Other
Bacteria; Firmicutes; Clostridia; Clostridiales; Ruminococcaceae; Intestinimonas; Other
Bacteria; Firmicutes; Clostridia; Clostridiales; Ruminococcaceae; Negativibacillus; Negativibacillus
massiliensis
Bacteria; Firmicutes; Clostridia; Clostridiales; Ruminococcaceae; Neglecta; Neglecta timonensis
Bacteria; Firmicutes; Clostridia; Clostridiales; Ruminococcaceae; Not Available; [Clostridium]
leptum
Bacteria; Firmicutes; Clostridia; Clostridiales; Ruminococcaceae; Not Available; [Eubacterium]
siraeum
Bacteria; Firmicutes; Clostridia; Clostridiales; Ruminococcaceae; Oscillibacter; Other
Bacteria; Firmicutes; Clostridia; Clostridiales; Ruminococcaceae; Other; Other
Bacteria; Firmicutes; Clostridia; Clostridiales; Ruminococcaceae; Pseudoflavonifractor; Other
Bacteria; Firmicutes; Clostridia; Clostridiales; Ruminococcaceae; Pseudoflavonifractor;
Pseudoflavonifractor capillosus
Bacteria; Firmicutes; Clostridia; Clostridiales; Ruminococcaceae; Ruminococcus; Other
Bacteria; Firmicutes; Clostridia; Clostridiales; Ruminococcaceae; Ruminococcus; Ruminococcus
bromii
Bacteria; Firmicutes; Clostridia; Clostridiales; Ruminococcaceae; Ruminococcus; Ruminococcus
callidus
Bacteria; Firmicutes; Clostridia; Clostridiales; Ruminococcaceae; Ruminococcus; Ruminococcus
faecis
Bacteria; Firmicutes; Clostridia; Clostridiales; Ruminococcaceae; Ruminococcus; Ruminococcus
lactaris
Bacteria; Firmicutes; Clostridia; Clostridiales; Ruminococcaceae; Ruthenibacterium; Ruthenibacterium
lactatiformans
Bacteria; Firmicutes; Clostridia; Clostridiales; Ruminococcaceae; Subdoligranulum; Other
Bacteria; Firmicutes; Clostridia; Other; Other; Other; Other
Bacteria; Firmicutes; Erysipelotrichia; Erysipelotrichales; Erysipelotrichaceae; Clostridium
XVIII; Other
Bacteria; Firmicutes; Erysipelotrichia; Erysipelotrichales; Erysipelotrichaceae; Erysipelatoclostridium;
[Clostridium] innocuum
Bacteria; Firmicutes; Erysipelotrichia; Erysipelotrichales; Erysipelotrichaceae; Erysipelatoclostridium;
[Clostridium] spiroforme
Bacteria; Firmicutes; Erysipelotrichia; Erysipelotrichales; Erysipelotrichaceae; Erysipelatoclostridium;
Erysipelatoclostridium ramosum
Bacteria; Firmicutes; Erysipelotrichia; Erysipelotrichales; Erysipelotrichaceae; Faecalicoccus; Other
Bacteria; Firmicutes; Erysipelotrichia; Erysipelotrichales; Erysipelotrichaceae; Holdemania; Holdemania
filiformis
Bacteria; Firmicutes; Erysipelotrichia; Erysipelotrichales; Erysipelotrichaceae; Holdemania; Other
Bacteria; Firmicutes; Erysipelotrichia; Erysipelotrichales; Erysipelotrichaceae; Massilimicrobiota;
Massilimicrobiota timonensis
Bacteria; Firmicutes; Erysipelotrichia; Erysipelotrichales; Erysipelotrichaceae; Other; Other
Bacteria; Firmicutes; Erysipelotrichia; Erysipelotrichales; Erysipelotrichaceae; Solobacterium;
Solobacterium moorei
Bacteria; Firmicutes; Erysipelotrichia; Erysipelotrichales; Erysipelotrichaceae; Turicibacter; Other
Bacteria; Firmicutes; Erysipelotrichia; Erysipelotrichales; Erysipelotrichaceae; Turicibacter;
Turicibacter sanguinis
Bacteria; Firmicutes; Negativicutes; Acidaminococcales; Acidaminococcaceae; Phascolarctobacterium;
Phascolarctobacterium faecium
Bacteria; Firmicutes; Negativicutes; Selenomonadales; Acidaminococcaceae; Acidaminococcus;
Other
Bacteria; Firmicutes; Negativicutes; Selenomonadales; Acidaminococcaceae; Phascolarctobacterium;
Other
Bacteria; Firmicutes; Negativicutes; Selenomonadales; Veillonellaceae; Dialister; Other
Bacteria; Firmicutes; Negativicutes; Selenomonadales; Veillonellaceae; Other; Other
Bacteria; Firmicutes; Negativicutes; Selenomonadales; Veillonellaceae; Veillonella; Other
Bacteria; Firmicutes; Negativicutes; Veillonellales; Veillonellaceae; Dialister; Dialister invisus
Bacteria; Firmicutes; Negativicutes; Veillonellales; Veillonellaceae; Megasphaera; Megasphaera
micronuciformis
Bacteria; Firmicutes; Negativicutes; Veillonellales; Veillonellaceae; Veillonella; Other
Bacteria; Firmicutes; Negativicutes; Veillonellales; Veillonellaceae; Veillonella; Veillonella
dispar
Bacteria; Firmicutes; Negativicutes; Veillonellales; Veillonellaceae; Veillonella; Veillonella
infantium
Bacteria; Firmicutes; Negativicutes; Veillonellales; Veillonellaceae; Veillonella; Veillonella
parvula
Bacteria; Firmicutes; Other; Other; Other; Other; Other
Bacteria; Firmicutes; Tissierellia; Tissierellales; Peptoniphilaceae; Peptoniphilus; Other
Bacteria; Fusobacteria; Fusobacteriia; Fusobacteriales; Fusobacteriaceae; Fusobacterium;
Fusobacterium nucleatum
Bacteria; Fusobacteria; Fusobacteriia; Fusobacteriales; Fusobacteriaceae; Fusobacterium; Other
Bacteria; Lentisphaerae; Lentisphaeria; Victivallales; Victivallaceae; Victivallis; Other
Bacteria; Other; Other; Other; Other; Other; Other
Bacteria; Proteobacteria; Alphaproteobacteria; Caulobacterales; Caulobacteraceae; Caulobacter;
Caulobacter segnis
Bacteria; Proteobacteria; Alphaproteobacteria; Caulobacterales; Caulobacteraceae; Caulobacter;
Other
Bacteria; Proteobacteria; Alphaproteobacteria; Rhizobiales; Phyllobacteriaceae; Phyllobacterium;
Other
Bacteria; Proteobacteria; Alphaproteobacteria; Sphingomonadales; Sphingomonadaceae; Sphingomonas;
Other
Bacteria; Proteobacteria; Betaproteobacteria; Burkholderiales; Burkholderiaceae; Burkholderia;
Burkholderia ambifaria
Bacteria; Proteobacteria; Betaproteobacteria; Burkholderiales; Burkholderiaceae; Burkholderia;
Burkholderia thailandensis
Bacteria; Proteobacteria; Betaproteobacteria; Burkholderiales; Burkholderiaceae; Burkholderia;
Other
Bacteria; Proteobacteria; Betaproteobacteria; Burkholderiales; Burkholderiaceae; Other; Other
Bacteria; Proteobacteria; Betaproteobacteria; Burkholderiales; Comamonadaceae; Other; Other
Bacteria; Proteobacteria; Betaproteobacteria; Burkholderiales; Comamonadaceae; Pelomonas;
Pelomonas aquatica
Bacteria; Proteobacteria; Betaproteobacteria; Burkholderiales; Other; Other; Other
Bacteria; Proteobacteria; Betaproteobacteria; Burkholderiales; Oxalobacteraceae; Oxalobacter;
Oxalobacter formigenes
Bacteria; Proteobacteria; Betaproteobacteria; Burkholderiales; Sutterellaceae; Parasutterella;
Parasutterella excrementihominis
Bacteria; Proteobacteria; Betaproteobacteria; Burkholderiales; Sutterellaceae; Sutterella; Other
Bacteria; Proteobacteria; Betaproteobacteria; Burkholderiales; Sutterellaceae; Sutterella; Sutterella
wadsworthensis
Bacteria; Proteobacteria; Betaproteobacteria; Nitrosomonadales; Methylophilaceae; Methylophilus;
Other
Bacteria; Proteobacteria; Betaproteobacteria; Other; Other; Other; Other
Bacteria; Proteobacteria; Deltaproteobacteria; Desulfovibrionales; Desulfovibrionaceae; Bilophila;
Other
Bacteria; Proteobacteria; Gammaproteobacteria; Enterobacterales; Enterobacteriaceae; Cedecea;
Other
Bacteria; Proteobacteria; Gammaproteobacteria; Enterobacterales; Enterobacteriaceae; Citrobacter;
Other
Bacteria; Proteobacteria; Gammaproteobacteria; Enterobacterales; Enterobacteriaceae; Other; Other
Bacteria; Proteobacteria; Gammaproteobacteria; Enterobacterales; Enterobacteriaceae; Rosenbergiella;
Rosenbergiella collisarenosi
Bacteria; Proteobacteria; Gammaproteobacteria; Enterobacterales; Enterobacteriaceae; Rosenbergiella;
Rosenbergiella nectarea
Bacteria; Proteobacteria; Gammaproteobacteria; Enterobacterales; Enterobacteriaceae; Salmonella;
Salmonella enterica
Bacteria; Proteobacteria; Gammaproteobacteria; Enterobacterales; Enterobacteriaceae; Shigella;
Shigella dysenteriae
Bacteria; Proteobacteria; Gammaproteobacteria; Enterobacterales; Other; Other; Other
Bacteria; Proteobacteria; Gammaproteobacteria; Enterobacterales; Pectobacteriaceae; Pectobacterium;
Pectobacterium carotovorum
Bacteria; Proteobacteria; Gammaproteobacteria; Enterobacteriales; Enterobacteriaceae; Other; Other
Bacteria; Proteobacteria; Gammaproteobacteria; Other; Other; Other; Other
Bacteria; Proteobacteria; Gammaproteobacteria; Pasteurellales; Pasteurellaceae; Haemophilus;
Haemophilus parainfluenzae
Bacteria; Proteobacteria; Gammaproteobacteria; Pseudomonadales; Pseudomonadaceae; Pseudomonas;
Other
Bacteria; Proteobacteria; Other; Other; Other; Other; Other
Bacteria; Verrucomicrobia; Verrucomicrobiae; Verrucomicrobiales; Akkermansiaceae; Akkermansia;
Akkermansia muciniphila
Bacteria; Verrucomicrobia; Verrucomicrobiae; Verrucomicrobiales; Verrucomicrobiaceae;
Akkermansia; Other

TABLE 20
Proportion of discarded reads and counts of OTUs or ASVs after combined
clustering or denoising of amplicons with different read lengths.

| | | Percentage of discarded reads at each length | | | | | | | | | | ASVs / | |
|---|---|---|---|---|---|---|---|---|---|---|---|---|---|
| 16S | Method | 100 | 150 | 170 | 200 | 250 | 300 | 350 | 400 | 450 | O | OTUs | NIS |
| V1V3 | DADA2- | 6.43 | 8.06 | 9.26 | 10.01 | 10.57 | 11.06 | 11.59 | 12.15 | 13.40 | 13.75 | 115,446 | 18,257 |
| (forward) | DeNovo-ASV | | | | | | | | | | | | |
| | UCLUST- | 10.09 | 17.37 | 24.14 | 24.45 | 25.35 | 21.14 | 21.70 | 18.29 | 22.51 | 21.98 | 18,831 | 18,257 |
| | ClosedRef-0.97 | | | | | | | | | | | | |
| | UCLUST- | 40.13 | 56.49 | 59.89 | 63.64 | 66.54 | 68.03 | 670.3 | 71.23 | 72.96 | 72.87 | 9,359 | 18,257 |
| | ClosedRef-1.00 | | | | | | | | | | | | |
| | UCLUST- | 0 | 0 | 0 | 0 | 0 | 0 | 0 | 0 | 0 | 0 | 20,020 | 18,257 |
| | DeNovo-0.97 | | | | | | | | | | | | |
| | UCLUST- | 0 | 0 | 0 | 0 | 0 | 0 | 0 | 0 | 0 | 0 | 360,329 | 18,257 |
| | DeNovo-1.00 | | | | | | | | | | | | |
| | VSEARCH- | 0 | 0 | 0 | 0 | 0 | 0 | 0 | 0 | 0 | 0 | 10,771 | 18,257 |
| | DeNovo-0.97 | | | | | | | | | | | | |
| | VSEARCH- | 0 | 10 | 0 | 0 | 0 | 0 | 0 | 0 | 0 | 0 | 13,650 | 18,257 |
| | DeNovo-0.99 | | | | | | | | | | | | |
| | VSEARCH- | 0 | 0 | 0 | 0 | 0 | 0 | 0 | 0 | 0 | 0 | 15,668 | 18,257 |
| | DeNovo-1.00 | | | | | | | | | | | | |
| V1V3 | DADA2- | 4.53 | 5.74 | 6.13 | 6.54 | 7.25 | 8.51 | 10.34 | 10.84 | 12.58 | 13.70 | 101,887 | 18,257 |
| (reverse) | DeNovo-ASV | | | | | | | | | | | | |
| | UCLUST- | 4.99 | 5.68 | 3.95 | 3.55 | 5.76 | 5.81 | 13.39 | 11.65 | 17.72 | 21.98 | 23,810 | 18,257 |
| | ClosedRef-0.97 | | | | | | | | | | | | |
| | UCLUST- | 25.5 | 35.58 | 44.04 | 48.01 | 57.71 | 59.43 | 64.13 | 65.50 | 71.62 | 72.87 | 11,851 | 18,257 |
| | ClosedRef-1.00 | 7 | | | | | | | | | | | |
| | UCLUST- | 0 | 0 | 0 | 0 | 0 | 0 | 0 | 0 | 0 | 0 | 27,502 | 18,257 |
| | DeNovo-0.97 | | | | | | | | | | | | |
| | UCLUST- | 0 | 0 | 0 | 0 | 0 | 0 | 0 | 0 | 0 | 0 | 668,211 | 18,257 |
| | DeNovo-1.00 | | | | | | | | | | | | |
| | VSEARCH- | 0 | 10 | 0 | 0 | 0 | 0 | 0 | 0 | 0 | 0 | 9,451 | 18,257 |
| | DeNovo-0.97 | | | | | | | | | | | | |
| | VSEARCH- | 0 | 0 | 0 | 0 | 0 | 0 | 0 | 0 | 0 | 0 | 13,050 | 18,257 |
| | DeNovo-0.99 | | | | | | | | | | | | |
| | VSEARCH- | 0 | 0 | 0 | 0 | 0 | 0 | 0 | 0 | 0 | 0 | 15,710 | 18,257 |
| | DeNovo-1.00 | | | | | | | | | | | | |
| V3V5 | DADA2- | 3.56 | 5.58 | 6.00 | 6.32 | 6.78 | 7.48 | 7.88 | 8.35 | 8.75 | 9.99 | 97,800 | 18,269 |
| (forward) | DeNovo-ASV | | | | | | | | | | | | |
| | UCLUST- | 1.15 | 5.05 | 4.00 | 4.80 | 6.26 | 5.35 | 5.49 | 5.30 | 7.00 | 12.70 | 23,460 | 18,269 |
| | ClosedRef-0.97 | | | | | | | | | | | | |
| | UCLUST- | 37.11 | 35.89 | 45.40 | 46.18 | 58.54 | 59.81 | 66.87 | 63.24 | 64.86 | 74.46 | 11,354 | 18,269 |
| | ClosedRef-1.00 | | | | | | | | | | | | |
| | UCLUST- | 0 | 0 | 0 | 0 | 0 | 0 | 0 | 0 | 0 | 0 | 28,967 | 18,269 |
| | DeNovo-0.97 | | | | | | | | | | | | |
| | UCLUST- | 0 | 0 | 0 | 0 | 0 | 0 | 0 | 0 | 0 | 0 | 1,213,787 | 18,269 |
| | DeNovo-1.00 | | | | | | | | | | | | |
| | VSEARCH- | 0 | 0 | 0 | 0 | 0 | 0 | 0 | 0 | 0 | 0 | 6,510 | 18,269 |
| | DeNovo-0.97 | | | | | | | | | | | | |
| | VSEARCH- | 0 | 0 | 0 | 0 | 0 | 0 | 0 | 0 | 0 | 0 | 10,512 | 18,269 |
| | DeNovo-0.99 | | | | | | | | | | | | |
| | VSEARCH- | 0 | 0 | 0 | 0 | 0 | 0 | 0 | 0 | 0 | 0 | 14,081 | 18,269 |
| | DeNovo-1.00 | | | | | | | | | | | | |
| V3V5 | DADA2- | 3.23 | 3.95 | 4.25 | 4.52 | 5.06 | 5.90 | 6.33 | 6.94 | 8.90 | 10.02 | 85,124 | 18,269 |
| (reverse) | DeNovo-ASV | | | | | | | | | | | | |
| | UCLUST- | 1.18 | 1.60 | 1.26 | 1.52 | 1.26 | 1.94 | 2.59 | 2.92 | 13.41 | 12.70 | 23,739 | 18,269 |
| | ClosedRef-0.97 | | | | | | | | | | | | |
| | UCLUST- | 13.41 | 34.26 | 29.83 | 35.93 | 38.65 | 48.93 | 54.91 | 62.75 | 65.73 | 74.46 | 12,542 | 18,269 |
| | ClosedRef-1.00 | | | | | | | | | | | | |
| | UCLUST- | 0 | 0 | 0 | 0 | 0 | 0 | 0 | 0 | 0 | 0 | 14,924 | 18,269 |
| | DeNovo-0.97 | | | | | | | | | | | | |
| | UCLUST- | 0 | 0 | 0 | 0 | 0 | 0 | 0 | 0 | 0 | 0 | 1,010,095 | 18,269 |
| | DeNovo-1.00 | | | | | | | | | | | | |
| | VSEARCH- | 0 | 0 | 0 | 0 | 0 | 0 | 0 | 0 | 0 | 0 | 5,518 | 18,269 |
| | DeNovo-0.97 | | | | | | | | | | | | |
| | VSEARCH- | 0 | 0 | 0 | 0 | 0 | 0 | 0 | 0 | 0 | 0 | 10,041 | 18,269 |
| | DeNovo-0.99 | | | | | | | | | | | | |
| | VSEARCH- | 0 | 0 | 0 | 0 | 0 | 0 | 0 | 0 | 0 | 0 | 14,118 | 18,269 |
| | DeNovo-1.00 | | | | | | | | | | | | |
| V4 | DADA2- | 3.12 | 4.11 | 4.37 | 4.70 | NA | NA | NA | NA | NA | 5.26 | 42,922 | 19,971 |
| (forward) | DeNovo-ASV | | | | | | | | | | | | |
| | UCLUST- | 1.38 | 2.68 | 2.08 | 1.69 | NA | NA | NA | NA | NA | 3.78 | 17,463 | 19,971 |
| | ClosedRef-0.97 | | | | | | | | | | | | |
| | UCLUST- | 30.90 | 31.06 | 42.94 | 37.09 | NA | NA | NA | NA | NA | 52.47 | 11,055 | 19,971 |
| | ClosedRef-1.00 | | | | | | | | | | | | |
| | UCLUST- | 0 | 0 | 0 | 0 | NA | NA | NA | NA | NA | 0 | 7,602 | 19,971 |
| | DeNovo-0.97 | | | | | | | | | | | | |
| | UCLUST- | 0 | 0 | 0 | 0 | NA | NA | NA | NA | NA | 0 | 439,029 | 19,971 |
| | DeNovo-1.00 | | | | | | | | | | | | |
| | VSEARCH- | 0 | 0 | 0 | 0 | NA | NA | NA | NA | NA | 0 | 5,471 | 19,971 |
| | DeNovo-0.97 | | | | | | | | | | | | |
| | VSEARCH- | 0 | 0 | 0 | 0 | NA | NA | NA | NA | NA | 0 | 8,981 | 19,971 |
| | DeNovo-0.99 | | | | | | | | | | | | |
| | VSEARCH- | 0 | 0 | 0 | 0 | NA | NA | NA | NA | NA | 0 | 11,695 | 19,971 |
| | DeNovo-1.00 | | | | | | | | | | | | |
| V4 | DADA2- | 2.51 | 3.51 | 4.05 | 4.47 | NA | NA | NA | NA | NA | 5.03 | 50,898 | 19,971 |
| (reverse) | DeNovo-ASV | | | | | | | | | | | | |
| | UCLUST- | 0.65 | 2.64 | 1.26 | 1.78 | NA | NA | NA | NA | NA | 3.78 | 18,831 | 19,971 |
| | ClosedRef-0.97 | | | | | | | | | | | | |
| | UCLUST- | 15.54 | 37.01 | 42.90 | 36.59 | NA | NA | NA | NA | NA | 52.47 | 11,459 | 19,971 |
| | ClosedRef-1.00 | | | | | | | | | | | | |
| | UCLUST- | 0 | 0 | 0 | 0 | NA | NA | NA | NA | NA | 0 | 29,066 | 19,971 |
| | DeNovo-0.97 | | | | | | | | | | | | |
| | UCLUST- | 0 | 0 | 0 | 0 | NA | NA | NA | NA | NA | 0 | 1,130,881 | 19,971 |
| | DeNovo-1.00 | | | | | | | | | | | | |
| | VSEARCH- | 0 | 0 | 0 | 0 | NA | NA | NA | NA | NA | 0 | 4,883 | 19,971 |
| | DeNovo-0.97 | | | | | | | | | | | | |
| | VSEARCH- | 0 | 0 | 0 | 0 | NA | NA | NA | NA | NA | 0 | 8,753 | 19,971 |
| | DeNovo-0.99 | | | | | | | | | | | | |
| | VSEARCH- | 0 | 0 | 0 | 0 | NA | NA | NA | NA | NA | 0 | 11,806 | 19,971 |
| | DeNovo-1.00 | | | | | | | | | | | | |
| V6V9 | DADA2- | 3.95 | 5.13 | 5.96 | 6.36 | 6.68 | 7.65 | 7.93 | 18.29 | 8.74 | 10.64 | 82,525 | 14,944 |
| (forward) | DeNovo-ASV | | | | | | | | | | | | |
| | UCLUST- | 3.15 | 5.83 | 4.29 | 4.64 | 5.05 | 7.36 | 5.84 | 5.11 | 10.99 | 12.26 | 19,707 | 14,944 |
| | ClosedRef-0.97 | | | | | | | | | | | | |
| | UCLUST- | 20.41 | 33.85 | 38.87 | 40.41 | 52.04 | 57.23 | 67.96 | 62.79 | 74.13 | 84.82 | 10,100 | 14,944 |
| | ClosedRef-1.00 | | | | | | | | | | | | |
| | UCLUST- | 0 | 0 | 0 | 0 | 0 | 0 | 0 | 0 | 0 | 0 | 16,744 | 14,944 |
| | DeNovo-0.97 | | | | | | | | | | | | |
| | UCLUST- | 0 | 0 | 0 | 0 | 0 | 0 | 0 | 0 | 0 | 0 | 835,026 | 14,944 |
| | DeNovo-1.00 | | | | | | | | | | | | |
| | VSEARCH- | 0 | 0 | 0 | 0 | 0 | 0 | 0 | 0 | 0 | 0 | 5,860 | 14,944 |
| | DeNovo-0.97 | | | | | | | | | | | | |
| | VSEARCH- | 0 | | 00 | 0 | 0 | 0 | 0 | 0 | 0 | 0 | 9,191 | 14,944 |
| | DeNovo-0.99 | | | | | | | | | | | | |
| | VSEARCH- | 0 | 0 | 0 | 0 | 0 | 0 | 0 | 0 | 0 | 0 | 12,038 | 14,944 |
| | DeNovo-1.00 | | | | | | | | | | | | |
| V6V9 | DADA2- | 4.55 | 5.03 | 5.23 | 5.46 | 6.75 | 7.01 | 7.60 | 8.59 | 8.92 | 10.64 | 73,949 | 14,944 |
| (reverse) | DeNovo-ASV | | | | | | | | | | | | |
| | UCLUST- | 5.69 | 8.87 | 5.03 | 4.54 | 8.49 | 5.80 | 7.13 | 7.15 | 8.27 | 12.26 | 16,825 | 14,944 |
| | ClosedRef-0.97 | | | | | | | | | | | | |
| | UCLUST- | 28.10 | 46.15 | 57.64 | 54.26 | 62.61 | 70.05 | 73.35 | 80.74 | 75.96 | 84.82 | 8,035 | 14,944 |
| | ClosedRef-1.00 | | | | | | | | | | | | |
| | UCLUST- | 0 | 0 | 0 | 0 | 0 | 0 | 0 | 0 | 0 | 0 | 12,068 | 14,944 |
| | DeNovo-0.97 | | | | | | | | | | | | |
| | UCLUST- | 0 | 0 | 0 | 0 | 0 | 0 | 0 | 0 | 0 | 0 | 1,244,549 | 14,944 |
| | DeNovo-1.00 | | | | | | | | | | | | |
| | VSEARCH- | 0 | 0 | 0 | 0 | 0 | 0 | 0 | 0 | 0 | 0 | 5,340 | 14,944 |
| | DeNovo-0.97 | | | | | | | | | | | | |
| | VSEARCH- | 0 | 0 | 0 | 0 | 0 | 0 | 0 | 0 | 0 | 0 | 8,849 | 14,944 |
| | DeNovo-0.99 | | | | | | | | | | | | |
| | VSEARCH- | 0 | 0 | 0 | 0 | 0 | 0 | 0 | 0 | 0 | 0 | 12,008 | 14,944 |
| | DeNovo-1.00 | | | | | | | | | | | | |

Type of 16S amplicon = 16S; O = Original; Number of ASVs/OTUs = ASVs / OTUs; Number of input strains = NIS; Not Applicable = NA.

TABLE 21
Training and validation cohort information.
Data Order: Dataset ID, Adult training dataset, Internal Process
Code, Category, Geography, Clinical center, Cohort, Time Course,
Data Access, Source of metadata, Groups, Selected patient cases/
subjects, Antibiotic exposure, 16S region, Sequence type,
Sequence orientation, Platform, Publication, Journal

Meta-analysis dataset #1, FALSE, CD_Havard_V4_Paired-end, IBD, North America,
Multiple, Pediatric, Single sampling, PRJNA237362, Supplemental material of paper, “CD,
Not IBD”, 227 CD, Detailed for each subject, V4, Paired-end, Forward & reverse, Illumina,
The Treatment-Naive Microbiome in New-Onset Crohn's Disease, “Cell Host & Microbe,
2014, 15: 382-392.”
Meta-analysis dataset #2, TRUE, CD_Korean_V1V3_Single-end, IBD, South Korea, Single,
Adult, Single sampling, PRJNA240658, NCBI BioSample, “CD, HC”, “11 CD, 10 HC”, No
antibiotics within 3 months, V1-V3, Single-end, Reverse, 454, Does the intestinal microbial
community of Korean Crohn's disease patients differ from that of western patients?, “BMC
Gastroenterology, 2016, 16: 28.”
Meta-analysis dataset #3, TRUE, IBD_Havard_V3V5_Single-end, IBD, United States,
Multiple, Adult, Single sampling, PRJNA82111,
https://web.rniapps.net/iVikodak/refdash/database.php, “CD, UC, HC”, “61 CD, 47 UC, 18
HC”, Not detailed for each subject, V3-V5, Single-end, Reverse, 454, Dysfunction of the
intestinal microbiome in inflammatory bowel disease and treatment, “Genome Biology, 2012,
13: R79.”
Meta-analysis dataset #4, FALSE, IBD_Japan_V3V4_Paired-end, IBD, Japan, Unknown,
Unknown, Single sampling, PRJDB6133, NCBI BioSample, “CD, UC, HC”, “25 CD, 30 UC,
23 HC”, Unknown, V3-V4, Paired-end, Forward & reverse, Illumina, Unknown, Unknown
Meta-analysis dataset #5, TRUE, IBD_Spanish_V4_Single-end, IBD, Spain, Single, Adult,
Longitudinal sampling (follow-up), PRJNA422193, NCBI BioSample, “CD, UC, HC, HR”,
“34 CD, 33 UC, 44 HC, 39 HR”, No antibiotics within 4 weeks, V4, Single-end, Forward,
Illumina, A microbial signature for Crohn's disease, “Gut, 2017, 0: 1-10.”
Meta-analysis dataset #6, TRUE, IBD_Sweden_V4_100 bp_Single-end, IBD, Sweden, Single,
Adult, Longitudinal sampling (follow-up), PRJEB18471, NCBI BioSample, “ICD-r, ICD-nr,
CCD, UC, HC”, “49 CD, 60 UC, 9 HC”, Unknown, V4, Single-end, Forward, Illumina,
Dynamics of the human gut microbiome in inflammatory bowel disease, “Nature
Microbiology, 2017, 2: 17004.”
Meta-analysis dataset #7, TRUE, IBD_Winnipeg_V4_Paired-end, IBD, Canada, Single,
Adult, Longitudinal sampling (follow-up), PRJNA450340, NCBI BioSample, “CD, UC, HC”,
“20 CD, 19 UC, 23 HC”, Unknown, V4, Paired-end, Forward & reverse, Illumina, A
comparative study of the gut microbiota in immune-mediated inflammatory diseases-does a
common dysbiosis exist?, “Microbiome, 2018, 6: 221.”
Meta-analysis dataset #8, TRUE, IBD-IBS_Italy_V1V3_Single-end, IBD, Italy, Single, Adult,
Single sampling, PRJNA391149, NCBI BioSample, “CD, UC, HC”, “6 CD, 24 UC, 40 HC”,
No antibiotics within 3 months, V1-V3, Single-end, Reverse, 454, Fecal and Mucosal
Microbiota Profiling in Irritable Bowel Syndrome and Inflammatory Bowel Disease, “Front.
Microbiol., 2019, 10: 1655.”
Meta-analysis dataset #9, TRUE, UC_Czech_V3V4_Single-end, IBD, Czech, Single, Adult,
Single sampling, PRJNA368966, NCBI BioSample, “UC, HC”, “32 UC, 32 HC”, No
antibiotics within 3 months, V3-V4, Single-end (SRA seq is paired-end merged), Forward,
Illumina, Distinct gut microbiota profiles in patients with primary sclerosing cholangitis and
ulcerative colitis, “World J Gastroenterol 2017, 23(25): 548-4558.”
Meta-analysis dataset #10, TRUE, UC_UCSF_V4_Paired-end, IBD, United States, Single,
Adult, Single sampling, PRJNA313074, NCBI BioSample, “UC, HC”, “30 UC, 13 HC”, No
antibiotics within 2 months, V4, Paired-end, Forward & reverse, Illumina, Disease Severity
and Immune Activity Relate to Distinct interkingdom Gut Microbiome States in Ethnically
Distinct Ulcerative Colitis Patients, “mBio 2016, 7(4): e01072-16.”
Meta-analysis dataset #11, TRUE, IBD-IBS_Italy_V1V3_Single-end, IBS, Italy, Single,
Adult, Single sampling, PRJNA391149, NCBI BioSample, “IBC-C, IBS-D, IBS-M, HC”, “15
IBC-C, 14 IBS-D, 7 IBS-M, 40 HC”, Unknown, V1-V3, Single-end, Reverse, 454, Fecal and
Mucosal Microbiota Profiling in Irritable Bowel Syndrome and Inflammatory Bowel Disease,
“Front. Microbiol., 2019, 10: 1655.”
Meta-analysis dataset #12, TRUE, IBS_Italy_V3V4_Paired-end, IBS, Italy, Single, Adult,
Longitudinal sampling (intervention), PRJNA279031, NCBI BioSample, “Non-constipated
IBS, HC”, “15 Non-constipated IBS, 5 HC”, No antibiotics within 4 weeks, V3-V4, Paired-
end, Forward & reverse, Illumina, Modulation of the gut microbiota composition by rifaximin
in non-constipated irritable bowel syndrome patients: a molecular approach, “Clinical and
Experimental Gastroenterology, 2015, 8: 309-325.”
Meta-analysis dataset #13, TRUE, IBS_Korea_V3V4_Paired-end, IBS, South Korea, Single,
Adult, Single sampling, PRJNA541572, NCBI BioSample, “IBS, HC”, “30
IBS_LowCalprotectin, 19 IBS_HighCalprotectin, 43 HC”, Unknown, V3-V4, Paired-end,
Forward & reverse, Illumina, Unknown, Unknown
Meta-analysis dataset #14, TRUE, IBS_Mexico_V4_Single-end, IBS, Mexico, Single, Adult,
Longitudinal sampling (intervention), PRJNA540064, NCBI BioSample, “IBC-C, IBS-D,
IBS-M”, “16 IBC-C, 5 IBS-D, 19 IBS-M”, Unknown, V4, Single-end, Forward, Illumina,
Unknown, Unknown
Meta-analysis dataset #15, TRUE, IBS_Spanish_V4_Single-end, IBS, Spain, Single, Adult,
Longitudinal sampling (follow-up), PRJNA268708, NCBI BioSample, “IBC-C, IBS-D, IBS-
M (alternating IBS), HC”, “23 IBC-C, 47 IBS-D, 24 IBS-M, 66 HC”, No antibiotics within 3
months, V4, Single-end, Forward, Illumina, Reduction of butyrateand methane-producing
microorganisms in patients with Irritable Bowel Syndrome, “Scientific Reports, 2015,
5: 12693.”
Meta-analysis dataset #16, TRUE, IBS_UCLA_V3V5_Single-end, IBS, United States, Single,
Adult, Single sampling, PRJNA373876, NCBI BioSample, “IBS, HC”, “29 IBS, 23 HC”, No
antibiotics within 3 months, V3-V5, Single-end, Reverse, 454, Differences in gut microbial
composition correlate with regional brain volumes in irritable bowel syndrome, “Microbiome,
2017, 5: 49.”
Meta-analysis dataset #17, TRUE, IBS_Vienna_V1V2_Paired-end, IBS, Italy, Single, Adult,
Single sampling, PRJNA386442, NCBI BioSample, “IBC-C, IBS-D, IBS-M”, “5 IBC-C, 25
IBS-D, 18 IBS-M”, No antibiotics within 1 month, V1-V2, Paired-end, Forward & reverse,
Illumina, A Microbial Signature of Psychological Distress in Irritable Bowel Syndrome,
“Psychosomatic Medicine, 2018, 80: 698-709.”
Meta-analysis dataset #18, TRUE, IBS_SeattleStudy42_V4_Paired-end, IBS, United States,
Single, Adult, Single sampling, Author, Author, “IBC-C, IBS-D, IBS-M”, “25 IBC-C, 46 IBS-
D, 15 IBS-M”, No antibiotics within 2 months, V4, Paired-end, Forward & reverse, Illumina,
“Relationships of microbiome markers with extraintestinal, psychological distress and
gastrointestinal symptoms, and quality of life in women with irritable bowel syndrome”,
“Journal of Clinical Gastroenterology, 2020, 54 (2): 175-183.”
Meta-analysis dataset #19, FALSE, IBS_PHMP_V1V3_V3V5_Single-end, IBS, United
States, Single, Pediatric, Single sampling, PRJNA46339, NCBI BioSample, “IBS/FAP, HC”,
“46 IBS/FAP, 45 HC”, No antibiotics within 6 months, V1-V3, Single-end, Reverse, 454,
Gastrointestinal microbiome signatures of pediatric patients with irritable bowel syndrome,
“Gastroenterology, 2011, 141: 1782-1791.”
Meta-analysis dataset #20, FALSE, IBS_PHMP_V1V3_V3V5_Single-end, IBS, United
States, Single, Pediatric, Single sampling, PRJNA46339, NCBI BioSample, “IBS/FAP, HC”,
“46 IBS/FAP, 45 HC”, No antibiotics within 6 months, V3-V5, Single-end, Reverse, 454,
Gastrointestinal microbiome signatures of pediatric patients with irritable bowel syndrome,
“Gastroenterology, 2011, 141: 1782-1791.”
Meta-analysis dataset #21, FALSE, IBS_TCH_V4_Paired-end, IBS, United States, Single,
Pediatric, Single sampling, Author, Author, “IBS/FAP, HC”, “149 IBS/FAP, 74 HC”,
Unknown, V4, Paired-end, Forward & reverse, Illumina, Unknown, Unknown
Meta-analysis dataset #22, TRUE, CDI_UM_Schloss_V3V5_Single-end, CDI, United States,
Single, Adult, Single sampling, http://www.mothur.org/CDI_MicrobiomeModeling,
http://www.mothur.org/CDI_MicrobiomeModeling, “CDI, HC”, “94 CDI, 155 HC”, Not
detailed for each HC subject within 3 months, V3-V5, Single-end, Reverse, 454, Microbiome
Data Distinguish Patients with Clostridium difficile Infection and Non-C. difficile-Associated
Diarrhea from Healthy Controls, “mBio, 2014, 5(3): e01021-14.”
Meta-analysis dataset #23, TRUE, CDI_UFH_V1V3_Single-end, CDI, United States, Single,
Adult, Single sampling, PRJNA174594, NCBI BioSample, “CDI, HC”, “39 CDI, 40 HC”, Not
detailed for each HC subject within 3 months, V1-V3, Single-end, Reverse, 454, Intestinal
Dysbiosis and Depletion of Butyrogenic Bacteria in Clostridium difficile Infection and
Nosocomial Diarrhea, “Journal of Clinical Microbiology, 2013, 51(9): 2884-2892.”
Meta-analysis dataset #24, TRUE, CDI_Mayo2016_V4_Single-end, CDI, United States,
Single, Adult, Single sampling, PRJNA342347, NCBI BioSample, CDI, 88 CDI, Not
applicable, V4, Paired-end but only forward sequence used, Forward, Illumina, Gut
microbiome predictors of treatment response and recurrence in primary Clostridium difficile
infection, “Aliment Pharmacol Ther, 2016; 44: 715-727.”
Meta-analysis dataset #25, TRUE, CDI_Mayo2018_V4_Paired-end, CDI, United States,
Single, Adult, Single sampling, PRJNA317326, NCBI BioSample, “CDI, HC”, “85 CDI, 110
HC”, Unknown for each HC, V4, Paired-end, Forward & reverse, Illumina, Clostridioides
difficile uses amino acids associated with gut microbial dysbiosis in a subset of patients with
diarrhea, “Sci. Transl. Med., 2018, 10: eaam7019.”
Meta-analysis dataset #26, TRUE, CDI_UM_Young_V4_Paired-end, CDI, United States,
Single, Adult, Longitudinal sampling (follow-up), PRJNA307992, Supplemental material of
paper, CDI, 148 CDI samples, Not applicable, V4, Paired-end, Forward & reverse, Illumina,
Dynamics of the fecal microbiome in patients with recurrent and nonrecurrent Clostridium
difficile infection, “Genome Medicine, 2016, 8: 47.”
Meta-analysis dataset #27, TRUE, Control_UM_Young_V4_Paired-end, Control, United
States, Single, Adult, Single sampling, PRJNA386260, NCBI BioSample, HC, 178 HC, No
antibiotics within 3 months, V4, Paired-end, Forward & reverse, Illumina, Unknown,
Unknown
Validation cohort #1, FALSE, CDI_ChineseAsymCDI, CDI, China, Single, Unknown, Single
sampling, PRJNA488351, NCBI BioSample, CDI, 22 CDI, Not applicable, V3-V4, Paired-
end, Forward & reverse, Illumina, Unknown, Unknown
Validation cohort #2, FALSE, CDI_Netherlands, CDI, Netherlands, Single, Adult, Single
sampling, PRJEB30586, NCBI BioSample, CDI, 41 CDI, Not applicable, V4, Paired-end,
Forward & reverse, Illumina, “The Bacterial Gut Microbiota of Adult Patients Infected,
Colonized or Noncolonized by Clostridioides difficile”, “Microorganisms, 2020, 8, 677.”
Validation cohort #3, FALSE, CDI_Michigan, CDI, US, Single, Adult, Single sampling,
PRJNA729511, NCBI BioSample, CDI, “1, 515 CDI”, Not applicable, V4, Paired-end,
Forward & reverse, Illumina, Unknown, Unknown
Validation cohort #4, FALSE, IBD_Japan_V3V4_Paired-end, IBD, Japan, Unknown,
Unknown, Single sampling, PRJDB6133, NCBI BioSample, “CD, UC, HC”, “25 CD, 30 UC,
23 HC”, Unknown, V3-V4, Paired-end, Forward & reverse, Illumina, Unknown, Unknown
Validation cohort #5, FALSE, IBD_UCC_Canada, IBD, Ireland, Unknown, Adult,
Longitudinal sampling (follow-up), PRJNA414072, Author, “CD, UC, HC”, “186 CD, 144
UC, 79 HC”, Not detailed for each subject, V3-V4 (only use V4 forward seq), Paired-end,
Forward & reverse, Illumina, Ranking microbiome variance in inflammatory bowel disease: a
large longitudinal intercontinental study, “Gut, 2020, 0: 1-12”
Validation cohort #6, FALSE, IBD_UCC_Canada, IBD, Canada, Unknown, Adult,
Longitudinal sampling (follow-up), PRJNA414072, Author, “CD, UC, HC”, “117 CD, 84 UC,
82 HC”, Not detailed for each subject, V3-V4 (only use V4 forward seq), Paired-end, Forward
& reverse, Illumina, Ranking microbiome variance in inflammatory bowel disease: a large
longitudinal intercontinental study, “Gut, 2020, 0: 1-12”
Validation cohort #7, FALSE, AGP-IBD, IBD, US, Not applicable, Adult (main), Single
sampling, PRJEB11419,
https://figshare.com/articles/dataset/Full_American_Gut_Project_mapping_file/6137315,
IBD, 162 IBD (self-reported), No antibiotics within 6 months (majority), V4, Single-end,
Forward, Illumina, American Gut: an Open Platform for Citizen Science Microbiome
Research, mSystems 3: e00031-18
Validation cohort #8, FALSE, IBS_LowFODMAP, IBS, New Zealand, Single, Adult,
Longitudinal sampling (follow-up), PRJNA392762, NCBI BioSample, IBS, 54 IBS samples
(control diet), Unknown, V4, Paired-end, Forward & reverse, Illumina, Long-term irritable
bowel syndrome symptom control with reintroduction of selected FODMAPs, “World J
Gastroenterol, 2017, 23(25): 4632-4643.”
Validation cohort #9, FALSE, IBS_Japan, IBS, Japan, Single, Adult, Single sampling,
PRJNA637763, NCBI SRA, IBS, 85 IBS, No antibiotics within 1 months, V1-V2, Paired-end,
Forward & reverse, Illumina, Usefulness of Machine Learning-Based Gut Microbiome
Analysis for Identifying Patients with Irritable Bowels Syndrome, “J. Clin. Med. 2020, 9(8):
2403.”
Validation cohort #10, FALSE, IBS_Seattle-Study93, IBS, US, Single, Adult, Single
sampling, Author, Author, IBS, 71 IBS, Unknown, V4, Paired-end, Forward & reverse,
Illumina, Unknown, Unknown
Validation cohort #11, FALSE, IBS_Yoga, IBS, Germany, Single, Adult, Longitudinal
sampling (follow-up), PRJEB24421, NCBI BioSample, IBS, 88 IBS samples (yoga group),
Unknown, V3-V4, Paired-end, Forward & reverse, Illumina, Randomised clinical trial: yoga
vs a low-FODMAP diet in patients with irritable bowel syndrome, “Aliment Pharmacol Ther,
2018, 47: 203-211.”
Validation cohort #12, FALSE, AGP-IBS, IBS, US, Not applicable, Adult (main), Single
sampling, PRJEB 11419,
https://figshare.com/articles/dataset/Full_American_Gut_Project_mapping_file/6137315, IBS,
“1, 161 IBS (self-reported)”, No antibiotics within 6 months (majority), V4, Single-end,
Forward, Illumina, American Gut: an Open Platform for Citizen Science Microbiome
Research, mSystems 3: e00031-18
Validation cohort #13, FALSE, LifeLines-IBS, IBS, Netherlands, Not applicable, Adult
(main), Single sampling, EGAD00001003453, https://www.lifelines.nl/ (Need Purchase for
data access), IBS, 115 IBS (self-reported), Not clear at the time of stool sampling, V4, Paired-
end, Forward & reverse, Illumina, Many, Many

TABLE 22
Simulation of 16S rDNA amplicon sequences from NCBI 16S rRNA RefSeq database.
16S Region = 16S; Amplicon size of E. coli 16S template = Amp; Size selection of
amplicons = SSA; Count of sequences (strains) of NCBI 16S rRNA RefSeq = Count; Before
simulation = Before; After simulation = After.

| | | | | SSA | | Count | |
|---|---|---|---|---|---|---|---|
| 16S | Forward (5′→3′) | Reverse (5′→3′) | Amp | min. | max. | Before | After |
| V1-V3 | AGAGTTTGAT | CCAGCAGCCGC | 490 | 420 | 510 | 20,160 | 90% |
| | CATGGCTCAG | GGTAAT | | | | | input |
| | (SEQ ID NO: 1) | (SEQ ID NO: 2) | | | | | |
| V3-V5 | CCTACGGGAG | ACTCAAATGAA | 551 | 525 | 561 | 20,160 | 90% |
| | GCAGCAG | TTGACGG | | | | | input |
| | (SEQ ID NO: 3) | (SEQ ID NO: 4) | | | | | |
| V4 | CCAGCAGCCG | ATTAGATACCC | 253 | 248 | 258 | 20,160 | 99% |
| | CGGTAA | TGGTAGTCC | | | | | input |
| | (SEQ ID NO: 5) | (SEQ ID NO: 6) | | | | | |
| V6 | AACGCGAAGA | AAGTCGTAACA | 507 | 477 | 527 | 20,160 | 75% |
| V9 | ACCTTAC | AGGTAACCGTA | | | | | input |
| | (SEQ ID NO: 7) | (SEQ ID NO: 8) | | | | | |

### Example 3—Determining Optimal Sequence Lengths for Accurate Taxonomic Profiling of 16S Amplicons

Most 16S pipelines trim amplicon reads to equal short sequence lengths after quality control procedures resulting in potential compositional and taxonomic bias (FIG. 1A). Submitting short and long 16S reads for downstream processing can potentially reduce this bias but its analytics represents a bioinformatics challenge. Notably, optimal amplicon length for sequence clustering/denoising and taxonomic resolution needs to be established for each microbial species investigated. To assess the feasibility of such a bioinformatics approach, the inventors simulated 16S amplicon data of variable length and maintained an identical allocated sequence count (randomly assigned from 1 to 50) for benchmarking the accuracy of different sequence clustering/denoising tools. For commonly used 16S variable regions (V1-V3, V3-V5, V4 and V6-V9), closed-reference analysis using UCLUST discarded large proportions of amplicon reads even when a comprehensive reference database (SILVA release 132) was used, and results were strongly biased towards higher sequence identity and longer reads (Table 20). Although the DADA2 pipeline retained more reads in this simulated analysis, it still discarded >2% of sequences, with singleton reads being disproportionally excluded. By contrast, de novo clustering tools retained all sequence reads setting precedent for accurate compositional profiling (Table 20). By performing pairwise Spearman correlations between any two variable lengths (as two independent samples) in OTU/ASV output tables, the inventors found that applying 99% similarity for clustering amplicons in VSEARCH conferred the highest correlation coefficients across wider length ranges in all 16S variable regions tested (data available upon request). Spearman coefficients increased progressively with longer reads, enabling establishment of minimum amplicon length thresholds (Spearman's rho >0.75) and optimal amplicon length ranges for sequence clustering of variable length input data (FIG. 1B and FIGS. 7A-7C).

The above selected amplicon sequence ranges were then applied to assess whether qualified variable read lengths generated from different 16S regions provided accurate taxonomic annotation. Using random and repeat sequences previously reported for benchmarking of taxonomic over-classification by Murali et al. (ref. 10), the inventors found that default settings in BLCA11 did not annotate these sequences whereas other popular taxonomic classifiers, including RDP classifier and SINTAX generated high false positive hits (ref. 10). Because random and repeat sequences do not accurately reflect uncharacterized/unidentified species that could contribute to taxonomical over-classification in a microbiome community, the inventors used simulated amplicon data of unannotated 16S sequences (down to family-rank from the RDP database 11.5) to determine optimal settings in BLCA. The inventors found that taxonomic over-classification is highly dependent on 16S variable region, identity, and coverage of sequence alignment in BLCA (FIG. 8). Over-classification rates were reduced to below 5% (V1-V3, V3-V5, V6-V9) and 10% (V4) when we increased the threshold of sequence identity and coverage to 0.99, without applying bootstrap confidence thresholds for taxonomic selection (FIG. 8). Thus, sequence identity and coverage thresholds of 0.99 were applied in BLCA to conduct the following benchmarking.

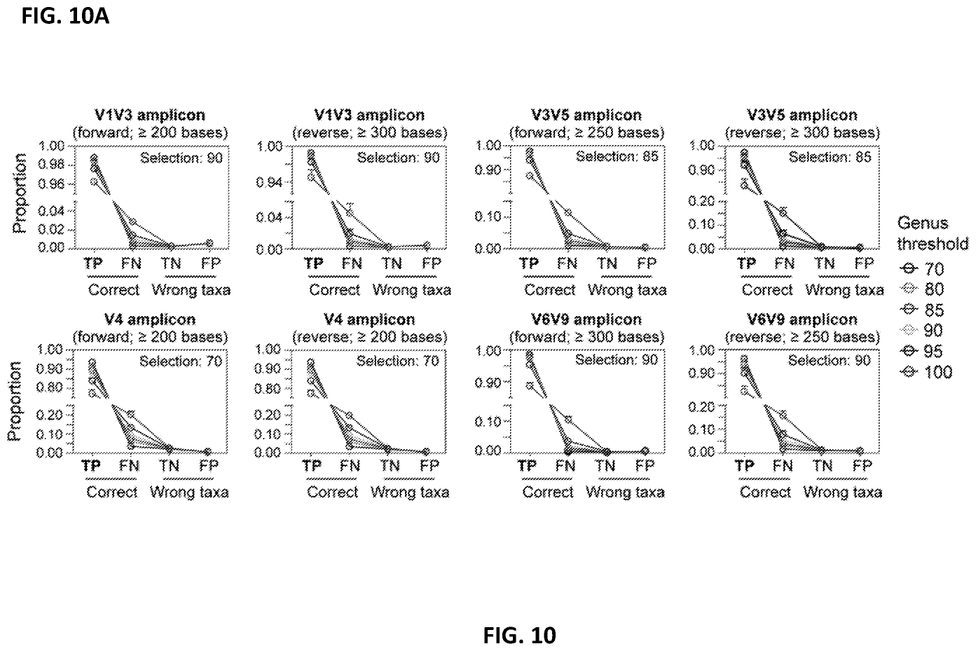

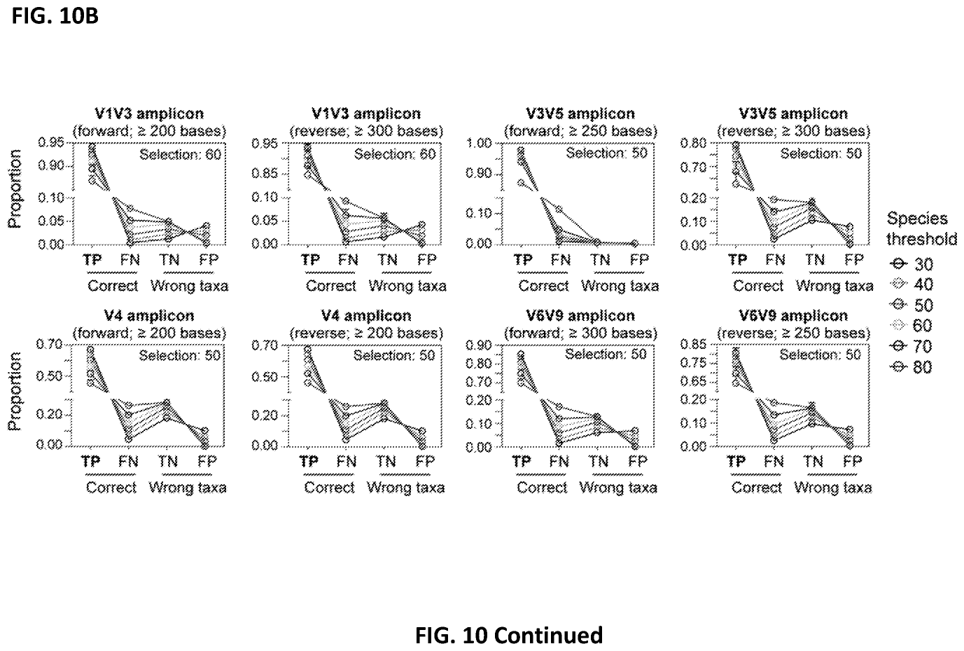

The inventors utilized the above threshold settings in BLCA to annotate simulated amplicons of variable length generated from known taxonomic lineages in the NCBI 16S RefSeq database. To calculate taxonomic accuracy, the inventors compared BLCA annotations against input lineage 16S data (ground truth). The inventors then calculated optimal confidence scores and proportions of correctly assigned taxonomic annotations for each qualified sequence length and found that correctly assigned amplicons were significantly increased towards longer read length (FIGS. 1C-1D and FIGS. 9A-9D). Surprisingly, the inventors also observed that confidence scores for incorrect annotations were significantly increased with longer read length, and that misclassification rates were highly dependent on 16S sequence orientation and variable region analyzed (FIGS. 9A-9D). This finding relates to the observation that increasing amplicon length generally improves taxonomic accuracy at species-rank relative to genus-rank, as the latter has more capacity for degeneracy. Because the inventors showed that it is not appropriate to apply universal confidence thresholds to all types of 16S amplicon data, they calculated optimal region-specific confidence thresholds in order to achieve accurate taxonomic annotation for all common types of amplicon data that could be used in a meta-analyses (FIGS. 10A-10B). This conceptual approach formed the basis of a 16S meta-analysis using a new taxonomic binning strategy.

### Example 4—Taxa4Meta: A ‘Best Practices’ Taxonomic Profiler for 16S Meta-Analysis

Based on the above described benchmarking results of simulated 16S amplicon data, the inventors designed the bioinformatics pipeline “Taxa4Meta” for accurate taxonomic profiling of 16S rDNA amplicon data generated from different sequencing strategies (FIG. 2A). Taxa4Meta was constructed to maximize the use of clinically archived 16S datasets by adopting a variable sequence length analysis strategy that can be applied to multiple amplicon regions. To obtain accurate taxonomic profiles, we introduced two critical workflow-specific settings: (1) VSEARCH-based de novo sequence clustering with 99% similarity was applied to 16S amplicon data with optimal sequence length range as determined above for each amplicon data type; (2) confident species calls were achieved by using BLCA with stringent sequence alignment (99% identity and 99% coverage), and by applying region-specific confidence scores as determined above. OTUs that were not annotated by BLCA were fed into and classified by the IDTAXA program using its pre-built RDP training set (version 16; curated by program developer). As a last step, Taxa4Meta OTU tables were generated by collapsing taxonomical features down to species-rank without processing for random rarefaction that could result in a biased taxonomic profile.

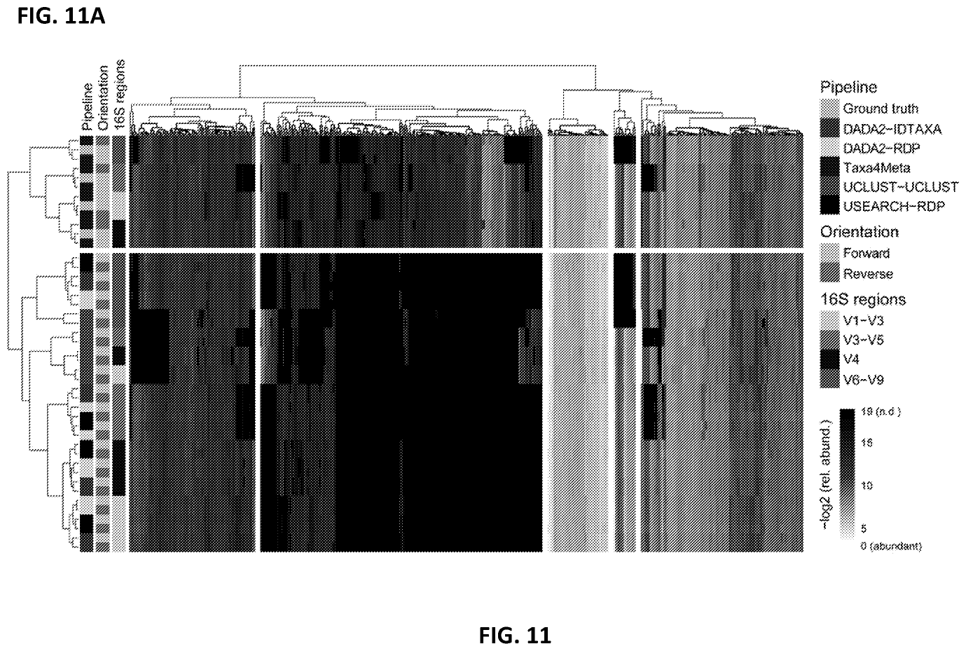

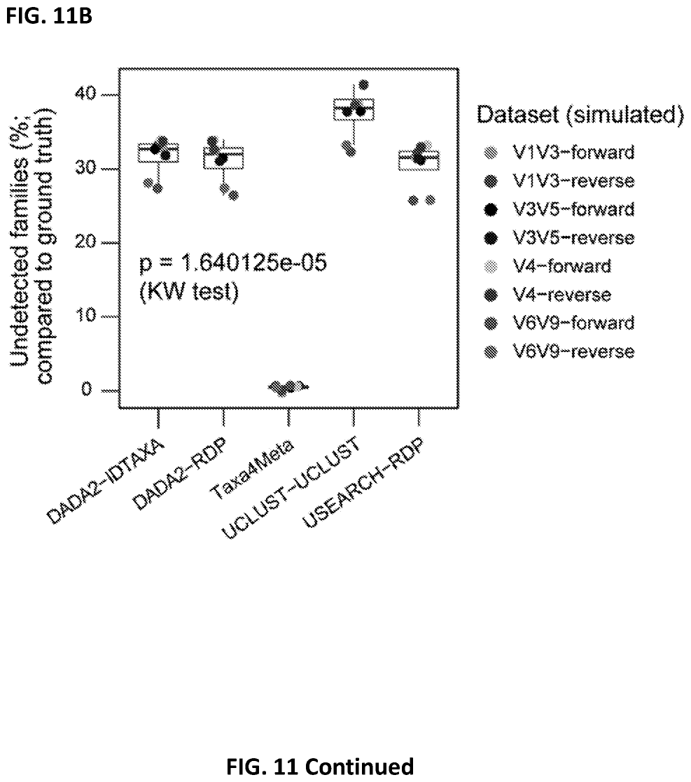

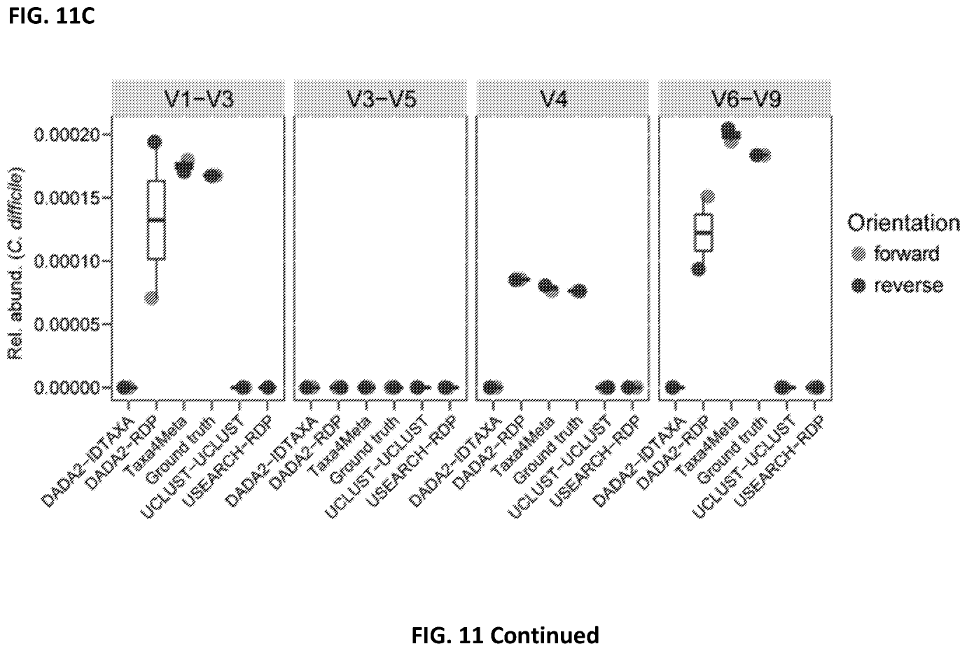

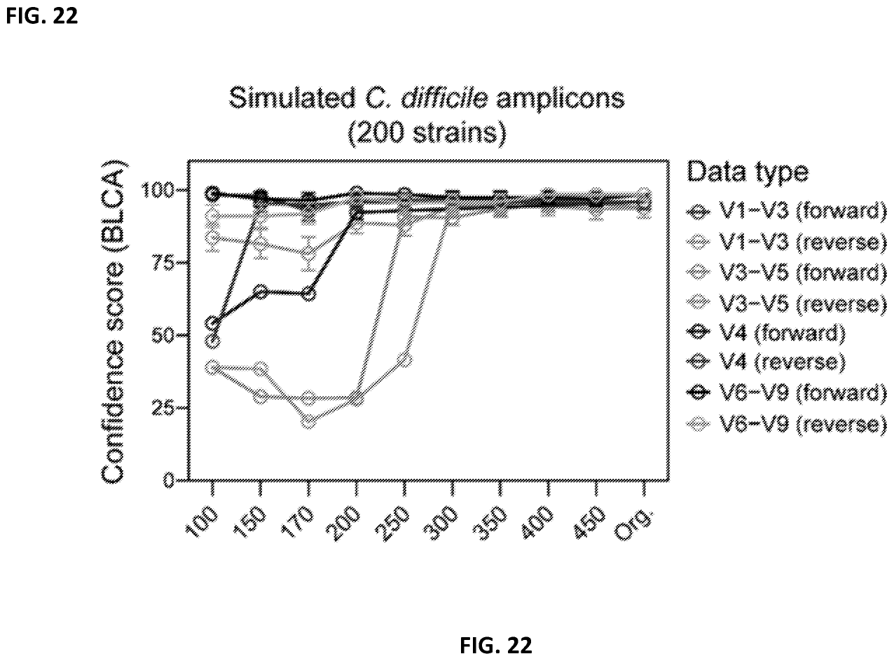

To test the taxonomic profiling accuracy of Taxa4Meta, the inventors generated complex mock communities with defined and cultivable bacteria as benchmarking input. First, variable length amplicons from diverse 16S sequences representing the NCBI 16S RefSeq database (>20.000 bacterial strains representing >14,000 species from >2.900 genera) were simulated. For benchmarking Taxa4Meta, amplicon length ranges that provide optimal taxonomic annotation for each distinct 16S variable region (FIG. 1B and FIGS. 7A-7C) were selected. Taxa4Meta performance was then critically compared against four state-of-the art 16S pipelines and the input data (ground truth). Because commonly used 16S pipelines rely on different reference databases for taxonomic annotation, taxonomic profiles were interpreted at family rank as this is more consistently represented across databases. Simulated datasets containing defined sequence abundances and taxonomic lineages (ground truth) were used to generate Spearman correlations to compare qualified input data with compositional profiles generated by individual 16S pipelines. Side-by-side comparisons of Taxa4Meta against DADA2. UCLUST and USEARCH pipelines demonstrated the former outperformed the other taxonomic profilers as it generated significantly higher Spearman correlation coefficients across all 16S regions tested (FIG. 2B). Using an independent method of hierarchical clustering, it was further demonstrated that only Taxa4Meta profiles clustered with ground truth input profiles (FIG. 11A), whereas the other taxonomic profilers failed to detect a significant number of families across the four 16S regions tested, i.e., >30% of families were not detected compared with 0.4% omitted by Taxa4Meta (FIG. 11B). The results highlight the utility of Taxa4Meta in generating accurate taxonomic profiles of complex microbiome communities, and this is evident down to species-rank as demonstrated for the detection of C. difficile, a pathogen required for the clinical diagnosis of CDI (FIG. 11C and FIG. 22).

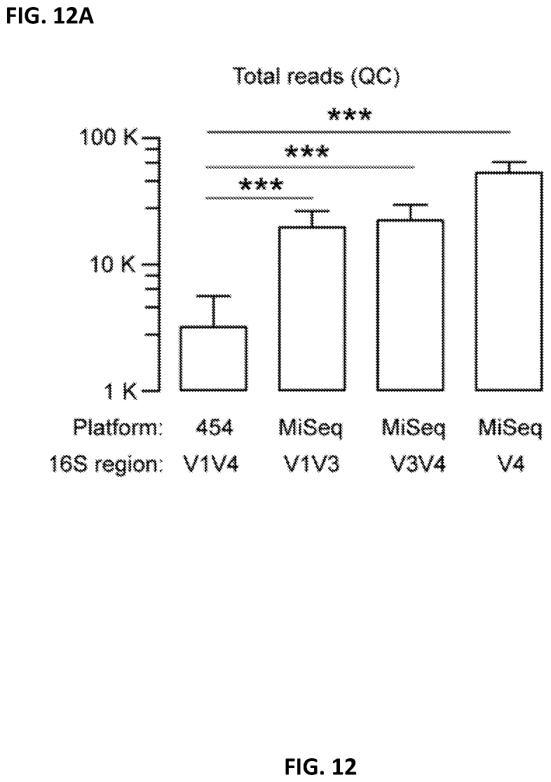

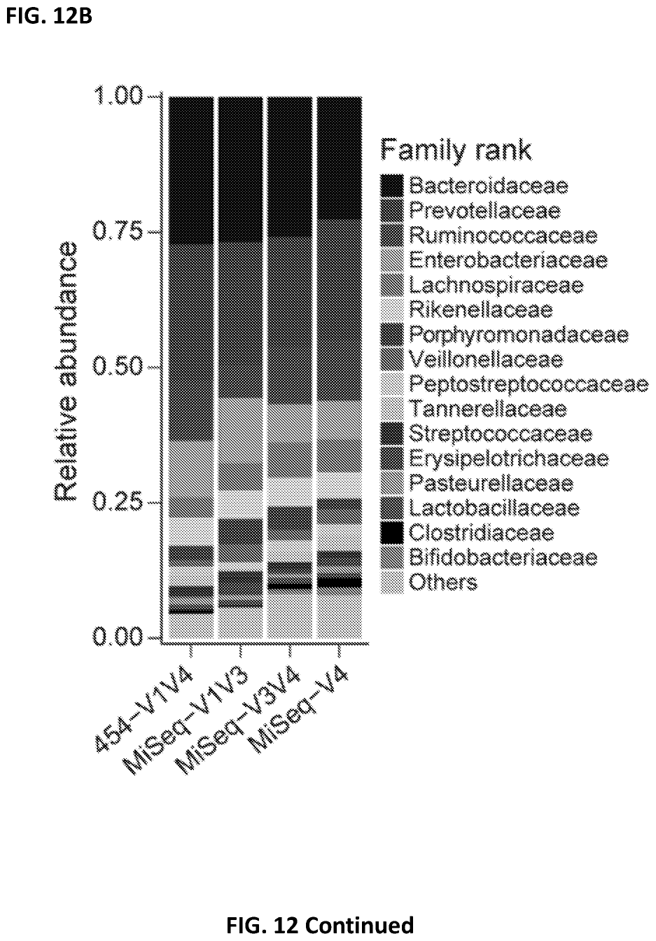

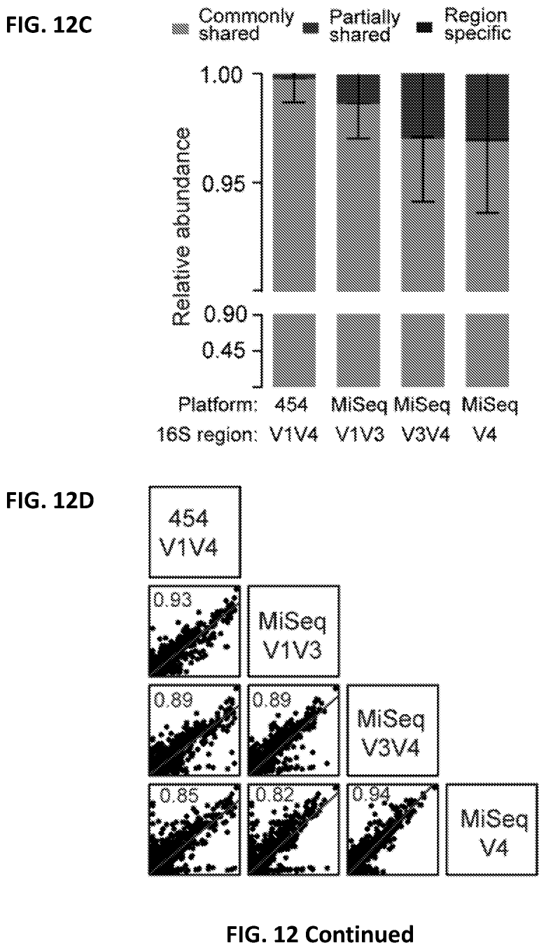

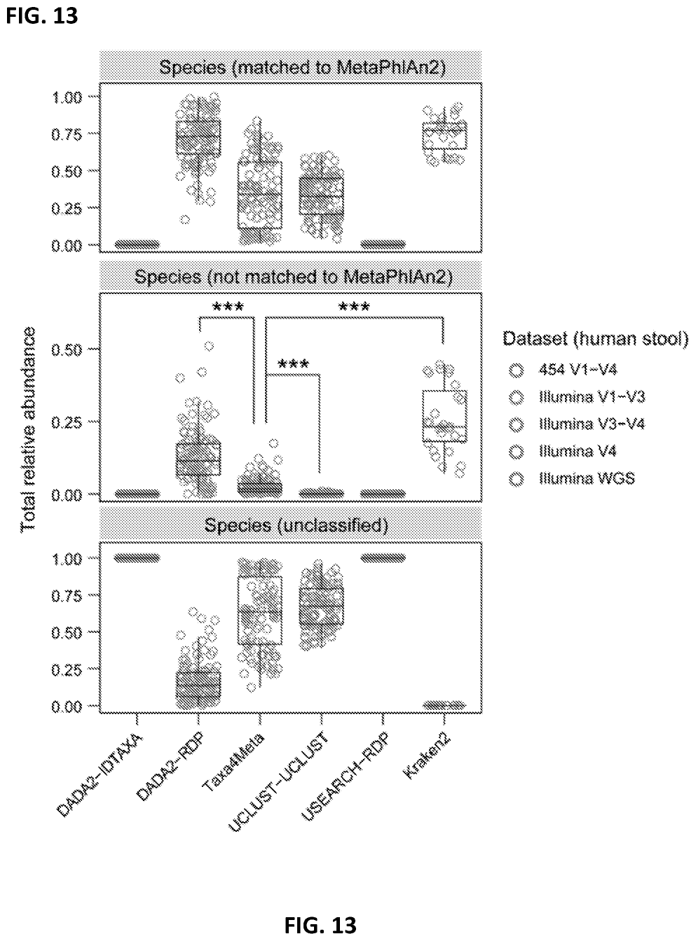

To investigate how Taxa4Meta performed with real-world microbiome datasets, the inventors benchmarked different 16S pipelines using a South Korean cohort of healthy subjects where individual fecal DNA extract underwent comprehensive 16S profiling and shotgun metagenomic sequencing¹². As the inventors observed using complex simulated microbiome communities. Taxa4Meta family-rank profiles clustered together with Kraken2-generated annotations which are regarded as a gold standard reference method because of its high family-rank taxonomic accuracy using metagenomic data¹³ (FIG. 2C). By adopting an independent method of pairwise abundance-weighted Jaccard distance calculations. Taxa4Meta profiles further demonstrated the best close distance to Kraken2 profile (FIG. 2D), and this was consistently observed across all 16S data types investigated regardless of sequencing depth (FIGS. 12A-12D). The inventors also evaluated the accuracy of Taxa4Meta species-rank profiles compared with MetaPhlAn2-generated taxonomy as this has higher precision than Kraken2 in avoiding species misclassification¹⁴. Taxa4Meta stringently controls for species misclassification (FIG. 13). Collectively, the findings show that collapsed taxonomic profiles generated by Taxa4Meta are highly accurate and suitable for 16S meta-analysis of amplicon data generated from diverse sequencing strategies.

### Example 5—Population-Scale Meta-Analysis to Define the Healthy Human Gut Microbiome

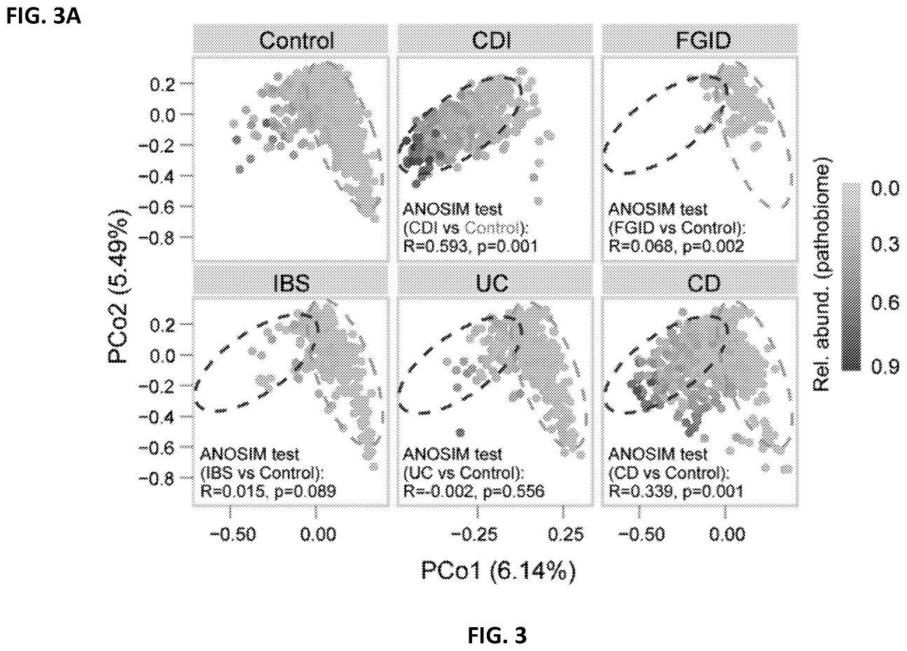

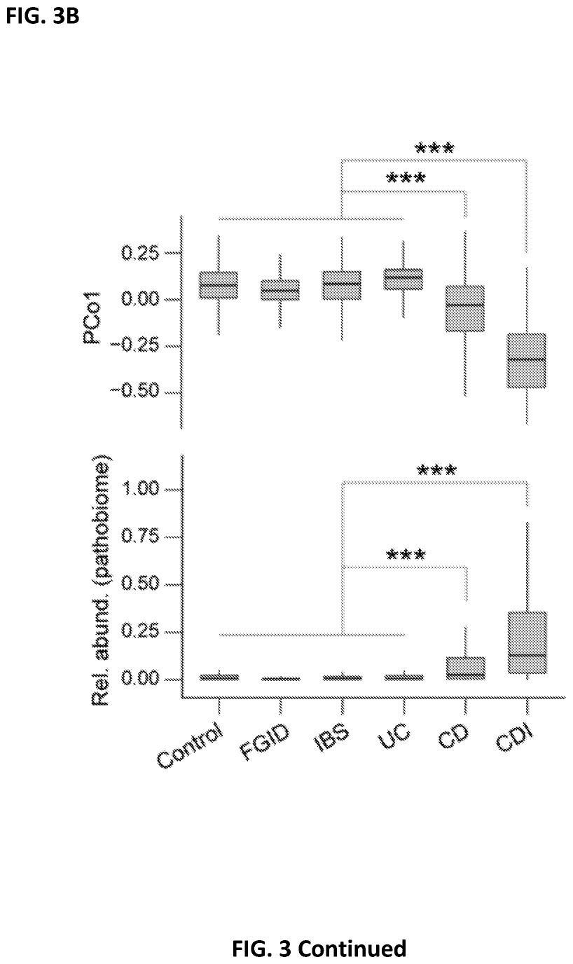

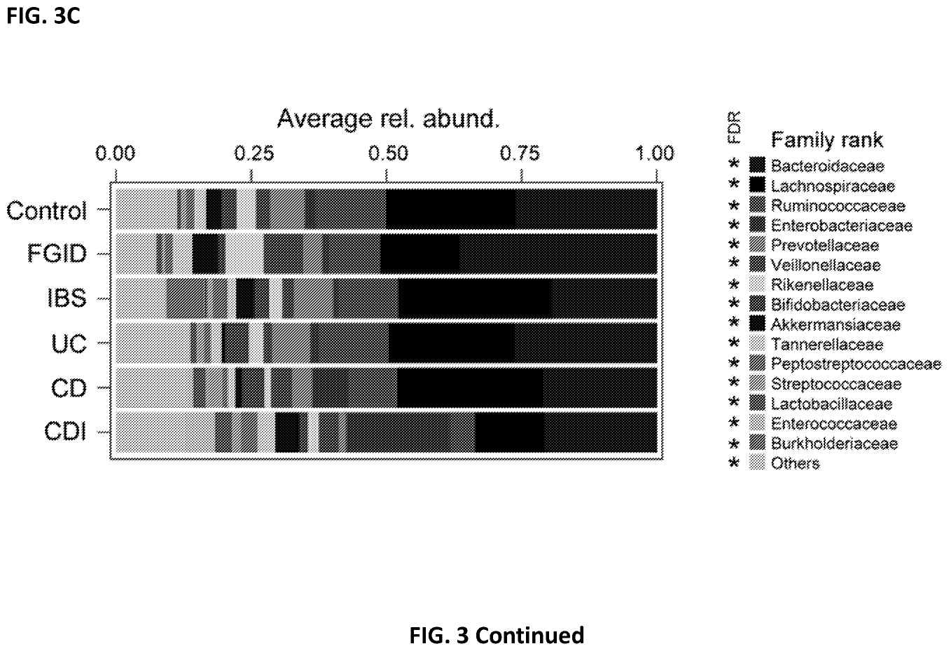

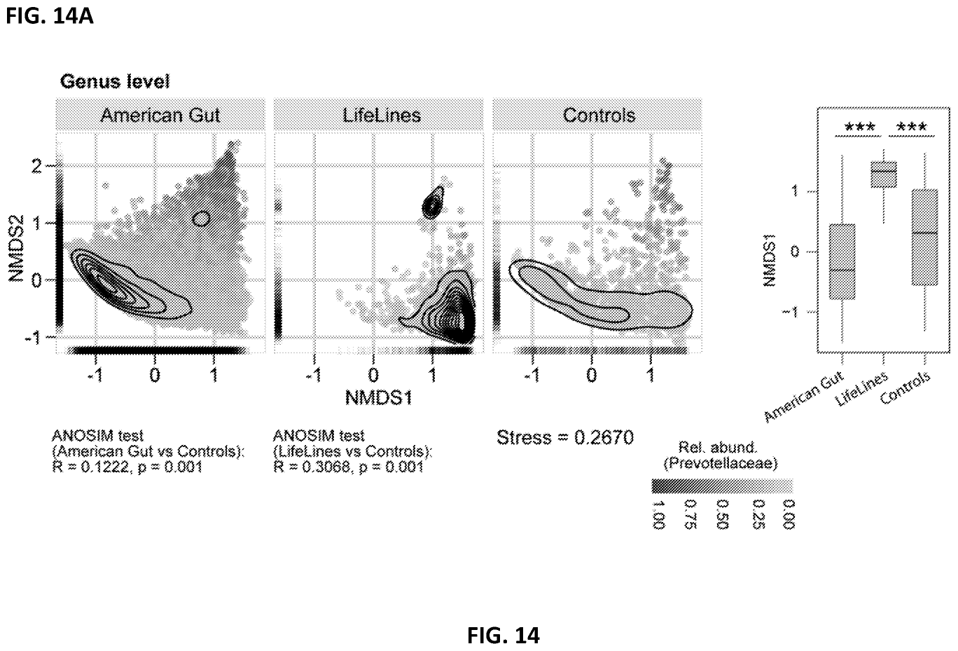

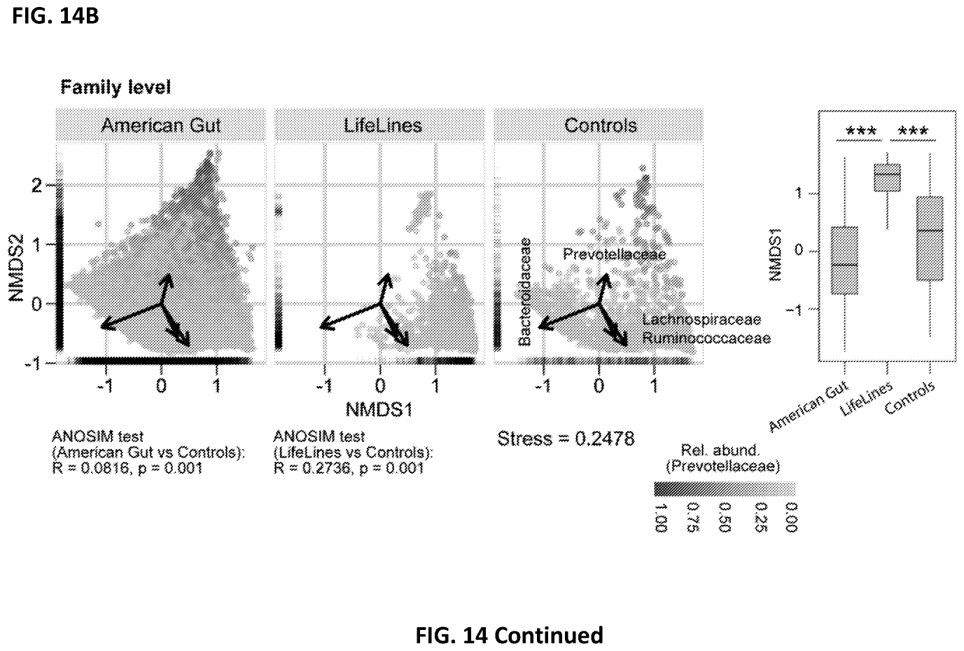

Defining the healthy human gut microbiome remained a major challenge because it is influenced by many individual factors, including age, genetics, diet, environment, lifestyle and transmission². In addition to these influences, discordant analytical methods and small cohort sizes are important determinants in how to reliably chose to characterize the healthy human microbiome. The inventors applied the Taxa4Meta pipeline to perform a meta-analysis of diverse 16S regions and sequencing platforms to identify common microbiome features in over 900 subjects with no documented gastrointestinal disease across North America. Europe. Asia and Australasia (Table 21). The inventors further compared taxonomic profiles of control subjects with over 13.000 participants in the American Gut Project¹⁵ and LifeLines cohorts¹⁶. Using Bray-Curtis dissimilarity distance based β-diversity analysis, it was shown that control subjects sequenced across diverse technology platforms shared a similar sample distribution or microbiome variation pattern with American Gut at both genus- and family-rank abundance profiles (FIGS. 14A-14B). Because the inventors identified a significant enterotype bias when comparing American Gut or meta-analysis controls with the European LifeLines cohort, they designed the meta-analysis to include study controls that spanned all the major classical gut enterotypes in order to facilitate accurate downstream disease classification at a population-scale level. Non-Prevotella enterotypes dominated by Bacteroidaceae, Lachnospiracede and Ruminicoccacede represented the healthy gut microbiome in controls from the 16S meta-analysis cohorts (FIGS. 14A-14B). The inventors further identified some outlier controls that were dominated by high abundance of pathobiome, defined as the presence of Enterococcus. Streptococcus, Clostridioides, Escherichia/Shigella, Salmonella, Klebsiella and Pseudomonas (FIGS. 3A-3B). Given that Prevotella and pathobiome-dominated gut microbiota are associated with chronic inflammatory conditions^{5,17,18}, the results highlight the need for population-scale analyses to consider enterotypes and pathobiome when defining the healthy human microbiome, especially in the context of dysbiosis-associated gastrointestinal disease.

### Example 6—Dysbiosis in Chronic Human Diarrheal Disease

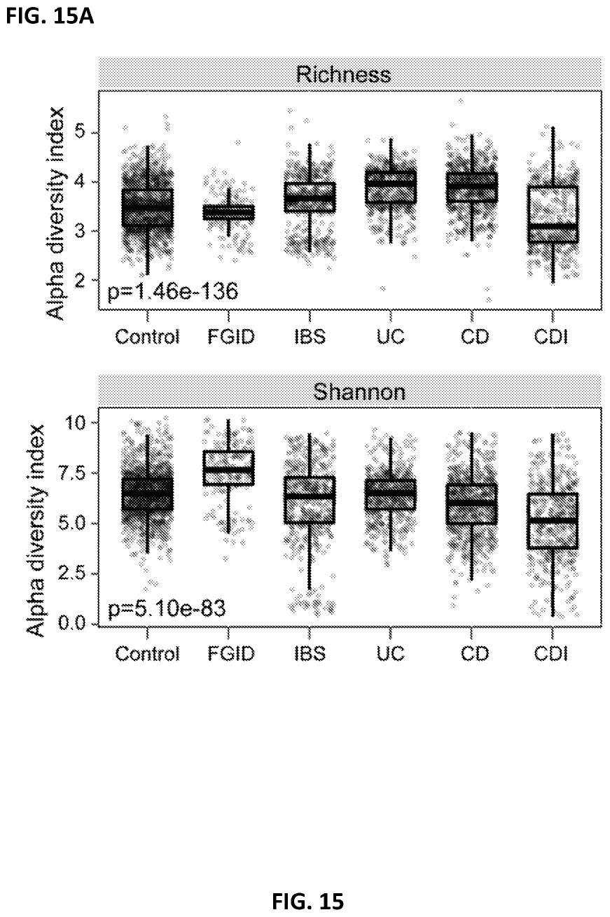

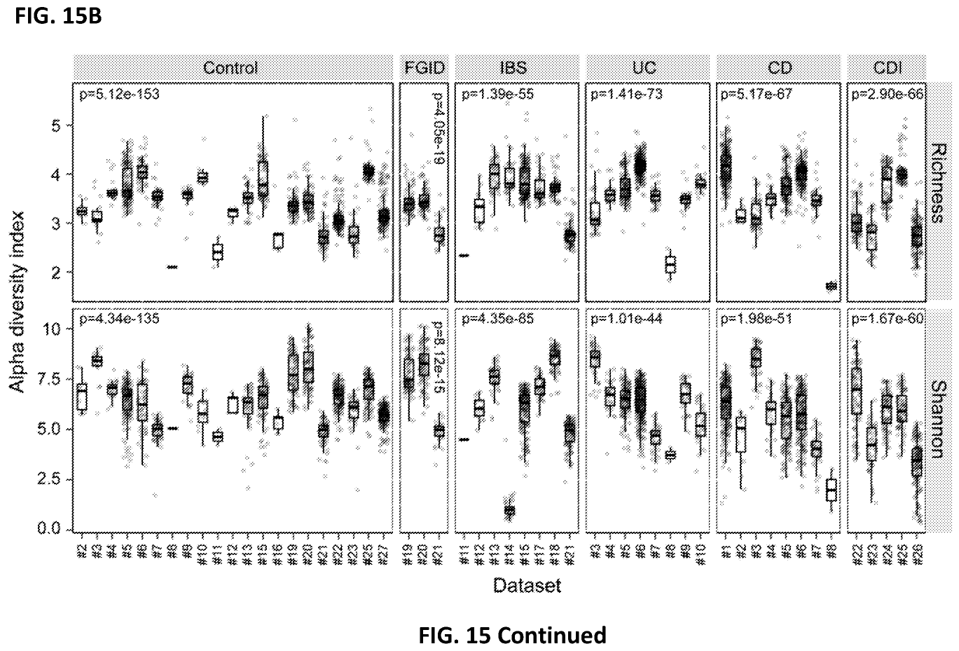

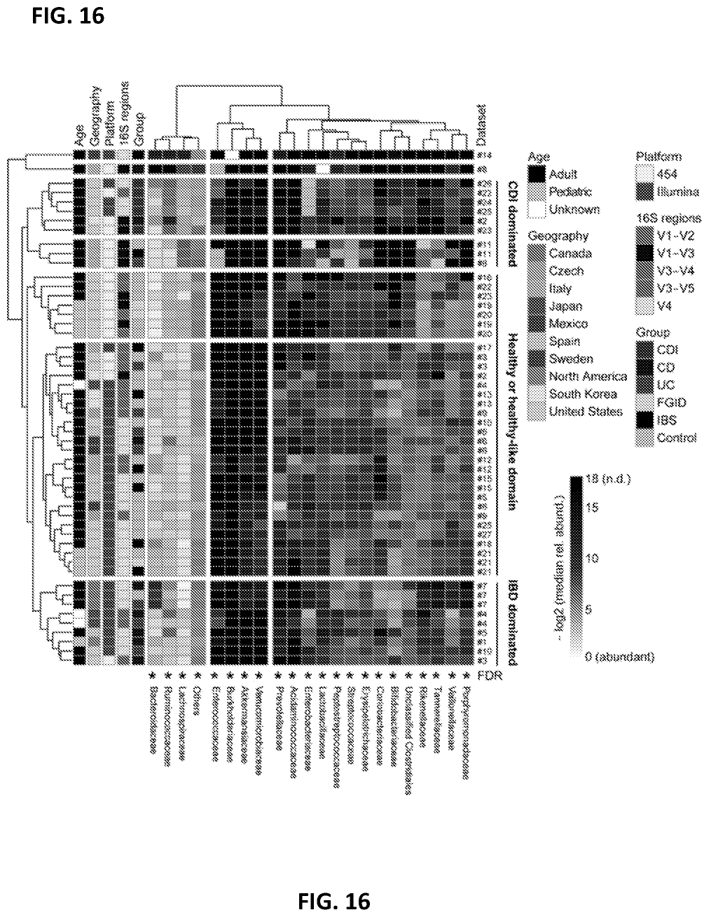

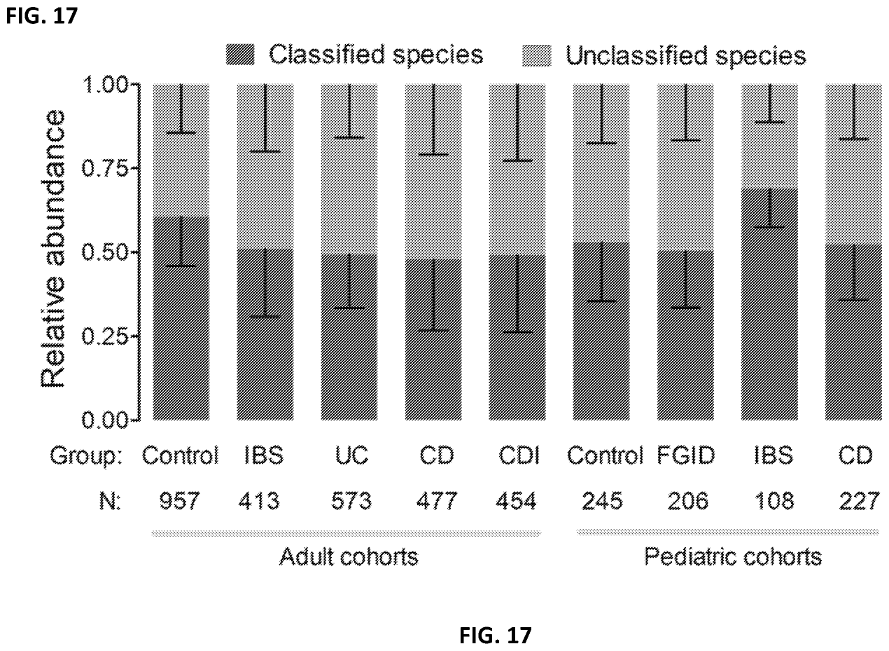

Using the Taxa4Meta pipeline, the inventors analyzed fecal microbiome data sequenced over multiple 16S regions on Illumina and 454 pyrosequencing platforms, analyzing over 5,500 matched controls and clinically confirmed diarrheal patients with CDI, IBD, IBS, and non-IBS functional gastrointestinal disorders (FGID) from North America. Europe. Asia and Australasia. α-Diversity indices calculated from Taxa4Meta OTU tables were significantly lower in CDI cases compared with controls or other diarrheal diseases (FIG. 15A), but this was an inconsistent feature among clinical cohorts sequenced across different 16S regions (FIG. 15B). In agreement with prior studies^{5,18}, the abundance of Enterobacteriaceae and Enterococcacede were significantly elevated in CDI and CD patients compared with matched controls or IBS and UC cases (FIG. 3C and FIG. 16). Collapsed species profiles generated by Taxa4Meta contained classified and unclassified members representing 54% and 46% of total abundance, respectively (FIG. 17), allowing confident assignment of species calls for further data mining. Abundance-weighted Jaccard distance based β-diversity analysis demonstrated a healthy-like microbiome community structure in IBS and UC patients, whereas significant dysbiosis was consistently detected in CDI and CD cases (FIGS. 3A-3B). This observation is in agreement with prior case-control matched studies¹⁹ that reported subtle microbiome differences in IBS and FGID patients when compared to healthy controls (FIG. 16). By contrast, the inventors found that CDI and CD patients were readily distinguished from other diarrheal diseases by the abundance of pathobiome (FIGS. 3A-3B), a reflection of gut dysbiosis favoring engraftment and expansion of potential pathogens. Pathobiome-dominated enterotypes found in CD and CDI patients are primarily composed of Enterobacteriaceae and Enterococcaccae, which were shown to occur independently of 16S region, sequencing platforms, age or geography (FIG. 16). Thus, calculation of specific β-diversity distance metrics and pathobiome abundance become useful tools in defining the core microbiome features of specific diarrheal disease types.

Using hierarchical clustering analysis for family abundance profiles the inventors demonstrated that 4 out of 8 UC cohorts were clustered together with control and IBS patients, whereas the remaining UC cohorts and the majority of CD cohorts formed a unique IBD-specific cluster (FIG. 16). This finding was not recapitulated by the microbiome meta-analysis conducted by Duvallet et al.²⁰ because UC and CD patients were grouped together for microbiome comparisons against controls. A recent systematic literature review highlighted the significant decrease of Faecalibacterium prausnitzii, an anti-inflammatory gut commensal in both UC and CD patients⁵. The inventors confirmed this finding in their meta-analysis of CD and UC cases (Tables 9-17), but failed to demonstrate significant alterations in Eubacterium rectale and Escherichia abundance in UC patients as previously reported⁵, which could reflect microbiome variations seen in UC cohorts as demonstrated in the meta-analysis. Several novel top ranked disease-associated species were identified including Fusicatenibacter saccharivorans (Control-specific), and Bacteroides xylanisolvens and Romboutsia timonensis (less prevalent in IBD) which have not been reported in previous studies. Other unique findings included decreases in Anaerostipes hadrus and Eubacterium rectale in CDI patients only (Tables 9-17), these features were exploited develop disease-specific classifiers.

### Example 7—Pan-Microbiome Profiling Outperforms Individual 16S Region-Specific or Platform-Specific Analysis for Disease Classification

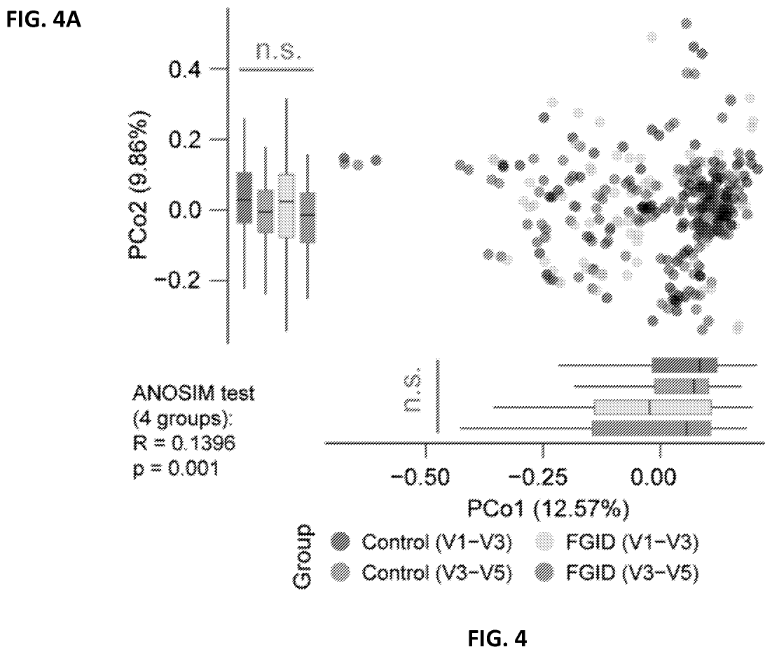

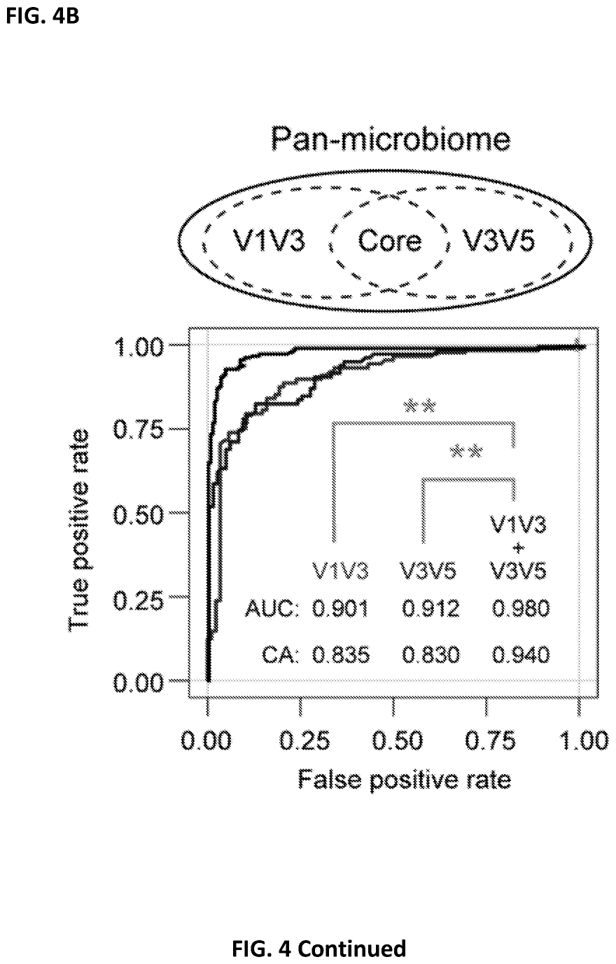

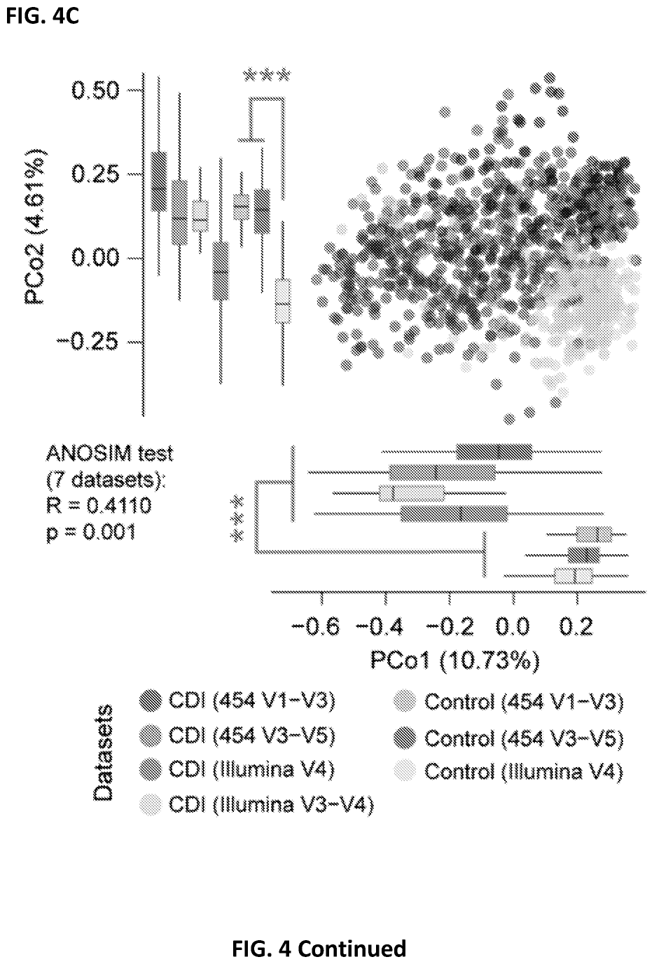

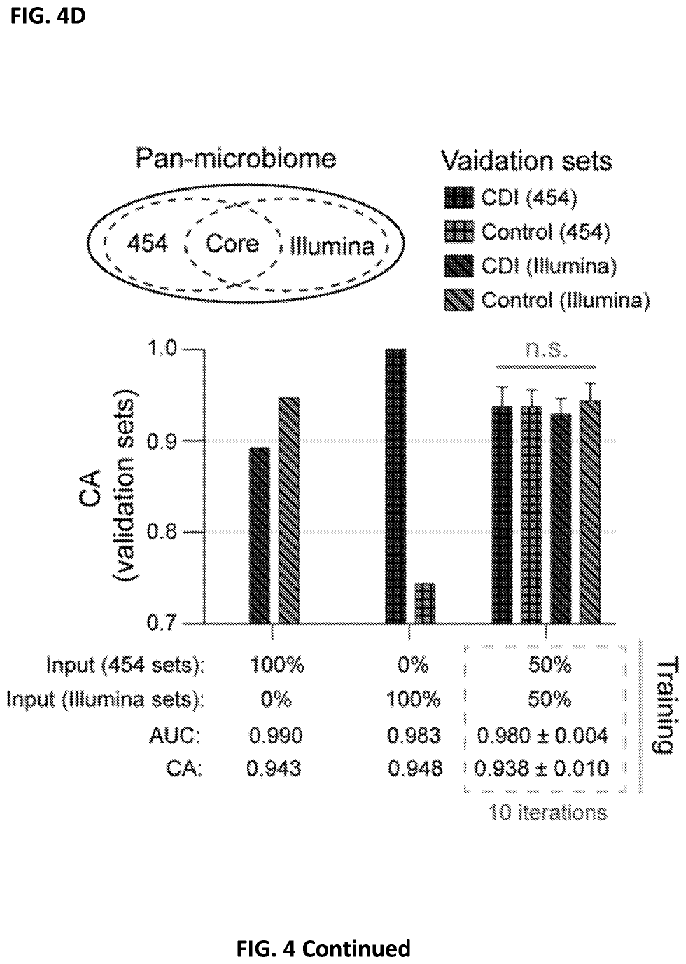

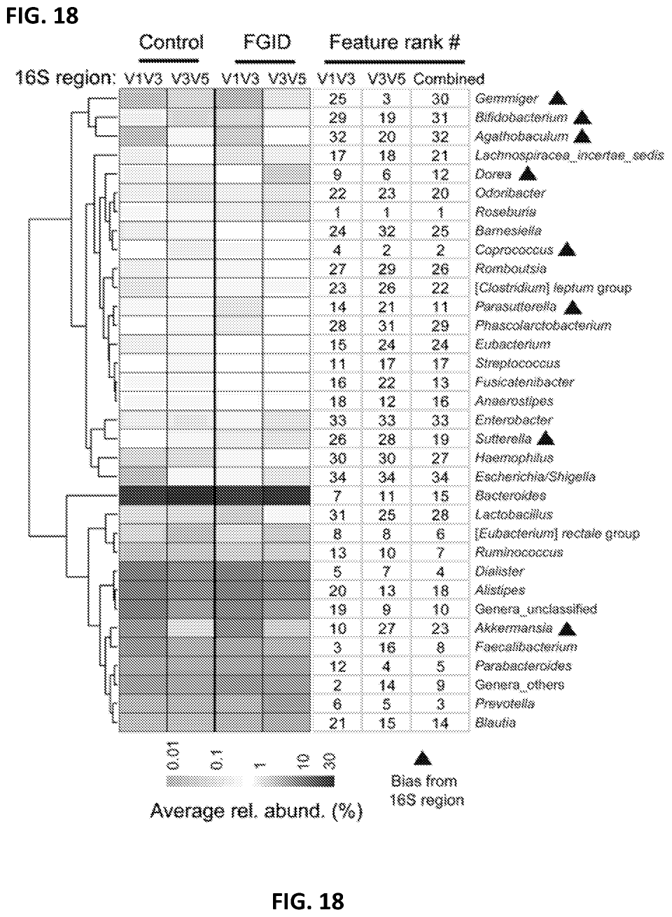

Disease classification is an important emerging application of gut microbiome surveys for biomarker discovery. Because the utility of pan-microbiome profiling had not yet been tested for disease classification, the inventors explored whether this approach improved classifier scores by merging core microbiome communities corrected for demographic and technical bias. In pilot studies using different sequencing modalities, the inventors benchmarked the center HMP cohort of pediatric FGID cases profiled by 16S VIV3 and V3V5 amplicons generated on the 454 pyrosequencing platform²¹. Analysis of β-diversity from collapsed Taxa4Meta taxonomy profiles did not separate FGID cases from healthy controls (FIG. 4A), and not surprisingly a suboptimal classification accuracy (CA<0.85) was evident when profiling individual VIV3 and V3V5 datasets (FIG. 4B). Using feature ranking generated by the random forest algorithm, the inventors selected >85% of genera abundance features that were common to both 16S regions, which identified previously underappreciated Roseburia as a core microbiome genus feature that discriminates between FGID and healthy controls (FIG. 18). Supervised training of pan-microbiome profiles significantly improved the classification accuracy when compared with individual microbiome surveys (FIG. 4B).

In further support, the inventors tested amplicon data generated from multiple CDI cohorts with sequence deposited from different 16S regions and technology platforms (Table 21). Unlike the subtle microbiome community differences observed in FGID cases. CDI patients present with consistent extreme dysbiosis that is evident across multiple sequencing strategies (FIG. 4C). Using a similar approach as above, platform-specific classification models performed well when differentiating CDI from Controls during training procedures, but failed to cross-validate subjects across different sequencing platforms and represents a major limitation for meta-analysis. This classification inaccuracy was eliminated by using pan-microbiome patterns for training (FIG. 4D), as it minimizes the impact of pattern variation and retains common microbiome features to facilitate biomarker discovery.

### Example 8—Utility of Pan-Microbiome Features for Diarrheal Disease Classification

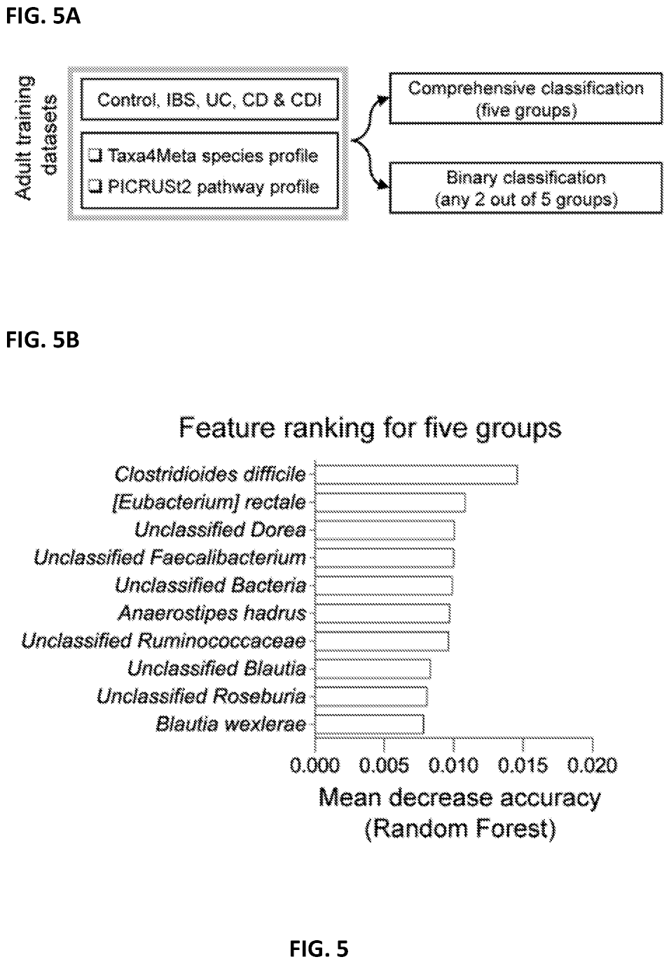

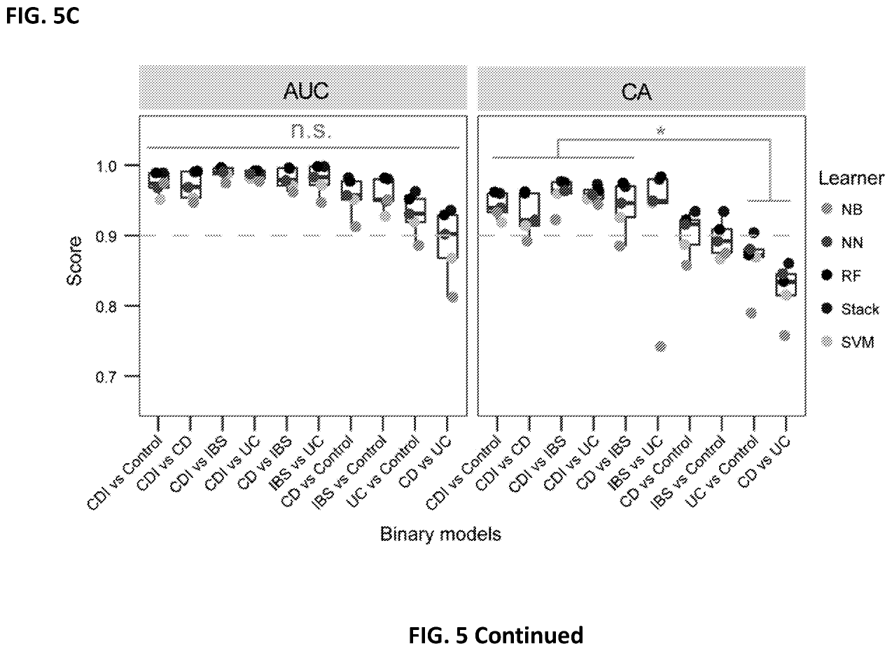

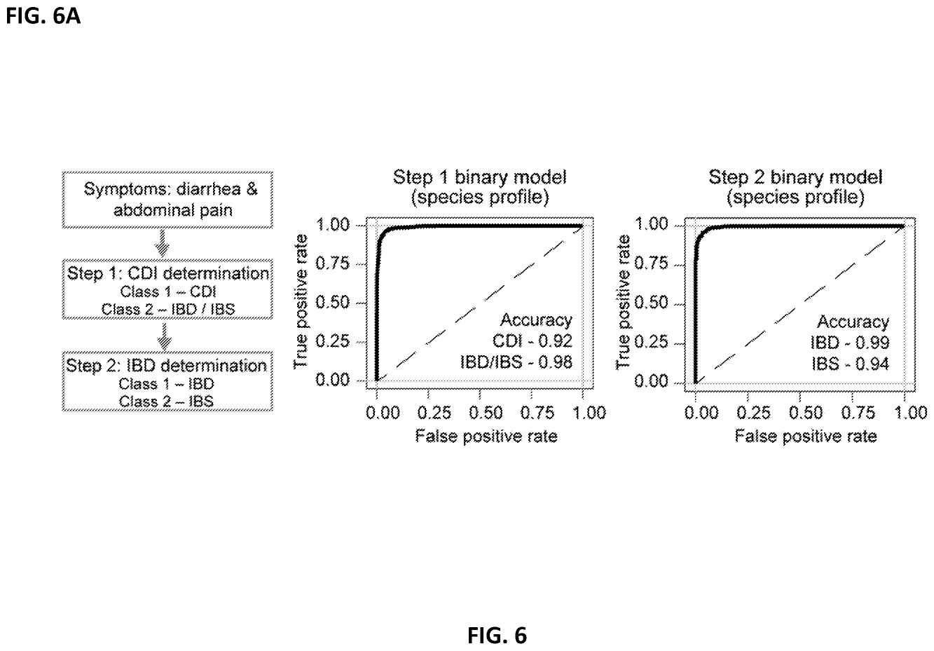

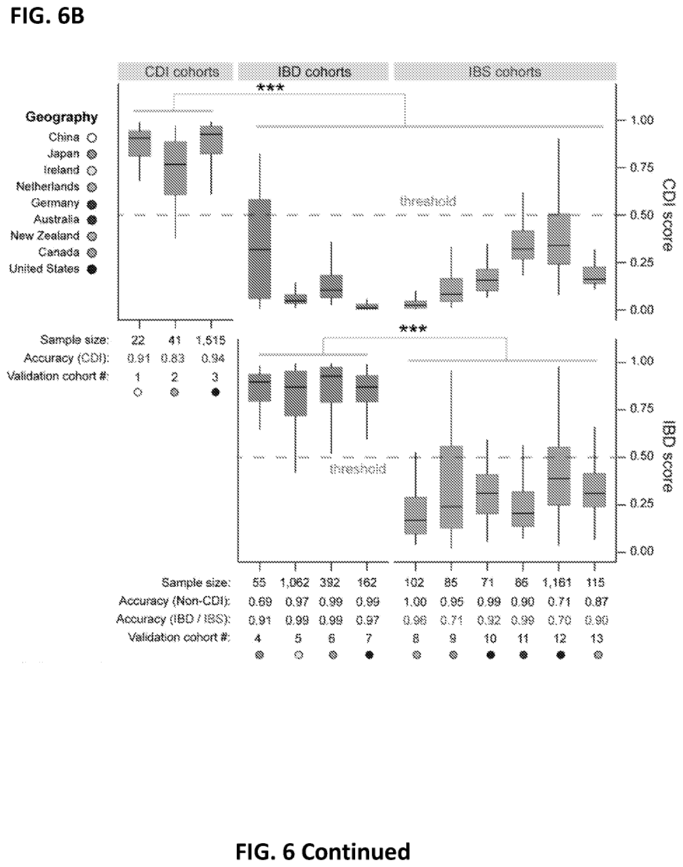

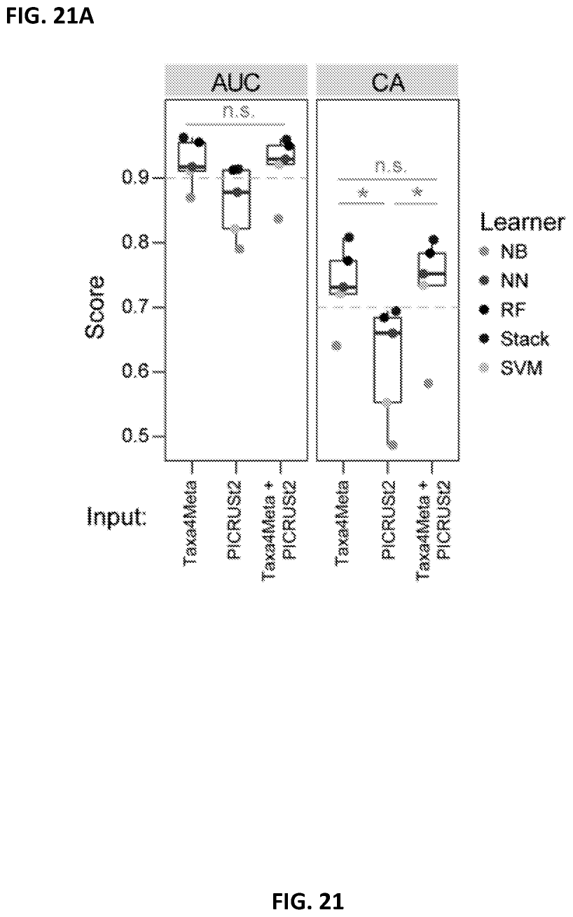

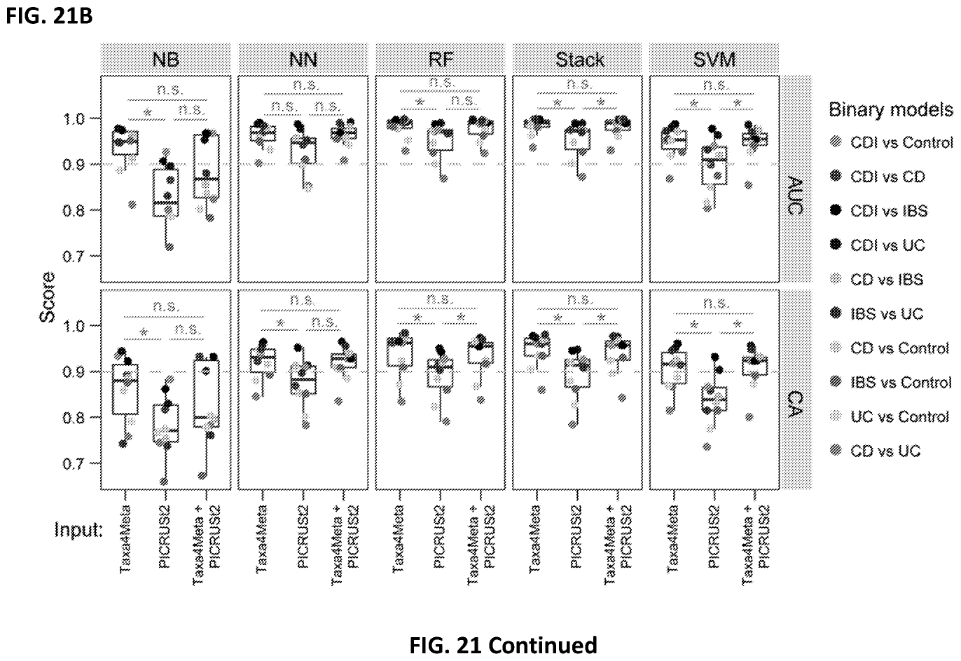

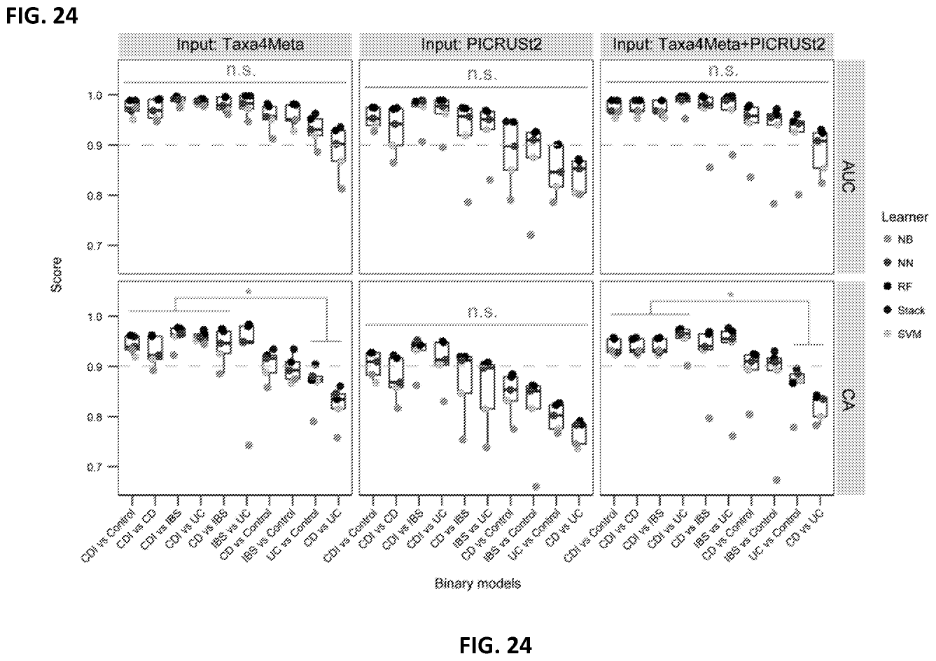

Two strategies were employed to generate comprehensive and binary disease classification models based on pan-microbiome profiles (FIG. 5A). Using Taxa4Meta taxonomic profiles, the inventors identified several key features including C. difficile (FIG. 5B) as the top discriminative feature that is pathophysiologically relevant in differentiating CDI from other diarrheal subtypes. This top-ranking feature was not identified as a classifier in two prior microbiome meta-analyses highlighting the technical bias in prior studies^{20,22}. Notably, the top Taxa4meta collapsed species significantly outperformed discriminative PICRUSt2 pathway features in comprehensive disease classification models (FIGS. 21A-21B). Taxa-based classification models for the five clinical groups investigated (Control. CDI. IBS. IBD UC and IBD CD) demonstrated excellent AUC results, but moderate CA scores indicating that disease classification across all cohort groups is suboptimal (FIG. 6C). This underperformance could be accounted for by the similarity of microbiome features in Control, UC, and IBS subjects, representing a challenge for reliable cross-classification (FIGS. 3A-3C). Nevertheless, in contrast to the multiple-group classification, binary models provided excellent disease classification with improved AUC and CA scores (FIG. 5C). Similar to the comprehensive classification models, binary disease classification using Taxa4meta collapsed species profiles outperformed discriminative functional pathways independent of learning algorithms used (FIGS. 21A-21B). Of note. CDI versus IBD/IBS/Control demonstrated consistently high classification accuracy (>0.9) at both taxonomic and functional levels (FIG. 24). These findings represent a significant advance in diarrheal disease classification, and highlight that compositionally distinct microbiome communities are discernable between infectious colitis (CDI) and IBD/IBS patients.

### Example 9—Prototypical Workflow for Clinical Diarrheal Disease Classification

With the urgent need to differentiate common symptoms in CDI. IBD and IBS, the inventors assembled a prototypical workflow to assist in stratifying these patients based on the disclosed Taxa4Meta-generated binary algorithms and/or features (FIG. 6A and Tables 1-17). Diagnosing CDI first was prioritized based on the clinical necessity for rapid treatment and patient contact-isolation. By applying a binary classifier that differentiated CDI from IBD and IBS cases, the inventors demonstrated a classification accuracy of 0.95 (FIG. 6A). A second binary classifier can then be applied that differentiated IBD from IBS cases with an overall classification accuracy of 0.96 (FIG. 6A). To independently validate the diagnostic workflow, the inventors tested 16S data generated from (1) recently published clinical CDI, IBD and IBS microbiome cohorts, and (2) real-world data obtained from self-reported IBD and IBS cases in the American Gut and LifeLines population cohorts. The identified features/classifiers correctly identified 93.6% of CDI patients, with the accuracy of 0.97 in differentiating clinically confirmed IBD versus IBS cases (FIG. 6B). These independent validation cohorts provide strong proof-of-concept data for pan-microbiome based classification in developing companion diagnostic workflows for diarrheal disease stratification.

### Example 10—Predictive Functional Analysis Highlights Disease-Specific Biochemical Pathways

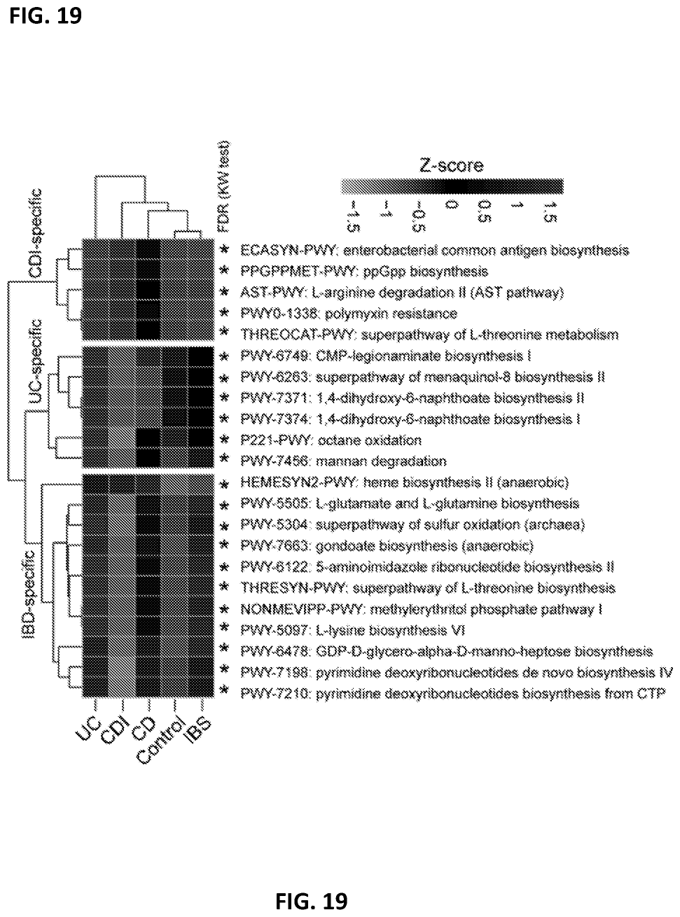

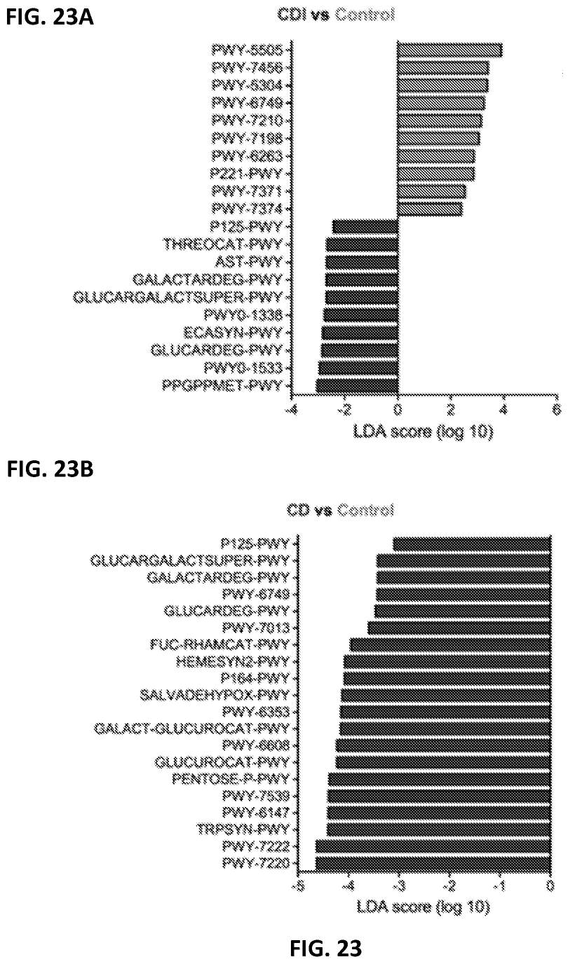

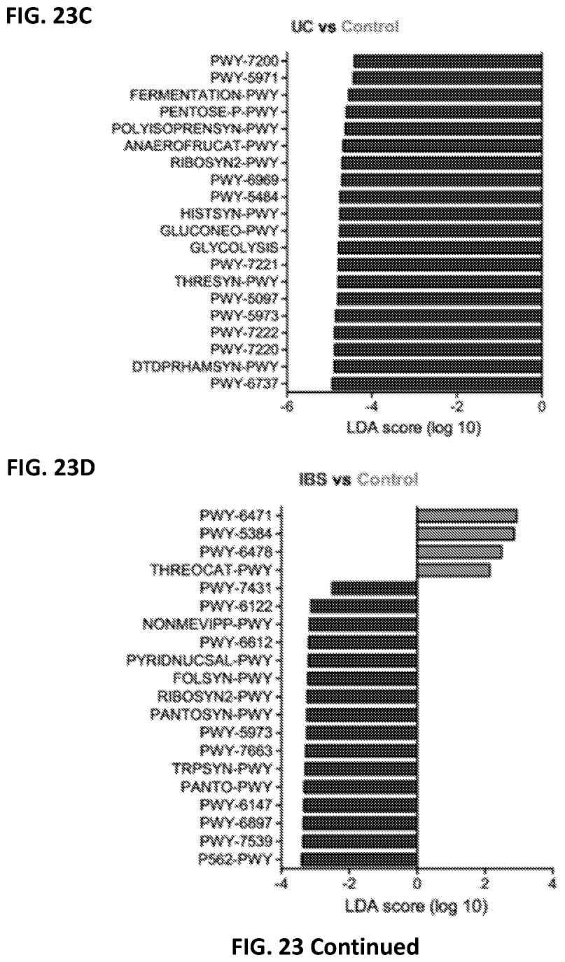

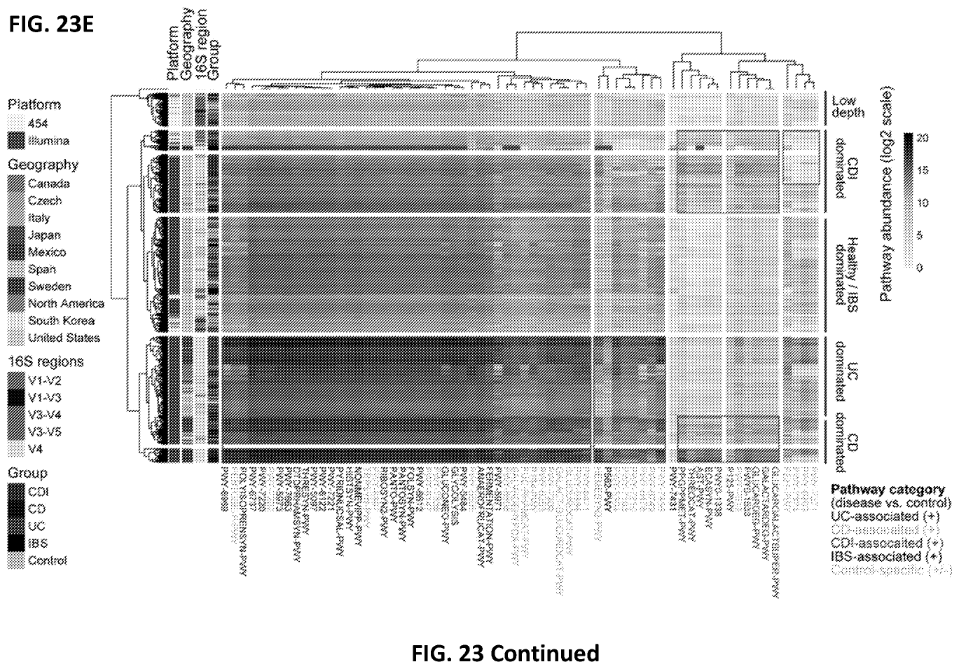

PICRUSt2 pathway profiles generated from Taxa4Meta OTU tables were used to characterize disease-specific core microbiome functions compared with healthy controls. Using a combination of LEfSe analysis and feature-scoring by the random forest algorithm, the inventors ranked biochemical pathways that were significantly associated with specific diarrheal disease types (FIGS. 23A-23E and Tables 1-8). The inventors identified a CDI-specific pathway cluster including ECASYN-PWY, PPGPPMET-PWY, PWY0-1338, AST-PWY and THREOCAT-PWY (FIG. 19). Polymyxin resistance (PWY0-1338) represented a strong CDI signature associated with pathobiome abundance, and is typically enriched after antibiotic exposure. Polymyxin is an antibiotic of last resort used to treat multi-drug resistant Gram-negative bacterial infections in patients who are later at high risk of developing CDI. Menaquinone biosynthetic pathways (PWY-7374, PWY-7371 and PWY-6263) were significantly underrepresented in CDI compared with Controls (FIG. 19 and FIGS. 23A-23E), reflecting the decreased abundance of healthy-associated Gram-positive bacteria in CDI, Menaquinones are important vitamin K2 based growth factors for many Gram-positive, obligate anaerobic gut bacteria. By contrast, these biosynthetic pathways were significantly elevated in IBC UC representing unique pathway signatures for IBD subtype classification (FIG. 19). Although beta-diversity distance profiles were not significant differentiated between IBD UC and controls (FIGS. 3A-3B), several other functional pathways were also significantly altered in IBD UC patients (FIG. 23C). Notably, simple saccharide catabolic pathways were highly enriched in IBD UC patients (Tables 1-8), which is supportive of clinical recommendations for IBD patients to avoid simple saccharides. These pathways are associated with increased disease severity in IL-10 deficient mice receiving dietary glucose and fructose. Nitrate reduction pathways were also significantly elevated in IBC UC patients (Tables 1-8), a function known to correlate with colitis risk in patients and IL-10 deficient mice. Dietary and host-derived nitrate also confers growth benefit to pathobiome expansion of Enterobacteriaceae in chemical-induced colitis. In bacteria, nitrate is reduced to nitrite followed by ammonia production, which ultimately serves as substrate for glutamate and glutamine biosynthesis as important carbon and nitrogen sources for gut microbiota. Thus, L-glutamate and L-glutamine biosynthesis (PWY-5505) is an important metabolic pathway for ammonia assimilation and this pathway was elevated in both IBD UC and IBD CD patients (FIG. 19), confirming previous reports linking clinical IBD disease severity with nitrogen reduction and ammonia production. In general, the findings are in agreement with several clinical and mouse models of nitrate metabolism by the IBD microbiota.

The disclosed meta-analysis also identified IBD-specific pathway clusters (FIG. 19 and FIG. 23E). For example, heme biosynthesis (HEMESYN2-PWY) was significantly enriched in IBD UC and IBD CD microbiomes, where heme supplementation is reported to exacerbate colitis in mouse and rat IBD models. Sulfur oxidation (PWY-5304) required by methanogenic archaca for sulfate production was also enriched in IBD-associated microbiome. In addition to the detection of methanogenic archaca retained as a feature for downstream analysis by Taxa4Meta, the IBD-associated microbiome is reported to be enriched in bacterial sulfate reducers, e.g. Desulfovibrio that convert sulfate to pathogenic hydrogen sulfide. Contrasting L-threonine metabolic pathways distinguished CDI from IBD-associated microbiomes, the former being enriched for catabolism (THREOCAT-PWY) whereas the latter facilitated biosynthesis (THRESYN-PWY) (FIG. 19). Increased threonine biosynthesis by IBD-associated microbiota has been implicated in impaired disease recovery, supported by L-threonine supplementation inhibiting mucosal healing in colitis models. Taken together, the utility of predictive metagenome functions based on population-scale microbiome profiles to exploit diarrheal-specific metabolic potential for biomarker discovery and disease classification is demonstrated.

### Example 11—Enterotypes Impact Disease Classification Performance

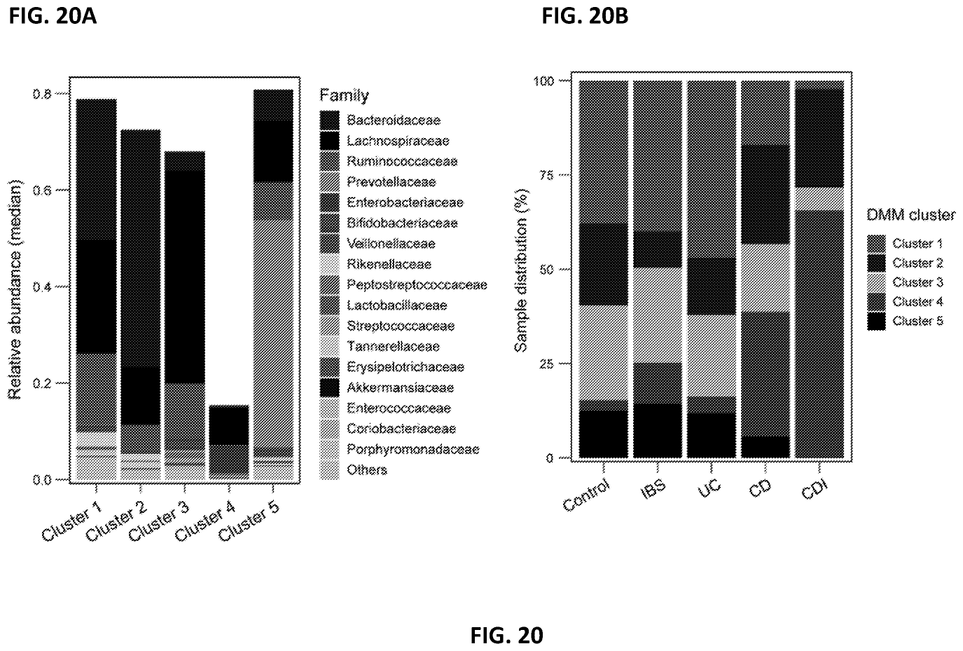

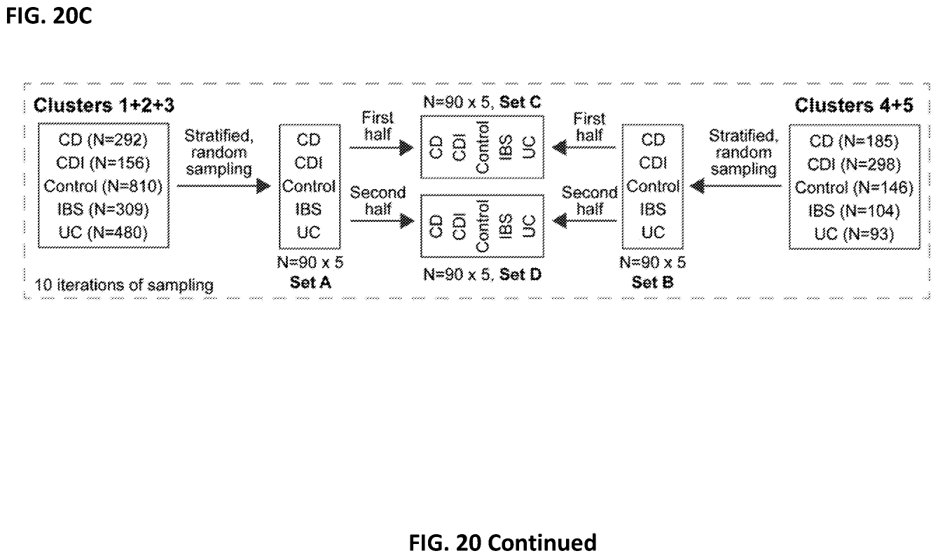

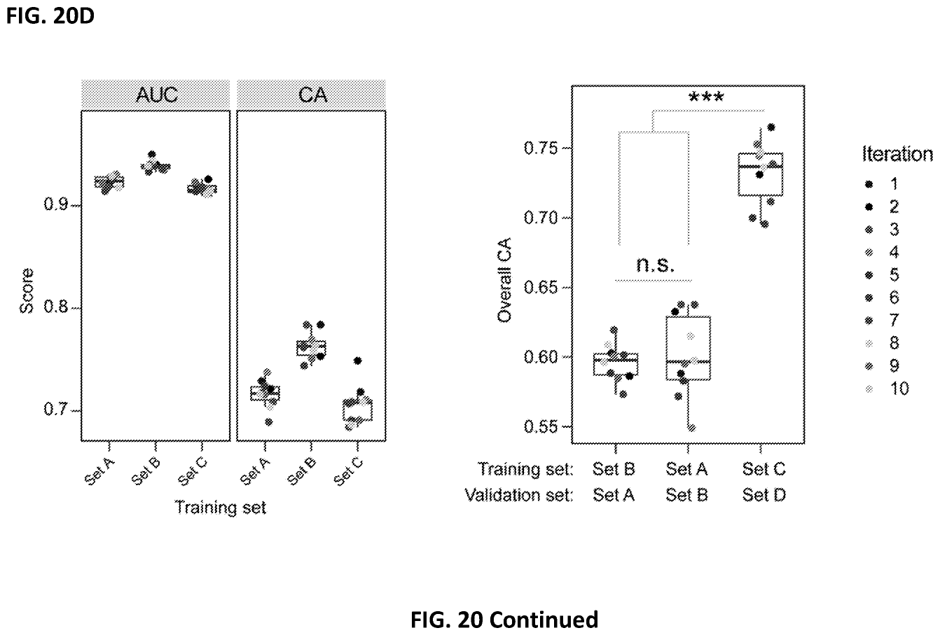

The population-scale meta-analysis described herein demonstrated the existence of different enterotypes that dominate across continents (FIG. 16), with three classical community types formed as previously reported. As demonstrated above, classification models constructed using pan-microbiome profile were expected to minimize enterotype bias. To test this, first a DMM algorithm for clustering all meta-analysis samples was applied, and identified five clusters (FIG. 20A). DMM clusters 1, 2 and 3, commonly observed in Control. IBS and UC (FIG. 20B), were easily distinguished from clusters 4 and 5 with the dominance of Enterobacteriaceae and Prevotellaceae, respectively. Next, the inventors investigated whether enterotype mismatching impacts classification performance. To minimize the classification variation introduced by small sample size, samples of DMM clusters 1, 2 and 3 were combined as one super-cluster, whereas DMM clusters 4 and 5 were combined as another super-cluster. To balance sample size for enterotype-specific comparisons, the inventors applied stratified, random sub-sampling procedure with a total of 10 iterations to sample subjects from two super-clusters to form cluster-specific dataset Sets A and B, and two pan-microbiome Sets C and D for downstream supervised classification analyses (FIG. 20C). While drastic differences in AUC and CA scores for either cluster-specific or pan-microbiome sample datasets were not observed, pan-microbiome disease classifiers demonstrated significantly improved CA scores during independent validation procedure when compared to cluster-specific disease classifiers (FIG. 20D). Although the analysis was not sufficiently powered to perform disease-specific comparisons, the findings are significant as they demonstrate that enterotype-bias represents an important consideration when developing universal microbiome-based disease classification models.

### REFERENCES

1. Quigley, E. M. M. Gut microbiome as a clinical tool in gastrointestinal disease management: Are we there yet? Nature Reviews Gastroenterology and Hepatology vol. 14 315-320 (2017).
2. Shanahan, F., Ghosh, T. S. & O'Toole, P. W. The Healthy Microbiome—What Is the Definition of a Healthy Gut Microbiome? Gastroenterology 160, 483-494 (2021).
3. Blaser, M., Bork, P., Fraser, C., Knight, R. & Wang, J. The microbiome explored: Recent insights and future challenges. Nature Reviews Microbiology vol. 11 213-217 (2013).
4. Duan, R., Zhu, S., Wang, B. & Duan, L. Alterations of gut microbiota in patients with irritable bowel syndrome based on 16s rRNA-targeted sequencing: A systematic review. Clinical and Translational Gastroenterology vol. 10 (2019).
5. Pittayanon, R. et al. Differences in Gut Microbiota in Patients With vs Without Inflammatory Bowel Diseases: A Systematic Review. Gastroenterology 158, 930-946.e1 (2020).
6. Iskandar, H. N. & Ciorba, M. A. Biomarkers in inflammatory bowel disease: Current practices and recent advances. Translational Research vol. 159 313-325 (2012).
7. Kim, J. H., Lin, E. & Pimentel, M. Biomarkers of irritable bowel syndrome. Journal of Neurogastroenterology and Motility vol. 23 20-26 (2017).
8. Wadhwa, A. et al. High risk of post-infectious irritable bowel syndrome in patients with Clostridium difficile infection. Aliment. Pharmacol. Ther. 44, 576-582 (2016).
9. Rao, K. & Higgins, P. D. R. Epidemiology, Diagnosis, and Management of Clostridium difficile Infection in Patients with Inflammatory Bowel Disease. Inflammatory Bowel Diseases vol. 22 1744-1754 (2016).
10. Murali, A., Bhargava, A. & Wright, E. S. IDTAXA: A novel approach for accurate taxonomic classification of microbiome sequences. Microbiome 6, (2018).
11. Gao, X., et al., A Bayesian taxonomic classification method for 16S rRNA gene sequences with improved species-level accuracy. BMC Bioinformatics 18, (2017).
12. Whon, T. W. et al. Data Descriptor: The effects of sequencing platforms on phylogenetic resolution in 16 S rRNA gene profiling of human feces. Sci. Data 5, (2018).
13. McIntyre, A. B. R. et al. Comprehensive benchmarking and ensemble approaches for metagenomic classifiers. Genome Biol. 18, (2017).
14. Truong, D. T. et al. MetaPhlAn2 for enhanced metagenomic taxonomic profiling. Nature Methods vol. 12 902-903 (2015).
15. McDonald, D. et al. American Gut: an Open Platform for Citizen Science Microbiome Research. mSystems 3, (2018).
16. Tigchelaar, E. F. et al. Cohort profile: LifeLines DEEP, a prospective, general population cohort study in the northern Netherlands: Study design and baseline characteristics. BMJ Open 5, (2015).
17. Scher, J. U. et al. Expansion of intestinal Prevotella copri correlates with enhanced susceptibility to arthritis. Elife 2013, (2013).
18. Wu, Q. & Savidge, T. C. Systems approaches for the clinical diagnosis of Clostridioides difficile infection. Translational Research vol. 220 57-67 (2020).
19. Jeffery, I. B. et al. Differences in Fecal Microbiomes and Metabolomes of People With vs Without Irritable Bowel Syndrome and Bile Acid Malabsorption. Gastroenterology 158, 1016-1028.e8 (2020).
20. Duvallet, C., et al., Meta-analysis of gut microbiome studies identifies disease-specific and shared responses. Nat. Commun. 8, (2017).
21. Saulnier, D. M. et al. Gastrointestinal microbiome signatures of pediatric patients with irritable bowel syndrome. Gastroenterology 141, 1782-1791 (2011).
22. Mancabelli, L. et al. Identification of universal gut microbial biomarkers of common human intestinal diseases by meta-analysis. FEMS Microbiol. Ecol. 93, fix153 (2017).
23. Wang, J. et al. Meta-analysis of human genome-microbiome association studies: The MiBioGen consortium initiative. Microbiome 6, (2018).
24. Vich Vila, A. et al. Gut microbiota composition and functional changes in inflammatory bowel disease and irritable bowel syndrome. Sci. Transl. Med. 10, eaap8914 (2018).
25. Gevers, D. et al. The treatment-naive microbiome in new-onset Crohn's disease. Cell Host Microbe 15, 382-392 (2014).
26. Clooney, A. G. et al. Ranking microbiome variance in inflammatory bowel disease: A large longitudinal intercontinental study. Gut 70, 499-510 (2021).
27. Hillmann, B. et al. Evaluating the Information Content of Shallow Shotgun Metagenomics. mSystems 3, (2018).
28. Cole, J. R. et al. Ribosomal Database Project: Data and tools for high throughput rRNA analysis. Nucleic Acids Res. 42, (2014).
29. Martin, M. Cutadapt removes adapter sequences from high-throughput sequencing reads. EMBnet.journal 17, 10 (2011).
30. Edgar, R. C. Search and clustering orders of magnitude faster than BLAST. Bioinformatics 26, 2460-2461 (2010).
31. Rognes, T., et al., F. VSEARCH: A versatile open source tool for metagenomics. PeerJ 2016, (2016).
32. Callahan, B. J. et al. DADA2: High-resolution sample inference from Illumina amplicon data. Nat. Methods 13, 581-583 (2016).
33. Demšar, J. et al. Orange: Data mining toolbox in python. J. Mach. Learn. Res. 14, 2349-2353 (2013).

1) A method of treating an individual having diarrhea comprising:
measuring for one or more taxonomical features from a biological sample from the individual; and
reducing the administration of antibiotics and/or antimicrobial treatment to the individual when the individual has presence or absence or a certain level of one or more feature(s) indicative of non-CDI causative diarrhea, or
administering antibiotics and/or antimicrobial treatment to the individual when the individual has presence or absence or a certain level of one or more feature(s) indicative of pathogenic infection.

2) The method of claim 1, wherein the antibiotics and/or antimicrobial treatment comprise at least one of the antibiotics selected from a small molecule antibiotic, an antibiotic derived from a natural product, a microbial composition, an antibody suitable for neutralizing pathogenic infections, a therapeutic, contact isolation, and any combination thereof.

3) The method of claim 2, with the proviso that if the non-CDI causative diarrhea is irritable bowel syndrome (IBS), administration of the antibiotic and/or antimicrobial rifaximin is not reduced.

4) The method of claim 2, wherein the antibiotics and/or antimicrobial treatment comprises at least one of vancomycin, fidaxomicin, and bezlotoxumab.

5) The method of claim 4, wherein the treatment is fidaxomicin, and optionally the treatment dosage is at least 200 mg twice daily for 10 days, the treatment is vancomycin, and optionally the treatment dosage is at least 125 mg four times per day for 10 days, and/or the treatment is bezlotoxumab.

6) The method of claim 1, wherein the pathogenic diarrhea classification or non-CDI causative diarrhea classification is characterized by measuring the presence, absence, and/or relative quantity of at least 10 taxonomical features described in any one of Tables 9-17.

7) The method of claim 6, wherein the pathogenic diarrhea classification or non-CDI causative diarrhea classification is characterized by measuring the presence, absence, and/or relative quantity of at least 20 taxonomical features described in any one of Tables 9-17.

8) The method of claim 6, wherein the pathogenic diarrhea classification or non-CDI causative diarrhea classification is characterized by measuring the presence, absence, and/or relative quantity of at least 40 taxonomical features described in any one of Tables 9-17.

9) The method of claim 6, wherein the pathogenic diarrhea classification or non-CDI causative diarrhea classification is characterized by measuring the presence, absence, and/or relative quantity of at least 60 taxonomical features described in any one of Tables 9-17.

10) The method of claim 6, wherein the pathogenic diarrhea classification or non-CDI causative diarrhea classification is characterized by measuring the presence, absence, and/or relative quantity of at least 80 taxonomical features described in any one of Tables 9-17.

11) The method of claim 6, wherein the pathogenic diarrhea classification or non-CDI causative diarrhea classification is characterized by measuring the presence, absence, and/or relative quantity of at least 100 taxonomical features described in any one Tables 9-17.

12) The method of any one of claims 7-11, wherein the pathogenic diarrhea classification or non-CDI causative diarrhea classification comprises characterization using more than one of Tables 9-17.

13) The method of claim 12, wherein the more than one characterization using Tables 9-17 is sequential.

14) The method of claim 12, wherein the more than one characterization using Tables 9-17 comprises first characterizing using Tables 10 and/or 12, followed by characterization using one or more of the remaining Tables.

15) The method of claim 12, wherein the measuring of one or more taxonomical features comprises at least one of analyzing one or more nucleic acids in the sample, analyzing one or more metabolites in the sample, and analyzing one or more proteins in the sample.

16) The method of claim 15, wherein the nucleic acid is analyzed by sequencing, polymerase chain reaction, isothermal amplification, bioinformatics, or any combination thereof.

17) The method of claim 16, wherein the nucleic acid analyzed is 16S ribosomal RNA.

18) The method of claim 15, wherein the metabolites are analyzed by mass spectrometry, ELISA, chromatography, or any combination thereof.

19) The method of claim 15, wherein the proteins are analyzed by mass spectrometry, ELISA, chromatography, Western blotting, immunoprecipitation, immunoelectrophoresis, or any combination thereof.

20) The method of claim 1, wherein when reducing the administration of antibiotics and/or antimicrobial treatment to the individual when the individual has presence or absence or a certain level of one or more feature(s) indicative of non-CDI causative diarrhea, the subject microbiome is further characterized to determine whether the non-CDI causative diarrhea is associated with irritable bowel syndrome (IBS) or inflammatory bowel disease (IBD), and treatment is modified accordingly.

21) The method of claim 20, wherein the non-CDI causative diarrhea is associated with IBD, the IBD is further characterized to determine whether the IBD is Ulcerative Colitis (UC) or Crohn's Disease (CD), and treatment is modified accordingly.

22) The method of claim 1, wherein the measuring of one or more taxonomical features from a biological sample from the individual comprises determining changes in relative abundance, compared to a reference healthy gut microbiome, of at least one of:
Sphingobacteriaceae Pedobacter; Saccharibacteria Incertae Sedis; Peptostreptococcaceae Intestinibacter; Bacillales Incertae Sedis Gemella; Veillonella infantium; Chloroplast Streptophyta; Caulobacter segnis; Methylophilaceae Methylophilus; Lactobacilluses Streptococcaceae; Burkholderiales Comamonadaceae; Burkholderia ambifaria; Caulobacteraceae Caulobacter; Betaproteobacteria; Phyllobacteriaceae Phyllobacterium; Burkholderiales Burkholderiaceae; Sphingomonadaceae; Sphingomonas; Prevotellamassilia timonensis; Collinsella aerofaciens; Adlercreutzia equolifaciens; Blautia hominis; and Dorea formicigenerans;
wherein the change in taxonomical feature relative abundance is indicative of non-CDI causative diarrhea associated with IBS.

23) The method of claim 22, wherein an increase in relative abundance of at least one of:
Sphingobacteriaceae Pedobacter; Saccharibacteria Incertae Sedis; Peptostreptococcaceae Intestinibacter; Bacillales Incertae Sedis Gemella; Veillonella infantium; Chloroplast Streptophyta; Caulobacter segnis; Methylophilaceae Methylophilus; Lactobacillus Streptococcaceae; Burkholderiales Comamonadaceae; Burkholderia ambifaria; Caulobacteraceae Caulobacter; Betaproteobacteria; Phyllobacteriaceae Phyllobacterium; Burkholderiales Burkholderiaceae; Sphingomonadaceae; Sphingomonas; and Prevotellamassilia timonensis, is indicative of non-CDI causative diarrhea associated with IBS.

24) The method of claim 22, wherein a decrease in relative abundance of at least one of:
Collinsella aerofaciens; Adlercreutzia equolifaciens; Blautia hominis; and Dorea formicigenerans, is indicative of non-CDI causative diarrhea associated with IBS.

25) The method of any one of claims 22-24, wherein a change in relative abundance of at least four taxonomical features is indicative of non-CDI causative diarrhea associated with IBS.

26) The method of claim 1, wherein the measuring of one or more taxonomical features from a biological sample from the individual comprises determining changes in relative abundance, compared to a reference healthy gut microbiome, of at least one of:
Acidaminococcaceae Acidaminococcus; Actinomycetales Actinomycetaceae; Bacillales Gemella; Bacilli Lactobacillus; Betaproteobacteria; Betaproteobacteria Burkholderiales; Bifidobacterium boum; Blautia hominis; Clostridiales Incertae Sedis XI Parvimonas; Enterobacteriaceae Cedecea; Erysipelotrichaceae Faecalicoccus; Faecalimonas umbilicata; Fusobacteriaceae Fusobacterium; Fusobacterium nucleatum; Gammaproteobacteria Enterobacterales; Lachnoclostridium [Clostridium] bolteae; Lachnospiraceae Lachnoclostridium; Megasphaera micronuciformis; Peptoniphilaceae Peptoniphilus; Peptostreptococcaceae Peptostreptococcus; Peptostreptococcaceae Romboutsia; Peptostreptococcaceae Terrisporobacter; Proteobacteria Gammaproteobacteria; Rosenbergiella collisarenosi; Rosenbergiella nectarea; Ruminococcaceae Intestinimonas; Ruminococcaceae Subdoligranulum; Solobacterium moorei; Veillonellaceae Veillonella; Alistipes shahii; Bacteroides cellulosilyticus; Bacteroides koreensis; Bacteroides thetaiotaomicron; Bacteroides xylanisolvens; Colidextribacter massiliensis; Coprococcus catus; Coprococcus eutactus; Enterobacterales Enterobacteriaceae; Faecalibacterium prausnitzii; Methanobrevibacter smithii; Phascolarctobacterium faecium; Romboutsia timonensis; and Turicibacter sanguinis;
wherein the change in taxonomical feature relative abundance is indicative of non-CDI causative diarrhea associated with IBD.

27) The method of claim 26, wherein an increase in relative abundance of at least one of:
Acidaminococcaceae Acidaminococcus; Actinomycetales Actinomycetaceae; Bacillales Gemella; Bacilli Lactobacillus; Betaproteobacteria; Betaproteobacteria Burkholderiales; Bifidobacterium boum; Blautia hominis; Clostridiales Incertae Sedis XI Parvimonas; Enterobacteriaceae Cedecea; Erysipelotrichaceae Faecalicoccus; Faecalimonas umbilicata; Fusobacteriaceae Fusobacterium; Fusobacterium nucleatum; Gammaproteobacteria Enterobacterales; Lachnoclostridium [Clostridium] bolteae; Lachnospiraceae Lachnoclostridium; Megasphaera micronuciformis; Peptoniphilaceae Peptoniphilus; Peptostreptococcaceae Peptostreptococcus; Peptostreptococcaceae Romboutsia; Peptostreptococcaceae Terrisporobacter; Proteobacteria Gammaproteobacteria; Rosenbergiella collisarenosi; Rosenbergiella nectarea; Ruminococcaceae Intestinimonas; Ruminococcaceae Subdoligranulum; Solobacterium moorei; and Veillonellaceae Veillonella, is indicative of non-CDI causative diarrhea associated with IBD.

28) The method of claim 26, wherein a decrease in relative abundance of at least one of:
Alistipes shahii; Bacteroides cellulosilyticus; Bacteroides koreensis; Bacteroides thetaiotaomicron; Bacteroides xylanisolvens; Colidextribacter massiliensis; Coprococcus catus; Coprococcus eutactus; Enterobacterales Enterobacteriaceae; Faecalibacterium prausnitzii; Methanobrevibacter smithii; Phascolarctobacterium faecium; Romboutsia timonensis; and Turicibacter sanguinis, is indicative of non-CDI causative diarrhea associated with IBD.

29) The method of any one of claims 26-28, wherein a change in relative abundance of at least four taxonomical features is indicative of non-CDI causative diarrhea associated with IBD.

30) The method of claim 1, wherein the individual is a pediatric individual, and wherein the measuring of one or more taxonomical features from a biological sample from the individual comprises determining changes in relative abundance, compared to a reference healthy pediatric gut microbiome, of at least one of:
Bacilli Lactobacillus; Peptostreptococcaceae Clostridioides; Enterococcaceae Enterococcus; Eggerthellaceae Eggerthella; Erysipelotrichaceae Erysipelatoclostridium; Lachnospiraceae Lachnoclostridium; Firmicutes Bacilli; Enterobacteriaceae Escherichia; Enterobacteriales Enterobacteriaceae; Streptococcaceae Streptococcus; Actinomycetaceae Schaalia; Clostridiaceae Hungatella; Clostridiaceae Clostridium; Corynebacteriaceae Corynebacterium; Veillonellaceae Veillonella; Actinomycetaceae Actinomyces; Fusobacteriaceae Fusobacterium; Erysipelotrichaceae Longicatena; Lachnospiraceae Clostridium XlVa; Lachnospiraceae Eisenbergiella; Peptostreptococcaceae Intestinibacter; Enterobacteriaceae Citrobacter; Peptoniphilaceae Finegoldia; Carnobacteriaceae Granulicatella; Coriobacteriaceae Eggerthella; Peptostreptococcaceae Peptostreptococcus; Drancourtella massiliensis; Peptoniphilaceae Anaerococcus; Bacillales Gemella; Lachnospiraceae Sellimonas; Peptoniphilaceae Peptoniphilus; Pasteurellaceae Rodentibacter; Clostridium sensu stricto; Lachnospiraceae Faecalimonas; Proteobacteria Gammaproteobacteria; Clostridiaceae Lactonifactor; Micrococcaceae Rothia; Leuconostocaceae Weissella; Peptostreptococcaceae Terrisporobacter; Peptostreptococcaceae Romboutsia; Peptostreptococcaceae Clostridium XI; Coriobacteriaceae Atopobium; Atopobiaceae Atopobium; Ruminococcaceae Anaeromassilibacillus; Porphyromonadaceae Parabacteroides; Rikenellaceae Alistipes; Lachnospiraceae Eubacterium rectale group; Lachnospiraceae Coprococcus; Lachnospiraceae Roseburia; Porphyromonadacede Odoribacter; Veillonellaceae Dialister; Ruminococcaceae Clostridium IV; Bacteroidia Bacteroidales; Coriobacteriaceae Collinsella; Coriobacteriales Coriobacteriaceae; Ruminococcaceae Clostridium leptum group; Enterobacteriaceae Shimwellia; Acidaminococcaceae Phascolarctobacterium; Staphylococcaceae Staphylococcus; Lachnospiraceae Anaerobutyricum; Erysipelotrichaceae Holdemania; Clostridiales Monoglobus; Porphyromonadaceae Barnesiella; Pasteurellales Pasteurellaceae; Clostridiales Clostridiaceae 1; and Bacteroidales Rikenellaceae;
wherein the change in taxonomical feature relative abundance is indicative of CDI associated diarrhea.

31) The method of claim 30, wherein an increase in relative abundance of at least one of:
Bacilli Lactobacillus; Peptostreptococcaceae Clostridioides; Enterococcaceae Enterococcus; Eggerthellaceae Eggerthella; Erysipelotrichaceae Erysipelatoclostridium; Lachnospiraceae Lachnoclostridium; Firmicutes Bacilli; Enterobacteriaceae Escherichia; Enterobacteriales Enterobacteriaceae; Streptococcaceae Streptococcus; Actinomycetaceae Schaalia; Clostridiaceae Hungatella; Clostridiaceae Clostridium; Corynebacteriaceae Corynebacterium; Veillonellaceae Veillonella; Actinomycetaceae Actinomyces; Fusobacteriaceae Fusobacterium; Erysipelotrichaceae Longicatena; Lachnospiraceae Clostridium XIVa; Lachnospiraceae Eisenbergiella; Peptostreptococcaceae Intestinibacter; Enterobacteriaceae Citrobacter; Peptoniphilaceae Finegoldia; Carnobacteriaceae Granulicatella; Coriobacteriaceae Eggerthella; Peptostreptococcaceae Peptostreptococcus; Drancourtella massiliensis; Peptoniphilaceae Anaerococcus; Bacillales Gemella; Lachnospiraceae Sellimonas; Peptoniphilaceae Peptoniphilus; Pasteurellaceae Rodentibacter; Clostridium sensu stricto; Lachnospiraceae Faecalimonas; Proteobacteria Gammaproteobacteria; Clostridiaceae Lactonifactor; Micrococcaceae Rothia; Leuconostocaceae Weissella; Peptostreptococcaceae Terrisporobacter; Peptostreptococcaceae Romboutsia; Peptostreptococcaceae Clostridium XI; Coriobacteriaceae Atopobium; Atopobiaceae Atopobium; and Ruminococcaceae Anaeromassilibacillus, is indicative of CDI associated diarrhea.

32) The method of claim 30, wherein a decrease in relative abundance of at least one of:
Porphyromonadaceae Parabacteroides; Rikenellaceae Alistipes; Lachnospiraceae Eubacterium rectale group; Lachnospiraceae Coprococcus; Lachnospiraceae Roseburia; Porphyromonadacede Odoribacter; Veillonellaceae Dialister; Ruminococcaceae Clostridium IV; Bacteroidia Bacteroidales; Coriobacteriaceae Collinsella; Coriobacteriales Coriobacteriaceae; Ruminococcaceae Clostridium leptum group; Enterobacteriaceae Shimwellia; Acidaminococcaceae Phascolarctobacterium; Staphylococcaceae Staphylococcus; Lachnospiraceae Anaerobutyricum; Erysipelotrichaceae Holdemania; Clostridiales Monoglobus; Porphyromonadaceae Barnesiella; Pasteurellales Pasteurellaceae; Clostridiales Clostridiaceae 1; and Bacteroidales Rikenellaceae, is indicative of CDI associated diarrhea.

33) The method of any one of claims 30-32, wherein a change in relative abundance of at least four taxonomical features is indicative of CDI associated diarrhea.

34) The method of claim 20, wherein the characterization of the subject microbiome to determine whether the non-CDI causative diarrhea is associated with IBS or IBD comprises measuring one or more taxonomical features from a biological sample from the subject and determining changes in relative abundance, compared to a reference dysbiosis IBD gut microbiome, of at least one of:
Blautia stercoris; Saccharibacteria Incertae Sedis; Bacteroides plebeius; Bacteroides nordii; Eubacterium siraeum; Bacteroides cellulosilyticus; Burkholderiales Burkholderiaceae; Caulobacteraceae Caulobacter; Pelomonas aquatic; Burkholderiaceae Burkholderia; Caulobacter segnis; Burkholderia ambifaria; Eubacterium ventriosum; Firmicutes Clostridia; Oxalobacter formigenes; Burkholderiales Comamonadaceae; Veillonella infantium; Bacteroides thetaiotaomicron; Sphingobacteriaceae Pedobacter; Burkholderia thailandensis; Erysipelotrichaceae Turicibacter; Collinsella aerofaciens; Veillonellaceae Veillonella; Lachnospiraceae Lachnoclostridium; Blautia hominis; Gammaproteobacteria Enterobacterales; Bifidobacteriales Bifidobacteriaceae; Ruminococcaceae Intestinimonas; Faecalimonas umbilicata; Actinomycetales Actinomycetaceae; Actinobacteria Actinobacteria; Dorea formicigenerans; and Bacteria Proteobacteria;
wherein the change in taxonomical feature relative abundance is indicative of non-CDI causative diarrhea associated with IBS.

35) The method of claim 34, wherein an increase in relative abundance of at least one of:
Saccharibacteria Incertae Sedis; Bacteroides plebeius; Bacteroides nordii; Eubacterium siraeum; Bacteroides cellulosilyticus; Burkholderiales Burkholderiaceae; Caulobacteraceae Caulobacter; Pelomonas aquatic; Burkholderiaceae Burkholderia; Caulobacter segnis; Burkholderia ambifaria; Eubacterium ventriosum; Firmicutes Clostridia; Oxalobacter formigenes; Burkholderiales Comamonadaceae; Veillonella infantium; Bacteroides thetaiotaomicron; Sphingobacteriaceae Pedobacter; Burkholderia thailandensis; and Erysipelotrichaceae Turicibacter, is indicative of non-CDI causative diarrhea associated with IBS.

36) The method of claim 34, wherein a decrease in relative abundance of at least one of:
Collinsella aerofaciens; Veillonellaceae Veillonella; Lachnospiraceae Lachnoclostridium; Blautia hominis; Gammaproteobacteria Enterobacterales; Bifidobacteriales Bifidobacteriaceae; Ruminococcaceae Intestinimonas; Faecalimonas umbilicata; Actinomycetales Actinomycetaceae; Actinobacteria Actinobacteria; Dorea formicigenerans; and Bacteria Proteobacteria, is indicative of non-CDI causative diarrhea associated with IBS.

37) The method of any one of claims 34-36, wherein a change in relative abundance of at least four taxonomical features is indicative of non-CDI causative diarrhea associated with IBS.

38) The method of claim 21, wherein the characterization of the subject microbiome to determine whether the non-CDI causative diarrhea is associated with IBD UC or IBD CD comprises measuring one or more taxonomical features from a biological sample from the subject and determining changes in relative abundance, compared to a reference healthy gut microbiome, of at least one of:
Lachnospiraceae Lachnoclostridium; Ruminococcaceae Intestinimonas; Acidaminococcaceae Acidaminococcus; Bacilli Lactobacillus; Actinomycetales Actinomycetaceae; Ruminococcaceae Subdoligranulum; Peptostreptococcaceae Romboutsia; Bifidobacterium boum; Bacillales Gemella; Peptostreptococcaceae Peptostreptococcus; Peptoniphilaceae Peptoniphilus; Clostridiales Incertae Sedis XI Parvimonas; Peptostreptococcaceae Terrisporobacter; Erysipelotrichaceae Faecalicoccus; Solobacterium moorei; Bacteroides xylanisolvens; Enterobacterales Enterobacteriaceae; Bacteroides koreensis; Bacteroides cellulosilyticus; Phascolarctobacterium faecium; and Bacteroides thetaiotaomicron, wherein the change in taxonomical feature relative abundance is indicative of non-CDI causative diarrhea associated with IBD UC.

39) The method of claim 38, wherein an increase in relative abundance of at least one of:
Lachnospiraceae Lachnoclostridium; Ruminococcaceae Intestinimonas; Acidaminococcaceae Acidaminococcus; Bacilli Lactobacillus; Actinomycetales Actinomycetaceae; Ruminococcaceae Subdoligranulum; Peptostreptococcaceae Romboutsia; Bifidobacterium boum; Bacillales Gemella; Peptostreptococcaceae Peptostreptococcus; Peptoniphilaceae Peptoniphilus; Clostridiales Incertae Sedis XI Parvimonas; Peptostreptococcaceae Terrisporobacter; Erysipelotrichaceae Faecalicoccus; and Solobacterium moorei, is indicative of non-CDI causative diarrhea associated with IBD UC.

40) The method of claim 38, wherein a decrease in relative abundance of at least one of:
Bacteroides xylanisolvens; Enterobacterales Enterobacteriaceae; Bacteroides koreensis; Bacteroides cellulosilyticus; Phascolarctobacterium faecium; and Bacteroides thetaiotaomicron, is indicative of non-CDI causative diarrhea associated with IBD UC.

41) The method of any one of claims 38-40, wherein a change in relative abundance of at least four taxonomical features is indicative of non-CDI causative diarrhea associated with IBD UC.

42) The method of claim 21, wherein the characterization of the subject microbiome to determine whether the non-CDI causative diarrhea is associated with IBD UC or IBD CD comprises measuring one or more taxonomical features from a biological sample from the subject and determining changes in relative abundance, compared to a reference healthy gut microbiome, of at least one of:
Faecalimonas umbilicata; Lachnospiraceae Lachnoclostridium; Lachnoclostridium [Clostridium] bolteae; Proteobacteria Gammaproteobacteria; Bacilli Lactobacillus; Actinomycetales Actinomycetaceae; Gammaproteobacteria Enterobacterales; Rosenbergiella collisarenosi; Rosenbergiella nectarea; Veillonellaceae Veillonella; Acidaminococcaceae Acidaminococcus; Blautia hominis; Bifidobacterium boum; Enterobacteriaceae Cedecea; Ruminococcaceae Intestinimonas; Peptostreptococcaceae Romboutsia; Fusobacteriaceae Fusobacterium; Fusobacterium nucleatum; Megasphaera micronuciformis; Romboutsia timonensis; Faecalibacterium prausnitzii; Coprococcus catus; Alistipes shahii; Bacteroides cellulosilyticus; Bacteroides thetaiotaomicron; Coprococcus eutactus; Turicibacter sanguinis; Colidextribacter massiliensis; and Methanobrevibacter smithii;
wherein the change in taxonomical feature relative abundance is indicative of non-CDI causative diarrhea associated with IBD CD.

43) The method of claim 42, wherein an increase in relative abundance of at least one of:
Faecalimonas umbilicata; Lachnospiraceae Lachnoclostridium; Lachnoclostridium [Clostridium] bolteae; Proteobacteria Gammaproteobacteria; Bacilli Lactobacillus; Actinomycetales Actinomycetaceae; Gammaproteobacteria Enterobacterales; Rosenbergiella collisarenosi; Rosenbergiella nectarea; Veillonellaceae Veillonella; Acidaminococcaceae Acidaminococcus; Blautia hominis; Bifidobacterium boum; Enterobacteriaceae Cedecea; Ruminococcaceae Intestinimonas; Peptostreptococcaceae Romboutsia; Fusobacteriaceae Fusobacterium; Fusobacterium nucleatum; and Megasphaera micronuciformis, is indicative of non-CDI causative diarrhea associated with IBD CD.

44) The method of claim 42, wherein a decrease in relative abundance of at least one of:
Romboutsia timonensis; Faecalibacterium prausnitzii; Coprococcus catus; Alistipes shahii; Bacteroides cellulosilyticus; Bacteroides thetaiotaomicron; Coprococcus eutactus; Turicibacter sanguinis; Colidextribacter massiliensis; and Methanobrevibacter smithii, is indicative of non-CDI causative diarrhea associated with IBD CD.

45) The method of any one of claims 42-44, wherein a change in relative abundance of at least four taxonomical features is indicative of non-CDI causative diarrhea associated with IBD CD.

46) The method of claim 21, wherein the characterization of the subject microbiome to determine whether the non-CDI causative diarrhea is associated with IBD UC or IBD CD comprises measuring one or more taxonomical features from a biological sample from the subject and determining changes in relative abundance, compared to a reference dysbiosis IBD UC gut microbiome, of at least one of:
Faecalimonas umbilicata; Blautia [Ruminococcus] gnavus; Lachnoclostridium [Clostridium] bolteae; Erysipelatoclostridium ramosum; Veillonellaceae Veillonella; Enterobacterales Enterobacteriaceae; Blautia caecimuris; Fusobacteriaceae Fusobacterium; Fusobacterium nucleatum; Faecalibacterium prausnitzii; Ruminococcaceae Oscillibacter; Ruminococcaceae Clostridium IV; Romboutsia timonensis; Ruminococcaceae Pseudoflavonifractor; Erysipelotrichales Erysipelotrichaceae; and Methanobacteriaceae Methanobrevibacter;
wherein the change in taxonomical feature relative abundance is indicative of non-CDI causative diarrhea associated with IBD CD.

47) The method of claim 46, wherein an increase in relative abundance of at least one of:
Faecalimonas umbilicata; Blautia [Ruminococcus] gnavus; Lachnoclostridium [Clostridium] bolteae; Erysipelatoclostridium ramosum; Veillonellaceae Veillonella; Enterobacterales Enterobacteriaceae; Blautia caecimuris; Fusobacteriaceae Fusobacterium; and Fusobacterium nucleatum, is indicative of non-CDI causative diarrhea associated with IBD CD.

48) The method of claim 46, wherein a decrease in relative abundance of at least one of:
Faecalibacterium prausnitzii; Ruminococcaceae Oscillibacter; Ruminococcaceae Clostridium IV; Romboutsia timonensis; Ruminococcaceae Pseudoflavonifractor; Erysipelotrichales Erysipelotrichaceae; and Methanobacteriaceae Methanobrevibacter, is indicative of non-CDI causative diarrhea associated with IBD CD.

49) The method of any one of claims 46-48, wherein a change in relative abundance of at least four taxonomical features is indicative of non-CDI causative diarrhea associated with IBD CD.

50) The method of claim 1, wherein the measuring of one or more taxonomical features from a biological sample from the individual comprises determining changes in relative abundance, compared to a reference dysbiosis gut microbiome from individuals with CDI, of at least one of:
Anaerostipes hadrus; Faecalibacterium prausnitzii; Lachnospiraceae Coprococcus; Lachnospiraceae [Eubacterium] rectale; Lachnospiraceae Roseburia; Lachnospiraceae Fusicatenibacter; Lachnospiraceae Dorea; Ruminococcaceae Ruminococcus; Roseburia inulinivorans; Dorea longicatena; Roseburia intestinalis; Lachnospiraceae Anaerostipes; Coprococcus comes; Lactobacillus rogosae; Romboutsia timonensis; Saccharibacteria Incertae Sedis; Eubacterium [Eubacterium] eligens; Bacteroides plebeius; Lachnospiraceae Blautia; Roseburia faecis; Blautia obeum; Coprococcus catus; Betaproteobacteria Burkholderiales; Prevotella copri; Burkholderiales Burkholderiaceae; Caulobacter segnis; Caulobacterales Caulobacteraceae; Burkholderiaceae Burkholderia; Burkholderia ambifaria; Prevotellaceae Prevotella; Burkholderiales Comamonadaceae; Burkholderia thailandensis; Alistipes obesi; Blautia stercoris; Ruminococcus bromii; Pelomonas aquatica; Peptostreptococcaceae Romboutsia; Bacteroides coprocola; Streptococcus thermophilus; Ruminococcaceae Oscillibacter; Alistipes ihumii; Ruminococcus callidus; Phyllobacteriaceae Phyllobacterium; Coprococcus eutactus; Peptostreptococcaceae Intestinibacter; Clostridioides difficile; Enterobacterales Enterobacteriaceae; Enterococcaceae Enterococcus; Peptostreptococcaceae Clostridium XI; Gammaproteobacteria Enterobacterales; Bacilli Lactobacillus; Eggerthella lenta; Erysipelatoclostridium [Clostridium] innocuum; Enterobacteriaceae Citrobacter; Lachnospiraceae Clostridium XIVa; Lachnoclostridium [Clostridium] bolteae; Erysipelatoclostridium ramosum; Hungatella effluvia; Veillonella parvula; Lactobacilluseae Lactobacillus; Blautia [Ruminococcus] gnavus; Enterococcus saccharolyticus; and Coriobacteriaceae Eggerthella;
wherein the change in taxonomical feature relative abundance is indicative of non-CDI causative diarrhea associated with IBS.

51) The method of claim 50, wherein an increase in relative abundance of at least one of:
Anaerostipes hadrus; Faecalibacterium prausnitzii; Lachnospiraceae Coprococcus; Lachnospiraceae [Eubacterium] rectale; Lachnospiraceae Roseburia; Lachnospiraceae Fusicatenibacter; Lachnospiraceae Dorea; Ruminococcaceae Ruminococcus; Roseburia inulinivorans; Dorea longicatena; Roseburia intestinalis; Lachnospiraceae Anaerostipes; Coprococcus comes; Lactobacillus rogosae; Romboutsia timonensis; Saccharibacteria Incertae Sedis; Eubacterium [Eubacterium] eligens; Bacteroides plebeius; Lachnospiraceae Blautia; Roseburia faecis; Blautia obeum; Coprococcus catus; Betaproteobacteria Burkholderiales; Prevotella copri; Burkholderiales Burkholderiaceae; Caulobacter segnis; Caulobacterales Caulobacteraceae; Burkholderiaceae Burkholderia; Burkholderia ambifaria; Prevotellaceae Prevotella; Burkholderiales Comamonadaceae; Burkholderia thailandensis; Alistipes obesi; Blautia stercoris; Ruminococcus bromii; Pelomonas aquatica; Peptostreptococcaceae Romboutsia; Bacteroides coprocola; Streptococcus thermophilus; Ruminococcaceae Oscillibacter; Alistipes ihumii; Ruminococcus callidus; Phyllobacteriaceae Phyllobacterium; Coprococcus eutactus; and Peptostreptococcaceae Intestinibacter, is indicative of non-CDI causative diarrhea associated with IBS.

52) The method of claim 50, wherein a decrease in relative abundance of at least one of:
Clostridioides difficile; Enterobacterales Enterobacteriaceae; Enterobacteriales Enterobacteriaceae; Enterococcaceae Enterococcus; Peptostreptococcaceae Clostridium XI; Gammaproteobacteria Enterobacterales; Bacilli Lactobacilluses; Eggerthella lenta; Erysipelatoclostridium [Clostridium] innocuum; Enterobacteriaceae Citrobacter; Lachnospiraceae Clostridium XIVa; Lachnoclostridium [Clostridium] bolteae; Erysipelatoclostridium ramosum; Hungatella effluvia; Veillonella parvula; Lactobacilluseae Lactobacillus; Blautia [Ruminococcus] gnavus; Enterococcus saccharolyticus; and Coriobacteriaceae Eggerthella, is indicative of non-CDI causative diarrhea associated with IBS.

53) The method of any one of claims 50-52, wherein a change in relative abundance of at least four taxonomical features is indicative of non-CDI causative diarrhea associated with IBS.

54) The method of claim 1, wherein the measuring of one or more taxonomical features from a biological sample from the individual comprises determining changes in relative abundance, compared to a reference dysbiosis gut microbiome from individuals with CDI, of at least one of:
Anaerostipes hadrus; Lachnospiraceae Dorea; Dorea longicatena; Lachnospiraceae Coprococcus; Lachnospiraceae Roseburia; Blautia obeum; Coprococcus comes; Roseburia intestinalis; Lactobacillus rogosae; Eubacterium [Eubacterium] eligens; Bacteroidia Bacteroidales; Peptostreptococcaceae Romboutsia; Coriobacteriaceae Collinsella; Acidaminococcaceae Acidaminococcus; Prevotellaceae Prevotella; Prevotella copri; Coriobacteriales Coriobacteriaceae; Ruminococcaceae Ruminococcus; Alistipes obesi; Betaproteobacteria Burkholderiales; Ruminococcus bromii; Lachnospiraceae Lachnoclostridium; Bifidobacterium adolescentis; Actinomycetales Actinomycetaceae; Ruminococcaceae Intestinimonas; Bacteroides eggerthii; Clostridioides difficile; Peptostreptococcaceae Clostridium XI; Enterobacteriaceae Citrobacter; Enterobacterales Enterobacteriaceae; Veillonella parvula; Enterococcaceae Enterococcus; Lactobacilluses Enterococcaceae; Erysipelatoclostridium [Clostridium] innocuum; Pectobacterium carotovorum; Enterobacteriales Enterobacteriaceae; Bacteroides xylanisolvens; Enterococcus saccharolyticus; Clostridium paraputrificum; Salmonella enterica; Erysipelatoclostridium ramosum; Hungatella effluvia; Bacteroides koreensis; Bacilli Lactobacillus; Blautia product; Klebsiella quasipneumoniae; Ruthenibacterium lactatiformans; Veillonella dispar; Coriobacteriaceae Eggerthella; and Lachnospiraceae Hungatella;
wherein the change in taxonomical feature relative abundance is indicative of non-CDI causative diarrhea associated with IBD.

55) The method of claim 54, wherein an increase in relative abundance of at least one of:
Anaerostipes hadrus; Lachnospiraceae Dorea; Dorea longicatena; Lachnospiraceae Coprococcus; Lachnospiraceae Roseburia; Blautia obeum; Coprococcus comes; Roseburia intestinalis; Lactobacillus rogosae; Eubacterium [Eubacterium] eligens; Bacteroidia Bacteroidales; Peptostreptococcaceae Romboutsia; Coriobacteriaceae Collinsella; Acidaminococcaceae Acidaminococcus; Prevotellaceae Prevotella; Prevotella copri; Coriobacteriales Coriobacteriaceae; Ruminococcaceae Ruminococcus; Alistipes obesi; Betaproteobacteria Burkholderiales; Ruminococcus bromii; Lachnospiraceae Lachnoclostridium; Bifidobacterium adolescentis; Actinomycetales Actinomycetaceae; Ruminococcaceae Intestinimonas; and Bacteroides eggerthii, is indicative of non-CDI causative diarrhea associated with IBD.

56) The method of claim 54, wherein a decrease in relative abundance of at least one of:
Clostridioides difficile; Peptostreptococcaceae Clostridium XI; Enterobacteriaceae Citrobacter; Enterobacterales Enterobacteriaceae; Veillonella parvula; Enterococcaceae Enterococcus; Lactobacillus Enterococcaceae; Erysipelatoclostridium [Clostridium] innocuum; Pectobacterium carotovorum; Enterobacteriales Enterobacteriaceae; Bacteroides xylanisolvens; Enterococcus saccharolyticus; Clostridium paraputrificum; Salmonella enterica; Erysipelatoclostridium ramosum; Hungatella effluvia; Bacteroides koreensis; Bacilli Lactobacillus; Blautia product; Klebsiella quasipneumoniae; Ruthenibacterium lactatiformans; Veillonella dispar; Coriobacteriaceae Eggerthella; and Lachnospiraceae Hungatella, is indicative of non-CDI causative diarrhea associated with IBD.

57) The method of any one of claims 54-56, wherein a change in relative abundance of at least four taxonomical features is indicative of non-CDI causative diarrhea associated with IBD.

58) A complex comprising a plurality of oligonucleotide primer sets hybridized to nucleic acid template sequences, wherein the nucleic acid template sequences are taxonomically specific sequences associated with taxonomical features identified in tables 9-17.

59) The complex of claim 58, wherein at least 5 or at least 10 oligonucleotide primer sets are hybridized to nucleic acid template sequences.

60) A kit for measuring for presence or absence or a certain level of one or more taxonomical feature(s) from a biological sample from an individual, comprising:
(a) a plurality of sets of oligonucleotide primers, wherein each set of primers hybridize to a different nucleic acid template sequence for amplifying taxonomically specific sequences; and
(b) a polymerase enzyme;
wherein the individual sets of oligonucleotide primers hybridize to a taxonomically specific sequence associated with the taxonomical features identified in tables 9-17.

61) The kit according to claim 60, wherein the master mix further comprises deoxynucleoside triphosphates; and at least one indicator for detecting an amplification product by a change in color or fluorescence.

62) The kit according to claim 61, wherein the deoxynucleoside triphosphates comprise dTTP, dGTP, dATP, dCTP and/or dUTP.

63) The kit according to claim 60, comprising at least 5, at least 10, at least 20, at least 40, at least 60, at least 80, at least 100, at least 120, at least 140, at least 160, at least 180, or at least 200 individual sets of oligonucleotide primers.

64) The kit according to claim 60, wherein the individual sets of oligonucleotide primers are bound to a support substrate.

65) The kit according to any one of claims 60 to 64, wherein the oligonucleotide primers are directed to at least 4 taxonomically specific sequences associated with the following taxonomic features: Bacteroides; Eubacterium rectale; Ruminococcus; Faecalibacterium; Enterococcus; Enterobacteriaceae; Roseburia; Coprococcus; Dorea; Lachnoclostridium; Clostridium XIVa; Erysipelatoclostridium; Alistipes; Fusicatenibacter; Odoribacter; Lactobacillus; Anaerostipes; Collinsella; Clostridioides; Klebsiella; Agathobaculum butyriciproducens; Veillonella; Phascolarctobacterium; Adlercreutzia; Clostridium; Eggerthella; Sutterellaceae Parasutterella; barnesiella; Eubacterium; Clostridium IV; Gemmiger; Streptococcus; Dialister; Escherichia; Colidextribacter; Oxalobacter; Prevotella; Clostridium XVIII; Actinomyces; and Fusobacterium.

66) The kit according to claim 65, wherein the oligonucleotide primers are directed to at least 10 taxonomically specific sequences.

67) The kit according to claim 65, wherein the oligonucleotide primers are directed to at least 20 taxonomically specific sequences.

68) The kit according to claim 65, wherein the oligonucleotide primers are directed to at least 30 taxonomically specific sequences.

69) The kit according to claim 65, wherein the oligonucleotide primers are directed to at least 40 taxonomically specific sequences.
